(19) United States
(12) Patent Application Publication
(10) Pub. No.: US 2024/0083900 A1
(43) Pub. Date: Mar. 14, 2024

(54) PYRAZOLO[1,5-A]PYRAZINE DERIVATIVES AS BTK INHIBITORS

(71) Applicant: BIOGEN MA INC., (US)

(72) Inventors: Brian T. Hopkins, Newton, MA (US); Bin Ma, Arlington, MA (US); Isaac Marx, Arlington, MA (US); Jürgen Schulz, Bonn (DE); George Vandeveer, Stoneham, MA (US); Robin Prince, Sharon, MA (US); Marta Nevalainen, Holliston, MA (US); TeYu Chen, Charlestown, MA (US); Zain Yousaf, Quincy, MA (US); Martin Himmelbauer, Cambridge, MA (US); Vatee Pattaropong, Bedford, MA (US); John Howard Jones, Framingham, MA (US); Edward Yin-Shiang Lin, Ashland, MA (US); Felix Gonzalez Lopez de Turiso, Cambridge, MA (US); Thomas Purgett, Somerville, MA (US); Andrew George Capacci, Cambridge, MA (US); Simone Sciabola, Somerville, MA (US)

(73) Assignee: BIOGEN MA INC., Cambridge, MA (US)

(21) Appl. No.: 18/036,853

(22) PCT Filed: Nov. 12, 2021
(86) PCT No.: PCT/US2021/059168

§ 371 Date: May 12, 2023

Related U.S. Application Data
(60) Provisional application No. 63/113,515, filed on Nov. 13, 2020.

(51) Int. Cl. C07D 487/04; C07D 471/04; C07D 519/00

(52) U.S. Cl. CPC C07D 487/04; C07D 471/04; C07D 519/00

(57) ABSTRACT

Provided are compounds of Formula (I′): or pharmaceutically acceptable salts thereof, wherein the variables in the formula are as defined herein; and methods for their use and production.

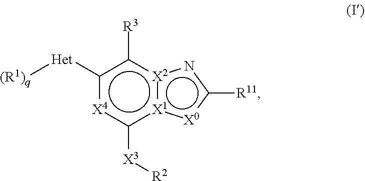

### RELATED APPLICATION

This application claims the benefit of the filing date, under 35 U.S.C. § 119(e), of U.S. Provisional Application No. 63/113,515, filed on Nov. 13, 2020, the entire contents of which are incorporated herein by reference.

### TECHNICAL FIELD

Provided are certain agents that inhibit Bruton's tyrosine kinase (Btk), and methods of making and using such agents.

### BACKGROUND

Protein kinases are a large multigene family consisting of more than 500 proteins which play a critical role in the development and treatment of a number of human diseases in oncology, neurology and immunology. The Tec kinases are non-receptor tyrosine kinases which consists of five members (Tec (tyrosine kinase expressed in hepatocellular carcinoma), Btk (Bruton's tyrosine kinase), Itk (interleukin-2 (IL-2)-inducible T-cell kinase; also known as Emt or Tsk), Rlk (resting lymphocyte kinase; also known as Txk) and Bmx (bone-marrow tyrosine kinase gene on chromosome X; also known as Etk)) and are primarily expressed in haematopoietic cells, although expression of Bmx and Tec has been detected in endothelial and liver cells. Tec kinases (Itk, Rlk and Tec) are expressed in T cell and are all activated downstream of the T-cell receptor (TCR). Btk is a downstream mediator of B cell receptor (BCR) signaling which is involved in regulating B cell activation, proliferation, and differentiation. More specifically, Btk contains a PH domain that binds phosphatidylinositol (3,4,5)-trisphosphate (PIP3). PIP3 binding induces Btk to phosphorylate phospholipase C (PLCy), which in turn hydrolyzes PIP2 to produce two secondary messengers, inositol triphosphate (IP3) and diacylglycerol (DAG), which activate protein kinase PKC, which then induces additional B-cell signaling. Mutations that disable Btk enzymatic activity result in XLA syndrome (X-linked agammaglobulinemia), a primary immunodeficiency. Given the critical roles which Tec kinases play in both B-cell and T-cell signaling, Tec kinases are targets of interest for autoimmune disorders.

Consequently, there is a great need in the art for effective inhibitors of Btk.

### SUMMARY

One embodiment of the invention is a compound represented by Formula (I′):

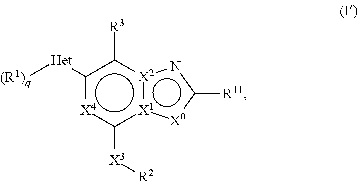

or a pharmaceutically acceptable salt thereof, wherein:
Het is phenyl, a 5-6 membered heteroaryl or a N—(C₁-C₃ alkyl)pyridonyl;
X⁰ is N, X¹ is C, X² is N and X⁴ is N; X⁰ is CR⁰, X¹ is C, X² is N and X⁴ is N; X⁰ is CR⁰, X¹ is N, X² is C and X⁴ is N; X⁰ is CR⁰, X¹ is N, X² is C and X⁴ is CH; or X⁰ is CR⁰, X¹ is C, X² is N and X⁴ is CH;
R⁰ is H, halo, methyl, halomethyl, cyclopropyl, CN or phenyl;
R¹ is H or C₁-C₃ alkyl, C₁-C₃-alkoxy, C₁-C₃ haloalkyl or a 4-7 membered monocyclic oxygen containing heterocycle;
R³ is H or halo;
X³ is absent, CH₂, CH₂CH₂, O, O—CH₂*, O—CH₂CH₂*, NH, N(CH₃)-*, CH₂N(CH₃)-* or NH—CH₂*, wherein “*” indicates the point of attachment to R²;
when X³ is absent, CH₂ or CH₂CH₂, R² is a 4-12 membered mono or bicyclic nitrogen-containing heterocycle bonded to the bicyclic core or X³ through a ring nitrogen atom (“N-attached”); when X³ is CH₂, CH₂CH₂, O, O—CH₂*, NH, N(CH₃)-*, CH₂N(CH₃)-* or NH—CH₂*, R² is a 4-12 membered mono or bicyclic nitrogen containing heterocycle bonded to X³ through a ring carbon atom (“C-attached”), a 4-7 membered mono or bicyclic oxygen containing heterocycle, a 3-12 membered mono or bicyclic carbocyclyl, or a 5-6 membered heteroaryl; and when X³ is O—CH₂—CH₂*, R² is absent, a 4-12 membered mono or bicyclic nitrogen containing heterocycle bonded to X³ through a ring carbon atom (“C-attached”) or a C₁-C₃ alkyl group, provided when R² is absent, X³ is directly connected to R⁴;
the N-attached 4-12 membered mono or bicyclic nitrogen-containing heterocycle, the 4-7 membered oxygen containing heterocycle, the 3-12 membered mono or bicyclic carbocycle, the 5-6 membered heteroaryl, and the C₁-C₃ alkyl group represented by R² are substituted with a group represented by R⁴ and optionally further substituted with one to three groups represented by R¹⁰, provided when the N-attached 4-12 membered mono or bicyclic nitrogen-containing heterocycle contains two ring nitrogen atoms, the N-attached 4-12 membered mono or bicyclic nitrogen-containing heterocycle represented by R² is optionally N-substituted with a group represented by R⁵ and optionally further substituted with one or two groups represented by R¹⁰;
the C-attached 4-12 membered mono or bicyclic nitrogen-containing heterocycle is N-substituted with a group represented by R⁵ and optionally further substituted with one to three groups represented by R¹⁰;
R⁴ is

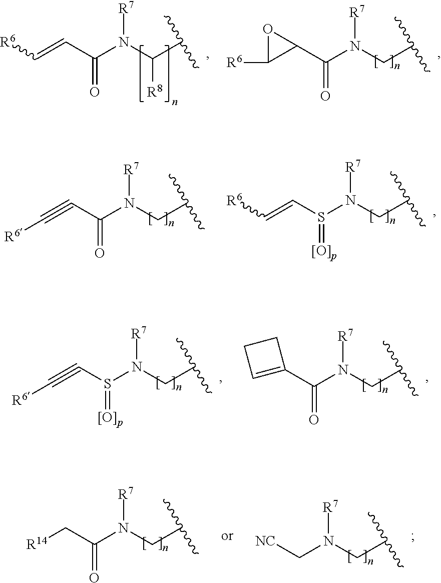

R⁵ is

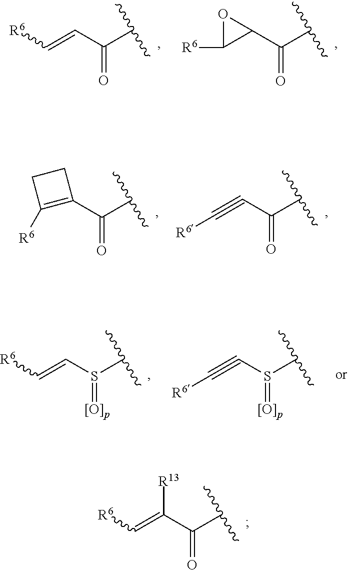

each R⁶ is independently H, CN, C₁-C₃ alkyl, C₁-C₃ haloalkyl, N(R^{a})₂ or CH₂N(R^{a})₂, wherein each R^{a} is independently H, C₁-C₃ alkyl or C₃-C₆ cycloalkyl;
each R⁶′ is independently H, C₁-C₃ alkyl, C₁-C₃ haloalkyl or C₃-C₆ cycloalkyl;
each R⁷ is independently H, C₁-C₂ alkyl, C₁-C₂ fluoroalkyl or C₃-C₆ cycloalkyl;
R⁸ is H or C₁-C₃ alkyl;
each R¹⁰ is halo, C₁-C₃ alkyl or C₃-C₆ cycloalkyl;
R¹¹ is H or N(R¹²)₂;
each R¹² is independently H or C₁-C₃ alkyl;
R¹³ is CN or F;
R¹⁴ is halo;
each n is independently 0 or 1;
each p is independently 1 or 2; and
q is 1 or 2.

Another embodiment of the invention is a compound of Formula (I):

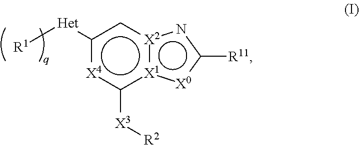

or a pharmaceutically acceptable salt thereof, wherein:

Het is phenyl, a 5-6 membered heteroaryl or an N—(C₁-C₃ alkyl) pyridonyl;
X⁰ is N, X¹ is C, X² is N and X⁴ is N; X⁰ is CR⁰, X¹ is C, X² is N and X⁴ is N; X⁰ is CR⁰, X¹ is N, X² is C and X⁴ is N; X⁰ is CR⁰, X¹ is N, X² is C and X⁴ is CH; or X⁰ is CR⁰, X¹ is C, X² is N and X⁴ is CH;
R⁰ is H, halo, methyl, halomethyl, cyclpropyl or CN;
R¹ is H or C₁-C₃ alkyl, C₁C₃-alkoxy, C₁-C₃ haloalkyl or a 4-7 membered monocyclic oxygen containing heterocycle;
X³ is absent, CH₂, CH₂CH₂, O, O—CH₂*, NH or NH—CH₂*, wherein “*” indicates the point of attachment to R²;
when X³ is absent, CH₂ or CH₂CH₂, R² is a 4-12 membered mono or bicyclic nitrogen-containing heterocycle bonded to the bicyclic core or X³ through a ring nitrogen atom (“N-attached”); and when X³ is CH₂, CH₂CH₂, O, O—CH₂* or NH—CH₂*, R² is a 4-12 membered mono or bicyclic nitrogen containing heterocycle bonded to X³ through a ring carbon atom (“C-attached”), a 4-7 membered monocyclic oxygen containing heterocycle or a 3-12 membered mono or bicyclic carbocyclyl;
the N-attached 4-12 membered mono or bicyclic nitrogen-containing heterocycle, the 4-7 membered oxygen containing heterocycle and the 3-12 membered mono or bicyclic carbocycle represented by R² are substituted with a group represented by R⁴ and optionally further substituted with one or two groups represented by R¹⁰;
the C-attached 4-12 membered mono or bicyclic nitrogen-containing heterocycle is N-substituted with a group represented by R⁵ and optionally further substituted with one or two groups represented by R¹⁰;
R⁴ is

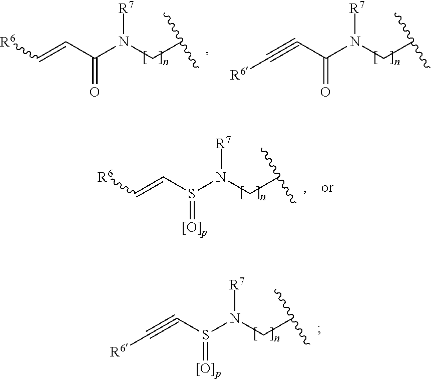

R⁵ is

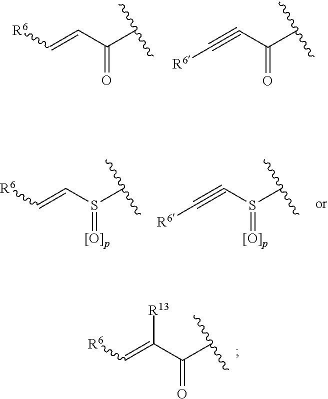

each R⁶ is independently H, C₁-C₃ alkyl, C₁-C₃ haloalkyl, N(R^{a})₂ or CH₂N(R^{a})₂, wherein each R^{a} is independently H or methyl;
each R⁶′ is independently H, C₁-C₃ alkyl or C₁-C₃ haloalkyl;
each R⁷ is independently H, C₁-C₂ alkyl or C₁-C₂ fluoroalkyl;
each R¹⁰ is F or methyl;
R¹¹ is H or N(R¹²)₂. Alternatively, R¹¹ is H or NH₂;
each R¹² is independently H or C₁-C₃ alkyl; alternatively, R¹² is H or NH₂;
R¹³ is CN or F;
each n is independently 0 or 1;
each p is independently 1 or 2; and
q is 1 or 2.

The present invention also provides a pharmaceutical composition comprising at least one compound described herein, or a pharmaceutically acceptable salt thereof, and at least one pharmaceutically acceptable excipient.

Another embodiment of the invention is a method of treating a disorder responsive to inhibition of Btk in a subject comprising administering to the subject an effective amount of at least one compound described herein, or a pharmaceutically acceptable salt thereof.

The present invention also includes the use of at least one compound described herein, or a pharmaceutically acceptable salt thereof, for the manufacture of a medicament for the treatment of a disorder responsive to inhibition of Btk. Also provided is a compound described herein, or a pharmaceutically acceptable salt thereof for use in treating a disorder responsive to inhibition of Btk.

Other features or advantages will be apparent from the following detailed description of several embodiments, and also from the appended claims.

### DETAILED DESCRIPTION

The compounds or pharmaceutically acceptable salts thereof, as described herein, can have activity as Btk modulators. In particular, compounds or pharmaceutically acceptable salts thereof, as described herein, can be Btk inhibitors.

In a first embodiment, the compound of the invention is represented by Formula (I′) or a pharmaceutically acceptable salt thereof, wherein the variables are as described above.

In a second embodiment, the compound of the invention is represented by Formula (I) or a pharmaceutically acceptable salt thereof, wherein the variables are as described above.

In a third embodiment, for the compound of Formula (I′) or (I), or a pharmaceutically acceptable salt thereof, R¹¹ is H or NH₂, and the remainder of the variables are as described in the first or second embodiment.

In a fourth embodiment, the compound of the invention is represented by Formula (II):

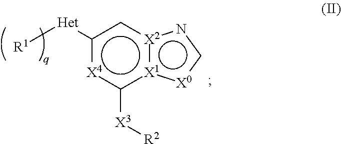

or a pharmaceutically acceptable salt thereof. The variables in Formula (II) are as described for Formula (I′) or (I) described in the first or second embodiment.

In a fifth embodiment, the compound of the invention is represented by Formula (I′), (I) or (II), or a pharmaceutically acceptable salt thereof, wherein (R¹)_{q}-Het- in Formulas (I′), (I) and (II) is selected from:

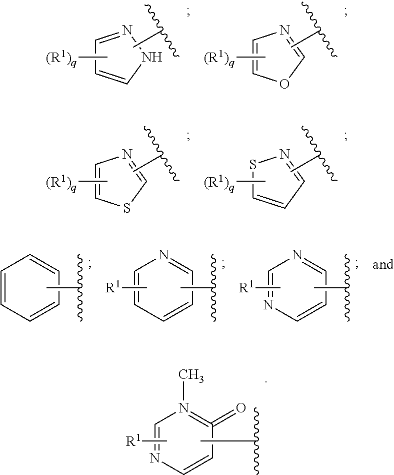

The remainder of the variables in Formulas (I), (I′) and (II) are as described in any one of the first through fourth embodiments.

In a sixth embodiment, the compound of the invention is represented by Formula (III):

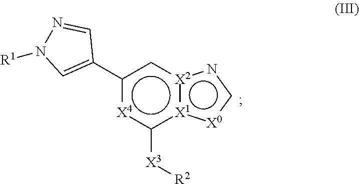

or a pharmaceutically acceptable salt thereof. The variables in Formula (III) are as described for Formula (I′) or (I) described in the first or second embodiment.

In a seventh embodiment, the compound of the invention is represented by Formula (I′), (I), (II) or (III), or a pharmaceutically acceptable salt thereof, wherein X⁰ is N, X¹ is C, X² is N and X⁴ is N; X⁰ is CH, X¹ is C, X² is N and X⁴ is N; X⁰ is CH, X¹ is N, X² is C and X⁴ is N; X⁰ is CR⁰, X¹ is N, X² is C and X⁴ is CH; or X⁰ is CH, X¹ is C, X² is N and X⁴ is CH; X³ is absent, O, O—CH₂*, NH or NH—CH₂*, wherein “*” indicates the point of attachment to R²; when X³ is absent, R² is a 4-12 membered monocyclic or bicyclic nitrogen-containing heterocycle bonded to the bicyclic core or X³ through a ring nitrogen atom (“N-attached”); and when X³ is O, O—CH₂* or NH—CH₂*, R² is a 4-12 membered monocyclic or bicyclic nitrogen containing heterocycle bonded to X³ through a ring carbon atom (“C-attached”), a 4-7 membered monocyclic oxygen containing heterocycle or a 3-12 membered monocyclic or bicyclic carbocyclyl; the N-attached 4-12 membered monocyclic or bicyclic nitrogen-containing heterocycle, the 4-7 membered monocyclic oxygen-containing and the 3-12 membered monocyclic or bicyclic carbocycle represented by R² are substituted with a group represented by R⁴ and optionally further substituted with one or two groups represented by R¹⁰; the C-attached 4-12 membered monocyclic or bicyclic nitrogen-containing heterocycle is N-substituted with a group represented by R⁵ and optionally further substituted with one or two groups represented by R¹⁰; and the remainder of the variables are as described for Formulas (I′), (I), (II) and (III) as described in any one of the first through sixth embodiments.

In an eighth embodiment, the compound of the invention is represented by Formula (IV), (V), (VI), (VII) or (VIII):

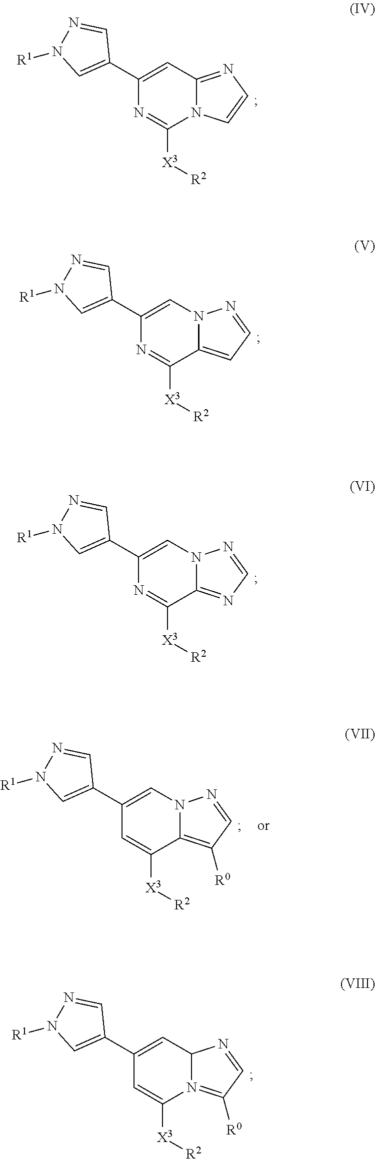

or a pharmaceutically acceptable salt of any of the foregoing, wherein the variables are as described in any one of the first through seventh embodiments.

In a ninth embodiment, the compound of the invention is represented by any one of Formulas (I′), (I), (II), (III), (IV), (V), (VI), (VII) and (VIII), or a pharmaceutically acceptable salt thereof, wherein X³ is a bond and R² is a 4-12 membered monocyclic or bicyclic nitrogen-containing heterocycle bonded to the bicyclic core through a ring nitrogen atom and the monocyclic or bicyclic 4-12 membered nitrogen-containing heterocycle represented by R² is substituted with a group represented by R⁴ and optionally further substituted with a group represented by R¹⁰. Alternatively, R² is a 4-7 membered monocyclic nitrogen-containing heterocycle bonded to the bicyclic core through a ring nitrogen atom and the 4-7 membered monocyclic nitrogen-containing heterocycle represented by R² is substituted with a group represented by R⁴ and optionally further substituted with a group represented by R¹⁰. The remainder of the variables are as described for Formula (I′), (I), (II), (III), (IV), (V), (VI), (VII) or (VIII) in any one of the first through eight embodiments.

In a tenth embodiment, the compound of the invention is represented by any one of Formulas (I′), (I), (II), (III), (IV), (V), (VI), (VII) and (VIII), or a pharmaceutically acceptable salt thereof, wherein X³ is a bond and R² is a 7-10 membered bicyclic nitrogen-containing heterocycle bonded to the bicyclic core through its ring nitrogen atom and the 7-10 membered bicyclic nitrogen-containing heterocycle represented by R² is substituted with a group represented by R⁴ and optionally further substituted with one or two groups represented by R¹⁰; and the remainder of the variables are as described in any one of the first through ninth embodiments.

In an eleventh embodiment, the compound of the invention is represented by any one of Formulas (I′), (I), (II), (III), (IV), (V), (VI), (VII) and (VIII), or a pharmaceutically acceptable salt thereof, wherein the 7-10 membered bicyclic nitrogen-containing heterocycle represented by R² is azaspiro[2.4]heptanylene substituted with a group represented by R⁴ and optionally further substituted with a group represented by R¹⁰; and the remainder of the variables are as described in any one of the first through tenth embodiments.

In a twelfth embodiment, the compound of the invention is represented by any one of (I′), (I), (II), (III), (IV), (V), (VI), (VII) and (VIII), or a pharmaceutically acceptable salt thereof, wherein X³ is a bond and R² is a 4-7 membered monocyclic nitrogen-containing heterocycle bonded to the bicyclic core through its ring nitrogen atom and the 4-7 membered monocyclic nitrogen-containing heterocycle represented by R² is substituted with a group represented by R⁴ and optionally further substituted with a group represented by R¹⁰; and the remainder of the variables are as described in any one of the first through ninth embodiments.

In a thirteenth embodiment, the compound of the invention is represented by any one of (I′), (I), (II), (III), (IV), (V), (VI), (VII) and (VIII), or a pharmaceutically acceptable salt thereof, wherein the 4-7 membered monocyclic or bicyclic nitrogen-containing heterocycle represented by R² is azetidinylene, pyrrolindinylene, piperidinylene, azapanylene or oxazapanylene, each substituted with a group represented by R⁴ and optionally further substituted with a group represented by R¹⁰, and the remainder of the variables are as described in any one of the first through ninth embodiments.

In a fourteenth embodiment, the compound of the invention is represented by Formula (IX), (X), (XI), (XII), (XIII) or (XIV):

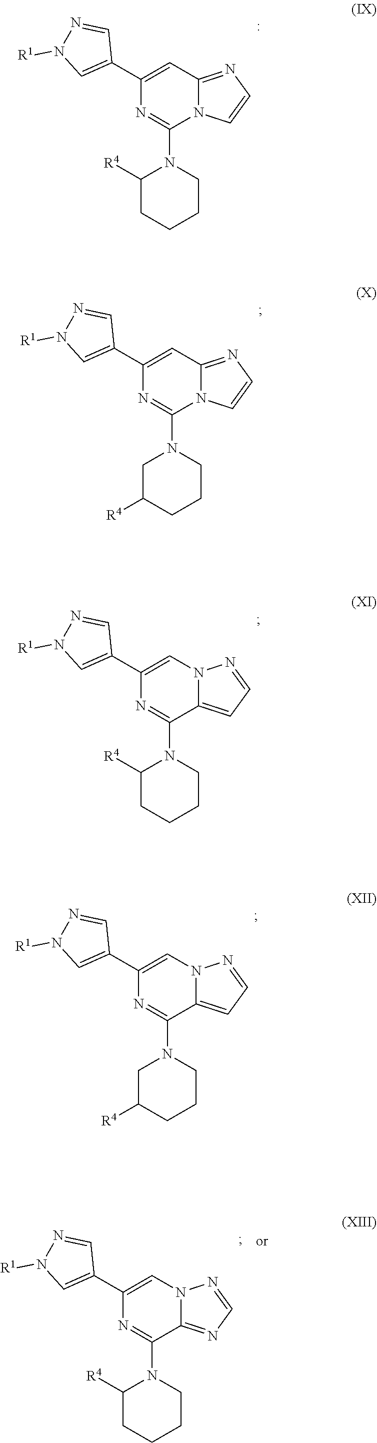

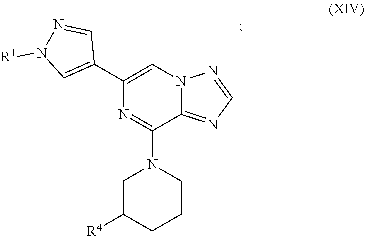

or a pharmaceutically acceptable salt of any of the foregoing, wherein the variables are as described in the thirteenth embodiment.

In a fifteenth embodiment, the compound of the invention is represented by any one of Formulas (I′), (I), (II), (III), (IV), (V), (VI), (VII), (VIII), (IX), (X), (XI), (XII), (XIII) or (XIV), wherein R⁶ and R⁶′ are independently H, CH₃ or CH₂Cl, p is 2, and the remainder of the variables are as described in any one of the first through fourteenth embodiments.

In an sixteenth embodiment, the compound of the invention is represented by any one of Formulas (I′), (I), (II), (III), (IV), (V), (VI), (VII), (VIII), (IX), (X), (XI), (XII), (XIII) or (XIV), wherein R⁴ is CH₂NHC(O)C≡CH, CH₂NHC(O)CH═CH₂, N(CH₃)C(O)C≡CH, NHC(O)CH═CH₂, NHC(O)C≡CH or NHC(O)CH═CHCH₂Cl; and the remainder of the variables are as described in any one the first through fifteenth embodiments.

In a seventeenth embodiment, the compound of the invention is represented by any one of Formulas (I′), (I), (II), (III), (IV), (V), (VI), (VII), (VIII), (IX), (X), (XI), (XII), (XIII) or (XIV), wherein R⁴ is CH₂NHC(O)C≡CH, CH₂NHC(O)CH═CH₂, N(CH₃)C(O)C≡CH or CH₂NR⁷C(O)CH═CHCH₂Cl, and the remainder of the variables are as described in any one of the first through fifteenth embodiments.

In an eighteenth embodiment, the compound of the invention is represented by any one of Formulas (I′), (I), (II), (III), (IV), (V), (VI), (VII) or (VIII), wherein X³ is O, O—CH₂*, O—CH₂CH₂*, NH, NH—CH₂*, N(CH₃), or CH₂N(CH₃)-*, R² is a 4-12 membered mono or bicyclic nitrogen containing heterocycle bonded to X³ through a ring carbon atom (“C-attached”) and the C-attached 4-12 membered nitrogen-containing heterocycle is N-substituted with a group represented by R⁵ and optionally further substituted with one to three groups represented by R¹⁰; and the remainder of the variables are as described in any one of the first through eighth embodiments.

In a ninteenth embodiment, the compound of the invention is represented by any one of Formulas (I′), (I), (II), (III), (IV), (V), (VI), (VII) or (VIII), wherein X³ is O, O—CH₂*, NH or NH—CH₂*, R² is a 4-12 membered nitrogen containing heterocycle bonded to X³ through a ring carbon atom (“C-attached”) and the C-attached 4-12 membered nitrogen-containing heterocycle is N-substituted with a group represented by R⁵ and optionally further substituted with a group represented by R¹⁰; and the remainder of the variables are as described in any one of the first through eighth embodiments.

In a twentieth embodiment, the compound of the invention is represented by any one of Formulas (I′), (I), (II), (III), (IV), (V), (VI), (VII) or (VIII), X³ is O or O—CH₂*, and the remainder of the variables are as described in any one of the first through eighth, eighteenth and ninteenth embodiments.

In a twenty-first embodiment, the compound of the invention is represented by any one of Formulas (I′), (I), (II), (III), (IV), (V), (VI), (VII) or (VIII), wherein the C-attached 4-12 membered nitrogen containing heterocycle represented by R² is a 4-7 membered monocyclic heterocycle optionally containing one ring oxygen or one ring sulfur atom, 6-10 membered fused bucyclic, an 8-12 membered spirocycle or 7-10 bridged bicyclic and the C-attached 4-12 membered nitrogen containing heterocycle represented by R² is N-substituted with a group represented by R⁵ and optionally further substituted with a group represented by R¹⁰; and the remainder of the variables are as described in the first through eighth and eighteenth through twentieth embodiments.

In a twenty-second embodiment, the compound of the invention is represented by any one of Formulas (I′), (I), (II), (III), (IV), (V), (VI), (VII) or (VIII), wherein the C-attached 4-12 membered nitrogen containing heterocycle represented by R² is azaspiro[3.3]heptanylene, azaspiro[3.5]nonanylene, azaspiro[4.4]nonanylene, azaspiro[3.4]octanylene, azetidinylene, pyrrolindinylene, piperidinylene, azapanylene, diazepanylene, morpholinylene, octahydrocyclopenta[c]pyrrolylene, oxazapanylene, azabicyclo[3.2.0]heptanylene, azabicyclo[2.2.1]heptanylene, azabicyclo[3.1.1]heptanylene, azabicyclo[3.2.1]octanylene, azabicyclo[4.2.0]octanylene, azatricyclo[4.1.1.03,7]octylene, azabicyclo[3.2.0]heptanylene, azabicyclo[2.1.1]heptanylene, azabicyclo[2.1.1]hexanylene, azabicyclo[3.1.0]hexanylene, 2λ²-azaspiro[3.4]octylene or octahydrocyclopenta[c]pyrolene and the C-attached 4-12 membered nitrogen containing heterocycle represented by R² is N-substituted with a group represented by R⁵ and is optionally further substituted with one or two groups represented by R¹⁰; and the remainder of the variables are as described in any one of the first through eighth and eighteenth through twenty-first embodiments. Exemplary 4-12 membered nitrogen containing heterocycles represented by R² include

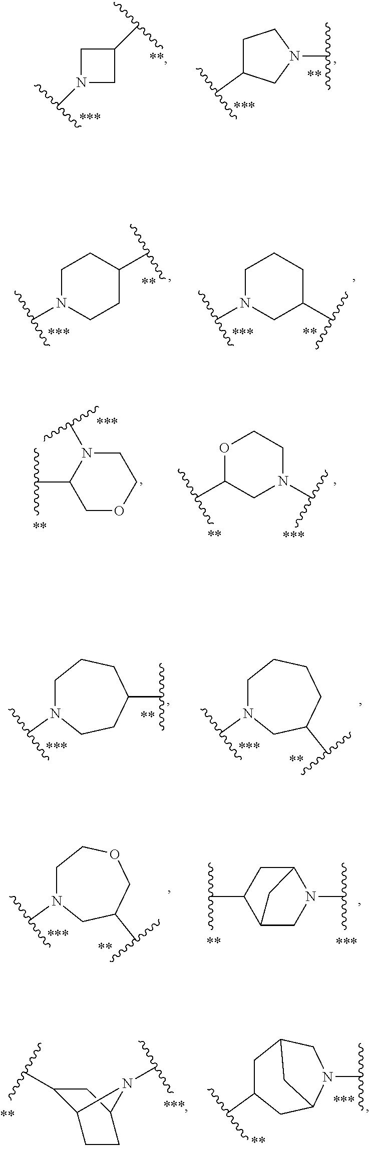

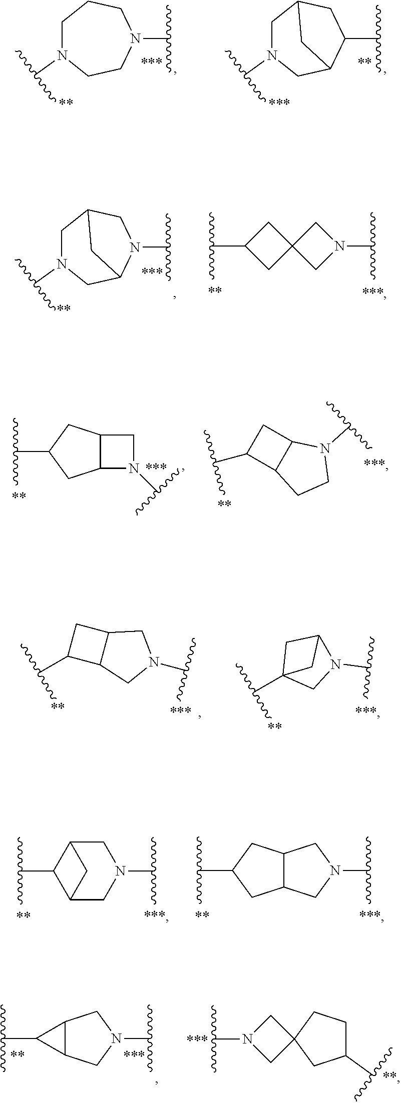

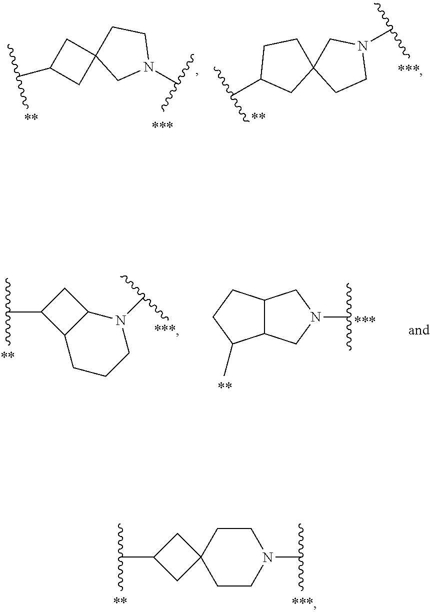

wherein “**” indicates the point of attachment to X³; and “***” indicates the point of attachment to R⁵, wherein each group represented by R² is optionally further substituted with one to three groups represented by R¹⁰.

In a twenty-third embodiment, the compound of the invention is represented by any one of Formulas (I′), (I), (II), (III), (IV), (V), (VI), (VII) or (VIII), wherein the C-attached 4-12 membered nitrogen containing heterocycle represented by R² is azetidinylene, pyrrolindinylene, piperidinylene, azapanylene, oxazapanylene, azabicyclo[3.2.1]octanylene, azatricyclo[4.1.1.0^{3,7}]octylene, azabicyclo[3.2.0]heptanylene, azabicyclo[3.1.0]hexanylene, 2λ²-azaspiro[3.4]octylene or octahydrocyclopenta[c]pyrollene and the C-attached 4-12 membered nitrogen containing heterocycle represented by R² is N-substituted with a group represented by R⁵ and is optionally further substituted with one or two groups represented by R¹⁰ and the remainder of the variables are as described in any one of the first through eighth and eighteenth through twenty-first embodiments. Exemplary 4-12 membered nitrogen containing heterocycles represented by R² include

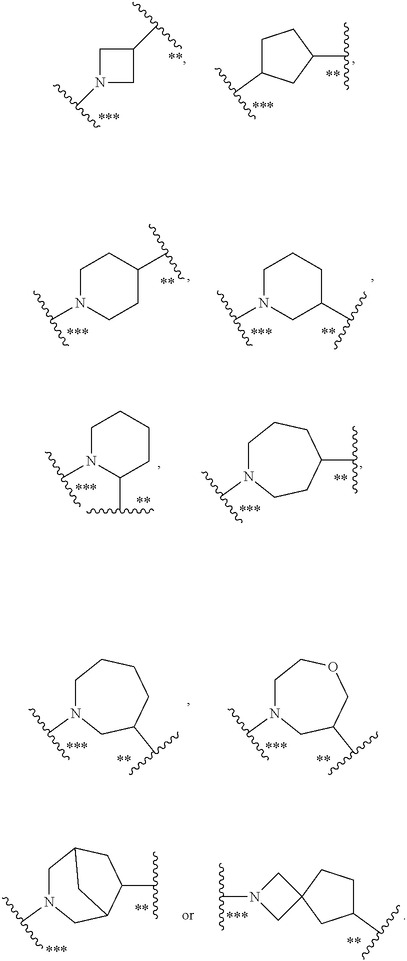

The nitrogen containing heterocycle represented by R² is optionally further substituted with R¹⁰; “**” indicates the point of attachment to X³; and “***” indicates the point of attachment to R⁵, wherein each group represented by R² is optionally further substituted with one or two groups represented by R¹⁰.

In a twenty-fourth embodiment, the compound of the invention is represented by any one of Formulas (I′), (I), (II), (III), (IV), (V), (VI), (VII) or (VIII), wherein the stereochemical configuration of the ring carbon atom in the C-attached 4-12 membered nitrogen containing heterocycle represented by R² that is bonded to X³ is R. Alternatively, the stereochemical configuration of the ring carbon atom in the C-attached 4-12 membered nitrogen containing heterocycle represented by R² that is bonded to X³ is S. The remainder of the variables in Formulas (I′), (I), (II), (III), (IV), (V), (VI), (VII) or (VIII) are as described in any one of the first through eighth and eighteenth through twenty-third embodiments.

In a twenty-fifth embodiment, the compound of the invention is represented by any one of Formulas (I′), (I), (II), (III), (IV), (V), (VI), (VII) or (VIII), wherein R⁶ and R⁶′ are independently H, CH₃ or CH₂Cl and p is 2; and the remainder of the variables are as described in any one of the first through eighth and eighteenth through twenty-fourth embodiments.

In a twenty-sixth embodiment, the compound of the invention is represented by any one of Formulas (I′), (I), (II), (III), (IV), (V), (VI), (VII) or (VIII), wherein R⁵ is SO₂CH═CH₂, SO₂CH═CHCH₃, SO₂CH═CHCH₂Cl, SO₂C≡CH, SO₂C≡CCH₃, SO₂C≡CCH₂Cl, COCH═CH₂, COCH═CHCH₃, COCH═CHCH₂Cl, CO—C≡CH, CO—C≡CCH₃, CO—C≡CCH₂Cl, COCF═CH₂, COCF═CHCH₃, COCF═CHCH₂Cl,

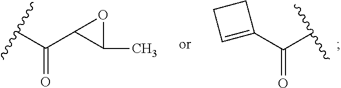

and the remainder of the variables are as described in the first through eighth and eighteenth through twenty-fifth embodiments.

In a twenty-seventh embodiment, the compound of the invention is represented by any one of Formulas (I′), (I), (II), (III), (IV), (V), (VI), (VII) or (VIII), wherein R⁵ is SO₂CH═CH₂, SO₂CH═CHCH₃, SO₂CH═CHCH₂Cl, SO₂C≡CH, SO₂C≡CCH₃, SO₂C≡CCH₂Cl, COCH═CH₂, COCH═CHCH₃, COCH═CHCH₂Cl, CO—C≡CH, CO—C≡CCH₃ or CO—C≡CCH₂Cl. Alternatively, R⁵ is SO₂CH═CH₂, SO₂CH═CHCH₃, COCH═CH₂, COCH═CHCH₂Cl, CO—C≡CH or CO—C≡CCH₃. The remainder of the variables in Formulas (I′), (I), (II), (III), (IV), (V), (VI), (VII) or (VIII) are as described in any one the first through eighth and eighteenth through twenty-fifth embodiments.

In a twenty-eighth embodiment, the compound of the invention is represented by any one of Formulas (I′), (I), (II), (III), (IV), (V), (VI), (VII) or (VIII), wherein X³ is O, O—CH₂*, NH or NH—CH₂*, R² is a 3-12 membered mono or bicyclic carbocyclyl, a 4-7 membered mono or bicyclic oxygen containing heterocycle or a 5-6 membered heteroaryl and the 3-12 membered mono or bicyclic carbocycle, the 4-7 membered mono or bicyclic oxygen containing heterocycle and the 5-6 membered heteroaryl represented by R² is substituted with a group represented by R⁴ and optionally further substituted with one to three groups represented by R¹⁰; and the remainder of the variables are as described in any one of the first through eighth embodiments.

In a twenty-ninth embodiment, the compound of the invention is represented by any one of Formulas (I′), (I), (II), (III), (IV), (V), (VI), (VII) or (VIII), wherein X³ is O, O—CH₂*, NH or NH—CH₂*, R² a 4-7 membered mono or bicyclic oxygen containing heterocycle or a 5-6 membered heteroaryl and the 4-7 membered mono or bicyclic oxygen containing heterocycle and the 5-6 membered heteroaryl represented by R² are substituted with a group represented by R⁴ and optionally further substituted with one to three groups represented by R¹⁰; and the remainder of the variables are as described in any one of the first through eighth embodiments.

In a thirtieth embodiment, the compound of the invention is represented by any one of Formulas (I′), (I), (II), (III), (IV), (V), (VI), (VII) or (VIII), wherein the 4-7 membered mono or bicyclic oxygen containing heterocycle represented by R² is oxabicyclo [3.1.1]heptanylene or tetrahydro-2H-pyranylene, each substituted with a group represented by R⁴ and optionally further substituted with one or two groups represented by R¹⁰; and the 5-6 membered heteroaryl is pyridinylene substituted with a group represented by R⁴ and optionally further substituted with one to three groups represented by R¹⁰; and the remainder of the variables are as described in any one of the first through eighth and twenty-ninth embodiments.

In a thirty-first embodiment, the compound of the invention is represented by any one of Formulas (I′), (I), (II), (III), (IV), (V), (VI), (VII) or (VIII), wherein R² is selected from:

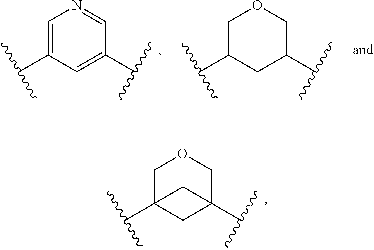 [SMILES: *c1cncc(*)c1 ; *C1COCC(*)C1 ; *C12COCC(*)(C1)C2]

each substituted with a group represented by R⁴ and optionally further substituted with one or two groups represented by R¹⁰; and the remainder of the variables are as described in any one of the first through eighth and twenty-ninth embodiments.

In a thirty-second embodiment, the compound of the invention is represented by any one of Formulas (I′), (I), (II), (III), (IV), (V), (VI), (VII) or (VIII), wherein X³ is O, O—CH₂*, NH or NH—CH₂*, R² is a 3-12 membered mono or bicyclic carbocyclyl and the 3-12 membered mono or bicyclic carbocycle represented by R² is substituted with a group represented by R⁴ and optionally further substituted with one or two groups represented by R¹⁰, and the remainder of the variables are as described in any one of the first through eighth embodiment. Alternatively, X³ is O or O—CH₂*. In another alternative, X³ is O. The remainder of the variables in Formulas (I′), (I), (II), (III), (IV), (V), (VI), (VII) or (VIII) are as described in any one of the first through eighth embodiments.

In a thirty-third embodiment, the compound of the invention is represented by any one of Formulas (I′), (I), (II), (III), (IV), (V), (VI), (VII) or (VIII), wherein R² is phenylene, C₃-C₇ cycloalkylene or C₆-C₉ bicyclic saturated carbocycle and the phenylene, C₃-C₇ cycloalkylene and C₆-C₉ bicyclic saturated carbocycle represented by R² is substituted with a group represented by R⁴ and optionally further substituted with one or two groups represented by R¹⁰; and the remainder of the variables in are as described in any one of the first through eighth and twenty-eighth through thirty-second embodiments.

In a thirty-fourth embodiment, the compound of the invention is represented by any one of Formulas (I′), (I), (II), (III), (IV), (V), (VI), (VII) or (VIII), wherein R² is phenylene or C₄-C₇ cycloalkylene substituted with a group represented by R⁴ and optionally further substituted with one or two groups represented by R¹⁰; and the remainder of the variables are as described in any one of the first through eighth, thirty-second and thirty-third embodiments.

In a thirty-fifth embodiment, the compound of the invention is represented by any one of Formulas (I′), (I), (II), (III), (IV), (V), (VI), (VII) or (VIII), wherein X³ is O; and the remainder of the variables are as described in any one of the first through eighth and twenty-eighth through thirty-fourth embodiments.

In a thirty-sixth embodiment, the compound of the invention is represented by any one of Formulas (I′), (I), (II), (III), (IV), (V), (VI), (VII) or (VIII), wherein R² is phenylene, cyclobutylene, cyclohexylene, cyclopentylene, cyclopropylene, bicyclo[3.3.1]heptylene, bicyclo[2.2.1]heptanylene, bicyclo[4.1.0]heptanylene or bicyclo[2.1.1]hexanylene, each of which is substituted with a group represented by R⁴ and optionally further substituted with one or two groups represented by R¹⁰; and the remainder of the variables are as described in any one of the first through eighth and thirty-second through thirty-fifth embodiments.

In a thirty-seventh embodiment, the compound of the invention is represented by any one of Formulas (I′), (I), (II), (III), (IV), (V), (VI), (VII) or (VIII), wherein R² is phenylene, cyclobutylene, cyclohexylene or bicycle [3.3.1]heptylene substituted with a group represented by R⁴ and optionally further substituted with one or two groups represented by R¹⁰; and the remainder of the variables are as described in the first through eighth and thirty-second through thirty-fifth embodiments.

In a thirty-eighth embodiment, the compound of the invention is represented by any one of Formulas (I′), (I), (II), (III), (IV), (V), (VI), (VII) or (VIII), wherein R² is

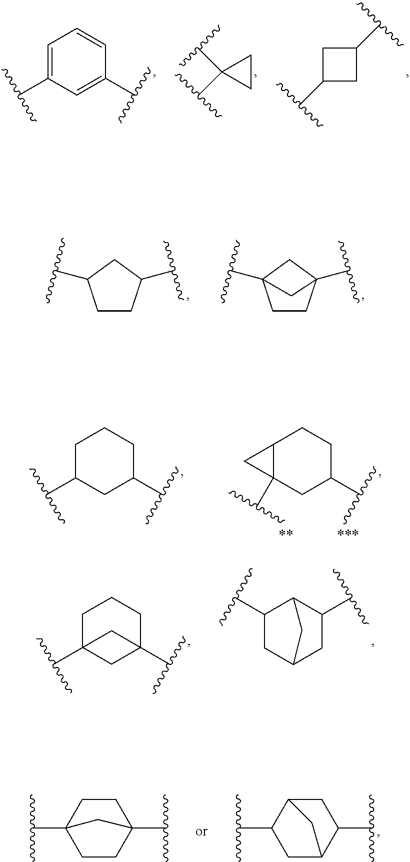

wherein “**” indicates the point of attachment to X³; and “***” indicates the point of attachment to R⁴, wherein the group represented by R² is optionally substituted with one or two groups represented by R¹⁰. The remainder of the variables in Formulas (I′), (I), (II), (III), (IV), (V), (VI), (VII) or (VIII) are as described in the first through eighth and thirty-second through thirty-fifth embodiments.

In a thirty-ninth embodiment, the compound of the invention is represented by any one of Formulas (I′), (I), (II), (III), (IV), (V), (VI), (VII) or (VIII), wherein R² is

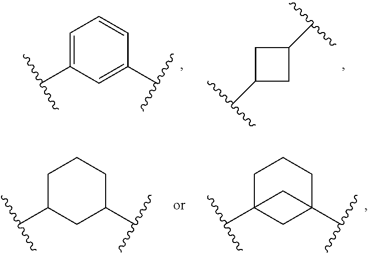 [SMILES: *c1cccc(*)c1 ; *C1CC(*)C1 ; *C1CCCC(*)C1 ; *C12CCCC(*)(C1)C2]

wherein the group represented by R² is optionally substituted with one or two groups represented by R¹⁰; and the remainder of the variables are as described in any one of the first through eighth or thirty-second through thirty-fifth embodiments.

In a fortieth embodiment, the compound of the invention is represented by any one of Formulas (I′), (I), (II), (III), (IV), (V), (VI), (VII) or (VIII), wherein R⁶ and R⁶′ are independently H, CN, CH₃, CH₂Cl, CF₃, cyclopropyl or CH₂N(R^{a}); and the remainder of the variables are as described in any one of the first through eighth and thirty-second through thirty-ninth embodiments.

In a forty-first embodiment, the compound of the invention is represented by any one of Formulas (I′), (I), (II), (III), (IV), (V), (VI), (VII) or (VIII), wherein R^{a} are each independently selected from —CH₃ and cyclopropyl; and the remainder of the variables are as described in any one of the first through eighth and thirty-second through fortieth embodiments.

In a forty-second embodiment, the compound of the invention is represented by any one of Formulas (I′), (I), (II), (III), (IV), (V), (VI), (VII) or (VIII), wherein R⁶ and R^{6′} are each independently H, CH₃ or CH₂Cl; and the remainder of the variables are as described in any one of the first through eighth and thirty-second through thirty-ninth embodiments.

In a forty-third embodiment, the compound of the invention is represented by any one of Formulas (I′), (I), (II), (III), (IV), (V), (VI), (VII) or (VIII), wherein R⁴ is NHC(O)CH═CH₂, N(CH₃)C(O)CH═CH₂, NHC(O)CH═CHCH₃, N(CH₃)C(O)CH═CHCH₃, N(CH₃)C(O)CH═CHCN, NHC(O)C≡CH, N(CH₃)C(O)C≡CH, N(H)C(O)C≡CCH₃, N(CH₃)C(O)C≡CCH₃, N(CH₂CH₂F)C(O)CH═CH₂, N(CH₂CH₂F)C(O)CH═CHCH₃, N(CH₂CH₂F)C(O)C≡CH, N(CH₂CH₂F)C(O)C≡CCH₃, CH₂N(CH₃)C(O)CH═CH₂, N(CH₂CHF₂)C(O)CH═CH₂, N(CH₃)C(O)CH═CHCH₂Cl, NHC(O)CH═CHCF₃, N(CH₃)C(O)CH═CHCF₃, NHC(O)C≡C-cyclopropyl, NHC(O)CH═CHCH₂N(CH₃)-cyclobutyl, N(CH₂CHF₂)C(O)CH═CHCH₂N(CH₃)₂, N(cyclopropyl)C(O)CH═CH₂, N(CH₃)C(O)CH₂Cl, N(CH₃)CH₂CN,

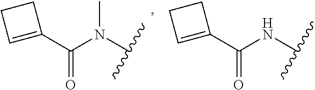

CH₂NHC(O)CH═CH₂, or CH(CH₃)NHC(O)CH═CH₂. The remainder of the variables in Formulas (I′), (I), (II), (III), (IV), (V), (VI), (VII) or (VIII) are as described in any one of the first through eighth and thirty-second through forty-first embodiments.

In a forty-fourth embodiment, the compound of the invention is represented by any one of Formulas (I′), (I), (II), (III), (IV), (V), (VI), (VII) or (VIII), R⁴ is NHCOCH═CH₂, N(CH₃)COCH═CH₂, NHCOCH═CHCH₃, N(CH₃)COCH═CHCH₃, N(H)COC≡CH, N(CH₃)COC≡CH, N(H)COC≡CCH₃, N(CH₃)COC≡CCH₃, N(CH₂CH₂F)COCH═CH₂, N(CH₂CH₂F)COCH═CHCH₃, N(CH₂CH₂F)COC≡CH or N(CH₂CH₂F)COC≡CCH₃. Alternatively, R⁴ is NHC(O)C≡CH, NHC(O)C≡CCH₃, NHC(O)CH═CH₂, N(CH₃)COCH═CH₂, N(CH₃)COC≡CCH₃ or N(CH₂CH₂F)COCH═CH₂. The remainder of the variables in Formulas (I′), (I), (II), (III), (IV), (V), (VI), (VII) or (VIII) are as described in any one of the first through eighth and thirty-second through forty-second embodiments.

In a forty-fifth embodiment, the compound of the invention is represented by any one of Formulas (I′), (I), (II), (III), (IV), (V), (VI), (VII) or (VIII), wherein the stereochemical configuration of the ring carbon atom in the C-attached 3-12 membered carbocycle represented by R² that is bonded to X³ is R. Alternatively, the stereochemical configuration of the ring carbon atom in the C-attached 3-12 membered carbocycle represented by R² that is bonded to X³ is S. The remainder of the variables in Formulas (I′), (I), (II), (III), (IV), (V), (VI), (VII) or (VIII) are as described in any one of the first through eighth and thirty-second through forty-fourth embodiments.

In a forty-sixth embodiment, the compound of the invention is represented by any one of Formulas (I′), (I), (II), (III), (IV), (V), (VI), (VII) or (VIII), wherein X³ and R⁴ are orientated trans. Alternatively, X³ and R⁴ are orientated cis. The remainder of the variables in Formulas (I′), (I), (II), (III), (IV), (V), (VI), (VII) or (VIII) are as described in any one of the first through eighth and thirty-second through forty-fourth embodiments.

In a forty-seventh embodiment, the compound of the invention is represented by any one of Formulas (I′), (I), (II), (III), (IV), (V), (VI), (VII) or (VIII), wherein X³ is O—CH₂CH₂*, and R² is C₁-C₃ alkyl group substituted with a group represented by R⁴ and optionally further substituted with one or two groups represented by R¹, or R² is absent and X³ is directly connected to R⁴. The remainder of the variables in Formulas (I′), (I), (II), (III), (IV), (V), (VI), (VII) or (VIII) are as described in the first through eighth embodiments.

In a forty-eighth embodiment, the compound of the invention is represented by any one of Formulas (I′), (I), (II), (III), (IV), (V), (VI), (VII) or (VIII), wherein R² is selected from **-CH₂-***, **-CH₂CH(CH₃)-***, wherein “**” represents a point of attachment to X³, and “***” represents a point of attachment to R⁴. The remainder of the variables in Formulas (I′), (I), (II), (III), (IV), (V), (VI), (VII) or (VIII) are as described in any one of the first through eighth and forty-seventh embodiments.

In a forty-ninth embodiment, the compound of the invention is represented by any one of Formulas (I′), (I), (II), (III), (IV), (V), (VI), (VII) or (VIII), wherein R⁴ is N(CH₃)C(O)CH═CH₂; and the remainder of the variables are as described in any one of the first through eighth, forty-seventh and forty-eighth embodiments.

In a fiftieth embodiment, the compound of the invention is represented by any one of Formulas (I′), (I), (II), (III), (IV), (V), (VI), (VII), (VIII), (IX), (X), (XI), (XII), (XIII) or (XIV), wherein R¹ is H or C₁-C₃ alkyl, C₁-C₃ fluoroalkyl or a 4-7 membered monocyclic oxygen containing heterocycle. Alternatively, R¹ is H, CH₃, CH(CH₃)₂, CHF₂, oxetanyl or tetrahydrofuranyl. The remainder of the variables in Formulas (I′), (I), (II), (III), (IV), (V), (VI), (VII), (VIII), (IX), (X), (XI), (XII), (XIII) or (XIV) are as described in any one of the first through forty-ninth embodiments.

In a fifty-first embodiment, the compound of the invention is represented by any one of Formulas (I′), (I), (II), (III), (IV), (V), (VI), (VII), (VIII), (IX), (X), (XI), (XII), (XIII) or (XIV), wherein R¹ is H, CH₃, CH(CH₃)₂, CHF₂, CF₃, oxetanyl or tetrahydrofuranyl; and the remainder of the variables are as described in any one of the first through forty-ninth embodiments.

In a fifty-second embodiment, the compound of the invention is represented by any one of Formulas (I′), (I), (II), (III), (IV), (V), (VI), (VII), (VIII), (IX), (X), (XI), (XII), (XIII) or (XIV), wherein R⁰ is H, F, CN, CH₃, CF₃, cyclopropyl or phenyl; and the remainder of the variables are as described in any one of the first through fifty-first embodiments.

In a fifty-third embodiment, the compound of the invention is represented by any one of Formulas (I′), (I), (II), (III), (IV), (V), (VI), (VII), (VIII), (IX), (X), (XI), (XII), (XIII) or (XIV), wherein R⁰ is H, F, CN, CH₃ or CF₃; and the remainder of the variables are as described in any one of the first through fifty-first embodiments.

In a fifty-fourth embodiment, the compound of the invention is represented by any one of Formulas (I′), (I), (II), (III), (IV), (V), (VI), (VII), (VIII), (IX), (X), (XI), (XII), (XIII) or (XIV), wherein R⁷ is selected from H, CH₃, CH₂CH₃, CH₂CHF₂ and cyclopropyl; and the remainder of the variables are as described in any one of the first through fifty-third embodiments.

In a fifty-fifth embodiment, the compound of the invention is represented by any one of Formulas (I′), (I), (II), (III), (IV), (V), (VI), (VII), (VIII), (IX), (X), (XI), (XII), (XIII) or (XIV), wherein R¹ is H or CH₃; and the remainder of the variables are as described in any one of the first through fifty-fourth embodiments.

In a fifty-sixth embodiment, the compound of the invention is represented by any one of Formulas (I′), (I), (II), (III), (IV), (V), (VI), (VII), (VIII), (IX), (X), (XI), (XII), (XIII) or (XIV), wherein R¹⁰ is F, Cl, CH₃ or cyclopropyl; and the remainder of the variables in Formulas (I′), (I), (II), (III), (IV), (V), (VI), (VII), (VIII), (IX), (X), (XI), (XII), (XIII) or (XIV) are as described in any one of the first through fifty-fifth embodiments.

In a fifty-seventh embodiment, the compound of the invention is represented by any one of Formulas (I′), (I), (II), (III), (IV), (V), (VI), (VII), (VIII), (IX), (X), (XI), (XII), (XIII) or (XIV), wherein R¹⁴ is Cl; and the remainder of the variables are as described in any one of the first through fifty-sixth embodiments.

In a fifty-eighth embodiment, the compound is represented by the following formula:

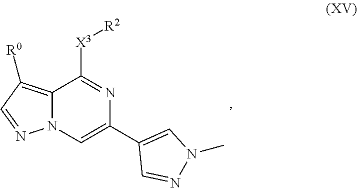

or a pharmaceutically acceptable salt thereof, wherein: R¹ is H, halo or cyclopropyl; X³ is O or O—CH₂*; R² is a 4-7 membered monocyclic or bicyclic saturated carbocyclyl and the 4-7 membered monocyclic or bicyclic saturated carbocyclyl represented by R² is substituted with a group represented by R⁴ and optionally further substituted with one or two R¹⁰, or R² is a 7-9 membered bicyclic nitrogen containing heterocycle bonded to X³ through a ring carbon atom (“C-attached”) and the C-attached 7-9 membered bicyclic nitrogen containing heterocycle is substituted with a group represented by R⁵ and optionally further substituted with one or two R¹⁰; R⁴ is N(R⁷)C(O)C≡CCH₃, N(R⁷)C(O)CH═CH₂, R¹ is C(O)CH═CH₂, R⁷ is H, C₁-C₂alkyl, or C₁-C₂haloalkyl; and R¹⁰ is C₁-C₃alkyl.

In a fifty-ninth embodiment, the compound of the invention is represented by Formula (XV), wherein X³ is O; and the remainder of the variables in Formula (XV) are as described in the fifty-eighth embodiment.

In a sixtieth embodiment, the compound of the invention is represented by Formula (XV), wherein R² is cyclobutylene, cyclohexylene, cyclopentylene or bicyclo [2.1.1]hexanylene, each of which is substituted with a group represented by R⁴ and optionally further substituted with one or two R¹⁰. The remainder of the variables in Formula (XV) are as described in the fifty-eighth or fifty-ninth embodiment.

In a sixty-first embodiment, the compound of the invention is represented by Formula (XV), wherein R², or

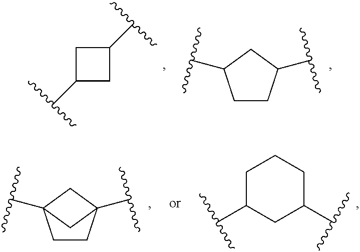 [SMILES: *C1CC(*)C1 ; *C1CCC(*)C1 ; *C12CCC(*)(C1)C2 ; *C1CCCC(*)C1]

wherein the group represented by R² is optionally further substituted with one or two groups represented by R¹⁰. The remainder of the variables in Formula (XV) are as described in the fifty-eighth or fifty-ninth embodiment.

In a sixty-second embodiment, the compound of the invention is represented by Formula (XV), wherein R² is azabicyclo[3.2.1]octanylene, azabicyclo[3.1.1]heptanylene or azabicyclo[3.2.0]heptanylene, each of which is substituted with a group represented by R⁵ and optionally further substituted with one or two R¹⁰. The remainder of the variables in Formula (XV) are as described in the fifty-eighth or fifty-ninth embodiment.

In a sixty-third embodiment, the compound of the invention is represented by Formula (XV), wherein R² is

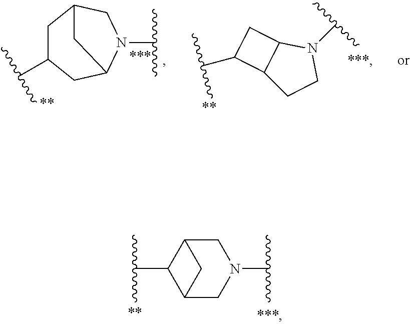 [SMILES: *C1CC2CC(C1)N(*)C2 ; *C1CC2C1CCN2* ; *C1C2CC1CN(*)C2]

wherein “**” indicates the point of attachment to X³; and “***” indicates the point of attachment to R⁵, wherein each group represented by R² is optionally further substituted with one or two groups represented by R¹⁰. The remainder of the variables in Formula (XV) are as described in any one of the fifty-eighth through sixty-second embodiments.

In a sixty-fourth embodiment, the compound of the invention is represented by Formula (XV), wherein R⁷ is H, CH₃ or CH₂CHF₂. The remainder of the variables in Formula (XV) are as described in an one of the fifty-eighth through sixty-third embodiments.

In a sixty-fifth embodiment, the compound of the invention is represented by Formula (XV), wherein R¹⁰ is CH₃. The remainder of the variables in Formula (XV) are as described in any one of the fifty-eighth through sixty-fourth embodiments.

The invention also includes both the neutral form and pharmaceutically acceptable salts of the compounds disclosed in the exemplification.

As used herein, the term “alkyl” refers to a fully saturated branched or unbranched hydrocarbon moiety. Unless otherwise specified, an alkyl comprises 1 to 6 carbon atoms, or 1 to 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, or n-hexyl.

As used herein, the term “alkoxy” refers to a fully saturated branched or unbranched alkyl moiety attached through an oxygen bridge (i.e. a —O—C₁₋₄ alkyl group, wherein C₁₋₄ alkyl is as defined herein). Representative examples of alkoxy include, but are not limited to, methoxy, ethoxy, propoxy, 2-propoxy, butoxy, tert-butoxy and the like. In some embodiments, alkoxy groups have about 1-4 carbons, more preferably about 1-2 carbons.

The number of carbon atoms in a group is specified herein by the prefix “Cₓ₋ₓₓ”, wherein x and xx are integers. For example, “C₁₋₃ alkyl” is an alkyl group which has from 1 to 3 carbon atoms.

“Halogen” or “halo” may be fluoro, chloro, bromo or iodo.

The term “haloalkyl” or “halo-substituted alkyl” refers to an alkyl group having at least one halogen substitution. The term “fluoroalkyl” or “fluoro-substituted alkyl” refers to an alkyl group having at least one fluorine substitution.

“Heterocyclyl” or “heterocycle” refers to a saturated or partially unsaturated monocyclic or bicyclic (e.g., fused, bridged or spiro ring systems) ring system which has from 4- to 12-ring members, at least one of which is a heteroatom, and up to 4 (e.g., 1, 2, 3, or 4) of which may be heteroatoms, wherein the heteroatoms are independently selected from O, S and N, and wherein C can be oxidized (e.g., C(O)), N can be oxidized (e.g., N(O)) or quaternized, and S can be optionally oxidized to sulfoxide and sulfone. In some embodiments, if a “heterocyclyl” or “heterocycle” described herein contains both N and O, then the “heterocyclyl” or “heterocycle” is considered to be a N-containing heterocycle.

A 4-12 membered heterocyclyl can be a monocyclic 4 to 7 membered heterocyclyl or a bicyclic 7 to 12 membered heterocyclcyl that is fused, bridged or spiro. Examples of 4- to 7-membered monocyclic heterocyclyl include, but are not limited to, oxetanyl, thietanyl, azetedinyl, pyrrolidinyl, tetrahydrofuranyl, thiolanyl, imidazolidinyl, pyrazolidinyl, oxazolidinyl, isoxazolidinyl, thiazolidinyl, isothiazolidinyl, dioxolanyl, dithiolanyl, oxathiolanyl, piperidinyl, tetrahydropyranyl, thianyl, piperazinyl, morpholinyl, thiomorpholinyl, dioxanyl, dithianyl, trioxanyl, trithianyl, azepanyl, oxepanyl, thiepanyl, dihydrofuranyl, imidazolinyl, and dihydropyranyl.

A “fused ring system” has from 8 to 12 members (ring atoms) and two rings which share two adjacent ring atoms. A fused bicyclic heterocyclyl has a 4 to 7 membered heterocycyl fused to a 4 to 7 membered heterocycyl or a 3 to 7 membered non-aromatic carbocyclyl. Examples include cyclopentapyrrolidinyl, cyclopentapiperidinyl, cyclopentaazapanyl, cyclohexapyrrolidinyl, cyclohexapiperidinyl, cyclohexaazapanyl, cycloheptapyrrolidinyl, cycloheptapiperidinyl, cycheptaazapanyl, pyrrolopyrrolidinyl, pyrrolopiperidinyl, pyrroloazapanyl, furanopyrrolidinyl, furanopiperidinyl, furanoazapanyl, pyranopyrrolidinyl, pyranopiperidinyl, pyranoazapanyl and the like.

A “bridged bycyclic ring system” (also referred to herein as a “bridged bicyclic”) has 7 to 10 members (ring atoms) and two rings which share three adjacent ring atoms. A bridged bicyclic heterocyclyl comprises a 5 to 7 membered heterocycyl which shares three ring atoms with a 5 to 7 membered heterocycyl or a 5 to 7 membered non-aromatic carbocyclyl. Examples nitrogen containing bridged bicyclics include azabicyclo[2.2.1]hepantyl, azabicyclo[3.2.1]octanyl, azabicyclo [3.3.1]nonanyl, diazabicyclo[2.2.1]hepantyl, diazabicyclo[3.2.1]octanyl and diazabicyclo [3.3.1]nonanyl. Examples of oxygen containing bridged bicyclics include oxobicyclo [2.2.1]hepantyl, oxobicyclo [3.2.1]octanyl, oxobicyclo [3.3.1]nonanyl, oxa-azabicyclo [2.2.1]hepantyl, oxa-azabicyclo [3.2.1]octanyl and oxa-azabicyclo [3.3.1]nonanyl.

A “spiro ring system” (also referred to herein as a “spirocycle”) has 8 to 12 members (ring atoms) and two rings which share one ring atom. A spirobicyclic heterocyclyl comprises a 4 to 7 membered heterocycyl which shares one atom with a 4 to 7 membered heterocycyl or a 4 to 7 membered non-aromatic carbocyclyl. Examples of 8 to 12 nitrogen containing spiro rings systems include 3,4-azabicyclooctanyl, 4,4-azabicyclononanyl, 3,5-azabicyclononanyl, 3,6-azabicyclodecanyl, 4,5-azabicyclodecanyl, 3,7-azabicycloundecanyl, 4,6-azabicycloundecanyl and 5,5-azabicycloundecanyl. Examples of 8-12 oxygen containing spiro ring systems include 3,4-oxobicyclooctanyl, 4,4-oxobicyclononanyl, 3,5-oxobicyclononanyl, 3,6-oxobicyclodecanyl, 4,5-oxobicyclodecanyl, 3,7-oxobicycloundecanyl, 4,6-oxobicycloundecanyl and 5,5-xobicycloundecanyl.

Examples of 4 to 12 membered nitrogen containing heterocycles include pyrrolidinyl, imidazolidinyl, pyrazolidinyl, oxazolidinyl, isoxazolidinyl, thiazolidinyl, isothiazolidinyl, piperidinyl, piperazinyl, morpholinyl, thiomorpholinyl, azepanyl, oxepanyl, imidazolinyl, cyclopentapyrrolidinyl, cyclopentapiperidinyl, cyclopentaazapanyl, cyclohexapyrrolidinyl, cyclohexapyrrolidinyl, cyclohexaazapanyl, cycloheptapyrrolidinyl, cycloheptapyrrolidinyl, cycloheptaazapanyl, pyrrolopyrolidinyl, pyrrolopiperidinyl, pyrroloazapanyl, furanopiperidinyl, furanoazapanyl, pyranopyrrolidinyl, pyranopiperidinyl, pyranoazapanyl, azabicyclo[2.2.1]hepantyl, azabicyclo[3.2.1]octanyl, azabicyclo [3.3.1]nonanyl, diazabicyclo[2.2.1]hepantyl, diazabicyclo[3.2.1]octanyl, diazabicyclo [3.3.1]nonanyl, 3,4-azabicyclooctanyl, 4,4-azabicyclononanyl, 3,5-azabicyclononanyl, 3,6-azabicyclodecanyl, 4,5-azabicyclodecanyl, 3,7-azabicycloundecanyl, 4,6-azabicycloundecanyl and 5,5-azabicycloundecanyl. Examples of 4 to 7 membered nitrogen containing heterocycles (optionally containing one ring oxygen or one ring sulfur atom) include pyrrolidinyl, imidazolidinyl, pyrazolidinyl, oxazolidinyl, isoxazolidinyl, thiazolidinyl, isothiazolidinyl, piperidinyl, piperazinyl, morpholinyl, thiomorpholinyl, azepanyl, oxepanyl and imidazolinyl.

Examples of 4 to 7 membered oxygen containing heterocycles include oxetanyl, tetrahydrofuranyl, oxazolidinyl, isoxazolidinyl, dioxolanyl, oxathiolanyl, tetrahydropyranyl, morpholinyl, dioxanyl, oxepanyl, dihydrofuranyl and dihydropyranyl.

“Heteroaryl” refers to an aromatic 5- to 6-membered monocyclic ring system, having 1 to 4 heteroatoms independently selected from O, N and S, and wherein N can be oxidized (e.g., N(O)) or quaternized, and S can be optionally oxidized to sulfoxide and sulfone. Examples of 5- to 6-membered monocyclic heteroaryls include, but are not limited to, pyrrolyl, furanyl, thiophenyl (or thienyl), imidazolyl, pyrazolyl, oxazolyl, isoxazolyl, thiazolyl, isothiazolyl, furazanyl, oxadiazolyl, thiadiazolyl, dithiazolyl, triazolyl, tetrazolyl, pyridinyl, pyranyl, thiopyranyl, pyrazinyl, pyrimidinyl, pyridazinyl, oxazinyl, thiazinyl, dioxinyl, dithiinyl, oxathianyl, triazinyl, tetrazinyl, and the like. In one embodiment, a heteroaryl is a 5-membered heteroaryl. Examples of a 5-membered heteroaryl include, but are not limited to, pyrazolyl, oxazolyl, isoxazolyl, 1,2,3-oxadiazolyl, 1,3,4-oxadiazolyl, 1,2,4-oxadizolyl, 1,2,3-thiadiazolyl, 1,3,4-thiadiazolyl, 1,2,4-thiadiazolyl, 1,2,3-triazolyl, 1,2,4-triazolyl, and tetrazolyl.

“Carbocyclyl” refers to a saturated or partially unsaturated monocyclic or bicyclic (e.g., fused, bridged or spiro ring systems) ring system which has from 4- to 12-ring members, all of which are carbon. The term “carbocyclyl” encompasses cycloalkyl groups, cycloalkenyl group and aromatic groups (i.e., aryl). “Cycloalkyl” refers to completely saturated monocyclic hydrocarbon groups of 3-7 carbon atoms, including cyclopropyl, cyclobutyl, cyclpentyl, cyclohexyl and cyclopentyl; and “cycloalkyenyl” refers to unsaturated non-aromatic monocyclic hydrocarbon groups of 3-7 carbon atoms, including cyclpentenyl, cyclohexenyl and cyclopentenyl. Exemplary aromatic carbocyclyl groups include phenyl.

A fused bicyclic carbocyclyl has a 4 to 7 membered carbocycyl fused to a 3 to 7 membered non-aromatic carbocyclyl. Examples include decahydronapthalene, octahydro-1H-indene, octahydropentalene, decahydroazulene, decahydro-1H-annulene, bicycle [4.2.0]octane, bicyclel[3.2.0]heptane and the like.

A bridged bicyclic carbocyclyl comprises a non-aromatic 5 to 7 membered carbocyclyl which shares three ring atoms with a 5 to 7 membered non-aromatic carbocyclyl. Examples od bridged bicyclics carbocycles include bicyclo[2.2.1]hepantyl, bicyclo[3.2.1]octanyl, bicyclo [3.3.1]nonanyl,

The suffic “yl” added to the end of a chemical name indicates that the named moiety is bonded to the molecule at point. The suffix “ene” added to the end of a chemical name indicates that the named moiety is bonded to the molecule at two points. Examples include azetidinylene, pyrrolindinylene, piperidinylene, azapanylene or oxazapanylene, which indicates that an azetidine, pyrrolidine, piperidine, azapane or oxazapane is bonded to the remainder of the compound at two points.

In the context of a nitrogen-containing heterocycle, “N-attached to the bicyclic core” means that the nitrogen-containing heterocycle is bonded to the core

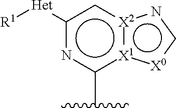

though its ring nitrogen atom. In the context of a nitrogen-containing heterocycle or a carbocycle, “C-attached to the bicyclic core” means that the nitrogen-containing heterocycle or carbocycle is bonded to the core

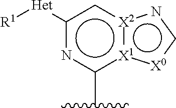

through a ring carbon atom.

A nitrogen-containing heterocycle is “N-substituted” when a ring nitrogen atom is substituted.

In cases where a compound provided herein is sufficiently basic or acidic to form stable nontoxic acid or base salts, preparation and administration of the compounds as pharmaceutically acceptable salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, or α-glycerophosphate. Inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Pharmaceutically-acceptable base addition salts can be prepared from inorganic and organic bases. Salts from inorganic bases, can include but are not limited to, sodium, potassium, lithium, ammonium, calcium or magnesium salts. Salts derived from organic bases can include, but are not limited to, salts of primary, secondary or tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocycloalkyl amines, diheterocycloalkyl amines, triheterocycloalkyl amines, or mixed di- and tri-amines where at least two of the substituents on the amine can be different and can be alkyl, substituted alkyl, alkenyl, substituted alkenyl, cycloalkyl, substituted cycloalkyl, cycloalkenyl, substituted cycloalkenyl, aryl, heteroaryl, or heterocycloalkyl and the like. Also included are amines where the two or three substituents, together with the amino nitrogen, form a heterocycloalkyl or heteroaryl group. Non-limiting examples of amines can include, isopropylamine, trimethyl amine, diethyl amine, tri(iso-propyl) amine, tri(n-propyl) amine, ethanolamine, 2-dimethylaminoethanol, trimethamine, lysine, arginine, histidine, caffeine, procaine, hydrabamine, choline, betaine, ethylenediamine, glucosamine, N-alkylglucamines, theobromine, purines, piperazine, piperidine, morpholine, or N-ethylpiperidine, and the like. Other carboxylic acid derivatives can be useful, for example, carboxylic acid amides, including carboxamides, lower alkyl carboxamides, or dialkyl carboxamides, and the like.

The compounds or pharmaceutically acceptable salts thereof as described herein, can contain one or more asymmetric centers in the molecule. In accordance with the present disclosure any structure that does not designate the stereochemistry is to be understood as embracing all the various stereoisomers (e.g., diastereomers and enantiomers) in pure or substantially pure form, as well as mixtures thereof (such as a racemic mixture, or an enantiomerically enriched mixture). It is well known in the art how to prepare such optically active forms (for example, resolution of the racemic form by recrystallization techniques, synthesis from optically-active starting materials, by chiral synthesis, or chromatographic separation using a chiral stationary phase).

When a particular stereoisomer of a compound is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired stereoisomer relative to the combined weight of all stereoisomers.

When a particular enantiomer of a compound is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired enantiomer relative to the combined weight of all stereoisomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers are included. It is to be further understood that the stereoisomeric purity of the named or depicted stereoisomers is at least 20%, 30%, 40%, 50%, 60%, 70%, 80%, 90%, 95%, 97%, 99%, 99.5% or 99.9%. The stereoisomeric purity the weight percent of the desired stereoisomers encompassed by the name or structure relative to the combined weight of all of the stereoisomers.

When a disclosed compound is named or depicted by structure without indicating the stereochemistry, and the compound has one chiral center, it is to be understood that the name or structure encompasses one enantiomer of compound in pure or substantially pure form, as well as mixtures thereof (such as a racemic mixture of the compound and mixtures enriched in one enantiomer relative to its corresponding optical isomer).

When a disclosed compound is named or depicted by structure without indicating the stereochemistry and, e.g., the compound has at least two chiral centers, it is to be understood that the name or structure encompasses one stereoisomer in pure or substantially pure form, as well as mixtures thereof (such as mixtures of stereoisomers, and mixtures of stereoisomers in which one or more stereoisomers is enriched relative to the other stereoisomer(s)).

The disclosed compounds may exist in tautomeric forms and mixtures and separate individual tautomers are contemplated. In addition, some compounds may exhibit polymorphism.

In one embodiment, the invention provides deuterated compounds disclosed herein, in which any or more positions occupied by hydrogen can include enrichment by deuterium above the natural abundance of deuterium. For example, one or more hydrogen atoms are replaced with deuterium at an abundance that is at least 3340 times greater than the natural abundance of deuterium, which is 0.015% (i.e., at least 50.1% incorporation of deuterium), at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). In one embodiment, hydrogen is present at all positions at its natural abundance. The compounds or pharmaceutically acceptable salts thereof as described herein, may exist in tautomeric forms and mixtures and separate individual tautomers are contemplated.

Another embodiment is a pharmaceutical composition comprising at least one compound described herein, or a pharmaceutically acceptable salt thereof, and at least one pharmaceutically acceptable carrier.

The compounds, or pharmaceutically acceptable salts thereof described herein may be used to decrease the activity of Btk, or to otherwise affect the properties and/or behavior of Btk, e.g., stability, phosphorylation, kinase activity, interactions with other proteins, etc.

In some embodiments, the present invention provides methods of decreasing Btk enzymatic activity. In some embodiments, such methods include contacting a Btk with an effective amount of a Btk inhibitor. Therefore, the present invention further provides methods of inhibiting Btk enzymatic activity by contacting a Btk with a Btk inhibitor of the present invention.

One embodiment of the invention includes a method of treating a disorder responsive to inhibition of Btk in a subject comprising administering to the subject an effective amount of at least one compound described herein, or a pharmaceutically acceptable salt thereof.

In one embodiment, the present invention provides methods of treating autoimmune disorders, inflammatory disorders, and cancers in a subject in need thereof comprising administering to the subject an effective amount of at least one compound described herein, or a pharmaceutically acceptable salt thereof.

The term “autoimmune disorders” includes diseases or disorders involving inappropriate immune response against native antigens, such as acute disseminated encephalomyelitis (ADEM), Addison's disease, alopecia areata, antiphospholipid antibody syndrome (APS), autoimmune hemolytic anemia, autoimmune hepatitis, bullous pemphigoid (BP), Coeliac disease, dermatomyositis, diabetes mellitus type 1, Goodpasture's syndrome, Graves' disease, Guillain-Barre syndrome (GBS), Hashimoto's disease, idiopathic thrombocytopenic purpura, lupus erythematosus, mixed connective tissue disease, multiple sclerosis, myasthenia gravis, pemphigus vulgaris, pernicious anaemia, polymyositis, primary biliary cirrhosis, Sjogren's syndrome, temporal arteritis, and Wegener's granulomatosis. The term “inflammatory disorders” includes diseases or disorders involving acute or chronic inflammation such as allergies, asthma, prostatitis, glomerulonephritis, pelvic inflammatory disease (PID), inflammatory bowel disease (IBD, e.g., Crohn's disease, ulcerative colitis), reperfusion injury, rheumatoid arthritis, transplant rejection, and vasculitis. In some embodiments, the present invention provides a method of treating rheumatoid arthritis or lupus. In some embodiments, the present invention provides a method of treating multiple sclerosis.

The term “cancer” includes diseases or disorders involving abnormal cell growth and/or proliferation, such as glioma, thyroid carcinoma, breast carcinoma, lung cancer (e.g. small-cell lung carcinoma, non-small-cell lung carcinoma), gastric carcinoma, gastrointestinal stromal tumors, pancreatic carcinoma, bile duct carcinoma, ovarian carcinoma, endometrial carcinoma, prostate carcinoma, renal cell carcinoma, lymphoma (e.g., anaplastic large-cell lymphoma), leukemia (e.g. acute myeloid leukemia, T-cell leukemia, chronic lymphocytic leukemia), multiple myeloma, malignant mesothelioma, malignant melanoma, and colon cancer (e.g. microsatellite instability-high colorectal cancer). In some embodiments, the present invention provides a method of treating leukemia or lymphoma.

As used herein, the term “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the term “treating” or ‘treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder; or delaying, inhibiting or decreasing the likelihood of the progression of the disease, disorder or syndrome.

The effective dose of a compound provided herein, or a pharmaceutically acceptable salt thereof, administered to a subject can be 10 μg-500 mg.

Administering a compound described herein, or a pharmaceutically acceptable salt thereof, to a mammal comprises any suitable delivery method. Administering a compound described herein, or a pharmaceutically acceptable salt thereof, to a mammal includes administering a compound described herein, or a pharmaceutically acceptable salt thereof, topically, enterally, parenterally, transdermally, transmucosally, via inhalation, intracisternally, epidurally, intravaginally, intravenously, intramuscularly, subcutaneously, intradermally or intravitreally to the mammal. Administering a compound described herein, or a pharmaceutically acceptable salt thereof, to a mammal also includes administering topically, enterally, parenterally, transdermally, transmucosally, via inhalation, intracisternally, epidurally, intravaginally, intravenously, intramuscularly, subcutaneously, intradermally or intravitreally to a mammal a compound that metabolizes within or on a surface of the body of the mammal to a compound described herein, or a pharmaceutically acceptable salt thereof.

Thus, a compound or pharmaceutically acceptable salt thereof as described herein, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compound or pharmaceutically acceptable salt thereof as described herein may be combined with one or more excipients and used in the form of ingestible tablets, buccal tablets, troches, capsules, elixirs, suspensions, syrups, or wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions can be such that an effective dosage level will be obtained.

The tablets, troches, pills, capsules, and the like can include the following: binders such as gum tragacanth, acacia, corn starch or gelatin; excipients such as dicalcium phosphate; a disintegrating agent such as corn starch, potato starch, alginic acid and the like; a lubricant such as magnesium stearate; or a sweetening agent such as sucrose, fructose, lactose or aspartame or a flavoring agent.

The active compound may also be administered intravenously or intraperitoneally by infusion or injection. Solutions of the active compound or its salts can be prepared in water, optionally mixed with a nontoxic surfactant.

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation can be vacuum drying and the freeze drying techniques, which can yield a powder of the active ingredient plus any additional desired ingredient present in the previously sterile-filtered solutions.

Exemplary solid carriers can include finely divided solids such as talc, clay, microcrystalline cellulose, silica, alumina and the like. Useful liquid carriers include water, alcohols or glycols or water-alcohol/glycol blends, in which the compounds or pharmaceutically acceptable salts thereof as described herein can be dissolved or dispersed at effective levels, optionally with the aid of non-toxic surfactants.

Useful dosages of a compound or pharmaceutically acceptable salt thereof as described herein can be determined by comparing their in vitro activity, and in vivo activity in animal models. Methods for the extrapolation of effective dosages in mice, and other animals, to humans are known to the art; for example, see U.S. Pat. No. 4,938,949, which is incorporated by reference in its entirety.

The amount of a compound or pharmaceutically acceptable salt thereof as described herein, required for use in treatment can vary not only with the particular salt selected but also with the route of administration, the nature of the condition being treated and the age and condition of the patient and can be ultimately at the discretion of the attendant physician or clinician. In general, however, a dose can be in the range of from about 0.1 to about 10 mg/kg of body weight per day.

The a compound or pharmaceutically acceptable salt thereof as described herein can be conveniently administered in unit dosage form; for example, containing 0.01 to 10 mg, or 0.05 to 1 mg, of active ingredient per unit dosage form. In some embodiments, a dose of 5 mg/kg or less can be suitable.

The desired dose may conveniently be presented in a single dose or as divided doses administered at appropriate intervals.

The disclosed method can include a kit comprising a compound or pharmaceutically acceptable salt thereof as described herein and instructional material which can describe administering a compound or pharmaceutically acceptable salt thereof as described herein or a composition comprising a compound or pharmaceutically acceptable salt thereof as described herein to a cell or a subject. This should be construed to include other embodiments of kits that are known to those skilled in the art, such as a kit comprising a (such as sterile) solvent for dissolving or suspending a compound or pharmaceutically acceptable salt thereof as described herein or composition prior to administering a compound or pharmaceutically acceptable salt thereof as described herein or composition to a cell or a subject. In some embodiments, the subject can be a human.

The invention is illustrated by the following examples, which are not intended to be limiting.

### EXEMPLIFICATION

Abbreviations and acronyms used herein include the following:
ABPR means automated back pressure regulator;
Ac₂O means acetic anhydride;
ACN means acetonitrile;
Aq. means aqueous;
Ar means argon;
Bn means benzyl;
Boc means tert-butoxy carbonyl;
Boc₂O means di-tert-butyl decarbonate;
BPin means pinacolatoboron;
B₂pin₂ means bispinacolatodiboron;
br means broad;
t-BuOH means tert butanol;
n-BuLi means n-butyl lithium;
° C. means degrees Celsius;
CHCl₃ means chloroform;
CDCl₃ means deutero-chloroform;
CO₂ means carbon dioxide;
Cs₂CO₃ means cesium carbonate;
CsF means cesium fluoride;
CuI means copper iodide;
S means chemical shift;
d means doublet;
dd means double doublet;
ddd means double doublet of doublets;
DCM means dichloromethane;
DIEA or DIPEA means N-ethyldiisopropylamine or N,N-diisopropylethylamine;
DEA means diethylamine;
deg means degrees;
DIAD means diisopropyl azodicarboxylate;
DME means 1,2-dimethoxyethane;
DMF means N,N-dimethylformamide;
DMSO means dimethylsulfoxide;
DMSO-d₆ means hexadeuterodimethyl sulfoxide;
DPPA means diphenylphosphoryl azide;
Et means ethyl;
Et₂O means ether;
EtOH means ethanol;
EtOAc means ethyl acetate;
Eq. means equivalent;
g means gram;
h means hour;
HATU means O-(7-azabenzotriazole-1-yl)-1,1,3,3-tetramethyluronium hexafluorophosphate;
HBr means hydrogen bromide;
HCl means hydrochloric acid;
HCO₂H means formic acid;
Hept means heptanes;
HFIP means hexafluoroisopropanol;
¹H NMR means proton nuclear magnetic resonance;
H₂O means water;
H₂SO₄ means sulfuric acid;
HMPA means hexamethylphosphoramide;
HPLC means high pressure liquid chromatography;
Hz means Hertz;
IPA or iPrOH means iso-propanol;
J means coupling constant;
K₂CO₃ means potassium carbonate;
kg means kilogram;
KHMDS means potassium hexamethldisilazide;
KOAc means potassium acetate;
KOH means potassium hydroxide;
KOt-Bu means potassium tert-butoxide
K₃PO₄ means potassium phosphate tribasic;
K₄Fe(CN)₆·3H₂O means potassium hexacyanoferrate (II) trihydrate;
L means liter;
LCMS means liquid chromatography mass spectrometry;
m means multiplet;
M means molar;
MBPR means manual back pressure regulator;
Me means methyl;
MeB(OH)₂ means methylboronic acid;
MeCN means acetonitrile;
MeOH means methanol;
MeOH-d₄ means deutero-methanol;
mg means milligram;
MgSO₄ means magnesium sulfate;
MHz means mega Hertz;
mins means minutes;
mL means milliliters;
mmol means millimole;
MMPNO means methylmorpholine N-oxide;
mol means mole;
MS m/z means mass spectrum peak;
N₂ means nitrogen;
NaOt-Bu means sodium tert-butoxide;
NaH means sodium hydride;
NaHCO₃ means sodium bicarbonate;
NaHMDS means sodium hexamethyldisilylazide;
NaIO₄ means sodium periodate;
NaOH means sodium hydroxide;
Na₂S₂O₃ means sodium thiosulfate;
Na₂SO₄ means sodium sulfate;
NEt₃ means triethylamine;
NFSI means N-fluorobenzenesulfonimide;
NH₃ means ammonia;
NH₄Cl means ammonium chloride;
NH₄OH is ammonium hydroxide;
NH₄OAc is ammonium acetate;
NIS means N-iodosuccinimide;
OsO₄ means osmium tetroxide;
P(cy)₃ means tricyclohexylphosphine;
Pd₂(dba)₃ means tris(dibenzylideneacetone)dipalladium (0);
Pd(dppf)Cl₂ means [1,1′-bis(diphenylphosphino)ferrocene]dichloropalladium(II);
Pd(dtbpf)Cl₂ means [1,1′-bis(di-tert-butylphosphino)ferrocene]dichloropalladium(II);
PEPPSI-IPr or Pd-PEPPSI-IPr means [1,3-bis(2,6-diisopropylphenyl)imidazol-2-ylidene](3-chloropyridyl)palladium(II) dichloride
Ph means phenyl;
POCl₃ means phosphoryl chloride;
Pyr means pyridine;
q means quartet;
Rf means retardation factor;
Rt means retention time;
RT means room temperature;
RuPhos means 2-dicyclohexylphosphino-2′,6′-diisopropoxybiphenyl;
s means singlet;
sat. means saturated;
SCX means strong cation exchange;
SFC means supercritical fluid chromatography;
SiO₂ means silicon dioxide;
Si-SPE means silica solid phase extraction;
t means triplet;
td means triple doublet;
t-BuONa means sodium tert-butoxide;
TEA means triethylamine;
TFA means trifluoroacetic acid;
THF means tetrahydrofuran;
TLC means thin layer chromatography;
T₃P means propanephosphonic acid anhydride;
μL means microliters;
μmol means micromole;
ρW means microwave;
v/v means volume per volume;
Xphos means 2-dicyclohexylphosphino-2′,4′,6′-triisopropylbiphenyl;
Xphos G3 means (2-dicyclohexylphosphino-2′,4′,6′-triisopropyl-1,1′-biphenyl)[2-(2′-amino-1,1′-biphenyl)]palladium(II) methanesulfonate.

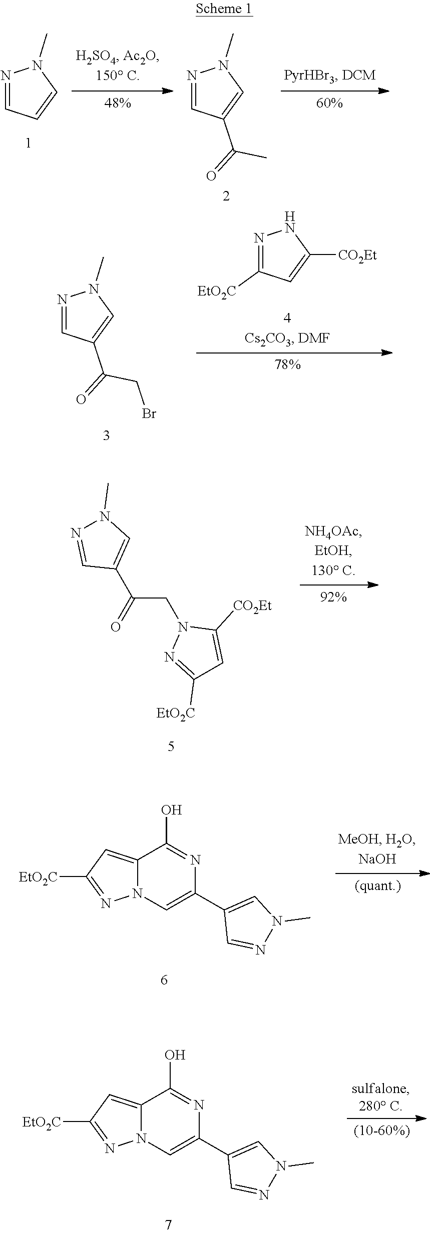

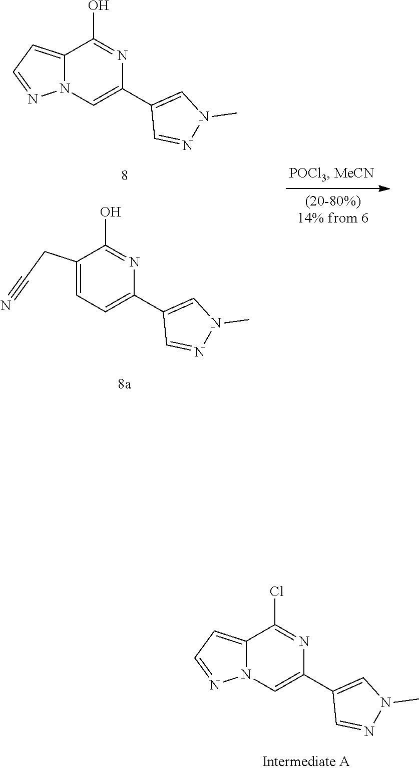 [SMILES: Cn1cc(-c2cn3nccc3c(O)n2)cn1 ; Cn1cc(-c2ccc(CC#N)c(O)n2)cn1 ; Cn1cc(-c2cn3nccc3c(Cl)n2)cn1]

### Synthesis of 1-(1-methyl-1H-pyrazol-4-yl)ethan-1-one (2)

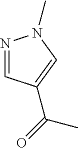

To a mixture of 1-methyl-1H-pyrazole (50.0 g, 1 eq., 609 mmol) and acetic anhydride (112 g, 104 mL, 1.8 eq., 1.10 mol) was added sulfuric acid (4.78 g, 2.61 mL, 0.08 eq., 48.7 mmol) at room temperature. The mixture was heated at 150° C. for 7 h after which it was allowed to cool to RT overnight. The reaction mixture was poured into ice, the pH of the resulting solution was adjusted to 10 with a 20% solution of NaOH in water and then extracted with DCM, the organic phase was dried over sodium sulfate and concentrated. This gave 1-(1-methyl-1H-pyrazol-4-yl)ethan-1-one (36.8 g, 49% yield). ¹H NMR (300 MHz, CDCl₃) δ 7.80-7.96 (m, 2H), 3.91 (s, 3H), 2.39 (s, 3H).

### Synthesis of 2-bromo-1-(1-methyl-1H-pyrazol-4-yl)ethan-1-one (3)

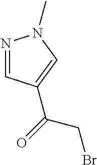

In a round bottom flask, 1-(1-methyl-1H-pyrazol-4-yl)ethan-1-one (36.8 g, 1 eq., 296 mmol) was dissolved in dichloromethane (700 mL). Ethanol (175 mL) and pyridinium tribromide (94.7 g, 1 eq., 296 mmol) were added portion-wise at 15° C. The mixture was stirred from 0° C. to room temperature overnight. The mixture was checked by TLC (heptanes: EtOAc 4:6) and HPLC. After complete addition, the reaction was quenched with water. The layers were separated, and the organic phase was dried over sodium sulfate and concentrated to give the product as a brown solid. The solid was suspended in a mixture of DCM and heptane, warmed to 50° C. and cooled to room temperature again. The product precipitated and was isolated by filtration (28.8 g). From the mother liquor more solid precipitated (7.43 g). In total, 36.2 g (60% yield) of the titled product was isolated as a brown solid. ESI-MS (M+H)⁺: 205.1.

### Synthesis of diethyl 1-(2-(1-methyl-1H-pyrazol-4-yl)-2-oxoethyl)-1H-pyrazole-3,5-dicarboxylate (5)

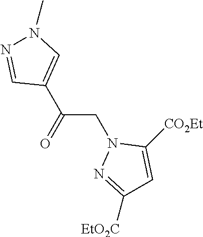 [SMILES: CCOC(=O)c1cc(C(=O)OCC)n(CC(=O)c2cnn(C)c2)n1]

In a round bottom flask, 2-bromo-1-(1-methyl-1H-pyrazol-4-yl)ethan-1-one (60.6 g, 1 eq., 298 mmol) was dissolved in DMF (900 mL), and diethyl 1H-pyrazole-3,5-dicarboxylate (69.6 g, 1.1 eq., 328 mmol) and cesium carbonate (126 g, 1.30 eq., 388 mmol) were added. The reaction was stirred at room temperature overnight. The reaction mixture was diluted with water and extracted with DCM. The organic layer was dried over sodium sulfate and concentrated. The crude product was suspended in heptanes: EtOAc 1:1 (50-100 mL) and filtrated. The solid was washed once with EtOAc and once with heptanes to give the product (68.5 g) as a white solid. The mother liquor was concentrated and purified by column chromatography (120 g silica, heptanes: EtOAc gradient 0 to 100%) to give another portion of the product (9.7 g). In total, 78.2 g (78% yield) of product was isolated as a white solid. ESI-MS (M+H)⁺: 335.2.

### Synthesis of ethyl 4-hydroxy-6-(1-methyl-1H-pyrazol-4-yl)pyrazolo[1,5-a]pyrazine-2-carboxylate (6)

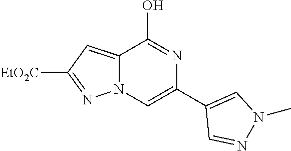 [SMILES: CCOC(=O)c1cc2c(O)nc(-c3cnn(C)c3)cn2n1]

A Berghoff reactor was charged with diethyl 1-(2-(1-methyl-1H-pyrazol-4-yl)-2-oxoethyl)-1H-pyrazole-3,5-dicarboxylate (15.0 g, 1 eq., 44.9 mmol), ethanol (150 mL) and ammonium acetate (10.4 g, 3.0 eq., 135 mmol). The mixture was heated at 130° C. for 24 h, after which HPLC revealed complete conversion (a sample was taken after cooling the reactor to RT again). The reaction mixture was filtered off, washed with water and dried in air to give the product (11.8 g, 92%) as a white solid. This reaction was performed batchwise on a total amount of 78.2 g of starting material, to give a total amount of 66.6 g of product (92% yield). ESI-MS (M+H)⁺: 288.3.

### Synthesis of 4-hydroxy-6-(1-methyl-1H-pyrazol-4-yl)pyrazolo[1,5-a]pyrazine-2-carboxylic acid (7)

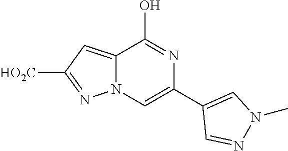 [SMILES: Cn1cc(-c2cn3nc(C(=O)O)cc3c(O)n2)cn1]

In a round bottom flask, ethyl 4-hydroxy-6-(1-methyl-1H-pyrazol-4-yl)pyrazolo[1,5-a]pyrazine-2-carboxylate (66.6 g, 1.0 eq., 232 mmol) was suspended in methanol (1.2 L), and 1M sodium hydroxide (27.9 g, 696 mL, 3.0 eq., 696 mmol) was added at room temperature. The mixture was stirred at room temperature overnight. The mixture was acidified to pH 2 with conc. HCl, then filtrated (filtration very slow and difficult). The solid was washed with MeOH, transferred to a round bottom flask and stripped with acetonitrile. The obtained product turned out to be a mixture of the methyl ester and salts (92.8 g, max. 232 mmol). The solid was split in two portions and the hydrolysis was repeated. In a round bottom flask methyl 4-hydroxy-6-(1-methyl-1H-pyrazol-4-yl)pyrazolo[1,5-a]pyrazine-2-carboxylate (46.0 g, 1.0 eq., 116 mmol) was suspended in methanol (1.2 L) and 1 M sodium hydroxide (13.9 g, 348 mL, 3.0 eq., 348 mmol) and 10 ml of water were added at room temperature. The mixture was stirred at room temperature overnight. The mixture was neutralized to pH 7 with conc. HCl, then filtrated (filtration still difficult). The solid was washed with acetonitrile, dioxane, transferred to a round bottom flask and stripped with acetonitrile to give the first batch of 4-hydroxy-6-(1-methyl-1H-pyrazol-4-yl)pyrazolo[1,5-a]pyrazine-2-carboxylic acid Batch 1 (71.0 g, max. 116 mmol, Batch 1) containing a lot of salts. The same was repeated on the second batch of the methyl ester. In this case when conversion was complete the reaction mixture was acidified to pH 5. This gave 4-hydroxy-6-(1-methyl-1H-pyrazol-4-yl)pyrazolo[1,5-a]pyrazine-2-carboxylic acid (60.0 g, max. 116 mmol, Batch 1) with a lot of salts. ESI-MS (M−H)⁺: 258.0.

### Synthesis of 6-(1-methyl-1H-pyrazol-4-yl)pyrazolo[1,5-a]pyrazin-4-ol (8)

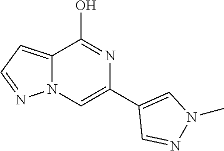 [SMILES: Cn1cc(-c2cn3nccc3c(O)n2)cn1]

A three-necked flask was charged with pre-heated sulfolane (0.24 kg, 0.19 L, 30 eq., 2.0 mol), which was heated to 50° C. Then 4-hydroxy-6-(1-methyl-1H-pyrazol-4-yl)pyrazolo[1,5-a]pyrazine-2-carboxylic acid (41.8 g, 1.0 eq., 68 mmol) and few drops of conc sulfuric acid were added portion-wise. The reaction mixture was heated at 350° C. (external, gentle reflux of sulfolane) and conversion was checked every hour. After 4 h the reaction mixture was cooled to room temperature, diluted with DCM and purified by filtration over a short plug of silica eluting with 3L of heptanes (fr1), 6L heptanes EtOAc 1:1 (fr2-3), 6L EtOAc (fr4-5), 4 L DCM (fr6), 6 L DCM: MeOH 9:1 (fr7-8). The product (containing side-product 8a) (2.88 g, 20%) was isolated from fr7 as a brown solid. ESI-MS (M−H)+: 214.1.

### Synthesis of 4-chloro-6-(1-methyl-1H-pyrazol-4-yl)pyrazolo[1,5-a]pyrazine

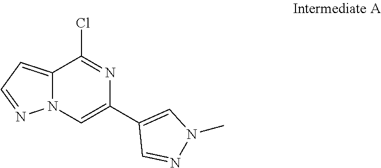 [SMILES: Cn1cc(-c2cn3nccc3c(Cl)n2)cn1]

In a round bottom flask, 6-(1-methyl-1H-pyrazol-4-yl)pyrazolo[1,5-a]pyrazin-4-ol (2.88 g, 1.0 eq., 13.4 mmol) was suspended in POCl₃ (32.8 g, 19.9 mL, 16 eq., 214 mmol) and the reaction was heated at 80° C. overnight. The mixture was diluted with acetonitrile and concentrated, the residue was suspended in DCM and the mixture was washed with sat. NaHCO₃ and brine, dried over sodium sulfate and concentrated. The crude product was purified by column chromatography (DCM: EtOAc/NEt₃ 5% gradient 0 to 25%) to give the product (1.35 g, 43%) as a yellow solid. ESI-MS (M+H)⁺: 234.0.

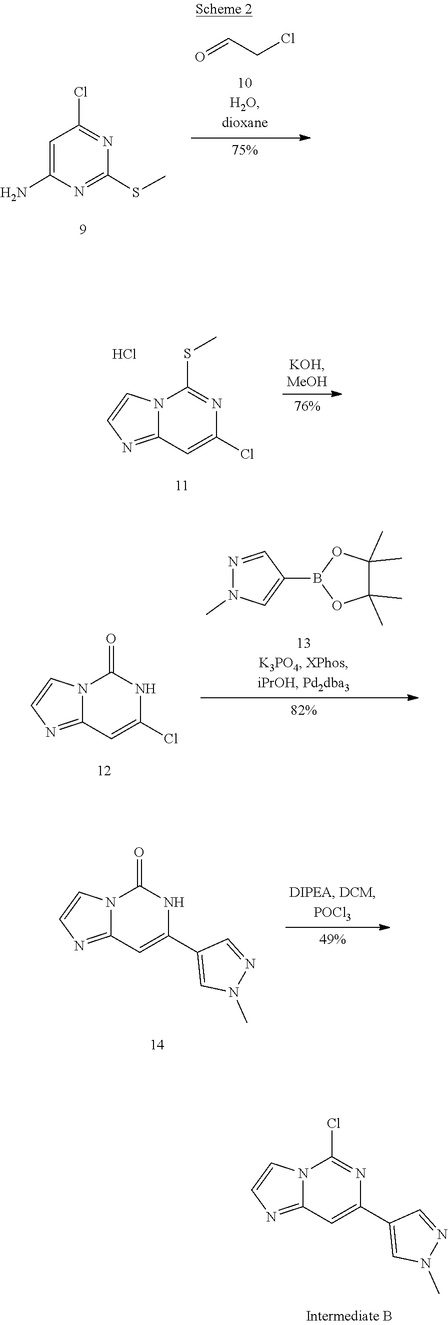

### Synthesis of 7-chloro-5-(methylthio)imidazo[1,2-c]pyrimidine hydrochloride (11)

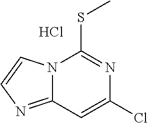

In a round bottom flask, 6-chloro-2-(methylthio)pyrimidin-4-amine (150 g, 1 eq., 854 mmol) was dissolved in 1,4-dioxane (300 mL) and 2-chloroacetaldehyde (220 g, 0.18 L, 1.5 eq., 1.28 mol) was added. The mixture was stirred at 100° C. After 2 h a solid precipitated in the reaction mixture and after 3 h the reaction was checked by HPLC to reveal that conversion as completed. The reaction mixture was cooled to RT overnight. The suspension was cooled to 0° C. and the solid was filtered off to give the product (151 g, 75%) as a yellow solid. ESI-MS (M+H)⁺: 200.1.

### Synthesis of 7-chloroimidazo[1,2-c]pyrimidin-5(6H)-one (12)

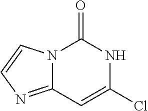 [SMILES: O=c1[nH]c(Cl)cc2nccn12]

In a 3-necked flask 7-chloro-5-(methylthio)imidazo[1,2-c]pyrimidine hydrochloride (52.2 g, 1 eq., 221 mmol) was suspended in MeOH (200 mL). A solution of potassium hydroxide (55.9 g, 4.5 eq., 996 mmol) in water (520 mL) was added slowly. The reaction was heated at reflux for 3 h and then checked by HPLC-MS was checked. Starting material disappeared. The reaction was cooled to room temperature overnight. The mixture was acidified to pH 6 with 1M HCl and the obtained suspension was filtrated. The solid was washed with MeOH then transferred in a round bottom flask and suspended in ACN, then concentrated. Product 7-chloroimidazo[1,2-c]pyrimidin-5(6H)-one (28.55 g, 76%) was obtained clean as a white solid. ESI-MS (M+H)⁺: 170.1.

### Synthesis of 7-(1-methyl-1H-pyrazol-4-yl)imidazo[1,2-c]pyrimidin-5(6H)-one (13)

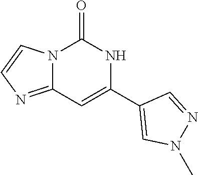 [SMILES: Cn1cc(-c2cc3nccn3c(=O)[nH]2)cn1]

In a 3-necked flask, 7-chloroimidazo[1,2-c]pyrimidin-5(6H)-one (40.0 g, 1 eq., 236 mmol), 1-methyl-4-(4,4,5,5-tetramethyl-1,3,2-dioxaborolan-2-yl)-1H-pyrazole (73.6 g, 1.5 eq., 354 mmol) and X-phos (11.2 g, 0.10 eq., 23.6 mmol) were dissolved in 2-propanol (1.8 L) and a 2 M solution of potassium phosphate (150 g, 0.35 L, 3.0 eq., 708 mmol) in water was added. The mixture was purged with N₂ for 15 min then Pd₂(dba)₃ (10.8 g, 0.05 eq., 11.8 mmol) was added and the mixture was refluxed overnight. The reaction was checked by HPLC-MS and analysis showed that conversion was almost complete. Pd₂(dba)₃ (5.0 g) and 1-methyl-4-(4,4,5,5-tetramethyl-1,3,2-dioxaborolan-2-yl)-1H-pyrazole (25.0 g) were added and the mixture refluxed for one more night. HPLC-MS showed complete conversion. The reaction mixture was filtered to remove palladium-residues. The organic solvent was evaporated, and the residue was partitioned between water and a 1:1-mixture of heptanes: EtOAc. A white solid precipitated in both the organic and aqueous layer: the mixture was filtered off. The solid was washed with water, ethyl acetate and acetonitrile and dried in vacuo to give the product (32.8 g). The layers of the filtrate were separated. The organic phase was discarded, the aqueous layer was cooled on an ice bath. The solution was treated with concentrated HCl to pH 6 with stirring and the resulting fine precipitate was collected, washed with H₂O and Et₂O and dried under vacuum to give another portion of the product (9.0 g). In total 41.8 g (82%) of product was obtained as a yellowish solid. ESI-MS (M+H)⁺: 215.0.

### Synthesis of 5-chloro-7-(1-methyl-1H-pyrazol-4-yl)imidazo[1,2-c]pyrimidine

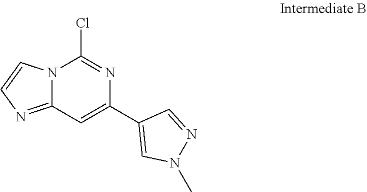 [SMILES: Cn1cc(-c2cc3nccn3c(Cl)n2)cn1]

A round-bottomed flask was charged with 7-(1-methyl-1H-pyrazol-4-yl)imidazo[1,2-c]pyrimidin-5(6H)-one (41.8 g, 1 eq., 194 mmol), dry DCM (300 mL) and DIPEA (126 g, 0.17 L, 5 eq., 971 mmol). After 5 min the mixture was cooled to 0° C. and POCl₃ (89.3 g, 54.1 mL, 3 eq., 583 mmol) was added dropwise over 5 min. The mixture was brought to room temperature and diluted with DCM (150 mL), then stirred for 24 h at room temperature. The suspension was diluted with hexanes and the solid was collected by filtration (66.0 g). The collected solid was suspended in DCM:DIPEA (5:1, 500 mL). The mixture was stirred for 30 min and then a saturated aqueous solution of NaHCO₃ was added and the mixture was stirred for 1 h. The mixture was filtered over Celite, then the layers were separated, and the aqueous layer was extracted 3 times with DCM. The organic layers were dried over sodium sulfate and concentrated. The product 5-chloro-7-(1-methyl-1H-pyrazol-4-yl)imidazo[1,2-c]pyrimidine (22.5 g, 50%) was obtained as a yellow solid. ESI-MS (M+H)⁺: 234.0.

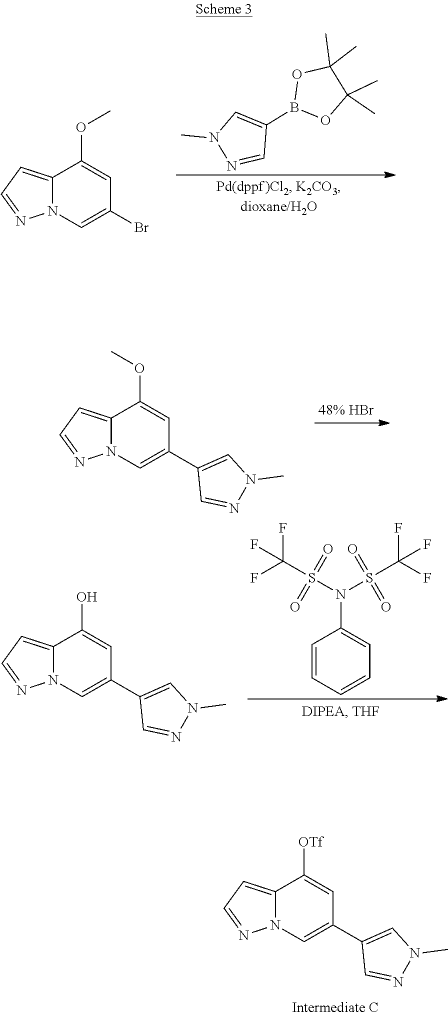

### Synthesis of 4-methoxy-6-(1-methyl-1H-pyrazol-4-yl)pyrazolo[1,5-a]pyridine

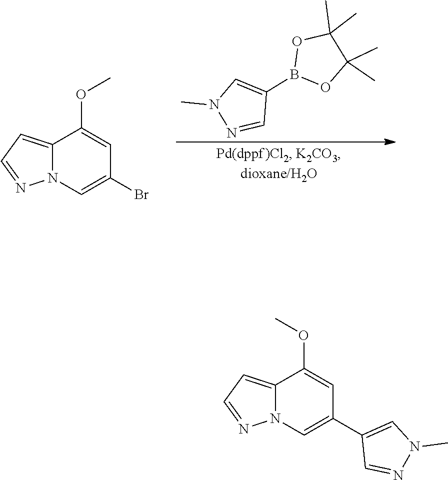

To a solution of 6-bromo-4-methoxypyrazolo[1,5-a]pyridine (8.0 g, 35 mmol) and 1-methyl-4-(4,4,5,5-tetramethyl-1,3,2-dioxaborolan-2-yl)-1H-pyrazole (8.8 g, 42 mmol) in water (30 mL) and dioxane (150 mL) were added K₂CO₃(9.74 g, 70.5 mmol) and Pd(dppf)Cl₂ (1.29 g, 1.76 mmol) and the reaction mixture was stirred at 90° C. for 2 h under N₂. The reaction mixture was diluted with H₂O (80 mL) and extracted with EtOAc (100 mL×2). The combined organic phase was dried over Na₂SO₄ and filtered. The filtrate was concentrated in vacuo and the residue was purified by column chromatography on silica gel (PE/EtOAc=1/1-0/1) to give 4-methoxy-6-(1-methyl-1H-pyrazol-4-yl)pyrazolo[1,5-a]pyridine (8.0 g) as a white solid. 1HNMR (400 MHz, CDCl₃) δ: 8.27 (s, 1H), 7.86 (d, J=1.6 Hz, 1H), 7.74 (s, 1H), 7.61 (s, 1H), 6.62 (s, 1H), 6.47 (s, 1H), 4.00 (s, 3H), 3.97 (s, 3H)

### 1. Synthesis of 6-(1-methyl-1H-pyrazol-4-yl)pyrazolo[1,5-a]pyridin-4-ol

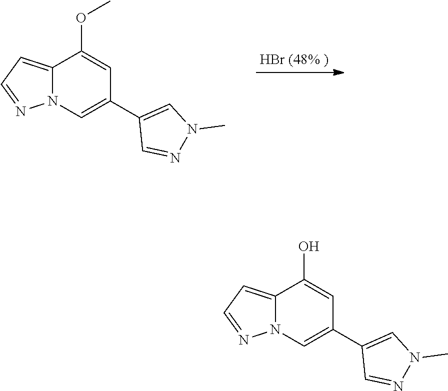 [SMILES: COc1cc(-c2cnn(C)c2)cn2nccc12 ; Cn1cc(-c2cc(O)c3ccnn3c2)cn1]

A solution of 4-methoxy-6-(1-methyl-1H-pyrazol-4-yl)pyrazolo[1,5-a]pyridine (15 g, 65.7 mmol) in aq. HBr (100 mL, 48%) was stirred at 120° C. for 48 h. The reaction mixture was concentrated in vacuo, the residue quenched with sat. NaHCO₃ until pH to 8 and extracted with EtOAc (3×80 mL). The combined organic layer was dried over Na₂SO₄ and filtered. The filtrate was concentrated to give the crude product, which was purified by column chromatography on silica gel (DCM/MeOH=20/1-10/1) to give 6-(1-methyl-1H-pyrazol-4-yl)pyrazolo[1,5-a]pyridin-4-ol (13.0 g, 92% yield) as a grey solid. LCMS m/z=215.0 (M+H)+

### 2. Synthesis of 6-(1-methyl-1H-pyrazol-4-yl)pyrazolo[1,5-a]pyridin-4-yl trifluoromethanesulfonate

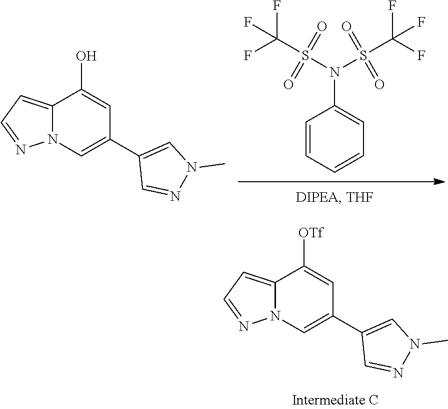 [SMILES: Cn1cc(-c2cc(O)c3ccnn3c2)cn1 ; O=S(=O)(N(c1ccccc1)S(=O)(=O)C(F)(F)F)C(F)(F)F ; Cn1cc(-c2cc(OS(=O)(=O)C(F)(F)F)c3ccnn3c2)cn1]

To a solution of 6-(1-methyl-1H-pyrazol-4-yl)pyrazolo[1,5-a]pyridin-4-ol (40 g, 136 mmol) in THF (600 mL) was added DIPEA (87.58 g, 678 mmol) and N-phenyl-bis(trifluoromethanesulfonimide) (72.63 g, 203 mmol) and the reaction stirred at 20° C. for 20 h. The reaction mixture was diluted with H₂O (500 mL) and extracted with EtOAc (3×350 mL). The combined organic layer was dried over Na₂SO₄ and filtered. The filtrate was concentrated in vacuo and the crude purified by column chromatography on silica gel (PE/EtOAc=20/1-1/1) to give 6-(1-methyl-1H-pyrazol-4-yl)pyrazolo[1,5-a]pyridin-4-yl trifluoromethanesulfonate (32.0 g, 68% yield) as a yellow solid and a further 10 g, crude. LCMS m/z=347.1 (M+H)+

### C. Synthesis of Examples 1-236

### Example 1: 1-[4-[6-(1-methylpyrazol-4-yl)pyrazolo[1,5-a]pyrazin-4-yl]oxy-1-piperidyl]prop-2-yn-1-one

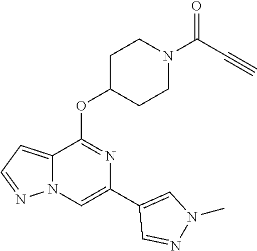 [SMILES: C#CC(=O)N1CCC(Oc2nc(-c3cnn(C)c3)cn3nccc23)CC1]

### Synthesis of tert-butyl 4-[6-(1-methylpyrazol-4-yl)pyrazolo[1,5-a]pyrazin-4-yl]oxypiperidine-1-carboxylate

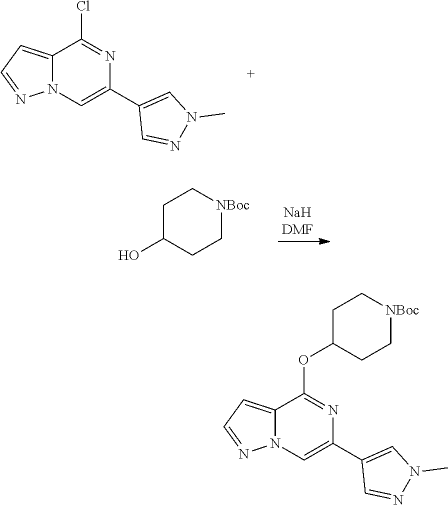

A solution of tert-butyl 4-hydroxypiperidine-1-carboxylate (664 mg, 3.30 mmol) in dry DMF (10 mL) was cooled in an ice bath. Then, sodium hydride (396 mg, 9.90 mmol, 60% purity) was added in 4 batches while stirring. Stirring in the ice bath was continued for 45 minutes during which a pale-yellow suspension formed. To this mixture was added 4-chloro-6-(1-methylpyrazol-4-yl)pyrazolo[1,5-a]pyrazine (701 mg, 3.00 mmol) as one batch and the mixture turned instantly brown orange. Stirring at RT was continued overnight. The mixture was diluted with EtOAc and then water was added carefully. The mixture was transferred to a separating funnel and the phases were separated. The aq. phase was extracted with EtOAc once more and the combined organic phases were washed with brine, dried with Na₂SO₄, filtered and evaporated in vacuo. The residual material was purified on a 10 g Si-SPE column: Rt=0.18 in heptanes/EtOAc=1/1 to give tert-butyl 4-[6-(1-methylpyrazol-4-yl)pyrazolo[1,5-a]pyrazin-4-yl]oxypiperidine-1-carboxylate (1.30 g, 98% yield, 90% purity) as a sticky yellow gum. LCMS: m/z=399.0 (M+H⁺).

### Synthesis of 6-(1-methylpyrazol-4-yl)-4-(4-piperidyloxy)pyrazolo[1,5-a]pyrazine

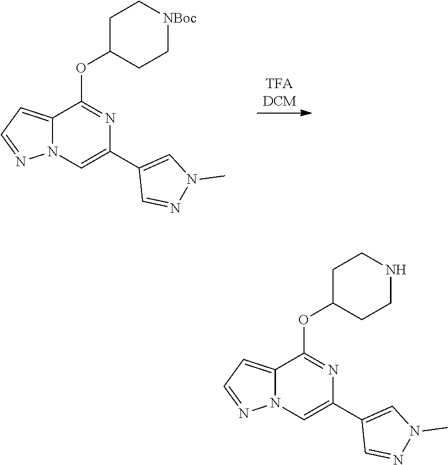 [SMILES: Cn1cc(-c2cn3nccc3c(OC3CCN(C(=O)OC(C)(C)C)CC3)n2)cn1 ; Cn1cc(-c2cn3nccc3c(OC3CCNCC3)n2)cn1]

To a solution of tert-butyl 4-[6-(1-methylpyrazol-4-yl)pyrazolo[1,5-a]pyrazin-4-yl]oxypiperidine-1-carboxylate (1.17 g, 2.94 mmol) in DCM (5 mL) was added TFA (6.70 g, 58.8 mmol, 4.5 mL) with stirring at RT. Stirring was continued overnight. The mixture was diluted with MeOH and purified on a 10 g SCX column where the product eluted with 2 M NH₃-MeOH to give 6-(1-methylpyrazol-4-yl)-4-(4-piperidyloxy)pyrazolo[1,5-a]pyrazine (890 mg, 96% yield, 95% purity) as a pale-yellow solid. LCMS: m/z=299.0 (M+H⁺).

### Synthesis of 1-[4-[6-(1-methylpyrazol-4-yl)pyrazolo[1,5-a]pyrazin-4-yl]oxy-1-piperidyl]prop-2-yn-1-one

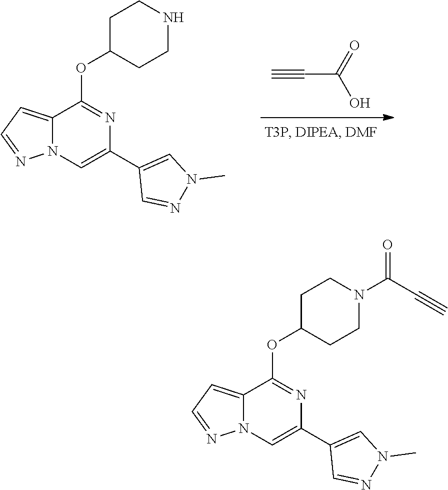

To a solution of 6-(1-methylpyrazol-4-yl)-4-(4-piperidyloxy)pyrazolo[1,5-a]pyrazine (30 mg, 101 μmol) in DMF (1 mL) and propiolic acid (7.0 mg, 101 μmol, 6 μL) was added DIPEA (26 mg, 201 μmol, 35 μL) with stirring at RT. Then, T3P (128 mg, 201 μmol, 50% purity) was added with stirring. Stirring was continued overnight. The mixture was diluted with EtOAc and washed with water. The organic phase was dried with Na2SO4, filtered and the filtrate was evaporated to dryness. This material was dissolved in DMSO, filtered through a syringe filter and purified with prep HPLC (Waters XSelect CSH C18, 5 μm, 19 mm×100 mm column with mobile phase H₂O (A) and MeCN (B) and a gradient of 5-50% B (0.2% NH₄OH final v/v % modifier) with flow rate at 30 mL/min) to give 1-[4-[6-(1-methylpyrazol-4-yl)pyrazolo[1,5-a]pyrazin-4-yl]oxy-1-piperidyl]prop-2-yn-1-one (12.6 mg, 95% purity, 34% yield) as a white solid. LCMS: m/z=351.0 (M+H⁺). ¹H NMR (500 MHz, DMSO-d6) δ 8.76 (s, 1H), 8.20 (s, 1H), 7.98-8.04 (m, 2H), 6.83-6.92 (m, 1H), 5.55-5.69 (m, 1H), 4.57 (s, 1H), 3.95-4.06 (m, 1H), 3.88 (s, 3H), 3.72-3.86 (m, 2H), 3.50-3.61 (m, 1H), 2.12-2.20 (m, 1H), 2.02-2.11 (m, 1H), 1.82-1.91 (m, 1H), 1.73-1.81 (m, 1H).

### Example 2: 1-(4-((6-(1-methyl-1H-pyrazol-4-yl)pyrazolo[1,5-a]pyrazin-4-yl)oxy)piperidin-1-yl)prop-2-en-1-one

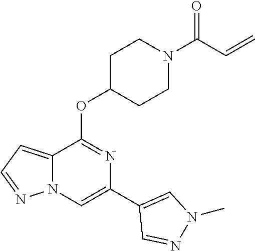 [SMILES: C=CC(=O)N1CCC(Oc2nc(-c3cnn(C)c3)cn3nccc23)CC1]

A 20 mL screw top vial was charged with 6-(1-methylpyrazol-4-yl)-4-(4-piperidyloxy)pyrazolo[1,5-a]pyrazine (30 mg, 101 μmol) and THF (1 mL). Then, acryloyl chloride (12 μL, 151 μmol) was added with stirring upon which a milky suspension formed instantaneously. Then, triethylamine (28 μL, 201 μmol) was added with stirring. After 5 minutes stirring at RT the volatiles were evaporated and left behind a white solid. This material was dissolved in DMSO, filtered through a syringe filter and purified with prep HPLC (Waters XSelect CSH C18, 5 μm, 19 mm×100 mm column with mobile phase H₂O (A) and MeCN (B) and a gradient of 5-50% B (0.2% NH₄OH final v/v % modifier) with flow rate at 30 mL/min) to give 1-(4-((6-(1-methyl-1H-pyrazol-4-yl)pyrazolo[1,5-a]pyrazin-4-yl)oxy)piperidin-1-yl)prop-2-en-1-one (24.9 mg, 95% purity, 67% yield) as a white solid. LCMS: m/z=353.0 (M+H⁺). ¹H NMR (500 MHz, DMSO-d₆) δ 8.75 (d, J=1.22 Hz, 1H), 8.21 (s, 1H), 8.01-8.03 (m, 2H), 6.86-6.88 (m, 1H), 6.83-6.90 (m, 1H), 6.12 (br dd, J=2.44, 16.48 Hz, 1H), 5.67-5.73 (m, 1H), 5.60 (ddd, J=3.97, 7.63, 11.60 Hz, 1H), 3.85-3.97 (m, 2H), 3.89 (s, 3H), 3.39-3.69 (m, 2H), 1.98-2.21 (m, 2H), 1.59-1.91 (m, 2H).

### Example 3: 6-(1-methyl-1H-pyrazol-4-yl)-4-((1-(vinylsulfonyl)piperidin-4-yl)oxy)pyrazolo[1,5-a]pyrazine

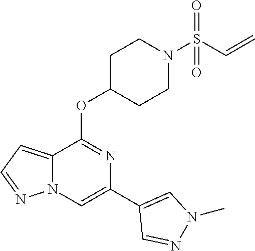 [SMILES: C=CS(=O)(=O)N1CCC(Oc2nc(-c3cnn(C)c3)cn3nccc23)CC1]

6-(1-methyl-1H-pyrazol-4-yl)-4-((1-(vinylsulfonyl)piperidin-4-yl)oxy)pyrazolo[1,5-a]pyrazine was prepared in a way similar to Example 2 except replaced acryloyl chloride with 2-chloro-ethane-sulfonyl chloride. The material was purified with prep HPLC (Waters XSelect CSH C18, 5 μm, 19 mm×100 mm column with mobile phase H₂O (A) and MeCN (B) and a gradient of 5-55% B (0.2% NH₄OH final v/v % modifier) with flow rate at 30 mL/min) to give 6-(1-methyl-1H-pyrazol-4-yl)-4-((1-(vinylsulfonyl)piperidin-4-yl)oxy)pyrazolo[1,5-a]pyrazine (4.9 mg, 95% purity, 12% yield) as a white solid. LCMS: m/z=389.0 (M+H⁺). ¹H NMR (500 MHz, DMSO-d₆) δ 8.75 (d, J=1.22 Hz, 1H), 8.20 (s, 1H), 8.02 (d, J=2.44 Hz, 1H), 8.01 (s, 1H), 6.89 (dd, J=10.38, 16.48 Hz, 1H), 6.85 (d, J=3.05 Hz, 1H), 6.20 (d, J=9.77 Hz, 1H), 6.16 (d, J=17.09 Hz, 1H), 5.49 (ddd, J=3.66, 7.63, 11.29 Hz, 1H), 3.88 (s, 3H), 3.37-3.54 (m, 2H), 3.18 (m, 2H), 2.10-2.24 (m, 2H), 1.79-1.96 (m, 2H).

### Example 4: (R)-1-(3-((6-(1-methyl-1H-pyrazol-4-yl)pyrazolo[1,5-a]pyrazin-4-yl)oxy)piperidin-1-yl)prop-2-en-1-one

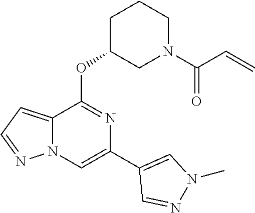 [SMILES: C=CC(=O)N1CCC[C@@H](Oc2nc(-c3cnn(C)c3)cn3nccc23)C1]

(R)-1-(3-((6-(1-methyl-1H-pyrazol-4-yl)pyrazolo[1,5-a]pyrazin-4-yl)oxy)piperidin-1-yl)prop-2-en-1-one was prepared in a way similar to Example 2 except starting with (R)-tert-butyl 3-hydroxypiperidine-1-carboxylate. The material was purified with prep HPLC (Waters XSelect CSH C18, 5 μm, 19 mm×100 mm column with mobile phase H₂O (A) and MeCN (B) and a gradient of 5-50% B (0.2% NH₄OH final v/v % modifier) with flow rate at 30 mL/min) to give (R)-1-(3-((6-(1-methyl-1H-pyrazol-4-yl)pyrazolo[1,5-a]pyrazin-4-yl)oxy)piperidin-1-yl)prop-2-en-1-one (23.1 mg, 95% purity, 67% yield) as a white powder. LCMS: m/z=353.0 (M+H⁺). ¹H NMR (500 MHz, DMSO-d₆) δ 8.76 (s, 1H), 8.28-8.33 (m, 1H), 8.14-8.22 (m, 1H), 7.97-8.03 (m, 1H), 6.67-6.77 (m, 1H), 6.52-6.97 (m, 1H), 5.92-6.15 (m, 1H), 5.42-5.74 (m, 1H), 5.19-5.40 (m, 1H), 3.88 (s, 3H), 3.59-4.27 (m, 4H), 1.47-2.22 (m, 4H).

### Example 5: (R)-6-(1-methyl-1H-pyrazol-4-yl)-4-((1-(vinylsulfonyl)piperidin-3-yl)oxy)pyrazolo[1,5-a]pyrazine

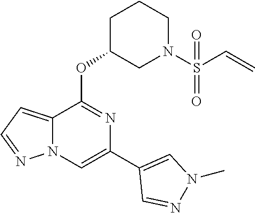 [SMILES: C=CS(=O)(=O)N1CCC[C@@H](Oc2nc(-c3cnn(C)c3)cn3nccc23)C1]

(R)-6-(1-methyl-1H-pyrazol-4-yl)-4-((1-(vinylsulfonyl)piperidin-3-yl)oxy)pyrazolo[1,5-a]pyrazine was prepared in a way similar to Example 3 except starting with (R)-tert-butyl 3-hydroxypiperidine-1-carboxylate. The material was purified with prep HPLC (Waters XSelect CSH C18, 5 μm, 19 mm×100 mm column with mobile phase H₂O (A) and MeCN (B) and a gradient of 5-55% B (0.2% NH₄OH final v/v % modifier) with flow rate at 30 mL/min) to give (R)-6-(1-methyl-1H-pyrazol-4-yl)-4-((1-(vinylsulfonyl)piperidin-3-yl)oxy)pyrazolo[1,5-a]pyrazine (8.9 mg, 95% purity, 22% yield) as a white powder. LCMS: m/z=389.0 (M+H⁺). ¹H NMR (500 MHz, DMSO-d₆) δ 8.78 (s, 1H), 8.21 (s, 1H), 8.03 (d, J=1.83 Hz, 1H), 8.02 (s, 1H), 6.84 (d, J=2.44 Hz, 1H), 6.78-6.88 (m, 1H), 6.11 (s, 1H), 6.08 (d, J=6.10 Hz, 1H), 5.38 (ddd, J=3.66, 7.17, 10.53 Hz, 1H), 3.88 (s, 3H), 3.74 (br dd, J=3.66, 12.21 Hz, 1H), 3.46 (br d, J=18.31 Hz, 1H), 3.21-3.29 (m, 1H), 3.11 (ddd, J=3.36, 8.09, 11.75 Hz, 1H), 2.05 (ddd, J=3.66, 8.39, 12.36 Hz, 1H), 1.89-1.98 (m, 1H), 1.79-1.88 (m, 1H), 1.71 (tdd, J=4.04, 8.47, 17.01 Hz, 1H).

### Example 6: (S)-6-(1-methyl-1H-pyrazol-4-yl)-4-((1-(vinylsulfonyl)piperidin-3-yl)oxy)pyrazolo[1,5-a]pyrazine

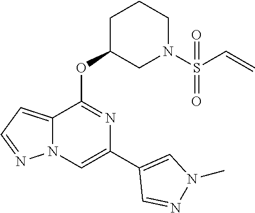 [SMILES: C=CS(=O)(=O)N1CCC[C@H](Oc2nc(-c3cnn(C)c3)cn3nccc23)C1]

(S)-6-(1-methyl-1H-pyrazol-4-yl)-4-((1-(vinylsulfonyl)piperidin-3-yl)oxy)pyrazolo[1,5-a]pyrazine was prepared in a way similar to Example 3 except starting with (S)-tert-butyl 3-hydroxypiperidine-1-carboxylate. The material was purified with prep HPLC (Waters XSelect CSH C18, 5 μm, 19 mm×100 mm column with mobile phase H₂O (A) and MeCN (B) and a gradient of 5-55% B (0.2% NH₄OH final v/v % modifier) with flow rate at 30 mL/min) to give (S)-6-(1-methyl-1H-pyrazol-4-yl)-4-((1-(vinylsulfonyl)piperidin-3-yl)oxy)pyrazolo[1,5-a]pyrazine (8.0 mg, 95% purity, 18% yield). LCMS: m/z=389.0 (M+H⁺). ¹H NMR (500 MHz, DMSO-d₆) δ 8.78 (s, 1H), 8.21 (s, 1H), 8.03 (d, J=1.83 Hz, 1H), 8.02 (s, 1H), 6.84 (d, J=2.44 Hz, 1H), 6.79-6.86 (m, 1H), 6.11 (s, 1H), 6.08 (d, J=6.10 Hz, 1H), 5.38 (tt, J=3.59, 7.10 Hz, 1H), 3.88 (s, 3H), 3.74 (dd, J=3.36, 11.90 Hz, 1H), 3.48 (br s, 1H), 3.21-3.29 (m, 1H), 3.11 (ddd, J=3.36, 8.09, 11.75 Hz, 1H), 2.05 (ddd, J=3.97, 8.24, 12.21 Hz, 1H), 1.89-1.98 (m, 1H), 1.80-1.89 (m, 1H), 1.65-1.76 (m, 1H).

### Example 7: (R)-1-(3-((6-(1-methyl-1H-pyrazol-4-yl)pyrazolo[1,5-a]pyrazin-4-yl)oxy)piperidin-1-yl)prop-2-yn-1-one

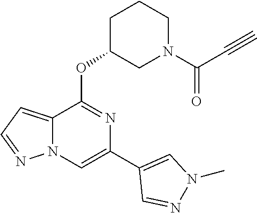 [SMILES: C#CC(=O)N1CCC[C@@H](Oc2nc(-c3cnn(C)c3)cn3nccc23)C1]

### Synthesis of (R)-tert-butyl 3-[6-(1-methylpyrazol-4-yl)pyrazolo[1,5-a]pyrazin-4-yl]oxypiperidine-1-carboxylate

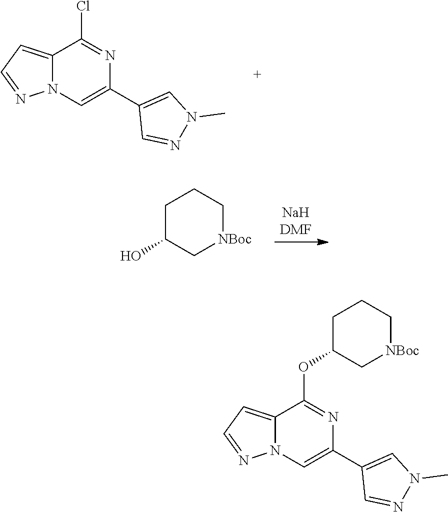 [SMILES: Cn1cc(-c2cn3nccc3c(Cl)n2)cn1 ; CC(C)(C)OC(=O)N1CCC[C@@H](O)C1 ; Cn1cc(-c2cn3nccc3c(O[C@@H]3CCCN(C(=O)OC(C)(C)C)C3)n2)cn1]

A solution of (R)-tert-butyl 3-hydroxypiperidine-1-carboxylate (221 mg, 1.10 mmol) in dry DMF (3 mL) was cooled in an ice bath. Then, sodium hydride (132 mg, 3.30 mmol, 60% purity) was added in 2 batches while stirring. Stirring in the ice bath was continued for 45 minutes during which a pale-yellow suspension formed. To this mixture is added 4-chloro-6-(1-methylpyrazol-4-yl)pyrazolo[1,5-a]pyrazine (234 mg, 1.00 mmol) as one batch and the mixture turned orange-brown instantly. Stirring at RT was continued overnight. The mixture was diluted with EtOAc and then water was added carefully. The mixture was transferred to a separating funnel and the phases were separated. The aq. phase was extracted with EtOAc once more and the combined organic phases were washed with brine, dried with Na₂SO₄, filtered and evaporated in vacuo. The residual material was purified on a 10 g Si-SPE column: Rt=0.22 in heptanes/EtOAc=1/1 to give (R)-tert-butyl 3-[6-(1-methylpyrazol-4-yl)pyrazolo[1,5-a]pyrazin-4-yl]oxypiperidine-1-carboxylate (390 mg, 88% yield, 90% purity) as a colorless viscous gum. LCMS: m/z=399.0 (M+H⁺).

### Synthesis of (R)-6-(1-methylpyrazol-4-yl)-4-(3-piperidyloxy)pyrazolo[1,5-a]pyrazine

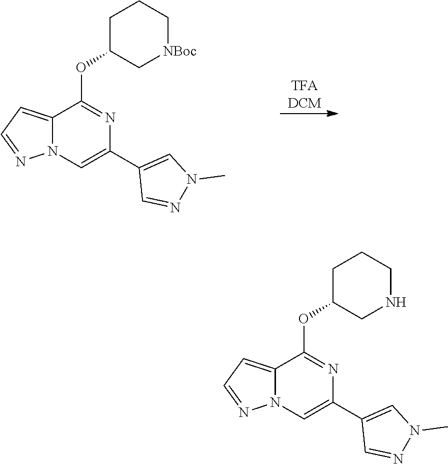 [SMILES: Cn1cc(-c2cn3nccc3c(O[C@@H]3CCCN(C(=O)OC(C)(C)C)C3)n2)cn1 ; Cn1cc(-c2cn3nccc3c(O[C@@H]3CCCNC3)n2)cn1]

To a solution of (R)-tert-butyl 3-[6-(1-methylpyrazol-4-yl)pyrazolo[1,5-a]pyrazin-4-yl]oxypiperidine-1-carboxylate (390 mg, 979 μmol) in dry DCM (3 mL) was added TFA (2.23 g, 19.58 mmol, 1.50 mL) with stirring at RT. After stirring at RT overnight the mixture was diluted with MeOH and purified on a 5 g SCX column where the product eluted with 2 M NH₃-MeOH to give (R)-6-(1-methylpyrazol-4-yl)-4-(3-piperidyloxy)pyrazolo[1,5-a]pyrazine (270 mg, 88% yield, 95% purity) as a colorless gum. LCMS: m/z=299.0 (M+H⁺).

### Synthesis of (R)-1-(3-((6-(1-methyl-1H-pyrazol-4-yl)pyrazolo[1,5-a]pyrazin-4-yl)oxy)piperidin-1-yl)prop-2-yn-1-one

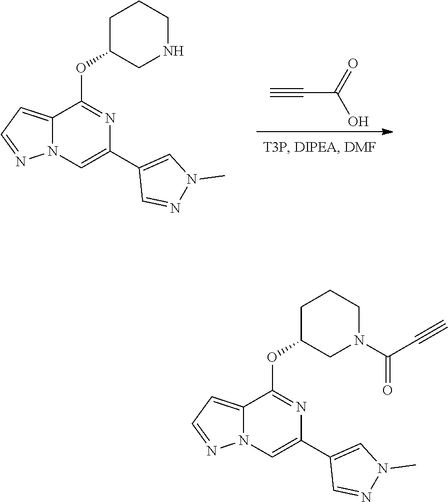

To a solution of (R)-6-(1-methylpyrazol-4-yl)-4-(3-piperidyloxy)pyrazolo[1,5-a]pyrazine (30 mg, 101 μmol) in DMF (1 mL) was added propiolic acid (7.0 mg, 101 μmol, 6 μL) followed by DIPEA (26 mg, 201 μmol, 35 μL) with stirring at RT. Then T3P (128 mg, 201 μmol, 50% purity) was added with stirring. Stirring at RT was continued overnight. The mixture was diluted with EtOAc and washed with water. The organic phase was dried with Na₂SO₄ and filtered. The filtrate was evaporated in vacuo and the residual material was re-dissolved in DMSO. The material was purified with prep HPLC (Waters XSelect CSH C18, 5 μm, 19 mm×100 mm column with mobile phase H₂O (A) and MeCN (B) and a gradient of 5-50% B (0.2% NH₄OH final v/v % modifier) with flow rate at 30 mL/min) to give (R)-1-(3-((6-(1-methyl-1H-pyrazol-4-yl)pyrazolo[1,5-a]pyrazin-4-yl)oxy)piperidin-1-yl)prop-2-yn-1-one (20.6 mg, 95% purity, 55% yield) as a white powder. LCMS: m/z=350.1 (M+H⁺). ¹H NMR (500 MHz, DMSO-d₆) δ 8.75-8.77 (m, 1H), 8.02 (d, J=1.83 Hz, 1H), 8.00-8.27 (m, 2H), 6.76-6.77 (m, 1H), 5.26-5.49 (m, 1H), 4.13-4.61 (m, 1H), 3.90-4.33 (m, 1H), 3.88 (s, 3H), 3.70-3.84 (m, 1H), 3.44-3.62 (m, 1H), 3.15-3.30 (m, 1H), 1.53-2.18 (m, 4H).

### Example 8: (Z)-4-chloro-1-(4-((6-(1-methyl-1H-pyrazol-4-yl)pyrazolo[1,5-a]pyrazin-4-yl)oxy)piperidin-1-yl)but-2-en-1-one

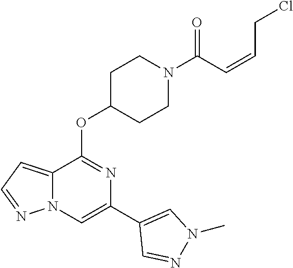 [SMILES: Cn1cc(-c2cn3nccc3c(OC3CCN(C(=O)/C=C\CCl)CC3)n2)cn1]

### Synthesis of (Z)-4-chloro-1-(4-((6-(1-methyl-1H-pyrazol-4-yl)pyrazolo[1,5-a]pyrazin-4-yl)oxy)piperidin-1-yl)but-2-en-1-one

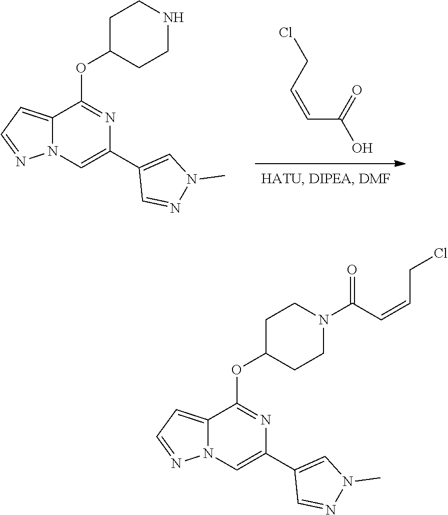 [SMILES: Cn1cc(-c2cn3nccc3c(OC3CCNCC3)n2)cn1 ; O=C(O)/C=C\CCl ; Cn1cc(-c2cn3nccc3c(OC3CCN(C(=O)/C=C\CCl)CC3)n2)cn1]

A 20 mL screw top vial was charged with 6-(1-methylpyrazol-4-yl)-4-(4-piperidyloxy)pyrazolo[1,5-a]pyrazine (30 mg, 100 μmol) and DMF (1 mL). Then (Z)-4-chlorobut-2-enoic acid (15.5 mg, 120 μmol) was added with stirring upon which a milky suspension formed instantaneously. Then, HATU (57.7 mg, 150 μmol) was added and the mixture was stirred at RT for 5 minutes. Then, DIPEA (35 μL, 201 μmol) was added with stirring. Stirring at RT was continued overnight. The mixture was diluted with EtOAc and washed with water. The organic phase was dried with Na₂SO₄ and filtered. The filtrate was evaporated in vacuo and the residual material is dissolved in DMSO and purified with prep HPLC (Waters XSelect CSH C18, 5 μm, 19 mm×100 mm column with mobile phase H₂O (A) and MeCN (B) and a gradient of 5-55% B (0.2% NH₄OH final v/v % modifier) with flow rate at 30 mL/min) to give (Z)-4-chloro-1-(4-((6-(1-methyl-1H-pyrazol-4-yl)pyrazolo[1,5-a]pyrazin-4-yl)oxy)piperidin-1-yl)but-2-en-1-one (17.8 mg, 95% purity, 42% yield) as a white powder. LCMS: m/z=400.1. ¹H NMR (500 MHz, DMSO-d₆) δ 8.76 (s, 1H), 8.21 (s, 1H), 8.00-8.03 (m, 2H), 6.88 (d, J=1.22 Hz, 1H), 6.84-6.87 (m, 1H), 6.65-6.72 (m, 1H), 5.61 (ddd, J=3.66, 7.48, 11.44 Hz, 1H), 4.38 (dd, J=1.22, 6.71 Hz, 2H), 3.89 (s, 3H), 3.44-3.69 (m, 4H), 2.00-2.17 (m, 2H), 1.67-1.86 (m, 2H).

### Example 9: 1-(3-((6-(1-methyl-1H-pyrazol-4-yl)pyrazolo[1,5-a]pyrazin-4-yl)oxy)azetidin-1-yl)prop-2-en-1-one

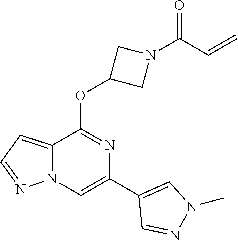 [SMILES: C=CC(=O)N1CC(Oc2nc(-c3cnn(C)c3)cn3nccc23)C1]

### Synthesis of tert-butyl 3-[6-(1-methylpyrazol-4-yl)pyrazolo[1,5-a]pyrazin-4-yl]oxyazetidine-1-carboxylate

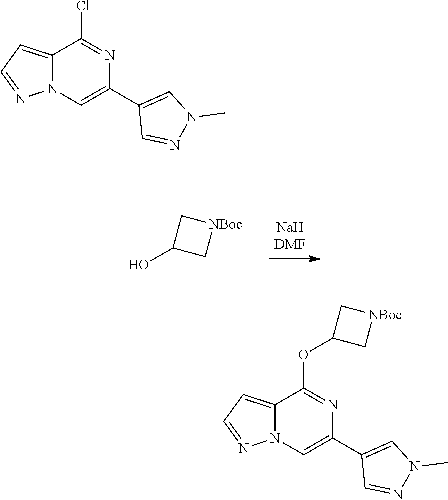 [SMILES: Cn1cc(-c2cn3nccc3c(Cl)n2)cn1 ; CC(C)(C)OC(=O)N1CC(O)C1 ; Cn1cc(-c2cn3nccc3c(OC3CN(C(=O)OC(C)(C)C)C3)n2)cn1]

A solution of tert-butyl 3-hydroxyazetidine-1-carboxylate (191 mg, 1.10 mmol) in dry DMF (3 mL) was cooled in an ice bath. Then, sodium hydride (132 mg, 3.30 mmol, 60% purity) was added in 2 batches while stirring. Stirring in the ice bath was continued for 45 minutes during which a pale-yellow suspension formed. To this mixture was added 4-chloro-6-(1-methylpyrazol-4-yl)pyrazolo[1,5-a]pyrazine (234 mg, 1.00 mmol) as one batch and the mixture turned instantly orange-brown. Stirring at RT was continued overnight. The mixture was diluted with EtOAc and then water is added carefully. The mixture is transferred to a separating funnel and the phases were separated. The aq. phase was extracted with EtOAc once more and the combined organic phases were washed with brine, dried with Na₂SO₄, filtered, and evaporated in vacuo. The residual material was purified on a 10 g Si-SPE column: Rt=0.0.18 in heptanes/EtOAc=1/1 to give tert-butyl 3-[6-(1-methylpyrazol-4-yl)pyrazolo[1,5-a]pyrazin-4-yl]oxyazetidine-1-carboxylate (380 mg, 97% yield, 95% purity) as a colorless viscous gum. ESI-MS (M+H)⁺: 371.0.

### Synthesis of 4-(azetidin-3-yloxy)-6-(1-methylpyrazol-4-yl)pyrazolo[1,5-a]pyrazine

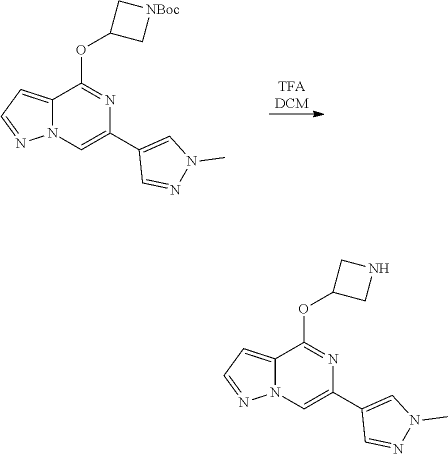 [SMILES: Cn1cc(-c2cn3nccc3c(OC3CN(C(=O)OC(C)(C)C)C3)n2)cn1 ; Cn1cc(-c2cn3nccc3c(OC3CNC3)n2)cn1]

To a solution of tert-butyl 3-[6-(1-methylpyrazol-4-yl)pyrazolo[1,5-a]pyrazin-4-yl]oxyazetidine-1-carboxylate (380 mg, 1.03 mmol) in DCM (5 mL) was added TFA (2.34 g, 20.5 mmol, 1.57 mL) with stirring at RT. Stirring was continued overnight. The mixture was diluted with MeOH and purified on a 10 g SCX column where the product eluted with 2 M NH₃-MeOH to give 4-(azetidin-3-yloxy)-6-(1-methylpyrazol-4-yl)pyrazolo[1,5-a]pyrazine (250 mg, 85% yield, 95% purity) as a white solid. ESI-MS (M+H)⁺: 271.0.

### Synthesis of 1-(3-((6-(1-methyl-1H-pyrazol-4-yl)pyrazolo[1,5-a]pyrazin-4-yl)oxy)azetidin-1-yl)prop-2-en-1-one

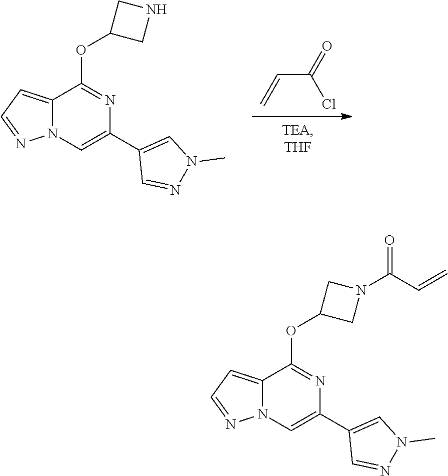

1-(3-((6-(1-methyl-1H-pyrazol-4-yl)pyrazolo[1,5-a]pyrazin-4-yl)oxy)azetidin-1-yl)prop-2-en-1-one was prepared in the same way as Example 2. The material was purified with prep HPLC (Waters XSelect CSH C18, 5 μm, 19 mm×100 mm column with mobile phase H₂O (A) and MeCN (B) and a gradient of 5-45% B (0.2% NH₄OH final v/v % modifier) with flow rate at 30 mL/min) to give 1-(3-((6-(1-methyl-1H-pyrazol-4-yl)pyrazolo[1,5-a]pyrazin-4-yl)oxy)azetidin-1-yl)prop-2-en-1-one (20 mg, 95% purity, 64% yield) as a white powder. LCMS: m/z=325.0. ¹H NMR (500 MHz, DMSO-d₆) δ 8.82 (s, 1H), 8.24 (s, 1H), 8.06 (d, J=2.44 Hz, 1H), 8.03 (s, 1H), 6.90-6.92 (m, 1H), 6.38 (dd, J=10.38, 17.09 Hz, 1H), 6.11-6.17 (m, 1H), 5.70 (dd, J=2.44, 10.38 Hz, 1H), 5.62 (tt, J=4.27, 6.71 Hz, 1H), 4.83 (br dd, J=6.71, 9.16 Hz, 1H), 4.52 (br dd, J=7.02, 11.29 Hz, 1H), 4.37 (br dd, J=3.66, 9.77 Hz, 1H), 4.08 (br dd, J=3.66, 11.60 Hz, 1H), 3.89 (s, 3H).

### Example 10: (Z)-6-(1-methyl-1H-pyrazol-4-yl)-4-((1-(prop-1-en-1-ylsulfonyl)azetidin-3-yl)oxy)pyrazolo[1,5-a]pyrazine

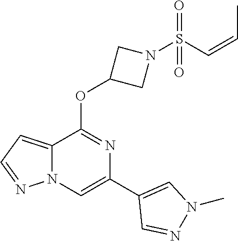 [SMILES: C/C=C\S(=O)(=O)N1CC(Oc2nc(-c3cnn(C)c3)cn3nccc23)C1]

(Z)-6-(1-methyl-1H-pyrazol-4-yl)-4-((1-(prop-1-en-1-ylsulfonyl)azetidin-3-yl)oxy)pyrazolo[1,5-a]pyrazine was prepared in a way similar to Example 9 except with (Z)-prop-1-ene-1-sulfonyl chloride instead of acryloyl chloride. The material was purified with prep HPLC (Waters XSelect CSH C18, 5 μm, 19 mm×100 mm column with mobile phase H₂O (A) and MeCN (B) and a gradient of 5-55% B (0.2% NH₄OH final v/v % modifier) with flow rate at 30 mL/min) to give (Z)-6-(1-methyl-1H-pyrazol-4-yl)-4-((1-(prop-1-en-1-ylsulfonyl)azetidin-3-yl)oxy)pyrazolo[1,5-a]pyrazine (10 mg, 95% purity, 28% yield) as a beige solid. LCMS: m/z=374.0. ¹H NMR (500 MHz, DMSO-d₆) δ 8.82 (s, 1H), 8.23-8.27 (s, 1H), 8.06 (d, J=2.44 Hz, 1H), 7.98-8.04 (s, 1H), 6.87-6.92 (m, 1H), 6.72-6.84 (m, 2H), 5.42-5.56 (m, 1H), 4.31-4.45 (m, 2H), 3.92-4.04 (m, 2H), 3.88 (s, 3H), 1.95 (d, J=4.88 Hz, 3H).

### Example 11: (R)-1-(3-((6-(1-methyl-1H-pyrazol-4-yl)pyrazolo[1,5-a]pyrazin-4-yl)oxy)pyrrolidin-1-yl)prop-2-en-1-one

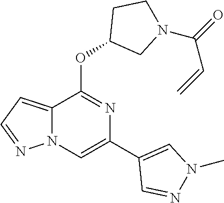 [SMILES: C=CC(=O)N1CC[C@@H](Oc2nc(-c3cnn(C)c3)cn3nccc23)C1]

### Synthesis of tert-butyl (3R)-3-[6-(1-methylpyrazol-4-yl)pyrazolo[1,5-a]pyrazin-4-yl]oxypyrrolidine-1-carboxylate

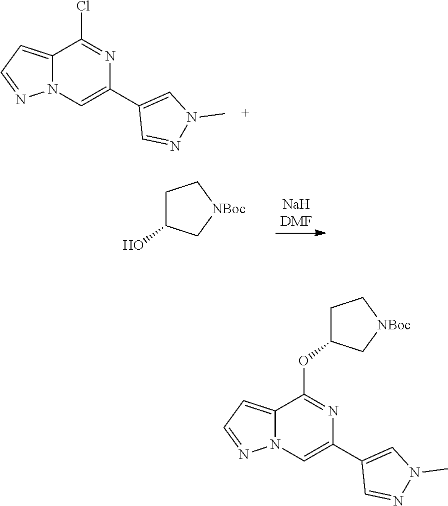

A solution of tert-butyl (3R)-3-hydroxypyrrolidine-1-carboxylate (193 mg, 1.03 mmol) in dry DMF (3 mL) was cooled in an ice bath. Then, sodium hydride (136 mg, 3.40 mmol, 60% purity) was added in 4 batches while stirring. Stirring in the ice bath was continued for 45 minutes during which a pale-yellow suspension formed. To this mixture was added 4-chloro-6-(1-methylpyrazol-4-yl)pyrazolo[1,5-a]pyrazine (240 mg, 1.03 mmol) as one batch and the mixture turned orange-brown instantly. Stirring at RT was continued overnight. The mixture was diluted with EtOAc and then water was added carefully. The mixture was transferred to a separating funnel and the phases were separated. The aq. phase was extracted with EtOAc once more and the combined organic phases were washed with brine, dried with Na2SO4, filtered, and evaporated in vacuo. The residual material was purified on a 10 g Si-SPE column in heptanes/EtOAc=1/1 to give tert-butyl (3R)-3-[6-(1-methylpyrazol-4-yl)pyrazolo[1,5-a]pyrazin-4-yl]oxypyrrolidine-1-carboxylate (345 mg, 83% yield, 95% purity) as a sticky colorless gum which turned into a sticky white foam upon further drying. ESI-MS (M+H)+: 395.0.

### Synthesis of 6-(1-methylpyrazol-4-yl)-4-[(3R)-pyrrolidin-3-yl]oxy-pyrazolo[1,5-a]pyrazine

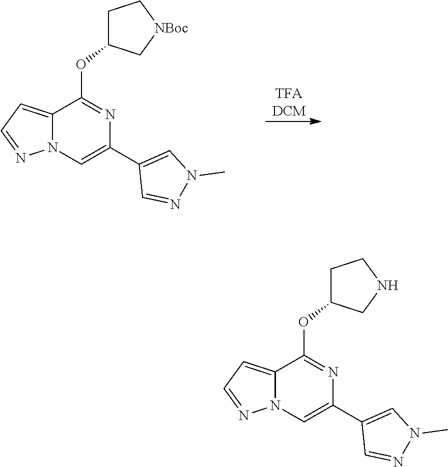 [SMILES: Cn1cc(-c2cn3nccc3c(O[C@@H]3CCN(C(=O)OC(C)(C)C)C3)n2)cn1 ; Cn1cc(-c2cn3nccc3c(O[C@@H]3CCNC3)n2)cn1]

To a solution of tert-butyl (3R)-3-[6-(1-methylpyrazol-4-yl)pyrazolo[1,5-a]pyrazin-4-yl]oxypyrrolidine-1-carboxylate (326 mg, 849 μmol) in DCM (5 mL) was added TFA (1.93 g, 17 mmol, 1.30 mL) with stirring at RT. Stirring was continued overnight. The mixture was diluted with MeOH and purified on a 10 g SCX column where the product eluted with 2 M NH₃-MeOH to give 6-(1-methylpyrazol-4-yl)-4-[(3R)-pyrrolidin-3-yl]oxy-pyrazolo[1,5-a]pyrazine (230 mg, 91% yield, 95% purity) as a sticky pale-yellow gum. ESI-MS (M+H)+: 285.0.

### Synthesis of (R)-1-(3-((6-(1-methyl-1H-pyrazol-4-yl)pyrazolo[1,5-a]pyrazin-4-yl)oxy)pyrrolidin-1-yl)prop-2-en-1-one

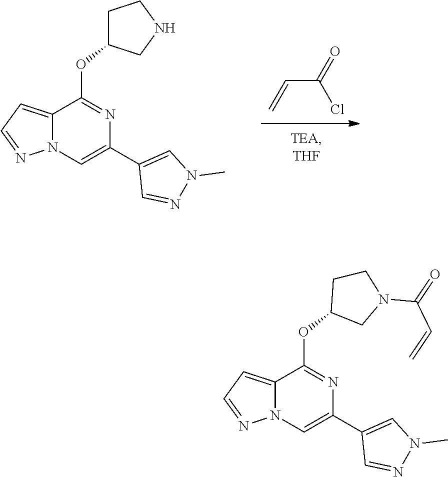

(R)-1-(3-((6-(1-methyl-1H-pyrazol-4-yl)pyrazolo[1,5-a]pyrazin-4-yl)oxy)pyrrolidin-1-yl)prop-2-en-1-one was prepared in the same way as Example 2. The material was purified with prep HPLC (Waters XSelect CSH C18, 5 μm, 19 mm×100 mm column with mobile phase H₂O (A) and MeCN (B) and a gradient of 5-45% B (0.2% NH₄OH final v/v % modifier) with flow rate at 30 mL/min) to give (R)-1-(3-((6-(1-methyl-1H-pyrazol-4-yl)pyrazolo[1,5-a]pyrazin-4-yl)oxy)pyrrolidin-1-yl)prop-2-en-1-one (27.9 mg, 95% purity, 74% yield) as a white solid. ESI-MS (M+H)+: 339.0. ¹H NMR (500 MHz, DMSO-d₆) δ 8.78 (s, 1H), 8.21-8.26 (m, 1H), 8.03 (d, J=1.83 Hz, 1H), 8.00-8.02 (m, 1H), 6.84 (br d, J=1.22 Hz, 1H), 6.54-6.69 (m, 1H), 6.12-6.20 (m, 1H), 5.81-5.94 (m, 1H), 5.64-5.73 (m, 1H), 3.89 (s, 3H), 3.82-4.11 (m, 1H), 3.65-3.78 (m, 1H), 3.43-3.59 (m, 2H), 2.19-2.47 (m, 2H).

### Example 12: (R)-6-(1-methyl-1H-pyrazol-4-yl)-4-((1-(vinylsulfonyl)pyrrolidin-3-yl)oxy)pyrazolo[1,5-a]pyrazine

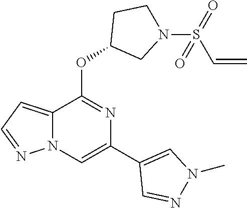 [SMILES: C=CS(=O)(=O)N1CC[C@@H](Oc2nc(-c3cnn(C)c3)cn3nccc23)C1]

(R)-6-(1-methyl-1H-pyrazol-4-yl)-4-((1-(vinylsulfonyl)pyrrolidin-3-yl)oxy)pyrazolo[1,5-a]pyrazine was prepared in a way similar to Example 11 except replaced acryloyl chloride with 2-Chloro-ethane-sulfonyl chloride. The material was purified with prep HPLC (Waters XSelect CSH C18, 5 μm, 19 mm×100 mm column with mobile phase H₂O (A) and MeCN (B) and a gradient of 5-50% B (0.2% NH₄OH final v/v % modifier) with flow rate at 30 mL/min) to give (R)-6-(1-methyl-1H-pyrazol-4-yl)-4-((1-(vinylsulfonyl)pyrrolidin-3-yl)oxy)pyrazolo[1,5-a]pyrazine (6.6 mg, 95% purity, 16% yield) as a white solid. ESI-MS (M+H)⁺: 375.0. ¹H NMR (500 MHz, DMSO-d₆) δ 8.78 (s, 1H), 8.22 (s, 1H), 8.03 (d, J=2.44 Hz, 1H), 8.00-8.02 (m, 1H), 6.92 (dd, J=10.38, 16.48 Hz, 1H), 6.85-6.86 (m, 1H), 6.07-6.12 (m, 1H), 6.05 (d, J=9.77 Hz, 1H), 5.78-5.84 (m, 1H), 3.88 (s, 3H), 3.39-3.75 (m, 4H), 2.20-2.39 (m, 2H).

### Example 13: (R)-1-(3-((6-(1-methyl-1H-pyrazol-4-yl)pyrazolo[1,5-a]pyrazin-4-yl)oxy)pyrrolidin-1-yl)prop-2-yn-1-one

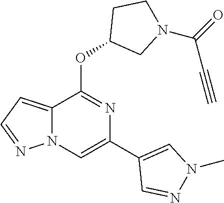 [SMILES: C#CC(=O)N1CC[C@@H](Oc2nc(-c3cnn(C)c3)cn3nccc23)C1]

(R)-1-(3-((6-(1-methyl-1H-pyrazol-4-yl)pyrazolo[1,5-a]pyrazin-4-yl)oxy)pyrrolidin-1-yl)prop-2-yn-1-one was prepared in a way similar to Example 1. The material was purified with prep HPLC (Waters XSelect CSH C18, 5 μm, 19 mm×100 mm column with mobile phase H₂O (A) and MeCN (B) and a gradient of 5-45% B (0.2% NH₄OH final v/v % modifier) with flow rate at 30 mL/min) to give (R)-1-(3-((6-(1-methyl-1H-pyrazol-4-yl)pyrazolo[1,5-a]pyrazin-4-yl)oxy)pyrrolidin-1-yl)prop-2-yn-1-one (18.2 mg, 95% purity, 51% yield) as a white solid. ESI-MS (M+H)⁺: 337.0. ¹H NMR (500 MHz, DMSO-d₆) δ 8.77-8.81 (m, 1H), 8.21-8.26 (m, 1H), 8.00-8.06 (m, 2H), 6.86 (d, J=2.44 Hz, 1H), 5.81-5.91 (m, 1H), 4.43-4.58 (m, 1H), 3.86-3.91 (m, 1H), 3.86 (s, 3H), 3.40-3.71 (m, 3H), 2.20-2.47 (m, 2H).

### Example 14: (S)-1-(3-(((6-(1-methyl-1H-pyrazol-4-yl)pyrazolo[1,5-a]pyrazin-4-yl)oxy)methyl)piperidin-1-yl)prop-2-en-1-one

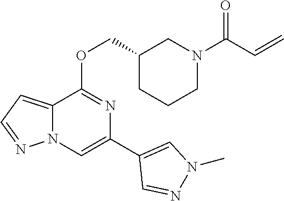 [SMILES: C=CC(=O)N1CCC[C@H](COc2nc(-c3cnn(C)c3)cn3nccc23)C1]

### Synthesis of tert-butyl (3S)-3-[[6-(1-methylpyrazol-4-yl)pyrazolo[1,5-a]pyrazin-4-yl]oxymethyl]piperidine-1-carboxylate

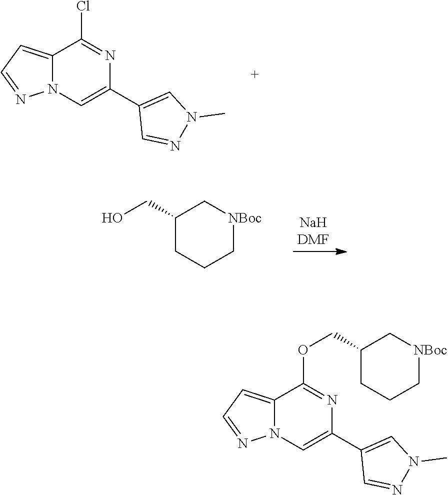 [SMILES: Cn1cc(-c2cn3nccc3c(Cl)n2)cn1 ; CC(C)(C)OC(=O)N1CCC[C@H](CO)C1 ; Cn1cc(-c2cn3nccc3c(OC[C@H]3CCCN(C(=O)OC(C)(C)C)C3)n2)cn1]

A solution of tert-butyl (3S)-3-(hydroxymethyl)piperidine-1-carboxylate (222 mg, 1.03 mmol) in dry DMF (3 mL) was cooled in an ice bath. Then, sodium hydride (136 mg, 3.40 mmol, 60% purity) was added in 4 batches while stirring. Stirring in the ice bath was continued for 45 minutes during which a pale-yellow suspension formed. To this mixture was added 4-chloro-6-(1-methylpyrazol-4-yl)pyrazolo[1,5-a]pyrazine (240 mg, 1.03 mmol) as one batch and the mixture turned orange-brown instantly. Stirring at RT was continued overnight. The mixture was diluted with EtOAc and then water was added carefully. The mixture was transferred to a separating funnel and the phases were separated. The aq. phase was extracted with EtOAc once more and the combined organic phases were washed with brine, dried with Na₂SO₄, filtered, and evaporated in vacuo. The residual material was purified on a 10 g Si-SPE column: Rt=0.1 in heptanes/EtOAc=2/1 to give tert-butyl (3S)-3-[[6-(1-methylpyrazol-4-yl)pyrazolo[1,5-a]pyrazin-4-yl]oxymethyl]piperidine-1-carboxylate (386 mg, 86% yield, 95% purity) as an off-white solid; ESI-MS (M+H)+: 413.0.

### Synthesis of 6-(1-methylpyrazol-4-yl)-4-[[(3S)-3-piperidyl]methoxy]pyrazolo[1,5-a]pyrazine

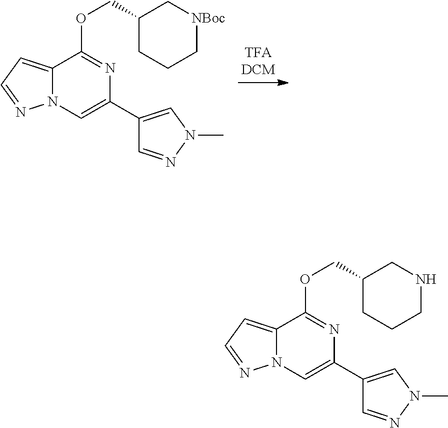 [SMILES: Cn1cc(-c2cn3nccc3c(OC[C@H]3CCCN(C(=O)OC(C)(C)C)C3)n2)cn1 ; Cn1cc(-c2cn3nccc3c(OC[C@H]3CCCNC3)n2)cn1]

To a solution of tert-butyl (3S)-3-[[6-(1-methylpyrazol-4-yl)pyrazolo[1,5-a]pyrazin-4-yl]oxymethyl]piperidine-1-carboxylate (404 mg, 978 μmol) in DCM (5 mL) was added TFA (2.23 g, 19.6 mmol, 1.50 mL) with stirring at RT. Stirring was continued overnight. The mixture was diluted with MeOH and purified on a 10 g SCX column where the product eluted with 2 M NH₃-MeOH to give 6-(1-methylpyrazol-4-yl)-4-[[(3S)-3-piperidyl]methoxy]pyrazolo[1,5-a]pyrazine (240 mg, 75% yield, 95% purity) as a pale-yellow sticky gum which formed a white foam upon further drying. ESI-MS (M+H)+: 313.0.

### Synthesis of (S)-1-(3-(((6-(1-methyl-1H-pyrazol-4-yl)pyrazolo[1,5-a]pyrazin-4-yl)oxy)methyl)piperidin-1-yl)prop-2-en-1-one

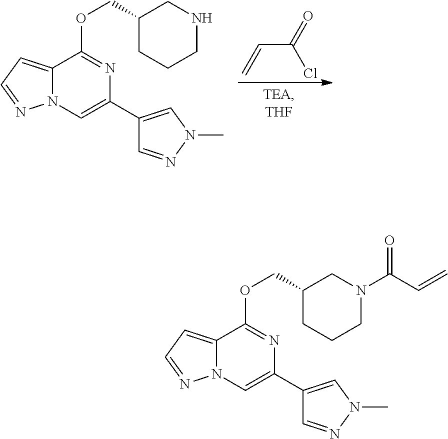

(S)-1-(3-(((6-(1-methyl-1H-pyrazol-4-yl)pyrazolo[1,5-a]pyrazin-4-yl)oxy)methyl)piperidin-1-yl)prop-2-en-1-one was prepared in the same way as Example 2. The material was purified with prep HPLC (Waters XSelect CSH C18, 5 μm, 19 mm×100 mm column with mobile phase H₂O (A) and MeCN (B) and a gradient of 5-50% B (0.2% NH₄OH final v/v % modifier) with flow rate at 30 mL/min) to give (S)-1-(3-(((6-(1-methyl-1H-pyrazol-4-yl)pyrazolo[1,5-a]pyrazin-4-yl)oxy)methyl)piperidin-1-yl)prop-2-en-1-one (10.6 mg, 95% purity, 28% yield) as a white powder. ESI-MS (M+H)+: 367.0. ¹H NMR (500 MHz, DMSO-d₆) δ 8.76 (s, 1H), 8.21 (br s, 1H), 8.03 (d, J=2.44 Hz, 1H), 8.01 (s, 1H), 6.85-6.92 (m, 1H), 6.77-6.84 (m, 1H), 5.96-6.10 (m, 1H), 5.54-5.67 (m, 1H), 4.41-4.50 (m, 2H), 3.90-4.13 (m, 1H), 3.89 (s, 3H), 2.89-3.29 (m, 3H), 1.88-2.07 (m, 2H), 1.73 (s, 1H), 1.35-1.57 (m, 2H).

### Example 15: (S)-6-(1-methyl-1H-pyrazol-4-yl)-4-((1-(vinylsulfonyl)piperidin-3-yl)methoxy)pyrazolo[1,5-a]pyrazine

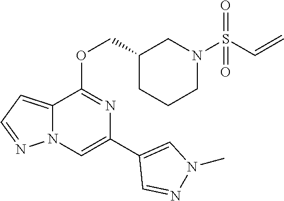 [SMILES: C=CS(=O)(=O)N1CCC[C@H](COc2nc(-c3cnn(C)c3)cn3nccc23)C1]

(S)-6-(1-methyl-1H-pyrazol-4-yl)-4-((1-(vinylsulfonyl)piperidin-3-yl)methoxy)pyrazolo[1,5-a]pyrazine was prepared in a way similar to Example 14 except replaced acryloyl chloride with 2-chloro-ethane-sulfonyl chloride. The material was purified with prep HPLC (Waters SunFire Prep C18, 5 μm, OBD 19 mm×100 mm column with mobile phase H₂O (A) and MeCN (B) and a gradient of 5-55% B (0.1% TFA final v/v % modifier) with flow rate at 30 m/min) to give (S)-6-(1-methyl-1H-pyrazol-4-yl)-4-((1-(vinylsulfonyl)piperidin-3-yl)methoxy)pyrazolo[1,5-a]pyrazine (8.0 mg, 95% purity, 19% yield) as a beige solid. ESI-MS (M+H)+: 403.0. ¹H NMR (500 MHz, DMSO-d₆) δ 8.77 (s, 1H), 8.21 (s, 1H), 8.03 (d, J=2.44 Hz, 1H), 8.01 (s, 1H), 6.84 (d, J=3.05 Hz, 1H), 6.76-6.84 (m, 1H), 6.13 (d, J=9.77 Hz, 1H), 6.09 (d, J=16.48 Hz, 1H), 4.42-4.52 (m, 2H), 3.89 (s, 3H), 3.63 (br dd, J=3.66, 11.60 Hz, 1H), 3.38-3.55 (m, 2H), 2.59-2.74 (m, 1H), 2.12-2.28 (m, 1H), 1.84-1.95 (m, 1H), 1.79 (td, J=3.66, 13.43 Hz, 1H), 1.50-1.64 (m, 1H), 1.18-1.38 (m, 1H).

### Example 16: (S)-1-(3-(((6-(1-methyl-1H-pyrazol-4-yl)pyrazolo[1,5-a]pyrazin-4-yl)oxy)methyl)piperidin-1-yl)prop-2-yn-1-one

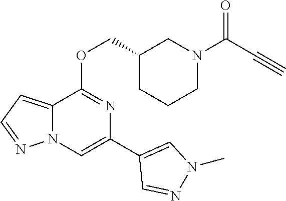 [SMILES: C#CC(=O)N1CCC[C@H](COc2nc(-c3cnn(C)c3)cn3nccc23)C1]

(S)-1-(3-(((6-(1-methyl-1H-pyrazol-4-yl)pyrazolo[1,5-a]pyrazin-4-yl)oxy)methyl)piperidin-1-yl)prop-2-yn-1-one was prepared in a way similar to Example 1. The material was purified with prep HPLC (Waters XSelect CSH C18, 5 μm, 19 mm×100 mm column with mobile phase H₂O (A) and MeCN (B) and a gradient of 5-55% B (0.2% NH₄OH final v/v % modifier) with flow rate at 30 mL/min) to give (S)-1-(3-(((6-(1-methyl-1H-pyrazol-4-yl)pyrazolo[1,5-a]pyrazin-4-yl)oxy)methyl)piperidin-1-yl)prop-2-yn-1-one (24.8 mg, 95% purity, 68% yield) as a white solid. ESI-MS (M+H)+: 365.0. ¹H NMR (500 MHz, DMSO-d₆) δ 8.74-8.78 (m, 1H), 8.21 (s, 1H), 7.96-8.07 (m, 2H), 6.78-6.90 (m, 1H), 4.52-4.55 (m, 1H), 4.27-4.51 (m, 2H), 4.01-4.17 (m, 1H), 3.89 (s, 3H), 3.22-3.32 (m, 1H), 2.78-2.98 (m, 2H), 1.98-2.21 (m, 1H), 1.88-1.95 (m, 1H), 1.67-1.83 (m, 1H), 1.28-1.55 (m, 2H).

### Example 17: (R,E)-6-(1-methyl-1H-pyrazol-4-yl)-4-((1-(prop-1-en-1-ylsulfonyl)piperidin-3-yl)oxy)pyrazolo[1,5-a]pyrazine

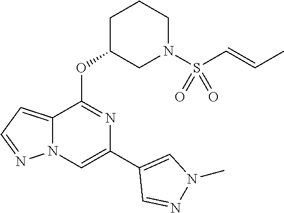 [SMILES: C/C=C/S(=O)(=O)N1CCC[C@@H](Oc2nc(-c3cnn(C)c3)cn3nccc23)C1]

### 1. Synthesis of (R,E)-6-(1-methyl-1H-pyrazol-4-yl)-4-((1-(prop-1-en-1-ylsulfonyl)piperidin-3-yl)oxy)pyrazolo[1,5-a]pyrazine

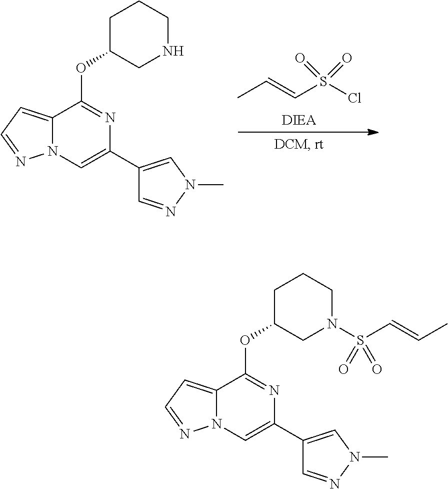

To a vial was added 6-(1-methylpyrazol-4-yl)-4-[[(3R)-3-piperidyl]oxy]pyrazolo[1,5-a]pyrazine (125 mg, 419 μmol), DCM (2.1 mL), N-ethyl-N-isopropyl-propan-2-amine (162 mg, 1.26 mmol, 220 μL) and (E)-prop-1-ene-1-sulfonyl chloride (88 mg, 628 μmol, 66 μL) in that order. The vial was stirred at room temperature overnight. The reaction was diluted with water, passed through a phase separator, and concentrated. The material was dissolved in 2.5 mL of DMSO and passed through a syringe filter. The material was purified via reverse phase HPLC (Waters XSelect CSH C18, 5 μm, 19 mm×100 mm column with mobile phase H₂O (A) and MeCN (B) and a gradient of 5-60% B (0.2% NH₄OH final v/v % modifier) with flow rate at 30 mL/min) to afford 6-(1-methylpyrazol-4-yl)-4-[[(3R)-1-[(E)-prop-1-enyl]sulfonyl-3-piperidyl]oxy]pyrazolo[1,5-a]pyrazine (55 mg, yield: 30%) as an off-white solid. ESI-MS (M+H)⁺: 403.1. ¹H NMR (500 MHz, DMSO-d₆) δ 8.78 (s, 1H), 8.21 (s, 1H), 7.99-8.06 (m, 1H), 6.84 (dd, J=1.22, 2.44 Hz, 1H), 6.61 (br d, J=6.71 Hz, 1H), 6.46-6.54 (m, 1H), 5.38 (td, J=3.89, 7.48 Hz, 1H), 3.88 (s, 3H), 3.72 (br dd, J=3.36, 11.90 Hz, 1H), 3.12-3.28 (m, 2H), 3.05 (ddd, J=3.36, 8.09, 11.75 Hz, 1H), 2.02-2.08 (m, 1H), 1.90-1.98 (m, 2H), 1.85 (dd, J=1.83, 6.71 Hz, 3H), 1.71 (ddd, J=4.58, 8.55, 13.12 Hz, 2H).

### Example 18: 1-(4-(((6-(1-methyl-1H-pyrazol-4-yl)pyrazolo[1,5-a]pyrazin-4-yl)amino)methyl)piperidin-1-yl)prop-2-en-1-one

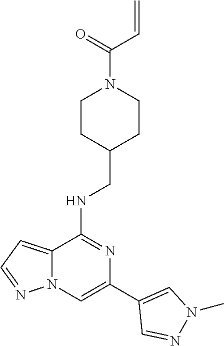 [SMILES: C=CC(=O)N1CCC(CNc2nc(-c3cnn(C)c3)cn3nccc23)CC1]

### 1. Synthesis of tert-butyl 4-(((6-(1-methyl-1H-pyrazol-4-yl)pyrazolo[1,5-a]pyrazin-4-yl)amino)methyl)piperidine-1-carboxylate

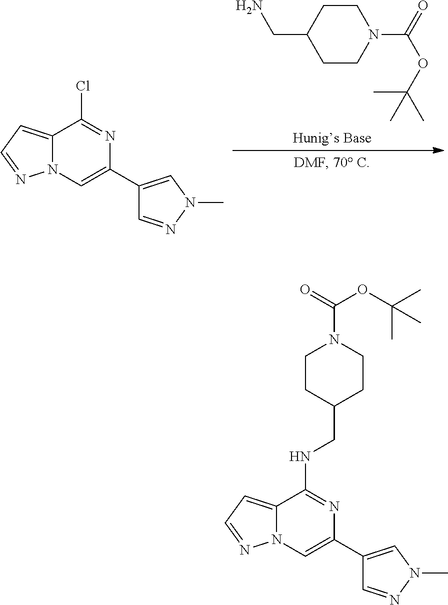 [SMILES: Cn1cc(-c2cn3nccc3c(Cl)n2)cn1 ; CC(C)(C)OC(=O)N1CCC(CN)CC1 ; Cn1cc(-c2cn3nccc3c(NCC3CCN(C(=O)OC(C)(C)C)CC3)n2)cn1]

To a suspension of 4-chloro-6-(1-methylpyrazol-4-yl)pyrazolo[1,5-a]pyrazine (500 mg, 2.14 mmol) and tert-butyl 4-(aminomethyl)piperidine-1-carboxylate (504 mg, 2.35 mmol, 500 μL) in DMF (7.13 mL) was added Hunig's base (553 mg, 4.28 mmol, 750 μL). The reaction was warmed to 70° C. and stirred overnight. The reaction was concentrated and purified via column chromatography (40 g silica column, gradient elution 0-100% EtOAc: heptanes) to afford tert-butyl 4-[[[6-(1-methylpyrazol-4-yl)pyrazolo[1,5-a]pyrazin-4-yl]amino]methyl]piperidine-1-carboxylate (398 mg, yield: 45%) as a brown solid. ESI-MS (M+H)⁺: 412.2.

### 2. Synthesis of 6-(1-methyl-1H-pyrazol-4-yl)-N-(piperidin-4-ylmethyl)pyrazolo[1,5-a]pyrazin-4-amine

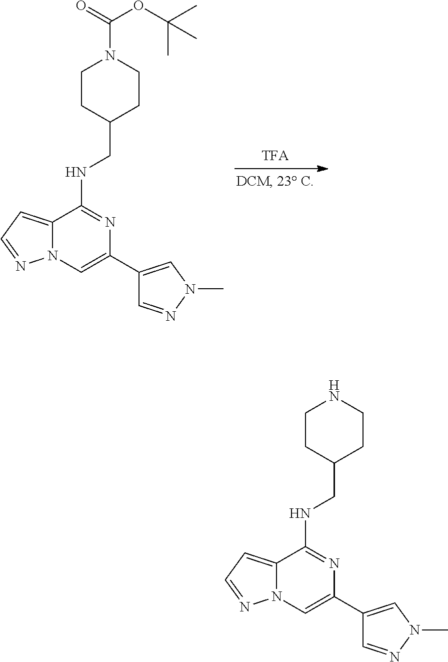 [SMILES: Cn1cc(-c2cn3nccc3c(NCC3CCN(C(=O)OC(C)(C)C)CC3)n2)cn1 ; Cn1cc(-c2cn3nccc3c(NCC3CCNCC3)n2)cn1]

tert-Butyl 4-[[[6-(1-methylpyrazol-4-yl)pyrazolo[1,5-a]pyrazin-4-yl]amino]methyl]piperidine-1-carboxylate (397 mg, 965 μmol) was dissolved in DCM (4.8 mL). TFA (1.10 g, 9.65 mmol, 738 μL) was added and the reaction was stirred overnight at room temperature. The reaction was concentrated, diluted with DCM and carefully quenched with saturated sodium bicarbonate solution. The aqueous layer was washed twice with DCM, then diluted with saturated ammonium hydroxide solution and extracted twice with ethyl acetate. The combined ethyl acetate layers were washed with brine, dried over magnesium sulfate, filtered, and concentrated to afford 6-(1-methylpyrazol-4-yl)-N-(4-piperidylmethyl)pyrazolo[1,5-a]pyrazin-4-amine (300 mg, yield: 100%) as a light yellow oily solid. ESI-MS (M+H)⁺: 312.1.

### 3. Synthesis of 1-(4-(((6-(1-methyl-1H-pyrazol-4-yl)pyrazolo[1,5-a]pyrazin-4-yl)amino)methyl)piperidin-1-yl)prop-2-en-1-one

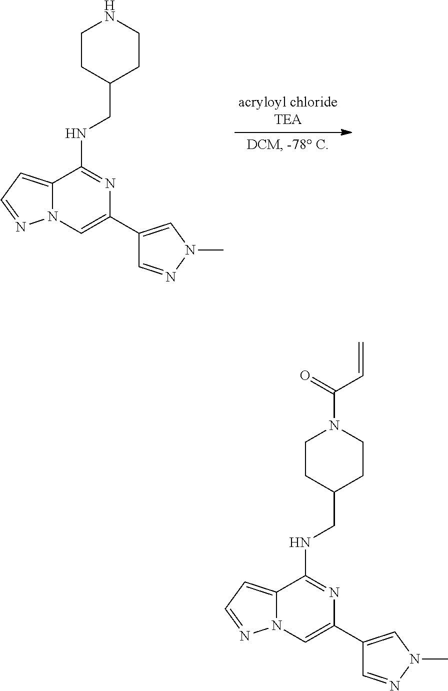 [SMILES: Cn1cc(-c2cn3nccc3c(NCC3CCNCC3)n2)cn1 ; C=CC(=O)N1CCC(CNc2nc(-c3cnn(C)c3)cn3nccc23)CC1]

To a vial containing 6-(1-methylpyrazol-4-yl)-N-(4-piperidylmethyl)pyrazolo[1,5-a]pyrazin-4-amine (75 mg, 241 μmol) was added DCM (2.4 mL) and TEA (73 mg, 725 μmol, 100 μL) which was then placed on a dry ice/acetone bath for 10 minutes. To this solution was added acryloyl chloride (28 mg, 313 μmol, 26 μL) dropwise. The reaction was stirred for 10 minutes. The reaction was diluted with water and passed through a phase separator. The aqueous layer was extracted with DCM and the combined organic layers were concentrated, dissolved in DMSO, and purified via reverse phase HPLC (Waters XSelect CSH C18, 5 μm, 19 mm×100 mm column with mobile phase H₂O (A) and MeCN (B) and a gradient of 5-35% B (0.2% NH₄OH final v/v % modifier) with flow rate at 30 mL/min.) to afford 1-[4-[[[6-(1-methylpyrazol-4-yl)pyrazolo[1,5-a]pyrazin-4-yl]amino]methyl]-1-piperidyl]prop-2-en-1-one (35 mg, yield: 39%) as a yellow solid. ESI-MS (M+H)⁺: 366.2. ¹H NMR (500 MHz, DMSO-d₆) δ 8.27 (s, 1H), 8.09 (s, 1H), 7.92 (s, 1H), 7.84 (d, J=2.44 Hz, 1H), 7.64 (br t, J=5.49 Hz, 1H), 6.93-6.97 (m, 1H), 6.80 (br dd, J=10.68, 16.79 Hz, 1H), 6.07 (br dd, J=2.44, 17.09 Hz, 1H), 5.61-5.68 (m, 1H), 4.42 (br d, J=12.21 Hz, 1H), 4.06 (br d, J=12.82 Hz, 1H), 3.87 (s, 3H), 3.40-3.50 (m, 1H), 2.97-3.10 (m, 1H), 2.59-2.67 (m, 1H), 1.99 (ddd, J=3.97, 7.17, 10.83 Hz, 2H), 1.81 (br d, J=13.43 Hz, 2H), 1.06-1.22 (m, 2H).

### Example 19: 6-(1-methyl-1H-pyrazol-4-yl)-N-((1-(vinylsulfonyl)piperidin-4-yl)methyl)pyrazolo[1,5-a]pyrazin-4-amine

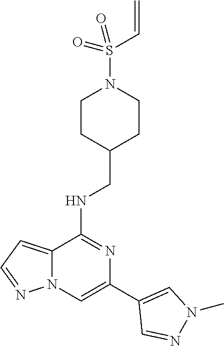 [SMILES: C=CS(=O)(=O)N1CCC(CNc2nc(-c3cnn(C)c3)cn3nccc23)CC1]

### 1. Synthesis of 6-(1-methyl-1H-pyrazol-4-yl)-N-((1-(vinylsulfonyl)piperidin-4-yl)methyl)pyrazolo[1,5-a]pyrazin-4-amine

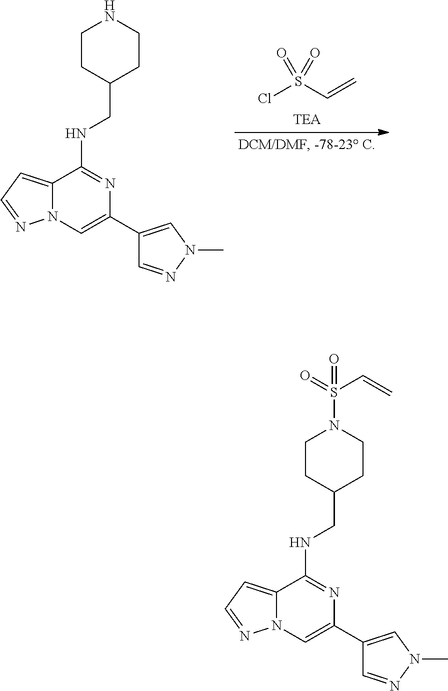

To a vial containing 6-(1-methylpyrazol-4-yl)-N-(4-piperidylmethyl)pyrazolo[1,5-a]pyrazin-4-amine (75 mg, 241 μmol) was added DCM (2.4 mL), DMF (200 μL) and TEA (73 mg, 725 μmol, 100 μL) which was then placed in a dry ice/acetone bath for 10 minutes. To this solution was added ethenesulfonyl chloride (40 mg, 313 μmol, 28 μL) dropwise. The reaction was warmed to room temperature and stirred overnight. The reaction was diluted with water and passed through a phase separator. The aqueous layer was extracted with DCM and the combined organic layers were concentrated, dissolved in DMSO, and purified via reverse phase HPLC (Waters XSelect CSH C18, 5 μm, 19 mm×100 mm column with mobile phase H₂O (A) and MeCN (B) and a gradient of 5-40% B (0.2% NH₄OH final v/v % modifier) with flow rate at 30 mL/min.) to afford 6-(1-methylpyrazol-4-yl)-N-[(1-vinylsulfonyl-4-piperidyl)methyl]pyrazolo-[1,5-a]pyrazin-4-amine (19 mg, yield: 20%) as a yellow solid. ESI-MS (M+H)⁺: 402.2. ¹H NMR (500 MHz, DMSO-d₆) δ 8.27 (s, 1H), 8.09 (s, 1H), 7.92 (s, 1H), 7.82-7.86 (m, 1H), 7.66 (br t, J=5.80 Hz, 1H), 6.93-6.96 (m, 1H), 6.78 (dd, J=9.77, 16.48 Hz, 1H), 6.06-6.15 (m, 2H), 3.87 (s, 3H), 3.55 (br d, J=11.60 Hz, 2H), 2.57-2.65 (m, 4H), 1.85 (br d, J=11.60 Hz, 3H), 1.25-1.35 (m, 2H).

### Example 20: N-[1-[6-(1-methylpyrazol-4-yl)pyrazolo[1,5-a]pyrazin-4-yl]-3-piperidyl]prop-2-enamide

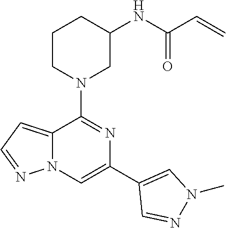 [SMILES: C=CC(=O)NC1CCCN(c2nc(-c3cnn(C)c3)cn3nccc23)C1]

### Synthesis of tert-butyl N-[1-[6-(1-methylpyrazol-4-yl)pyrazolo[1,5-a]pyrazin-4-yl]-3-piperidyl]carbamate

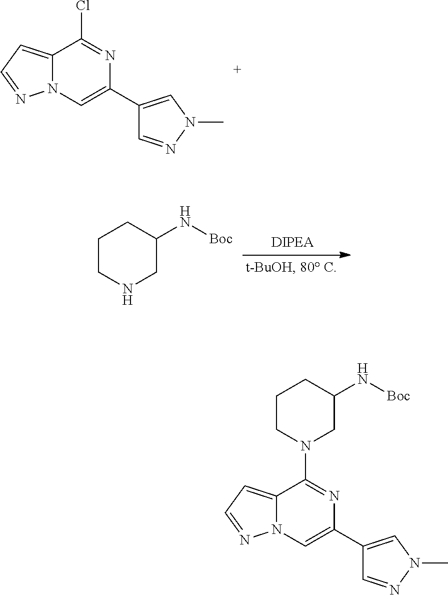 [SMILES: Cn1cc(-c2cn3nccc3c(Cl)n2)cn1 ; CC(C)(C)OC(=O)NC1CCCNC1 ; Cn1cc(-c2cn3nccc3c(N3CCCC(NC(=O)OC(C)(C)C)C3)n2)cn1]

To a solution of 4-chloro-6-(1-methylpyrazol-4-yl)pyrazolo[1,5-a]pyrazine (300 mg, 1.28 mmol) in t-BuOH (5.1 mL) was added N-ethyl-N-isopropyl-propan-2-amine (249 mg, 1.93 mmol, 336 μL) and tert-butyl N-(3-piperidyl)carbamate (264 mg, 1.32 mmol) in that order. The reaction mixture was stirred at 80° C. overnight. The material was concentrated and taken forward as crude assuming 100% yield. LCMS m/z=398.0. (M+H)+.

### Synthesis of 1-[6-(1-methylpyrazol-4-yl)pyrazolo[1,5-a]pyrazin-4-yl]piperidin-3-amine hydrochloride

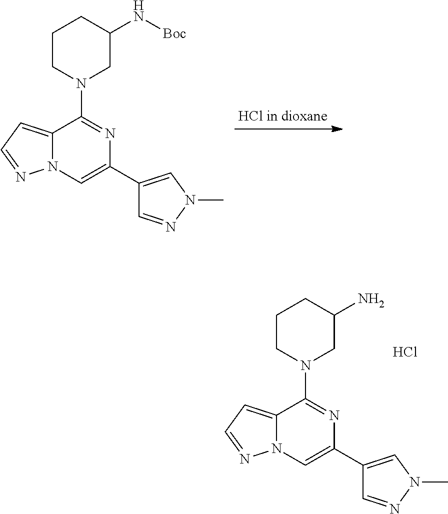 [SMILES: Cn1cc(-c2cn3nccc3c(N3CCCC(NC(=O)OC(C)(C)C)C3)n2)cn1 ; Cn1cc(-c2cn3nccc3c(N3CCCC(N)C3)n2)cn1 ; Cl]

To a solution of tert-butyl N-[1-[6-(1-methylpyrazol-4-yl)pyrazolo[1,5-a]pyrazin-4-yl]-3-piperidyl]carbamate (509 mg, 1.28 mmol) in dioxane (6.4 mL) was added HCl (4 M, 1.92 mL). The mixture was stirred at room temperature overnight. Solids crashed out and were filtered off and washed with EtOAc. The solids were air dried to afford 1-[6-(1-methylpyrazol-4-yl)pyrazolo[1,5-a]pyrazin-4-yl]piperidin-3-amine hydrochloride (500 mg, 94% yield) as a brown solid. The solid was assumed to have 80% purity.

### Synthesis of N-[1-[6-(1-methylpyrazol-4-yl)pyrazolo[1,5-a]pyrazin-4-yl]-3-piperidyl]prop-2-enamide

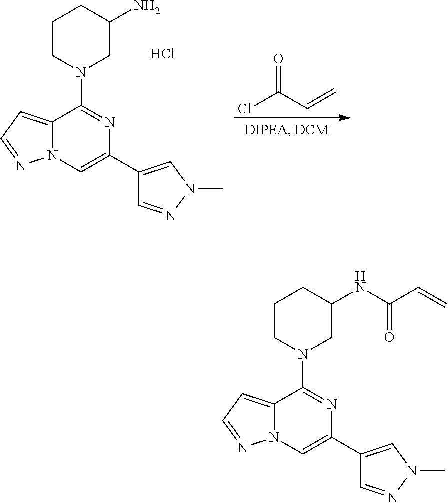

To a solution of 1-[6-(1-methylpyrazol-4-yl)pyrazolo[1,5-a]pyrazin-4-yl]piperidin-3-amine hydrochloride (75 mg, 180 μmol) in DCM (1.8 mL) was added N-ethyl-N-isopropyl-propan-2-amine (93 mg, 719 μmol, 126 μL) and prop-2-enoyl chloride (18 mg, 198 μmol, 16 μL) in that order. The reaction mixture was stirred at room temperature overnight. The material was concentrated and purified via reverse phase purification (Column: Waters XSelect CSH Prep C18 5 um OBD 19×100 mm; Condition: 5-45% Acetonitrile in 0.1% v/v Ammonium carbonate/water; Flowrate: 30 mL/min) to afford 30.5 mg (49% yield). LCMS m/z=352.2 (M+H)+. ¹H NMR (500 MHz, DMSO-d₆) δ ppm 1.19-1.30 (m, 1H) 1.53-1.69 (m, 2H) 1.81-2.01 (m, 2H) 3.06 (dd, J=12.82, 9.77 Hz, 1H) 3.23-3.27 (m, 1H) 3.87 (s, 3H) 3.91-3.97 (m, 1H) 4.22-4.47 (m, 2H) 5.57-5.66 (m, 1H) 6.18 (s, 1H) 6.23-6.34 (m, 1H) 7.06 (d, J=2.44 Hz, 1H) 7.89-8.03 (m, 1H) 8.19-8.27 (m, 2H) 8.40-8.53 (m, 1H).

### Example 21: N-[1-[6-(1-methylpyrazol-4-yl)pyrazolo[1,5-a]pyrazin-4-yl]-3-piperidyl]prop-2-ynamide

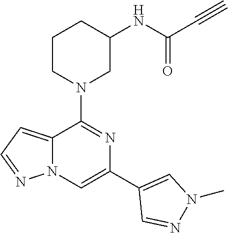 [SMILES: C#CC(=O)NC1CCCN(c2nc(-c3cnn(C)c3)cn3nccc23)C1]

### Synthesis of N-[1-[6-(1-methylpyrazol-4-yl)pyrazolo[1,5-a]pyrazin-4-yl]-3-piperidyl]prop-2-ynamide

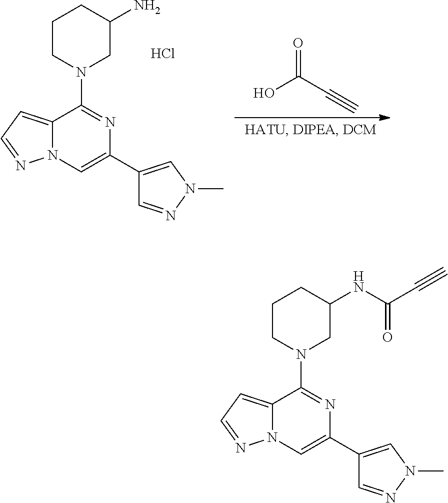

To a solution of 1-[6-(1-methylpyrazol-4-yl)pyrazolo[1,5-a]pyrazin-4-yl]piperidin-3-amine hydrochloride (75 mg, 180 μmol) in DCM (1.5 mL) was added N-ethyl-N-isopropyl-propan-2-amine (86 mg, 669 μmol, 117 μL) and HATU (68 mg, 178 μmol) in that order. The reaction mixture was stirred for 15 min and to it was then added prop-2-ynoic acid (15 mg, 216 μmol, 13 μL) and the resulting mixture was stirred at RT overnight. The material was concentrated and purified via reverse phase purification (Column: Waters XSelect CSH Prep C18 5 um OBD 19×100 mm; Condition: 5-45% Acetonitrile in 0.1% v/v Ammonium carbonate/water; Flowrate: 30 mL/min) to afford 14.8 mg (24% yield). LCMS m/z=350.1 (M+H)⁺. ¹H NMR (500 MHz, DMSO-d₆) δ ppm 1.22-1.43 (m, 1H) 1.51-1.68 (m, 2H) 1.78-1.99 (m, 2H) 3.04 (dd, J=12.82, 9.16 Hz, 1H) 3.18-3.26 (m, 1H) 3.87 (s, 3H) 3.90-3.99 (m, 1H) 4.22-4.41 (m, 2H) 6.96 (d, J=1.83 Hz, 1H) 7.90-8.07 (m, 1H) 8.20 (s, 1H) 8.44-8.56 (m, 1H) 8.92 (d, J=7.32 Hz, 1H). One proton signal is obscured by residual water in the deuterated solvent.

### Example 22: 4-chloro-N-[1-[6-(1-methylpyrazol-4-yl)pyrazolo[1,5-a]pyrazin-4-yl]-3-piperidyl]but-2-enamide

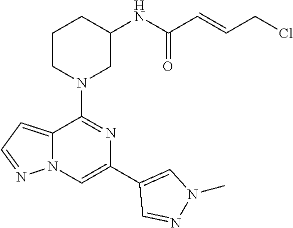 [SMILES: Cn1cc(-c2cn3nccc3c(N3CCCC(NC(=O)/C=C/CCl)C3)n2)cn1]

### Synthesis of 4-chloro-N-[1-[6-(1-methylpyrazol-4-yl)pyrazolo[1,5-a]pyrazin-4-yl]-3-piperidyl]but-2-enamide

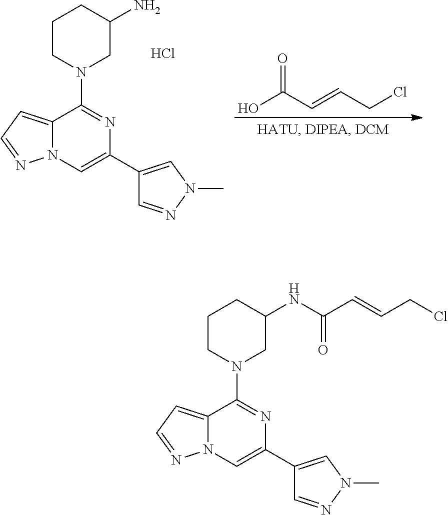 [SMILES: Cn1cc(-c2cn3nccc3c(N3CCCC(N)C3)n2)cn1 ; Cl ; O=C(O)/C=C/CCl ; Cn1cc(-c2cn3nccc3c(N3CCCC(NC(=O)/C=C/CCl)C3)n2)cn1]

To a solution of 1-[6-(1-methylpyrazol-4-yl)pyrazolo[1,5-a]pyrazin-4-yl]piperidin-3-amine hydrochloride (75 mg, 180 μmol) in DCM (1.5 mL) was added N-ethyl-N-isopropyl-propan-2-amine (86 mg, 669 μmol, 117 μL) and HATU (68 mg, 178 μmol) in that order. The reaction mixture was stirred for 15 min and to it was then added 4-chlorobut-2-enoic acid (26 mg, 216 μmol) and the resulting mixture was stirred at RT overnight. The material was concentrated and purified via reverse phase purification (Column: Waters XSelect CSH Prep C18 5 um OBD 19×100 mm; Condition: 5-55% Acetonitrile in 0.1% v/v Ammonium carbonate/water; Flowrate: 30 mL/min) to afford 5.7 mg (8% yield). LCMS m/z=400.2 (M+H)+. ¹H NMR (500 MHz, DMSO-d₆) δ: 8.51-8.43 (m, 1H), 8.30 (d, J=7.3 Hz, 1H), 8.25-8.17 (m, 1H), 8.02-7.89 (m, 1H), 7.09-6.98 (m, 1H), 6.75 (td, J=6.1, 14.6 Hz, 1H), 6.31-6.19 (m, 1H), 4.37 (dd, J=1.2, 6.1 Hz, 3H), 4.32-4.19 (m, 1H), 4.02-3.91 (m, 1H), 3.87 (s, 3H), 3.31-3.22 (m, 1H), 3.15-3.00 (m, 1H), 2.03-1.90 (m, 1H), 1.86 (br d, J=3.1 Hz, 1H), 1.70-1.54 (m, 2H), 1.33-1.17 (m, 1H).

### Example 23: N-methyl-N-[1-[7-(1-methylpyrazol-4-yl)imidazo[1,2-c]pyrimidin-5-yl]-3-piperidyl]prop-2-ynamide

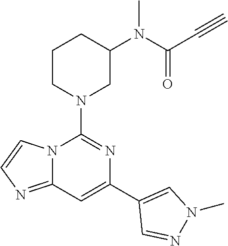 [SMILES: C#CC(=O)N(C)C1CCCN(c2nc(-c3cnn(C)c3)cc3nccn23)C1]

### Synthesis of tert-butyl N-methyl-N-[1-[7-(1-methylpyrazol-4-yl)imidazo[1,2-c]pyrimidin-5-yl]-3-piperidyl]carbamate

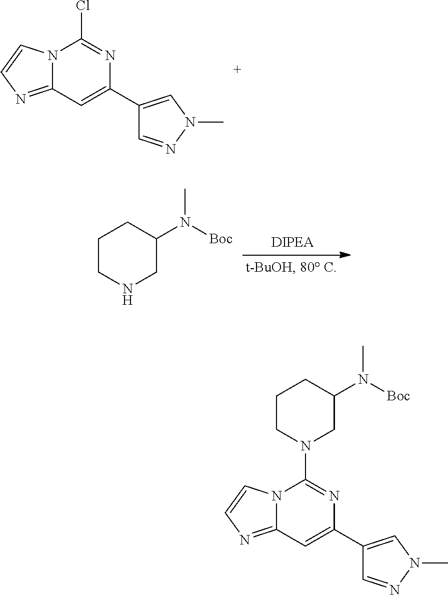

To a solution of 5-chloro-7-(1-methylpyrazol-4-yl)imidazo[1,2-c]pyrimidine (1.00 g, 4.28 mmol) in t-BuOH (8.6 mL) was added N-ethyl-N-isopropyl-propan-2-amine (830 mg, 6.42 mmol, 1.1 mL) and tert-butyl N-methyl-N-(3-piperidyl)carbamate (945 mg, 4.41 mmol) in that order. The reaction mixture was stirred at 80° C. overnight. The material was concentrated and taken forward as crude assuming 100% yield. LCMS m/z=412.0 (M+H)+.

### Synthesis of N-methyl-1-[7-(1-methylpyrazol-4-yl)imidazo[1,2-c]pyrimidin-5-yl]piperidin-3-amine hydrochloride

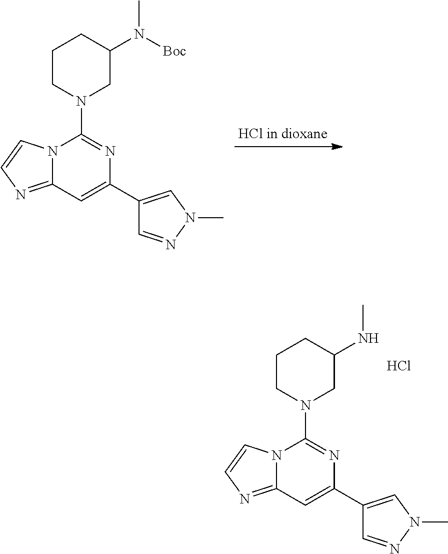 [SMILES: CN(C(=O)OC(C)(C)C)C1CCCN(c2nc(-c3cnn(C)c3)cc3nccn23)C1 ; CNC1CCCN(c2nc(-c3cnn(C)c3)cc3nccn23)C1 ; Cl]

To a solution of tert-butyl N-methyl-N-[1-[7-(1-methylpyrazol-4-yl)imidazo[1,2-c]pyrimidin-5-yl]-3-piperidyl]carbamate (1.76 g, 4.28 mmol) in dioxane (8.56 mL) was added HCl (4 M, 6.42 mL). The mixture was stirred at room temperature overnight. Solids crashed out overnight and were filtered off and washed with EtOAc. The solids were air dried to afford N-methyl-1-[7-(1-methylpyrazol-4-yl)imidazo[1,2-c]pyrimidin-5-yl]piperidin-3-amine hydrochloride (1.94 g, 3.90 mmol, 91% yield) as an off white solid. The solid was assumed to have 70% purity. LCMS m/z=312.1 (M+H)+.

### Synthesis of N-methyl-N-[1-[7-(1-methylpyrazol-4-yl)imidazo[1,2-c]pyrimidin-5-yl]-3-piperidyl]prop-2-ynamide

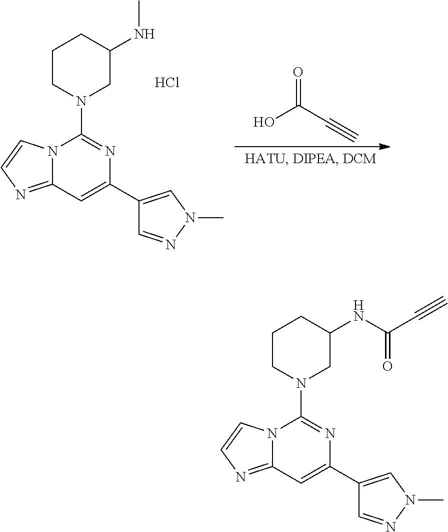

To a solution of N-methyl-1-[7-(1-methylpyrazol-4-yl)imidazo[1,2-c]pyrimidin-5-yl]piperidin-3-amine hydrochloride (100 mg, 201 μmol) in DCM (1 mL) was added N-ethyl-N-isopropyl-propan-2-amine (130 mg, 1.01 mmol, 176 μL), prop-2-ynoic acid (18 mg, 262 μmol, 16 μL) in that order. To the vial was then added HATU (100 mg, 262 μmol) and the resulting mixture was stirred at RT overnight. The material was concentrated and purified via reverse phase purification (Column: Waters XSelect CSH Prep C18 5 um OBD 19×100 mm; Condition: 5-45% Acetonitrile in 0.1% v/v Ammonium carbonate/water; Flowrate: 30 mL/min) to afford 33.4 mg (46% yield). LCMS m/z=364.3 (M+H)+.

### Example 24: N-methyl-N-[1-[6-(1-methylpyrazol-4-yl)pyrazolo[1,5-a]pyrazin-4-yl]-3-piperidyl]prop-2-ynamide

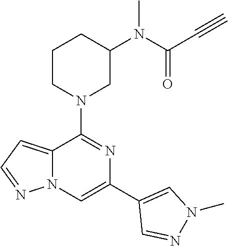 [SMILES: C#CC(=O)N(C)C1CCCN(c2nc(-c3cnn(C)c3)cn3nccc23)C1]

### Synthesis of tert-butyl N-methyl-N-[1-[6-(1-methylpyrazol-4-yl)pyrazolo[1,5-a]pyrazin-4-yl]-3-piperidyl]carbamate

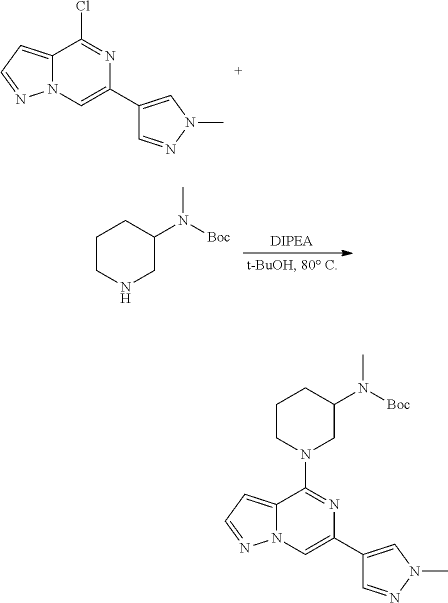

To a solution of 4-chloro-6-(1-methylpyrazol-4-yl)pyrazolo[1,5-a]pyrazine (400 mg, 1.71 mmol) in t-BuOH (3.42 mL) was added N-ethyl-N-isopropyl-propan-2-amine (332 mg, 2.57 mmol, 448 μL) and tert-butyl N-methyl-N-(3-piperidyl)carbamate (378 mg, 1.76 mmol) in that order. The vial was stirred at 80° C. overnight. The material was concentrated and taken forward as crude assuming 100% yield. LCMS m/z=412.1 (M+H)+.

### Synthesis of N-methyl-1-[6-(1-methylpyrazol-4-yl)pyrazolo[1,5-a]pyrazin-4-yl]piperidin-3-amine hydrochloride

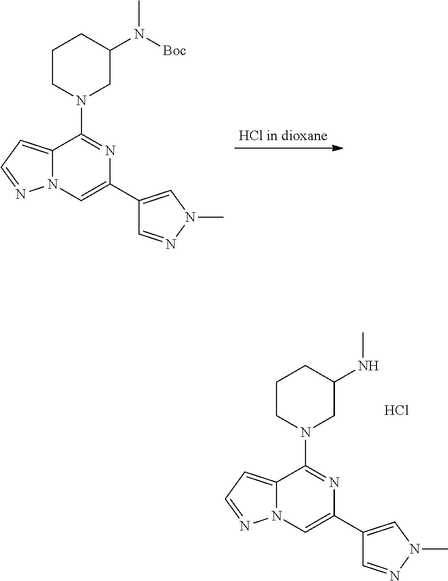 [SMILES: CN(C(=O)OC(C)(C)C)C1CCCN(c2nc(-c3cnn(C)c3)cn3nccc23)C1 ; CNC1CCCN(c2nc(-c3cnn(C)c3)cn3nccc23)C1 ; Cl]

To a solution of tert-butyl N-methyl-N-[1-[6-(1-methylpyrazol-4-yl)pyrazolo[1,5-a]pyrazin-4-yl]-3-piperidyl]carbamate (704 mg, 1.71 mmol) in dioxane (8.6 mL) was added HCl (4 M, 2.57 mL). The mixture was stirred at room temperature overnight. Solids crashed out overnight and were filtered off and washed with EtOAc. The solids were air dried to afford N-methyl-1-[6-(1-methylpyrazol-4-yl)pyrazolo[1,5-a]pyrazin-4-yl]piperidin-3-amine hydrochloride (651 mg, 88% yield) as an off white solid. The solid was assumed to have 80% purity. LCMS m/z=312.1 (M+H)+.

### Synthesis of N-methyl-N-[1-[6-(1-methylpyrazol-4-yl)pyrazolo[1,5-a]pyrazin-4-yl]-3-piperidyl]prop-2-ynamide

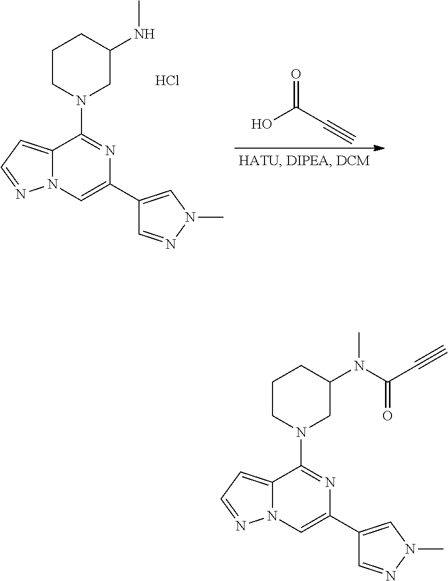

To a solution of 1 N-methyl-1-[6-(1-methylpyrazol-4-yl)pyrazolo[1,5-a]pyrazin-4-yl]piperidin-3-amine hydrochloride (100 mg, 230 μmol) in DCM (1 mL) was added prop-2-ynoic acid (21 mg, 299 μmol, 18 μL), N-ethyl-N-isopropyl-propan-2-amine (86 mg, 669 μmol, 117 μL) and HATU (114 mg, 299 μmol) in that order. The reaction mixture was stirred at room temperature overnight. The material was concentrated and purified via reverse phase purification (Column: Waters XSelect CSH Prep C18 5 um OBD 19×100 mm; Condition: 5-50% Acetonitrile in 0.1% v/v Ammonium carbonate/water; Flowrate: 30 mL/min) to afford 1.9 mg (2% yield). LCMS m/z=364.2 (M+H)+. ¹H NMR (500 MHz, DMSO-d₆) δ ppm 1.59-1.75 (m, 1H) 1.78-2.05 (m, 4H) 2.90 (s, 2H), 2.99-3.11 (m, 1H) 3.14-3.18 (m, 1H) 3.87 (d, J=1.83 Hz, 3H) 4.29-4.56 (m, 4H) 6.95 (d, J=2.44 Hz, 1H) 7.90-8.04 (m, 2H) 8.17 (d, J=17.70 Hz, 1H) 8.46-8.59 (m, 1H).

### Example 25: N-((1-(6-(1-methyl-1H-pyrazol-4-yl)pyrazolo[1,5-a]pyrazin-4-yl)piperidin-3-yl)methyl)propiolamide

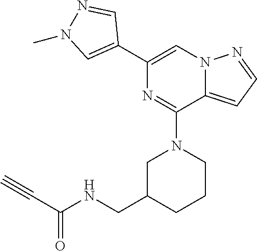 [SMILES: C#CC(=O)NCC1CCCN(c2nc(-c3cnn(C)c3)cn3nccc23)C1]

### Synthesis of tert-butyl N-[[1-[6-(1-methylpyrazol-4-yl)pyrazolo[1,5-a]pyrazin-4-yl]-3-piperidyl]methyl]carbamate

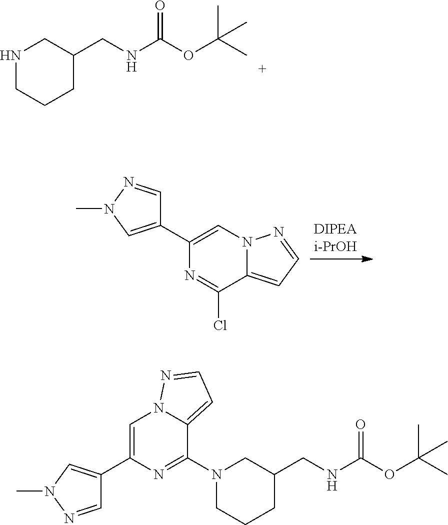 [SMILES: CC(C)(C)OC(=O)NCC1CCCNC1 ; Cn1cc(-c2cn3nccc3c(Cl)n2)cn1 ; Cn1cc(-c2cn3nccc3c(N3CCCC(CNC(=O)OC(C)(C)C)C3)n2)cn1]

A suspension of tert-butyl N-(3-piperidylmethyl)carbamate (229 mg, 1.07 mmol), 4-chloro-6-(1-methylpyrazol-4-yl)pyrazolo[1,5-a]pyrazine (250 mg, 1.07 mmol) and DIPEA (277 mg, 2.14 mmol, 374 μL) in isopropanol (4 mL) was heated to reflux for 17 h. The reaction mixture was cooled to RT and concentrated in vacuo. The residue was taken up in EtOAc and washed with water and brine. The organic layer was dried (MgSO₄), filtered and concentrated in vacuo. The residue was purified by silica-gel chromatography (0-100% EtOAc in heptanes). tert-Butyl N-[[1-[6-(1-methylpyrazol-4-yl)pyrazolo[1,5-a]pyrazin-4-yl]-3-piperidyl]methyl]carbamate (379 mg, 86% yield) was obtained as a pale yellow solid. LCMS: m/z=412.3 (M+H)⁺. ¹H NMR (500 MHz, CHLOROFORM-d) δ ppm 8.09 (s, 1H), 7.82-7.96 (m, 3H), 6.69 (br s, 1H), 4.81 (br s, 1H), 4.38 (br d, J=13.4 Hz, 2H), 3.98 (s, 3H), 3.29 (br t, J=11.0 Hz, 1H), 3.15 (br s, 2H), 3.09 (br dd, J=12.8, 9.8 Hz, 1H), 1.95 (br d, J=12.2 Hz, 2H), 1.87 (dt, J=13.4, 3.7 Hz, 1H), 1.68-1.78 (m, 1H), 1.45-1.53 (m, 9H), 1.33-1.42 (m, 1H).

### Synthesis of [1-[6-(1-methylpyrazol-4-yl)pyrrolo[2,1-f][1,2,4]triazin-4-yl]-3-piperidyl]methanamine (Hydrochloride)

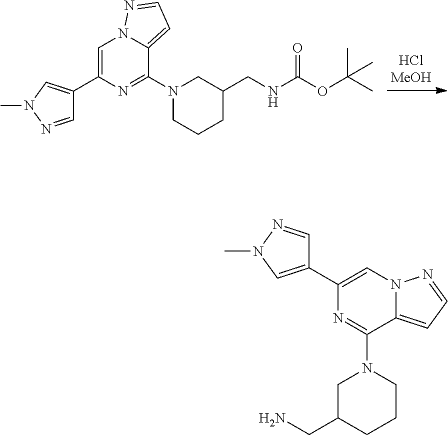

A solution of tert-butyl N-[[1-[6-(1-methylpyrazol-4-yl)pyrrolo[2,1-f][1,2,4]triazin-4-yl]-3-piperidyl]methyl]carbamate (150 mg, 365 μmol) in dry methanol (1 mL) was treated with hydrochloric acid (4 M solution in dioxane, 1 mL). The resulting mixture was stirred at RT for 1 h and was concentrated in vacuo. [1-[6-(1-methylpyrazol-4-yl)pyrrolo[2,1-f][1,2,4]triazin-4-yl]-3-piperidyl]methanamine hydrochloride was obtained as an off-white solid. LCMS: m/z=312.3 (M+H)+.

### Synthesis of N-[[1-[6-(1-methylpyrazol-4-yl)pyrazolo[1,5-a]pyrazin-4-yl]-3-piperidyl]methyl]prop-2-ynamide

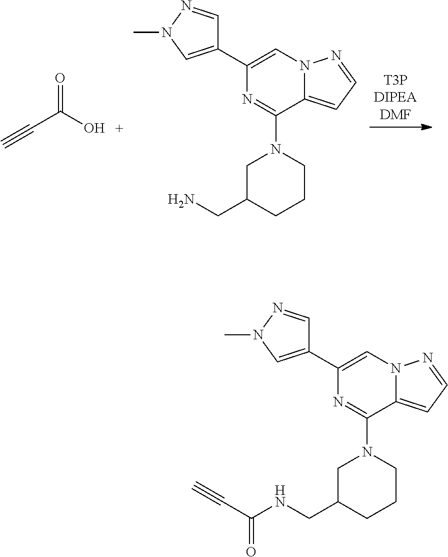

To a suspension of crude [1-[6-(1-methylpyrazol-4-yl)pyrazolo[1,5-a]pyrazin-4-yl]-3-piperidyl]methanamine hydrochloride (50 mg, 161 μmol) in dry DMF (1 mL) under an atmosphere of nitrogen at 0° C. was added DIPEA (62 mg, 482 μmol, 84 μL) followed by prop-2-ynoic acid (17 mg, 241 μmol, 15 μL) and T3P (204 mg, 321 μmol, 217 μL, 50% in DMF). The resulting solution was stirred at RT for 1 h, quenched with saturated sodium bicarbonate solution and extracted with EtOAc. The organic layer was washed with water and brine, dried (MgSO₄), filtered and concentrated in vacuo. The residue was purified by silica-gel chromatography (0-10% MeOH in DCM). N-[[1-[6-(1-methylpyrazol-4-yl)pyrazolo[1,5-a]pyrazin-4-yl]-3-piperidyl]methyl]prop-2-ynamide (42.8 mg, 66% yield, 90% purity) was obtained as an orange oil. LCMS: m/z=364.2 (M+H)+. ¹H NMR (500 MHz, CHLOROFORM-d) δ ppm 8.07 (s, 1H), 7.83-7.88 (m, 2H), 7.79-7.83 (m, 1H), 6.65 (d, J=2.4 Hz, 1H), 6.45 (br s, 1H), 4.16-4.25 (m, 2H), 3.94-3.98 (m, 3H), 3.44-3.52 (m, 1H), 3.35-3.44 (m, 1H), 3.28-3.35 (m, 1H), 3.20-3.27 (m, 1H), 2.80-2.84 (m, 1H), 2.08 (quind, J=9.0, 9.0, 9.0, 9.0, 3.7 Hz, 1H), 1.91-2.00 (m, 1H), 1.78-1.86 (m, 1H), 1.61-1.78 (m, 1H), 1.38-1.50 (m, 1H).

### Example 26: (S)—N-((1-(6-(1-methyl-1H-pyrazol-4-yl)pyrazolo[1,5-a]pyrazin-4-yl)piperidin-2-yl)methyl)propiolamide and (R)—N-((1-(6-(1-methyl-1H-pyrazol-4-yl)pyrazolo[1,5-a]pyrazin-4-yl)piperidin-2-yl)methyl)propiolamide

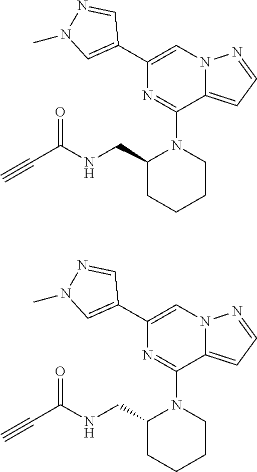 [SMILES: C#CC(=O)NC[C@@H]1CCCCN1c1nc(-c2cnn(C)c2)cn2nccc12 ; C#CC(=O)NC[C@H]1CCCCN1c1nc(-c2cnn(C)c2)cn2nccc12]

### Synthesis of tert-butyl N-[[1-[6-(1-methylpyrazol-4-yl)pyrazolo[1,5-a]pyrazin-4-yl]-2-piperidyl]methyl]carbamate

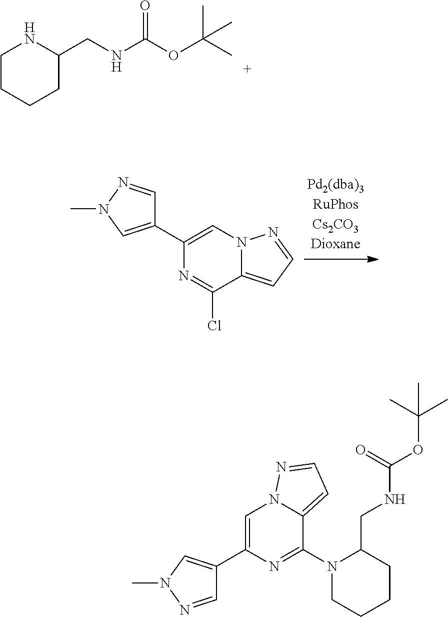 [SMILES: CC(C)(C)OC(=O)NCC1CCCCN1 ; Cn1cc(-c2cn3nccc3c(Cl)n2)cn1 ; Cn1cc(-c2cn3nccc3c(N3CCCCC3CNC(=O)OC(C)(C)C)n2)cn1]

A suspension of tert-butyl N-(2-piperidylmethyl)carbamate (302 mg, 1.41 mmol), 4-chloro-6-(1-methylpyrazol-4-yl)pyrazolo[1,5-a]pyrazine (300 mg, 1.28 mmol) and cesium carbonate (1.25 g, 3.84 mmol) in dioxane (5 mL) was sparged with nitrogen for 5 min. RuPhos (119 mg, 256 μmol) and Pd₂(dba)₃ (117 mg, 128 μmol) were added and the resulting mixture was heated to reflux overnight. Additional tert-butyl N-(2-piperidylmethyl)carbamate (302 mg, 1.41 mmol), RuPhos (119 mg, 256 μmol) and Pd₂(dba)₃ (117 mg, 128 μmol) were added and heating was continued for an additional 24 h. The reaction mixture was then cooled to RT, filtered through Celite rinsing with EtOAc, and the filtrate was concentrated in vacuo. The residue was purified by silica-gel chromatography (0-100% EtOAc in heptanes). tert-Butyl N-[[1-[6-(1-methylpyrazol-4-yl)pyrazolo[1,5-a]pyrazin-4-yl]-2-piperidyl]methyl]carbamate (267 mg, 51% yield) was obtained as a yellow foam. LCMS: m/z=412.3 (M+H)+. ¹H NMR (400 MHz, CHLOROFORM-d) δ ppm 8.09 (s, 1H), 7.82-7.95 (m, 3H), 6.69 (br s, 1H), 5.96 (br s, 1H), 4.90 (br s, 1H), 4.29-4.42 (m, 1H), 3.99 (s, 3H), 3.85 (br t, J=11.8 Hz, 1H), 3.35 (br d, J=13.6 Hz, 2H), 1.71-1.92 (m, 6H), 1.35 (s, 9H).

### Synthesis of [1-[6-(1-methylpyrazol-4-yl)pyrazolo[1,5-a]pyrazin-4-yl]-2-piperidyl]methanamine (Hydrochloride)

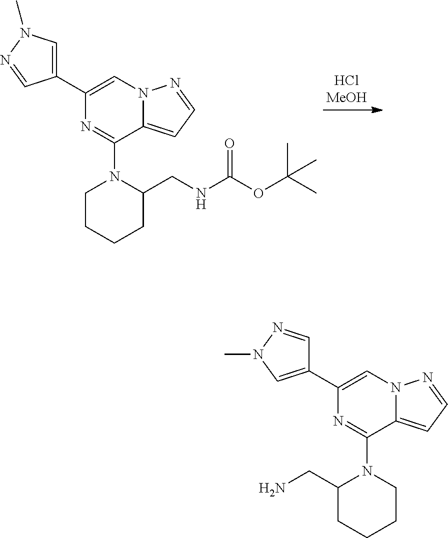

To a suspension of tert-butyl N-[[1-[6-(1-methylpyrazol-4-yl)pyrazolo[1,5-a]pyrazin-4-yl]-2-piperidyl]methyl]carbamate (150 mg, 365 μmol) in dry methanol (1 mL) was treated with hydrochloric acid (4 M solution in dioxane, 1 mL) and the resulting solution was stirred at RT for 2 h. A solid formed and the reaction mixture was concentrated in vacuo and the residue was used directly. [1-[6-(1-methylpyrazol-4-yl)pyrazolo[1,5-a]pyrazin-4-yl]-2-piperidyl]methanamine hydrochloride was obtained as a pale yellow solid. Quantitative yield assumed. LCMS: m/z=312.2 (M+H)+.

### Synthesis of N-[[1-[6-(1-methylpyrazol-4-yl)pyrazolo[1,5-a]pyrazin-4-yl]-2-piperidyl]methyl]prop-2-ynamide

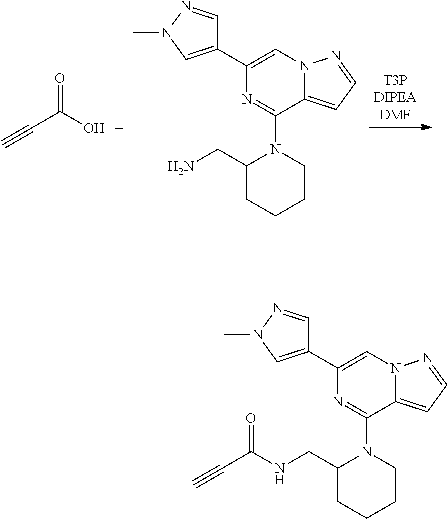

To a suspension of crude [1-[6-(1-methylpyrazol-4-yl)pyrazolo[1,5-a]pyrazin-4-yl]-2-piperidyl]methanamine hydrochloride (50 mg, 161 μmol) in dry DMF (1 mL) at 0° C. under nitrogen was added DIPEA (62 mg, 482 μmol, 84 μL) followed by prop-2-ynoic acid (17 mg, 241 μmol, 15 μL) and T3P (204 mg, 321 μmol, 217 μL, 50% in DMF). The resulting solution was stirred at RT for 1 h, quenched with saturated sodium bicarbonate solution and extracted with EtOAc. The organic layer was washed with water and brine, dried (MgSO₄), filtered and concentrated in vacuo. The residue was purified by silica gel chromatography (0-10% MeOH in DCM) and the product further purified by preparative TLC (7% MeOH in DCM). N-[[1-[6-(1-methylpyrazol-4-yl)pyrazolo[1,5-a]pyrazin-4-yl]-2-piperidyl]methyl]prop-2-ynamide (20 mg, 33% yield, 95% purity) was obtained as a brown solid. LCMS: m/z=364.2 (M+H)+. ¹H NMR (400 MHz, CHLOROFORM-d) δ ppm 8.56 (br s, 1H), 8.07-8.14 (m, 1H), 7.89 (d, J=2.3 Hz, 1H), 7.84 (s, 2H), 6.67 (d, J=2.0 Hz, 1H), 4.90-5.02 (m, 1H), 4.31 (br d, J=13.8 Hz, 1H), 4.05-4.19 (m, 1H), 3.92-4.00 (m, 3H), 3.42-3.52 (m, 1H), 3.36 (br s, 1H), 2.54 (s, 1H), 1.73-1.96 (m, 6H).

Chiral SFC purification (using CHIRALPAK AD-H 30×250 mm, 5 um column; Method: 30% MeOH with No Modifier in CO₂ (flow rate: 100 mL/min, ABPR 120 bar, MBPR 40 psi, column temp 40 deg C)) gave enantiomer E1 (as the first eluting peak, 7.9 mg, 100% ee). Rf=3.76 min and enantiomer E2 (as the second eluting peak, 7.8 mg, 95.90% ee). Rf=4.43 min.

### Example 27: N-[[1-[6-(1-methylpyrazol-4-yl)pyrazolo[1,5-a]pyrazin-4-yl]-2-piperidyl]methyl]prop-2-enamide

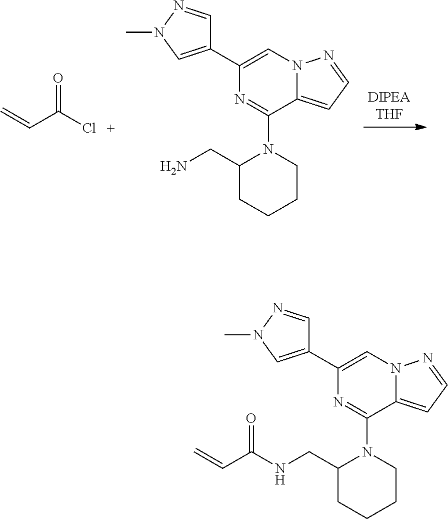

To a suspension of crude [1-[6-(1-methylpyrazol-4-yl)pyrazolo[1,5-a]pyrazin-4-yl]-2-piperidyl]methanamine hydrochloride (60 mg, 193 μmol) in dry THF (1 mL) under nitrogen and at 0° C. was added DIPEA (75 mg, 578 μmol, 101 μL) followed by prop-2-enoyl chloride (26 mg, 289 μmol, 24 μL). The resulting suspension was stirred at 0° C. for 10 min, quenched with saturated sodium bicarbonate solution and extracted with EtOAc. The organic layer was washed with brine, dried (MgSO₄), filtered and concentrated in vacuo. The residue was purified by silica-gel chromatography (0-10% MeOH in DCM) and further purified by preparative TLC (93:7 DCM/MeOH). N-[[1-[6-(1-methylpyrazol-4-yl)pyrazolo[1,5-a]pyrazin-4-yl]-2-piperidyl]methyl]prop-2-enamide (13.2 mg, 18% yield, 95% purity) was obtained as a brownish foam. LCMS: m/z=366.1 (M+H)+. ¹H NMR (400 MHz, CHLOROFORM-d) δ ppm 8.09 (s, 1H), 7.86-7.90 (m, 1H), 7.83-7.86 (m, 1H), 7.78 (s, 1H), 7.70-7.90 (br s, 1H), 6.67 (d, J=2.0 Hz, 1H), 6.08 (dd, J=17.1, 1.3 Hz, 1H), 5.79 (dd, J=17.1, 10.3 Hz, 1H), 5.43 (dd, J=10.4, 1.4 Hz, 1H), 4.98-5.08 (m, 1H), 4.27-4.40 (m, 1H), 4.08-4.22 (m, 1H), 3.93-4.00 (m, 3H), 3.44-3.53 (m, 1H), 3.28-3.44 (m, 1H), 1.72-1.96 (m, 6H).

### Example 28: (R)-6-(1-methyl-1H-pyrazol-4-yl)-4-((1-(vinylsulfonyl)azepan-4-yl)oxy)pyrazolo[1,5-a]pyrazine

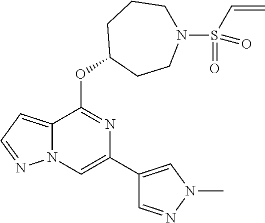 [SMILES: C=CS(=O)(=O)N1CCC[C@@H](Oc2nc(-c3cnn(C)c3)cn3nccc23)CC1]

### 1. Synthesis of tert-butyl (R)-4-((6-(1-methyl-1H-pyrazol-4-yl)pyrazolo[1,5-a]pyrazin-4-yl)oxy)azepane-1-carboxylate

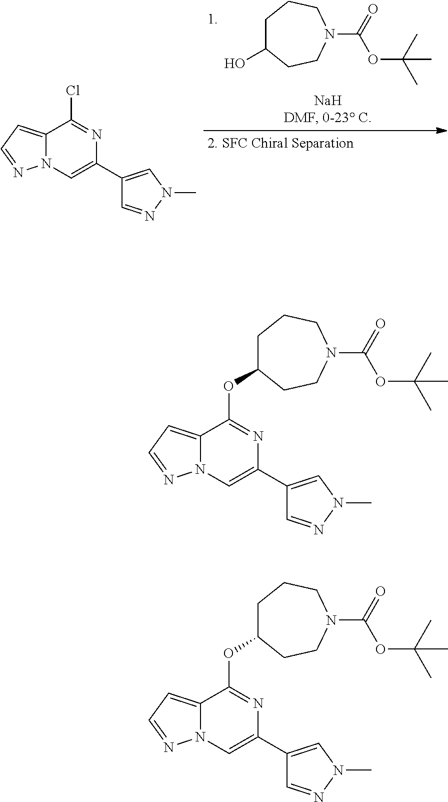 [SMILES: CC(C)(C)OC(=O)N1CCCC(O)CC1 ; Cn1cc(-c2cn3nccc3c(Cl)n2)cn1 ; Cn1cc(-c2cn3nccc3c(O[C@H]3CCCN(C(=O)OC(C)(C)C)CC3)n2)cn1 ; Cn1cc(-c2cn3nccc3c(O[C@@H]3CCCN(C(=O)OC(C)(C)C)CC3)n2)cn1]

A solution of tert-butyl 4-hydroxyazepane-1-carboxylate (710 mg, 3.30 mmol) in dry DMF (10 mL) was cooled in an ice bath. Then, sodium hydride (396 mg, 9.90 mmol, 60% purity) was added in 4 batches while stirring. Stirring in the ice bath was continued for 45 minutes during which a pale-yellow suspension formed. To this mixture was added 4-chloro-6-(1-methylpyrazol-4-yl)pyrazolo[1,5-a]pyrazine (700 mg, 3.00 mmol) as one batch. Stirring was continued at room temperature overnight. The mixture was diluted with ethyl acetate and then water was added carefully. The mixture was transferred to a separating funnel and the phases were separated. The aqueous phase was extracted with ethyl acetate once more and the combined organic phases were washed with brine, dried with Na₂SO₄, filtered, and concentrated. The residual material was purified on a 10 g silica column in 50% heptanes/ethyl acetate to give tert-butyl 4-[6-(1-methylpyrazol-4-yl)pyrazolo[1,5-a]pyrazin-4-yl]oxyazepane-1-carboxylate (1.30 g, yield: 95%) as a sticky pale-yellow gum. The racemic material was resolved by chiral SFC purification (CHIRALPAK AD-H 30×250 mm, 5 um, 25% IPA with 0.1% DEA in CO₂, flow rate: 100 mL/min, ABPR 120 bar, MBPR 60 psi, column temp 40 deg C) to afford (S)-tert-butyl 4-[6-(1-methylpyrazol-4-yl)pyrazolo[1,5-a]pyrazin-4-yl]oxyazepane-1-carboxylate (first eluting peak) (389 mg, yield: 63%) and (R)-tert-butyl 4-[6-(1-methylpyrazol-4-yl)pyrazolo[1,5-a]pyrazin-4-yl]oxyazepane-1-carboxylate (second eluting peak) (407 mg, yield: 66%) as off-white solids. The absolute stereochemistry of the product in these two peaks was later confirmed using commercially-available chiral tert-butyl (4S)-hydroxyazepane-1-carboxylate to synthesize a compound whose analytical data aligned with the first eluting peak. ESI-MS (M+H)⁺: 413.2.

### 2. Synthesis of (R)-4-(azepan-4-yloxy)-6-(1-methylpyrazol-4-yl)pyrazolo[1,5-a]pyrazine

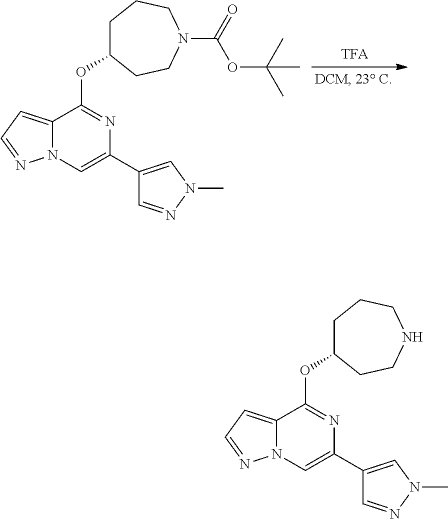 [SMILES: Cn1cc(-c2cn3nccc3c(O[C@@H]3CCCN(C(=O)OC(C)(C)C)CC3)n2)cn1 ; Cn1cc(-c2cn3nccc3c(O[C@@H]3CCCNCC3)n2)cn1]

To a solution of tert-butyl (4R)-[6-(1-methylpyrazol-4-yl)pyrazolo[1,5-a]pyrazin-4-yl]oxyazepane-1-carboxylate (407 mg, 987 μmol) in DCM (3 mL) is added TFA (2.25 g, 19.7 mmol, 1.51 mL) with stirring at room temperature. After stirring overnight the mixture was dissolved in MeOH and purified on a 5 g SCX column where the desired product was eluted with 2 M NH₃-MeOH to give 4-(azepan-4-yloxy)-6-(1-methylpyrazol-4-yl)pyrazolo[1,5-a]pyrazine (250 mg, yield: 77% yield) as a white solid. ESI-MS (M+H)⁺: 313.2.

### 3. Synthesis of (R)-6-(1-methyl-1H-pyrazol-4-yl)-N-((1-(vinylsulfonyl)piperidin-4-yl)methyl)pyrazolo[1,5-a]pyrazin-4-amine

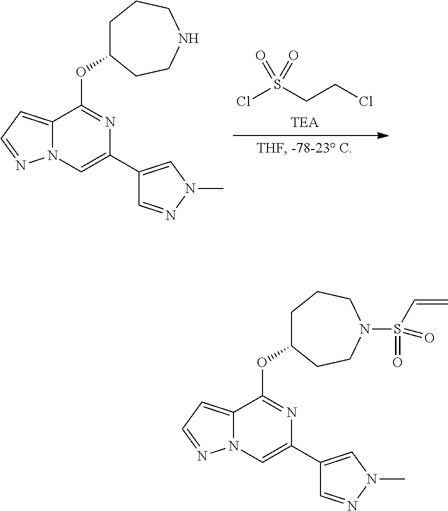 [SMILES: Cn1cc(-c2cn3nccc3c(O[C@@H]3CCCNCC3)n2)cn1 ; O=S(=O)(Cl)CCCl ; C=CS(=O)(=O)N1CCC[C@@H](Oc2nc(-c3cnn(C)c3)cn3nccc23)CC1]

To a solution of (4R)-(azepan-4-yloxy)-6-(1-methylpyrazol-4-yl)pyrazolo[1,5-a]pyrazine (30 mg, 96 μmol) in THF (1 mL) was added 2-chloroethanesulfonyl chloride (31 mg, 192 μmol, 20 μL) upon which precipitation occurred instantaneously. Then, TEA (39 mg, 384 μmol, 53 μL) was added with stirring at room temperature. After stirring overnight, the volatiles were removed and the residual material was re-dissolved in DMSO and purified via reverse phase HPLC (Waters XSelect CSH C18, 5 μm, 19 mm×100 mm column with mobile phase H₂O (A) and MeCN (B) and a gradient of 5-60% B (0.2% NH₄OH final v/v % modifier) with flow rate at 30 mL/min) to afford (R)-6-(1-methylpyrazol-4-yl)-4-(1-vinylsulfonylazepan-4-yl)oxy-pyrazolo[1,5-a]pyrazine (11 mg, yield: 26%) as a yellow oil. ESI-MS (M+H)⁺: 403.2. ¹H NMR (500 MHz, DMSO-d₆) δ 8.74 (s, 1H), 8.19 (s, 1H), 7.98-8.03 (m, 1H), 6.79-6.94 (m, 2H), 6.05-6.11 (m, 2H), 5.57 (tt, J=3.66, 7.63 Hz, 1H), 3.88 (s, 3H), 3.40-3.67 (m, 2H), 3.33-3.38 (m, 2H), 3.21-3.31 (m, 1H), 2.17-2.27 (m, 1H), 1.99-2.10 (m, 3H), 1.89-1.99 (m, 1H), 1.68-1.83 (m, 1H).

### Example 29: (R)-1-(4-((6-(1-methyl-1H-pyrazol-4-yl)pyrazolo[1,5-a]pyrazin-4-yl)oxy)azepan-1-yl)prop-2-en-1-one

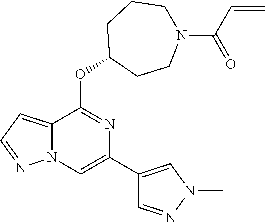 [SMILES: C=CC(=O)N1CCC[C@@H](Oc2nc(-c3cnn(C)c3)cn3nccc23)CC1]

### 1. Synthesis of (R)-1-(4-((6-(1-methyl-1H-pyrazol-4-yl)pyrazolo[1,5-a]pyrazin-4-yl)oxy)azepan-1-yl)prop-2-en-1-one

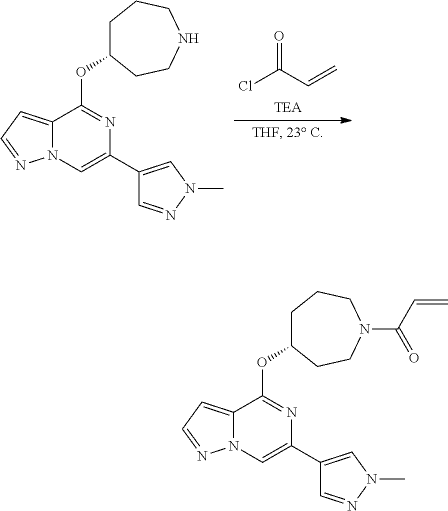

To a solution of (4R)-(azepan-4-yloxy)-6-(1-methylpyrazol-4-yl)pyrazolo[1,5-a]pyrazine (30 mg, 96 μmol) in THF (1 mL) was added prop-2-enoyl chloride (17 mg, 192 μmol, 16 μL) upon which precipitation occurred instantaneously. Then, TEA (19 mg, 192 μmol, 27 μL) was added with stirring at room temperature. After stirring overnight, the volatiles were removed and the residual material was re-dissolved in DMSO and purified via reverse phase HPLC (Waters XSelect CSH C18, 5 μm, 19 mm×100 mm column with mobile phase H₂O (A) and MeCN (B) and a gradient of 5-50% B (0.2% NH₄OH final v/v % modifier) with flow rate at 30 mL/min) to afford (R)-1-(4-((6-(1-methyl-1H-pyrazol-4-yl)pyrazolo[1,5-a]pyrazin-4-yl)oxy)azepan-1-yl)prop-2-en-1-one (9 mg, yield: 23%) as a clear oil. ESI-MS (M+H)⁺: 367.2. ¹H NMR (500 MHz, DMSO-d₆) δ 8.73 (s, 1H), 8.18 (d, J=18.92 Hz, 1H), 7.98-8.03 (m, 1H), 6.76-6.87 (m, 2H), 6.18 (ddd, J=2.44, 3.66, 16.48 Hz, 1H), 5.65-5.75 (m, 1H), 5.45-5.58 (m, 1H), 3.88 (d, J=1.83 Hz, 3H), 3.56-3.79 (m, 4H), 2.71-2.92 (m, 1H), 2.17-2.26 (m, 1H), 1.99-2.10 (m, 2H), 1.84-1.98 (m, 2H), 1.63-1.82 (m, 1H).

### Example 30: (R)-1-(4-((6-(1-methyl-1H-pyrazol-4-yl)pyrazolo[1,5-a]pyrazin-4-yl)oxy)azepan-1-yl)prop-2-yn-1-one

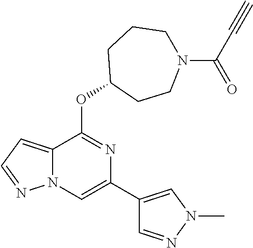 [SMILES: C#CC(=O)N1CCC[C@@H](Oc2nc(-c3cnn(C)c3)cn3nccc23)CC1]

### 1. Synthesis of (R)-1-(4-((6-(1-methyl-1H-pyrazol-4-yl)pyrazolo[1,5-a]pyrazin-4-yl)oxy)azepan-1-yl)prop-2-yn-1-one

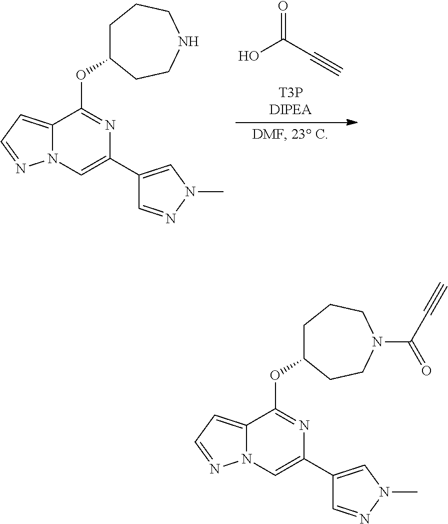

To a solution of (4R)-(azepan-4-yloxy)-6-(1-methylpyrazol-4-yl)pyrazolo[1,5-a]pyrazine (30 mg, 96 μmol) in DMF (1 mL) was added propiolic acid (13 mg, 192 μmol, 12 μL) followed by DIPEA (25 mg, 192 μmol, 34 μL) with stirring at room temperature. After stirring at room temperature for 5 minutes, T3P (122 mg, 192 μmol, 50% purity) was added dropwise with stirring. After stirring at room temperature for a further 3 hours, the mixture was diluted with ethyl acetate and washed with water. The organic phase was dried with sodium sulfate, filtered, and evaporated. The residual white solid is re-dissolved in DMSO and purified via reverse phase HPLC (Waters XSelect CSH C18, 5 μm, 19 mm×100 mm column with mobile phase H₂O (A) and MeCN (B) and a gradient of 5-55% B (0.2% NH₄OH final v/v % modifier) with flow rate at 30 mL/min) to afford (R)-1-(4-((6-(1-methyl-1H-pyrazol-4-yl)pyrazolo[1,5-a]pyrazin-4-yl)oxy)azepan-1-yl)prop-2-yn-1-one (24 mg, yield: 48%) as a white solid. ESI-MS (M+H)⁺: 365.2. ¹H NMR (500 MHz, DMSO-d₆) δ 8.70-8.81 (m, 1H), 8.19 (d, J=3.66 Hz, 1H), 7.95-8.08 (m, 2H), 6.74-6.88 (m, 1H), 5.45-5.63 (m, 1H), 3.88 (s, 3H), 3.75-3.84 (m, 2H), 3.48-3.66 (m, 2H), 2.19-2.29 (m, 1H), 2.10-2.20 (m, 1H), 1.85-2.09 (m, 4H), 1.67-1.84 (m, 1H).

### Example 31: (S)-1-(4-((6-(1-methyl-1H-pyrazol-4-yl)pyrazolo[1,5-a]pyrazin-4-yl)oxy)azepan-1-yl)prop-2-yn-1-one

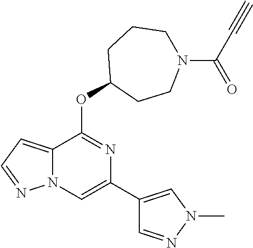 [SMILES: C#CC(=O)N1CCC[C@H](Oc2nc(-c3cnn(C)c3)cn3nccc23)CC1]

### 1. Synthesis of (S)-4-(azepan-4-yloxy)-6-(1-methylpyrazol-4-yl)pyrazolo[1,5-a]pyrazine

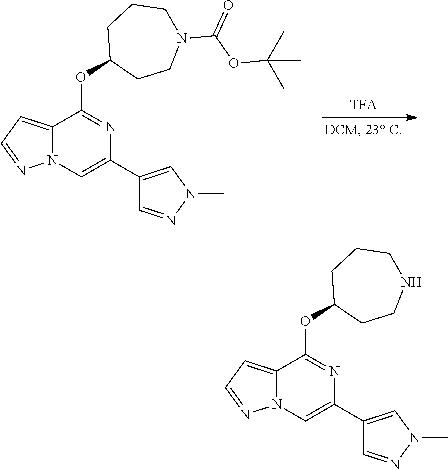 [SMILES: Cn1cc(-c2cn3nccc3c(O[C@H]3CCCN(C(=O)OC(C)(C)C)CC3)n2)cn1 ; Cn1cc(-c2cn3nccc3c(O[C@H]3CCCNCC3)n2)cn1]

To a solution of tert-butyl (4S)-4-[6-(1-methylpyrazol-4-yl)pyrazolo[1,5-a]pyrazin-4-yl]oxyazepane-1-carboxylate (389 mg, 944 μmol) in DCM (3 mL) was added TFA (2.15 g, 18.9 mmol, 1.44 mL) with stirring at room temperature. After stirring overnight the mixture was dissolved in MeOH and purified on a 5 g SCX column where the desired product is eluted with 2 M NH₃-MeOH to give 4-[(4S)-azepan-4-yl]oxy-6-(1-methylpyrazol-4-yl)pyrazolo[1,5-a]pyrazine (170 mg, yield: 55%) as a white solid. ESI-MS (M+H)⁺: 313.2.

### 2. Synthesis of (S)-1-(4-((6-(1-methyl-1H-pyrazol-4-yl)pyrazolo[1,5-a]pyrazin-4-yl)oxy)azepan-1-yl)prop-2-yn-1-one

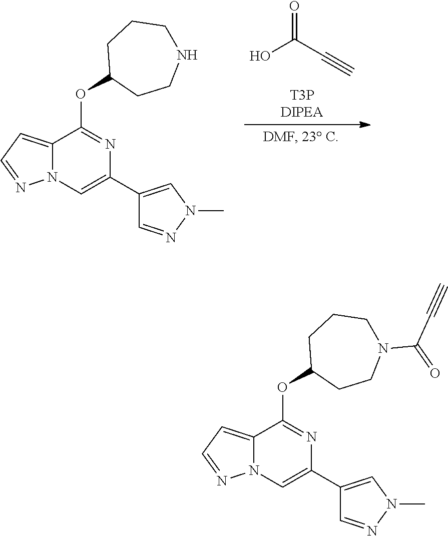

To a solution of (4S)-(azepan-4-yloxy)-6-(1-methylpyrazol-4-yl)pyrazolo[1,5-a]pyrazine (30 mg, 96 μmol) in DMF (1 mL) was added propiolic acid (13 mg, 192 μmol, 12 μL) followed by DIPEA (25 mg, 192 μmol, 34 μL) with stirring at room temperature. After stirring at room temperature for 5 minutes, T3P (122 mg, 192 μmol, 50% purity) was added dropwise with stirring. After stirring at room temperature for a further 3 hours, the mixture was diluted with ethyl acetate and washed with water. The organic phase was dried with sodium sulfate, filtered, and evaporated. The residual white solid is re-dissolved in DMSO and purified via reverse phase HPLC (Waters XSelect CSH C18, 5 μm, 19 mm×100 mm column with mobile phase H₂O (A) and MeCN (B) and a gradient of 5-55% B (0.2% NH₄OH final v/v % modifier) with flow rate at 30 mL/min) to afford (S)-1-(4-((6-(1-methyl-1H-pyrazol-4-yl)pyrazolo[1,5-a]pyrazin-4-yl)oxy)azepan-1-yl)prop-2-yn-1-one (18 mg, yield: 48%) as a yellow solid. ESI-MS (M+H)⁺: 365.2. ¹H NMR (500 MHz, DMSO-d₆) δ 8.74 (d, J=3.05 Hz, 1H), 8.19 (d, J=3.66 Hz, 1H), 7.98-8.05 (m, 2H), 6.79-6.87 (m, 1H), 5.46-5.64 (m, 1H), 3.88 (s, 3H), 3.75-3.84 (m, 2H), 3.50-3.67 (m, 2H), 2.12-2.29 (m, 2H), 1.87-2.11 (m, 4H), 1.67-1.84 (m, 1H).

### Example 32: (S)-1-(4-((6-(1-methyl-1H-pyrazol-4-yl)pyrazolo[1,5-a]pyrazin-4-yl)oxy)azepan-1-yl)prop-2-en-1-one

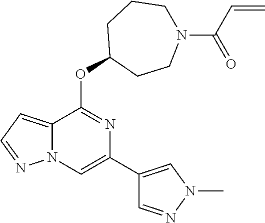 [SMILES: C=CC(=O)N1CCC[C@H](Oc2nc(-c3cnn(C)c3)cn3nccc23)CC1]

### 1. Synthesis of (S)-1-(4-((6-(1-methyl-1H-pyrazol-4-yl)pyrazolo[1,5-a]pyrazin-4-yl)oxy)azepan-1-yl)prop-2-en-1-one

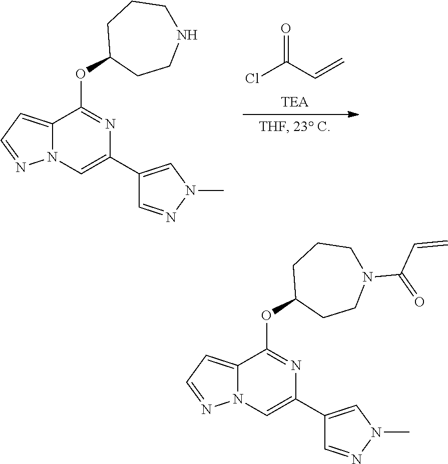

To a solution of (S)-4-(azepan-4-yloxy)-6-(1-methylpyrazol-4-yl)pyrazolo[1,5-a]pyrazine (30 mg, 96 μmol) in THF (1 mL) was added prop-2-enoyl chloride (17 mg, 192 μmol, 16 μL) upon which precipitation occurred instantaneously. Then, TEA (19 mg, 192 μmol, 27 μL) was added with stirring at room temperature. After stirring overnight, the volatiles were removed and the residual material was re-dissolved in DMSO and purified via reverse phase HPLC (Waters XSelect CSH C18, 5 μm, 19 mm×100 mm column with mobile phase H₂O (A) and MeCN (B) and a gradient of 5-50% B (0.2% NH₄OH final v/v % modifier) with flow rate at 30 mL/min) to afford (S)-1-(4-((6-(1-methyl-1H-pyrazol-4-yl)pyrazolo[1,5-a]pyrazin-4-yl)oxy)azepan-1-yl)prop-2-en-1-one (25 mg, yield: 67%) as a clear oil. ESI-MS (M+H)⁺: 367.2. ¹H NMR (500 MHz, DMSO-d₆) δ 8.73 (d, J=1.22 Hz, 1H), 8.18 (d, J=18.92 Hz, 1H), 7.96-8.02 (m, 2H), 6.76-6.87 (m, 2H), 6.18 (ddd, J=2.44, 3.66, 16.48 Hz, 1H), 5.66-5.72 (m, 1H), 5.45-5.58 (m, 1H), 3.88 (d, J=1.83 Hz, 3H), 3.55-3.79 (m, 4H), 2.18-2.25 (m, 1H), 1.99-2.07 (m, 2H), 1.85-1.98 (m, 2H), 1.69-1.79 (m, 1H).

### Example 33: (S)-6-(1-methyl-1H-pyrazol-4-yl)-4-((1-(vinylsulfonyl)azepan-4-yl)oxy)pyrazolo[1,5-a]pyrazine

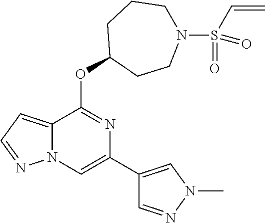 [SMILES: C=CS(=O)(=O)N1CCC[C@H](Oc2nc(-c3cnn(C)c3)cn3nccc23)CC1]

### Synthesis of (S)-6-(1-methyl-1H-pyrazol-4-yl)-N-((1-(vinylsulfonyl)piperidin-4-yl)methyl)pyrazolo[1,5-a]pyrazin-4-amine

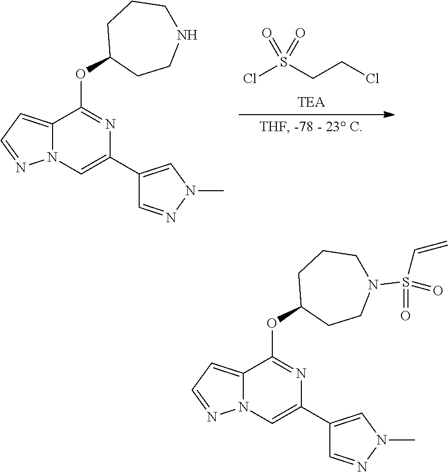 [SMILES: Cn1cc(-c2cn3nccc3c(O[C@H]3CCCNCC3)n2)cn1 ; O=S(=O)(Cl)CCCl ; C=CS(=O)(=O)N1CCC[C@H](Oc2nc(-c3cnn(C)c3)cn3nccc23)CC1]

To a solution of 4-(azepan-4-yloxy)-6-(1-methylpyrazol-4-yl)pyrazolo[1,5-a]pyrazine (30 mg, 96 μmol) in THF (1 mL) was added 2-chloroethanesulfonyl chloride (31 mg, 192 μmol, 20 μL) upon which precipitation occurred instantaneously. Then, TEA (39 mg, 384 μmol, 53 μL) was added with stirring at room temperature. After stirring overnight, the volatiles were removed and the residual material was re-dissolved in DMSO and purified via reverse phase HPLC (Waters XSelect CSH C18, 5 μm, 19 mm×100 mm column with mobile phase H₂O (A) and MeCN (B) and a gradient of 5-60% B (0.2% NH₄OH final v/v % modifier) with flow rate at 30 mL/min) to afford (S)-6-(1-methylpyrazol-4-yl)-4-(1-vinylsulfonylazepan-4-yl)oxy-pyrazolo[1,5-a]pyrazine (7 mg, yield: 16%) as a yellow oil. ESI-MS (M+H)⁺: 403.2. ¹H NMR (500 MHz, DMSO-d₆) δ 8.74 (s, 1H), 8.19 (s, 1H), 7.98-8.04 (m, 2H), 6.82-6.91 (m, 2H), 6.09 (d, J=16.48 Hz, 1H), 6.06 (d, J=10.38 Hz, 1H), 5.57 (tt, J=3.66, 7.63 Hz, 1H), 3.88 (s, 3H), 3.43-3.52 (m, 1H), 2.17-2.25 (m, 1H), 2.00-2.09 (m, 4H), 1.88-1.98 (m, 2H), 1.69-1.82 (m, 2H).

### Example 34: (S) or (R)-1-(4-((7-(1-methyl-1H-pyrazol-4-yl)imidazo[1,2-c]pyrimidin-5-yl)oxy)azepan-1-yl)prop-2-en-1-one

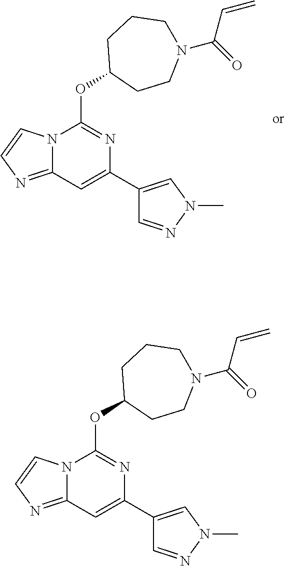 [SMILES: C=CC(=O)N1CCC[C@@H](Oc2nc(-c3cnn(C)c3)cc3nccn23)CC1 ; C=CC(=O)N1CCC[C@H](Oc2nc(-c3cnn(C)c3)cc3nccn23)CC1]

### 1. Synthesis of tert-butyl (R) and (S)-4-((7-(1-methyl-1H-pyrazol-4-yl)imidazo[1,2-c]pyrimidin-5-yl)oxy)azepane-1-carboxylate

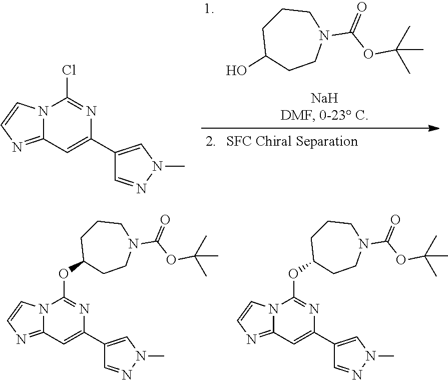 [SMILES: Cn1cc(-c2cc3nccn3c(Cl)n2)cn1 ; CC(C)(C)OC(=O)N1CCCC(O)CC1 ; Cn1cc(-c2cc3nccn3c(O[C@H]3CCCN(C(=O)OC(C)(C)C)CC3)n2)cn1 ; Cn1cc(-c2cc3nccn3c(O[C@@H]3CCCN(C(=O)OC(C)(C)C)CC3)n2)cn1]

A solution of tert-butyl 4-hydroxyazepane-1-carboxylate (355 mg, 1.65 mmol) in dry DMF (5 mL) was cooled in an ice bath. Then, sodium hydride (198 mg, 4.95 mmol, 60% purity) is added in 4 batches while stirring. Stirring in the ice bath was continued for 45 minutes during which a pale-yellow suspension formed. To this mixture was added 5-chloro-7-(1-methylpyrazol-4-yl)imidazo[1,2-c]pyrimidine (350 mg, 1.50 mmol) as one batch. Stirring at room temperature was continued overnight. The mixture was diluted with ethyl acetate and then water was added carefully. The mixture was transferred to a separating funnel and the phases were separated. The aqueous phase was extracted with ethyl acetate once more and the combined organic phases were washed with brine, dried with Na₂SO₄, filtered, and concentrated. The residual material was purified on a 10 g silica column in 50% heptanes/ethyl acetate to give tert-butyl 4-[7-(1-methylpyrazol-4-yl)imidazo[1,2-c]pyrimidin-5-yl]oxyazepane-1-carboxylate (520 mg, yield: 84%) as a sticky pale-yellow gum. 450 mg of the racemic material was resolved by chiral SFC purification (CHIRALPAK AD-H 30×250 mm, 5 um, 30% IPA with 0.1% DEA in CO₂, flow rate: 100 mL/min, ABPR 120 bar, MBPR 60 psi, column temp 40 deg C) to afford the two products as a first eluting peak (E1) Peak 1 (172 mg, yield: 76%) and a second eluting peak (E2) Peak 2 (171 mg, yield: 76%) as off-white solids. ESI-MS (M+H)⁺: 413.3. The absolute stereochemistry of the product in each peak was not assigned.

### 2. Synthesis of (R) or (S)-5-(azepan-4-yloxy)-7-(1-methyl-1H-pyrazol-4-yl)imidazo[1,2-c]pyrimidine

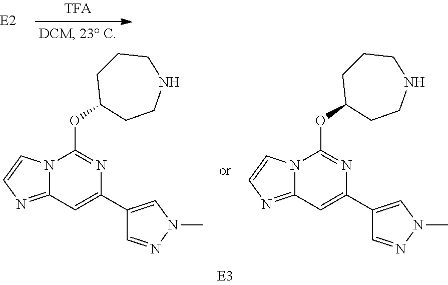 [SMILES: Cn1cc(-c2cc3nccn3c(O[C@@H]3CCCNCC3)n2)cn1 ; Cn1cc(-c2cc3nccn3c(O[C@H]3CCCNCC3)n2)cn1]

To a solution of E2 tert-butyl (4R) or (4S)-4-[7-(1-methylpyrazol-4-yl)imidazo[1,2-c]pyrimidin-5-yl]oxyazepane-1-carboxylate (172 mg, 416 μmol) in DCM (3 mL) was added TFA (949 mg, 8.33 mmol, 637 μL) with stirring at room temperature. After stirring overnight the reaction was dissolved in MeOH and purified on a 10 g SCX column where the desired product was eluted with 2 M NH₃-MeOH to give E3,5-[(4R) or (4S)-azepan-4-yl]oxy-7-(1-methylpyrazol-4-yl)imidazo[1,2-c]pyrimidine (95 mg, yield: 69%) as a white solid. ESI-MS (M+H)⁺: 313.2.

### 3. Synthesis of (R) or (S)-1-(4-((7-(1-methyl-1H-pyrazol-4-yl)imidazo[1,2-c]pyrimidin-5-yl)oxy)azepan-1-yl)prop-2-en-1-one

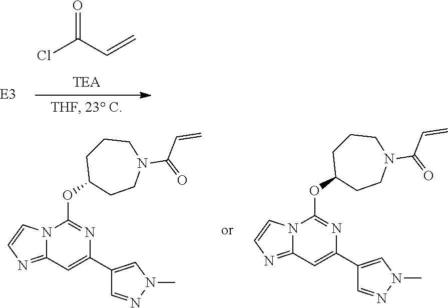

To a solution of E3,5-(4R) or (4S)-(azepan-4-yloxy)-7-(1-methylpyrazol-4-yl)imidazo[1,2-c]pyrimidine (30 mg, 96 μmol) (single enantiomer; chirality assigned arbitrarily) in THF (1 mL) was added prop-2-enoyl chloride (17 mg, 192 μmol, 16 μL) upon which precipitation occurred instantaneously. Then, TEA (19 mg, 192 μmol, 27 μL) was added with stirring at room temperature. After stirring overnight, the volatiles were removed and the residual material was re-dissolved in DMSO and purified via reverse phase HPLC (Waters XSelect CSH C18, 5 μm, 19 mm×100 mm column with mobile phase H₂O (A) and MeCN (B) and a gradient of 5-35% B (0.2% NH₄OH final v/v % modifier) with flow rate at 30 mL/min) to afford (R)- or (S)-1-(4-((7-(1-methyl-1H-pyrazol-4-yl)imidazo[1,2-c]pyrimidin-5-yl)oxy)azepan-1-yl)prop-2-en-1-one (17 mg, yield: 45%) as a clear oil. ESI-MS (M+H)⁺: 367.2. ¹H NMR (500 MHz, DMSO-d₆) δ 8.25 (d, J=12.82 Hz, 1H), 8.04 (d, J=3.66 Hz, 1H), 7.70 (s, 1H), 7.50 (t, J=1.53 Hz, 1H), 7.44 (s, 1H), 6.82 (ddd, J=8.55, 10.38, 16.48 Hz, 1H), 6.17 (dd, J=2.44, 16.48 Hz, 1H), 5.69 (dt, J=2.44, 10.38 Hz, 1H), 5.50-5.64 (m, 1H), 3.89 (d, J=1.22 Hz, 3H), 3.75-3.84 (m, 1H), 3.59-3.72 (m, 3H), 2.21 (dq, J=3.05, 7.32 Hz, 1H), 2.06-2.17 (m, 2H), 1.87-2.01 (m, 2H), 1.70-1.82 (m, 1H).

### Example 35: (S) or (R)-1-(4-((7-(1-methyl-1H-pyrazol-4-yl)imidazo[1,2-c]pyrimidin-5-yl)oxy)azepan-1-yl)prop-2-yn-1-one

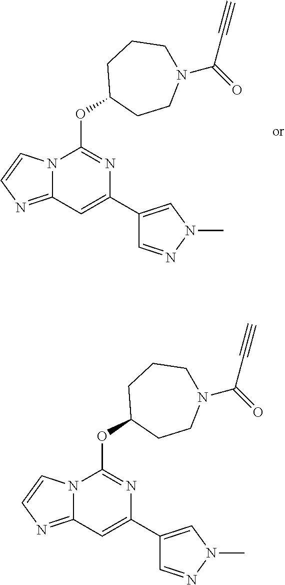 [SMILES: C#CC(=O)N1CCC[C@@H](Oc2nc(-c3cnn(C)c3)cc3nccn23)CC1 ; C#CC(=O)N1CCC[C@H](Oc2nc(-c3cnn(C)c3)cc3nccn23)CC1]

### Synthesis of (R) or (S) 1-(4-((7-(1-methyl-1H-pyrazol-4-yl)imidazo[1,2-c]pyrimidin-5-yl)oxy)azepan-1-yl)prop-2-yn-1-one

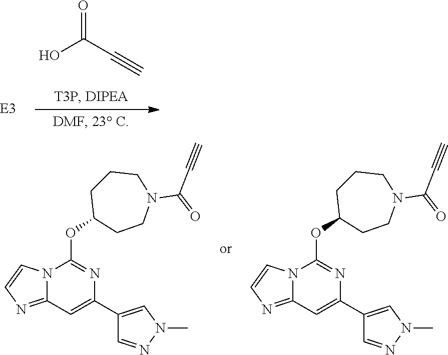

To a solution of E3,5-(4R)- or (4S)-(azepan-4-yloxy)-7-(1-methylpyrazol-4-yl)imidazo[1,2-c]pyrimidine (30 mg, 96 μmol) in DMF (1 mL) was added propiolic acid (13 mg, 192 μmol, 12 μL) followed by DIPEA (25 mg, 192 μmol, 34 μL) with stirring at room temperature. After stirring at room temperature for 5 minutes, T3P (122 mg, 192 μmol, 50% purity) was added dropwise with stirring. After stirring at room temperature for a further 3 hours, the mixture was diluted with ethyl acetate and washed with water. The organic phase was dried with Na₂SO₄, filtered, and concentrated. The residual white solid was re-dissolved in DMSO and purified via reverse phase HPLC (Waters XSelect CSH C18, 5 μm, 19 mm×100 mm column with mobile phase H₂O (A) and MeCN (B) and a gradient of 5-40% B (0.2% NH₄OH final v/v % modifier) with flow rate at 30 mL/min) to afford (R) or (S)-1-(4-((7-(1-methyl-1H-pyrazol-4-yl)imidazo[1,2-c]pyrimidin-5-yl)oxy)azepan-1-yl)prop-2-yn-1-one (2 mg, yield: 5%) as a yellow oil. ESI-MS (M+H)⁺: 365.2. ¹H NMR (500 MHz, DMSO-d₆) δ 8.26 (d, J=2.44 Hz, 1H), 8.05 (d, J=4.27 Hz, 1H), 7.73 (d, J=11.60 Hz, 1H), 7.52 (t, J=1.53 Hz, 1H), 7.46 (d, J=2.44 Hz, 1H), 5.55-5.67 (m, 1H), 4.53 (d, J=17.70 Hz, 1H), 3.89 (s, 3H), 3.78-3.82 (m, 1H), 3.53-3.72 (m, 2H), 3.40-3.52 (m, 1H), 2.21-2.28 (m, 1H), 2.07-2.19 (m, 2H), 1.91-2.06 (m, 2H), 1.72-1.89 (m, 1H).

### Example 36: (R)- or (S)-7-(1-methyl-1H-pyrazol-4-yl)-5-((1-(vinylsulfonyl)azepan-4-yl)oxy)imidazo[1,2-c]pyrimidine

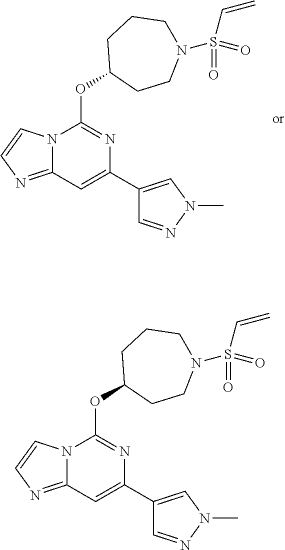 [SMILES: C=CS(=O)(=O)N1CCC[C@@H](Oc2nc(-c3cnn(C)c3)cc3nccn23)CC1 ; C=CS(=O)(=O)N1CCC[C@H](Oc2nc(-c3cnn(C)c3)cc3nccn23)CC1]

### Synthesis of (R)- or (S)-7-(1-methyl-1H-pyrazol-4-yl)-5-((1-(vinylsulfonyl)azepan-4-yl)oxy)imidazo[1,2-c]pyrimidine

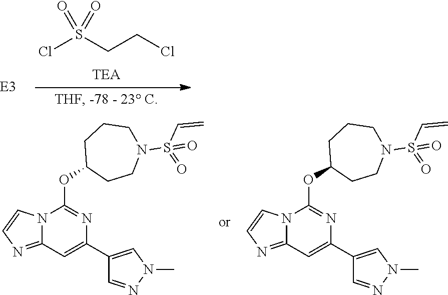 [SMILES: O=S(=O)(Cl)CCCl ; C=CS(=O)(=O)N1CCC[C@@H](Oc2nc(-c3cnn(C)c3)cc3nccn23)CC1 ; C=CS(=O)(=O)N1CCC[C@H](Oc2nc(-c3cnn(C)c3)cc3nccn23)CC1]

To a solution of E3,5-(4R)- or (4S)-(azepan-4-yloxy)-7-(1-methylpyrazol-4-yl)imidazo[1,2-c]pyrimidine (30 mg, 96 μmol) in THF (1 mL) was added 2-chloroethanesulfonyl chloride (31 mg, 192 μmol, 20 μL) upon which precipitation occurred instantaneously. Then, TEA (39 mg, 384 μmol, 53 μL) was added with stirring at room temperature. After stirring overnight, the volatiles were removed and the residual material was re-dissolved in DMSO and purified via reverse phase HPLC (Waters XSelect CSH C18, 5 μm, 19 mm×100 mm column with mobile phase H₂O (A) and MeCN (B) and a gradient of 5-40% B (0.2% NH₄OH final v/v % modifier) with flow rate at 30 mL/min) to afford (R)- or (S)-7-(1-methyl-1H-pyrazol-4-yl)-5-((1-(vinylsulfonyl)azepan-4-yl)oxy)imidazo[1,2-c]pyrimidine (2 mg, yield: 4%) as a yellow solid. ESI-MS (M+H)⁺: 403.2.

### Example 37: (R)- or (S)-1-(4-((7-(1-methyl-1H-pyrazol-4-yl)imidazo[1,2-c]pyrimidin-5-yl)oxy)azepan-1-yl)prop-2-yn-1-one

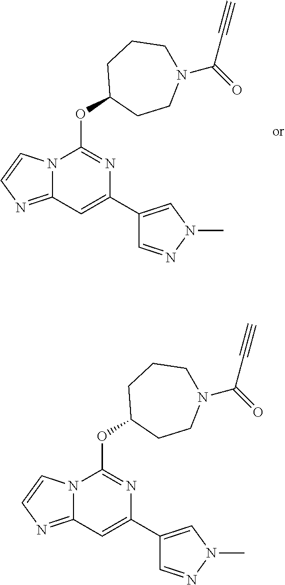 [SMILES: C#CC(=O)N1CCC[C@H](Oc2nc(-c3cnn(C)c3)cc3nccn23)CC1 ; C#CC(=O)N1CCC[C@@H](Oc2nc(-c3cnn(C)c3)cc3nccn23)CC1]

### 1. Synthesis of (R) or (S)-5-(azepan-4-yloxy)-7-(1-methyl-1H-pyrazol-4-yl)imidazo[1,2-c]pyrimidine

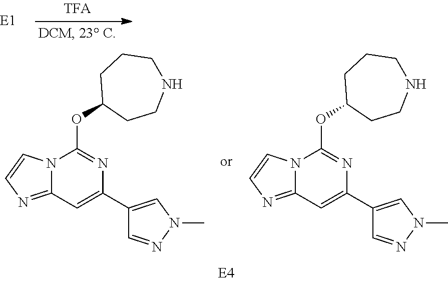 [SMILES: Cn1cc(-c2cc3nccn3c(O[C@H]3CCCNCC3)n2)cn1 ; Cn1cc(-c2cc3nccn3c(O[C@@H]3CCCNCC3)n2)cn1]

To a solution of E1, tert-butyl (4R) or (4S)-4-[7-(1-methylpyrazol-4-yl)imidazo[1,2-c]pyrimidin-5-yl]oxyazepane-1-carboxylate (172 mg, 416 μmol) in DCM (3 mL) was added TFA (949 mg, 8.33 mmol, 637 μL) with stirring at room temperature. After stirring overnight the reaction was dissolved in MeOH and purified on a 10 g SCX column where the desired product was eluted with 2 M NH₃-MeOH to give E4, 5-[(4R) or (4S)-azepan-4-yl]oxy-7-(1-methylpyrazol-4-yl)imidazo[1,2-c]pyrimidine (95 mg, yield: 69%) as a white solid. ESI-MS (M+H)⁺: 313.2.

### 2. Synthesis of (R) or (S)-1-(4-((7-(1-methyl-1H-pyrazol-4-yl)imidazo[1,2-c]pyrimidin-5-yl)oxy)azepan-1-yl)prop-2-yn-1-one

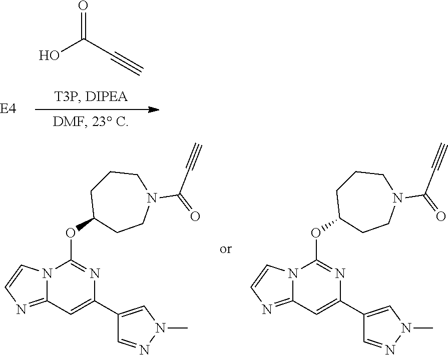

To a solution of E4, 5-[(4R)— or (4S)-azepan-4-yl]oxy-7-(1-methylpyrazol-4-yl)imidazo[1,2-c]pyrimidine (30 mg, 96 μmol) in DMF (1 mL) was added propiolic acid (13 mg, 192 μmol, 12 μL) followed by DIPEA (25 mg, 192 μmol, 34 μL) with stirring at room temperature. After stirring at room temperature for 5 minutes, T3P (122 mg, 192 μmol, 50% purity) was added dropwise with stirring. After stirring at room temperature for a further 3 h, the mixture was diluted with ethyl acetate and washed with water. The organic phase was dried with Na₂SO₄, filtered, and concentrated. The residual white solid was re-dissolved in DMSO and purified via reverse phase HPLC (Waters XSelect CSH C18, 5 μm, 19 mm×100 mm column with mobile phase H₂O (A) and MeCN (B) and a gradient of 5-40% B (0.2% NH₄OH final v/v % modifier) with flow rate at 30 mL/min) to afford (R)- or (S)-1-(4-((7-(1-methyl-1H-pyrazol-4-yl)imidazo[1,2-c]pyrimidin-5-yl)oxy)azepan-1-yl)prop-2-yn-1-one (1 mg, yield: 3%) as a clear oil. ESI-MS (M+H)⁺: 365.2.

### Example 38: (R)- or (S)-1-(4-((7-(1-methyl-1H-pyrazol-4-yl)imidazo[1,2-c]pyrimidin-5-yl)oxy)azepan-1-yl)prop-2-en-1-one

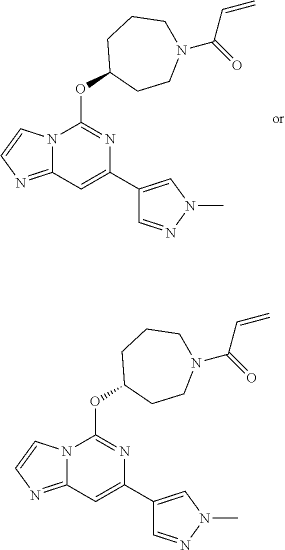 [SMILES: C=CC(=O)N1CCC[C@H](Oc2nc(-c3cnn(C)c3)cc3nccn23)CC1 ; C=CC(=O)N1CCC[C@@H](Oc2nc(-c3cnn(C)c3)cc3nccn23)CC1]

### 1. Synthesis of (R)- or (S)-1-(4-((7-(1-methyl-1H-pyrazol-4-yl)imidazo[1,2-c]pyrimidin-5-yl)oxy)azepan-1-yl)prop-2-en-1-one

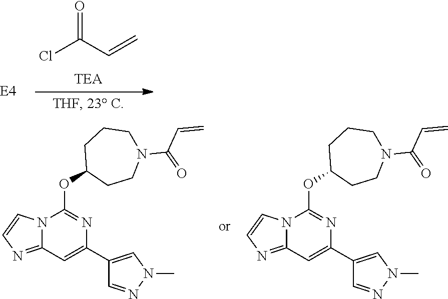

To a solution of E4, 5-[(4R)— or (4S)-azepan-4-yl]oxy-7-(1-methylpyrazol-4-yl)imidazo[1,2-c]pyrimidine (32 mg, 104 μmol) in THF (1 mL) was added prop-2-enoyl chloride (19 mg, 207 μmol, 17 μL) upon which precipitation occurred instantaneously. Then, TEA (21 mg, 207 μmol, 29 μL) was added with stirring at room temperature. After stirring overnight, the volatiles were removed and the residual material was re-dissolved in DMSO and purified via reverse phase HPLC (Waters XSelect CSH C18, 5 m, 19 mm×100 mm column with mobile phase H₂O (A) and MeCN (B) and a gradient of 5-35% B (0.2% NH₄OH final v/v % modifier) with flow rate at 30 mL/min) to afford (R)- or (S)-1-(4-((7-(1-methyl-1H-pyrazol-4-yl)imidazo[1,2-c]pyrimidin-5-yl)oxy)azepan-1-yl)prop-2-en-1-one (11 mg, yield: 27%) as a yellow oil. ESI-MS (M+H)⁺: 367.2. ¹H NMR (500 MHz, DMSO-d₆) δ 8.26 (d, J=12.82 Hz, 1H), 8.04 (d, J=3.05 Hz, 1H), 7.70 (s, 1H), 7.51 (t, J=1.53 Hz, 1H), 7.44 (s, 1H), 6.82 (ddd, J=8.55, 10.38, 16.48 Hz, 1H), 6.17 (dd, J=2.14, 16.18 Hz, 1H), 5.69 (dt, J=2.44, 10.38 Hz, 1H), 5.53-5.64 (m, 1H), 3.89 (s, 3H), 3.75-3.83 (m, 1H), 3.50-3.71 (m, 3H), 2.18-2.26 (m, 1H), 2.06-2.17 (m, 2H), 1.91-2.02 (m, 2H), 1.69-1.82 (m, 1H).

### Example 39: (R)-1-(3-((7-(1-methyl-1H-pyrazol-4-yl)imidazo[1,2-c]pyrimidin-5-yl)oxy)pyrrolidin-1-yl)prop-2-yn-1-one

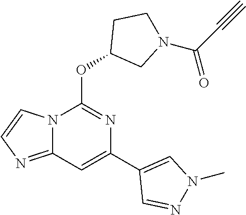 [SMILES: C#CC(=O)N1CC[C@@H](Oc2nc(-c3cnn(C)c3)cc3nccn23)C1]

### Synthesis of tert-butyl (R)-3-((7-(1-methyl-1H-pyrazol-4-yl)imidazo[1,2-c]pyrimidin-5-yl)oxy)pyrrolidine-1-carboxylate

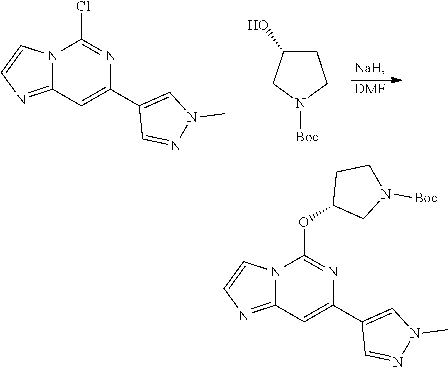

To a vial was added 5-chloro-7-(1-methylpyrazol-4-yl)imidazo[1,2-c]pyrimidine (250 mg, 1.07 mmol), DMF (5.4 mL), sodium hydride (64 mg, 1.61 mmol, 60% suspension in mineral oil) and tert-butyl (3R)-3-hydroxypyrrolidine-1-carboxylate (200 mg, 1.07 mmol). The vial was stirred at 80° C. overnight. The mixture was then diluted with MeOH and concentrated and the residue was then purified by silica gel column chromatography (10-100% [3:1 EtOAc:EtOH] in heptanes) to afford tert-butyl (3R)-3-[7-(1-methylpyrazol-4-yl)imidazo[1,2-c]pyrimidin-5-yl]oxypyrrolidine-1-carboxylate (310 mg, 75% yield) as a mixture with the starting aryl chloride which was used without further purification. LCMS m/z=385.1 (M+H)+.

### Synthesis of (R)-7-(1-methyl-1H-pyrazol-4-yl)-5-(pyrrolidin-3-yloxy)imidazo[1,2-c]pyrimidine hydrochloride

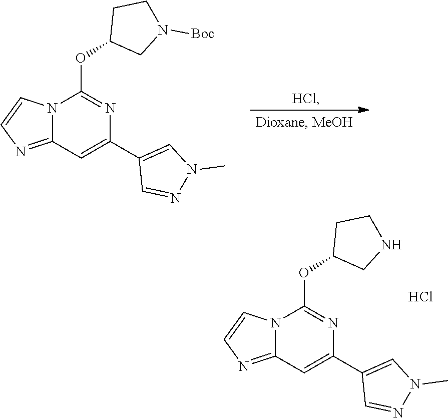 [SMILES: Cn1cc(-c2cc3nccn3c(O[C@@H]3CCN(C(=O)OC(C)(C)C)C3)n2)cn1 ; "HCl," ; Cn1cc(-c2cc3nccn3c(O[C@@H]3CCNC3)n2)cn1 ; Cl]

A solution of tert-butyl (3R)-3-[7-(1-methylpyrazol-4-yl)imidazo[1,2-c]pyrimidin-5-yl]oxypyrrolidine-1-carboxylate (310 mg, 806 μmol) in methanol (4 mL) was treated with HCl (4 M in dioxane, 2.0 mL) and the resulting mixture was stirred at room temperature for 1 hr. The mixture was then concentrated in vacuo and the solid residue was used without further purification. LCMS m/z=285.0 (M+H)+.

### Synthesis of (R)-1-(3-((7-(1-methyl-1H-pyrazol-4-yl)imidazo[1,2-c]pyrimidin-5-yl)oxy)pyrrolidin-1-yl)prop-2-yn-1-one

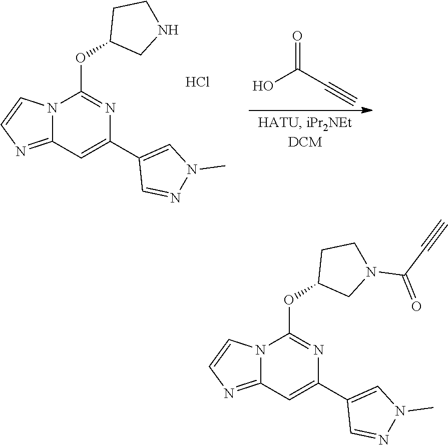

To a vial was added 7-(1-methylpyrazol-4-yl)-5-[(3R)-pyrrolidin-3-yl]oxy-imidazo[1,2-c]pyrimidine hydrochloride (114 mg, 355 μmol), DCM (3.6 mL), N-ethyl-N-isopropyl-propan-2-amine (310 μL, 1.78 mmol) then prop-2-ynoic acid (33 μL, 533 μmol). The vial was stirred at room temperature for 16 hours. The mixture was then concentrated and the residue was purified via silica gel column chromatography (10-100% [13:1 EtOAc:EtOH] in heptanes). Fractions were combined and concentrated then purified by prep HPLC (Waters SunFire Prep, C18 5 μm, OBD 30×50 mm, eluting with 10-70% MeCN:H₂O [with 0.1% TFA modifier]) to afford 1-[(3R)-3-[7-(1-methylpyrazol-4-yl)imidazo[1,2-c]pyrimidin-5-yl]oxypyrrolidin-1-yl]prop-2-yn-1-one (27.3 mg, 23% yield) as a solid. LCMS m/z=337.0 (M+H)+. ¹H NMR (500 MHz, MeOD-d₄) δ: 8.46 (d, J=4.3 Hz, 1H), 8.23 (d, J=2.4 Hz, 1H), 8.12-8.07 (m, 1H), 7.92 (d, J=2.4 Hz, 1H), 7.65 (d, J=4.9 Hz, 1H), 6.21-6.09 (m, 1H), 4.33-4.22 (m, 1H), 4.16-4.07 (m, 1H), 4.01 (d, J=1.2 Hz, 5H), 3.92-3.68 (m, 1H), 2.66-2.47 (in, 2H).

### Examples 40 to 53

The compounds in the following table were prepared from 5-chloro-7-(1-methylpyrazol-4-yl)imidazo[1,2-c]pyrimidine and the appropriate alcohol and carboxylic acid, following the steps described in Example 39:

| Ex No | Structure, Starting Material, Name | Yield, Data |
|---|---|---|
| 40 | 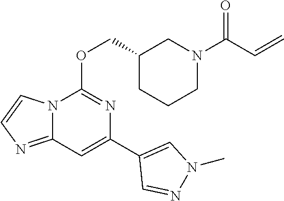 [SMILES: C=CC(=O)N1CCC[C@H](COc2nc(-c3cnn(C)c3)cc3nccn23)C1] | 34.3 mg, 29% yield LCMS m/z = 367.2 (M + H)+ |
| | SM: acryloyl chloride and tert-butyl (3S)- | |
| | 3-(hydroxymethyl)piperidine-1- | |
| | carboxylate | |
| 41 | 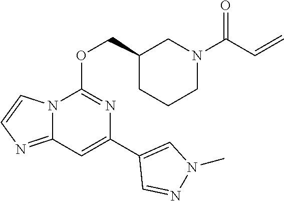 [SMILES: C=CC(=O)N1CCC[C@@H](COc2nc(-c3cnn(C)c3)cc3nccn23)C1] | 23.0 mg, 22% yield LCMS m/z = 367.2 (M + H)+ |
| | SM: acryloyl chloride and tert-butyl (3R)- | |
| | 3-(hydroxymethyl)piperidine-1- | |
| | carboxylate | |
| 42 | 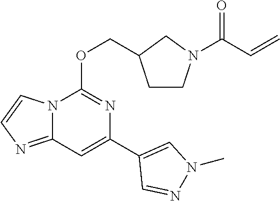 [SMILES: C=CC(=O)N1CCC(COc2nc(-c3cnn(C)c3)cc3nccn23)C1] | 13.2 mg, 11% yield LCMS m/z = 353.2 (M + H)+ |
| | SM: acryloyl chloride and tert-butyl 3- | |
| | (hydroxymethyl)pyrrolidine-1-carboxylate | |
| 43 | 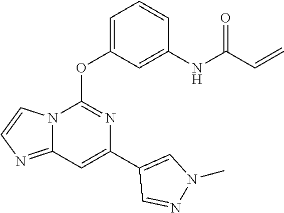 [SMILES: C=CC(=O)Nc1cccc(Oc2nc(-c3cnn(C)c3)cc3nccn23)c1] | 30.1 mg, 26% yield LCMS m/z = 361.2 (M + H)+ |
| | SM: acryloyl chloride and tert-butyl (3- | |
| | hydroxyphenyl)carbamate | |
| 44 | 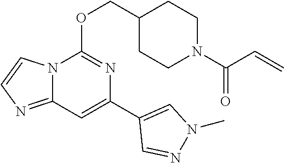 [SMILES: C=CC(=O)N1CCC(COc2nc(-c3cnn(C)c3)cc3nccn23)CC1] | 5.5 mg, 6% yield. LCMS m/z = 367.2 (M + H)+ |
| | SM: acryloyl chloride and tert-butyl 4- | |
| | (hydroxymethyl)piperidine-1-carboxylate | |
| 45 | 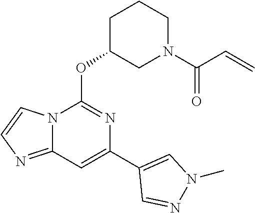 [SMILES: C=CC(=O)N1CCC[C@@H](Oc2nc(-c3cnn(C)c3)cc3nccn23)C1] | 50.9 mg, 43% yield. LCMS m/z = 353.2 (M + H)+ |
| | SM: acryloyl chloride and tert-butyl (R)-3- | |
| | hydroxypiperidine-1-carboxylate | |
| 46 | 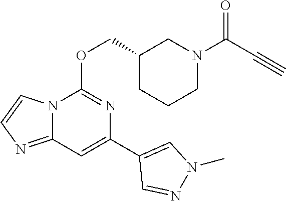 [SMILES: C#CC(=O)N1CCC[C@H](COc2nc(-c3cnn(C)c3)cc3nccn23)C1] | 9.2 mg, 9% yield LCMS m/z = 365.2 (M + H)+ |
| | SM: propiolic acid and tert-butyl (S)-3- | |
| | (hydroxymethyl)piperidine-1-carboxylate | |
| 47 | 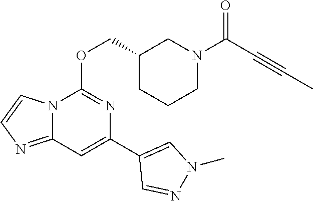 [SMILES: CC#CC(=O)N1CCC[C@H](COc2nc(-c3cnn(C)c3)cc3nccn23)C1] | 7.3 mg, 10% yield LCMS m/z = 379.2 (M + H)+ |
| | SM: acryloyl chloride but-2-ynoic acid and | |
| | tert-butyl (S)-3- | |
| | (hydroxymethyl)piperidine-1-carboxylate | |
| 48 | 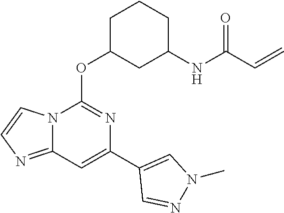 [SMILES: C=CC(=O)NC1CCCC(Oc2nc(-c3cnn(C)c3)cc3nccn23)C1] | 21.1 mg, 15% yield. LCMS m/z = 367.2 (M + H)+ |
| | SM: acryloyl chloride and tert-butyl (3- | |
| | hydroxycyclohexyl)carbamate | |
| 49 | 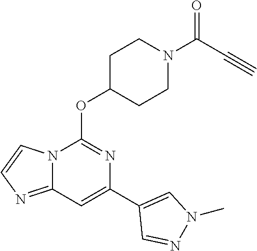 [SMILES: C#CC(=O)N1CCC(Oc2nc(-c3cnn(C)c3)cc3nccn23)CC1] | 12.9 mg, 12% yield LCMS m/z = 351.2 (M + H)+ |
| | SM: propiolic acid and tert-butyl 4- | |
| | hydroxypiperidine-1-carboxylate | |
| 50 | 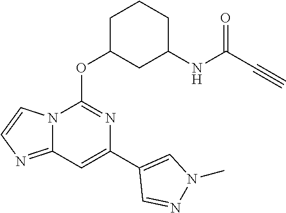 [SMILES: C#CC(=O)NC1CCCC(Oc2nc(-c3cnn(C)c3)cc3nccn23)C1] | 7.1 mg, 6% yield LCMS m/z = 364.2 (M + H)+ |
| | SM: propiolic acid and tert-butyl (3- | |
| | hydroxycyclohexyl)carbamate | |
| 51 | 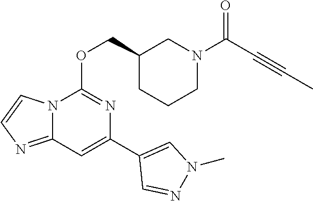 [SMILES: CC#CC(=O)N1CCC[C@@H](COc2nc(-c3cnn(C)c3)cc3nccn23)C1] | 24.2 mg, 34% yield LCMS m/z = 379.0 (M + H)+ |
| | SM: but-2-ynoic acid and tert-butyl (R)-3- | |
| | (hydroxymethyl)piperidine-1-carboxylate | |
| 52 | 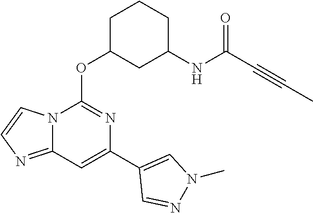 [SMILES: CC#CC(=O)NC1CCCC(Oc2nc(-c3cnn(C)c3)cc3nccn23)C1] | 10.4 mg, 28% yield LCMS m/z = 379.2 (M + H)+ |
| | SM: but-2-ynoic acid and tert-butyl (3- | |
| | hydroxycyclohexyl)carbamate | |
| 53 | 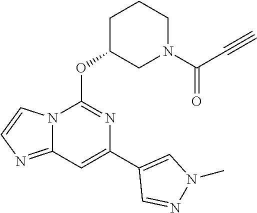 [SMILES: C#CC(=O)N1CCC[C@@H](Oc2nc(-c3cnn(C)c3)cc3nccn23)C1] | 8.9 mg, 8% yield LCMS m/z = 351.4 (M + H)+ |
| | SM: propiolic acid and tert-butyl (R)-3- | |
| | hydroxypiperidine-1-carboxylate | |

### Example 54: (S)-1-(6-((6-(1-methyl-1H-pyrazol-4-yl)pyrazolo[1,5-a]pyrazin-4-yl)oxy)-1,4-oxazepan-4-yl)prop-2-en-1-one

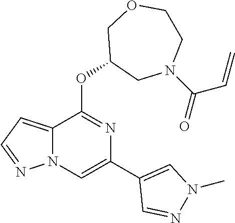 [SMILES: C=CC(=O)N1CCOC[C@@H](Oc2nc(-c3cnn(C)c3)cn3nccc23)C1]

### Synthesis of tert-butyl (6S)-6-[6-(1-methylpyrazol-4-yl)pyrazolo[1,5-a]pyrazin-4-yl]oxy-1,4-oxazepane-4-carboxylate

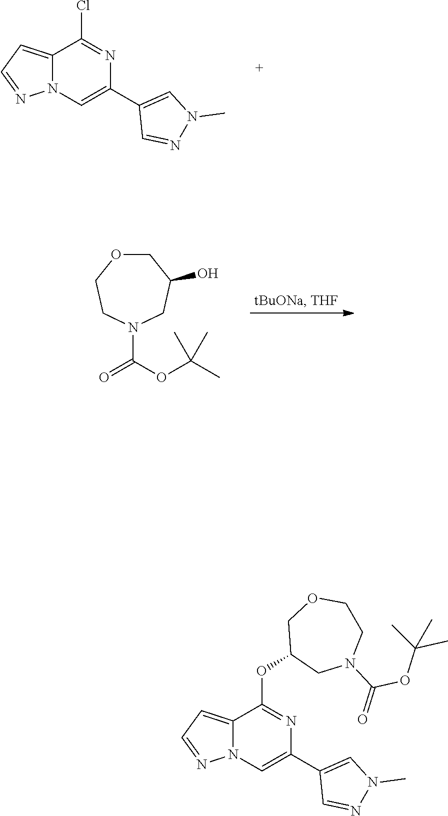 [SMILES: Cn1cc(-c2cn3nccc3c(Cl)n2)cn1 ; CC(C)(C)OC(=O)N1CCOC[C@@H](O)C1 ; Cn1cc(-c2cn3nccc3c(O[C@@H]3COCCN(C(=O)OC(C)(C)C)C3)n2)cn1]

A flask containing tert-butyl (6S)-6-hydroxy-1,4-oxazepane-4-carboxylate (247 mg, 1.14 mmol) in anhydrous THF (2 mL) was cooled in an ice water bath, then sodium tert-butoxide (168 mg, 1.74 mmol) was added carefully in portions to the cold mixture. After 10 minutes, 4-chloro-6-(1-methylpyrazol-4-yl)pyrazolo[1,5-a]pyrazine (235 mg, 1.01 mmol) was added carefully in portions to the cold heterogeneous mixture. Upon complete addition of 4-chloro-6-(1-methylpyrazol-4-yl)pyrazolo[1,5-a]pyrazine, the mixture was allowed to warm to 23° C. and monitored with LCMS. After 18 hours, the reaction was carefully quenched with slow addition of water, then the biphasic mixture was extracted three times with ethyl acetate. The organics were pooled then dried over anhydrous sodium sulfate. After filtration and concentration under reduced pressure, the residue was loaded onto a silica gel column and purified with (30-100% ethyl acetate in heptane.) The desired fractions were pooled then concentrated under reduced pressure to afford a white solid as tert-butyl (6S)-6-[6-(1-methylpyrazol-4-yl)pyrazolo[1,5-a]pyrazin-4-yl]oxy-1,4-oxazepane-4-carboxylate (319 mg, 76% yield) that was used without further purification. LCMS m/z=415.1 (M+H)⁺. ¹H NMR (500 MHz, DMSO-d₆) d=8.77 (s, 1H), 8.29-8.17 (m, 1H), 8.11-7.91 (m, 2H), 6.85-6.75 (m, 1H), 5.62-5.53 (m, 1H), 4.28-3.94 (m, 3H), 3.93-3.74 (m, 4H), 3.47-3.36 (m, 1H), 3.73-3.32 (m, 3H), 1.76-0.98 (m, 9H).

### Synthesis of (6S)-6-[6-(1-methylpyrazol-4-yl)pyrazolo[1,5-a]pyrazin-4-yl]oxy-1,4-oxazepane

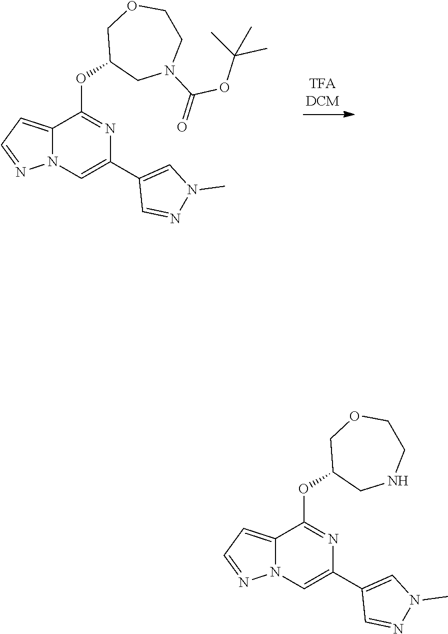 [SMILES: Cn1cc(-c2cn3nccc3c(O[C@@H]3COCCN(C(=O)OC(C)(C)C)C3)n2)cn1 ; Cn1cc(-c2cn3nccc3c(O[C@H]3CNCCOC3)n2)cn1]

A vial containing tert-butyl (6S)-6-[6-(1-methylpyrazol-4-yl)pyrazolo[1,5-a]pyrazin-4-yl]oxy-1,4-oxazepane-4-carboxylate (319 mg, 769 μmol) in anhydrous dichloromethane (2 mL) was cooled in an ice water bath, then trifluoroacetic acid (1 mL, 13 mmol) was added carefully dropwise to the cold mixture. Upon complete addition of TFA, the mixture was allowed to warm to 23° C. and monitored with LCMS. After 1 hour, the reaction was carefully concentrated under reduced pressure to afford a light yellow film as (6S)-6-[6-(1-methylpyrazol-4-yl)pyrazolo[1,5-a]pyrazin-4-yl]oxy-1,4-oxazepane (345 mg, TFA salt) that was used without further purification. LCMS m/z=315.0 (M+H)⁺.

### Synthesis of 1-[(6S)-6-[6-(1-methylpyrazol-4-yl)pyrazolo[1,5-a]pyrazin-4-yl]oxy-1,4-oxazepan-4-yl]prop-2-en-1-one

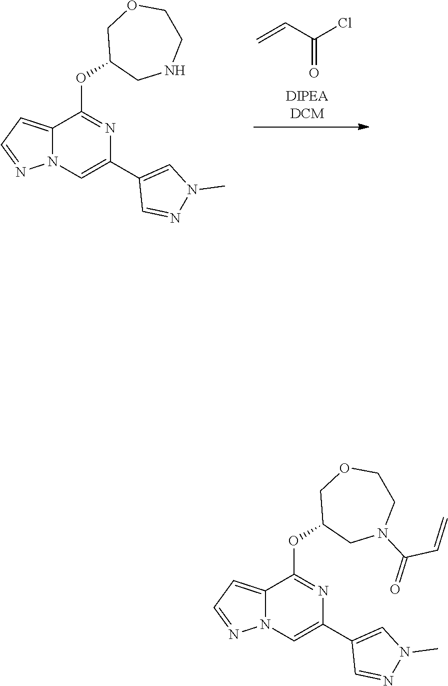

To a vial containing (6S)-6-[6-(1-methylpyrazol-4-yl)pyrazolo[1,5-a]pyrazin-4-yl]oxy-1,4-oxazepane (329 mg, 769 μmol, TFA salt) in anhydrous dichloromethane (3 mL) was added Hunigs base (0.7 mL, 4.02 mmol) carefully dropwise at −25° C. After 5 minutes, acryloyl chloride (0.2 mL, 2.46 mmol) was added carefully dropwise to the cold homogeneous solution. Upon complete addition of acryloyl chloride, the reaction was allowed to warm to 23° C. and monitored with LCMS and TLC. After 3 minutes, the reaction was carefully quenched with slow addition of saturated aqueous sodium bicarbonate solution. The biphasic mixture was loaded onto a silica gel column and purified with (15-75% [3:1 ethyl acetate: ethanol] in heptane.) The desired fractions were pooled then concentrated under reduced pressure to afford a white solid as 1-[(6S)-6-[6-(1-methylpyrazol-4-yl)pyrazolo[1,5-a]pyrazin-4-yl]oxy-1,4-oxazepan-4-yl]prop-2-en-1-one (226 mg, 76% yield). ¹H NMR (500 MHz, DMSO-d₆) d=8.82-8.74 (m, 1H), 8.27-8.19 (m, 1H), 8.13-7.96 (m, 2H), 6.92-6.73 (m, 2H), 6.19 (dd, J=2.4, 16.5 Hz, 1H), 5.76-5.60 (m, 2H), 4.53-4.05 (m, 3H), 4.03-3.84 (m, 5H), 3.76-3.50 (m, 3H). LCMS m/z=369.1 (M+H)⁺.

### Example 55: (S)-1-(3-((6-(1-methyl-1H-pyrazol-4-yl)pyrazolo[1,5-a]pyrazin-4-yl)oxy)azepan-1-yl)prop-2-en-1-one

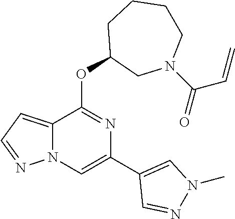 [SMILES: C=CC(=O)N1CCCC[C@H](Oc2nc(-c3cnn(C)c3)cn3nccc23)C1]

### Synthesis of tert-butyl (3S)-3-[6-(1-methylpyrazol-4-yl)pyrazolo[1,5-a]pyrazin-4-yl]oxyazepane-1-carboxylate

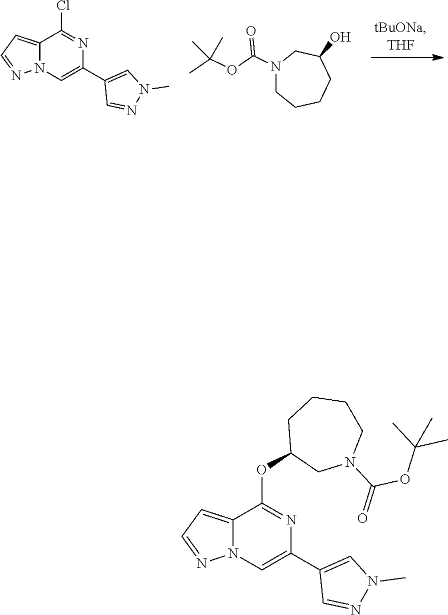

A vial containing tert-butyl (3S)-3-hydroxyazepane-1-carboxylate (464 mg, 2.16 mmol) in anhydrous THF (8 mL) was cooled in an ice water bath, then sodium tert-butoxide (314 mg, 3.27 mmol) was added carefully in portions to the cold mixture. After 15 minutes, 4-chloro-6-(1-methylpyrazol-4-yl)pyrazolo[1,5-a]pyrazine (495 mg, 2.12 mmol) was added carefully in portions to the cold heterogeneous mixture. Upon complete addition of 4-chloro-6-(1-methylpyrazol-4-yl)pyrazolo[1,5-a]pyrazine, the mixture was allowed to warm to 23° C. and monitored with LCMS. After 2.5 hours, the reaction was carefully quenched with slow addition of water. The biphasic mixture was extracted three times with ethyl acetate then dried over anhydrous sodium sulfate. After filtration and concentration under reduced pressure, the residue was loaded onto a silica gel column and purified with (0-30% [3:1 ethyl acetate: ethanol] in heptane.) The desired fractions were pooled then concentrated under reduced pressure to afford a yellow film as tert-butyl (3S)-3-[6-(1-methylpyrazol-4-yl)pyrazolo[1,5-a]pyrazin-4-yl]oxyazepane-1-carboxylate that was used without further purification. LCMS m/z=413.2 (M+H)⁺.

### Synthesis of 4-[(3S)-azepan-3-yl]oxy-6-(1-methylpyrazol-4-yl)pyrazolo[1,5-a]pyrazine

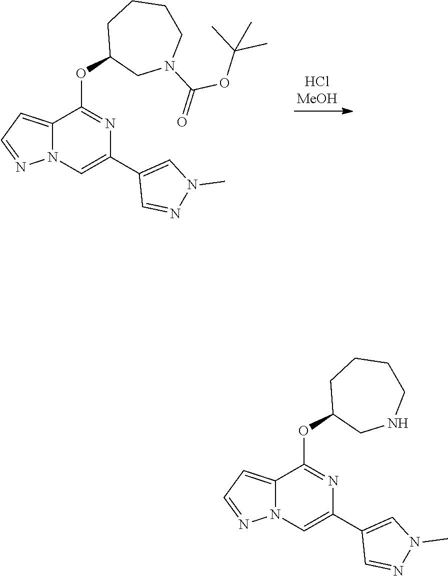 [SMILES: Cn1cc(-c2cn3nccc3c(O[C@H]3CCCCN(C(=O)OC(C)(C)C)C3)n2)cn1 ; Cn1cc(-c2cn3nccc3c(O[C@H]3CCCCNC3)n2)cn1]

A vial containing tert-butyl (3S)-3-[6-(1-methylpyrazol-4-yl)pyrazolo[1,5-a]pyrazin-4-yl]oxyazepane-1-carboxylate (787 mg, 1.91 mmol) in anhydrous methanol (2 mL) was cooled in an ice water bath, then HCl (1.25 M in methanol, 4 mL) was added carefully dropwise to the cold mixture. Upon complete addition of 1.25 M HCl in methanol, the mixture was allowed to warm to 23° C. and monitored with LCMS. After 6 days, the reaction was carefully concentrated under reduced pressure to afford a light yellow film as 4-[(3S)-azepan-3-yl]oxy-6-(1-methylpyrazol-4-yl)pyrazolo[1,5-a]pyrazine hydrochloride (672 mg, crude) that was used without further purification. LCMS: m/z=313.2 (M+H)⁺.

### Synthesis of 1-[(3S)-3-[6-(1-methylpyrazol-4-yl)pyrazolo[1,5-a]pyrazin-4-yl]oxyazepan-1-yl]prop-2-en-1-one

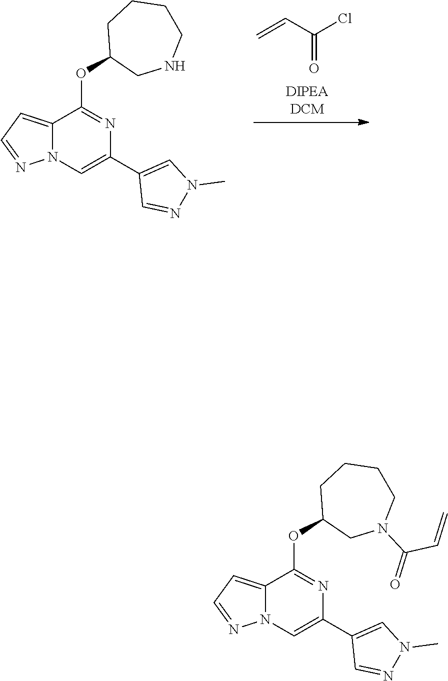

To a vial containing 4-[(3S)-azepan-3-yl]oxy-6-(1-methylpyrazol-4-yl)pyrazolo[1,5-a]pyrazine hydrochloride (288 mg, 824 μmol) in anhydrous dichloromethane (2 mL) was added Hunigs base (0.7 mL, 4.02 mmol) carefully dropwise at −25° C. After 5 minutes, acryloyl chloride (0.15 mL, 1.85 mmol) was added carefully dropwise to the cold homogeneous solution. Upon complete addition of acryloyl chloride, the reaction was allowed to warm to 23° C. and monitored with LCMS and TLC. After 3 minutes, the reaction was carefully quenched with slow addition of saturated aqueous sodium bicarbonate solution. The biphasic mixture was loaded onto a silica gel column and purified with (15-75% [3:1 ethyl acetate: ethanol] in heptane.) The desired fractions were pooled then concentrated under reduced pressure to afford a white solid as 1-[(3S)-3-[6-(1-methylpyrazol-4-yl)pyrazolo[1,5-a]pyrazin-4-yl]oxyazepan-1-yl]prop-2-en-1-one (73 mg, 23% yield). ¹H NMR (500 MHz, DMSO-d₆) d=8.76 (d, J=6.1 Hz, 1H), 8.29-8.18 (m, 1H), 8.06-7.99 (m, 1H), 6.88-6.68 (m, 2H), 6.16 (dt, J=2.4, 16.8 Hz, 1H), 5.74-5.51 (m, 2H), 4.33 (br dd, J=5.5, 13.4 Hz, 1H), 4.01-3.91 (m, 1H), 3.90-3.64 (m, 3H), 3.64-3.43 (m, 2H), 2.13-2.00 (m, 1H), 2.00-1.62 (m, 4H), 1.62-1.37 (m, 2H). LCMS m/z=367.1 (M+H)⁺.

### Example 56: (R)-1-(3-((6-(1-methyl-1H-pyrazol-4-yl)pyrazolo[1,5-a]pyrazin-4-yl)oxy)azepan-1-yl)prop-2-en-1-one

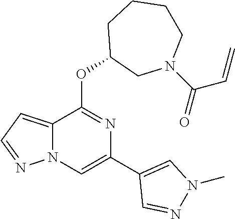 [SMILES: C=CC(=O)N1CCCC[C@@H](Oc2nc(-c3cnn(C)c3)cn3nccc23)C1]

### Synthesis of tert-butyl (3R)-3-[6-(1-methylpyrazol-4-yl)pyrazolo[1,5-a]pyrazin-4-yl]oxyazepane-1-carboxylate

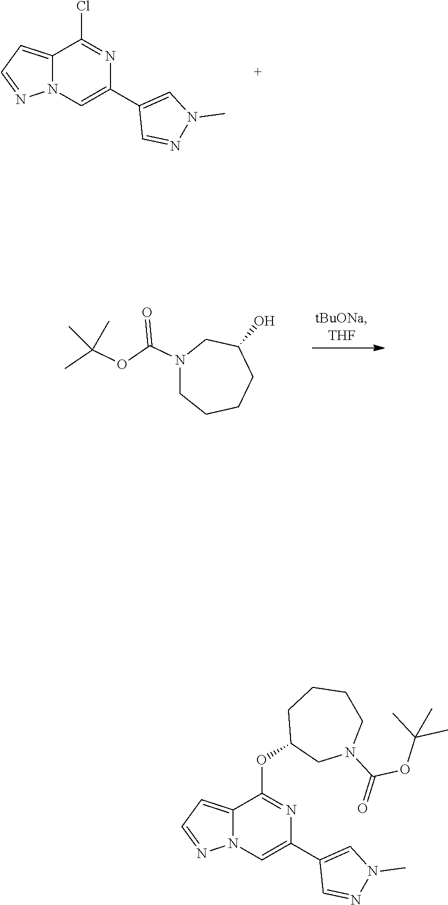 [SMILES: Cn1cc(-c2cn3nccc3c(Cl)n2)cn1 ; CC(C)(C)OC(=O)N1CCCC[C@@H](O)C1 ; Cn1cc(-c2cn3nccc3c(O[C@@H]3CCCCN(C(=O)OC(C)(C)C)C3)n2)cn1]

A vial containing tert-butyl (3R)-3-hydroxyazepane-1-carboxylate (550 mg, 2.56 mmol) in anhydrous THF (9 mL) was cooled in an ice water bath, then sodium tert-butoxide (338 mg, 3.52 mmol) was added carefully in portions to the cold mixture. After 15 minutes, 4-chloro-6-(1-methylpyrazol-4-yl)pyrazolo[1,5-a]pyrazine (537 mg, 2.30 mmol) was added carefully in portions to the cold heterogeneous mixture. Upon complete addition of 4-chloro-6-(1-methylpyrazol-4-yl)pyrazolo[1,5-a]pyrazine, the mixture was allowed to warm to 23° C. and monitored with LCMS. After 2 hours, the reaction was carefully quenched with slow addition of water. The biphasic mixture was extracted three times with ethyl acetate then dried over anhydrous sodium sulfate. After filtration and concentration under reduced pressure, the residue was loaded onto a silica gel column and purified with (20-65% ethyl acetate in heptanes). The desired fractions were pooled then concentrated under reduced pressure to afford a yellow film as tert-butyl (3R)-3-[6-(1-methylpyrazol-4-yl)pyrazolo[1,5-a]pyrazin-4-yl]oxyazepane-1-carboxylate (905.5 mg, 96% yield) that was used without further purification. LCMS: m/z=413.2 (M+H)⁺. ¹H NMR (500 MHz, DMSO-d₆) d=8.79-8.72 (m, 1H), 8.28-8.18 (m, 1H), 8.11-7.97 (m, 2H), 6.78 (dd, J=1.2, 18.3 Hz, 1H), 5.58-5.47 (m, 1H), 4.09-4.02 (m, 1H), 4.01-3.87 (m, 3H), 3.61-3.19 (m, 4H), 1.97-1.78 (m, 3H), 1.78-1.62 (m, 2H), 1.41 (s, 3H), 1.46-1.05 (m, 6H).

### Synthesis of 4-[(3R)-azepan-3-yl]oxy-6-(1-methylpyrazol-4-yl)pyrazolo[1,5-a]pyrazine

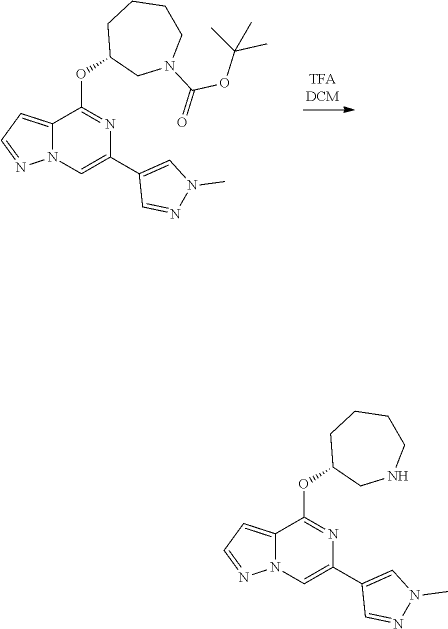 [SMILES: Cn1cc(-c2cn3nccc3c(O[C@@H]3CCCCN(C(=O)OC(C)(C)C)C3)n2)cn1 ; Cn1cc(-c2cn3nccc3c(O[C@@H]3CCCCNC3)n2)cn1]

A vial containing tert-butyl (3R)-3-[6-(1-methylpyrazol-4-yl)pyrazolo[1,5-a]pyrazin-4-yl]oxyazepane-1-carboxylate (905.5 mg, 2.20 mmol) in anhydrous dichloromethane (2 mL) was cooled in an ice water bath, then trifluoroacetic acid (2 mL, 26.1 mmol) was added carefully dropwise to the cold mixture. Upon complete addition of TFA, the mixture was allowed to warm to 23° C. and monitored with LCMS. After 1 hour, the reaction was carefully concentrated under reduced pressure to afford a light yellow film as 4-[(3R)-azepan-3-yl]oxy-6-(1-methylpyrazol-4-yl)pyrazolo[1,5-a]pyrazine (941.1 mg, 100% yield, TFA salt) that was used without further purification. LCMS m/z=313.1 (M+H)⁺.

### Synthesis of 1-[(3R)-3-[6-(1-methylpyrazol-4-yl)pyrazolo[1,5-a]pyrazin-4-yl]oxyazepan-1-yl]prop-2-en-1-one

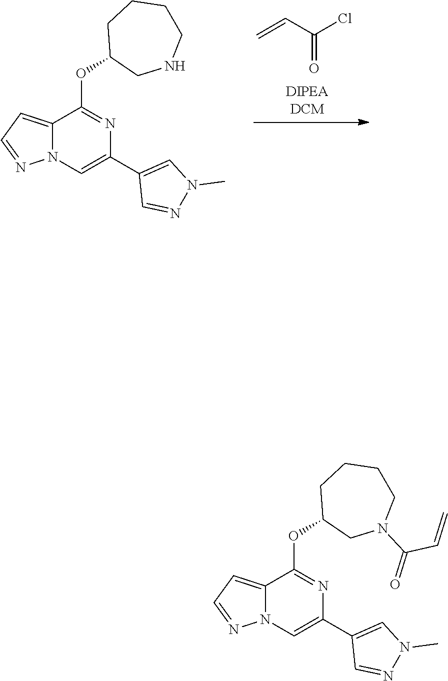

To a vial containing 4-[(3R)-azepan-3-yl]oxy-6-(1-methylpyrazol-4-yl)pyrazolo[1,5-a]pyrazine (191 mg, 612 mmol, TFA salt) in anhydrous dichloromethane (2 mL) was added Hunigs base (0.5 mL, 2.87 mmol) carefully dropwise at −25° C. After 5 minutes, acryloyl chloride (0.1 mL, 1.23 mmol) was added carefully dropwise to the cold homogeneous solution. Upon complete addition of acryloyl chloride, the reaction was allowed to warm to 23° C. and monitored with LCMS and TLC. After 3 minutes, the reaction was carefully quenched with slow addition of saturated aqueous sodium bicarbonate solution. The biphasic mixture was loaded onto a silica gel column and purified with (15-75% [3:1 ethyl acetate ethanol] in heptanes). The desired fractions were pooled then concentrated under reduced pressure to afford a white solid as 1-[(3R)-3-[6-(1-methylpyrazol-4-yl)pyrazolo[1,5-a]pyrazin-4-yl]oxyazepan-1-yl]prop-2-en-1-one. ¹H NMR (500 MHz, DMSO-d₆) d=8.76 (d, J=5.5 Hz, 1H), 8.29-8.18 (m, 1H), 8.06-8.01 (m, 1H), 6.88-6.71 (m, 2H), 6.16 (dt, J=2.1, 16.9 Hz, 1H), 5.74-5.51 (m, 2H), 4.33 (br dd, J=5.5, 14.0 Hz, 1H), 3.99-3.91 (m, 1H), 3.90-3.68 (m, 3H), 3.59-3.43 (m, 2H), 2.06 (ddd, J=4.3, 8.9, 13.7 Hz, 1H), 1.98-1.66 (m, 4H), 1.65-1.42 (m, 2H). LCMS m/z=367.1 (M+H)⁺.

### Example 57: 1-(4-((6-(1-methyl-1H-pyrazol-4-yl)-[1,2,4]triazolo[1,5-a]pyrazin-8-yl)oxy)azepan-1-yl)prop-2-en-1-one

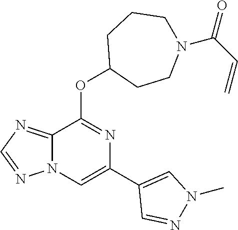 [SMILES: C=CC(=O)N1CCCC(Oc2nc(-c3cnn(C)c3)cn3ncnc23)CC1]

### Synthesis of tert-butyl 4-((6-bromo-[1,2,4]triazolo[1,5-a]pyrazin-8-yl)oxy)azepane-1-carboxylate

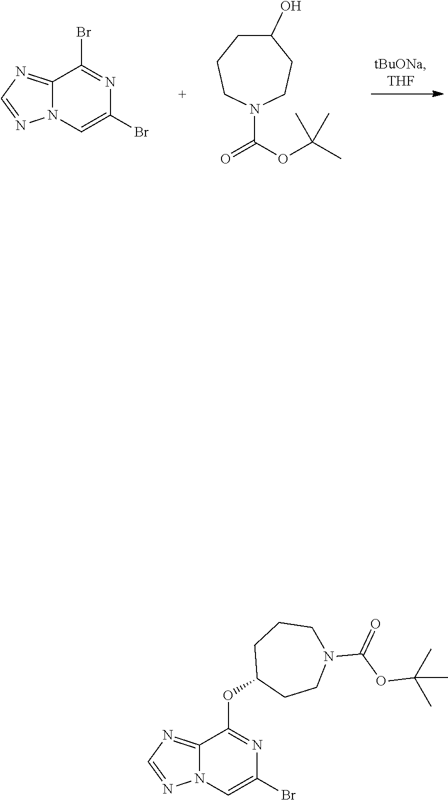 [SMILES: Brc1cn2ncnc2c(Br)n1 ; CC(C)(C)OC(=O)N1CCCC(O)CC1 ; CC(C)(C)OC(=O)N1CCC[C@@H](Oc2nc(Br)cn3ncnc23)CC1]

A flask containing tert-butyl 4-hydroxyazepane-1-carboxylate (659 mg, 3.06 mmol) in anhydrous THF (10 mL) was cooled in an ice water bath, then sodium tert-butoxide (454 mg, 4.72 mmol) was added carefully in portions to the cold mixture. After 15 minutes, 6,8-dibromo-[1,2,4]triazolo[1,5-a]pyrazine (850 mg, 3.06 mmol) was added carefully in portions to the cold heterogeneous mixture. Upon complete addition of 6,8-dibromo-[1,2,4]triazolo[1,5-a]pyrazine, the mixture was allowed to warm to 23° C. and monitored with LCMS. After 2.5 hours, the reaction was carefully quenched with slow addition of water. The biphasic mixture was extracted three times with ethyl acetate then dried over anhydrous sodium sulfate. After filtration and concentration under reduced pressure, the residue was loaded onto a silica gel column and purified with (20-45% ethyl acetate in heptanes). The desired fractions were pooled, then concentrated under reduced pressure to afford a faint yellow film as tert-butyl 4-((6-bromo-[1,2,4]triazolo[1,5-a]pyrazin-8-yl)oxy)azepane-1-carboxylate (952.3 mg, 76% yield) that was used without further purification. LCMS: m/z=412.0 (M+H)⁺. ¹H NMR (500 MHz, DMSO-d₆) d=9.03 (s, 1H), 8.60 (s, 1H), 5.35-5.29 (m, 1H), 3.48-3.39 (m, 3H), 3.34-3.25 (m, 1H), 2.24-2.10 (m, 1H), 1.98-1.83 (m, 4H), 1.68 (br dd, J=4.9, 9.2 Hz, 1H), 1.42 (s, 9H).

### Synthesis of tert-butyl 4-((6-(1-methyl-1H-pyrazol-4-yl)-[1,2,4]triazolo[1,5-a]pyrazin-8-yl)oxy)azepane-1-carboxylate

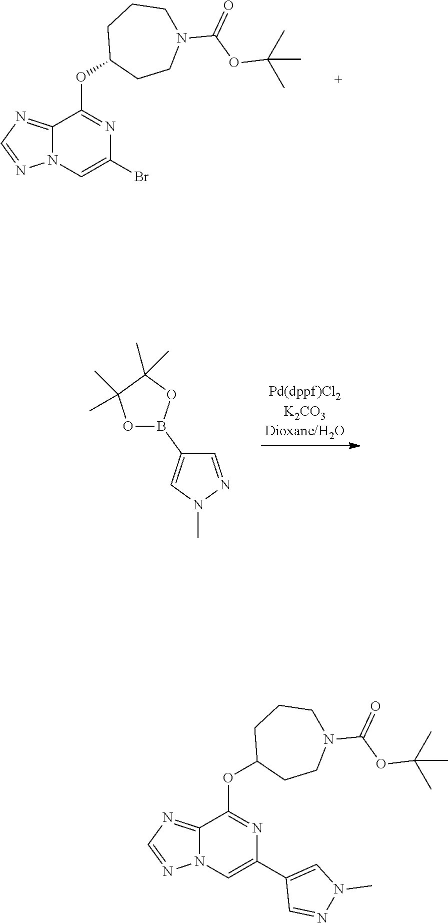

A flask containing tert-butyl 4-((6-bromo-[1,2,4]triazolo[1,5-a]pyrazin-8-yl)oxy)azepane-1-carboxylate (952 mg, 2.31 mmol),1-methyl-4-(4,4,5,5-tetramethyl-1,3,2-dioxaborolan-2-yl)pyrazole (971 mg, 4.67 mmol), Pd(dppf)Cl₂ dichloromethane adduct (300 mg, 367 μmol), and potassium carbonate (961 mg, 6.95 mmol) was degassed then backfilled with nitrogen. Dioxane (6 mL) then water (0.6 mL) were added to the mixture. Upon complete addition of water, the reaction was heated to 90° C. and monitored with LCMS. After 2.5 hours, the reaction was carefully quenched with slow addition of water. The biphasic mixture was extracted three times with ethyl acetate then dried over anhydrous sodium sulfate. After filtration and concentration under reduced pressure, the residue was loaded onto a silica gel column and purified with (25-100% ethyl acetate in heptanes). The desired fractions were pooled then concentrated under reduced pressure to afford a sticky yellow foam as tert-butyl 4-((6-(1-methyl-1H-pyrazol-4-yl)-[1,2,4]triazolo[1,5-a]pyrazin-8-yl)oxy)azepane-1-carboxylate (707.6 mg, 74% yield) that was used without further purification. ¹H NMR (500 MHz, DMSO-d₆) d=8.97 (s, 1H), 8.55 (s, 1H), 8.25 (br d, J=15.3 Hz, 1H), 8.04 (d, J=9.8 Hz, 1H), 5.52-5.45 (m, 1H), 3.59-3.42 (m, 3H), 3.41-3.35 (m, 1H), 3.35-3.25 (m, 4H), 2.26-2.19 (m, 1H), 2.05-1.85 (m, 4H), 1.73 (br dd, J=4.6, 8.9 Hz, 2H), 1.43 (d, J=6.7 Hz, 7H). LCMS m/z=414.2 (M+H)⁺.

### Synthesis of 8-(azepan-4-yloxy)-6-(1-methyl-1H-pyrazol-4-yl)-[1,2,4]triazolo[1,5-a]pyrazine

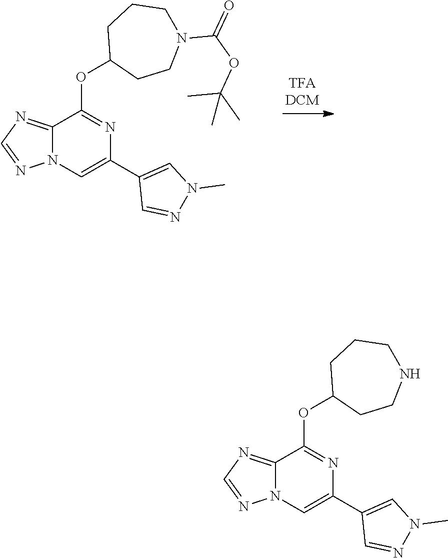 [SMILES: Cn1cc(-c2cn3ncnc3c(OC3CCCN(C(=O)OC(C)(C)C)CC3)n2)cn1 ; Cn1cc(-c2cn3ncnc3c(OC3CCCNCC3)n2)cn1]

A vial containing tert-butyl 4-((6-(1-methyl-1H-pyrazol-4-yl)-[1,2,4]triazolo[1,5-a]pyrazin-8-yl)oxy)azepane-1-carboxylate (708 mg, 1.71 mmol) in anhydrous dichloromethane (1 mL) was cooled in an ice water bath, then trifluoroacetic acid (1 mL, 13.1 mmol) was added carefully dropwise to the cold mixture. Upon complete addition of TFA, the mixture was allowed to warm to 23° C. and monitored with LCMS. After 1 hour, the reaction was carefully concentrated under reduced pressure to afford a light yellow film as 8-(azepan-4-yloxy)-6-(1-methyl-H-pyrazol-4-yl)-[1,2,4]triazolo[1,5-a]pyrazine (301 mg, TFA salt) that was used without further purification. LCMS m/z=314.1 (M+H)⁺.

### Synthesis of 1-(4-((6-(1-methyl-1H-pyrazol-4-yl)-[1,2,4]triazolo[1,5-a]pyrazin-8-yl)oxy)azepan-1-yl)prop-2-en-1-one

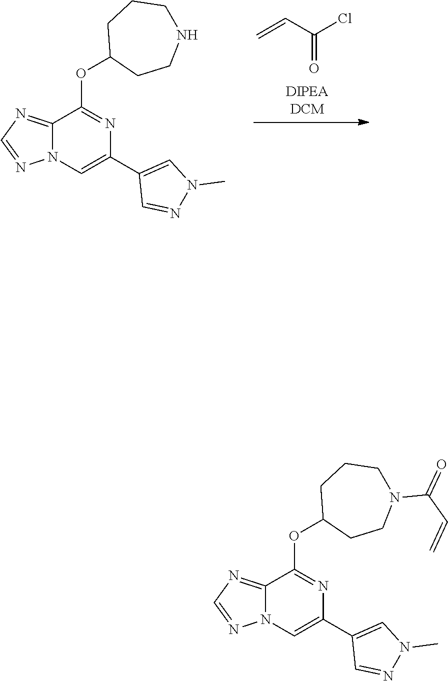

To a vial containing 8-(azepan-4-yloxy)-6-(1-methyl-H-pyrazol-4-yl)-[1,2,4]triazolo[1,5-a]pyrazine (301 mg, 704.5 μmol, TFA salt) in anhydrous dichloromethane (2 mL) was added Hunigs base (0.5 mL, 2.87 mmol) carefully dropwise at −25° C. After 5 minutes, acryloyl chloride (0.1 mL, 1.23 mmol) was added carefully dropwise to the cold homogeneous solution. Upon complete addition of acryloyl chloride, the reaction was allowed to warm to 23° C. and monitored with LCMS and TLC. After 3 minutes, the reaction was carefully quenched with slow addition of saturated aqueous sodium bicarbonate solution. The biphasic mixture was loaded onto a silica gel column and purified with (20-85% [3:1 ethyl acetate: ethanol] in heptanes). The desired fractions were pooled then concentrated under reduced pressure to afford a white solid as 1-(4-((6-(1-methyl-H-pyrazol-4-yl)-[1,2,4]triazolo[1,5-a]pyrazin-8-yl)oxy)azepan-1-yl)prop-2-en-1-one (96 mg, 35% yield). ¹H NMR (500 MHz, DMSO-d₆) d=8.97 (s, 1H), 8.55 (d, J=1.2 Hz, 1H), 8.28-8.21 (m, 1H), 8.04 (d, J=7.3 Hz, 1H), 6.82 (ddd, J=10.4, 12.2, 16.5 Hz, 1H), 6.18 (td, J=2.2, 16.9 Hz, 1H), 5.70 (dd, J=2.4, 10.4 Hz, 1H), 5.53-5.44 (m, 1H), 3.90 (d, J=1.8 Hz, 3H), 3.88-3.61 (m, 3H), 3.59-3.49 (m, 2H), 2.31-2.21 (m, 1H), 2.06-1.97 (m, 3H), 1.83-1.71 (m, 1H). LCMS m/z=368.1 (M+H)⁺.

### Example 58: (R)-1-(6-((6-(1-methyl-1H-pyrazol-4-yl)pyrazolo[1,5-a]pyrazin-4-yl)oxy)-1,4-oxazepan-4-yl)prop-2-en-1-one

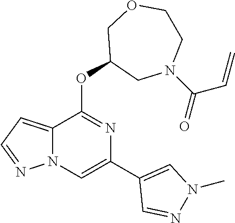 [SMILES: C=CC(=O)N1CCOC[C@H](Oc2nc(-c3cnn(C)c3)cn3nccc23)C1]

### Synthesis of tert-butyl (6R)-6-[6-(1-methylpyrazol-4-yl)pyrazolo[1,5-a]pyrazin-4-yl]oxy-1,4-oxazepane-4-carboxylate

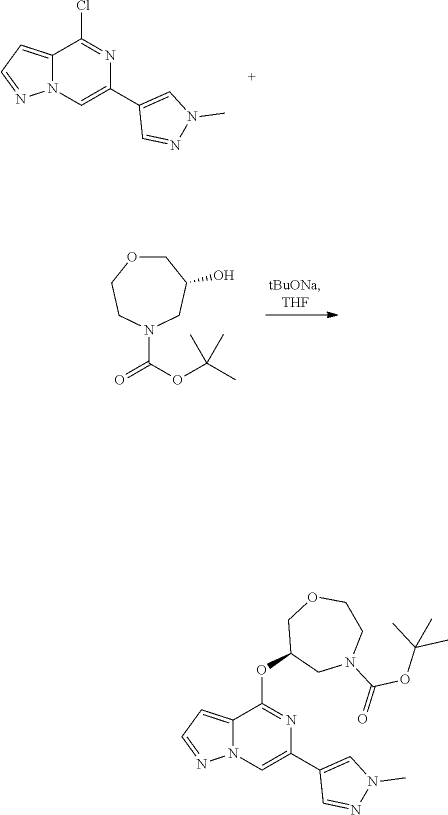 [SMILES: Cn1cc(-c2cn3nccc3c(Cl)n2)cn1 ; CC(C)(C)OC(=O)N1CCOC[C@H](O)C1 ; Cn1cc(-c2cn3nccc3c(O[C@H]3COCCN(C(=O)OC(C)(C)C)C3)n2)cn1]

A flask containing tert-butyl (6R)-6-hydroxy-1,4-oxazepane-4-carboxylate (496 mg, 2.28 mmol) in anhydrous THF (4 mL) was cooled in an ice water bath, then sodium tert-butoxide (308 mg, 3.20 mmol) was added carefully in portions to the cold mixture. After 10 minutes, 4-chloro-6-(1-methylpyrazol-4-yl)pyrazolo[1,5-a]pyrazine (465 mg, 1.99 mmol) was added carefully in portions to the cold heterogeneous mixture. Upon complete addition of 4-chloro-6-(1-methylpyrazol-4-yl)pyrazolo[1,5-a]pyrazine, the mixture was allowed to warm to 23° C. and monitored with LCMS. After 2 hours, the reaction was carefully quenched with slow addition of water then the biphasic mixture was extracted three times with ethyl acetate. The organics were pooled then dried over anhydrous sodium sulfate. After filtration and concentration under reduced pressure, the residue was loaded onto a silica gel column and purified with (30-100% ethyl acetate in heptanes). The desired fractions were pooled then concentrated under reduced pressure to afford a white solid as tert-butyl (6R)-6-[6-(1-methylpyrazol-4-yl)pyrazolo[1,5-a]pyrazin-4-yl]oxy-1,4-oxazepane-4-carboxylate (757.9 mg, 92% yield) that was used without further purification. ¹H NMR (500 MHz, DMSO-d₆) d=8.77 (s, 1H), 8.27-8.19 (m, 1H), 8.08-7.95 (m, 2H), 6.82-6.76 (m, 1H), 5.58 (br d, J=3.7 Hz, 1H), 4.14-3.92 (m, 3H), 3.88 (s, 3H), 3.85-3.80 (m, 1H), 3.79-3.57 (m, 3H), 3.45-3.37 (m, 1H), 1.46-1.04 (m, 9H). LCMS m/z=415.1 (M+H)⁺.

### Synthesis of (6R)-6-[6-(1-methylpyrazol-4-yl)pyrazolo[1,5-a]pyrazin-4-yl]oxy-1,4-oxazepane

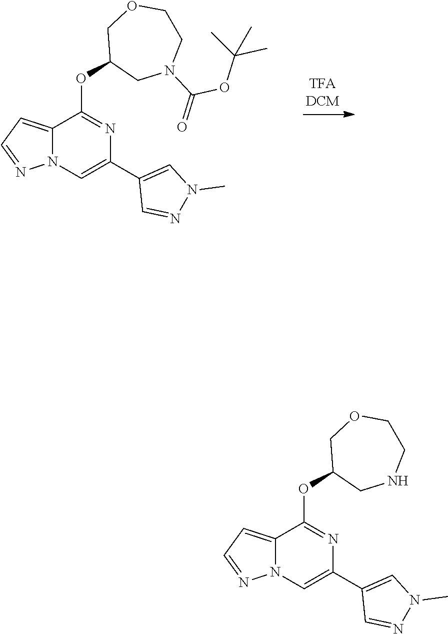 [SMILES: Cn1cc(-c2cn3nccc3c(O[C@H]3COCCN(C(=O)OC(C)(C)C)C3)n2)cn1 ; Cn1cc(-c2cn3nccc3c(O[C@@H]3CNCCOC3)n2)cn1]

A vial containing tert-butyl (6R)-6-[6-(1-methylpyrazol-4-yl)pyrazolo[1,5-a]pyrazin-4-yl]oxy-1,4-oxazepane-4-carboxylate (758 mg, 1.83 mmol) in anhydrous dichloromethane (2 mL) was cooled in an ice water bath, then trifluoroacetic acid (2 mL, 26.1 mmol) was added carefully dropwise to the cold mixture. Upon complete addition of TFA, the mixture was allowed to warm to 23° C. and monitored with LCMS. After 18 hours, the reaction was carefully concentrated under reduced pressure to afford a light yellow film as (6R)-6-[6-(1-methylpyrazol-4-yl)pyrazolo[1,5-a]pyrazin-4-yl]oxy-1,4-oxazepane (783.3 mg, crude, TFA salt) that was used without further purification. LCMS m/z=315.0 (M+H)⁺.

### Synthesis of 1-[(6R)-6-[6-(1-methylpyrazol-4-yl)pyrazolo[1,5-a]pyrazin-4-yl]oxy-1,4-oxazepan-4-yl]prop-2-en-1-one

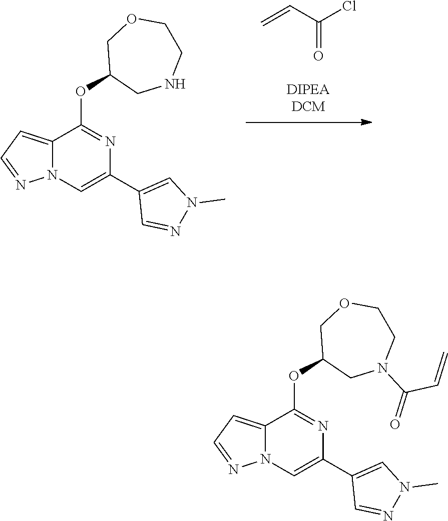

To a vial containing (6R)-6-[6-(1-methylpyrazol-4-yl)pyrazolo[1,5-a]pyrazin-4-yl]oxy-1,4-oxazepane (253 mg, 591 μmol, TFA salt) in anhydrous dichloromethane (2 mL) was added Hunigs base (0.5 mL, 2.87 mmol) carefully dropwise at −25° C. After 5 minutes, acryloyl chloride (0.1 mL, 1.23 mmol) was added carefully dropwise to the cold homogeneous solution. Upon complete addition of acryloyl chloride, the reaction was allowed to warm to 23° C. and monitored with LCMS and TLC. After 3 minutes, the reaction was carefully quenched with slow addition of saturated aqueous sodium bicarbonate solution. The biphasic mixture was loaded onto a silica gel column and purified with (15-100% [3:1 ethyl acetate: ethanol] in heptanes). The desired fractions were pooled then concentrated under reduced pressure to afford a white solid as 1-[(6R)-6-[6-(1-methylpyrazol-4-yl)pyrazolo[1,5-a]pyrazin-4-yl]oxy-1,4-oxazepan-4-yl]prop-2-en-1-one. LCMS: m/z=369.1 (M+H)⁺. ¹H NMR (500 MHz, DMSO-d₆) d=8.80-8.73 (m, 1H), 8.28-8.19 (m, 1H), 8.13-7.99 (m, 2H), 6.92-6.73 (m, 2H), 6.19 (dd, J=2.1, 16.8 Hz, 1H), 5.90-5.59 (m, 3H), 4.56-4.42 (m, 1H), 4.18-4.09 (m, 1H), 4.03-3.92 (m, 2H), 3.90-3.84 (m, 3H), 3.78-3.54 (m, 3H).

### Example 59: (R)-4-((1-acryloylazepan-4-yl)oxy)-6-(1-methyl-1H-pyrazol-4-yl)pyrazolo[1,5-a]pyrazine-3-carbonitrile

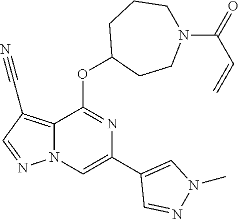 [SMILES: C=CC(=O)N1CCCC(Oc2nc(-c3cnn(C)c3)cn3ncc(C#N)c23)CC1]

### Synthesis of 4-chloro-3-iodo-6-(1-methylpyrazol-4-yl)pyrazolo[1,5-a]pyrazine

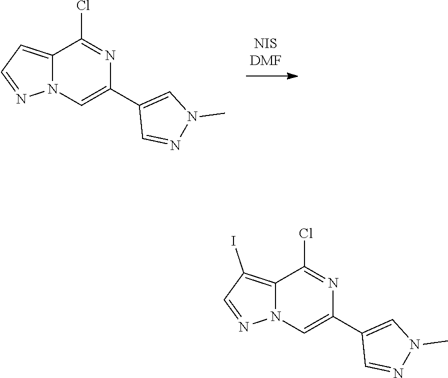 [SMILES: Cn1cc(-c2cn3nccc3c(Cl)n2)cn1 ; Cn1cc(-c2cn3ncc(I)c3c(Cl)n2)cn1]

A vial containing 4-chloro-6-(1-methylpyrazol-4-yl)pyrazolo[1,5-a]pyrazine (477 mg, 2.04 mmol) in anhydrous DMF (5 mL) was cooled in an ice water bath, then N-Iodosuccinimide (1.23 g, 5.48 mmol) was added carefully in portions to the cold mixture. After 15 minutes, the cloudy yellow mixture was heated carefully to 50° C. and monitored with LCMS. After 2 hours, the reaction was cooled to 23° C. and stirred overnight. After 19 hours, the heterogeneous mixture was filtered. The off-white solid was identified as 4-chloro-3-iodo-6-(1-methylpyrazol-4-yl)pyrazolo[1,5-a]pyrazine which was used without purification. ¹H NMR (500 MHz, DMSO-d₆) δ=9.29 (s, 1H), 8.31 (s, 1H), 8.27 (s, 1H), 8.06-8.00 (m, 1H), 3.95-3.84 (m, 3H). LCMS m/z=359.9 (M+H)⁺.

### Synthesis of tert-butyl (4R)-4-[3-iodo-6-(1-methylpyrazol-4-yl)pyrazolo[1,5-a]pyrazin-4-yl]oxyazepane-1-carboxylate

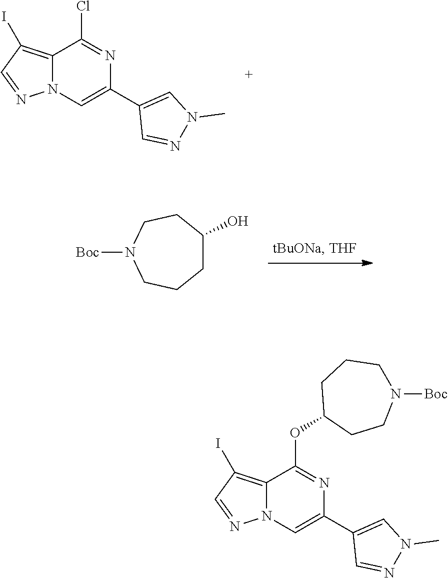 [SMILES: Cn1cc(-c2cn3ncc(I)c3c(Cl)n2)cn1 ; CC(C)(C)OC(=O)N1CCC[C@@H](O)CC1 ; Cn1cc(-c2cn3ncc(I)c3c(O[C@@H]3CCCN(C(=O)OC(C)(C)C)CC3)n2)cn1]

A vial containing tert-butyl (4R)-4-hydroxyazepane-1-carboxylate (113 mg, 525 μmol) in anhydrous THF (2 mL) was cooled in an ice water bath, then sodium tert-butoxide (79.5 mg, 827 mol) was added carefully in portions to the cold mixture. After 10 minutes, 4-chloro-3-iodo-6-(1-methylpyrazol-4-yl)pyrazolo[1,5-a]pyrazine (181 mg, 504 μmol) was added carefully in portions to the cold heterogeneous mixture. Upon complete addition of 4-chloro-3-iodo-6-(1-methylpyrazol-4-yl)pyrazolo[1,5-a]pyrazine, the mixture was allowed to warm to 23° C. and monitored with LCMS. After 18 hours, the heterogeneous reaction was carefully concentrated under reduced pressure. The residue was diluted with ethyl acetate then washed with saturated aqueous sodium chloride solution. The organic layer was dried over anhydrous sodium sulfate. After filtration and concentration under reduced pressure, the residue was loaded onto a silica gel column and purified with (20-65% ethyl acetate in heptanes). The desired fractions were pooled then concentrated under reduced pressure to afford a colorless sticky film as tert-butyl (4R)-4-[3-iodo-6-(1-methylpyrazol-4-yl)pyrazolo[1,5-a]pyrazin-4-yl]oxyazepane-1-carboxylate (99.3 mg, 36% yield) that was used without further purification. ¹H NMR (400 MHz, DMSO-d₆) δ=8.78 (s, 1H), 8.19 (d, J=6.0 Hz, 1H), 8.09 (s, 1H), 7.99 (d, J=2.5 Hz, 1H), 5.59 (br s, 1H), 3.88 (s, 3H), 3.72-3.38 (m, 4H), 2.09-1.97 (m, 3H), 1.83-1.71 (m, 2H), 1.55-1.46 (m, 1H), 1.42 (d, J=5.0 Hz, 9H). LCMS m/z=539.0 (M+H)⁺.

### Synthesis of tert-butyl (4R)-4-[3-cyano-6-(1-methylpyrazol-4-yl)pyrazolo[1,5-a]pyrazin-4-yl]oxyazepane-1-carboxylate

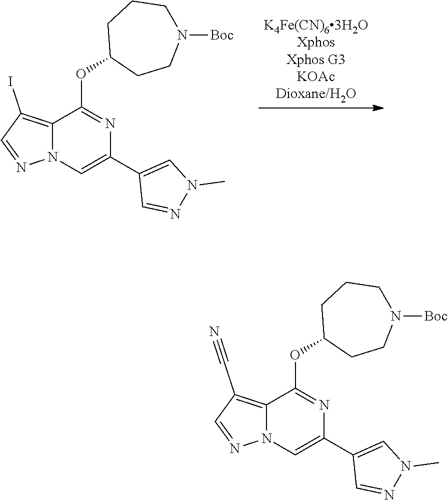 [SMILES: Cn1cc(-c2cn3ncc(I)c3c(O[C@@H]3CCCN(C(=O)OC(C)(C)C)CC3)n2)cn1 ; Cn1cc(-c2cn3ncc(C#N)c3c(O[C@@H]3CCCN(C(=O)OC(C)(C)C)CC3)n2)cn1]

A vial containing tert-butyl (4R)-4-[3-iodo-6-(1-methylpyrazol-4-yl)pyrazolo[1,5-a]pyrazin-4-yl]oxyazepane-1-carboxylate (106 mg, 196 μmol), potassium hexacyanoferrate (II) trihydrate (44 mg, 104 mol), dicyclohexyl-[2-(2,4,6-triisopropylphenyl)phenyl]phosphane, (Xphos) (10 mg, 21 μmol), [2-(2-aminophenyl)phenyl]-methylsulfonyloxy-palladium; dicyclohexyl-[2-(2,4,6-triisopropylphenyl)phenyl]phosphane (Xphos G3) (17 mg, 20 μmol), and potassium acetate (40 mg, 408 mol) in dioxane (1 mL) and water (1 mL) was degassed and backfilled with nitrogen. (Repeated evacuation and nitrogen backfill three times.) The heterogeneous white reaction mixture was carefully heated to 90° C. and monitored with LCMS. After 18 hours, the heterogeneous reaction was cooled to room temperature then carefully partitioned between water and ethyl acetate. The aqueous layer was extracted two additional times with ethyl acetate. The organic extractions were pooled then washed once saturated aqueous sodium chloride solution, then the organic layer was dried over anhydrous sodium sulfate. After filtration and concentration under reduced pressure, the residue was loaded onto a silica gel column and purified with (25-80% ethyl acetate in heptanes). The desired fractions were pooled then concentrated under reduced pressure to afford a colorless sticky film as tert-butyl (4R)-4-[3-cyano-6-(1-methylpyrazol-4-yl)pyrazolo[1,5-a]pyrazin-4-yl]oxyazepane-1-carboxylate (10.2 mg, 12% yield) that was used without further purification. LCMS m/z=460.1 (M+Na)⁺.

### Synthesis of 4-[(4R)-azepan-4-yl]oxy-6-(1-methylpyrazol-4-yl)pyrazolo[1,5-a]pyrazine-3-carbonitrile

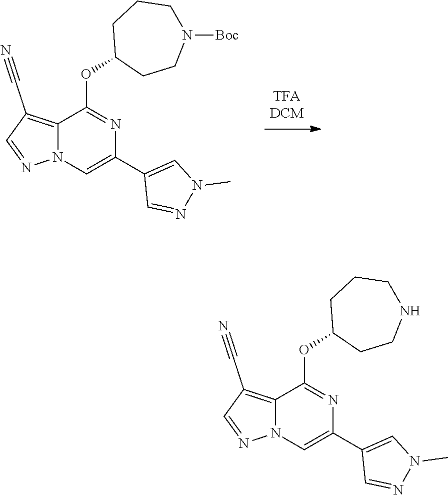 [SMILES: Cn1cc(-c2cn3ncc(C#N)c3c(O[C@@H]3CCCN(C(=O)OC(C)(C)C)CC3)n2)cn1 ; Cn1cc(-c2cn3ncc(C#N)c3c(O[C@@H]3CCCNCC3)n2)cn1]

A vial containing tert-butyl (4R)-4-[3-cyano-6-(1-methylpyrazol-4-yl)pyrazolo[1,5-a]pyrazin-4-yl]oxyazepane-1-carboxylate (10 mg, 23 μmol) in anhydrous dichloromethane (0.5 mL) was cooled in an ice water bath, then TFA (0.05 mL, 653 μmol) was added carefully dropwise to the cold mixture. Upon complete addition of TFA, the mixture was allowed to warm to 23° C. and monitored with LCMS. After 15 minutes, the reaction was carefully concentrated under reduced pressure to afford a light yellow film as 4-[(4R)-azepan-4-yl]oxy-6-(1-methylpyrazol-4-yl)pyrazolo[1,5-a]pyrazine-3-carbonitrile (11 mg, trifluoroacetic acid salt) that was used without purification. LCMS m/z=338.1 (M+H)⁺.

### Synthesis of 6-(1-methylpyrazol-4-yl)-4-[(4R)-1-prop-2-enoylazepan-4-yl]oxy-pyrazolo[1,5-a]pyrazine-3-carbonitrile

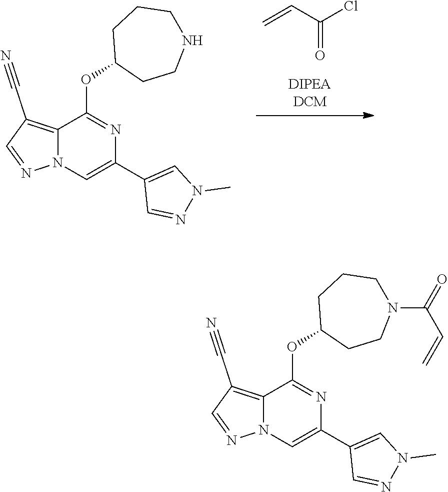

To a vial containing 4-[(4R)-azepan-4-yl]oxy-6-(1-methylpyrazol-4-yl)pyrazolo[1,5-a]pyrazine-3-carbonitrile (11 mg, 24.4 μmol, trifluoroacetic acid salt) in anhydrous THF (0.5 mL) was added Hunigs base (0.1 mL 574 μmol) carefully dropwise at −25° C. After 5 minutes, acryloyl chloride (0.01 mL, 123 μmol) was added carefully dropwise to the cold homogeneous solution. Upon complete addition of acryloyl chloride, the reaction was allowed to warm to 23° C. and monitored with LCMS and TLC. After 3 minutes, the reaction was carefully quenched with slow addition of saturated aqueous sodium bicarbonate solution. The biphasic mixture was loaded onto a silica gel column and purified with (25-85% [3:1 ethyl acetate: ethanol] in heptane.) The desired fractions were pooled then concentrated under reduced pressure to afford a colorless film as 6-(1-methylpyrazol-4-yl)-4-[(4R)-1-prop-2-enoylazepan-4-yl]oxy-pyrazolo[1,5-a]pyrazine-3-carbonitrile. ¹H NMR (400 MHz, DICHLOROMETHANE-d₂) δ=8.30-8.16 (m, 2H), 7.96-7.89 (m, 1H), 7.88-7.87 (m, 1H), 6.75-6.61 (m, 1H), 6.33-6.25 (m, 1H), 5.71-5.64 (m, 2H), 4.16-3.95 (m, 4H), 3.86-3.54 (m, 3H), 3.49-3.34 (m, 1H), 2.35-2.13 (m, 4H), 1.96 (br d, J=11.5 Hz, 1H). LCMS m/z=392.1 (M+H)⁺.

### Example 60: N-methyl-N-((trans)-3-((3-methyl-6-(1-methyl-1H-pyrazol-4-yl)pyrazolo[1,5-a]pyrazin-4-yl)oxy)cyclobutyl)acrylamide

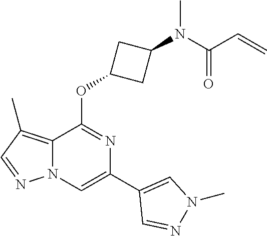 [SMILES: C=CC(=O)N(C)[C@H]1C[C@H](Oc2nc(-c3cnn(C)c3)cn3ncc(C)c23)C1]

### Synthesis of tert-butyl ((trans)-3-((3-iodo-6-(1-methyl-1H-pyrazol-4-yl)pyrazolo[1,5-a]pyrazin-4-yl)oxy)cyclobutyl)(methyl)carbamate

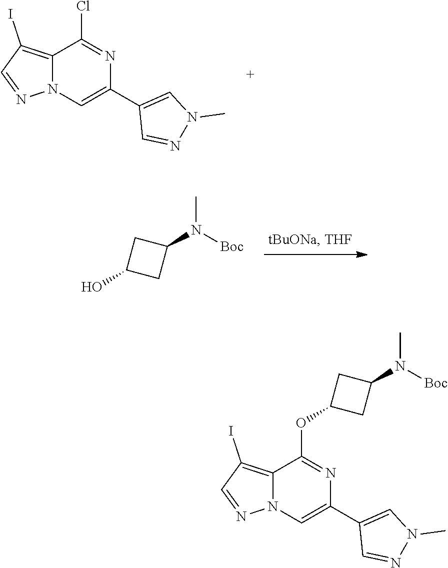 [SMILES: Cn1cc(-c2cn3ncc(I)c3c(Cl)n2)cn1 ; CN(C(=O)OC(C)(C)C)[C@H]1C[C@H](O)C1 ; Cn1cc(-c2cn3ncc(I)c3c(O[C@H]3C[C@H](N(C)C(=O)OC(C)(C)C)C3)n2)cn1]

A vial containing racemic trans tert-butyl N-(3-hydroxycyclobutyl)-N-methyl-carbamate (406 mg, 2.0 mmol) in anhydrous THF (6 mL) was cooled in an ice water bath, then sodium tert-butoxide (333 mg, 3.47 mmol) was added carefully in portions to the cold mixture. After 10 minutes, 4-chloro-3-iodo-6-(1-methylpyrazol-4-yl)pyrazolo[1,5-a]pyrazine (593 mg, 1.65 mmol) was added carefully to the cold mixture. Upon complete addition of 4-chloro-3-iodo-6-(1-methylpyrazol-4-yl)pyrazolo[1,5-a]pyrazine, the mixture was allowed to warm to 23° C. After 1 hour, the reaction was carefully quenched with slow addition of saturated aqueous sodium bicarbonate solution, then the biphasic mixture was extracted three times with ethyl acetate. The organics were pooled then dried over anhydrous sodium sulfate. After filtration and concentration under reduced pressure, the residue was loaded onto a silica gel column and purified with (15-70% ethyl acetate in heptanes). The desired fractions were pooled then concentrated under reduced pressure to afford a white solid as tert-butyl ((trans)-3-((3-iodo-6-(1-methyl-1H-pyrazol-4-yl)pyrazolo[1,5-a]pyrazin-4-yl)oxy)cyclobutyl)(methyl)carbamate (698.5 mg, crude) that was used without further purification. ¹H NMR (500 MHz, DMSO-d₆) δ=8.79 (s, 1H), 8.16 (s, 1H), 8.11 (s, 1H), 7.98 (s, 1H), 5.45 (br t, J=6.7 Hz, 1H), 4.89 (br s, 1H), 3.88 (s, 3H), 2.89-2.84 (m, 3H), 2.79-2.72 (m, 2H), 2.48-2.42 (m, 2H), 1.41 (s, 9H). LCMS: m/z=525.0 (M+H)⁺.

### Synthesis of tert-butyl methyl((trans)-3-((3-methyl-6-(1-methyl-1H-pyrazol-4-yl)pyrazolo[1,5-a]pyrazin-4-yl)oxy)cyclobutyl)carbamate

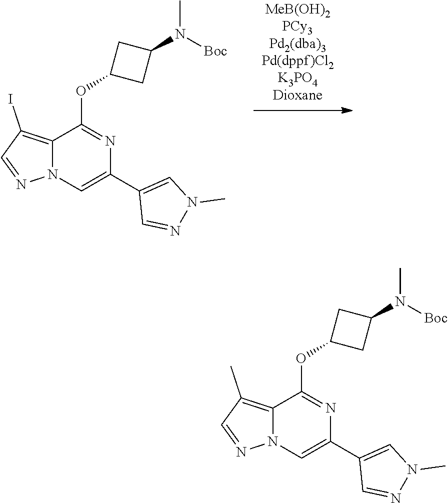 [SMILES: Cn1cc(-c2cn3ncc(I)c3c(O[C@H]3C[C@H](N(C)C(=O)OC(C)(C)C)C3)n2)cn1 ; Cc1cnn2cc(-c3cnn(C)c3)nc(O[C@H]3C[C@H](N(C)C(=O)OC(C)(C)C)C3)c12]

A vial containing tert-butyl ((trans)-3-((3-iodo-6-(1-methyl-1H-pyrazol-4-yl)pyrazolo[1,5-a]pyrazin-4-yl)oxy)cyclobutyl)(methyl)carbamate (204 mg, 389 μmol), methylboronic acid (81 mg, 1.35 mmol), tricyclohexylphosphane (29 mg, 105 μmol), Pd₂(dba)₃ (36 mg, 39 μmol), Pd(dppf)Cl₂·CH₂Cl₂ (68 mg, 84 μmol) and potassium phosphate tribasic (1.0 M solution, 1.2 mL) in dioxane (4 mL) was degassed and backfilled with nitrogen. Repeated evacuation and nitrogen backfill were executed three times. The heterogeneous reaction mixture was carefully heated to 90° C. After 18 hours, the heterogeneous reaction was cooled to room temperature then carefully partitioned between water and ethyl acetate. The aqueous layer was extracted two additional times with ethyl acetate. The organic extractions were pooled then washed once saturated aqueous sodium chloride solution, then the organic layer was dried over anhydrous sodium sulfate. After filtration and concentration under reduced pressure, the residue was loaded onto a silica gel column and purified with (30-90% ethyl acetate in heptanes). The desired fractions were pooled then concentrated under reduced pressure to afford a dark yellow film as tert-butyl methyl((trans)-3-((3-methyl-6-(1-methyl-1H-pyrazol-4-yl)pyrazolo[1,5-a]pyrazin-4-yl)oxy)cyclobutyl)carbamate (125.7 mg, 78% yield) that was used without further purification. LCMS m/z=413.2 (M+H)⁺.

### Synthesis of (trans)-N-methyl-3-((3-methyl-6-(1-methyl-1H-pyrazol-4-yl)pyrazolo[1,5-a]pyrazin-4-yl)oxy)cyclobutan-1-amine

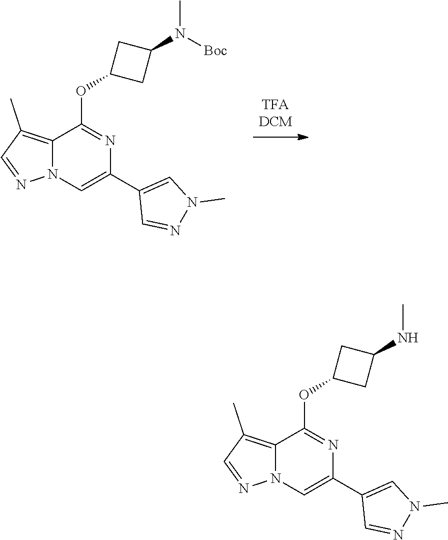 [SMILES: Cc1cnn2cc(-c3cnn(C)c3)nc(O[C@H]3C[C@H](N(C)C(=O)OC(C)(C)C)C3)c12 ; CN[C@H]1C[C@H](Oc2nc(-c3cnn(C)c3)cn3ncc(C)c23)C1]

A vial containing tert-butyl methyl((trans)-3-((3-methyl-6-(1-methyl-1H-pyrazol-4-yl)pyrazolo[1,5-a]pyrazin-4-yl)oxy)cyclobutyl)carbamate (125.7 mg, 305 μmol) in anhydrous dichloromethane (2 mL) was cooled in an ice water bath, then trifluoroacetic acid (0.23 mL, 3 mmol) was added carefully dropwise to the cold mixture. Upon complete addition of TFA, the mixture was allowed to warm to 23° C. After 1.5 hours, the reaction was carefully concentrated under reduced pressure to afford a light yellow film as (trans)-N-methyl-3-((3-methyl-6-(1-methyl-1H-pyrazol-4-yl)pyrazolo[1,5-a]pyrazin-4-yl)oxy)cyclobutan-1-amine trifluoroacetate (133.9 mg, crude) that was used without purification. LCMS m/z=313.1 (M+H)⁺.

### Synthesis of N-methyl-N-((trans)-3-((3-methyl-6-(1-methyl-1H-pyrazol-4-yl)pyrazolo[1,5-a]pyrazin-4-yl)oxy)cyclobutyl)acrylamide

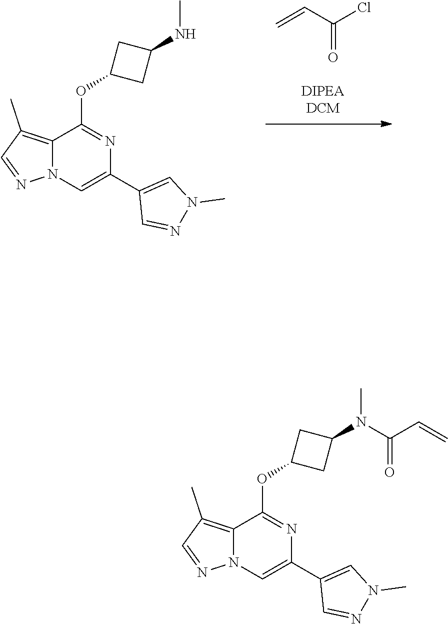

To a vial containing (trans)-N-methyl-3-((3-methyl-6-(1-methyl-1H-pyrazol-4-yl)pyrazolo[1,5-a]pyrazin-4-yl)oxy)cyclobutan-1-amine trifluoroacetate (133.9 mg, 314 μmol) in anhydrous THF (3 mL) was added Hunigs base (1 mL, 5.74 mmol) carefully dropwise at −25° C. After 5 minutes, acryloyl chloride (0.05 mL, 615 μmol) was added carefully dropwise to the cold homogeneous solution. Upon complete addition of acryloyl chloride, the reaction was allowed to warm to 23° C. and monitored with LCMS and TLC. After 3 minutes, the reaction was carefully quenched with slow addition of saturated aqueous sodium bicarbonate solution. The mixture was stirred at 23° C. for 1 hour, then the biphasic mixture was extracted three times with ethyl acetate. The organics were pooled and washed once with saturated aqueous sodium bicarbonate solution. The organic layer was separated then dried over anhydrous sodium sulfate. After filtration and concentration under reduced pressure, the residue was loaded onto a silica gel column and purified with (15-65% [3:1 ethyl acetate: ethanol] in heptanes). The desired fractions were pooled then concentrated under reduced pressure to afford a white foam that was diluted with DMSO then filtered. The homogeneous mixture was purified with reverse-phase mass directed HPLC purification. (Liquid chromatography was performed using a Waters XSelect CSH C18, 5 m, 19 mm×100 mm column with mobile phase H₂O (A) and MeCN (B) and a gradient of 5-60% B (0.2% NH₄OH final v/v % modifier) with flow rate at 30 mL/min.) Fractions containing desired product were pooled then concentrated to afford a colorless film as N-methyl-N-((trans)-3-((3-methyl-6-(1-methyl-1H-pyrazol-4-yl)pyrazolo[1,5-a]pyrazin-4-yl)oxy)cyclobutyl)-acrylamide. ¹H NMR (500 MHz, DMSO-d₆) δ=8.62 (s, 1H), 8.14 (s, 1H), 7.97 (s, 1H), 7.84 (s, 1H), 6.76 (br dd, J=11.0, 16.5 Hz, 1H), 6.09 (br s, 1H), 5.68 (br s, 1H), 5.49 (br s, 1H), 5.35-4.90 (m, 1H), 3.88 (s, 3H), 3.15-2.77 (m, 5H), 2.67-2.52 (m, 2H), 2.47 (s, 3H). LCMS m/z=367.2 (M+H)⁺.

### Example 61: 1-[(4R)-4-[3-fluoro-6-(1-methylpyrazol-4-yl)pyrazolo[1,5-a]pyrazin-4-yl]oxyazepan-1-yl]prop-2-en-1-one

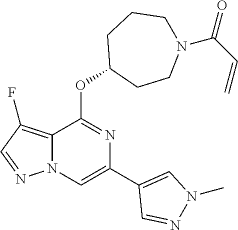 [SMILES: C=CC(=O)N1CCC[C@@H](Oc2nc(-c3cnn(C)c3)cn3ncc(F)c23)CC1]

### Synthesis of tert-butyl (4R)-4-[3-fluoro-6-(1-methylpyrazol-4-yl)pyrazolo[1,5-a]pyrazin-4-yl]oxyazepane-1-carboxylate

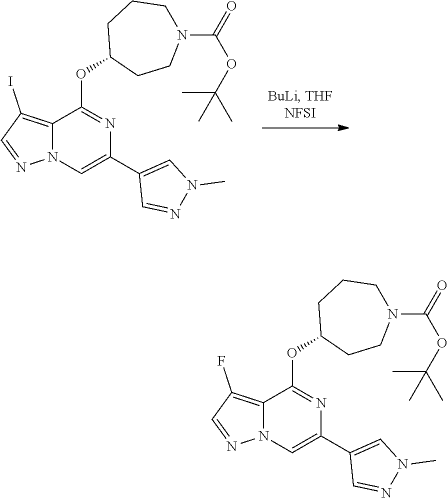 [SMILES: Cn1cc(-c2cn3ncc(I)c3c(O[C@@H]3CCCN(C(=O)OC(C)(C)C)CC3)n2)cn1 ; Cn1cc(-c2cn3ncc(F)c3c(O[C@@H]3CCCN(C(=O)OC(C)(C)C)CC3)n2)cn1]

A solution of tert-butyl (4R)-4-[3-iodo-6-(1-methylpyrazol-4-yl)pyrazolo[1,5-a]pyrazin-4-yl]oxyazepane-1-carboxylate (224 mg, 416 μmol) in THF (3 mL) was cooled to −78° C., and butyllithium (2.5 M, 200 μL) was added and stirred for 20 min. Then N-fluorobenzenesulfonimide (157 mg, 499 μmol) in THF was added and the reaction mixture was stirred for 1 h. Aqueous NH₄Cl was added to quench the reaction. The reaction was diluted with EtOAc, the layers were separated, and the aqueous layer extracted with EtOAc. The combined organic layers were dried over Na₂SO₄ and the concentrated residue was purified (SiO₂, 0-70% EtOAc/DCM) to provide tert-butyl (4R)-4-[3-fluoro-6-(1-methylpyrazol-4-yl)pyrazolo[1,5-a]pyrazin-4-yl]oxyazepane-1-carboxylate (25.3 mg, 14% yield) as an amorphous solid. LCMS: Rt=0.93 min, m/z 431.2. ¹H NMR (400 MHz, CHLOROFORM-d) δ 7.99 (d, J=1.51 Hz, 1H), 7.83 (s, 1H), 7.74-7.83 (m, 1H), 7.70 (d, J=3.76 Hz, 1H), 5.52-5.61 (m, 1H), 3.97 (s, 3H), 3.51-3.82 (m, 2H), 3.25-3.48 (m, 2H), 2.07-2.17 (m, 3H), 1.98 (br d, J=12.05 Hz, 2H), 1.78 (br d, J=5.02 Hz, 1H), 1.49 (s, 9H). ¹⁹F NMR (376 MHz, CHLOROFORM-d) S-174.27 (s, 1F).

### Synthesis of 4-[(4R)-azepan-4-yl]oxy-3-fluoro-6-(1-methylpyrazol-4-yl)pyrazolo[1,5-a]pyrazine

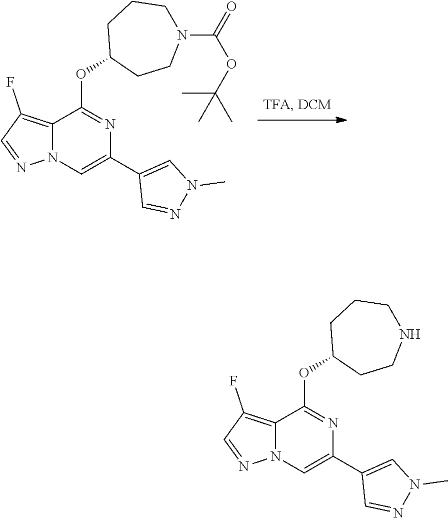 [SMILES: Cn1cc(-c2cn3ncc(F)c3c(O[C@@H]3CCCN(C(=O)OC(C)(C)C)CC3)n2)cn1 ; Cn1cc(-c2cn3ncc(F)c3c(O[C@@H]3CCCNCC3)n2)cn1]

To a solution of tert-butyl (4R)-4-[3-fluoro-6-(1-methylpyrazol-4-yl)pyrazolo[1,5-a]pyrazin-4-yl]oxyazepane-1-carboxylate (25 mg, 59 μmol) in DCM (1 mL) was added TFA (1.49 g, 13.1 mmol, 1 mL) and stirred at RT for 1 h. After concentration, the crude residue 4-[(4R)-azepan-4-yl]oxy-3-fluoro-6-(1-methylpyrazol-4-yl)pyrazolo[1,5-a]pyrazine was used as is for the next step. LCMS: Rt=0.63 min, m/z 183.2.

### Synthesis of 1-[(4R)-4-[3-fluoro-6-(1-methylpyrazol-4-yl)pyrazolo[1,5-a]pyrazin-4-yl]oxyazepan-1-yl]prop-2-en-1-one

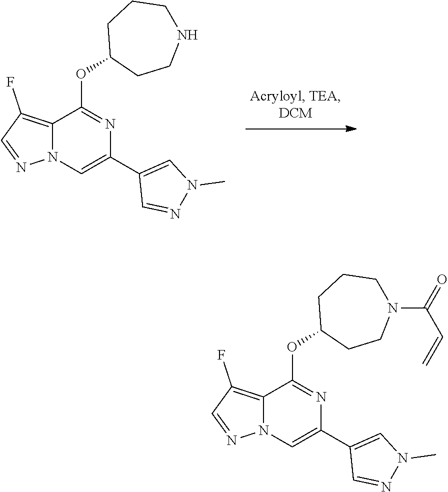 [SMILES: Cn1cc(-c2cn3ncc(F)c3c(O[C@@H]3CCCNCC3)n2)cn1 ; C=CC(=O)N1CCC[C@@H](Oc2nc(-c3cnn(C)c3)cn3ncc(F)c23)CC1]

To a solution of 4-[(4R)-azepan-4-yl]oxy-3-fluoro-6-(1-methylpyrazol-4-yl)pyrazolo[1,5-a]pyrazine (48 mg, 146 μmol) in DCM (4 mL) was added TEA (30 mg, 292 μmol, 41 μL) and the reaction was stirred for 5 min. After cooling to 0° C., acryloyl chloride (16 mg, 175 μmol, 14 μL) was added and stirred for 3 min. The reaction was quenched with saturated aqueous NaHCO₃ and extracted with DCM. The organic layer was dried over Na₂SO₄ and the concentrated residue was chromatographed on silica gel (EtOAc/MeOH 0-30%) to give 1-[(4R)-4-[3-fluoro-6-(1-methylpyrazol-4-yl)pyrazolo[1,5-a]pyrazin-4-yl]oxyazepan-1-yl]prop-2-en-1-one (12.6 mg, 21% yield, 95% purity). LCMS: Rt=0.70 min, m/z 385.0. ¹H NMR (400 MHz, CHLOROFORM-d) δ 8.01 (d, J=1.51 Hz, 1H), 7.85 (br s, 1H), 7.76 (s, 1H), 7.71 (d, J=3.76 Hz, 1H), 6.58-6.68 (m, 1H), 6.35-6.44 (m, 1H), 5.73 (br d, J=10.54 Hz, 1H), 5.62 (br s, 1H), 3.99 (br d, J=2.51 Hz, 3H), 3.79 (br dd, J=6.78, 13.55 Hz, 1H), 3.62-3.73 (m, 1H), 3.52-3.61 (m, 1H), 3.41-3.51 (m, 1H), 2.16-2.29 (m, 4H), 1.74-2.00 (m, 2H). ¹⁹F NMR (376 MHz, CHLOROFORM-d) δ-174.12 (br d, J=58.58 Hz, 1F).

### Example 62: 1-[(4R)-4-[6-(1-methylpyrazol-4-yl)-3-(trifluoromethyl)pyrazolo[1,5-a]pyrazin-4-yl]oxyazepan-1-yl]prop-2-en-1-one

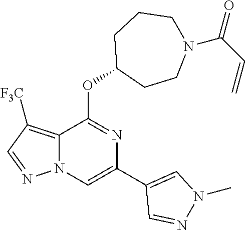 [SMILES: C=CC(=O)N1CCC[C@@H](Oc2nc(-c3cnn(C)c3)cn3ncc(C(F)(F)F)c23)CC1]

### Synthesis of tert-butyl (4R)-4-[6-(1-methylpyrazol-4-yl)-3-(trifluoromethyl)pyrazolo[1,5-a]pyrazin-4-yl]oxyazepane-1-carboxylate

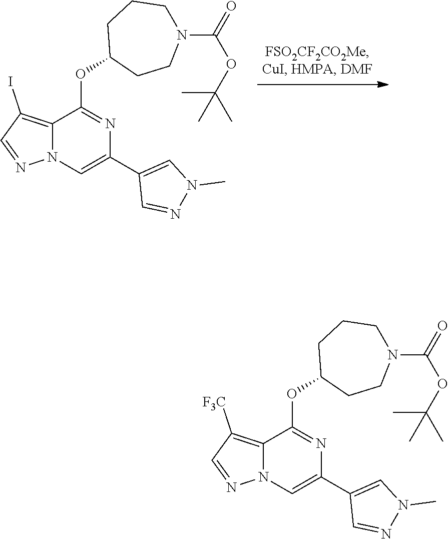 [SMILES: Cn1cc(-c2cn3ncc(I)c3c(O[C@@H]3CCCN(C(=O)OC(C)(C)C)CC3)n2)cn1 ; Cn1cc(-c2cn3ncc(C(F)(F)F)c3c(O[C@@H]3CCCN(C(=O)OC(C)(C)C)CC3)n2)cn1]

To a solution of tert-butyl (4R)-4-[3-iodo-6-(1-methylpyrazol-4-yl)pyrazolo[1,5-a]pyrazin-4-yl]oxyazepane-1-carboxylate (125 mg, 232 μmol) in DMF (1 mL) and hexamethylphosphoramide (42 mg, 232 μmol, 40 μL) was added methyl fluorosulphonyldifluoroacetate (223 mg, 1.16 mmol, 148 L) and copper(I) iodide (66 mg, 348 μmol) and degassed. The mixture was heated at 80° C. overnight. The cooled mixture was diluted with EtOAc, washed with aqueous NH₄Cl and filtrated through Celite and dried over Na₂SO₄. The concentrated residue was chromatographed on silica gel (heptanes/EtOAc 0-70%) to give tert-butyl (4R)-4-[6-(1-methylpyrazol-4-yl)-3-(trifluoromethyl)pyrazolo[1,5-a]pyrazin-4-yl]oxyazepane-1-carboxylate (18 mg, 15% yield, 95% purity). LCMS: Rt=1.03 min, m/z 481.2. 381.2.

### Synthesis of 4-[(4R)-azepan-4-yl]oxy-6-(1-methylpyrazol-4-yl)-3-(trifluoromethyl)pyrazolo[1,5-a]pyrazine

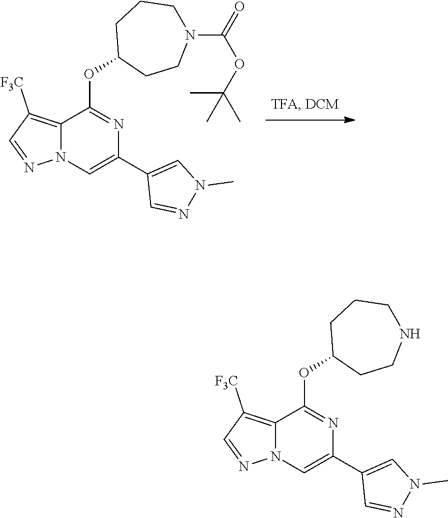 [SMILES: Cn1cc(-c2cn3ncc(C(F)(F)F)c3c(O[C@@H]3CCCN(C(=O)OC(C)(C)C)CC3)n2)cn1 ; Cn1cc(-c2cn3ncc(C(F)(F)F)c3c(O[C@@H]3CCCNCC3)n2)cn1]

To a solution of tert-butyl (4R)-4-[6-(1-methylpyrazol-4-yl)-3-(trifluoromethyl)pyrazolo[1,5-a]pyrazin-4-yl]oxyazepane-1-carboxylate (18 mg, 37 μmol) in DCM (1 mL) was added TFA (1.49 g, 13.1 mmol, 1 mL) and the reaction mixture was stirred at RT for 1 h. The crude was concentrated to give 4-[(4R)-azepan-4-yl]oxy-6-(1-methylpyrazol-4-yl)-3-(trifluoromethyl)pyrazolo[1,5-a]pyrazine (19.0 mg, crude, trifluoroacetic acid) as a residue and used as is for the next step. LCMS: Rt=0.64 min, m/z 381.2.

### Synthesis of 1-[(4R)-4-[6-(1-methylpyrazol-4-yl)-3-(trifluoromethyl)pyrazolo[1,5-a]pyrazin-4-yl]oxyazepan-1-yl]prop-2-en-1-one

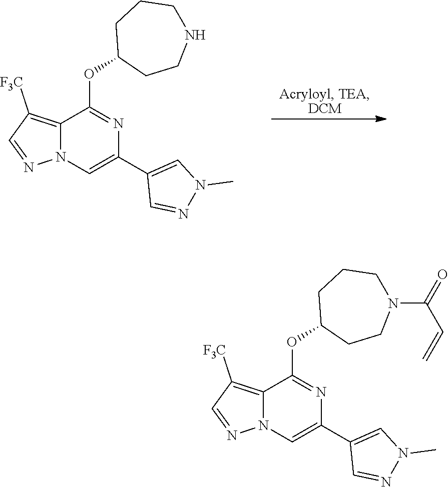 [SMILES: Cn1cc(-c2cn3ncc(C(F)(F)F)c3c(O[C@@H]3CCCNCC3)n2)cn1 ; "Acryloyl, TEA,DCM" ; C=CC(=O)N1CCC[C@@H](Oc2nc(-c3cnn(C)c3)cn3ncc(C(F)(F)F)c23)CC1]

To a solution of 4-[(4R)-azepan-4-yl]oxy-6-(1-methylpyrazol-4-yl)-3-(trifluoromethyl)-pyrazolo[1,5-a]pyrazine (19 mg, 38 μmol, trifluoroacetic acid) in DCM (2 mL) was added TEA (7.8 mg, 77 μmol, 11 μL) and the reaction mixture was stirred for 5 min. After cooling to 0° C., acryloyl chloride (4.2 mg, 46 μmol, 3.8 μL) was added and the reaction mixture was stirred for 3 min. The reaction was quenched with saturated aqueous NaHCO₃ and extracted with DCM. The organic layer was dried over Na₂SO₄ and the concentrated residue was chromatographed on silica gel (EtOAc/MeOH 0-30%) to give 1-[(4R)-4-[6-(1-methylpyrazol-4-yl)-3-(trifluoromethyl)pyrazolo[1,5-a]pyrazin-4-yl]oxyazepan-1-yl]prop-2-en-1-one (9 mg, 51% yield, 95% purity). LCMS: Rt=0.80 min, m/z 457.1 [M+Na]+. ¹H NMR (400 MHz, CHLOROFORM-d) δ 8.24 (s, 1H), 8.11 (s, 1H), 7.88 (s, 1H), 7.78-7.87 (m, 1H), 6.58-6.70 (m, 1H), 6.39 (br t, J=15.18 Hz, 1H), 5.73 (br d, J=10.54 Hz, 1H), 5.67 (br s, 1H), 4.11-4.22 (m, 1H), 4.00 (s, 3H), 3.73-3.83 (m, 1H), 3.52-3.70 (m, 1H), 3.37 (br dd, J=5.52, 14.31 Hz, 1H), 2.18-2.38 (m, 4H), 1.86-1.95 (m, 2H). ¹⁹F NMR (376 MHz, CHLOROFORM-d) d −54.80 (d, J=5.45 Hz, 3F).

### Example 63: N-((cis)-3-((3-fluoro-6-(1-methyl-1H-pyrazol-4-yl)pyrazolo[1,5-a]pyrazin-4-yl)oxy)cyclobutyl)-N-methylacrylamide

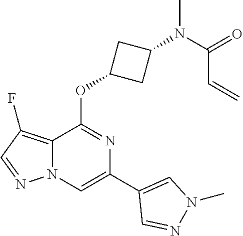 [SMILES: C=CC(=O)N(C)[C@H]1C[C@@H](Oc2nc(-c3cnn(C)c3)cn3ncc(F)c23)C1]

### Synthesis of tert-butyl ((cis)-3-((3-iodo-6-(1-methyl-1H-pyrazol-4-yl)pyrazolo[1,5-a]pyrazin-4-yl)oxy)cyclobutyl)(methyl)carbamate

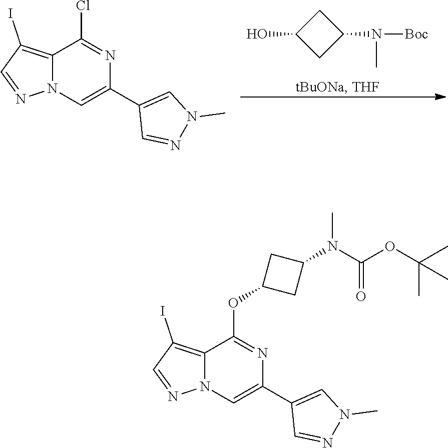

A vial containing tert-butyl ((cis)-3-hydroxycyclobutyl)(methyl)carbamate (175 mg, 868 mol) in anhydrous THF (10 mL) was cooled in an ice water bath, then sodium tert-butoxide (132 mg, 1.37 mmol) was added carefully in portions to the cold mixture. After 10 minutes, 4-chloro-3-iodo-6-(1-methylpyrazol-4-yl)pyrazolo[1,5-a]pyrazine (300 mg, 834 μmol) was added carefully in portions to the cold heterogeneous mixture. Upon complete addition of 4-chloro-3-iodo-6-(1-methylpyrazol-4-yl)pyrazolo[1,5-a]pyrazine, the mixture was allowed to warm to 23° C. and monitored with LCMS. After 18 hours, the residue was diluted with ethyl acetate, and filtrated through Celite. The concentrated residue was loaded onto a silica gel column and purified with (20-65% ethyl acetate in heptanes) to give tert-butyl ((cis)-3-((3-iodo-6-(1-methyl-1H-pyrazol-4-yl)pyrazolo[1,5-a]pyrazin-4-yl)oxy)cyclobutyl)(methyl)carbamate (252 mg, 58% yield). LCMS: Rt=1.00 min, m/z 525.2.

### Synthesis of tert-butyl ((cis)-3-((3-fluoro-6-(1-methyl-1H-pyrazol-4-yl)pyrazolo[1,5-a]pyrazin-4-yl)oxy)cyclobutyl)(methyl)carbamate

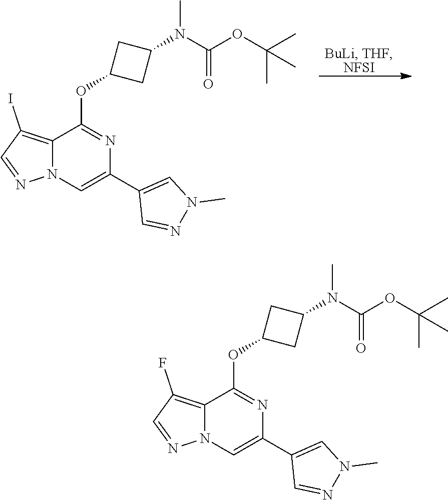

A solution of tert-butyl ((cis)-3-((3-iodo-6-(1-methyl-1H-pyrazol-4-yl)pyrazolo[1,5-a]pyrazin-4-yl)oxy)cyclobutyl)(methyl)carbamate (292 mg, 557 μmol) in THF (3 mL) was cooled to −78° C., and butyllithium (2.5 M, 267.30 μL) was added and the reaction mixture was stirred for 20 min. Then N-fluorobenzenesulfonimide (211 mg, 668 μmol) in THF (1 mL) was added and the reaction mixture continued to stir for 1 h. Aqueous NH₄Cl was added to quench the reaction. The reaction was diluted with EtOAc, the layers were separated, and the aqueous layer extracted with EtOAc. The combined organic layers were dried over Na₂SO₄ and the concentrated residue was purified (FCC, SiO₂, 0-70% EtOAc/DCM) to provide tert-butyl ((cis)-3-((3-fluoro-6-(1-methyl-1H-pyrazol-4-yl)pyrazolo[1,5-a]pyrazin-4-yl)oxy)cyclobutyl)(methyl)-carbamate (75.5 mg, 33% yield) as an amorphous solid. LCMS, Rt=0.92 min, m/z 439.2, 317.1. ¹⁹F NMR (376 MHz, CHLOROFORM-d) δ-174.02 (br s, 1F).

### Synthesis of (cis)-3-((3-fluoro-6-(1-methyl-1H-pyrazol-4-yl)pyrazolo[1,5-a]pyrazin-4-yl)oxy)-N-methylcyclobutan-1-amine

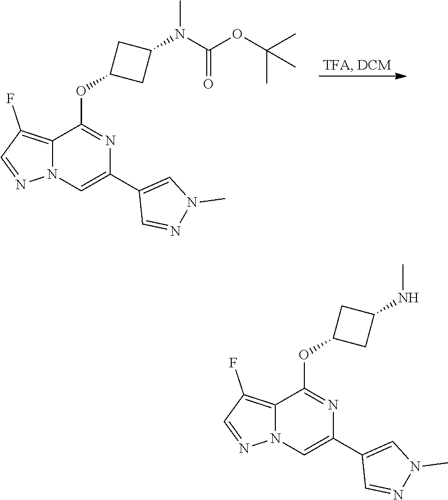

To a solution of tert-butyl ((cis)-3-((3-fluoro-6-(1-methyl-1H-pyrazol-4-yl)pyrazolo[1,5-a]pyrazin-4-yl)oxy)cyclobutyl)(methyl)carbamate (76 mg, 181 μmol) in DCM (1 mL) was added TFA (1.49 g, 13.1 mmol, 1 mL) and the reaction mixture was stirred at RT for 1 h. The crude was concentrated to give (cis)-3-((3-fluoro-6-(1-methyl-1H-pyrazol-4-yl)pyrazolo[1,5-a]pyrazin-4-yl)oxy)-N-methylcyclobutan-1-amine (166 mg, crude, trifluoroacetic acid) as a residue and was used as is for the next step. LCMS: Rt=0.57 min, m/z 317.1.

### Synthesis of N-((cis)-3-((3-fluoro-6-(1-methyl-1H-pyrazol-4-yl)pyrazolo[1,5-a]pyrazin-4-yl)oxy)cyclobutyl)-N-methylacrylamide

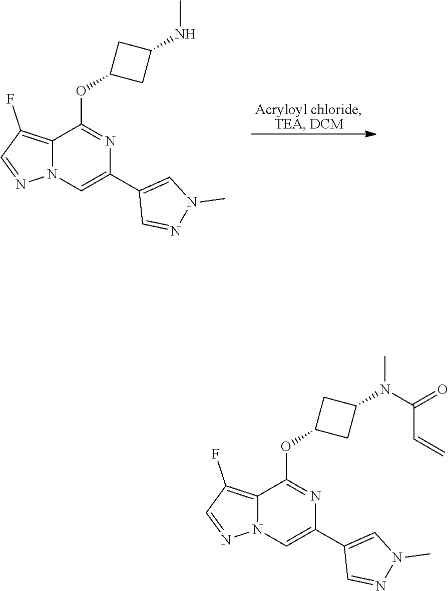 [SMILES: CN[C@H]1C[C@@H](Oc2nc(-c3cnn(C)c3)cn3ncc(F)c23)C1 ; "Acryloyl chloride,TEA, DCM" ; C=CC(=O)N(C)[C@H]1C[C@@H](Oc2nc(-c3cnn(C)c3)cn3ncc(F)c23)C1]

To a solution of (cis)-3-((3-fluoro-6-(1-methyl-1H-pyrazol-4-yl)pyrazolo[1,5-a]pyrazin-4-yl)oxy)-N-methylcyclobutan-1-amine (166 mg, 386 μmol, trifluoroacetic acid) in DCM (5 mL) was added TEA (78 mg, 771 μmol, 108 μL) and the reaction mixture was stirred for 5 min. After cooling to 0° C., acryloyl chloride (42 mg, 463 μmol, 38 μL) was added and stirring continued for 3 min. The reaction was quenched with saturated aqueous NaHCO₃ and extracted with DCM. The organic layer was dried over Na₂SO₄ and the concentrated residue was chromatographed on silica gel (EtOAc/MeOH 0-30%) to give a residue which was further purified with prep HPLC (10-90% H₂O/ACN) to give N-((cis)-3-((3-fluoro-6-(1-methyl-1H-pyrazol-4-yl)pyrazolo[1,5-a]pyrazin-4-yl)oxy)cyclobutyl)-N-methylacrylamide (30.1 mg, 15% yield, 95% purity, trifluoroacetic acid). LCMS: Rt=0.70 min, m/z 371.2 [M+H]⁺, 393.2 [M+Na]⁺. ¹H NMR (400 MHz, CHLOROFORM-d) δ 8.06 (d, J=1.76 Hz, 1H), 7.91 (br s, 1H), 7.84 (br s, 1H), 7.75 (br d, J=3.51 Hz, 1H), 6.58 (br dd, J=10.92, 16.69 Hz, 1H), 6.35 (br d, J=19.07 Hz, 1H), 5.79 (br d, J=10.79 Hz, 1H), 5.17-5.25 (m, 1H), 4.82 (br s, 1H), 4.03 (s, 3H), 3.11 (br s, 3H), 3.00 (br s, 2H), 2.59 (br s, 1H), 2.43 (br s, 1H). ¹⁹F NMR (376 MHz, CHLOROFORM-d) δ-75.97 (s, 3F), −173.35 (br s, 1F).

### Example 64: N-((trans)-3-((3-(difluoromethyl)-6-(1-methyl-1H-pyrazol-4-yl)pyrazolo[1,4-a]pyrazin-4-yl)oxy)cyclobutyl-N-methacrylamide

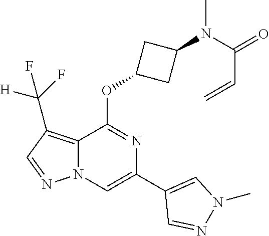 [SMILES: C=CC(=O)N(C)[C@H]1C[C@H](Oc2nc(-c3cnn(C)c3)cn3ncc(C(F)F)c23)C1]

### Synthesis of tert-butyl methyl((trans)-3-((6-(1-methyl-1H-pyrazol-4-yl)-3-vinylpyrazolo[1,5-a]pyrazin-4-yl)oxy)cyclobutyl)carbamate

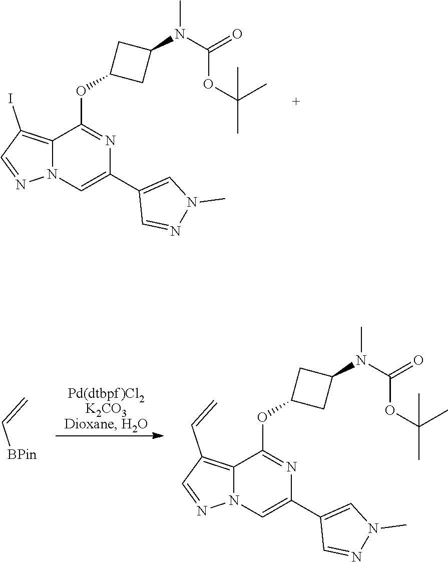

A microwave vial was charged with tert-butyl ((trans)-3-((3-iodo-6-(1-methyl-1H-pyrazol-4-yl)pyrazolo[1,5-a]pyrazin-4-yl)oxy)cyclobutyl)(methyl)carbamate (135 mg, 257 μmol), 4,4,5,5-tetramethyl-2-vinyl-1,3,2-dioxaborolane (79 mg, 515 μmol, 87 μL), di-tert-butyl(cyclopentyl)phosphane; dichloropalladium; iron (34 mg, 51 μmol) and potassium carbonate (100 mg, 721 μmol) and set under N₂. The vial was capped and dioxane (1.2 mL) and water (0.3 mL) were added via syringe and the red mixture was set under N₂ again (2 cycles). After stirring for 5 minutes at RT, the mixture was heated to 90° C. and was stirred at that temperature for 5 h. After cooling to RT, the mixture was diluted with EtOAc and filtered. The filtrate was evaporated in vacuo and the residual material purified on a 10 g Si-SPE: Rf=0.27 in heptanes/EtOAc=5/1 to give tert-butyl methyl((trans)-3-((6-(1-methyl-1H-pyrazol-4-yl)-3-vinylpyrazolo[1,5-a]pyrazin-4-yl)oxy)cyclobutyl)carbamate (110 mg, 91% yield, 90% purity) as a yellow gum.

### Synthesis of tert-butyl ((trans)-3-((3-formyl-6-(1-methyl-1H-pyrazol-4-yl)pyrazolo[1,5-a]pyrazin-4-yl)oxy)cyclobutyl)(methyl)carbamate

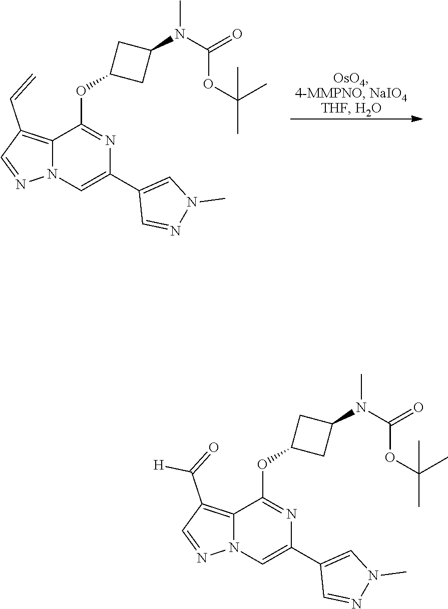 [SMILES: C=Cc1cnn2cc(-c3cnn(C)c3)nc(O[C@H]3C[C@H](N(C)C(=O)OC(C)(C)C)C3)c12 ; "OsO4,4-MMPNO, NaIO4THF, H2O" ; Cn1cc(-c2cn3ncc(C=O)c3c(O[C@H]3C[C@H](N(C)C(=O)OC(C)(C)C)C3)n2)cn1]

A vial was charged with tert-butyl methyl((trans)-3-((6-(1-methyl-1H-pyrazol-4-yl)-3-vinylpyrazolo[1,5-a]pyrazin-4-yl)oxy)cyclobutyl)carbamate (110 mg, 259 μmol) and water (0.5 mL) and THF (1.5 mL) and cooled in an ice bath. Then, osmium tetroxide (132 mg, 26 μmol, 5% purity; resin-bound) and 4-methyl-4-oxido-morpholin-4-ium (30 mg, 259 μmol) were added and stirring in the ice bath was continued for 1 h. Then, sodium (meta)periodate (111 mg, 518 μmol) was added and stirring in the water bath was continued as the reaction mixture warmed to RT overnight. Then saturated Na₂S₂O₃ was added followed by DCM. The mixture was filtered, and the organic phase was separated, dried, and evaporated in vacuo to give a dark green sticky gum. This material was carried forward without further purification. ESI-MS (M+Na)⁺: 449.4.

### Synthesis of tert-butyl ((trans)-3-((3-(difluoromethyl)-6-(1-methyl-1H-pyrazol-4-yl)pyrazolo[1,5-a]pyrazin-4-yl)oxy)cyclobutyl)(methyl)carbamate

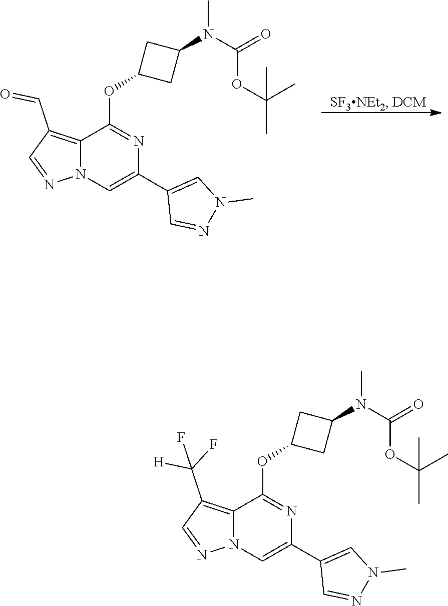

A vial was charged with tert-butyl ((trans)-3-((3-formyl-6-(1-methyl-1H-pyrazol-4-yl)pyrazolo[1,5-a]pyrazin-4-yl)oxy)cyclobutyl)(methyl)carbamate (90 mg, 211 μmol) and DCM (2 mL), set under N₂, and cooled in an ice bath. Then, N-ethyl-N-(trifluoro-sulfanyl)ethanamine (68 mg, 422 mol, 56 μL) was added dropwise with stirring. Stirring was continued overnight during which the mixture gradually warmed to rt. The mixture was diluted with DCM and silica gel was added. The volatiles are evaporated in vacuo and the residual material was purified on a 5 g Si-SPE: Rf=0.5 in EtOAc to give tert-butyl ((trans)-3-((3-(difluoromethyl)-6-(1-methyl-1H-pyrazol-4-yl)pyrazolo[1,5-a]pyrazin-4-yl)oxy)cyclobutyl)(methyl)carbamate (45 mg, 43% yield, 90% purity) as a pale-yellow sticky gum. ESI-MS (M+H)⁺: 449.5.

### Synthesis of (trans)-3-((3-(difluoromethyl)-6-(1-methyl-1H-pyrazol-4-yl)pyrazolo[1,5-a]pyrazin-4-yl)oxy)-N-methylcyclobutan-1-amine

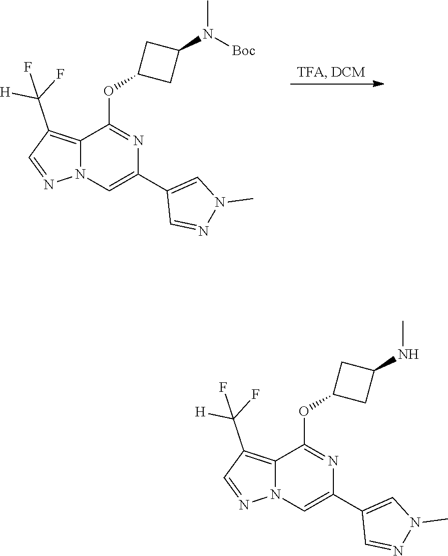

To a solution of tert-butyl ((trans)-3-((3-(difluoromethyl)-6-(1-methyl-1H-pyrazol-4-yl)pyrazolo[1,5-a]pyrazin-4-yl)oxy)cyclobutyl)(methyl)carbamate (40 mg, 89 μmol) in DCM (2 mL) was added TFA (102 mg, 892 μmol, 68 μL) with stirring at rt. After stirring overnight, the mixture was diluted with MeOH and purified on a 2 g SCX column where the product eluted with 2 M NH₃-MeOH to give, after removal of the volatiles, (trans)-3-((3-(difluoromethyl)-6-(1-methyl-1H-pyrazol-4-yl)pyrazolo[1,5-a]pyrazin-4-yl)oxy)-N-methylcyclobutan-1-amine (28 mg, 81% yield, 90% purity) as a yellow gum.

### Synthesis of N-((trans)-3-((3-(difluoromethyl)-6-(1-methyl-1H-pyrazol-4-yl)pyrazolo[1,5-a]pyrazin-4-yl)oxy)cyclobutyl)-N-methylacrylamide

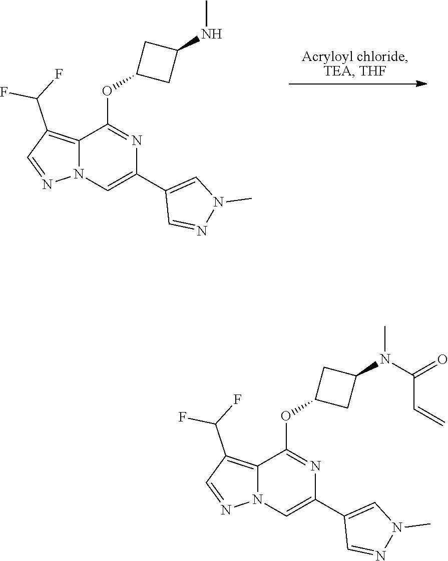 [SMILES: CN[C@H]1C[C@H](Oc2nc(-c3cnn(C)c3)cn3ncc(C(F)F)c23)C1 ; "Acryloyl chloride,TEA, THF" ; C=CC(=O)N(C)[C@H]1C[C@H](Oc2nc(-c3cnn(C)c3)cn3ncc(C(F)F)c23)C1]

A vial was charged with (trans)-3-((3-(difluoromethyl)-6-(1-methyl-1H-pyrazol-4-yl)pyrazolo[1,5-a]pyrazin-4-yl)oxy)-N-methylcyclobutan-1-amine (28 mg, 80 μmol) and THF (1 mL). Then, acryloyl chloride (7.2 mg, 80 μmol) was added upon which precipitation occurred instantaneously. Then, TEA (12 mg, 121 μmol, 17 μL) was added and stirring at RT was continued for 1 h. The volatiles were evaporated under vacuum and the residual material was purified on basic prep-HPLC (Waters XSelect CSH C18, 5 μm, 30 mm×50 mm column with mobile phase H₂O (A) and MeCN (B) and a gradient of 5-60% B (0.2% NH₄OH final v/v % modifier) with flow rate at 60 m/min) to give, after lyophilization of the HPLC fractions, N-((trans)-3-((3-(difluoromethyl)-6-(1-methyl-1H-pyrazol-4-yl)pyrazolo[1,5-a]pyrazin-4-yl)oxy)cyclobutyl)-N-methylacrylamide (6.8 mg, 19% yield, 90% purity) as a white solid. ESI-MS (M+H)⁺: 403.4. ¹H NMR (CHLOROFORM-d, 400 MHz): δ=10.39-10.44 (m, 1H), 8.45-8.47 (m, 1H), 8.30 (s, 1H), 7.9 (m, 1H), 7.82 (m, 1H), 6.50-6.66 (m, 1H), 6.31 (br d, J=15.8 Hz, 1H), 5.72 (br d, J=10.3 Hz, 1H), 5.63 (br s, 1H), 5.07 (br s, 1H), 4.01 (s, 3H), 3.12 (s, 3H), 2.80-2.90 (m, 2H), 2.70-2.78 (m, 1H), 2.64-2.84 (m, 2H).

### Example 65: 1-[(4R)-4-[7-(1-methylpyrazol-4-yl)imidazo[1,2-a]pyridin-5-yl]oxyazepan-1-yl]prop-2-en-1-one

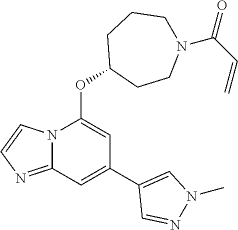 [SMILES: C=CC(=O)N1CCC[C@@H](Oc2cc(-c3cnn(C)c3)cc3nccn23)CC1]

### Synthesis of tert-butyl (4R)-4-(7-iodoimidazo[1,2-a]pyridin-5-yl)oxyazepane-1-carboxylate

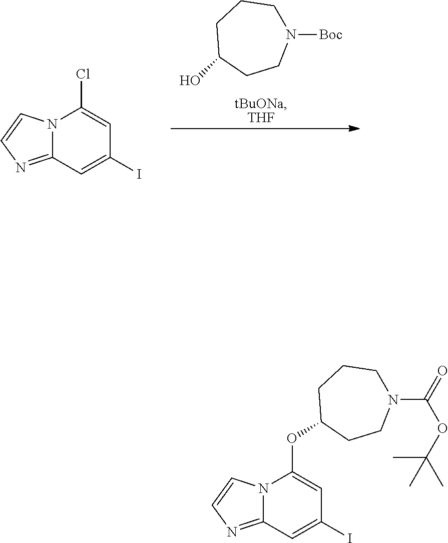

A vial containing 5-chloro-7-iodo-imidazo[1,2-a]pyridine (155 mg, 557 μmol) in anhydrous THF (2 mL) was cooled in an ice water bath, then sodium tert-butoxide (93 mg, 970 μmol) was added carefully in portions to the cold mixture. After 15 minutes, tert-butyl (4R)-4-hydroxyazepane-1-carboxylate (142 mg, 661 μmol) was added carefully in portions to the cold heterogeneous mixture. Upon complete addition of 5-chloro-7-iodo-imidazo[1,2-a]pyridine, the mixture was allowed to warm to 23° C. and monitored with LCMS. After 19 hours, the reaction was carefully quenched with slow addition of water then the biphasic mixture was extracted three times with ethyl acetate. The organics were pooled then dried over anhydrous sodium sulfate. After filtration and concentration under reduced pressure, the residue was loaded onto a silica gel column and purified with (20-65% ethyl acetate in heptanes). The desired fractions were pooled then concentrated under reduced pressure to afford a light yellow oil as tert-butyl (4R)-4-(7-iodoimidazo[1,2-a]pyridin-5-yl)oxyazepane-1-carboxylate (119 mg, 47% yield) that was used without further purification. LCMS m/z=458.0 (M+H)⁺.

### Synthesis of tert-butyl (4R)-4-[7-(1-methylpyrazol-4-yl)imidazo[1,2-a]pyridin-5-yl]oxyazepane-1-carboxylate

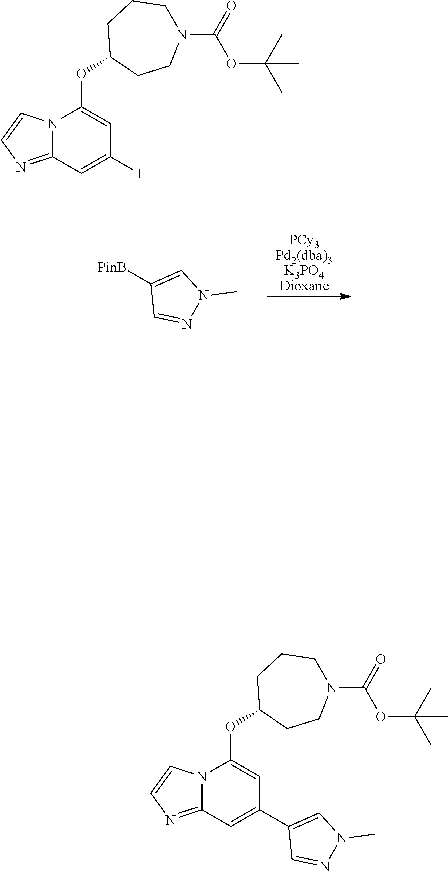

A vial containing tert-butyl (4R)-4-(7-iodoimidazo[1,2-a]pyridin-5-yl)oxyazepane-1-carboxylate (119 mg, 260 μmol), 1-methyl-4-(4,4,5,5-tetramethyl-1,3,2-dioxaborolan-2-yl)pyrazole (111 mg, 534 μmol), tricyclohexylphosphane (19.4 mg, 69 μmol), tris(dibenzylideneacetone) dipalladium (25.4 mg, 28 μmol), and potassium phosphate tribasic(1 M, 0.8 mL) in dioxane (1 mL) was degassed then backfilled with nitrogen. After evacuation and backfilling with nitrogen (×3), the reaction was heated to 90° C. and monitored with LCMS. After 2 hours, the reaction was carefully quenched with slow addition of water. The biphasic mixture was extracted three times with ethyl acetate then dried over anhydrous sodium sulfate. After filtration and concentration under reduced pressure, the residue was loaded onto a silica gel column and purified with (20-100% [3:1 ethyl acetate: ethanol] in heptanes). The desired fractions were pooled then concentrated under reduced pressure to afford a yellow film as tert-butyl (4R)-4-[7-(1-methylpyrazol-4-yl)imidazo[1,2-a]pyridin-5-yl]oxyazepane-1-carboxylate (75.9 mg, 71% yield) that was used without further purification. LCMS m/z=412.1 (M+H)⁺.

### Synthesis of 5-[(4R)-azepan-4-yl]oxy-7-(1-methylpyrazol-4-yl)imidazo[1,2-a]pyridine

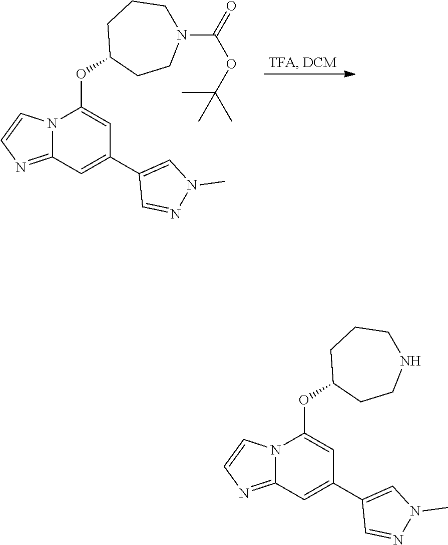

A vial containing tert-butyl (4R)-4-[7-(1-methylpyrazol-4-yl)imidazo[1,2-a]pyridin-5-yl]oxyazepane-1-carboxylate (76 mg, 184 μmol) in anhydrous dichloromethane (0.5 mL) was cooled in an ice water bath, then trifluoroacetic acid (149 mg, 1.31 mmol, 0.1 mL) was added carefully dropwise to the cold mixture. Upon complete addition of TFA, the mixture was allowed to warm to 23° C. and monitored with LCMS. After 19 hours, the reaction was carefully concentrated under reduced pressure to afford a light yellow film as 5-[(4R)-azepan-4-yl]oxy-7-(1-methylpyrazol-4-yl)imidazo[1,2-a]pyridine (crude, trifluoroacetic acid) that was used without purification. LCMS m/z=312.0 (M+H)⁺.

### Synthesis of 1-[(4R)-4-[7-(1-methylpyrazol-4-yl)imidazo[1,2-a]pyridin-5-yl]oxyazepan-1-yl]prop-2-en-1-one

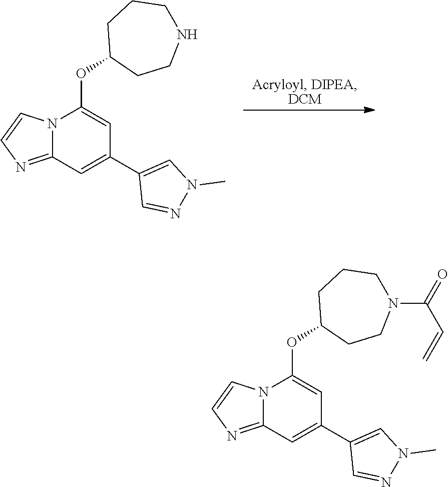 [SMILES: Cn1cc(-c2cc(O[C@@H]3CCCNCC3)n3ccnc3c2)cn1 ; "Acryloyl, DIPEA,DCM" ; C=CC(=O)N1CCC[C@@H](Oc2cc(-c3cnn(C)c3)cc3nccn23)CC1]

To a vial containing 5-[(4R)-azepan-4-yl]oxy-7-(1-methylpyrazol-4-yl)imidazo[1,2-a]pyridine (79 mg, 254 μmol, trifluoroacetic acid) in anhydrous THF (1 mL) was added Hunigs base (445 mg, 3.44 mmol, 0.6 mL) carefully at −25° C. After 4 minutes, acryloyl chloride (45 mg, 492 μmol, 0.04 mL) was added carefully dropwise to the cold homogeneous solution. Upon complete addition of acryloyl chloride, the reaction was allowed to warm to 23° C. and monitored with LCMS and TLC. After 3 minutes, the reaction was carefully quenched with slow addition of saturated aqueous sodium bicarbonate solution. The biphasic mixture was loaded onto a silica gel column and purified with (40-100% [3:1 ethyl acetate ethanol] in heptane, then flushed with 20% methanol in dichloromethane). The desired fractions were pooled then concentrated under reduced pressure to afford 67 mg of a colorless film that was further purified by mass directed reverse phase HPLC (Waters XSelect CSH C18, 5 μm, 19 mm×100 mm column with mobile phase H₂O (A) and MeCN (B) and a gradient of 5-50% B (0.2% NH₄OH final v/v % modifier) with flow rate at 30 mL/min.). Fractions containing the desired product were concentrated to afford a colorless film as 1-[(4R)-4-[7-(1-methylpyrazol-4-yl)imidazo[1,2-a]pyridin-5-yl]oxyazepan-1-yl]prop-2-en-1-one (1.6 mg, 2% yield). ¹H NMR (500 MHz, DMSO-d6) δ=8.52-8.12 (m, 2H), 8.02-7.64 (m, 2H), 7.47 (s, 1H), 6.96-6.77 (m, 2H), 6.23-6.13 (m, 1H), 5.75-5.65 (m, 1H), 5.28-5.16 (m, 1H), 3.96-3.85 (m, 3H), 3.84-3.46 (m, 6H), 2.12-2.07 (m, 1H), 1.99-1.91 (m, 2H), 1.83-1.72 (m, 1H). LCMS: m/z=366.1 (M+H)⁺.

### Example 66: 1-[(4R)-4-[6-(1-methylpyrazol-4-yl)pyrazolo[1,5-a]pyridin-4-yl]oxyazepan-1-yl]prop-2-en-1-one

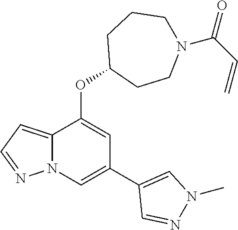 [SMILES: C=CC(=O)N1CCC[C@@H](Oc2cc(-c3cnn(C)c3)cn3nccc23)CC1]

### Synthesis of tert-butyl (4R)-4-(6-bromopyrazolo[1,5-a]pyridin-4-yl)oxyazepane-1-carboxylate

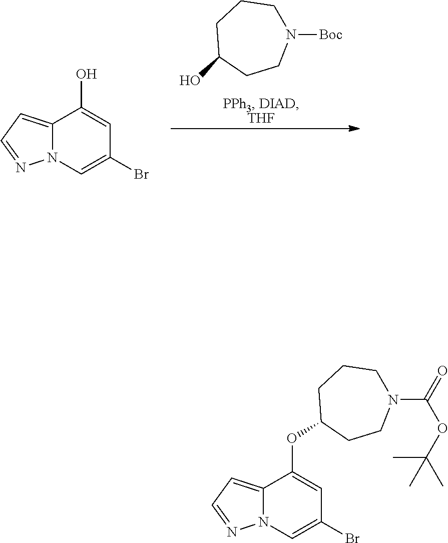

To a solution of tert-butyl (4S)-4-hydroxyazepane-1-carboxylate (303 mg, 1.41 mmol), 6-bromopyrazolo[1,5-a]pyridin-4-ol (300 mg, 1.41 mmol), and triphenylphosphine (554 mg, 2.11 mmol) in THF (5 mL) was added DIAD (342 mg, 1.69 mmol, 333 μL) and stirred at RT for 16 h. The concentrated crude was chromatographed on silica gel (heptanes/EtOAc 0-60%) to give tert-butyl (4R)-4-(6-bromopyrazolo[1,5-a]pyridin-4-yl)oxyazepane-1-carboxylate (255 mg, 42% yield, 95% purity) as an colorless oil. LCMS: Rt=1.00 min, m/z 356.1, 412.1 (M+H)⁺.

### Synthesis of tert-butyl (4R)-4-[6-(1-methylpyrazol-4-yl)pyrazolo[1,5-a]pyridin-4-yl]oxyazepane-1-carboxylate

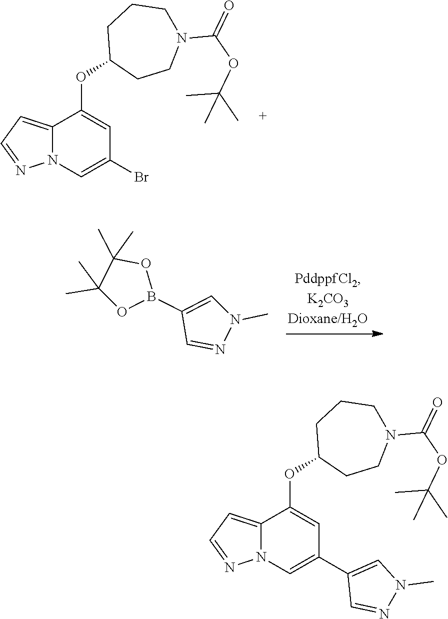

A solution of 1-methyl-4-(4,4,5,5-tetramethyl-1,3,2-dioxaborolan-2-yl)pyrazole (194 mg, 932 μmol), Pd(dppf)Cl₂ DCM (51 mg, 62 μmol), and K₂CO₃ (258 mg, 1.86 mmol) in dioxane (3 mL) and water (0.5 mL) was degassed and heated to 95° C. for 16 h. After cooling to RT, the mixture was filtrated though Celite and concentrated. The residue was chromatographed on silica gel (heptanes/EtOAc 0-100%) to give tert-butyl (4R)-4-[6-(1-methylpyrazol-4-yl)pyrazolo[1,5-a]pyridin-4-yl]oxyazepane-1-carboxylate (192 mg, 71% yield, 95% purity) as a yellow gel. LCMS: Rt=0.87 min, m/z 412.3 (M+H)⁺.

### Synthesis of 4-[(4R)-azepan-4-yl]oxy-6-(1-methylpyrazol-4-yl)pyrazolo[1,5-a]pyridine

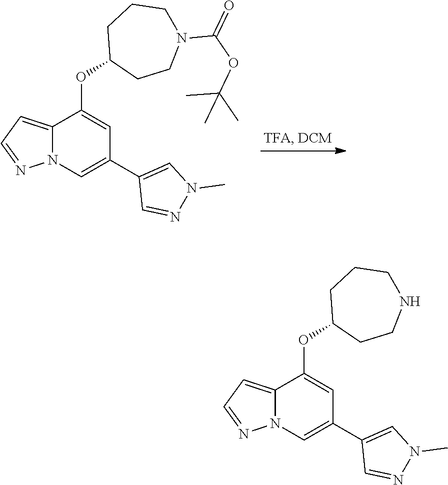 [SMILES: Cn1cc(-c2cc(O[C@@H]3CCCN(C(=O)OC(C)(C)C)CC3)c3ccnn3c2)cn1 ; Cn1cc(-c2cc(O[C@@H]3CCCNCC3)c3ccnn3c2)cn1]

To a solution of tert-butyl (4R)-4-[6-(1-methylpyrazol-4-yl)pyrazolo[1,5-a]pyridin-4-yl]oxyazepane-1-carboxylate (192 mg, 467 μmol) in DCM (1 mL) was added TFA (1.49 g, 13.1 mmol, 1 mL) and the reaction mixture was stirred at RT for 1 h. The crude was concentrated to give 4-[(4R)-azepan-4-yl]oxy-6-(1-methylpyrazol-4-yl)pyrazolo[1,5-a]pyridine (345 mg, crude, trifluoroacetic acid) was used as is for the next step. LCMS: Rt=0.50 min, m/z 312.1 (M+H)⁺.

### Synthesis of 1-[(4R)-4-[6-(1-methylpyrazol-4-yl)pyrazolo[1,5-a]pyridin-4-yl]oxyazepan-1-yl]prop-2-en-1-one

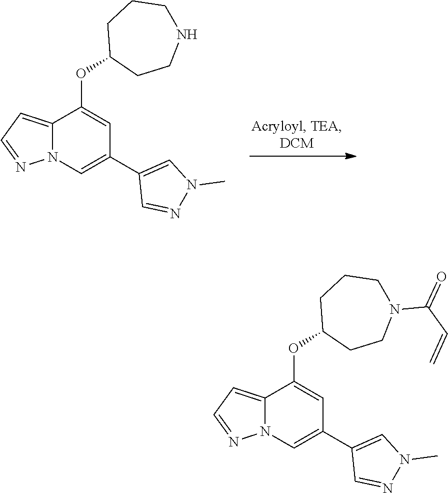 [SMILES: Cn1cc(-c2cc(O[C@@H]3CCCNCC3)c3ccnn3c2)cn1 ; C=CC(=O)N1CCC[C@@H](Oc2cc(-c3cnn(C)c3)cn3nccc23)CC1]

To a solution of 4-[(4R)-azepan-4-yl]oxy-6-(1-methylpyrazol-4-yl)pyrazolo[1,5-a]pyridine (345 mg, 811 μmol, trifluoroacetic acid) in DCM (10 mL) was added TEA (164 mg, 1.62 mmol, 226 μL) and the reaction mixture was stirred for 5 min. After cooling to 0° C., acryloyl chloride (88 mg, 973 μmol, 79 μL) was added and the reaction mixture was stirred for 3 min. The reaction was quenched with saturated aqueous NaHCO₃ and extracted with DCM. The organic layer was dried over Na₂SO₄ and the concentrated residue was chromatographed on silica gel (EtOAc/MeOH 0-30%) to give 1-[(4R)-4-[6-(1-methylpyrazol-4-yl)pyrazolo[1,5-a]pyridin-4-yl]oxyazepan-1-yl]prop-2-en-1-one (112.1 mg, 36% yield, 95% purity). LCMS: Rt=0.62 min, m/z 366.2 (M+H)⁺. ¹H NMR (400 MHz, CHLOROFORM-d) δ 8.25 (s, 1H), 7.84 (t, J=1.76 Hz, 1H), 7.70 (d, J=3.26 Hz, 1H), 7.59 (d, J=6.02 Hz, 1H), 6.55-6.66 (m, 2H), 6.44 (d, J=8.28 Hz, 1H), 6.31-6.41 (m, 1H), 5.68-5.75 (m, 1H), 4.72 (br s, 1H), 3.95 (s, 3H), 3.63-3.80 (m, 2H), 3.45-3.61 (m, 2H), 2.07-2.25 (m, 4H), 1.88-1.99 (m, 1H), 1.69-1.86 (m, 1H).

### Example 67: 1-[(4R)-4-[2-amino-6-(1-methylpyrazol-4-yl)pyrazolo[1,5-a]pyrazin-4-yl]oxyazepan-1-yl]prop-2-en-1-one

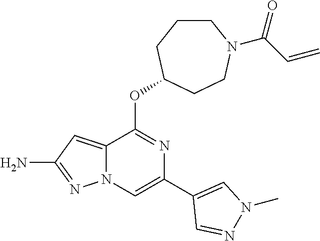 [SMILES: C=CC(=O)N1CCC[C@@H](Oc2nc(-c3cnn(C)c3)cn3nc(N)cc23)CC1]

### Synthesis of ethyl 4-chloro-6-(1-methyl-1H-pyrazol-4-yl)pyrazolo[1,5-a]pyrazine-2-carboxylate

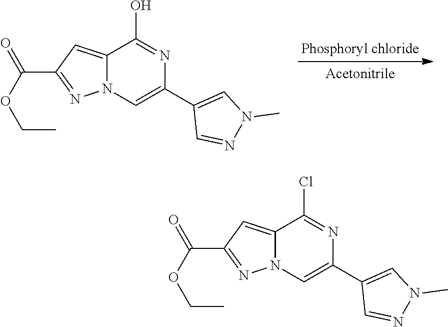 [SMILES: CCOC(=O)c1cc2c(O)nc(-c3cnn(C)c3)cn2n1 ; CCOC(=O)c1cc2c(Cl)nc(-c3cnn(C)c3)cn2n1]

To a suspension of ethyl 4-hydroxy-6-(1-methylpyrazol-4-yl)pyrazolo[1,5-a]pyrazine-2-carboxylate (130 mg, 454 μmol) in dry acetonitrile (2 mL) under nitrogen was added phosphoryl chloride (1.11 g, 7.27 mmol, 677 μL) dropwise. The resulting mixture was heated to 80° C. for 17 h. After cooling to RT, the reaction mixture was diluted with EtOAc and quenched carefully with saturated aqueous sodium bicarbonate solution. The layers were separated, and the organic layer was washed sequentially with saturated bicarbonate solution (2×) and brine. The organic phase was dried (MgSO₄), filtered, and concentrated in vacuo. Ethyl 4-chloro-6-(1-methylpyrazol-4-yl)pyrazolo[1,5-a]pyrazine-2-carboxylate was obtained as a white solid, which was used directly. Quantitative yield assumed. ¹H NMR (500 MHz, CHLOROFORM-d) δ ppm 8.50 (d, J=1.2 Hz, 1H), 7.94 (s, 1H), 7.90 (s, 1H), 7.37-7.42 (m, 1H), 4.50 (q, J=6.9 Hz, 2H), 3.97-4.01 (m, 3H), 1.46 (t, J=7.0 Hz, 3H). LCMS: m/z=306.3 [M+H]+.

### Synthesis of (R)-1-(tert-butoxycarbonyl)azepan-4-yl 4-(((R)-1-(tert-butoxycarbonyl)azepan-4-yl)oxy)-6-(1-methyl-1H-pyrazol-4-yl)pyrazolo[1,5-a]pyrazine-2-carboxylate

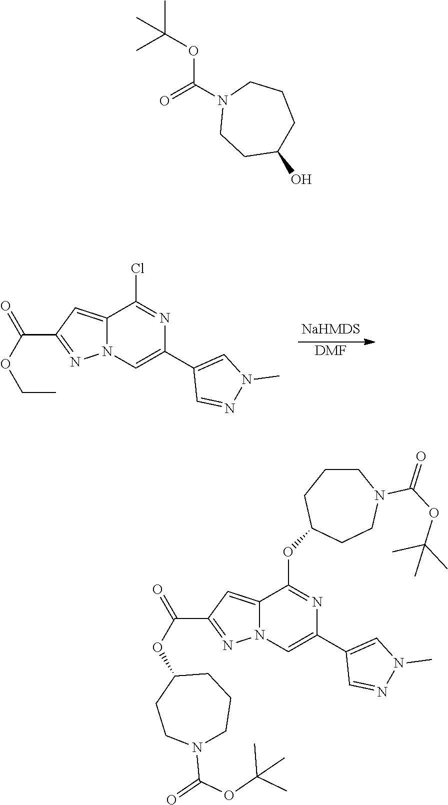

To a solution of tert-butyl (4R)-4-hydroxyazepane-1-carboxylate (232 mg, 1.08 mmol) in dry DMF (2 mL) under nitrogen was added NaHMDS (1 M, 1.08 mL) at 20° C. The mixture was stirred at 20° C. for 15 min. A solution of ethyl 4-chloro-6-(1-methylpyrazol-4-yl)pyrazolo[1,5-a]pyrazine-2-carboxylate (150 mg, 491 μmol) in dry DMF (2 mL) was added and the resulting mixture was stirred at RT for 2 h, quenched with H₂O (1 mL) and diluted with EtOAc. The organic layer was washed with brine (3×), dried over MgSO₄, filtered, and concentrated under vacuum. The residue was purified by silica gel chromatography (0-100% EtOAc in heptanes) to give [(4R)-1-tert-butoxycarbonylazepan-4-yl] 4-[(4R)-1-tert-butoxycarbonylazepan-4-yl]oxy-6-(1-methylpyrazol-4-yl)pyrazolo[1,5-a]pyrazine-2-carboxylate (136 mg, 42% yield) as a pale yellow foam. ¹H NMR (500 MHz, CHLOROFORM-d) δ ppm 8.23 (s, 1H), 7.76-7.90 (m, 2H), 5.55 (br s, 1H), 5.22-5.32 (m, 1H), 3.98 (s, 3H), 3.58-3.72 (m, 2H), 3.30-3.58 (m, 6H), 2.06-2.25 (m, 4H), 1.89-2.06 (m, 6H), 1.69-1.84 (m, 2H), 1.51 (s, 9H), 1.49 (s, 9H). LCMS: m/z=654.7 [M+H]+.

### Synthesis of (R)-4-((1-(tert-butoxycarbonyl)azepan-4-yl)oxy)-6-(1-methyl-1H-pyrazol-4-yl)pyrazolo[1,5-a]pyrazine-2-carboxylic acid

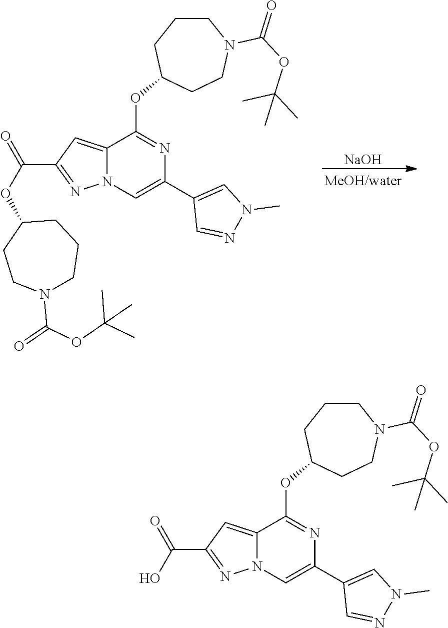 [SMILES: Cn1cc(-c2cn3nc(C(=O)O[C@@H]4CCCN(C(=O)OC(C)(C)C)CC4)cc3c(O[C@@H]3CCCN(C(=O)OC(C)(C)C)CC3)n2)cn1 ; "NaOH" ; Cn1cc(-c2cn3nc(C(=O)O)cc3c(O[C@@H]3CCCN(C(=O)OC(C)(C)C)CC3)n2)cn1]

To a solution of [(4R)-1-tert-butoxycarbonylazepan-4-yl] 4-[(4R)-1-tert-butoxycarbonylazepan-4-yl]oxy-6-(1-methylpyrazol-4-yl)pyrazolo[1,5-a]pyrazine-2-carboxylate (41 mg, 63 μmol) in methanol (400 μL) was added sodium hydroxide solution (2 M, 94 μL) and the resulting mixture was stirred at RT for 30 min. The reaction mixture was quenched with HCl solution (1 M, 188 μL) and diluted with EtOAc and water. The layers were separated, the organic one was washed with water and brine, dried (MgSO₄), filtered and concentrated in vacuo. 4-[(4R)-1-tert-butoxycarbonylazepan-4-yl]oxy-6-(1-methylpyrazol-4-yl)pyrazolo[1,5-a]pyrazine-2-carboxylic acid was obtained as a white solid and which used directly. Quantitative yield assumed. LCMS: m/z=457.4 [M+H]+.

### Synthesis of tert-butyl (R)-4-((2-((tert-butoxycarbonyl)amino)-6-(1-methyl-1H-pyrazol-4-yl)pyrazolo[1,5-a]pyrazin-4-yl)oxy)azepane-1-carboxylate

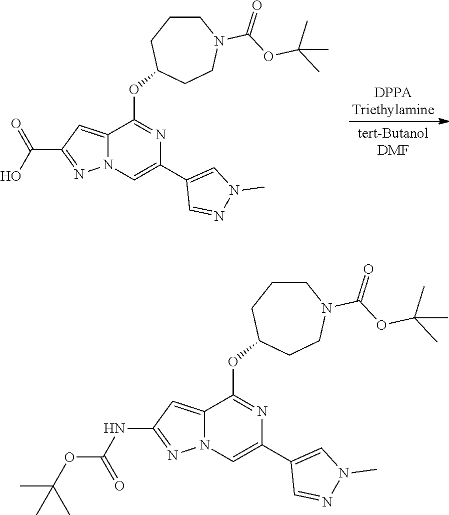

To a solution of 4-[(4R)-1-tert-butoxycarbonylazepan-4-yl]oxy-6-(1-methylpyrazol-4-yl)pyrazolo[1,5-a]pyrazine-2-carboxylic acid (110 mg, 241 μmol) in dry DMF (1 mL) and tert-butanol (0.5 mL) was added triethylamine (37 mg, 361 μmol, 50 μL) followed by DPPA (99 mg, 361 μmol, 78 μL) dropwise at rt. The resulting mixture was stirred at 80° C. for 17 h, cooled to RT, diluted with EtOAc, and washed with water and brine (3×). The organic layer was dried (MgSO₄), filtered, and concentrated in vacuo. The residue was purified by silica gel chromatography (0-5% MeOH in DCM). tert-Butyl (4R)-4-[2-(tert-butoxycarbonylamino)-6-(1-methylpyrazol-4-yl)pyrazolo[1,5-a]pyrazin-4-yl]oxyazepane-1-carboxylate (38 mg, 30% yield) was obtained as an oil. ¹H NMR (500 MHz, CHLOROFORM-d) δ ppm 7.99 (s, 1H), 7.72-7.84 (m, 2H), 7.33 (br s, 1H), 6.82-7.05 (bs, 1H), 5.47-5.55 (m, 1H), 3.96 (s, 3H), 3.58-3.76 (m, 2H), 3.45-3.56 (m, 2H), 1.88-2.03 (m, 6H), 1.50 (s, 9H), 1.46 (s, 9H). LCMS: m/z=528.3 [M+H]+.

### Synthesis of (R)-4-(azepan-4-yloxy)-6-(1-methyl-1H-pyrazol-4-yl)pyrazolo[1,5-a]pyrazin-2-amine bishydrochloride

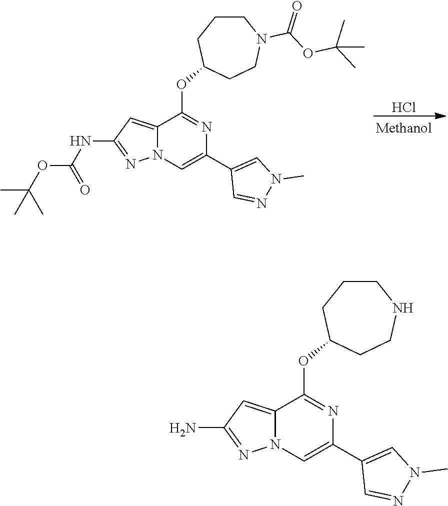 [SMILES: Cn1cc(-c2cn3nc(NC(=O)OC(C)(C)C)cc3c(O[C@@H]3CCCN(C(=O)OC(C)(C)C)CC3)n2)cn1 ; Cl ; Cn1cc(-c2cn3nc(N)cc3c(O[C@@H]3CCCNCC3)n2)cn1]

To a solution of tert-butyl (4R)-4-[2-(tert-butoxycarbonylamino)-6-(1-methylpyrazol-4-yl)pyrazolo[1,5-a]pyrazin-4-yl]oxyazepane-1-carboxylate (38 mg, 72 μmol) in methanol (0.6 mL) was added HCl (4 M in dioxane, 360 μL). The resulting solution was stirred at RT for 2 h and was concentrated in vacuo. 4-[(4R)-azepan-4-yl]oxy-6-(1-methylpyrazol-4-yl)pyrazolo[1,5-a]pyrazin-2-amine (2Hydrochloride) was obtained as an off-white solid, which was used directly. Quantitative yield assumed. LCMS: m/z=328.1 [M+H]+.

### Synthesis of 1-[(4R)-4-[2-amino-6-(1-methylpyrazol-4-yl)pyrazolo[1,5-a]pyrazin-4-yl]oxyazepan-1-yl]prop-2-en-1-one

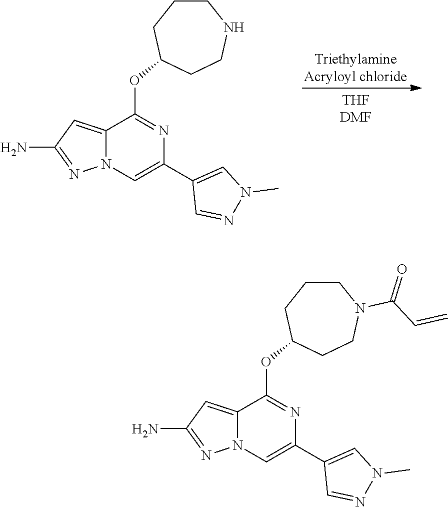 [SMILES: Cn1cc(-c2cn3nc(N)cc3c(O[C@@H]3CCCNCC3)n2)cn1 ; "TriethylamineAcryloyl chloride" ; C=CC(=O)N1CCC[C@@H](Oc2nc(-c3cnn(C)c3)cn3nc(N)cc23)CC1]

To a suspension of crude 4-[(4R)-azepan-4-yl]oxy-6-(1-methylpyrazol-4-yl)pyrazolo[1,5-a]pyrazin-2-amine (28 mg, 70 μmol, 2Hydrochloride) in dry THF (1 mL) and dry DMF (0.5 mL) under nitrogen was added triethylamine (21 mg, 210 μmol, 29 μL), and the resulting suspension was cooled to 0° C. Acryloyl chloride (6 mg, 70 μmol, 6 μL) was added dropwise and the resulting mixture was stirred at 0° C. for 30 min. After quenching with saturated sodium bicarbonate solution and diluting with EtOAc, the layers were separated. The organic layer was washed with brine (3×), dried (MgSO₄), filtered, and concentrated in vacuo. The residue was purified by silica gel chromatography and the desired product was further purified by preparative TLC (96:4 DCM/MeOH). 1-[(4R)-4-[2-amino-6-(1-methylpyrazol-4-yl)pyrazolo[1,5-a]pyrazin-4-yl]oxyazepan-1-yl]prop-2-en-1-one (4.2 mg, 15% yield, 95% purity) was obtained as an off-white solid. ¹H NMR (500 MHz, CHLOROFORM-d) δ ppm 7.93 (s, 1H), 7.79 (s, 1H), 7.68-7.76 (m, 1H), 6.58-6.67 (m, 1H), 6.36-6.43 (m, 1H), 5.94 (d, J=4.3 Hz, 1H), 5.72 (dd, J=10.4, 2.4 Hz, 1H), 5.49-5.60 (m, 1H), 4.02 (br s, 2H), 3.96 (s, 3H), 3.80-3.94 (m, 1H), 3.55-3.77 (m, 3H), 2.11-2.32 (m, 3H), 1.93-2.11 (m, 2H), 1.77-1.90 (m, 1H). LCMS: m/z=382.1 [M+H]+.

### Example 68: N-((cis)-3-((2-amino-6-(1-methyl-1H-pyrazol-4-yl)pyrazolo[1,5-a]pyrazin-4-yl)oxy)cyclobutyl)-N-methylacrylamide

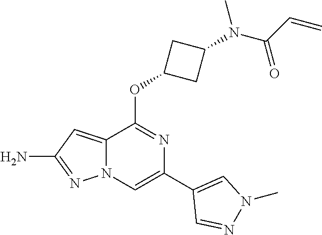 [SMILES: C=CC(=O)N(C)[C@H]1C[C@@H](Oc2nc(-c3cnn(C)c3)cn3nc(N)cc23)C1]

### Synthesis of (cis)-3-((tert-butoxycarbonyl)(methyl)amino)cyclobutyl 4-((cis)-3-((tert-butoxycarbonyl)(methyl)amino)cyclobutoxy)-6-(1-methyl-1H-pyrazol-4-yl)pyrazolo[1,5-a]pyrazine-2-carboxylate

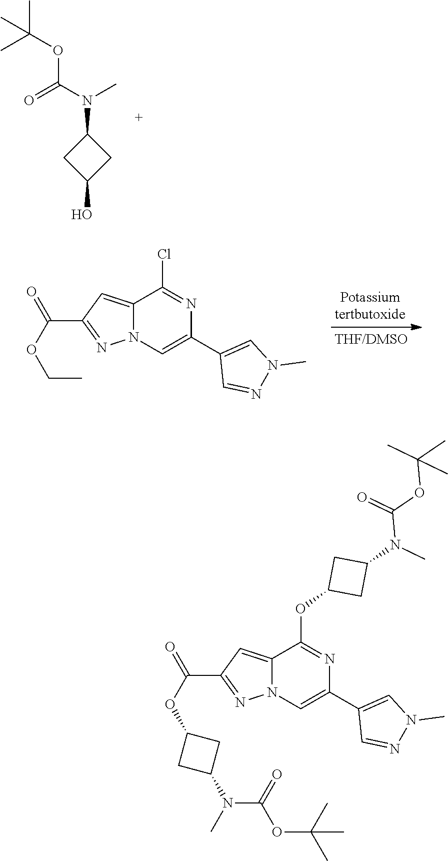 [SMILES: CN(C(=O)OC(C)(C)C)[C@H]1C[C@@H](O)C1 ; CCOC(=O)c1cc2c(Cl)nc(-c3cnn(C)c3)cn2n1 ; Cn1cc(-c2cn3nc(C(=O)O[C@H]4C[C@@H](N(C)C(=O)OC(C)(C)C)C4)cc3c(O[C@H]3C[C@@H](N(C)C(=O)OC(C)(C)C)C3)n2)cn1]

To a solution of ethyl 4-chloro-6-(1-methylpyrazol-4-yl)pyrazolo[1,5-a]pyrazine-2-carboxylate (300 mg, 981 μmol) and tert-butyl ((cis)-3-hydroxycyclobutyl)(methyl)-carbamate (494 mg, 2.45 mmol) in dry THF (4 mL) and dry DMSO (1 mL) under nitrogen was added potassium tert-butoxide solution (1 M in THF, 2.45 mL) at 0° C. The mixture was allowed to warm to RT and was stirred at RT for 2 h, quenched with H₂O (1 mL) and diluted with EtOAc. The organic layer was washed with brine (2×), dried over MgSO4, filtered, and concentrated under vacuum. The residue was purified by silica-gel chromatography (0-100% EtOAc in heptanes) to give (cis)-3-((tert-butoxycarbonyl)(methyl)amino)cyclobutyl 4-((cis)-3-((tert-butoxycarbonyl)(methyl)amino)cyclobutoxy)-6-(1-methyl-1H-pyrazol-4-yl)pyrazolo[1,5-a]pyrazine-2-carboxylate (250 mg, 41% yield) as a pale yellow foam. ¹H NMR (500 MHz, CHLOROFORM-d) δ ppm 8.23 (s, 1H), 7.74-7.92 (m, 2H), 5.55 (br s, 1H), 5.28 (br s, 1H), 3.98 (s, 3H), 3.59-3.74 (m, 2H), 3.39-3.59 (m, 5H), 3.29-3.39 (m, 1H), 1.89-2.32 (m, 10H), 1.69-1.84 (m, 2H), 1.48-1.53 (m, 18H). LCMS: m/z=471.2 [M+H]+.

### Synthesis of 4-((cis)-3-((tert-butoxycarbonyl)(methyl)amino)cyclobutoxy)-6-(1-methyl-1H-pyrazol-4-yl)pyrazolo[1,5-a]pyrazine-2-carboxylic acid

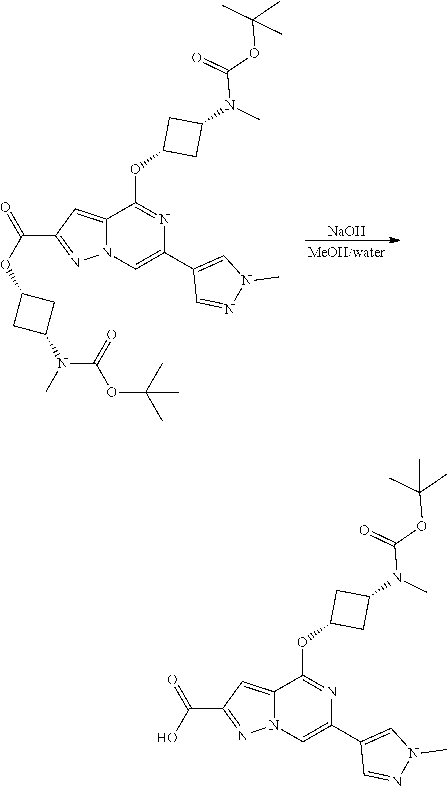 [SMILES: Cn1cc(-c2cn3nc(C(=O)O[C@H]4C[C@@H](N(C)C(=O)OC(C)(C)C)C4)cc3c(O[C@H]3C[C@@H](N(C)C(=O)OC(C)(C)C)C3)n2)cn1 ; "NaOH" ; Cn1cc(-c2cn3nc(C(=O)O)cc3c(O[C@H]3C[C@@H](N(C)C(=O)OC(C)(C)C)C3)n2)cn1]

To a solution of (cis)-3-((tert-butoxycarbonyl)(methyl)amino)cyclobutyl 4-((cis)-3-((tert-butoxycarbonyl)(methyl)amino)cyclobutoxy)-6-(1-methyl-1H-pyrazol-4-yl)pyrazolo[1,5-a]pyrazine-2-carboxylate (332 mg, 531 μmol) in methanol (1 mL) was added sodium hydroxide solution (2 M, 266 μL) and the resulting mixture was stirred at RT for 30 min. After quenching with HCl (1 M, 531 μL) and diluting with EtOAc and water, the layers were separated. The organic layer was washed with water and brine, dried (MgSO₄), filtered, and concentrated in vacuo. 4-((cis)-3-((tert-butoxycarbonyl)(methyl)amino)cyclobutoxy)-6-(1-methyl-1H-pyrazol-4-yl)pyrazolo[1,5-a]pyrazine-2-carboxylic acid was obtained as a white solid and was used directly. Quantitative yield assumed. ¹H NMR (500 MHz, DMSO-d6) δ ppm 13.26 (br s, 1H), 8.81 (s, 1H), 8.27 (s, 1H), 8.05 (s, 1H), 7.20 (s, 1H), 5.05-5.16 (m, 1H), 3.91 (s, 3H), 2.84 (m, 2H), 2.80 (s, 3H), 2.32-2.42 (m, 2H), 1.42 (s, 9H). LCMS: m/z=443.1 [M+H]+.

### Synthesis of tert-butyl ((cis)-3-((2-((tert-butoxycarbonyl)amino)-6-(1-methyl-1H-pyrazol-4-yl)pyrazolo[1,5-a]pyrazin-4-yl)oxy)cyclobutyl)(methyl)carbamate

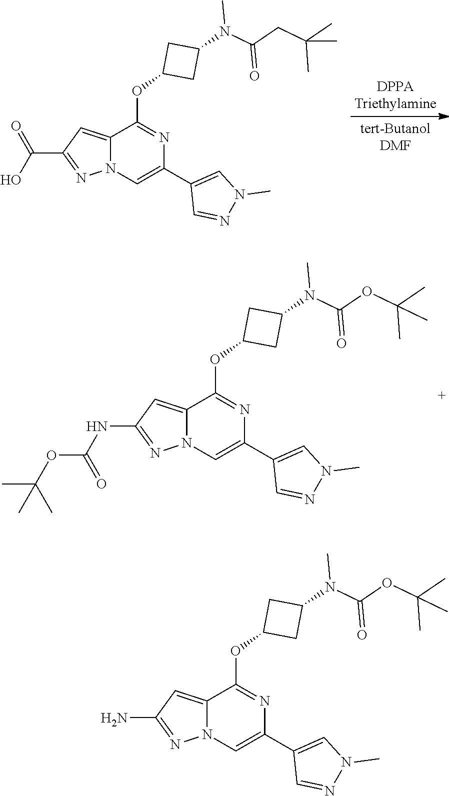

To a solution of 4-((cis)-3-((tert-butoxycarbonyl)(methyl)amino)cyclobutoxy)-6-(1-methyl-1H-pyrazol-4-yl)pyrazolo[1,5-a]pyrazine-2-carboxylic acid (115 mg, 260 μmol) in dry DMF (1 mL) and tert-butanol (0.5 mL) was added triethylamine (39 mg, 390 μmol, 54 μL), followed by DPPA (107 mg, 390 μmol, 84 μL) dropwise at rt. The resulting mixture was stirred at 80° C. for 17 h, cooled to RT, diluted with EtOAc, and washed with water and brine (3×). The organic layer was dried (MgSO₄), filtered, and concentrated in vacuo. The residue was purified by silica gel chromatography (heptanes/EtOAc 0-100%). tert-Butyl ((cis)-3-((2-((tert-butoxycarbonyl)amino)-6-(1-methyl-1H-pyrazol-4-yl)pyrazolo[1,5-a]pyrazin-4-yl)oxy)cyclobutyl)(methyl)carbamate (26 mg, 20% yield) was obtained as a white foam. ¹H NMR (500 MHz, CHLOROFORM-d) δ ppm 8.30-8.00 (br s, 1H), 8.10 (s, 1H), 7.81 (s, 1H), 7.76 (s, 1H), 6.82-7.09 (b s, 1H), 5.10 (q, J=7.2 Hz, 1H), 3.94-3.99 (m, 3H), 2.80-2.92 (m, 2H), 2.86 (s, 3H), 2.33 (m, 2H), 1.55 (s, 9H), 1.49 (s, 9H). LCMS: m/z=514.2 [M+H]+.

tert-Butyl ((cis)-3-((2-amino-6-(1-methyl-1H-pyrazol-4-yl)pyrazolo[1,5-a]pyrazin-4-yl)oxy)cyclobutyl)(methyl)carbamate (16 mg, 15% yield) was also isolated as an oil. ¹H NMR (500 MHz, CHLOROFORM-d) δ ppm 7.94 (s, 1H), 7.79 (s, 1H), 7.72 (s, 1H), 5.95 (s, 1H), 5.01-5.10 (quin, J=7.2 Hz, 1H), 4.01-4.09 (b s, 2H), 3.95 (s, 3H), 2.83-2.93 (m, 2H), 2.86 (s, 3H), 2.28-2.42 (m, 2H), 1.48 (s, 9H). LCMS: m/z=414.2 [M+H]+.

### Synthesis of 6-(1-methyl-1H-pyrazol-4-yl)-4-((cis)-3-(methylamino)cyclobutoxy)pyrazolo[1,5-a]pyrazin-2-amine

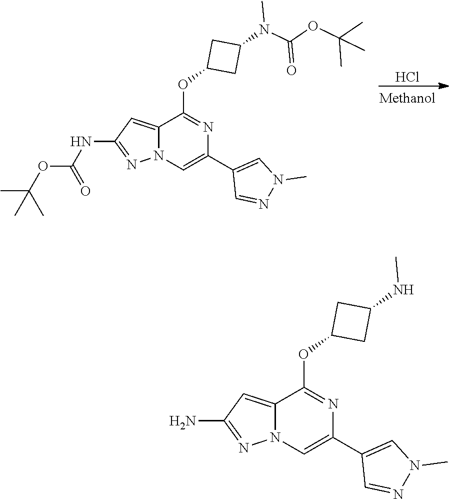 [SMILES: Cn1cc(-c2cn3nc(NC(=O)OC(C)(C)C)cc3c(O[C@H]3C[C@@H](N(C)C(=O)OC(C)(C)C)C3)n2)cn1 ; Cl ; CN[C@H]1C[C@@H](Oc2nc(-c3cnn(C)c3)cn3nc(N)cc23)C1]

A solution of tert-butyl ((cis)-3-((2-((tert-butoxycarbonyl)amino)-6-(1-methyl-1H-pyrazol-4-yl)pyrazolo[1,5-a]pyrazin-4-yl)oxy)cyclobutyl)(methyl)carbamate (26 mg, 51 μmol) in dry methanol (0.5 mL) was treated with HCl (4 M in dioxane, 506 μL). The resulting mixture was stirred at RT for 1 h. A solid formed. The mixture was concentrated in vacuo. 6-(1-methyl-1H-pyrazol-4-yl)-4-((cis)-3-(methylamino)cyclobutoxy)pyrazolo[1,5-a]pyrazin-2-amine (bishydrochloride) was obtained as a white solid, which was used directly. Quantitative yield assumed. LCMS: m/z=314.5 [M+H]+.

### Synthesis of N-((cis)-3-((2-amino-6-(1-methyl-1H-pyrazol-4-yl)pyrazolo[1,5-a]pyrazin-4-yl)oxy)cyclobutyl)-N-methylacrylamide

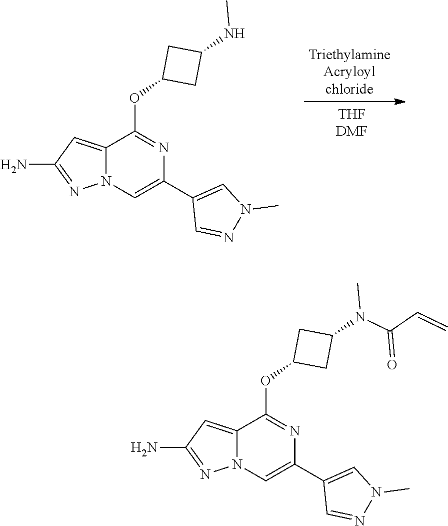 [SMILES: CN[C@H]1C[C@@H](Oc2nc(-c3cnn(C)c3)cn3nc(N)cc23)C1 ; C=CC(=O)N(C)[C@H]1C[C@@H](Oc2nc(-c3cnn(C)c3)cn3nc(N)cc23)C1]

To a suspension of crude 6-(1-methyl-1H-pyrazol-4-yl)-4-((cis)-3-(methylamino)cyclobutoxy)pyrazolo[1,5-a]pyrazin-2-amine bishydrochloride (14 mg, 36 μmol) in dry THF (0.5 mL) and dry DMF (0.5 mL) under nitrogen was added triethylamine (11 mg, 109 μmol, 15 μL) and the resulting suspension was cooled to 0° C. Acryloyl chloride (3.3 mg, 36 μmol, 3 μL) was added dropwise and the resulting mixture was stirred at 0° C. for 30 min. The reaction mixture was quenched with saturated sodium bicarbonate solution and diluted with EtOAc. The layers separated and the organic one washed with brine (3×), dried (MgSO₄), filtered, and concentrated in vacuo. The residue was purified by silica gel chromatography (0-10% MeOH in DCM). N-((cis)-3-((2-amino-6-(1-methyl-1H-pyrazol-4-yl)pyrazolo[1,5-a]pyrazin-4-yl)oxy)cyclobutyl)-N-methylacrylamide (4.4 mg, 32% yield, 95% purity) was obtained as a white foam. ¹H NMR (500 MHz, CHLOROFORM-d) δ ppm 7.94 (s, 1H), 7.78 (br s, 1H), 7.73 (br s, 1H), 6.58 (dd, J=16.8, 10.7 Hz, 1H), 6.24-6.40 (m, 1H), 5.95 (s, 1H), 5.71 (dd, J=10.4, 1.8 Hz, 1H), 5.13 (quin, J=7.2 Hz, 1H), 4.83 and 4.30 (2 br s, 1H), 4.02 (b s, 2H), 3.96 (s, 3H), 3.06 (br s, 3H), 2.90-2.98 (m, 2H), 2.51 and 2.36 (2br s, 2H). LCMS: m/z=368.1 [M+H]+.

### Example 69: (S)-1-(4-((6-(1,3-dimethyl-1H-pyrazol-4-yl)pyrazolo[1,5-a]pyrazin-4-yl)oxy)azepan-1-yl)prop-2-en-1-one and (R)-1-(4-((6-(1,3-dimethyl-1H-pyrazol-4-yl)pyrazolo[1,5-a]pyrazin-4-yl)oxy)azepan-1-yl)prop-2-en-1-one

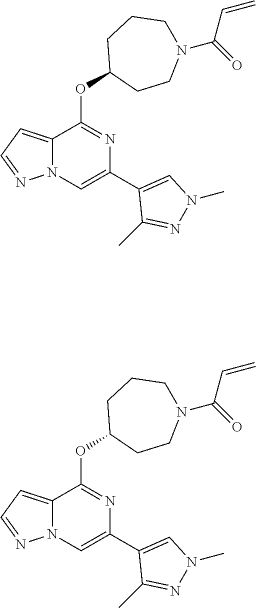 [SMILES: C=CC(=O)N1CCC[C@H](Oc2nc(-c3cn(C)nc3C)cn3nccc23)CC1 ; C=CC(=O)N1CCC[C@@H](Oc2nc(-c3cn(C)nc3C)cn3nccc23)CC1]

### Synthesis of tert-butyl-4-(6-chloropyrazolo[1,5-a]pyrazin-4-yl)oxyazepane-1-carboxylate

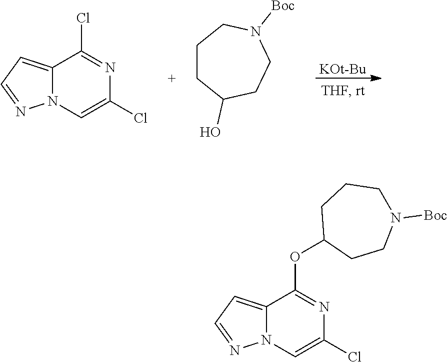 [SMILES: Clc1cn2nccc2c(Cl)n1 ; CC(C)(C)OC(=O)N1CCCC(O)CC1 ; CC(C)(C)OC(=O)N1CCCC(Oc2nc(Cl)cn3nccc23)CC1 ; "KOt-Bu"]

To a solution of tert-butyl-4-hydroxyazepane-1-carboxylate (4.0 g, 18.6 mmol) and 4,6-dichloropyrazolo[1,5-a]pyrazine (3.5 g, 18.6 mmol) in THF (100 mL) was slowly added a solution of potassium tert-butoxide (1 M in THF, 18.6 mL, 18.6 mmol). The flask was stirred at room temperature for 2 hours. The material was concentrated to half volume and was taken up in EtOAc and water. The organic layer was washed with brine, dried over anhydrous sodium sulfate, filtered, and concentrated. The residue was purified via 80 g silica gel column using a gradient of 0-60% EtOAc in heptanes. Relevant fractions were combined to afford tert-butyl-4-(6-chloropyrazolo[1,5-a]pyrazin-4-yl)oxyazepane-1-carboxylate (4.32 g, 63% yield) as a yellow oil. LCMS m/z=367.1 (M+H)⁺. ¹H NMR (400 MHz, DMSO-d₆) δ ppm 1.36-1.47 (m, 9H) 1.68 (br dd, J=11.7, 5.4 Hz, 1H) 1.83 (br s, 1H) 1.90-1.98 (m, 3H) 2.03-2.24 (m, 1H) 3.34-3.53 (m, 4H) 5.22-5.44 (m, 1H) 6.88-6.98 (m, 1H) 8.01-8.14 (m, 1H) 8.65-8.76 (m, 1H)

### Synthesis of tert-butyl-4-[6-(1,3-dimethylpyrazol-4-yl)pyrazolo[1,5-a]pyrazin-4-yl]oxyazepane-1-carboxylate

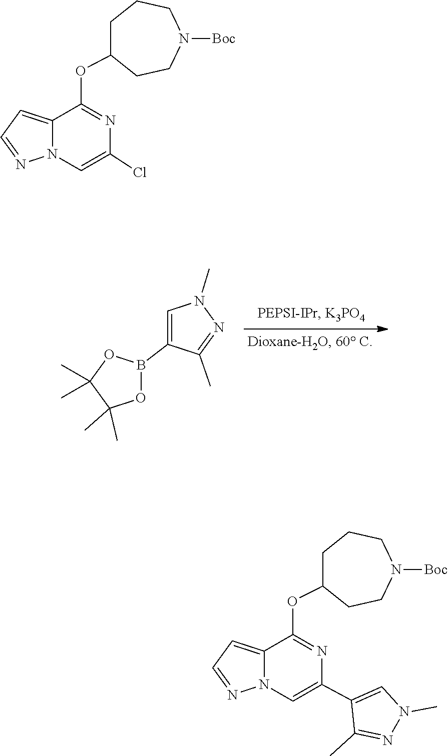 [SMILES: CC(C)(C)OC(=O)N1CCCC(Oc2nc(Cl)cn3nccc23)CC1 ; Cc1nn(C)cc1B1OC(C)(C)C(C)(C)O1 ; Cc1nn(C)cc1-c1cn2nccc2c(OC2CCCN(C(=O)OC(C)(C)C)CC2)n1]

To a microwave vial was added tert-butyl-4-(6-chloropyrazolo[1,5-a]pyrazin-4-yl)oxyazepane-1-carboxylate (1 M, 0.55 mL, 0.55 mmol), 1,3-dimethyl-4-(4,4,5,5-tetramethyl-1,3,2-dioxaborolan-2-yl)pyrazole (145 mg, 0.65 mmol), K₃PO₄ (1 M in water, 1.09 mL), Pd-PEPPSI™-IPr (37 mg, 55 μmol) and dioxane (5 mL). The vial was capped and stirred at 60° C. overnight. The reaction mixture was concentrated and purified via 12 g silica gel column using a gradient of 40-70% EtOAc in heptanes. Relevant fractions were combined afford tert-butyl-4-[6-(1,3-dimethylpyrazol-4-yl)pyrazolo[1,5-a]pyrazin-4-yl]oxyazepane-1-carboxylate (136 mg, 58% yield) as a pale yellow oil. LCMS m/z=427.2 (M+H)+.

### Synthesis of 4-[azepan-4-yl]oxy-6-(1,3-dimethylpyrazol-4-yl)pyrazolo[1,5-a]pyrazine hydrochloride

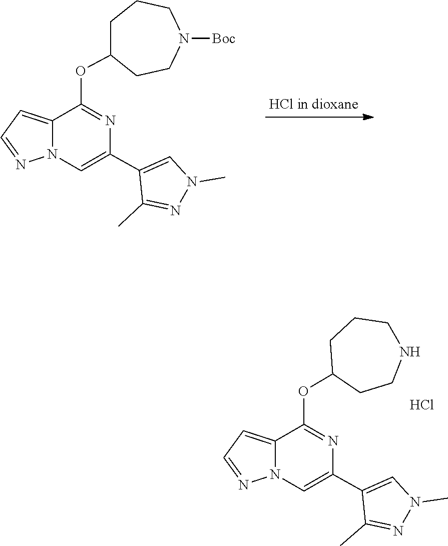 [SMILES: Cc1nn(C)cc1-c1cn2nccc2c(OC2CCCN(C(=O)OC(C)(C)C)CC2)n1 ; Cc1nn(C)cc1-c1cn2nccc2c(OC2CCCNCC2)n1 ; Cl]

To a solution of tert-butyl-4-[6-(1,3-dimethylpyrazol-4-yl)pyrazolo[1,5-a]pyrazin-4-yl]oxyazepane-1-carboxylate (136 mg, 0.32 mmol) in dioxane (2 mL) was added HCl (4 M in dioxane, 0.8 mL). The mixture was stirred at room temperature overnight. The material was concentrated to afford 4-[azepan-4-yl]oxy-6-(1,3-dimethylpyrazol-4-yl)pyrazolo[1,5-a]pyrazine hydrochloride (115 mg, crude) as an off white solid. LCMS m/z=327.1 (M+H)+.

### Synthesis of (S)-1-(4-((6-(1,3-dimethyl-1H-pyrazol-4-yl)pyrazolo[1,5-a]pyrazin-4-yl)oxy)azepan-1-yl)prop-2-en-1-one and (R)-1-(4-((6-(1,3-dimethyl-1H-pyrazol-4-yl)pyrazolo[1,5-a]pyrazin-4-yl)oxy)azepan-1-yl)prop-2-en-1-one

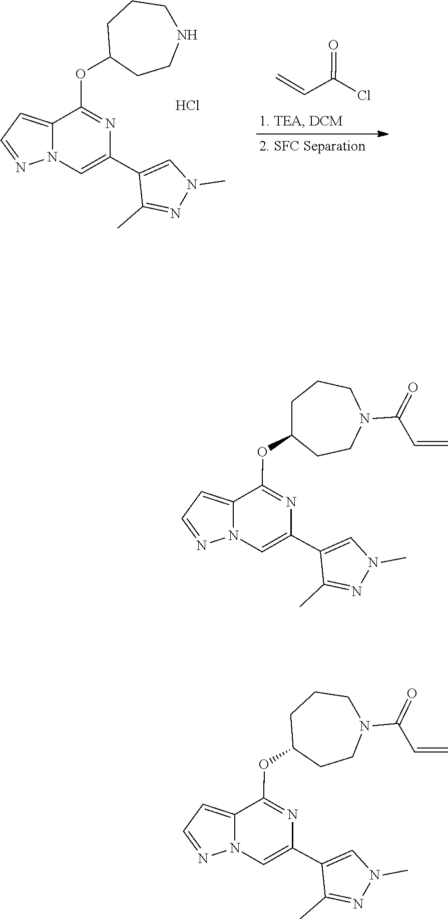

To a vial was added 4-[azepan-4-yl]oxy-6-(1,3-dimethylpyrazol-4-yl)pyrazolo[1,5-a]pyrazine hydrochloride (115 mg, 0.32 mmol) in DCM (4 mL), triethylamine (193 mg, 1.9 mmol, 266 μL) and prop-2-enoyl chloride (35 mg, 0.38 mmol, 31 μL) in that order. The vial was stirred at room temperature overnight. The reaction mixture was concentrated, taken up in DMSO and passed through a plug. The material was purified via reverse phase purification (Column: Waters XSelect CSH Prep C18 5 um OBD 19×100 mm; Condition: 5-50% Acetonitrile in 0.1% v/v Ammonium carbonate/water; Flowrate: 30 mL/min) to afford 60.6 mg (50% yield) of desired product. LCMS m/z=381.2 (M+H)+. ¹H NMR (500 MHz, DMSO-d₆) δ ppm 1.67-1.80 (m, 1H) 1.86-2.10 (m, 4H) 2.16-2.26 (m, 1H) 2.42 (s, 3H) 3.56-3.78 (m, 4H) 3.81 (d, J=1.22 Hz, 3H) 5.42-5.56 (m, 1H) 5.65-5.74 (m, 1H) 6.13-6.21 (m, 1H) 6.75-6.87 (m, 2H) 7.98-8.04 (m, 1H) 8.08-8.15 (m, 1H) 8.42-8.49 (m, 1H).

The material was chirally purified using following conditions (column: CHIRALPAK AD-H 30×250 mm, 5 um; Method: 30% MeOH w/no modifier in CO₂; Flowrate: 100 mL/min; ABPR: 120 bar; MBPR: 40 PSI; Column Temp: 40° C.). The first eluting peak E1 was concentrated to afford 11.3 mg of one enantiomer of 1-[-4-[6-(1,3-dimethylpyrazol-4-yl)pyrazolo[1,5-a]pyrazin-4-yl]oxyazepan-1-yl]prop-2-en-1-one as a white solid. LCMS m/z=381.2 (M+H)⁺. The second eluting peak E2 was concentrated to afford 5.4 mg of the second enantiomer of 1-[4-[6-(1,3-dimethylpyrazol-4-yl)pyrazolo[1,5-a]pyrazin-4-yl]oxyazepan-1-yl]prop-2-en-1-one as a white solid. LCMS m/z=381.2 (M+H)⁺. The stereochemistry of the two isomers was not assigned.

### Example 70: 1-[(4R)-4-[[6-(1,3-dimethylpyrazol-4-yl)-[1,2,4]triazolo[1,5-a]pyrazin-8-yl]oxy]azepan-1-yl]prop-2-en-1-one

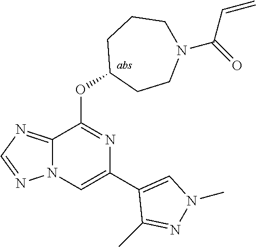 [SMILES: C=CC(=O)N1CCC[C@@H](Oc2nc(-c3cn(C)nc3C)cn3ncnc23)CC1]

### Synthesis of tert-butyl (4R) 4-((6-bromo-[1,2,4]triazolo[1,5-a]pyrazin-8-yl)oxy)azepane-1-carboxylate

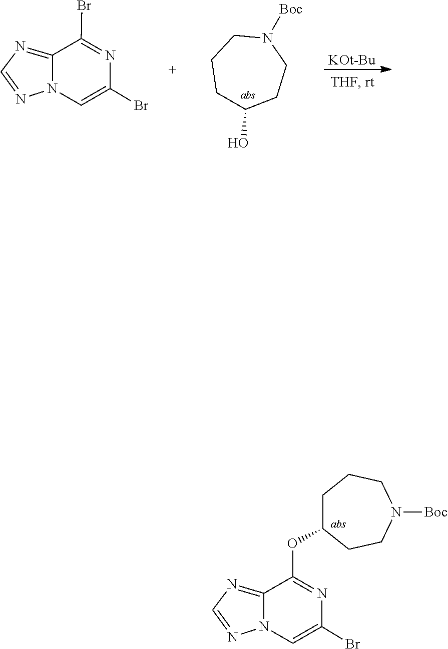

To a solution of tert-butyl (4R)-4-hydroxyazepane-1-carboxylate (775 mg, 3.60 mmol) and 6,8-dibromo-[1,2,4]triazolo[1,5-a]pyrazine (1 g, 3.60 mmol) in THF (36 mL) was slowly added a solution of potassium tert-butoxide (1 M in THF, 3.6 mL, 3.6 mmol). The flask was stirred at room temperature for 1 hour. The material was concentrated to half volume and was taken up in EtOAc and water. The organic layer was washed with brine dried over anhydrous sodium sulfate, filtered, and concentrated. The residue was purified via 40 g silica gel column using a gradient of 10-70% EtOAc in heptanes. Relevant fractions were combined to afford tert-butyl (4R)-4-[(6-bromo-[1,2,4]triazolo[1,5-a]pyrazin-8-yl)oxy]azepane-1-carboxylate (1.48 g, 2.79 mmol, 77.5% yield) as a white foam. LCMS m/z=414.0 (M+H)+. ¹H NMR (500 MHz, DMSO-d₆) δ ppm 1.43 (s, 9H) 1.69 (br dd, J=8.85, 4.58 Hz, 1H) 1.81-2.04 (m, 4H) 2.10-2.31 (m, 1H) 3.37-3.53 (m, 4H) 5.27-5.39 (m, 1H) 8.52-8.66 (m, 1H) 8.97-9.15 (m, 1H)

### Synthesis of tert-butyl (4R)-4-[[6-(1,3-dimethylpyrazol-4-yl)-[1,2,4]triazolo[1,5-a]pyrazin-8-yl]oxy]azepane-1-carboxylate

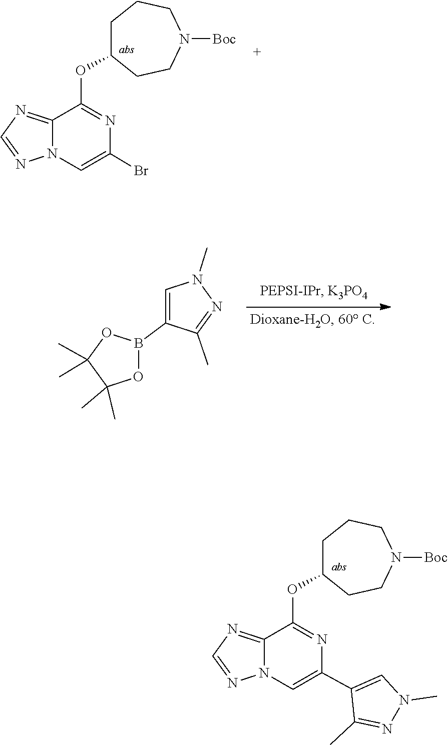

To a microwave vial was added tert-butyl (4R)-4-[(6-bromo-[1,2,4]triazolo[1,5-a]pyrazin-8-yl)oxy]azepane-1-carboxylate (230 mg, 0.56 mmol), 1,3-dimethyl-4-(4,4,5,5-tetramethyl-1,3,2-dioxaborolan-2-yl)pyrazole (149 mg, 0.67 mmol), K₃PO₄ (1 M in water, 1.12 mL), Pd-PEPPSI™-IPr (38 mg, 55.8 μmol) and dioxane (5.00 mL). The vial was capped and stirred at 70° C. overnight. The reaction mixture was concentrated and purified via 12 g silica gel column using a gradient of 30-100% EtOAc in heptanes. Relevant fractions were combined afford tert-butyl (4R)-4-[[6-(1,3-dimethylpyrazol-4-yl)-[1,2,4]triazolo[1,5-a]pyrazin-8-yl]oxy]azepane-1-carboxylate (65 mg, 27% yield) as a pale yellow oil. LCMS m/z=428.2 (M+H)+

### Synthesis of 8-[(4R)-azepan-4-yl]oxy-6-(1,3-dimethylpyrazol-4-yl)-[1,2,4]triazolo[1,5-a]pyrazine hydrochloride

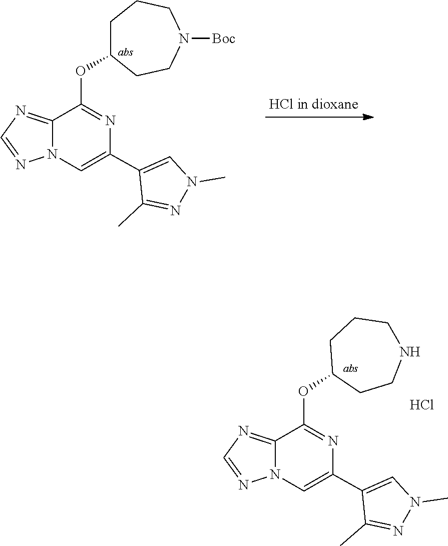 [SMILES: Cc1nn(C)cc1-c1cn2ncnc2c(O[C@@H]2CCCN(C(=O)OC(C)(C)C)CC2)n1 ; Cc1nn(C)cc1-c1cn2ncnc2c(O[C@@H]2CCCNCC2)n1 ; Cl]

To a solution of tert-butyl (4R)-4-[[6-(1,3-dimethylpyrazol-4-yl)-[1,2,4]triazolo[1,5-a]pyrazin-8-yl]oxy]azepane-1-carboxylate (65 mg, 0.15 mmol) in dioxane (5 mL) was added HCl (4 M in dioxane, 0.38 mL). The mixture was stirred at room temperature overnight. The material was concentrated to afford 8-[(4R)-azepan-4-yl]oxy-6-(1,3-dimethylpyrazol-4-yl)-[1,2,4]triazolo[1,5-a]pyrazine hydrochloride (55.3 mg, 100% yield) as an off white solid. LCMS m/z=328.1 (M+H)+.

### Synthesis of 1-[(4R)-4-[[6-(1,3-dimethylpyrazol-4-yl)-[1,2,4]triazolo[1,5-a]pyrazin-8-yl]oxy]azepan-1-yl]prop-2-en-1-one

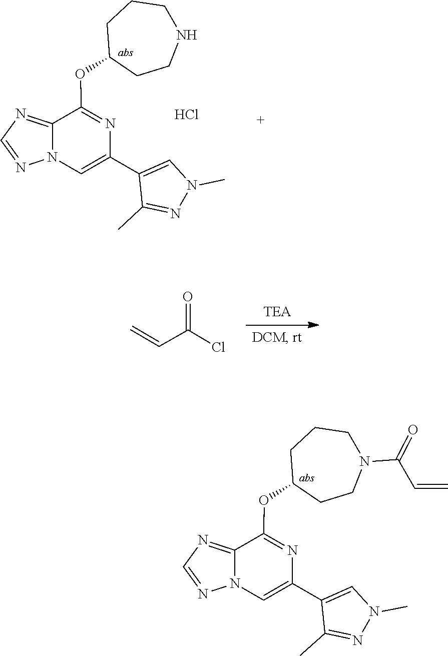

To a vial was added 8-[(4R)-azepan-4-yl]oxy-6-(1,3-dimethylpyrazol-4-yl)-[1,2,4]triazolo[1,5-a]pyrazine hydrochloride (55.3 mg, 0.15 mmol) in DCM (4 mL), triethylamine (93 mg, 0.92 mmol, 128 μL) and prop-2-enoyl chloride (17 mg, 0.18 mmol, 15 μL) in that order. The vial was stirred at room temperature overnight. The reaction mixture was concentrated, taken up in DMSO and passed through a plug. The material was purified via reverse phase purification (Column: Waters XSelect CSH Prep C18 5 um OBD 19×100 mm; Condition: 5-40% Acetonitrile in 0.1% v/v Ammonium carbonate/water; Flowrate: 30 mL/min) to afford 26.4 mg (45% yield) of desired product. LCMS m/z=382.3 (M+H)⁺. ¹H NMR (500 MHz, DMSO-d₆) δ ppm 1.69-1.81 (m, 1H) 1.91-2.07 (m, 4H) 2.21-2.31 (m, 1H) 2.44 (d, J=1.22 Hz, 3H) 3.49-3.59 (m, 2H) 3.66-3.76 (m, 2H) 3.82 (d, J=1.83 Hz, 3H) 5.41-5.54 (m, 1H) 5.65-5.77 (m, 1H) 6.11-6.22 (m, 1H) 6.75-6.88 (m, 1H) 8.11-8.23 (m, 1H) 8.52-8.61 (m, 1H) 8.69-8.78 (m, 1H).

### Example 71: (S)-5-(4-((1-acryloylazepan-4-yl)oxy)pyrazolo[1,5-a]pyrazin-6-yl)-1-methylpyridin-2(1H)-one and (R)-5-(4-((1-acryloylazepan-4-yl)oxy)pyrazolo[1,5-a]pyrazin-6-yl)-1-methylpyridin-2(1H)-one

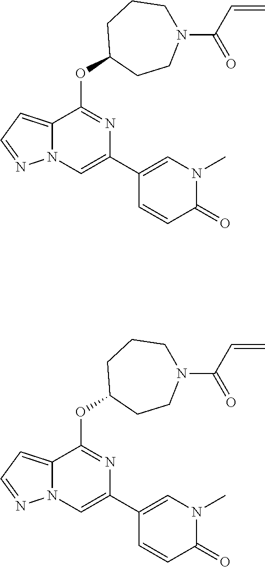 [SMILES: C=CC(=O)N1CCC[C@H](Oc2nc(-c3ccc(=O)n(C)c3)cn3nccc23)CC1 ; C=CC(=O)N1CCC[C@@H](Oc2nc(-c3ccc(=O)n(C)c3)cn3nccc23)CC1]

### Synthesis of tert-butyl 4-((6-(1-methyl-6-oxo-1,6-dihydropyridin-3-yl)pyrazolo[1,5-a]pyrazin-4-yl)oxy)azepane-1-carboxylate

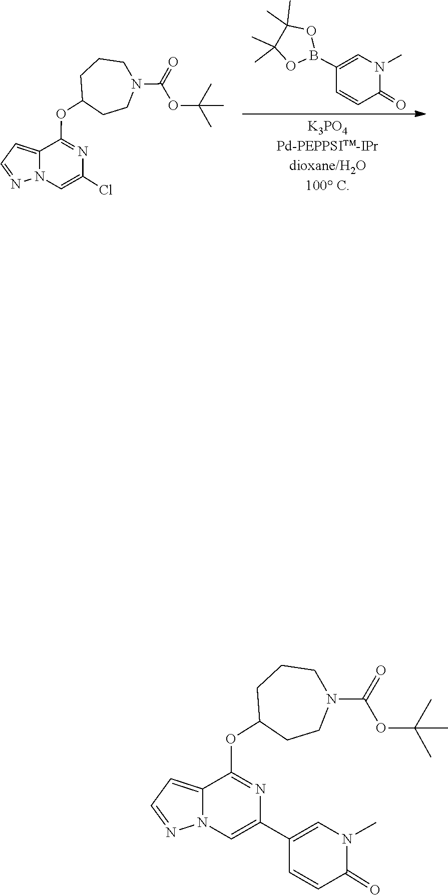

To a 20-mL scintillation vial containing tert-butyl 4-((6-chloropyrazolo[1,5-a]pyrazin-4-yl)oxy)azepane-1-carboxylate (200 mg, 0.55 mmol) in dioxane (5.0 mL) was added 1-methyl-5-(4,4,5,5-tetramethyl-1,3,2-dioxaborolan-2-yl)pyridin-2(1H)-one (154 mg, 0.64 mmol). Next, aq. K₃PO₄ (1 M, 1.09 mmol, 1.1 mL) was added to the reaction mixture, followed by Pd-PEPPSI™-IPr (37 mg, 55 μmol). The vial was purged with N₂ and heated at 100° C. overnight. After this time, the reaction mixture was filtered through a pad of Celite® and concentrated under reduced pressure to afford an amber oil. The crude material was purified by silica gel chromatography (0 to 25% EtOAc in heptanes then 100% [3:1 EtOAc EtOH]) to afford tert-butyl 4-((6-(1-methyl-6-oxo-1,6-dihydropyridin-3-yl)pyrazolo[1,5-a]pyrazin-4-yl)oxy)azepane-1-carboxylate (155 mg, 65% yield) as an off-white solid. LC-MS: m/z=440.0 (M+H)⁺.

### Synthesis of 5-(4-(azepan-4-yloxy)pyrazolo[1,5-a]pyrazin-6-yl)-1-methylpyridin-2(1H)-one

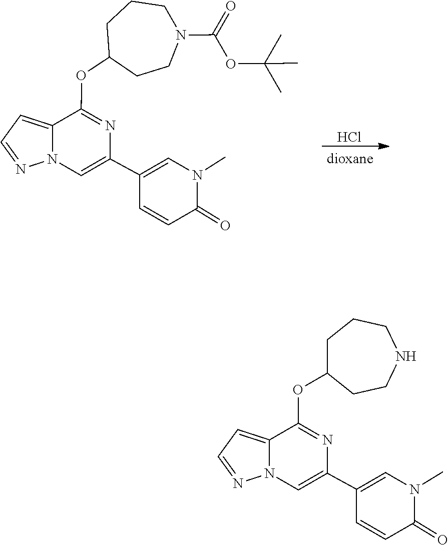 [SMILES: Cn1cc(-c2cn3nccc3c(OC3CCCN(C(=O)OC(C)(C)C)CC3)n2)ccc1=O ; Cl ; Cn1cc(-c2cn3nccc3c(OC3CCCNCC3)n2)ccc1=O]

To a solution of tert-butyl 4-((6-(1-methyl-6-oxo-1,6-dihydropyridin-3-yl)pyrazolo[1,5-a]pyrazin-4-yl)oxy)azepane-1-carboxylate (155 mg, 0.35 mmol) in dioxane (1.4 mL) was added HCl (4 M in dioxane, 883 μL, 3.5 mmol). The reaction mixture immediately became heterogeneous upon addition of the HCl solution and was stirred at RT for 1.5 h. The reaction mixture was directly concentrated under reduced pressure to afford the title compound as an orange solid which was taken forward without further purification assuming 100% yield. LC-MS: m/z=361.9 (M+Na)⁺.

### Synthesis of (S)-5-(4-((1-acryloylazepan-4-yl)oxy)pyrazolo[1,5-a]pyrazin-6-yl)-1-methylpyridin-2(1H)-one and (R)-5-(4-((1-acryloylazepan-4-yl)oxy)pyrazolo[1,5-a]pyrazin-6-yl)-1-methylpyridin-2(1H)-one

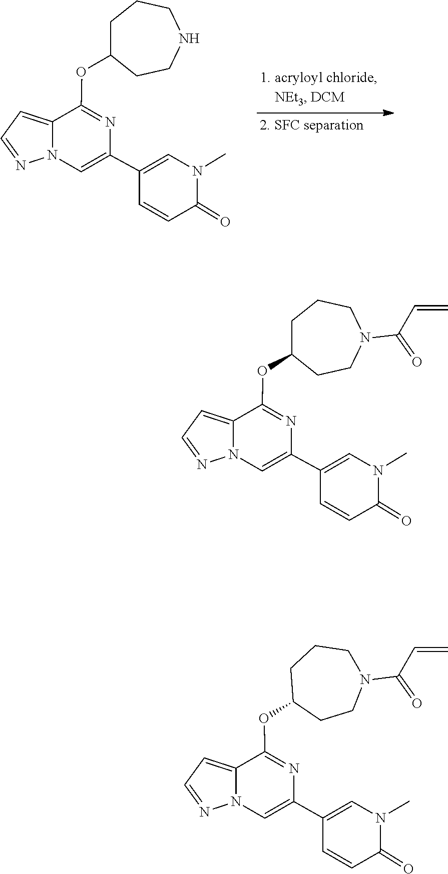 [SMILES: Cn1cc(-c2cn3nccc3c(OC3CCCNCC3)n2)ccc1=O ; C=CC(=O)N1CCC[C@H](Oc2nc(-c3ccc(=O)n(C)c3)cn3nccc23)CC1 ; C=CC(=O)N1CCC[C@@H](Oc2nc(-c3ccc(=O)n(C)c3)cn3nccc23)CC1]

To a solution of 5-(4-(azepan-4-yloxy)pyrazolo[1,5-a]pyrazin-6-yl)-1-methylpyridin-2(1H)-one (120 mg, 0.35 mmol) in DCM (1.4 mL) was added triethylamine (99 μL, 0.71 mmol) followed by acryloyl chloride (57 μL, 0.71 mmol). The solution became red and homogeneous upon addition of acryloyl chloride and was stirred at RT for 20 mins. The reaction mixture was then concentrated in vacuo and loaded onto a silica gel cartridge. The crude material was purified by silica gel chromatography (0 to 100% EtOAc in heptanes then 0 to 15% MeOH in heptanes) to afford 5-(4-((1-acryloylazepan-4-yl)oxy)pyrazolo[1,5-a]pyrazin-6-yl)-1-methylpyridin-2(1H)-one as a white solid (52.1 mg, 38% over 2 steps). LC-MS: m/z=393.9 (M+H)⁺. ¹H NMR (400 MHz, CDCl₃) δ ppm 1.72 (br s, 1H) 1.74-1.91 (m, 2H) 1.95-2.49 (m, 6H) 3.46-3.64 (m, 2H) 3.65-3.73 (m, 4H) 3.77-4.12 (m, 2H) 5.46-5.65 (m, 1H) 5.69-5.75 (m, 1H) 6.35-6.43 (m, 1H) 6.58-6.70 (m, 2H) 6.73-6.78 (m, 1H) 7.74-7.79 (m, 1H) 7.91 (dd, J=4.39, 2.38 Hz, 1H) 7.95-8.11 (m, 1H) 8.23 (d, J=0.75 Hz, 1H).

The racemic material was separated by chiral SFC (Chiralpak AD-H 30×250 mm, 5 μm column; 25% MeOH in CO₂ with no modifier; flow rate=100 mL/min, ABPR 120 bar, MBPR 40 psi, column temp. 40° C.), providing enantiomer the first eluting enantiomer E1 (7.0 mg, 100% ee, Rf=4.36 min) and the second eluting enantiomer E2 (arbitrarily assigned as R 7.6 mg, 90% ee, Rf=4.77 min).

### Example 72: 1-(4-((6-(2-methoxypyridin-4-yl)pyrazolo[1,5-a]pyrazin-4-yl)oxy)azepan-1-yl)prop-2-en-1-one (chiral, but absolute chemistry not known)

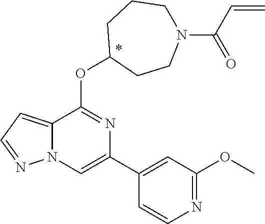 [SMILES: C=CC(=O)N1CCCC(Oc2nc(-c3ccnc(OC)c3)cn3nccc23)CC1]

### Synthesis of tert-butyl 4-((6-(2-methoxypyridin-4-yl)pyrazolo[1,5-a]pyrazin-4-yl)oxy)azepane-1-carboxylate

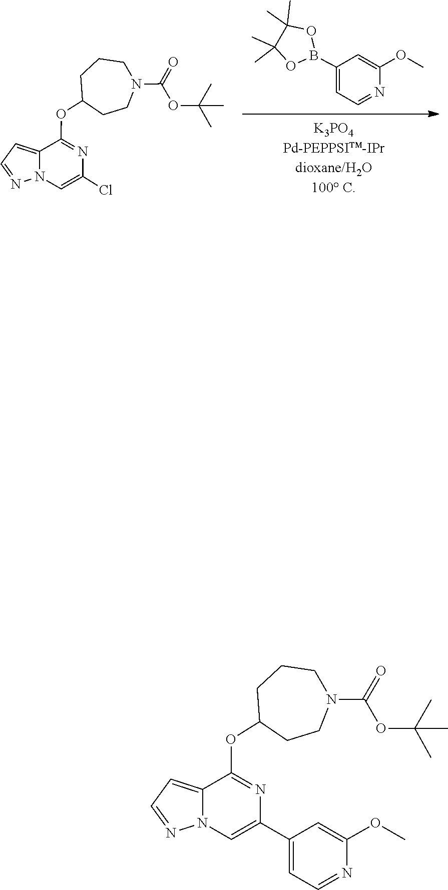

To a 20-mL scintillation vial containing tert-butyl 4-((6-chloropyrazolo[1,5-a]pyrazin-4-yl)oxy)azepane-1-carboxylate (200 mg, 0.55 mmol) in dioxane (5.0 mL) was added 2-methoxy-4-(4,4,5,5-tetramethyl-1,3,2-dioxaborolan-2-yl)pyridine (100 mg, 0.65 mmol). A solution of aq. K₃PO₄ (1 M, 1.09 mmol, 1.09 mL) was then added to the reaction mixture followed by Pd-PEPPSI™_IPr (37 mg, 55 μmol). The vial was purged with N₂ and heated at 100° C. overnight. The reaction mixture was returned to RT and then filtered through a pad of Celite®. Concentration under reduced pressure provided the crude product as an amber oil. The crude material was purified by silica gel chromatography (0 to 25% EtOAc in heptanes) to afford tert-butyl 4-((6-(2-methoxypyridin-4-yl)pyrazolo[1,5-a]pyrazin-4-yl)oxy)azepane-1-carboxylate (184 mg, 77% yield) as an off-white solid. LC-MS: m/z=440.0 (M+H)⁺.

### Synthesis of 4-(azepan-4-yloxy)-6-(2-methoxypyridin-4-yl)pyrazolo[1,5-a]pyrazine

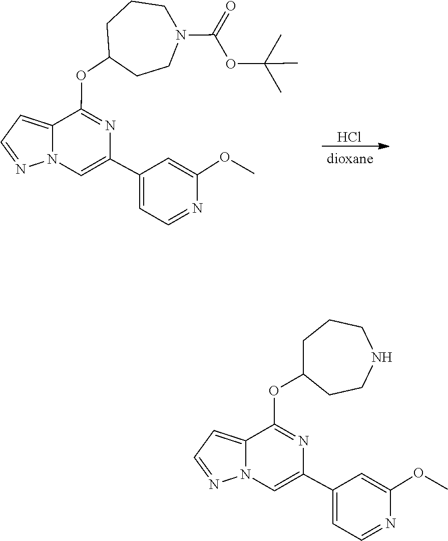 [SMILES: COc1cc(-c2cn3nccc3c(OC3CCCN(C(=O)OC(C)(C)C)CC3)n2)ccn1 ; Cl ; COc1cc(-c2cn3nccc3c(OC3CCCNCC3)n2)ccn1]

To a solution of tert-butyl 4-((6-(2-methoxypyridin-4-yl)pyrazolo[1,5-a]pyrazin-4-yl)oxy)azepane-1-carboxylate (184 mg, 0.42 mmol) in dioxane (1.7 mL) was added a solution of HCl (4 M in dioxane, 1.05 mL, 4.2 mmol). The reaction mixture was stirred at RT for 3 h and then concentrated in vacuo to provide crude 4-(azepan-4-yloxy)-6-(2-methoxypyridin-4-yl)pyrazolo[1,5-a]pyrazine as a bright yellow solid. The crude product was used without further purification assuming 100% yield. LC-MS: m/z=340.0 (M+H)⁺.

### Synthesis of chiral 1-(4-((6-(2-methoxypyridin-4-yl)pyrazolo[1,5-a]pyrazin-4-yl)oxy)azepan-1-yl)prop-2-en-1-one

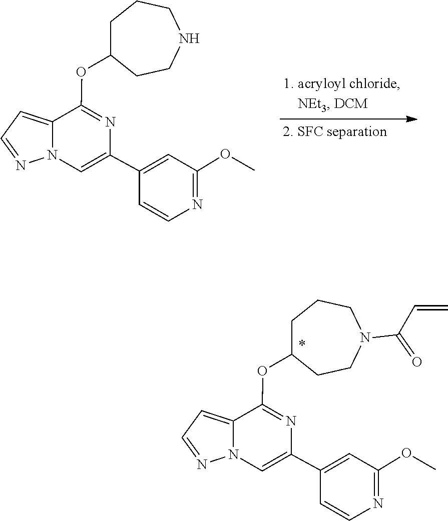 [SMILES: COc1cc(-c2cn3nccc3c(OC3CCCNCC3)n2)ccn1 ; C=CC(=O)N1CCCC(Oc2nc(-c3ccnc(OC)c3)cn3nccc23)CC1]

To a solution of crude 4-(azepan-4-yloxy)-6-(2-methoxypyridin-4-yl)pyrazolo[1,5-a]pyrazine (142 mg, 0.42 mmol) in DCM (1.7 mL) was added triethylamine (0.29 mL, 2.09 mmol) at RT followed immediately by acryloyl chloride (68 μL, 0.84 mmol). The reaction mixture became deep red and homogeneous and was stirred at RT for 20 mins. After this time, the reaction mixture was quenched by addition of sat. aq. NaHCO₃ and diluted with EtOAc. The resulting layers were separated, and the aqueous layer was further extracted (3×) with EtOAc. The combined organics were dried over anhydrous sodium sulfate and concentrated under reduced pressure. The crude material was purified by silica gel chromatography (0 to 100% EtOAc in heptanes) to afford 1-(4-((6-(2-methoxypyridin-4-yl)pyrazolo[1,5-a]pyrazin-4-yl)oxy)azepan-1-yl)prop-2-en-1-one (64.5 mg, 39% over 2 steps) as a colorless oil. LC-MS: m/z=393.9 (M+H)⁺. ¹H NMR (400 MHz, CDCl₃) δ ppm 1.81-1.92 (m, 1H) 1.97-2.13 (m, 2H) 2.16-2.31 (m, 3H) 3.62-3.85 (m, 4H) 4.01-4.04 (m, 3H) 5.64-5.70 (m, 1H) 5.72 (dd, J=10.29, 2.01 Hz, 1H) 6.39 (ddd, J=16.75, 7.72, 2.13 Hz, 1H) 6.57-6.68 (m, 1H) 6.80 (dd, J=4.02, 2.01 Hz, 1H) 7.31-7.39 (m, 2H) 7.98 (t, J=2.01 Hz, 1H) 8.24 (d, J=5.27 Hz, 1H) 8.56 (s, 1H).

The racemic material was separated by chiral SFC (Chiralpak IB 30×250 mm, 5 μm column; 15% MeOH in CO₂ with no modifier; flow rate=100 mL/min, ABPR 120 bar, MBPR 40 psi, column temp. 40° C.), providing a first eluting enantiomer E1 (12.2 mg, 100% ee, Rf=6.91 min) and the second eluting enantiomer E2 (3.6 mg, 96% ee, Rf=7.45 min). The second enantiomer contained an inseparable impurity and was not further purified.

### Example 73: (R)-1-(4-((6-phenylpyrazolo[1,5-a]pyrazin-4-yl)oxy)azepan-1-yl)prop-2-en-1-one

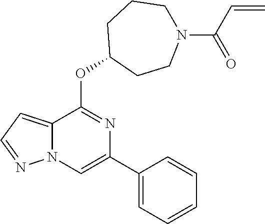 [SMILES: C=CC(=O)N1CCC[C@@H](Oc2nc(-c3ccccc3)cn3nccc23)CC1]

### Synthesis of tert-butyl (R)-4-((6-(4,4,5,5-tetramethyl-1,3,2-dioxaborolan-2-yl)pyrazolo[1,5-a]pyrazin-4-yl)oxy)azepane-1-carboxylate

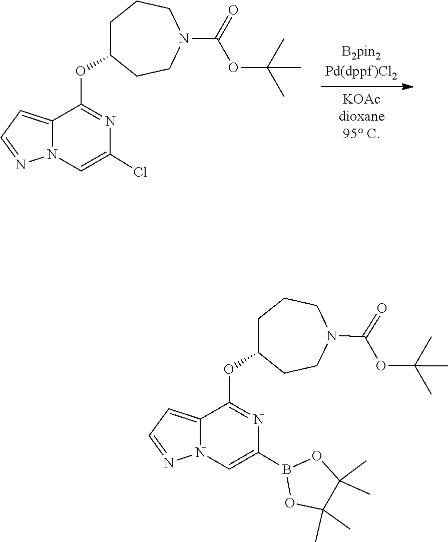 [SMILES: CC(C)(C)OC(=O)N1CCC[C@@H](Oc2nc(Cl)cn3nccc23)CC1 ; CC(C)(C)OC(=O)N1CCC[C@@H](Oc2nc(B3OC(C)(C)C(C)(C)O3)cn3nccc23)CC1]

To a 20-mL scintillation vial containing tert-butyl (R)-4-((6-chloropyrazolo[1,5-a]pyrazin-4-yl)oxy)azepane-1-carboxylate (734 mg, 2.0 mmol) dissolved in dioxane (10 mL) was added bis(pinacolato)diboron (610 mg, 2.4 mmol). Next, KOAc (589 mg, 6.0 mmol) was added to the reaction mixture, followed by [1,1′-bis(diphenylphosphino)ferrocene]dichloropalladium(II) (293 mg, 0.40 mmol). The vial was purged with N₂ and then stirred overnight at 95° C. After this time, the reaction mixture was cooled to RT and filtered through a pad of Celite® with the aid of EtOAc. The crude material was dry-loaded onto silica gel and purified by silica gel chromatography (0 to 40% EtOAc in heptanes) to provide tert-butyl (R)-4-((6-(4,4,5,5-tetramethyl-1,3,2-dioxaborolan-2-yl)pyrazolo[1,5-a]pyrazin-4-yl)oxy)azepane-1-carboxylate (744 mg, 81% yield) as a colorless oil. LC-MS: m/z=399.2 (M-86+Na)⁺.

### Synthesis of tert-butyl (R)-4-((6-phenylpyrazolo[1,5-a]pyrazin-4-yl)oxy)azepane-1-carboxylate

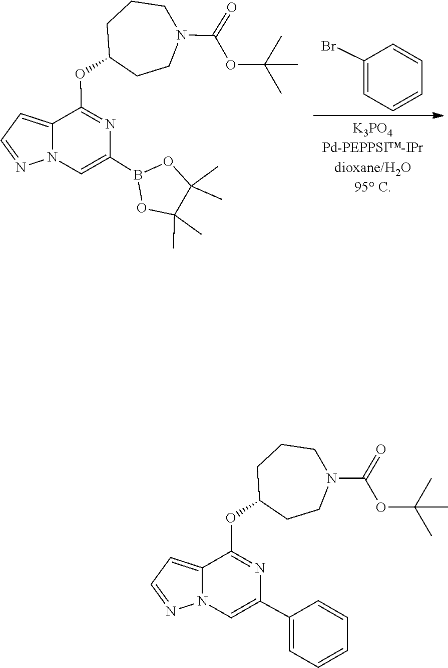

To a 20-mL scintillation vial containing tert-butyl (R)-4-((6-(4,4,5,5-tetramethyl-1,3,2-dioxaborolan-2-yl)pyrazolo[1,5-a]pyrazin-4-yl)oxy)azepane-1-carboxylate (248 mg, 0.54 mmol) in dioxane (1.8 mL) was added bromobenzene (38 μL, 0.36 mmol) followed sequentially by a solution of aq. K₃PO₄ (0.5 M, 1.44 mL, 0.72 mmol) and Pd-PEPPSI™-IPr (49 mg, 72 μmol). The reaction mixture was heated at 95° C. overnight, after which time it was cooled to RT and directly concentrated under reduced pressure. The crude material was purified by silica gel chromatography (0 to 100% EtOAc in heptanes) to provide tert-butyl (R)-4-((6-phenylpyrazolo[1,5-a]pyrazin-4-yl)oxy)azepane-1-carboxylate (47.6 mg, 32% yield) as a yellow oil. LC-MS: m/z=409.2 (M+H)⁺.

### Synthesis of (R)-4-(azepan-4-yloxy)-6-phenylpyrazolo[1,5-a]pyrazine

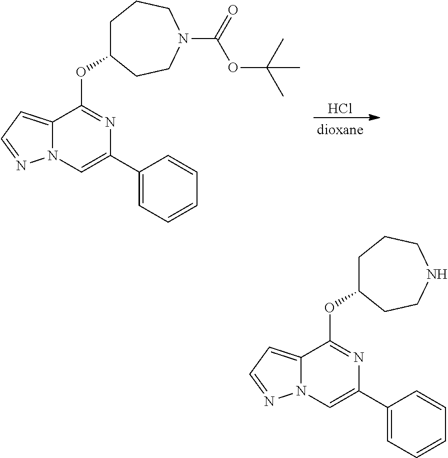 [SMILES: CC(C)(C)OC(=O)N1CCC[C@@H](Oc2nc(-c3ccccc3)cn3nccc23)CC1 ; Cl ; c1ccc(-c2cn3nccc3c(O[C@@H]3CCCNCC3)n2)cc1]

To a solution of tert-butyl (R)-4-((6-phenylpyrazolo[1,5-a]pyrazin-4-yl)oxy)azepane-1-carboxylate (48 mg, 0.12 mmol) in dioxane (1.2 mL) was added HCl (4 M in dioxane, 1.2 mmol, 291 μL). The reaction mixture immediately became a white slurry upon addition of the HCl solution and was stirred at RT for 4 h. The reaction mixture was directly concentrated under reduced pressure to afford crude (R)-4-(azepan-4-yloxy)-6-phenylpyrazolo[1,5-a]pyrazine which was carried forward without further purification assuming 100% yield. LC-MS: m/z=332.2 (M+Na)⁺.

### Synthesis of (R)-1-(4-((6-phenylpyrazolo[1,5-a]pyrazin-4-yl)oxy)azepan-1-yl)prop-2-en-1-one

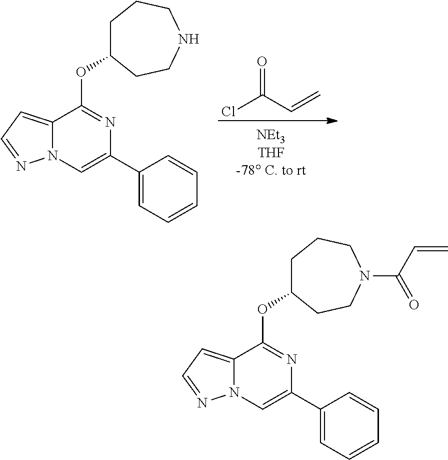

A solution of crude (R)-4-(azepan-4-yloxy)-6-phenylpyrazolo[1,5-a]pyrazine (36 mg, 0.12 mmol) in THF (1.2 mL) was cooled to −78° C. in a dry ice/acetone bath. Triethylamine (81 μL, 0.58 mmol) was added via micro-syringe with stirring, followed immediately by acryloyl chloride (19 μL, 0.23 mmol). The reaction mixture was removed from the ice bath and allowed to slowly warm to RT, becoming red in the process. After stirring at RT for 2 h, the reaction mixture was diluted with EtOAc and quenched by addition of sat. aq. NaHCO₃. The resulting layers were separated, and the aqueous layer was further extracted (2×) with EtOAc. The combined organic extracts were dried over anhydrous sodium sulfate and concentrated under reduced pressure to afford the crude product as a pale-yellow oil. The crude material was purified by reverse-phase HPLC (Column: Waters XSelect CSH Prep C18 5 μm OBD 19×100 mm; Condition: 5-70% Acetonitrile in 0.1% v/v Ammonium carbonate/water) to afford (R)-1-(4-((6-phenylpyrazolo[1,5-a]pyrazin-4-yl)oxy)azepan-1-yl)prop-2-en-1-one (16.7 mg, 40% yield over 2 steps) as a yellow film. LC-MS: m/z=363.3 (M+H)⁺. ¹H NMR (500 MHz, DMSO-d₆) δ ppm 1.71-1.80 (m, 1H) 1.87-2.13 (m, 5H) 2.18-2.30 (m, 1H) 3.52-3.78 (m, 4H) 5.54-5.62 (m, 1H) 5.70 (dt, J=10.38, 2.14 Hz, 1H) 6.15-6.21 (m, 1H) 6.77-6.88 (m, 2H) 7.37-7.42 (m, 1H) 7.45-7.50 (m, 2H) 8.07-8.11 (m, 3H) 9.03 (s, 1H).

### Example 74: (R)-1-(4-((6-(6-methoxypyrimidin-4-yl)pyrazolo[1,5-a]pyrazin-4-yl)oxy)azepan-1-yl)prop-2-en-1-one

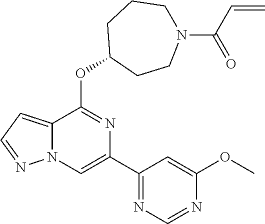 [SMILES: C=CC(=O)N1CCC[C@@H](Oc2nc(-c3cc(OC)ncn3)cn3nccc23)CC1]

### Synthesis of tert-butyl (R)-4-((6-(6-methoxypyrimidin-4-yl)pyrazolo[1,5-a]pyrazin-4-yl)oxy)azepane-1-carboxylate

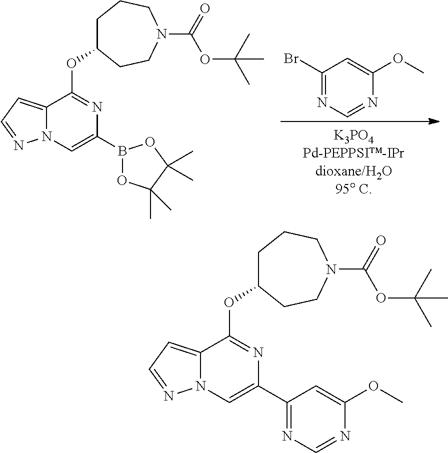

To a 2-dram scintillation vial containing tert-butyl (R)-4-((6-(4,4,5,5-tetramethyl-1,3,2-dioxaborolan-2-yl)pyrazolo[1,5-a]pyrazin-4-yl)oxy)azepane-1-carboxylate (138 mg, 0.30 mmol) in dioxane (3.0 mL) was added 4-bromo-6-methoxypyrimidine (38 mg, 0.20 mmol). A solution of aq. K₃PO₄ (0.5 M, 0.80 mL, 0.40 mmol) was then added, followed by addition of Pd-PEPPSI™-IPr (41 mg, 40 μmol). The reaction mixture was heated at 95° C. overnight, after which time it was cooled to RT and directly concentrated under reduced pressure. The crude material was purified by silica gel chromatography (0 to 50% [3:1 EtOAc:EtOH] in heptanes) to provide tert-butyl (R)-4-((6-(6-methoxypyrimidin-4-yl)pyrazolo[1,5-a]pyrazin-4-yl)oxy)azepane-1-carboxylate (98.6 mg, 75% yield). LC-MS: m/z=441.2 (M+H)⁺.

### Synthesis of (R)-4-(azepan-4-yloxy)-6-(6-methoxypyrimidin-4-yl)pyrazolo[1,5-a]pyrazine

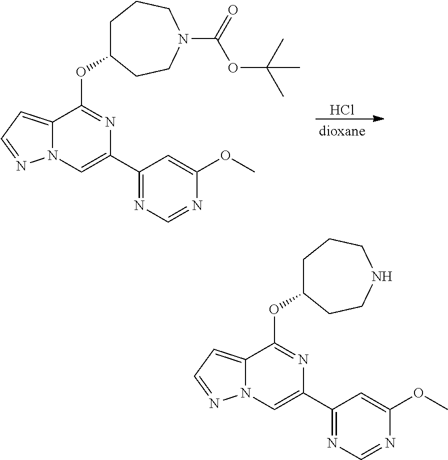 [SMILES: COc1cc(-c2cn3nccc3c(O[C@@H]3CCCN(C(=O)OC(C)(C)C)CC3)n2)ncn1 ; Cl ; COc1cc(-c2cn3nccc3c(O[C@@H]3CCCNCC3)n2)ncn1]

To a solution of tert-butyl (R)-4-((6-(6-methoxypyrimidin-4-yl)pyrazolo[1,5-a]pyrazin-4-yl)oxy)azepane-1-carboxylate (99 mg, 0.22 mmol) in dioxane (2.2 mL) was added HCl (4 M in dioxane, 2.2 mmol, 0.56 mL), forming a milky white slurry. After stirring at RT for 4 h, the reaction mixture was directly concentrated under reduced pressure to afford crude (R)-4-(azepan-4-yloxy)-6-(6-methoxypyrimidin-4-yl)pyrazolo[1,5-a]pyrazine which was used without further purification assuming 100% yield. LC-MS: m/z=363.3 (M+Na)⁺.

### Synthesis of (R)-1-(4-((6-(6-methoxypyrimidin-4-yl)pyrazolo[1,5-a]pyrazin-4-yl)oxy)azepan-1-yl)prop-2-en-1-one

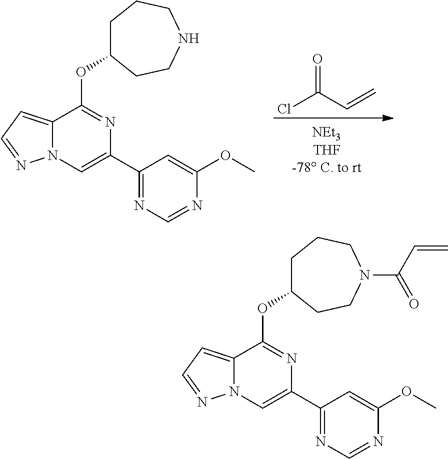

A solution of crude (R)-4-(azepan-4-yloxy)-6-(6-methoxypyrimidin-4-yl)pyrazolo[1,5-a]pyrazine (76 mg, 0.22 mmol) in THF (2.2 mL) was cooled to −78° C. in a dry ice/acetone bath. Triethylamine (156 μL, 1.1 mmol) was added with stirring followed immediately by acryloyl chloride (36 μL, 0.45 mmol). The reaction mixture was removed from the ice bath and allowed to slowly warm to RT, becoming red in the process. After stirring at RT for 2 h, the reaction mixture was diluted with EtOAc and quenched by addition of sat. aq. NaHCO₃. The resulting layers were separated, and the aqueous layer was further extracted (2×) with EtOAc. The combined organic extracts were dried over anhydrous sodium sulfate and concentrated under reduced pressure to afford the crude product as a yellow oil. The crude material was purified via reverse-phase HPLC (Column: Waters XSelect CSH Prep C18 5 μm OBD 19×100 mm; Condition: 5-65% Acetonitrile in 0.1% v/v Ammonium carbonate/water) to afford (R)-1-(4-((6-(6-methoxypyrimidin-4-yl)pyrazolo[1,5-a]pyrazin-4-yl)oxy)azepan-1-yl)prop-2-en-1-one (22.7 mg, 26% yield over 2 steps) as a white solid. LC-MS: m/z=395.3 (M+H)⁺. ¹H NMR (500 MHz, DMSO-d₆) δ ppm 1.71-1.81 (m, 1H) 1.87-1.95 (m, 1H) 1.97 (br s, 1H) 1.98-2.11 (m, 2H) 2.20-2.30 (m, 1H) 3.53-3.81 (m, 4H) 3.99-4.02 (m, 1H) 4.00 (s, 1H) 5.57-5.64 (m, 1H) 5.68-5.72 (m, 1H) 6.18 (dt, J=16.79, 2.59 Hz, 1H) 6.82 (dt, J=16.48, 10.07 Hz, 1H) 6.95-6.98 (m, 1H) 7.53 (dd, J=10.99, 1.22 Hz, 1H) 8.17-8.22 (m, 1H) 8.19-8.19 (m, 1H) 8.19-8.20 (m, 1H) 8.82-8.87 (m, 1H) 8.83-9.11 (m, 1H) 9.09 (s, 1H).

### Example 75: (R)-1-(4-((6-(4-methyloxazol-2-yl)pyrazolo[1,5-a]pyrazin-4-yl)oxy)azepan-1-yl)prop-2-en-1-one

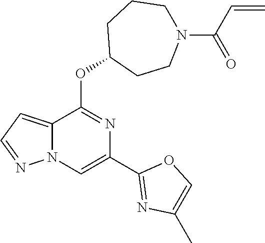 [SMILES: C=CC(=O)N1CCC[C@@H](Oc2nc(-c3nc(C)co3)cn3nccc23)CC1]

### Synthesis of tert-butyl (R)-4-((6-(4-methyloxazol-2-yl)pyrazolo[1,5-a]pyrazin-4-yl)oxy)azepane-1-carboxylate

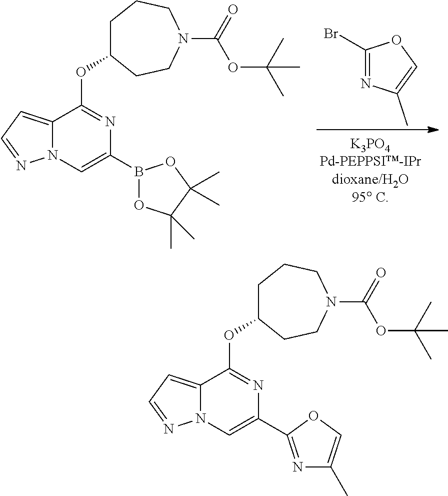

To a 2-dram scintillation vial containing tert-butyl (R)-4-((6-(4,4,5,5-tetramethyl-1,3,2-dioxaborolan-2-yl)pyrazolo[1,5-a]pyrazin-4-yl)oxy)azepane-1-carboxylate (138 mg, 0.30 mmol) in dioxane (3.0 mL) was added 2-bromo-4-methyloxazole (32 mg, 0.20 mmol). Next, a solution of aq. K₃PO₄ (0.5 M, 0.80 mL, 0.40 mmol) and then Pd-PEPPSI™-IPr (41 mg, 40 μmol). The reaction mixture was heated at 95° C. overnight, after which time it was cooled to RT and directly concentrated under reduced pressure. The crude material was purified by silica gel chromatography (0 to 50% [3:1 EtOAc EtOH] in heptanes) to provide tert-butyl (R)-4-((6-(4-methyloxazol-2-yl)pyrazolo[1,5-a]pyrazin-4-yl)oxy)azepane-1-carboxylate (95.4 mg, 77% yield). LC-MS: m/z=441.2 (M+H)⁺.

### Synthesis of (R)-2-(4-(azepan-4-yloxy)pyrazolo[1,5-a]pyrazin-6-yl)-4-methyloxazole

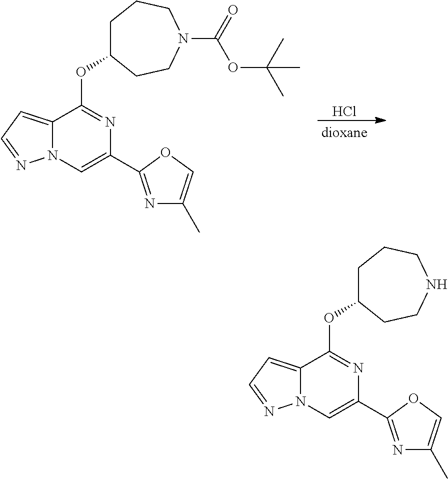 [SMILES: Cc1coc(-c2cn3nccc3c(O[C@@H]3CCCN(C(=O)OC(C)(C)C)CC3)n2)n1 ; Cl ; Cc1coc(-c2cn3nccc3c(O[C@@H]3CCCNCC3)n2)n1]

To a solution of tert-butyl (R)-4-((6-(4-methyloxazol-2-yl)pyrazolo[1,5-a]pyrazin-4-yl)oxy)azepane-1-carboxylate (95 mg, 0.23 mmol) in dioxane (2.3 mL) was added HCl (4 M in dioxane, 2.3 mmol, 0.58 mL), forming a bright yellow slurry. After stirring at RT for 4 h, the reaction mixture was directly concentrated under reduced pressure to afford crude (R)-2-(4-(azepan-4-yloxy)pyrazolo[1,5-a]pyrazin-6-yl)-4-methyloxazole which was used without further purification assuming 100% yield. LC-MS: m/z=314.1 (M+Na)⁺.

### Synthesis of (R)-1-(4-((6-(4-methyloxazol-2-yl)pyrazolo[1,5-a]pyrazin-4-yl)oxy)azepan-1-yl)prop-2-en-1-one

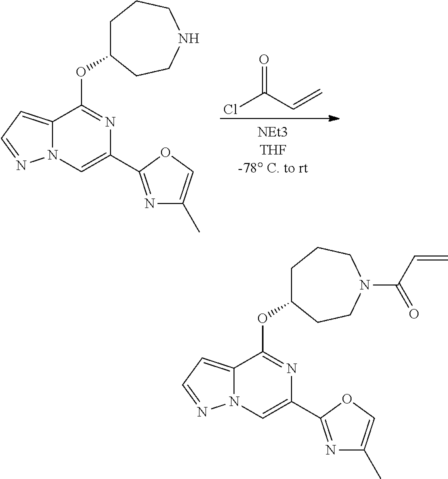

A solution of crude (R)-2-(4-(azepan-4-yloxy)pyrazolo[1,5-a]pyrazin-6-yl)-4-methyloxazole (72 mg, 0.23 mmol) in THF (2.3 mL) was cooled to −78° C. in a dry ice/acetone bath. Triethylamine (161 μL, 1.2 mmol) was added with stirring followed immediately by acryloyl chloride (38 μL, 0.46 mmol). The reaction mixture was removed from the ice bath and allowed to slowly warm to RT, becoming red in the process. After stirring at RT for 2 h, the reaction mixture was diluted with EtOAc and quenched by addition of sat. aq. NaHCO₃. The resulting layers were separated, and the aqueous layer was further extracted (2×) with EtOAc. The combined organic extracts were dried over anhydrous sodium sulfate and concentrated under reduced pressure to afford the crude product as a yellow solid. The crude material was purified via reverse-phase HPLC (Column: Waters XSelect CSH Prep C18 5 μm OBD 19×100 mm; Condition: 5-55% Acetonitrile in 0.1% v/v Ammonium carbonate/water) to afford (R)-1-(4-((6-(4-methyloxazol-2-yl)pyrazolo[1,5-a]pyrazin-4-yl)oxy)azepan-1-yl)prop-2-en-1-one (28.2 mg, 22% yield over 2 steps) as an off-white solid. LC-MS: m/z=368.3 (M+H)⁺. ¹H NMR (500 MHz, DMSO-d₆) δ ppm 1.67-1.79 (m, 1H) 1.83-1.96 (m, 2H) 2.02-2.13 (m, 3H) 2.18 (d, J=1.22 Hz, 4H) 3.54-3.77 (m, 3H) 5.53-5.60 (m, 1H) 5.69 (ddd, J=10.38, 3.66, 2.44 Hz, 1H) 6.13-6.20 (m, 1H) 6.80 (ddd, J=16.48, 14.04, 10.38 Hz, 1H) 6.95-6.98 (m, 1H) 7.97 (s, 1H) 8.18 (d, J=1.22 Hz, 1H) 8.90 (s, 1H).

### Example 76: (R)-1-(4-((6-(2-methylthiazol-4-yl)pyrazolo[1,5-a]pyrazin-4-yl)oxy)azepan-1-yl)prop-2-en-1-one

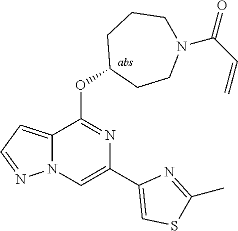 [SMILES: C=CC(=O)N1CCC[C@@H](Oc2nc(-c3csc(C)n3)cn3nccc23)CC1]

### Synthesis of tert-butyl (R)-4-((6-(2-methylthiazol-4-yl)pyrazolo[1,5-a]pyrazin-4-yl)oxy)azepane-1-carboxylate

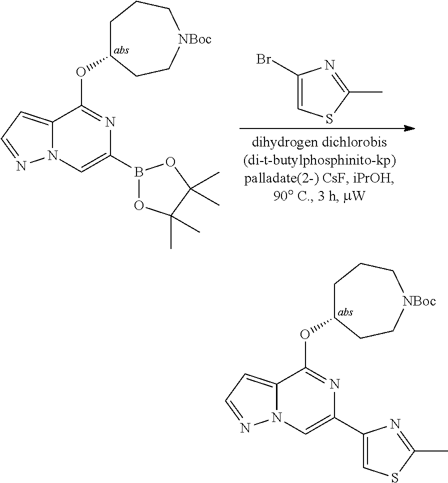

The solution of tert-butyl (R)-4-((6-(4,4,5,5-tetramethyl-1,3,2-dioxaborolan-2-yl)pyrazolo[1,5-a]pyrazin-4-yl)oxy)azepane-1-carboxylate (193 mg, 0.42 mmol), 4-bromo-2-methyl-thiazole (50 mg, 0.28 mmol), dihydrogen dichlorobis(di-tert-butyl phosphinito) palladium (2-) (7 mg, 14 μmol), and cesium fluoride (128 mg, 0.84 mmol) in isopropanol (1.4 mL) was stirred at 90° C. in the microwave for 3 h. The reaction was quenched with water and brine. The biphasic mixture was extracted three times with ethyl acetate then dried over anhydrous MgSO₄. After filtration and concentration under reduced pressure, the crude tert-butyl (R)-4-((6-(2-methylthiazol-4-yl)pyrazolo[1,5-a]pyrazin-4-yl)oxy)azepane-1-carboxylate (110 mg, 91% yield) was taken forward without further purification. LCMS: m/z=430.0 (M+H)⁺.

### Synthesis of (R)-1-(4-((6-(2-methylthiazol-4-yl)pyrazolo[1,5-a]pyrazin-4-yl)oxy)azepan-1-yl)prop-2-en-1-one

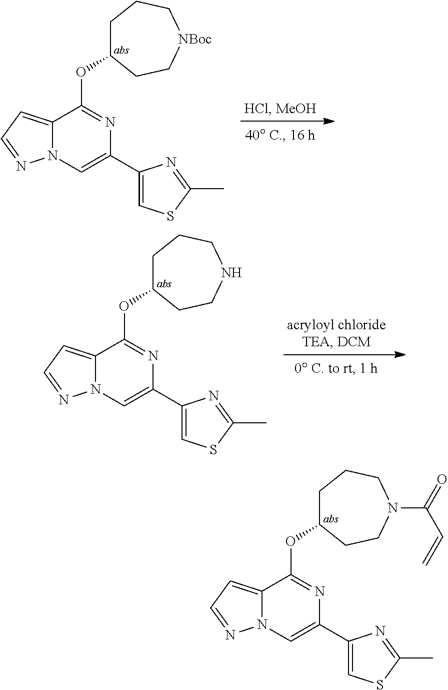

Step 1. The crude tert-butyl (R)-4-((6-(2-methylthiazol-4-yl)pyrazolo[1,5-a]pyrazin-4-yl)oxy)azepane-1-carboxylate (110 mg, 0.26 mmol) was dissolved in an HCl solution (1.25 M in MeOH, 1.5 mL). The reaction solution was stirred at 40° C. After 16 h, the reaction was carefully quenched with slow addition of sat. aq. NaHCO₃ solution. The biphasic mixture was extracted three times with a mixture of chloroform and isopropanol (5:1) then dried over anhydrous MgSO₄. After filtration and concentration under reduced pressure, the crude (R)-4-(4-(azepan-4-yloxy)pyrazolo[1,5-a]pyrazin-6-yl)-2-methylthiazole (84 mg, assumed 100% yield) was concentrated to dryness and taken forward without purification. LCMS: m/z=330.0 (M+H)⁺.

Step 2. To a 20 mL vial containing crude (R)-4-(4-(azepan-4-yloxy)pyrazolo[1,5-a]pyrazin-6-yl)-2-methylthiazole (84 mg, 0.25 mmol) was added DCM (1.0 mL) followed by TEA (129 mg, 1.27 mmol, 178 μL). The reaction mixture was stirred for 5 minutes at room temperature and then was cooled to 0° C. Acryloyl chloride (35 mg, 0.38 mmol, 31 μL) was added dropwise. The solution was stirred at 0° C. After 1 hour, the reaction mixture was carefully quenched with the slow addition of a sat. aq. NH₄Cl solution. The biphasic mixture was extracted three times with ethyl acetate then dried over anhydrous MgSO₄. After filtration and concentration under reduced pressure, the residue was loaded onto a silica gel column and purified (25-100% ethyl acetate in heptanes). The desired fractions were pooled then concentrated under reduced pressure to afford (R)-1-(4-((6-(2-methylthiazol-4-yl)pyrazolo[1,5-a]pyrazin-4-yl)oxy)azepan-1-yl)prop-2-en-1-one (31.2 mg, 32% yield.) ¹H NMR (500 MHz, DMSO-d₆) δ ppm 8.67 (s, 1H) 8.08 (s, 1H) 7.96 (s, 1H) 7.92 (rotamer, s, 1H) 6.77-6.89 (m, 2H) 6.14-6.20 (m, 1H) 5.69 (ddd, J=10.22, 7.48, 2.44 Hz, 1H) 5.52-5.62 (m, 1H) 3.58-3.78 (m, 4H) 2.74 (d, J=1.22 Hz, 3H) 2.23 (ddt, J=10.91, 7.25, 3.43, 3.43 Hz, 1H) 1.87-2.09 (m, 4H) 1.71-1.82 (m, 1H). LCMS m/z=384.0 (M+H)⁺.

### Example 77: (R)-1-(4-((6-(2-methylthiazol-5-yl)pyrazolo[1,5-a]pyrazin-4-yl)oxy)azepan-1-yl)prop-2-en-1-one

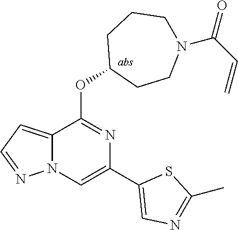 [SMILES: C=CC(=O)N1CCC[C@@H](Oc2nc(-c3cnc(C)s3)cn3nccc23)CC1]

### Synthesis of tert-butyl (R)-4-((6-(2-methylthiazol-5-yl)pyrazolo[1,5-a]pyrazin-4-yl)oxy)azepane-1-carboxylate

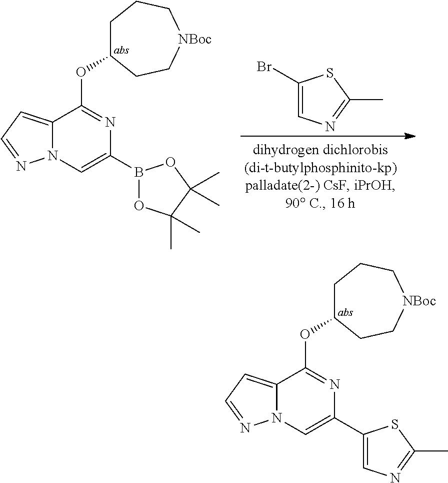

A solution of tert-butyl (R)-4-((6-(4,4,5,5-tetramethyl-1,3,2-dioxaborolan-2-yl)pyrazolo[1,5-a]pyrazin-4-yl)oxy)azepane-1-carboxylate (200 mg, 0.44 mmol), 5-bromo-2-methyl-thiazole (156 mg, 0.87 mmol), dihydrogen dichlorobis(di-tert-butyl phosphinito) palladium (2-) (22 mg, 44 μmol), and cesium fluoride (199 mg, 1.3 mmol) in isopropanol (1.0 mL) was stirred at 90° C. After 16 h, the reaction was quenched with water and brine. The biphasic mixture was extracted three times with ethyl acetate then dried over anhydrous MgSO₄. After filtration and concentration under reduced pressure, the crude tert-butyl (R)-4-((6-(2-methylthiazol-5-yl)pyrazolo[1,5-a]pyrazin-4-yl)oxy)azepane-1-carboxylate (187 mg, assumed 100% yield) was taken forward without further purification. LCMS: m/z=430.0 (M+H)⁺.

### Synthesis of (R)-1-(4-((6-(2-methylthiazol-5-yl)pyrazolo[1,5-a]pyrazin-4-yl)oxy)azepan-1-yl)prop-2-en-1-one

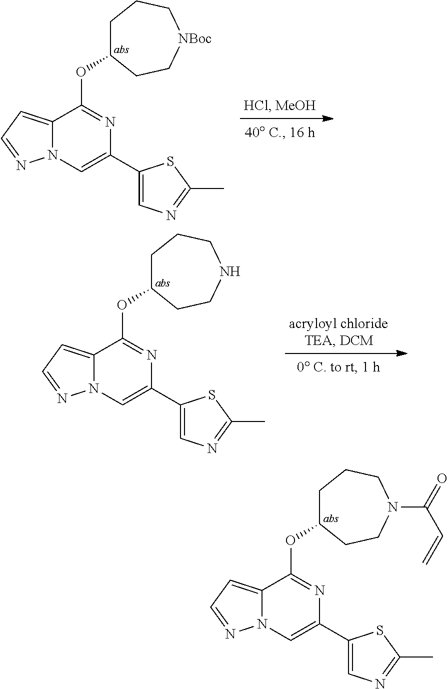

Step 1. The crude tert-butyl (R)-4-((6-(2-methylthiazol-5-yl)pyrazolo[1,5-a]pyrazin-4-yl)oxy)azepane-1-carboxylate (187 mg, 0.44 mmol) was dissolved in an HCl solution (1.25 M in MeOH, 1.7 mL). The reaction solution was stirred at 40° C. After 16 h, the reaction was carefully quenched with slow addition of sat. aq. NaHCO₃ solution. The biphasic mixture was extracted three times with a mixture of chloroform and isopropanol (5:1) then dried over anhydrous MgSO₄. After filtration and concentration under reduced pressure, the crude (R)-5-(4-(azepan-4-yloxy)pyrazolo[1,5-a]pyrazin-6-yl)-2-methylthiazole (143 mg, assumed 100% yield) was concentrated to dryness and taken forward without purification. LCMS: m/z=330.0 (M+H)⁺.

Step 2. To a 20 mL vial containing crude (R)-5-(4-(azepan-4-yloxy)pyrazolo[1,5-a]pyrazin-6-yl)-2-methylthiazole (143 mg, 0.44 mmol) was added DCM (2 mL) followed by TEA (439 mg, 4.34 mmol, 605 μL). The reaction mixture was stirred for 5 minutes at room temperature and then was cooled to 0° C. Acryloyl chloride (79 mg, 0.87 mmol, 71 μL) was added dropwise. The solution was stirred at 0° C. After 1 hour, the reaction mixture was carefully quenched with the slow addition of a sat. aq. NH₄Cl solution. The biphasic mixture was extracted three times with ethyl acetate then dried over anhydrous MgSO₄. After filtration and concentration under reduced pressure, the residue was loaded onto a silica gel column and purified (25-100% ethyl acetate in heptanes). The desired fractions were pooled then concentrated under reduced pressure to afford (R)-1-(4-((6-(2-methylthiazol-5-yl)pyrazolo[1,5-a]pyrazin-4-yl)oxy)azepan-1-yl)prop-2-en-1-one (29.8 mg, 18% yield). ¹H NMR (500 MHz, DMSO-d₆) δ ppm 9.02-9.04 (m, 1H) 8.25 (d, J=2.44 Hz, 1H) 8.09 (dd, J=2.44, 1.22 Hz, 1H) 6.89 (d, J=3.05 Hz, 1H) 6.81 (ddd, J=16.48, 12.82, 10.38 Hz, 1H) 6.17 (ddd, J=16.63, 7.48, 2.75 Hz, 1H) 5.70 (dt, J=10.38, 2.44 Hz, 1H) 5.38-5.44 (m, 1H) 3.55-3.75 (m, 4H) 2.68 (s, 3H) 2.15-2.26 (m, 1H) 1.87-2.10 (m, 4H) 1.68-1.80 (m, 1H). LCMS m/z=384.0 (M+H)⁺.

### Example 78: (R)-1-(4-((6-(3-methylisothiazol-5-yl)pyrazolo[1,5-a]pyrazin-4-yl)oxy)azepan-1-yl)prop-2-en-1-one

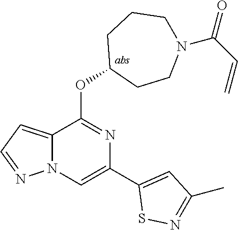 [SMILES: C=CC(=O)N1CCC[C@@H](Oc2nc(-c3cc(C)ns3)cn3nccc23)CC1]

### Synthesis of tert-butyl (R)-4-((6-(3-methylisothiazol-5-yl)pyrazolo[1,5-a]pyrazin-4-yl)oxy)azepane-1-carboxylate

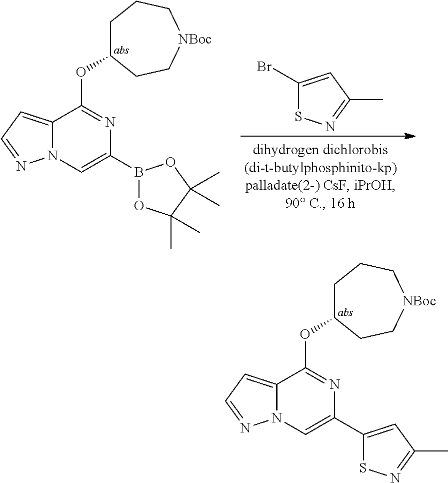

A solution of tert-butyl (R)-4-((6-(4,4,5,5-tetramethyl-1,3,2-dioxaborolan-2-yl)pyrazolo[1,5-a]pyrazin-4-yl)oxy)azepane-1-carboxylate (80 mg, 175 μmol), 5-bromo-3-methyl-isothiazole (47 mg, 262 μmol), dihydrogen dichlorobis(di-tert-butyl phosphinito) palladium (2-) (4.4 mg, 8.7 μmol), and cesium fluoride (80 mg, 524 μmol) in isopropanol (1.0 mL) was stirred at 90° C. After 16 h, the reaction was quenched with water and brine. The biphasic mixture was extracted three times with ethyl acetate then dried over anhydrous MgSO₄. After filtration and concentration under reduced pressure, the crude tert-butyl (R)-4-((6-(3-methylisothiazol-5-yl)pyrazolo[1,5-a]pyrazin-4-yl)oxy)azepane-1-carboxylate (75 mg, assumed 100% yield) was taken forward without further purification. LCMS: m/z=430.0 (M+H)⁺.

### Synthesis of (R)-1-(4-((6-(3-methylisothiazol-5-yl)pyrazolo[1,5-a]pyrazin-4-yl)oxy)azepan-1-yl)prop-2-en-1-one

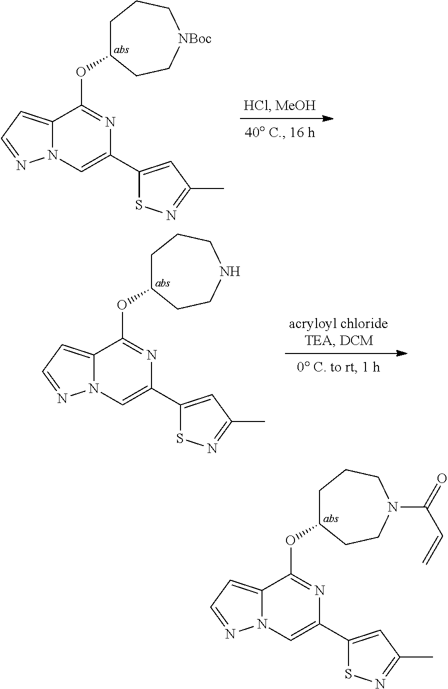

Step 1. The crude tert-butyl (R)-4-((6-(3-methylisothiazol-5-yl)pyrazolo[1,5-a]pyrazin-4-yl)oxy)azepane-1-carboxylate (75 mg, 175 μmol) was dissolved in an HCl solution (1.25 M in MeOH, 1.4 mL). The reaction solution was stirred at 40° C. After 16 h, the reaction was carefully quenched with the slow addition of a sat. aq. NaHCO₃ solution. The biphasic mixture was extracted three times with a mixture of chloroform and isopropanol (5:1) then dried over anhydrous MgSO₄. After filtration and concentration under reduced pressure, the crude (R)-5-(4-(azepan-4-yloxy)pyrazolo[1,5-a]pyrazin-6-yl)-3-methylisothiazole (58 mg, assumed 100% yield) was concentrated to dryness and taken forward without purification. LCMS: m/z=330.0 (M+H)⁺.

Step 2. To a 20 mL vial containing crude (R)-5-(4-(azepan-4-yloxy)pyrazolo[1,5-a]pyrazin-6-yl)-3-methylisothiazole (58 mg, 175 μmol) was added DCM (1.0 mL) followed by TEA (88 mg, 0.87 mmol, 122 μL). The reaction mixture was stirred for 5 minutes at room temperature and then was cooled to 0° C. Acryloyl chloride (24 mg, 262 μmol, 21 μL) was added dropwise. The solution was stirred at 0° C. After 1 hour, the reaction mixture was carefully quenched with the slow addition of a sat. aq. NH₄Cl solution. The biphasic mixture was extracted three times with ethyl acetate then dried over anhydrous MgSO₄. After filtration and concentration under reduced pressure, the residue was loaded onto a silica gel column and purified (25-100% ethyl acetate in heptanes). The desired fractions were pooled then concentrated under reduced pressure to afford (R)-1-(4-((6-(3-methylisothiazol-5-yl)pyrazolo[1,5-a]pyrazin-4-yl)oxy)azepan-1-yl)prop-2-en-1-one (21.3 mg, 32% yield.) ¹H NMR (500 MHz, DMSO-d₆) δ ppm 9.21 (d, J=1.22 Hz, 1H) 8.15 (dd, J=2.44, 1.22 Hz, 1H) 7.80 (d, J=2.44 Hz, 1H) 6.94 (d, J=3.05 Hz, 1H) 6.81 (dt, J=16.63, 10.30 Hz, 1H) 6.14-6.21 (m, 1H) 5.69 (ddd, J=10.38, 5.49, 2.44 Hz, 1H) 5.34-5.43 (m, 1H) 3.65-3.76 (m, 2H) 3.53-3.64 (m, 2H) 2.45 (s, 3H) 2.17-2.29 (m, 1H) 1.95-2.12 (m, 3H) 1.86-1.93 (m, 1H) 1.63-1.83 (m, 1H). LCMS m/z=384.0 (M+H)⁺.

### Example 79: N-methyl-N-((1s,3s)-3-methyl-3-((6-(1-methyl-1H-pyrazol-4-yl)pyrazolo[1,5-a]pyrazin-4-yl)oxy)cyclobutyl)acrylamide

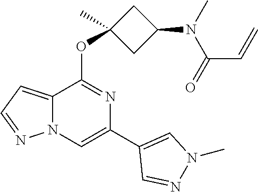 [SMILES: C=CC(=O)N(C)[C@H]1C[C@@](C)(Oc2nc(-c3cnn(C)c3)cn3nccc23)C1]

### Synthesis of tert-butyl methyl((1s,3s)-3-methyl-3-((6-(1-methyl-1H-pyrazol-4-yl)pyrazolo[1,5-a]pyrazin-4-yl)oxy)cyclobutyl)carbamate

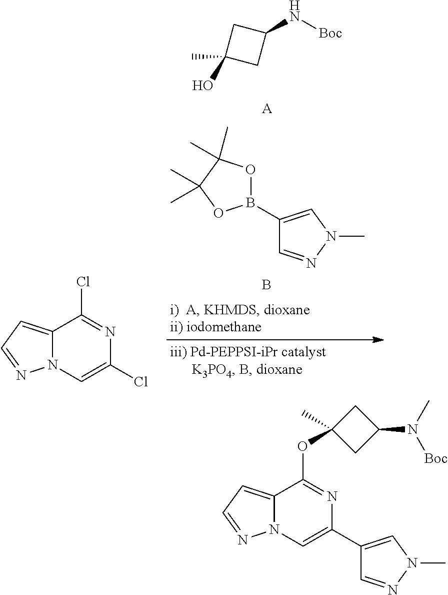

In a 100-mL one-necked round bottom flask equipped with a condenser under a nitrogen atmosphere, potassium hexamethyldisilazide (1 M in THF, 2.2 mL) was added to a solution of tert-butyl ((1s,3s)-3-hydroxy-3-methylcyclobutyl)carbamate (150 mg, 0.75 mmol) in dioxane (7.5 mL) at room temperature. After 5 min, a solution of 4,6-dichloropyrazolo[1,5-a]pyrazine (128 mg, 0.68 mmol) in dioxane (2.5 mL) was dropwise added to the thick white suspension. Iodomethane (240 mg, 1.70 mmol, 105 μL) was added to the resulting orange suspension at room temperature and stirring was continued for an additional 30 min. The resulting reaction mixture was degassed by purging with nitrogen for 30 min, after which a degassed solution of potassium phosphate tribasic (531 mg, 2.50 mmol) in water (2.5 mL) was added at RT. After an additional 10 min of purging of the clear orange reaction mixture with nitrogen, a previously degassed solution of 1-methyl-4-(4,4,5,5-tetramethyl-1,3,2-dioxaborolan-2-yl)pyrazole (212 mg, 1.02 mmol) in dioxane (2.0 mL) was added followed by solid Pd-PEPPSI™-IPr catalyst (93 mg, 0.14 mmol). After purging of the reaction mixture with nitrogen for additional 15 min, the reaction mixture was heated at reflux for 3 hours. To the vigorously stirred reaction mixture were ethyl acetate (20 mL) followed by water (20 mL). After 30 min, the organic phase was separated, and the volatiles were removed under reduced pressure. The resulting residue was purified by column chromatography (40 g silica gel, 0-80% [3:1 EtOAc:EtOH] with 2% NH₄OH modifier in heptanes) yielding the title compound as a pale yellow oil (130 mg, 47% yield). LCMS m/z=413.1 (M+H)⁺. ¹H NMR (500 MHz, methanol-d₄) δ ppm 8.42 (s, 1H), 8.05 (s, 1H), 7.93 (s, 1H), 7.91 (d, J=2.44 Hz, 1H), 6.77 (d, J=1.22 Hz, 1H), 4.10-4.45 (m, 1H), 3.95 (s, 3H), 2.82 (s, 3H), 2.74-2.81 (m, 2H), 2.67 (br s, 2H), 1.81 (s, 3H), 1.46 (s, 9H).

### Synthesis of (1s,3s)-N,3-dimethyl-3-((6-(1-methyl-1H-pyrazol-4-yl)pyrazolo[1,5-a]pyrazin-4-yl)oxy)cyclobutan-1-amine

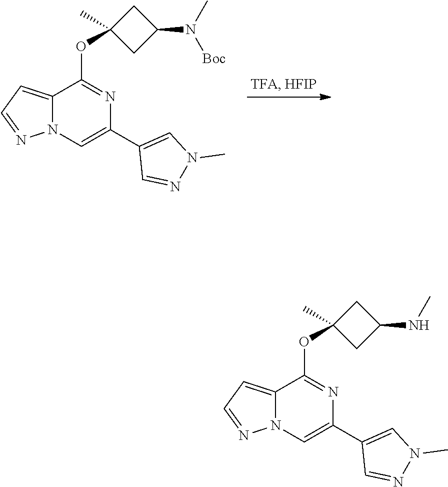 [SMILES: Cn1cc(-c2cn3nccc3c(O[C@]3(C)C[C@H](N(C)C(=O)OC(C)(C)C)C3)n2)cn1 ; CN[C@H]1C[C@@](C)(Oc2nc(-c3cnn(C)c3)cn3nccc23)C1]

To a solution of tert-butyl methyl((1s,3s)-3-methyl-3-((6-(1-methyl-1H-pyrazol-4-yl)pyrazolo[1,5-a]pyrazin-4-yl)oxy)cyclobutyl)carbamate (4.05 g, 9.82 mmol) in HFIP (45 mL) was added TFA (2.24 g, 19.6 mmol, 1.5 mL) at room temperature. The resulting reaction mixture was stirred overnight. Ethyl acetate (50 mL) was added followed by a sat. aq. NaHCO₃ solution (25 mL) and brine (10 mL). After vigorous stirring for 30 min, the organic phase was separated, dried over sodium sulfate, filtered, and concentrated. The resulting residue was purified by column chromatography (24 g silica gel, 80-100% [3:1 EtOAc:EtOH] with 2% NH₄OH modifier in heptanes) yielding the title compound as a pale yellow gum (2.53 g, 82% yield). LCMS m/z=313.1 (M+H)⁺. ¹H NMR (500 MHz, methanol-d₄) δ ppm 8.41 (d, J=1.22 Hz, 1H), 8.05 (s, 1H), 7.86-7.97 (m, 2H), 6.72-6.81 (m, 1H), 3.95 (s, 3H), 2.96-3.11 (m, 1H), 2.76-2.90 (m, 2H), 2.31 (s, 3H), 2.25-2.31 (m, 2H), 2.25-2.31 (m, 2H), 1.80 (s, 3H).

### Synthesis of N-methyl-N—((1s,3s)-3-methyl-3-((6-(1-methyl-1H-pyrazol-4-yl)pyrazolo[1,5-a]pyrazin-4-yl)oxy)cyclobutyl)acrylamide

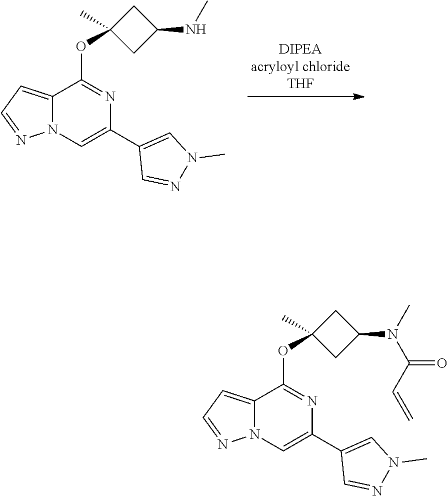 [SMILES: CN[C@H]1C[C@@](C)(Oc2nc(-c3cnn(C)c3)cn3nccc23)C1 ; C=CC(=O)N(C)[C@H]1C[C@@](C)(Oc2nc(-c3cnn(C)c3)cn3nccc23)C1]

To a solution of N-methyl-N-((1s,3s)-3-methyl-3-((6-(1-methyl-1H-pyrazol-4-yl)pyrazolo[1,5-a]pyrazin-4-yl)oxy)cyclobutyl)acrylamide (4.05 g, 9.82 mmol) and DIPEA (2.81 g, 21.7 mmol, 3.8 mL) in THF (50 mL) was added acryloyl chloride (819 mg, 9.04 mmol, 740 μL) at 0° C. After 30 min, the reaction mixture was diluted with EtOAc (50 mL) and a sat. aq. NaHCO₃ solution (50 mL) were added. The vigorously stirred biphasic mixture was brought to room temperature and stirring was continued for another 30 min. The organic phase was separated, washed with water (25 mL) and brine (25 mL), dried over Na₂SO₄, filtered, and concentrated. The resulting residue was purified by column chromatography (80 g silica gel, 0-100% [3:1 EtOAc:EtOH] with 2% NH₄OH modifier in heptanes). The colorless solid was recrystallized from EtOAc/heptanes (1/3, 45 mL) to afford the title compound as a free flowing crystalline solid (1.8 g, 68% yield). Melting point=137.5° C. LCMS m/z=389.1.1 (M+Na)⁺. ¹H NMR (500 MHz, methanol-d₄) δ ppm 8.44 (s, 1H), 8.06 (s, 1H), 7.85-7.98 (m, 2H), 6.67-6.85 (m, 2H), 6.12-6.26 (m, 1H), 5.74 (br d, J=9.16 Hz, 1H), 4.45-4.77 (m, 1H), 3.95 (s, 3H), 2.94-3.12 (m, 3H), 2.62-2.94 (m, 4H), 1.86 (s, 3H).

### Example 80: N-((1s,3s)-3-((6-(1H-pyrazol-4-yl)pyrazolo[1,5-a]pyrazin-4-yl)oxy)-3-methylcyclobutyl)-N-methylacrylamide

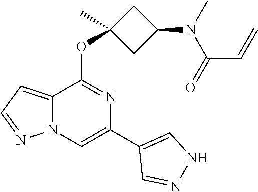 [SMILES: C=CC(=O)N(C)[C@H]1C[C@@](C)(Oc2nc(-c3cn[nH]c3)cn3nccc23)C1]

### Synthesis of tert-butyl ((1s,3s)-3-((6-chloropyrazolo[1,5-a]pyrazin-4-yl)oxy)-3-methylcyclobutyl)(methyl)carbamate

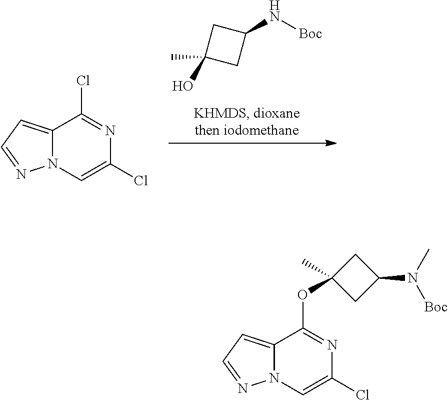 [SMILES: Clc1cn2nccc2c(Cl)n1 ; CC(C)(C)OC(=O)N[C@H]1C[C@@](C)(O)C1 ; CN(C(=O)OC(C)(C)C)[C@H]1C[C@@](C)(Oc2nc(Cl)cn3nccc23)C1]

In a 100-mL one-necked round bottom flask equipped with a condenser under a nitrogen atmosphere, potassium hexamethyldisilazide (1 M in THF, 6.8 mL) was added to a solution of tert-butyl ((1s,3s)-3-hydroxy-3-methylcyclobutyl)carbamate (500 mg, 2.48 mmol) in dioxane (25 mL) at room temperature. After an additional 15 min, a solution of 4,6-dichloropyrazolo[1,5-a]pyrazine (425 mg, 2.26 mmol) in dioxane (7.5 mL) was added dropwise to the thick white suspension. After additional 30 min, iodomethane (240 mg, 1.70 mmol, 105 μL) was added dropwise to the resulting orange suspension at room temperature and stirring was continued for 30 min. The reaction mixture was diluted with EtOAc (40 mL) and washed with water (30 mL). The organic phase was separated, concentrated under reduced pressure, and purified by column chromatography (40 g silica gel, 0-80% [3:1 EtOAc:EtOH] with 2% NH₄OH modifier in heptanes) to afford the title compound as a beige solid (555 mg, 67% yield). LCMS m/z=367.1 (M+H)⁺.

### Synthesis of tert-butyl 4-(4-((1s,3s)-3-((tert-butoxycarbonyl)(methyl)amino)-1-methylcyclobutoxy)pyrazolo[1,5-a]pyrazin-6-yl)-1H-pyrazole-1-carboxylate

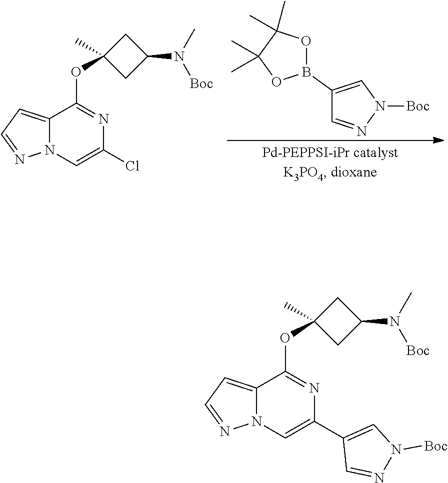

To a solution of tert-butyl ((1s,3s)-3-((6-chloropyrazolo[1,5-a]pyrazin-4-yl)oxy)-3-methylcyclobutyl)(methyl)carbamate (500 mg, 1.36 mmol) and tert-butyl 4-(4,4,5,5-tetramethyl-1,3,2-dioxaborolan-2-yl)pyrazole-1-carboxylate (802 mg, 2.73 mmol) in dioxane (15 mL) were sequentially added Pd-PEPPSI™-IPr catalyst (186 mg, 0.27 mmol), potassium phosphate tribasic (579 mg, 2.73 mmol) and water (3 mL). The resulting mixture was degassed by purging with nitrogen for 30 min. After heating at reflux for 1 h, the reaction mixture was cooled to room temperature and EtOAc (20 mL) and water (20 mL) were added. After vigorous stirring for 30 min, the organic phase was separated, washed with brine (20 mL), dried over Na₂SO₄, and concentrated under reduced pressure. Purification of the crude residue by column chromatography (40 g silica gel, 0-60% [3:1 EtOAc:EtOH] with 2% NH₄OH modifier in heptanes) afforded the title compound as an orange gum (640 mg, 94% yield). LCMS m/z=499.2 (M+H)+.

### Synthesis of (1s,3s)-3-((6-(1H-pyrazol-4-yl)pyrazolo[1,5-a]pyrazin-4-yl)oxy)-N,3-dimethylcyclobutan-1-amine

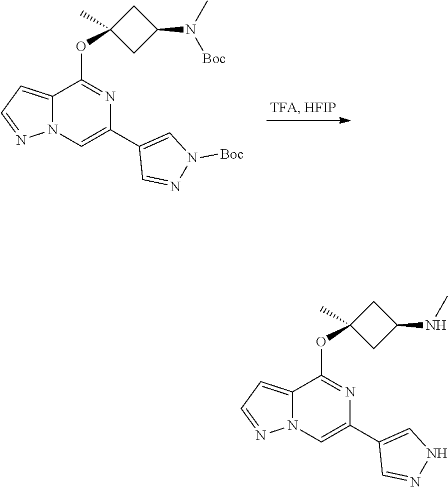 [SMILES: CN(C(=O)OC(C)(C)C)[C@H]1C[C@@](C)(Oc2nc(-c3cnn(C(=O)OC(C)(C)C)c3)cn3nccc23)C1 ; CN[C@H]1C[C@@](C)(Oc2nc(-c3cn[nH]c3)cn3nccc23)C1]

To a solution of tert-butyl 4-(4-((1s,3s)-3-((tert-butoxycarbonyl)(methyl)amino)-1-methylcyclobutoxy)pyrazolo[1,5-a]pyrazin-6-yl)-1H-pyrazole-1-carboxylate (360 mg, 0.72 mmol) in HFIP (5 mL) was added TFA (374 mg, 3.3 mmol, 250 μL) at room temperature. The resulting reaction mixture stirred for 2 hours. Ethyl acetate (20 mL) was added at room temperature followed by a sat. aq. NaHCO₃ solution (10 mL) and brine (10 mL). After vigorous stirring for 30 min, the organic phase was separated, dried over sodium sulfate, filtered, and concentrated. The resulting residue was purified by column chromatography (12 g silica gel, 80-100% [3:1 EtOAc:EtOH] with 2% NH₄OH modifier in heptanes) yielding the title compound as a colorless gum (162 mg, 75% yield). LCMS m/z=299.0 (M+H)+.

### Synthesis of N—((1s,3s)-3-((6-(1H-pyrazol-4-yl)pyrazolo[1,5-a]pyrazin-4-yl)oxy)-3-methylcyclobutyl)-N-methylacrylamide

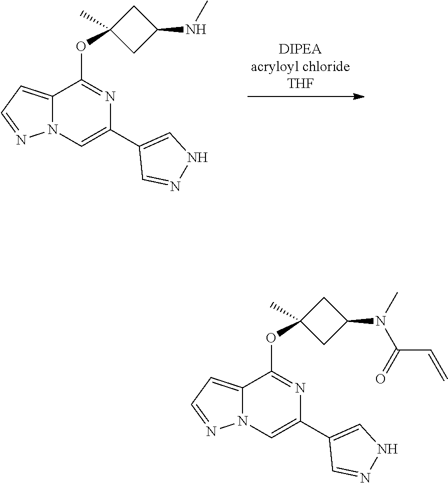 [SMILES: CN[C@H]1C[C@@](C)(Oc2nc(-c3cn[nH]c3)cn3nccc23)C1 ; C=CC(=O)N(C)[C@H]1C[C@@](C)(Oc2nc(-c3cn[nH]c3)cn3nccc23)C1]

To a solution of (1s,3s)-3-((6-(1H-pyrazol-4-yl)pyrazolo[1,5-a]pyrazin-4-yl)oxy)-N,3-dimethylcyclobutan-1-amine (162 mg, 0.54 mmol) and DIPEA (211 mg, 1.63 mmol, 290 μL) in THF (5 mL) was added acryloyl chloride (54 mg, 0.60 mmol, 50 μL) at 0° C. After 30 min, the reaction mixture was diluted with EtOAc (20 mL) and a sat. aq. NaHCO₃ solution (20 mL) were added. The biphasic mixture was brought to room temperature and vigorous stirring was continued for another 30 min. The organic phase was separated, washed sequentially with water (10 mL) and brine (10 mL), dried over Na₂SO₄, filtered, and concentrated. The resulting residue was purified by column chromatography (12 g silica gel, 0-100% [3:1 EtOAc:EtOH] with 2% NH₄OH modifier in heptanes) affording the title compound as a colorless solid (65 mg, 34% yield). LCMS m/z=375.1 (M+Na)+. ¹H NMR (500 MHz, methanol-d₄) δ ppm 8.39-8.48 (m, 1H), 8.07 (br s, 2H), 7.83-7.95 (m, 1H), 6.62-6.86 (m, 2H), 6.08-6.27 (m, 1H), 5.56-5.83 (m, 1H), 4.03-4.75 (m, 1H), 2.88-3.09 (m, 3H), 2.44-2.88 (m, 4H), 1.83 (m, 3H).

### Example 81. 1-(3-(2-((6-(1-methyl-1H-pyrazol-4-yl)pyrazolo[1,5-a]pyridin-4-yl)oxy)ethyl)morpholino)prop-2-en-1-one

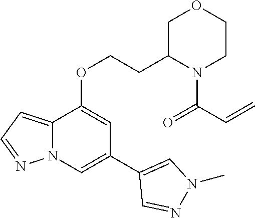 [SMILES: C=CC(=O)N1CCOCC1CCOc1cc(-c2cnn(C)c2)cn2nccc12]

### 1. Synthesis of tert-butyl 3-(2-((methylsulfonyl)oxy)ethyl)morpholine-4-carboxylate

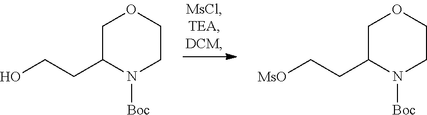 [SMILES: CC(C)(C)OC(=O)N1CCOCC1CCO ; CC(C)(C)OC(=O)N1CCOCC1CCOS(C)(=O)=O]

TEA (1.1 equiv.) was added to a solution of 6-(1-methyl-1H-pyrazol-4-yl)pyrazolo[1,5-a]pyridin-4-ol (1.0 equiv.) in dry DCM (10 mL) followed by mesyl chloride (1.05 equiv.) and the reaction mixture was stirred for 14 h. The mixture was washed with H₂O (10 mL), dried over Na₂SO₄, filtered and concentrated in vacuo, to give crude product which was used directly in the next step

### 2. Synthesis of tert-butyl 3-(2-((6-(1-methyl-1H-pyrazol-4-yl)pyrazolo[1,5-a]pyridin-4-yl)oxy)ethyl)morpholine-4-carboxylate

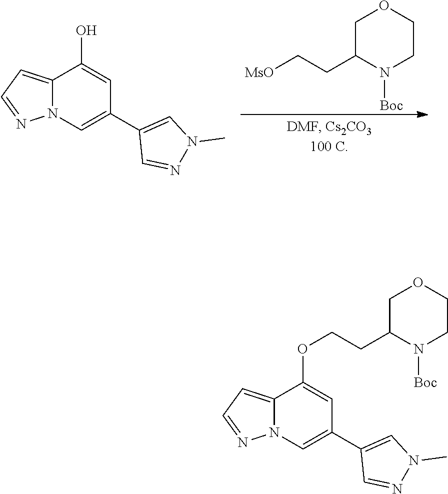 [SMILES: Cn1cc(-c2cc(O)c3ccnn3c2)cn1 ; CC(C)(C)OC(=O)N1CCOCC1CCOS(C)(=O)=O ; Cn1cc(-c2cc(OCCC3COCCN3C(=O)OC(C)(C)C)c3ccnn3c2)cn1]

A mixture of 6-(1-methyl-1H-pyrazol-4-yl)pyrazolo[1,5-a]pyridin-4-ol (Intermediate C, step 2, 1.0 equiv.), Cs₂CO₃ (1.1 equiv.), tert-butyl 3-(2-((methylsulfonyl)oxy)ethyl) morpholine-4-carboxylate (1.0 equiv.) in dry DMF (1 mL) was heated at 100° C. for 16 h under Ar (g). The reaction mixture was diluted with H₂O (10 mL) and extracted with EtOAc (3×10 mL). The combined organic layers were washed with brine (10 mL), dried over Na₂SO₄, filtered and concentrated in vacuo to give crude product which was used directly in the next step.

### 3. Synthesis of 3-(2-((6-(1-methyl-1H-pyrazol-4-yl)pyrazolo[1,5-a]pyridin-4-yl)oxy)ethyl)morpholine hydrochloride

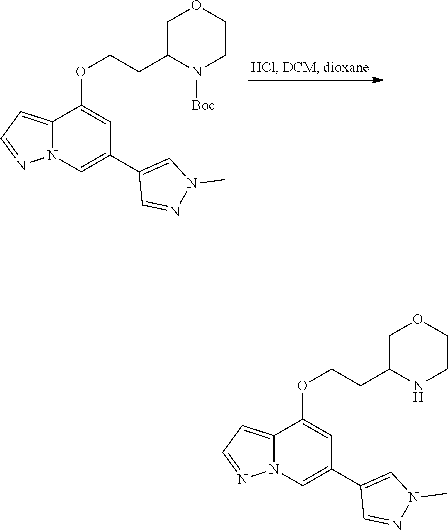 [SMILES: Cn1cc(-c2cc(OCCC3COCCN3C(=O)OC(C)(C)C)c3ccnn3c2)cn1 ; Cn1cc(-c2cc(OCCC3COCCN3)c3ccnn3c2)cn1]

To a solution of tert-butyl 3-(2-((6-(1-methyl-1H-pyrazol-4-yl)pyrazolo[1,5-a]pyridin-4-yl)oxy)ethyl)morpholine-4-carboxylate (1 equiv.) in DCM (10 mL) was added 4M HCl in dioxane (10 eq.) and the resulting solution was stirred for 14 h at 25° C. The reaction mixture was concentrated under reduced pressure. The product was collected by filtration, washed with IPA (3×10 mL), and then dried in vacuo at 40° C. to give 3-(2-((6-(1-methyl-1H-pyrazol-4-yl)pyrazolo[1,5-a]pyridin-4-yl)oxy)ethyl)morpholine hydrochloride.

### 4. Synthesis of 1-(3-(2-((6-(1-methyl-1H-pyrazol-4-yl)pyrazolo[1,5-a]pyridin-4-yl)oxy)ethyl)morpholino)prop-2-en-1-one

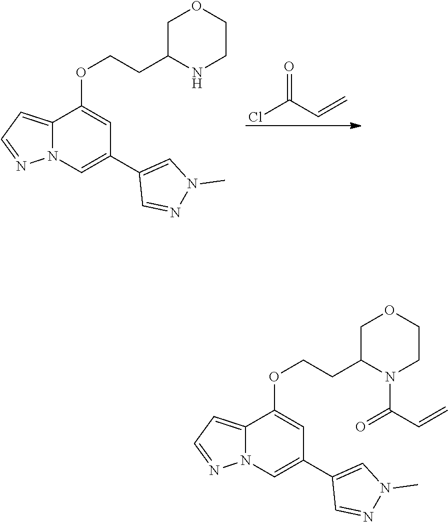

To a solution of 3-(2-((6-(1-methyl-1H-pyrazol-4-yl)pyrazolo[1,5-a]pyridin-4-yl)oxy)ethyl)morpholine hydrochloride (1 equiv.) in DCM (10 mL) was added DIPEA (1.1 equiv.) the mixture was cooled to −10° C., acryloyl chloride (1.05 equiv.) added and the reaction was stirred at RT for 3 h. The reaction mixture was washed with water (10 mL), dried over Na₂SO₄, filtered and was concentrated in vacuo. The crude product was dissolved in DMSO (0.5 mL) and purified by prep. HPLC (Waters SunFire C18 19*100 5 mkm column) to afford 1-(3-(2-((6-(1-methyl-1H-pyrazol-4-yl)pyrazolo[1,5-a]pyridin-4-yl)oxy)ethyl)morpholino)prop-2-en-1-one, 10.2 mg. LCMS m/z=382.2 (M+H)+. 1H NMR (400 MHz, CDCl₃) δ ppm: 8.43-8.25 (m, 1H), 7.86 (s, 1H), 7.70 (s, 1H), 7.64-7.55 (m, 1H), 6.65-6.39 (m, 3H), 6.21 (dd, J=16.7, 1.8 Hz, 1H), 5.68-4.43 (m, 2H), 4.30-4.03 (m, 2H), 4.01-3.83 (m, 5H), 3.72-3.01 (m, 3H), 2.55-2.45 (m, 1H), 2.32-2.25 (m, 2H)

### Example 82. N-(5-((6-(1-methyl-1H-pyrazol-4-yl)pyrazolo[1,5-a]pyridin-4-yl)oxy)bicyclo[2.2.1]heptan-2-yl)acrylamide

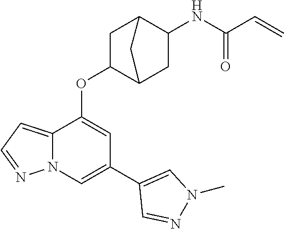 [SMILES: C=CC(=O)NC1CC2CC1CC2Oc1cc(-c2cnn(C)c2)cn2nccc12]

N-(5-((6-(1-Methyl-1H-pyrazol-4-yl)pyrazolo[1,5-a]pyridin-4-yl)oxy)bicyclo[2.2.1]heptan-2-yl)acrylamide was obtained from 6-(1-methyl-1H-pyrazol-4-yl)pyrazolo[1,5-a]pyridin-4-ol (Intermediate C, step 2) and tert-butyl (5-hydroxybicyclo[2.2.1]heptan-2-yl)carbamate, following the steps described in Example 81. LCMS m/z=378.2 (M+H)+. 1H NMR (400 MHz, CDCl₃) δ ppm: 8.27 (s, 1H), 7.89 (d, J=2.4 Hz, 1H), 7.72 (s, 1H), 7.59 (s, 1H), 6.58 (d, J=2.5 Hz, 1H), 6.43 (s, 1H), 6.25 (d, J=16.8 Hz, 1H), 6.07 (dd, J=17.0, 10.2 Hz, 1H), 5.83 (d, J=7.3 Hz, 1H), 5.61 (d, J=10.3 Hz, 1H), 4.91-4.83 (m, 1H), 4.47-4.38 (m, 1H), 3.98 (s, 3H), 2.78-2.72 (m, 1H), 2.72-2.65 (m, 1H), 2.07 (t, J=13.3, 13.3 Hz, 2H), 1.73-1.59 (m, 4H)

### Example 83. (R)-1-(2,2-dimethyl-6-(((6-(1-methyl-1H-pyrazol-4-yl)pyrazolo[1,5-a]pyridin-4-yl)oxy)methyl)morpholino)prop-2-en-1-one

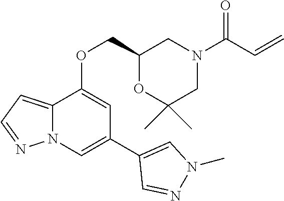 [SMILES: C=CC(=O)N1C[C@H](COc2cc(-c3cnn(C)c3)cn3nccc23)OC(C)(C)C1]

(R)-1-(2,2-dimethyl-6-(((6-(1-methyl-1H-pyrazol-4-yl)pyrazolo[1,5-a]pyridin-4-yl)oxy)methyl)morpholino)prop-2-en-1-one was obtained from tert-butyl (R)-6-(hydroxymethyl)-2,2-dimethylmorpholine-4-carboxylate and 6-(1-methyl-1H-pyrazol-4-yl)pyrazolo[1,5-a]pyridin-4-ol (Intermediate C, step 2), following a similar method to that described in Example 81. LCMS m/z=396.2 (M+H)+. 1H NMR (400 MHz, MeOH-d₄) δ ppm: 8.36 (s, 1H), 8.01 (s, 1H), 7.88 (s, 1H), 7.86 (d, J=2.4 Hz, 1H), 6.91-6.72 (m, 2H), 6.66 (s, 1H), 6.29 (dd, J=16.9, 8.1 Hz, 1H), 5.81 (dd, J=11.0, 5.6 Hz, 1H), 4.54 (dd, J=142.3, 13.0 Hz, 1H), 4.29-4.17 (m, 3.5H), 3.97-3.90 (m, 3.5H), 3.28-3.08 (m, 1H), 2.86-2.69 (m, 1H), 1.32-1.23 (m, 6H)

### Examples 84 and 85. 1-((1R,5S,6s)-6-(((3-fluoro-6-(1-methyl-1H-pyrazol-4-yl)pyrazolo[1,5-a]pyridin-4-yl)oxy)methyl)-3-azabicyclo[3.1.1]heptan-3-yl)prop-2-en-1-one and 1-((1R,5S,6r)-6-(((3-fluoro-6-(1-methyl-1H-pyrazol-4-yl)pyrazolo[1,5-a]pyridin-4-yl)oxy)methyl)-3-azabicyclo[3.1.1]heptan-3-yl)prop-2-en-1-one

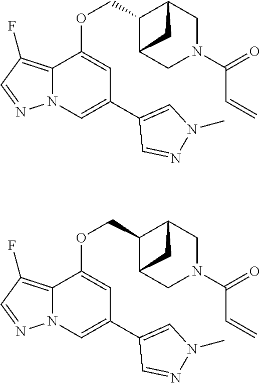 [SMILES: C=CC(=O)N1C[C@H]2C[C@@H](C1)[C@H]2COc1cc(-c2cnn(C)c2)cn2ncc(F)c12 ; C=CC(=O)N1C[C@H]2C[C@@H](C1)[C@@H]2COc1cc(-c2cnn(C)c2)cn2ncc(F)c12]

### 1. Synthesis of tert-butyl 6-(((methylsulfonyl)oxy)methyl)-3-azabicyclo[3.1.1]heptane-3-carboxylate

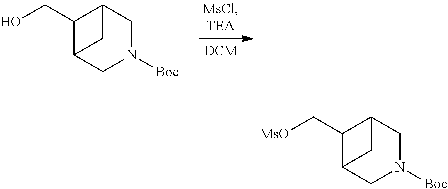 [SMILES: CC(C)(C)OC(=O)N1CC2CC(C1)C2CO ; CC(C)(C)OC(=O)N1CC2CC(C1)C2COS(C)(=O)=O]

To a solution of tert-butyl 6-(hydroxymethyl)-3-azabicyclo[3.1.1]heptane-3-carboxylate (1 g, 4.40 mmol) and TEA (1.34 g, 13.2 mmol) in DCM (20 mL) was added methanesulfonyl chloride (0.72 g, 6.29 mmol) and the reaction was stirred at 0° C. for 1 h. Water (10 mL) was added and the mixture was extracted with DCM (20 mL×3). The combined organics were washed with brine (20 mL), dried over Na₂SO₄, filtered and concentrated in vacuo to give tert-butyl 6-(((methylsulfonyl)oxy)methyl)-3-azabicyclo[3.1.1]heptane-3-carboxylate (1.7 g, crude) as yellow oil. 1H NMR: (500 MHz, DMSO-d₆) δ: 4.45 (d, J=8.0 Hz, 1H), 4.16 (d, J=7.5 Hz, 1H), 3.57-3.31 (m, 5H), 3.18 (d, J=14.5 Hz, 3H), 2.50-2.35 (m, 2H), 2.31-2.22 (m, 1H), 2.04-1.95 (m, 1H), 1.41 (s, 9H).

### 2. Synthesis of tert-butyl 6-(((6-bromo-3-fluoropyrazolo[1,5-a]pyridin-4-yl)oxy)methyl)-3-azabicyclo[3.1.1]heptane-3-carboxylate

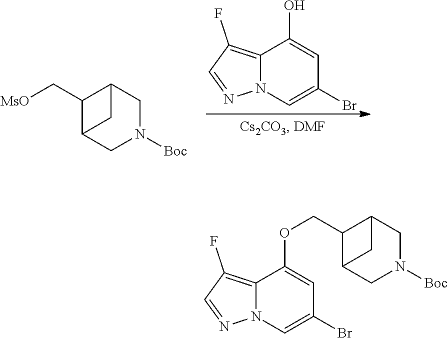 [SMILES: CC(C)(C)OC(=O)N1CC2CC(C1)C2COS(C)(=O)=O ; Oc1cc(Br)cn2ncc(F)c12 ; CC(C)(C)OC(=O)N1CC2CC(C1)C2COc1cc(Br)cn2ncc(F)c12]

To a solution of 6-bromo-3-fluoropyrazolo[1,5-a]pyridin-4-ol (80 mg, 346 μmol) in DMF (4 mL) was added Cs₂CO₃ (200 mg, 614 μmol) and tert-butyl 6-(((methylsulfonyl)oxy)methyl)-3-azabicyclo[3.1.1]heptane-3-carboxylate (212 mg, 693 μmol) and the reaction stirred at 100° C. for 1 h. The reaction mixture was concentrated in vacuo and the crude product was purified by prep-TLC (PE: EtOAc=3:1) to give tert-butyl 6-(((6-bromo-3-fluoropyrazolo[1,5-a]pyridin-4-yl)oxy)methyl)-3-azabicyclo[3.1.1]heptane-3-carboxylate (100 mg, 56% yield) as brown oil. LCMS m/z=384.2 (M+H)+

### 3. Synthesis of tert-butyl 6-(((3-fluoro-6-(1-methyl-1H-pyrazol-4-yl)pyrazolo[1,5-a]pyridin-4-yl)oxy)methyl)-3-azabicyclo[3.1.1]heptane-3-carboxylate

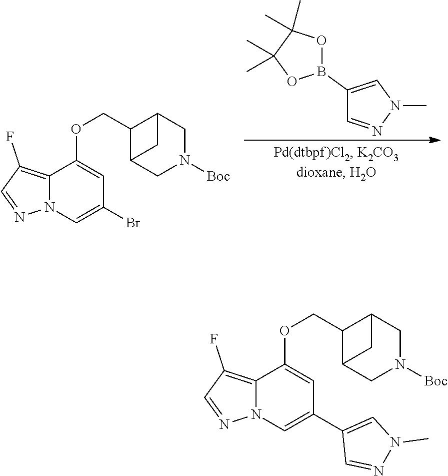

A mixture of tert-butyl 6-(((6-bromo-3-fluoropyrazolo[1,5-a]pyridin-4-yl)oxy)methyl)-3-azabicyclo[3.1.1]heptane-3-carboxylate (90 mg, 204 μmol), 1-methyl-4-(4,4,5,5-tetramethyl-1,3,2-dioxaborolan-2-yl)-1H-pyrazole (60 mg, 288 μmol), K₂CO₃ (85 mg, 613 μmol) and Pd(dtbpf)Cl₂ (13 mg, 20 μmol) in dioxane (5 mL) and water (1 mL) was purged with N₂ for 1 min and the reaction was stirred at 90° C. for 2 h. The reaction mixture was concentrated in vacuum and the crude product was purified by Combiflash® eluting with EtOAc in PE from 0% to 100% to give tert-butyl 6-(((3-fluoro-6-(1-methyl-1H-pyrazol-4-yl)pyrazolo[1,5-a]pyridin-4-yl)oxy)methyl)-3-azabicyclo[3.1.1]heptane-3-carboxylate (90 mg, 90% yield) as yellow oil. LCMS m/z=442.3 (M+H)+

### 4. Synthesis of (1R,5S,6s)-6-(((3-fluoro-6-(1-methyl-1H-pyrazol-4-yl)pyrazolo[1,5-a]pyridin-4-yl)oxy)methyl)-3-azabicyclo[3.1.1]heptane and (1R,5S,6r)-6-(((3-fluoro-6-(1-methyl-1H-pyrazol-4-yl)pyrazolo[1,5-a]pyridin-4-yl)oxy)methyl)-3-azabicyclo[3.1.1]heptane

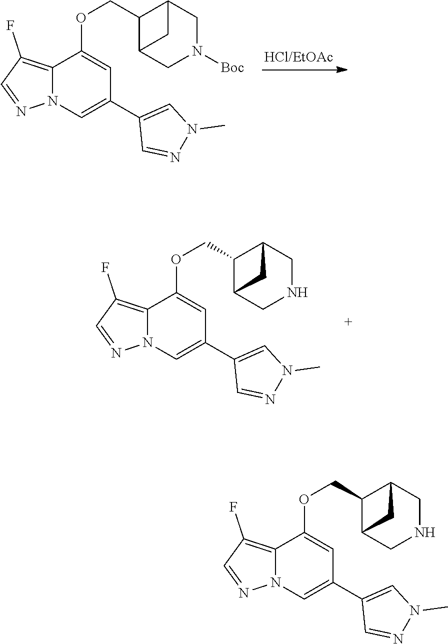 [SMILES: Cn1cc(-c2cc(OCC3C4CC3CN(C(=O)OC(C)(C)C)C4)c3c(F)cnn3c2)cn1 ; Cn1cc(-c2cc(OC[C@@H]3[C@@H]4CNC[C@H]3C4)c3c(F)cnn3c2)cn1 ; Cn1cc(-c2cc(OC[C@H]3[C@@H]4CNC[C@H]3C4)c3c(F)cnn3c2)cn1]

To a solution of tert-butyl 6-(((3-fluoro-6-(1-methyl-1H-pyrazol-4-yl)pyrazolo[1,5-a]pyridin-4-yl)oxy)methyl)-3-azabicyclo[3.1.1]heptane-3-carboxylate (85 mg, 193 μmol) in DCM (2 mL) was added HCl/EtOAc (4 M, 2 mL) and the reaction was stirred at 20° C. for 30 min. DIPEA (0.5 mL) was added dropwise and the reaction mixture was concentrated in vacuo. The crude was purified by prep HPLC (Column: Welch Xtimate C18 150×25 mm×5 pm; Condition: water (10 mM NH₄HCO₃)-MeCN, 13-33% B, Gradient Time (min) 15, flow rate (mL/min) 25.) to give: The first eluting peak (E1), Peak 1 (10 mg, 15% yield) as yellow oil. LCMS m/z=342.1 (M+H)⁺; and the second eluting peak (E2), Peak 2, (30 mg, 47% yield) as yellow oil. LCMS m/z=342.1 (M+H)+

### 5. Example 84: Synthesis of 1-((1R,5S,6s) or (1R,5S,6r)-6-(((3-fluoro-6-(1-methyl-1H-pyrazol-4-yl)pyrazolo[1,5-a]pyridin-4-yl)oxy)methyl)-3-azabicyclo[3.1.1]heptan-3-yl)prop-2-en-1-one

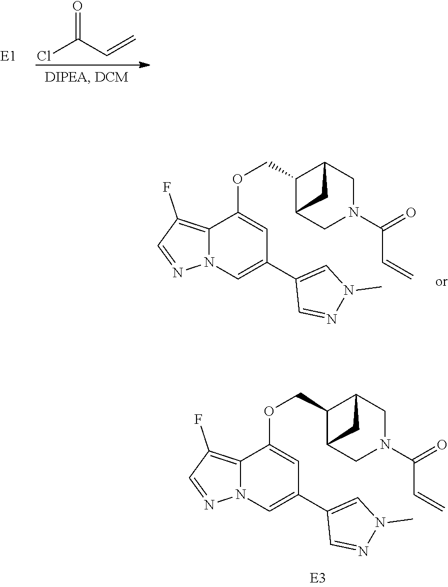

To a mixture of E1, (1R,5S,6s) or (1R,5R,6r)-6-(((3-fluoro-6-(1-methyl-1H-pyrazol-4-yl)pyrazolo[1,5-a]pyridin-4-yl)oxy)methyl)-3-azabicyclo[3.1.1]heptane (10 mg, 29 μmol) and DIPEA (7.6 mg, 59 μmol) in DCM (2 mL) was added acryloyl chloride (5.3 mg, 59 μmol) at 0° C. and the reaction stirred for 10 min. MeOH (0.5 mL) was added dropwise and the reaction mixture was concentrated in vacuo. The crude product was purified by prep HPLC (Column: Welch Xtimate C18 150×25 mm×5 μm; Condition: water (10 mM NH₄HCO₃)-MeCN, 21-41% B, Gradient Time (min) 10, Flow Rate (mL/min) 25.) to give 1-((1R,5S,6s) or (1R,5S,6r)-6-(((3-fluoro-6-(1-methyl-1H-pyrazol-4-yl)pyrazolo[1,5-a]pyridin-4-yl)oxy)methyl)-3-azabicyclo[3.1.1]heptan-3-yl)prop-2-en-1-one (3.0 mg, 26% yield) as yellow oil. LCMS m/z=396.1 (M+H)⁺. 1H NMR (500 MHz, MeOH-d₄) δ=8.23 (s, 1H), 8.06 (s, 1H), 7.90 (s, 1H), 7.76 (d, J=3.5 Hz, 1H), 6.81-6.74 (m, 2H), 6.33-6.28 (m, 1H), 5.76 (dd, J1=2.0 Hz, J2=10.5 Hz, 1H), 4.28-4.16 (m, 2H), 4.01-3.91 (m, 5H), 3.81-3.73 (m, 2H), 2.97-2.85 (m, 1H), 2.72 (t, J=6.0 Hz, 2H), 2.30-2.23 (m, 1H), 1.50 (d, J=9.5 Hz, 1H).

### 6. Example 85: Synthesis of 1-((1R,5S,6r) or (1R,5S,6s)-6-(((3-fluoro-6-(1-methyl-1H-pyrazol-4-yl)pyrazolo[1,5-a]pyridin-4-yl)oxy)methyl)-3-azabicyclo[3.1.1]heptan-3-yl)prop-2-en-1-one

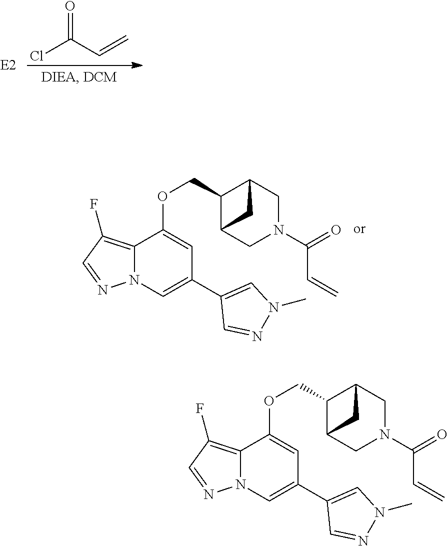

1-((1R,5S,6r) or (1R,5S,6s)-6-(((3-fluoro-6-(1-methyl-1H-pyrazol-4-yl)pyrazolo[1,5-a]pyridin-4-yl)oxy)methyl)-3-azabicyclo[3.1.1]heptan-3-yl)prop-2-en-1-one was obtained as a yellow oil, 10.2 mg, 29% yield from E2, (1R,5S,6r) or (1R,5S,6s)-6-(((3-fluoro-6-(1-methyl-1H-pyrazol-4-yl)pyrazolo[1,5-a]pyridin-4-yl)oxy)methyl)-3-azabicyclo[3.1.1]heptane and acryloyl chloride following the procedure described in step 5. LCMS m/z=396.2 (M+H)⁺. 1H NMR (500 MHz, MeOH-d₄) δ=8.24 (s, 1H), 8.07 (s, 1H), 7.92 (s, 1H), 7.77 (d, J=3.0 Hz, 1H), 6.90-6.72 (m, 2H), 6.35-6.31 (m, 1H), 5.80 (dd, J1=2.0 Hz, J2=10.5 Hz, 1H), 4.52 (d, J=7.5 Hz, 2H), 4.10-3.98 (m, 2H), 3.96 (s, 3H), 3.91-3.85 (m, 1H), 3.83-3.76 (m, 1H), 2.70-2.64 (m, 1H), 2.61-2.55 (m, 2H), 2.38-2.23 (m, 1H), 1.56-1.47 (m, 1H).

### Example 86: 1-(6-((3-fluoro-6-(1-methyl-1H-pyrazol-4-yl)pyrazolo[1,5-a]pyrazin-4-yl)oxy)-6-methyl-2-azaspiro[3.3]heptan-2-yl)but-2-yn-1-one

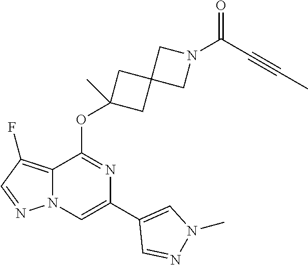 [SMILES: CC#CC(=O)N1CC2(C1)CC(C)(Oc1nc(-c3cnn(C)c3)cn3ncc(F)c13)C2]

### 1. Synthesis of tert-butyl 6-((6-chloro-3-fluoropyrazolo[1,5-a]pyrazin-4-yl)oxy)-6-methyl-2-azaspiro[3.3]heptane-2-carboxylate

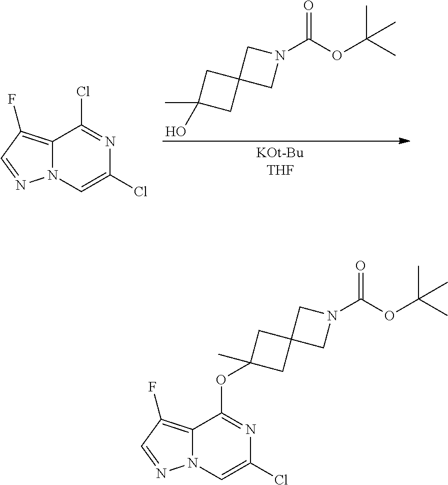 [SMILES: Fc1cnn2cc(Cl)nc(Cl)c12 ; CC1(O)CC2(CN(C(=O)OC(C)(C)C)C2)C1 ; CC(C)(C)OC(=O)N1CC2(C1)CC(C)(Oc1nc(Cl)cn3ncc(F)c13)C2]

KOtBu (1.0 M in THF, 352 μL) was added to a solution of tert-butyl 6-hydroxy-6-methyl-2-azaspiro[3.3]heptane-2-carboxylate (80.0 mg, 352 μmol) in THF (3.0 mL) the mixture stirred for 5 mins, then concentrated to dryness. The tan foam solid was dissolved in THF (3 mL) and 4,6-dichloro-3-fluoro-pyrazolo[1,5-a]pyrazine (72.5 mg, 352 μmol) was added. The mixture was stirred for 15 mins at RT, then for 75 mins at 40° C. The reaction mixture was then concentrated to dryness and purified directly via silica gel chromatography (heptane to EtOAc) to give tert-butyl 6-((6-chloro-3-fluoropyrazolo[1,5-a]pyrazin-4-yl)oxy)-6-methyl-2-azaspiro[3.3]heptane-2-carboxylate as a white solid (100 mg, 72% yield). LCMS: m/z=297.0 (M-CO₂t-Bu+H)⁺.

### 2. Synthesis of tert-butyl 6-((3-fluoro-6-(1-methyl-1H-pyrazol-4-yl)pyrazolo[1,5-a]pyrazin-4-yl)oxy)-6-methyl-2-azaspiro[3.3]heptane-2-carboxylate

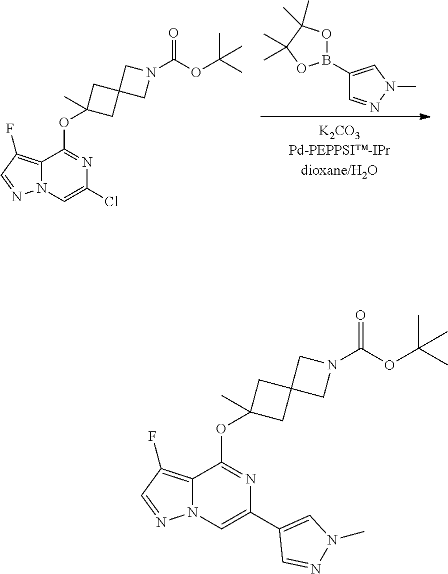

To a vial was added tert-butyl 6-((6-chloro-3-fluoropyrazolo[1,5-a]pyrazin-4-yl)oxy)-6-methyl-2-azaspiro[3.3]heptane-2-carboxylate (225 mg, 567 μmol), 1-methyl-4-(4,4,5,5-tetramethyl-1,3,2-dioxaborolan-2-yl)pyrazole (210 mg, 1.01 mmol), K₂CO₃ (210.2 mg, 1.52 mmol), and Pd-PEPPSI™-IPr (10.0 mg, 14.7 μmol) followed by dioxane (2.0 mL) and water (1.0 mL). The vial was sealed and placed on a preheated 90° C. hot plate and was stirred for 30 mins. The reaction was cooled to RT diluted with water (2 mL) and extracted with EtOAc (3×3 mL). The combined organic layers were concentrated to dryness, then purified via silica gel chromatography (heptane to EtOAc) to give tert-butyl 6-((3-fluoro-6-(1-methyl-1H-pyrazol-4-yl)pyrazolo[1,5-a]pyrazin-4-yl)oxy)-6-methyl-2-azaspiro[3.3]heptane-2-carboxylate as an off-white solid, (250 mg, 100% yield). LC-MS: m/z=465.1 (M+Na)⁺.

### 3. Synthesis of 3-fluoro-6-(1-methyl-1H-pyrazol-4-yl)-4-((6-methyl-2-azaspiro[3.3]heptan-6-yl)oxy)pyrazolo[1,5-a]pyrazine hydrochloride

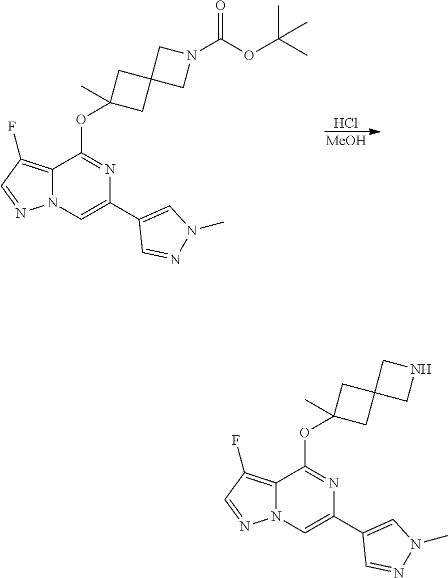 [SMILES: "MeOH" ; Cn1cc(-c2cn3ncc(F)c3c(OC3(C)CC4(CN(C(=O)OC(C)(C)C)C4)C3)n2)cn1 ; Cl ; Cn1cc(-c2cn3ncc(F)c3c(OC3(C)CC4(CNC4)C3)n2)cn1]

To a vial containing tert-butyl 6-((3-fluoro-6-(1-methyl-1H-pyrazol-4-yl)pyrazolo[1,5-a]pyrazin-4-yl)oxy)-6-methyl-2-azaspiro[3.3]heptane-2-carboxylate (250 mg, 565 μmol) was added MeOH (3.0 mL) and HCl (1.25 M in EtOH, 2.9 mL). The vial was then placed on a 30° C. hot plate and stirred overnight. The reaction was concentrated to dryness to give 3-fluoro-6-(1-methyl-1H-pyrazol-4-yl)-4-((6-methyl-2-azaspiro[3.3]heptan-6-yl)oxy)pyrazolo[1,5-a]pyrazine hydrochloride as a white solid. This material was used without further purification. LCMS: m/z=343.1 (M+H)⁺.

### 4. Synthesis of 1-(6-((3-fluoro-6-(1-methyl-1H-pyrazol-4-yl)pyrazolo[1,5-a]pyrazin-4-yl)oxy)-6-methyl-2-azaspiro[3.3]heptan-2-yl)but-2-yn-1-one

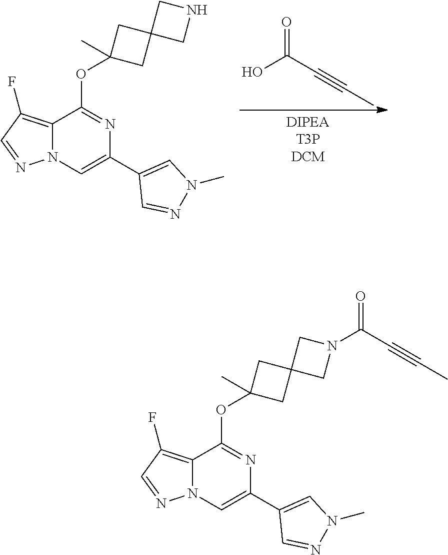

To a vial was added 3-fluoro-6-(1-methyl-1H-pyrazol-4-yl)-4-((6-methyl-2-azaspiro[3.3]heptan-6-yl)oxy)pyrazolo[1,5-a]pyrazine hydrochloride (95 mg, 277 μmol), DCM (5.0 mL), 2-butynoic acid (50 mg, 595 μmol), DIPEA (250.0 μL, 1.44 mmol), and T3P (350 mg, 550 μmol, 50% solution in DMF) and the mixture was stirred at 35° C. for 2 h. The reaction was purified directly via silica gel chromatography (heptane to EtOAc to 3:1 EtOAc:EtOH) to give 1-(6-((3-fluoro-6-(1-methyl-1H-pyrazol-4-yl)pyrazolo[1,5-a]pyrazin-4-yl)oxy)-6-methyl-2-azaspiro[3.3]heptan-2-yl)but-2-yn-1-one (75.0 mg, 65% yield over 2 steps). LCMS: m/z=409.1 (M+H)⁺. ¹H NMR (500 MHz, DMSO-d₆) δ ppm=1.70 (d, J=3.66 Hz, 3H) 1.97-2.02 (m, 3H) 2.65-2.76 (m, 3H) 3.15-3.18 (m, 1H) 3.89 (d, J=1.83 Hz, 4H) 4.05-4.14 (m, 2H) 4.32 (s, 1H) 7.99 (s, 1H) 8.07 (dd, J=3.66, 1.22 Hz, 1H) 8.16 (s, 1H) 8.61 (s, 1H).

### Example 87. N-(5-(6-(1-methyl-1H-pyrazol-4-yl)pyrazolo[1,5-a]pyridin-4-yl)-5-azaspiro[2.4]heptan-7-yl)acrylamide

### 1. Synthesis of N-(5-(6-(1-methyl-1H-pyrazol-4-yl)pyrazolo[1,5-a]pyridin-4-yl)-5-azaspiro 2.4 heptan-7-yl)acrylamide

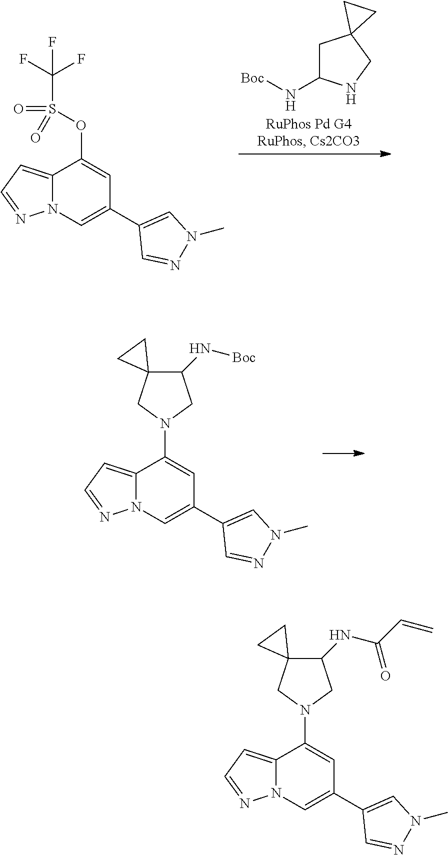 [SMILES: Cn1cc(-c2cc(OS(=O)(=O)C(F)(F)F)c3ccnn3c2)cn1 ; CC(C)(C)OC(=O)NC1CC2(CC2)CN1 ; Cn1cc(-c2cc(N3CC(NC(=O)OC(C)(C)C)C4(CC4)C3)c3ccnn3c2)cn1 ; C=CC(=O)NC1CN(c2cc(-c3cnn(C)c3)cn3nccc23)CC12CC2]

tert-Butyl (5-azaspiro[2.4]heptan-6-yl)carbamate (59 mg, 280 μmol), 6-(1-methyl-1H-pyrazol-4-yl)pyrazolo[1,5-a]pyridin-4-yl trifluoromethanesulfonate (Intermediate C, 97 mg, 280 μmol) and Cs₂CO₃ (274 mg, 840 μmol) were mixed in dry dioxane (0.5 mL). Ruphos Pd G4 (11.9 mg, 10 μmol) (0.035 mL of stock solution in dioxane), and RuPhos (6.5 mg, 10 μmol) (0.035 mL of stock solution in dioxane) were added in one portion in an inert atmosphere. The reaction mixture was sealed and heated with shaking for 16 h at 100° C. The reaction mixture was cooled, filtered and a mixture of TFA (92.5% v/v), water (5% v/v) and TIPS (2.5% v/v) (0.7 mL total) was added in one portion. The reaction mixture was stirred for 6 h at ambient temperature. The reaction mixture was concentrated under reduced pressure and the residue was diluted with dry DCM (0.5 mL). Acryloyl chloride (35.5 mg, 390 μmol) and DIPEA (253 mg, 300 μmol) were added and the reaction was sealed and left at ambient temperature for 16 h. The mixture was evaporated in vacuo and the residue was dissolved in DMSO (0.5 mL) and purified by prep. HPLC (Waters SunFire C18 19*100 5 mkm column; gradient mixture H₂O-MeCN as a mobile phase) to afford N-(5-(6-(1-methyl-1H-pyrazol-4-yl)pyrazolo[1,5-a]pyridin-4-yl)-5-azaspiro[2.4]heptan-7-yl)acrylamide, 3.7 mg, 3.7% yield. LCMS m/z=363.2 (M+H)+

### Example 88. 1-(5-((6-(1-methyl-1H-pyrazol-4-yl)pyrazolo[1,5-a]pyridin-4-yl)amino)-2-azabicyclo[2.2.1]heptan-2-yl)prop-2-en-1-one

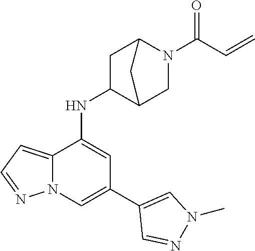 [SMILES: C=CC(=O)N1CC2CC1CC2Nc1cc(-c2cnn(C)c2)cn2nccc12]

1-(5-((6-(1-Methyl-1H-pyrazol-4-yl)pyrazolo[1,5-a]pyridin-4-yl)amino)-2-azabicyclo[2.2.1]heptan-2-yl)prop-2-en-1-one was obtained, 1.6 mg, 2.2% yield from 6-(1-methyl-1H-pyrazol-4-yl)pyrazolo[1,5-a]pyridin-4-yl trifluoromethanesulfonate (Intermediate C) and tert-butyl 5-amino-2-azabicyclo[2.2.1]heptane-2-carboxylate, following the procedure described in Example 87. LCMS m/z=363.2 (M+H)+

### Example 89. N-(5,5-difluoro-1-(6-(1-methyl-1H-pyrazol-4-yl)pyrazolo[1,5-a]pyridin-4-yl)piperidin-3-yl)acrylamide

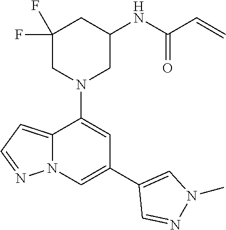 [SMILES: C=CC(=O)NC1CN(c2cc(-c3cnn(C)c3)cn3nccc23)CC(F)(F)C1]

### 1. Synthesis of N-(5,5-difluoro-1-(6-(1-methyl-1H-pyrazol-4-yl)pyrazolo[1,5-a]pyridin-4-yl)piperidin-3-yl)acrylamide

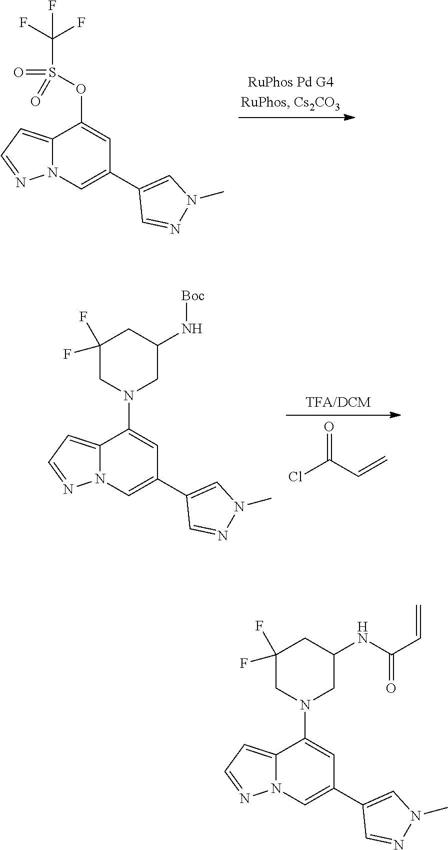

tert-Butyl (5,5-difluoropiperidin-3-yl)carbamate (1.2 equiv.), 6-(1-methyl-1H-pyrazol-4-yl)pyrazolo[1,5-a]pyridin-4-yltrifluoromethanesulfonate (Intermediate C, 1.0 equiv.) and Cs₂CO₃ (3.0 equiv.) were mixed in dry dioxane (1 mL). RuPhos Pd G4 (0.05 equiv.) and RuPhos (0.05 eq.) were added in one portion under an inert atmosphere and the reaction mixture was sealed and heated with shaking for 16 h at 100° C. The reaction mixture was diluted with H₂O (10 mL) and extracted with EtOAc (3×10 mL). The combined organic layers were washed with brine (10 mL), dried over Na₂SO₄, filtered and concentrated in vacuo to give tert-butyl (5,5-difluoro-1-(6-(1-methyl-1H-pyrazol-4-yl)pyrazolo[1,5-a]pyridin-4-yl)piperidin-3-yl)carbamate.

A solution of TFA (5 eq.) in DCM (1 mL) was added to a solution of tert-butyl (5,5-difluoro-1-(6-(1-methyl-1H-pyrazol-4-yl)pyrazolo[1,5-a]pyridin-4-yl)piperidin-3-yl)carbamate (1 equiv.) in DCM (2 mL) and the resulting solution was stirred for 14 h at RT. NaHCO₃ solution was added to the reaction mixture to pH 7-8, the layers separated and the organic phase was concentrated in vacuo to give 5,5-difluoro-1-(6-(1-methyl-1H-pyrazol-4-yl)pyrazolo[1,5-a]pyridin-4-yl)piperidin-3-amine.

To a solution of 5,5-difluoro-1-(6-(1-methyl-1H-pyrazol-4-yl)pyrazolo[1,5-a]pyridin-4-yl)piperidin-3-amine (1 equiv.) in DCM (10 mL) was added DIPEA (1.1 equiv.) the reaction mixture was cooled to 0° C. and acryloyl chloride (1.05 equiv.) was added and the reaction was stirred for 4 h at RT. The reaction mixture was washed with water (10 mL), dried over Na₂SO₄, filtered and concentrated in vacuo. The crude product was purified by prep. HPLC (column: Chromatorex 18 SMB100-5T; 0-1-6 min H₂O/MeOH/0.1% NH₄OH, flow: 30 mL/min; 100×19 mm 5 um) to afford N-(5,5-difluoro-1-(6-(1-methyl-1H-pyrazol-4-yl)pyrazolo[1,5-a]pyridin-4-yl)piperidin-3-yl)acrylamide. LCMS m/z=387.2 (M+H)+

### Example 90. 1-(4-((6-(1-methyl-1H-pyrazol-4-yl)pyrazolo[1,5-a]pyrazin-4-yl)methyl)-1,4-diazepan-1-yl)prop-2-en-1-one

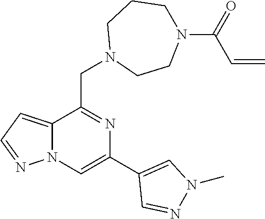 [SMILES: C=CC(=O)N1CCCN(Cc2nc(-c3cnn(C)c3)cn3nccc23)CC1]

### 1. Synthesis of 6-(1-methyl-1H-pyrazol-4-yl)-4-vinylpyrazolo[1,5-a]pyrazine

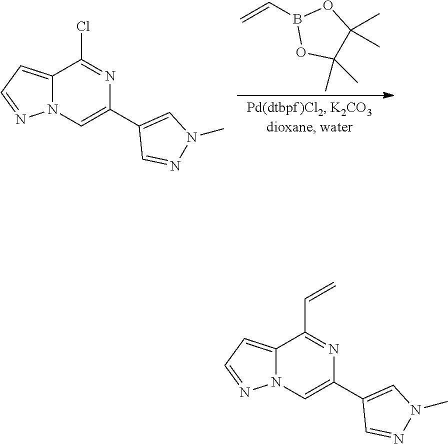 [SMILES: Cn1cc(-c2cn3nccc3c(Cl)n2)cn1 ; C=CB1OC(C)(C)C(C)(C)O1 ; C=Cc1nc(-c2cnn(C)c2)cn2nccc12]

To a solution of 4-chloro-6-(1-methyl-1H-pyrazol-4-yl)pyrazolo[1,5-a]pyrazine (Intermediate A, 0.5 g, 2.14 mmol) and 4,4,5,5-tetramethyl-2-vinyl-1,3,2-dioxaborolane (583 mg, 3.79 mmol) in dioxane (15 mL) and water (1.5 mL) was added K₂CO₃ (887 mg, 6.42 mmol) followed by Pd(dtbpf)Cl₂ (139 mg, 214 μmol) and the reaction was stirred at 90° C. under N₂ for 5 h. The mixture was concentrated in vacuum and the crude product was purified by column chromatography on silica gel eluting with (PE/EtOAc=1/0 to 1/1) to give 6-(1-methyl-1H-pyrazol-4-yl)-4-vinylpyrazolo[1,5-a]pyrazine (180 mg, 36% yield) as a yellow solid. LCMS m/z=226.1 (M+H)+

### 2. Synthesis of 6-(1-methyl-1H-pyrazol-4-yl)pyrazolo[1,5-a]pyrazine-4-carbaldehyde

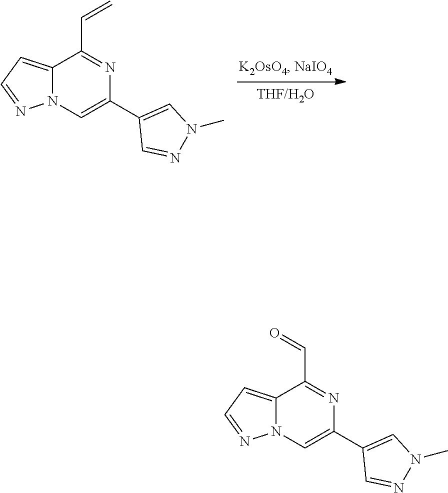 [SMILES: C=Cc1nc(-c2cnn(C)c2)cn2nccc12 ; Cn1cc(-c2cn3nccc3c(C=O)n2)cn1]

To a solution of 6-(1-methyl-1H-pyrazol-4-yl)-4-vinylpyrazolo[1,5-a]pyrazine (0.18 g, 799 μmol) in THF (10 mL) and water (10 mL) was added NaIO₄ (393 mg, 1.84 mmol). K₂OsO₄ (15 mg, 40 μmol) was added and the reaction was stirred at 20° C. for 4 h. The mixture was concentrated in vacuum and the crude was purified by column chromatography on silica gel eluting with (PE/EtOAc=1/0 to 1/1) to give 6-(1-methyl-1H-pyrazol-4-yl)pyrazolo[1,5-a]pyrazine-4-carbaldehyde (60 mg, 26% yield) as a yellow solid. LCMS m/z=228.1 (M+H)+

### 3. Synthesis of tert-butyl 4-((6-(1-methyl-1H-pyrazol-4-yl)pyrazolo[1,5-a]pyrazin-4-yl)methyl)-1,4-diazepane-1-carboxylate

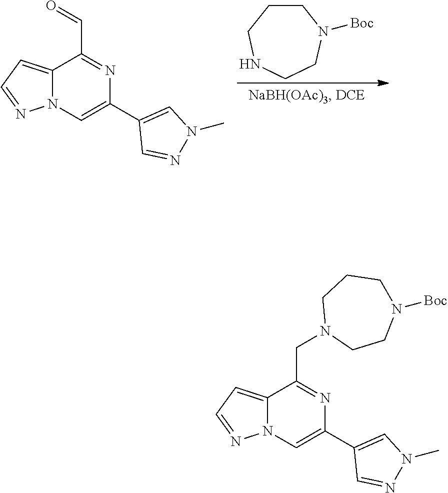 [SMILES: Cn1cc(-c2cn3nccc3c(C=O)n2)cn1 ; CC(C)(C)OC(=O)N1CCCNCC1 ; Cn1cc(-c2cn3nccc3c(CN3CCCN(C(=O)OC(C)(C)C)CC3)n2)cn1]

To a solution of 6-(1-methyl-1H-pyrazol-4-yl)pyrazolo[1,5-a]pyrazine-4-carbaldehyde (126 mg, 555 μmol) and tert-butyl 1,4-diazepane-1-carboxylate (133 mg, 665 μmol) in DCE (30 mL) was added NaBH(OAc)₃ (588 mg, 2.77 mmol) and the mixture was stirred at 20° C. for 4 h. The reaction mixture was concentrated under vacuum and the crude product was purified by silica gel column chromatography (PE/EtOAc=1/4) to give tert-butyl 4-((6-(1-methyl-1H-pyrazol-4-yl)pyrazolo[1,5-a]pyrazin-4-yl)methyl)-1,4-diazepane-1-carboxylate (0.2 g, crude) as a yellow solid. LCMS m/z=412.2 (M+H)+

### 4. Synthesis of 4-((1,4-diazepan-1-yl)methyl)-6-(1-methyl-1H-pyrazol-4-yl)pyrazolo[1,5-a]pyrazine hydrochloride

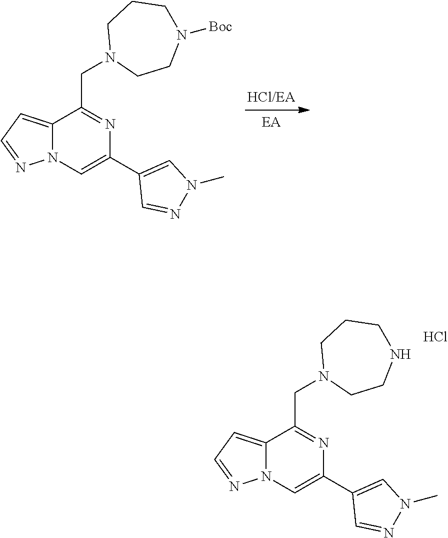 [SMILES: Cn1cc(-c2cn3nccc3c(CN3CCCN(C(=O)OC(C)(C)C)CC3)n2)cn1 ; Cn1cc(-c2cn3nccc3c(CN3CCCNCC3)n2)cn1 ; Cl]

To a solution of tert-butyl 4-((6-(1-methyl-1H-pyrazol-4-yl)pyrazolo[1,5-a]pyrazin-4-yl)methyl)-1,4-diazepane-1-carboxylate (180 mg, 437 μmol) in EtOAc (10 mL) was added HCl/EtOAc (4 M, 10 mL) and the mixture was stirred at 20° C. for 30 min. The mixture was concentrated under vacuum to give 4-((1,4-diazepan-1-yl)methyl)-6-(1-methyl-1H-pyrazol-4-yl)pyrazolo[1,5-a]pyrazine hydrochloride (0.13 g, crude) as a yellow solid that was used for the next step without purification. LCMS m/z=312.2 (M+H)+

### 5. Synthesis of 1-(4-((6-(1-methyl-1H-pyrazol-4-yl)pyrazolo[1,5-a]pyrazin-4-yl)methyl)-1,4-diazepan-1-yl)prop-2-en-1-one

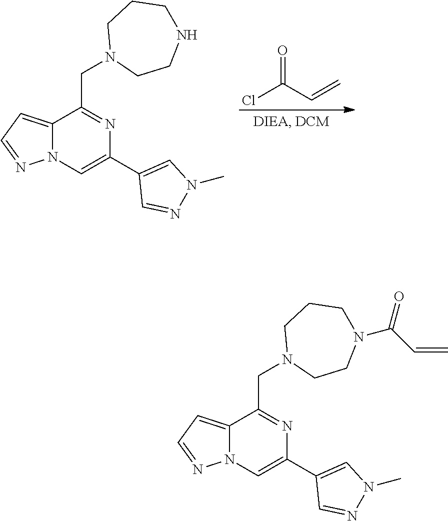

To a solution of 4-((1,4-diazepan-1-yl)methyl)-6-(1-methyl-1H-pyrazol-4-yl)pyrazolo[1,5-a]pyrazine hydrochloride (50 mg, 161 μmol) in DCM (20 mL) was added DIPEA (62 mg, 482 μmol) then acryloyl chloride (17 mg, 193 μmol) and the mixture was stirred at 20° C. for 30 min. The reaction mixture was concentrated under vacuum and the crude product was purified by prep-HPLC (Column: Welch Xtimate C18 150×25 mm×5 um; Condition: water (10 mM NH₄HCO₃)-MeCN, 22-46% B, Gradient Time (min) 10, Flow Rate (mL/min) 25.) to give 1-(4-((6-(1-methyl-1H-pyrazol-4-yl)pyrazolo[1,5-a]pyrazin-4-yl)methyl)-1,4-diazepan-1-yl)prop-2-en-1-one (49 mg, 83% yield) as a brown solid. LCMS m/z=366.2 (M+H)⁺. 1HNMR (400 MHz, DMSO-d₆) δ: 9.02 (s, 1H), 8.20 (d, J=5.6 Hz, 1H), 8.05 (t, J=2.4 Hz, 1H), 8.02 (s, 1H), 7.07 (t, J=2.4 Hz, 1H), 6.78-6.70 (m, 1H), 6.15-6.09 (m, 1H), 5.68-5.62 (m, 1H), 4.03 (s, 2H), 3.88 (s, 3H), 3.63-3.54 (m, 4H), 2.82-2.78 (m, 2H), 2.69-2.67 (m, 2H), 1.77-1.72 (m, 2H).

### Example 91. 1-(3-(methyl((6-(1-methyl-1H-pyrazol-4-yl)pyrazolo[1,5-a]pyrazin-4-yl)methyl)amino)piperidin-1-yl)prop-2-en-1-one

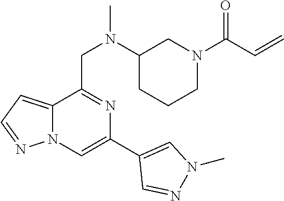 [SMILES: C=CC(=O)N1CCCC(N(C)Cc2nc(-c3cnn(C)c3)cn3nccc23)C1]

### 1. Synthesis of tert-butyl 3-(methyl((6-(1-methyl-1H-pyrazol-4-yl)pyrazolo[1,5-a]pyrazin-4-yl)methyl)amino)piperidine-1-carboxylate

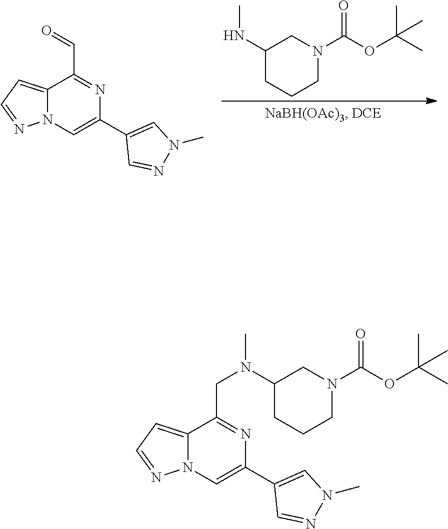 [SMILES: Cn1cc(-c2cn3nccc3c(C=O)n2)cn1 ; CNC1CCCN(C(=O)OC(C)(C)C)C1 ; CN(Cc1nc(-c2cnn(C)c2)cn2nccc12)C1CCCN(C(=O)OC(C)(C)C)C1]

To a solution of 6-(1-methyl-1H-pyrazol-4-yl)pyrazolo[1,5-a]pyrazine-4-carbaldehyde (Example 90, step 2, 100 mg, 440 μmol) and tert-butyl 3-(methylamino)piperidine-1-carboxylate (94 mg, 440 μmol) in DCE (10 mL) was added NaBH(OAc)₃ (466 mg, 2.20 mmol) and the mixture was stirred at 20° C. for 30 min. The reaction mixture was concentrated under vacuum and the crude product was purified by prep TLC (PE/EtOAc=0/1) to give tert-butyl 3-(methyl((6-(1-methyl-1H-pyrazol-4-yl)pyrazolo[1,5-a]pyrazin-4-yl)methyl)amino)piperidine-1-carboxylate (100 mg, 53%) as a yellow oil. LCMS m/z=426.5 (M+H)+

### 2. Synthesis of N-methyl-N-((6-(1-methyl-1H-pyrazol-4-yl)pyrazolo[1,5-a]pyrazin-4-yl)methyl)piperidin-3-amine hydrochloride

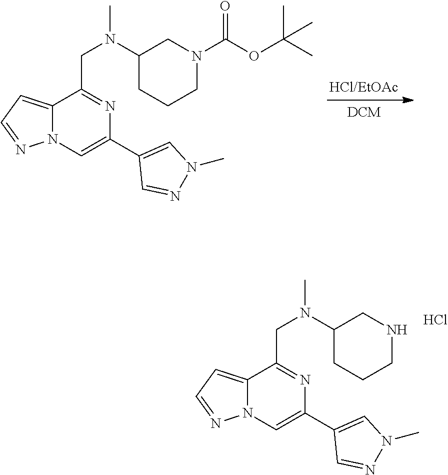 [SMILES: CN(Cc1nc(-c2cnn(C)c2)cn2nccc12)C1CCCN(C(=O)OC(C)(C)C)C1 ; CN(Cc1nc(-c2cnn(C)c2)cn2nccc12)C1CCCNC1 ; Cl]

To a solution of tert-butyl 3-(methyl((6-(1-methyl-1H-pyrazol-4-yl)pyrazolo[1,5-a]pyrazin-4-yl)methyl)amino)piperidine-1-carboxylate (100 mg, 235 μmol) in DCM (10 mL) was added HCl/EtOAc (4 M, 10 mL) and the mixture was stirred at 25° C. for 1 h. The mixture was concentrated under vacuum to give N-methyl-N-((6-(1-methyl-1H-pyrazol-4-yl)pyrazolo[1,5-a]pyrazin-4-yl)methyl)piperidin-3-amine hydrochloride (90 mg, crude) as a yellow solid. LCMS m/z=326.0 (M+H)+

### 3. Synthesis of 1-(3-(methyl((6-(1-methyl-1H-pyrazol-4-yl)pyrazolo[1,5-a]pyrazin-4-yl)methyl)amino)piperidin-1-yl)prop-2-en-1-one

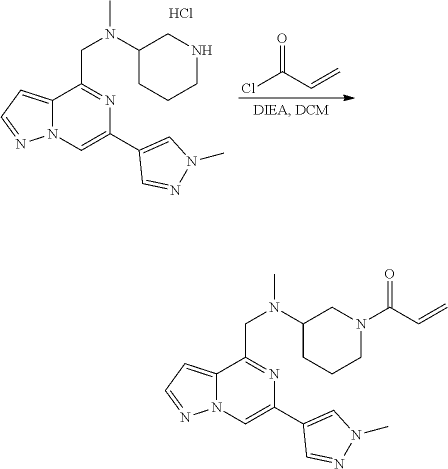

To a solution of N-methyl-N-((6-(1-methyl-1H-pyrazol-4-yl)pyrazolo[1,5-a]pyrazin-4-yl)methyl)piperidin-3-amine hydrochloride (80 mg, 221 μmol) in DCM (20 mL) was added DIPEA (57 mg, 442 μmol) then acryloyl chloride (20 mg, 221 μmol) and the mixture was stirred at 25° C. for 10 min. The mixture was quenched with MeOH (1 mL) and concentrated under vacuum. The residue was purified by prep-HPLC (Column: Welch Xtimate C18 150×25 mm×5 μm; Condition: water (10 mM NH₄HCO₃)-MeCN, 21-51% B, Gradient Time (min) 10, Flow Rate (mL/min): 25) to give 1-(3-(methyl((6-(1-methyl-1H-pyrazol-4-yl)pyrazolo[1,5-a]pyrazin-4-yl)methyl)amino)piperidin-1-yl)prop-2-en-1-one (43 mg, 51% yield) as a yellow solid. LCMS m/z=380.2 (M+H)⁺. 1H NMR (500 MHz, DMSO-d₆) δ ppm=9.02 (s, 1H), 8.22 (d, J=12.5 Hz, 1H), 8.10-7.94 (m, 2H), 7.06 (d, J=11.0 Hz, 1H), 6.85-6.69 (m, 1H), 6.11-5.97 (m, 1H), 5.70-5.54 (m, 1H), 4.56-4.16 (m, 1H), 4.09-4.02 (m, 2H), 3.89 (s, 3H), 3.27-2.96 (m, 2H), 2.88-2.56 (m, 2H), 2.27 (d, J=6.0 Hz, 3H), 2.00-1.74 (m, 2H), 1.69-1.31 (m, 2H).

### Example 92. N-((1S,3R)-3-((3-fluoro-6-(1-methyl-H-pyrazol-4-yl)pyrazolo[1,5-a]pyrazin-4-yl)oxy)cyclohexyl)-N-methylbut-2-ynamide

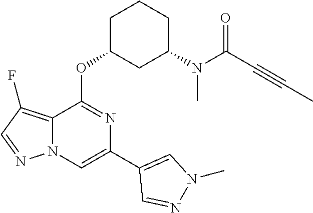 [SMILES: CC#CC(=O)N(C)[C@H]1CCC[C@@H](Oc2nc(-c3cnn(C)c3)cn3ncc(F)c23)C1]

### 1. Synthesis of 4,6-dichloro-3-fluoropyrazolo[1,5-a]pyrazine

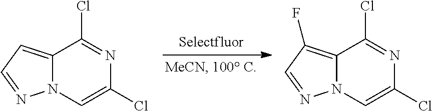 [SMILES: Clc1cn2nccc2c(Cl)n1 ; Fc1cnn2cc(Cl)nc(Cl)c12]

To a solution of Selectfluor (226 g, 638 mmol) in MeCN (1200 mL) and AcOH (120 mL) was added 4,6-dichloropyrazolo[1,5-a]pyrazine (80 g, 426 mmol) and the reaction mixture was stirred at 100° C. for 24 h. The reaction mixture was concentrated under vacuum, water (300 mL) was added and the mixture was extracted with DCM (300 mL×2). The combined organic layers were washed with brine (300 mL), dried over Na₂SO₄, filtered and concentrated. The residue was purified by column chromatography (from 0% to 50%, EtOAc in PE) and the product re-purified by prep. HPLC (Column:Phenomenex Luna C18 (250*80 mm*15 um), Condition: water(0.05% NH₃H₂O+10 mM NH₄HCO₃)-MeCN 40-68% B; Gradient Time (min): 21; FlowRate (mL/min): 250) followed by lyophilization to give 4,6-dichloro-3-fluoropyrazolo[1,5-a]pyrazine (13.5 g, 15% yield) as off-white solid. LCMS m/z=206.0 (M+H)+

### 2. Synthesis of tert-butyl ((1S,3R)-3-((6-chloro-3-fluoropyrazolo[1,5-a]pyrazin-4-yl)oxy)cyclohexyl)carbamate

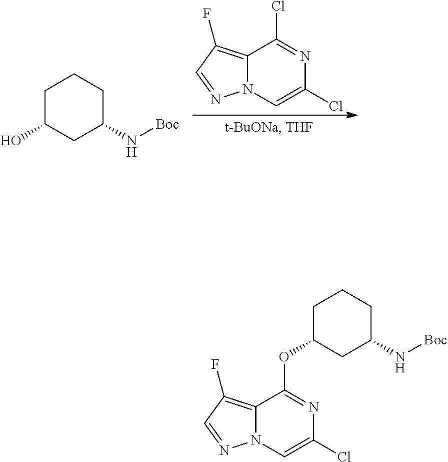 [SMILES: CC(C)(C)OC(=O)N[C@H]1CCC[C@@H](O)C1 ; Fc1cnn2cc(Cl)nc(Cl)c12 ; CC(C)(C)OC(=O)N[C@H]1CCC[C@@H](Oc2nc(Cl)cn3ncc(F)c23)C1]

To a solution of tert-butyl ((1S,3R)-3-hydroxycyclohexyl)carbamate (110 mg, 511 μmol) in THF (2 mL) was added t-BuONa (98 mg, 1.02 mmol) and the mixture was stirred at 0° C. for 10 mins. 4,6-Dichloro-3-fluoropyrazolo[1,5-a]pyrazine (158 mg, 766 μmol) was added and the reaction stirred at 0° C. for 30 mins. The mixture was concentrated under vacuum and the crude product was purified by prep-TLC (PE/EtOAc=4/1) to give tert-butyl ((1S,3R)-3-((6-chloro-3-fluoropyrazolo[1,5-a]pyrazin-4-yl)oxy)cyclohexyl)carbamate (90 mg, 46% yield) as a white solid. LCMS m/z=407.2 (M+H)+

### 3. Synthesis of tert-butyl ((1S,3R)-3-((3-fluoro-6-(1-methyl-1H-pyrazol-4-yl)pyrazolo[1,5-a]pyrazin-4-yl)oxy)cyclohexyl)carbamate

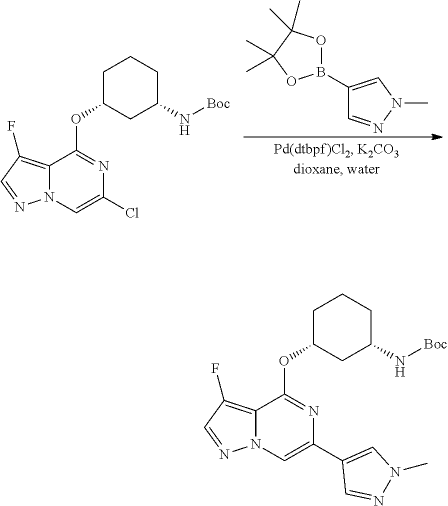

To a solution of tert-butyl ((1S,3R)-3-((6-chloro-3-fluoropyrazolo[1,5-a]pyrazin-4-yl)oxy)cyclohexyl)carbamate (90 mg, 234 μmol) in dioxane (2 mL) and water (0.4 mL) was added 1-methyl-4-(4,4,5,5-tetramethyl-1,3,2-dioxaborolan-2-yl)-1H-pyrazole (73 mg, 351 μmol), K₂CO₃ (97 mg, 702 μmol) and Pd(dtbpf)Cl₂ (15 mg, 230 μmol) and the reaction stirred at 90° C. under N₂ for 2 h. The mixture was concentrated under vacuum and the crude product was purified by prep-TLC (PE/EtOAc=0/1) to give tert-butyl ((1S,3R)-3-((3-fluoro-6-(1-methyl-1H-pyrazol-4-yl)pyrazolo[1,5-a]pyrazin-4-yl)oxy)cyclohexyl)carbamate (80 mg, 79% yield) as a white solid. LCMS m/z=431.2 (M+H)+

### 4. Synthesis of tert-butyl ((1S,3R)-3-((3-fluoro-6-(1-methyl-1H-pyrazol-4-yl)pyrazolo[1,5-a]pyrazin-4-yl)oxy)cyclohexyl)(methyl)carbamate

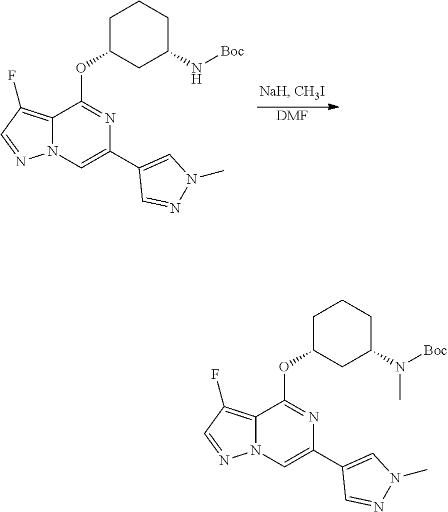

To a solution of tert-butyl ((1S,3R)-3-((3-fluoro-6-(1-methyl-1H-pyrazol-4-yl)pyrazolo[1,5-a]pyrazin-4-yl)oxy)cyclohexyl)carbamate (80 mg, 186 μmol) in DMF (4 mL) was added NaH (22 mg, 558 μmol) and the mixture stirred at 0° C. for 10 mins. Mel (53 mg, 372 μmol) was added and the reaction stirred at 15° C. for 3 h. The mixture was quenched with water (1 mL) and concentrated under vacuum to give tert-butyl ((1S,3R)-3-((3-fluoro-6-(1-methyl-1H-pyrazol-4-yl)pyrazolo[1,5-a]pyrazin-4-yl)oxy)cyclohexyl)(methyl)carbamate (70 mg, crude) as a white solid. LCMS m/z=445.3 (M+H)+

### 5. Synthesis of (1S,3R)-3-((3-fluoro-6-(1-methyl-1H-pyrazol-4-yl)pyrazolo[1,5-a]pyrazin-4-yl)oxy)-N-methylcyclohexan-1-amine

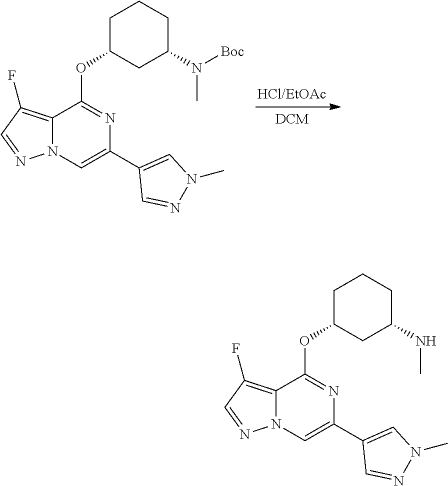 [SMILES: CN(C(=O)OC(C)(C)C)[C@H]1CCC[C@@H](Oc2nc(-c3cnn(C)c3)cn3ncc(F)c23)C1 ; CN[C@H]1CCC[C@@H](Oc2nc(-c3cnn(C)c3)cn3ncc(F)c23)C1]

To a solution of tert-butyl ((1S,3R)-3-((3-fluoro-6-(1-methyl-1H-pyrazol-4-yl)pyrazolo[1,5-a]pyrazin-4-yl)oxy)cyclohexyl)(methyl)carbamate (70 mg, 157 μmol) in DCM (8 mL) was added HCl/EtOAc (5 mL, 4 M) and the reaction stirred at 15° C. for 30 mins. The mixture was concentrated under vacuum and the residue was purified by prep-HPLC (Column: Welch Xtimate C18 150×25 mm×5 pm; Condition: water (10 mM NH₄HCO₃)-MeCN, 19-49% B, Gradient Time (min) 10, Flow Rate (mL/min): 25) to give (1S,3R)-3-((3-fluoro-6-(1-methyl-1H-pyrazol-4-yl)pyrazolo[1,5-a]pyrazin-4-yl)oxy)-N-methylcyclohexan-1-amine (50 mg, 92% yield) as a white solid. LCMS m/z=345.1 (M+H)+

### 6. Synthesis of N-((1S,3R)-3-((3-fluoro-6-(1-methyl-1H-pyrazol-4-yl)pyrazolo[1,5-a]pyrazin-4-yl)oxy)cyclohexyl)-N-methylbut-2-ynamide

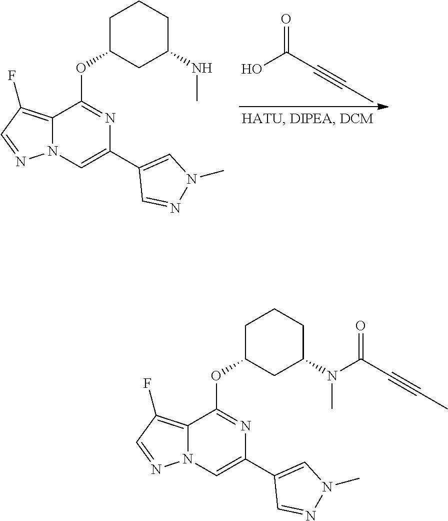

To a solution of (1S,3R)-3-((3-fluoro-6-(1-methyl-1H-pyrazol-4-yl)pyrazolo[1,5-a]pyrazin-4-yl)oxy)-N-methylcyclohexan-1-amine (45 mg, 131 μmol) in DCM (30 mL) was added DIPEA (34 mg, 261 μmol), but-2-ynoic acid (12 mg, 144 μmol) and HATU (50 mg, 131 μmol) and the reaction stirred at 15° C. for 30 mins. The mixture was concentrated under vacuum and the residue was purified by prep-HPLC (Column: Waters Xbridge BEH C18 100×25 mm×5 μm; Condition: water (0.225% FA)-MeCN, Begin B 24 End B 54, Gradient Time (min) 12, Flow Rate (m/min): 25) to give N-((1S,3R)-3-((3-fluoro-6-(1-methyl-1H-pyrazol-4-yl)pyrazolo[1,5-a]pyrazin-4-yl)oxy)cyclohexyl)-N-methylbut-2-ynamide (20 mg, 37% yield) as a yellow solid. LCMS m/z=411.3 (M+H)+. 1H NMR (500 MHz, MeOH-d₄) δ ppm: 8.27 (d, J=4.5 Hz, 1H), 8.08 (d, J=9.5 Hz, 1H), 7.94 (d, J=7.5 Hz, 1H), 7.79-7.84 (m, 1H), 5.45-5.37 (m, 1H), 4.58-4.50 (m, 1H), 3.95 (d, J=1.5 Hz, 3H), 2.89 (s, 3H), 2.42-2.28 (m, 2H), 2.12-2.03 (m, 3H), 1.94-1.50 (m, 6H).

### Example 93. (E)-4-(dimethylamino)-N-((1S,3R)-3-((3-fluoro-6-(1-methyl-1H-pyrazol-4-yl)pyrazolo[1,5-a]pyrazin-4-yl)oxy)cyclohexyl)-N-methylbut-2-enamide

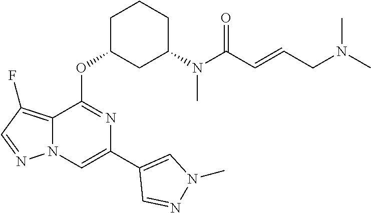 [SMILES: CN(C)C/C=C/C(=O)N(C)[C@H]1CCC[C@@H](Oc2nc(-c3cnn(C)c3)cn3ncc(F)c23)C1]

(E)-4-(Dimethylamino)-N-((1S,3R)-3-((3-fluoro-6-(1-methyl-1H-pyrazol-4-yl)pyrazolo[1,5-a]pyrazin-4-yl)oxy)cyclohexyl)-N-methylbut-2-enamide was obtained as a white solid, from (1S,3R)-3-((3-fluoro-6-(1-methyl-1H-pyrazol-4-yl)pyrazolo[1,5-a]pyrazin-4-yl)oxy)-N-methylcyclohexan-1-amine (Example 92, step 5) and (E)-4-(dimethylamino)but-2-enoic acid, following a similar procedure to that described in Example 92). LCMS m/z=456.2 (M+H)+. 1H NMR (400 MHz, DMSO-d₆) δ ppm: 8.61 (s, 1H), 8.27-8.20 (m, 1H), 8.15-8.07 (m, 1H), 8.01 (s, 1H), 6.77-6.50 (m, 2H), 5.54-5.29 (m, 1H), 4.58-4.10 (m, 1H), 3.89 (s, 3H), 3.13-3.07 (m, 2H), 2.94-2.79 (m, 3H), 2.24-2.15 (m, 8H), 1.88-1.44 (m, 6H).

### Example 94. (E)-4-(dimethylamino)-N-((1S,3R)-3-((3-fluoro-6-(1-methyl-1H-pyrazol-4-yl)pyrazolo[1,5-a]pyrazin-4-yl)oxy)cyclohexyl)but-2-enamide

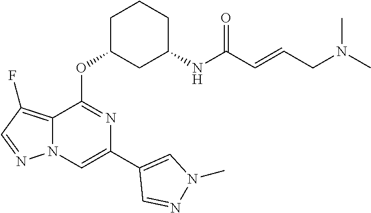 [SMILES: CN(C)C/C=C/C(=O)N[C@H]1CCC[C@@H](Oc2nc(-c3cnn(C)c3)cn3ncc(F)c23)C1]

### 1. Synthesis of (1S,3R)-3-((3-fluoro-6-(1-methyl-1H-pyrazol-4-yl)pyrazolo[1,5-a]pyrazin-4-yl)oxy)cyclohexan-1-amine hydrochloride

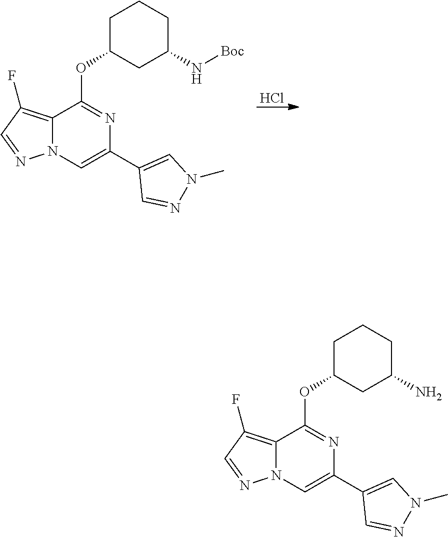 [SMILES: Cn1cc(-c2cn3ncc(F)c3c(O[C@@H]3CCC[C@H](NC(=O)OC(C)(C)C)C3)n2)cn1 ; Cl ; Cn1cc(-c2cn3ncc(F)c3c(O[C@@H]3CCC[C@H](N)C3)n2)cn1]

(1S,3R)-3-((3-Fluoro-6-(1-methyl-1H-pyrazol-4-yl)pyrazolo[1,5-a]pyrazin-4-yl)oxy)cyclohexan-1-amine hydrochloride was prepared from tert-butyl ((1S,3R)-3-((3-fluoro-6-(1-methyl-1H-pyrazol-4-yl)pyrazolo[1,5-a]pyrazin-4-yl)oxy)cyclohexyl)carbamate (step 3, Example 92), following the procedure described in Step 5, Example 92.

### 2. Synthesis of (E)-4-(dimethylamino)-N-((1S,3R)-3-((3-fluoro-6-(1-methyl-1H-pyrazol-4-yl)pyrazolo[1,5-a]pyrazin-4-yl)oxy)cyclohexyl)but-2-enamide

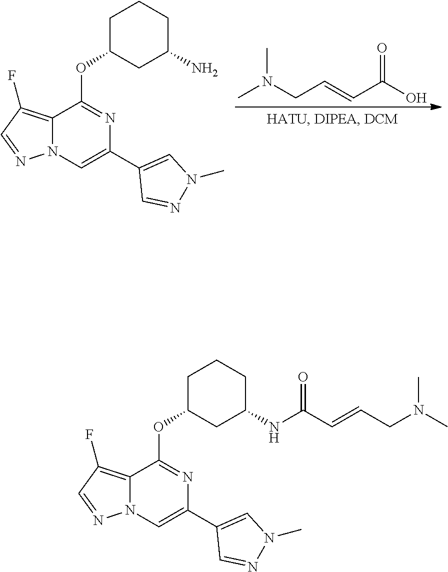

(E)-4-(Dimethylamino)-N-((1S,3R)-3-((3-fluoro-6-(1-methyl-1H-pyrazol-4-yl)pyrazolo[1,5-a]pyrazin-4-yl)oxy)cyclohexyl)but-2-enamide was obtained as a white solid, from (1S,3R)-3-((3-fluoro-6-(1-methyl-1H-pyrazol-4-yl)pyrazolo[1,5-a]pyrazin-4-yl)oxy)cyclohexan-1-amine and (E)-4-(dimethylamino)but-2-enoic acid following a similar method to that described in Example 92. LCMS m/z=442.2 (M+H)+. 1H NMR (400 MHz, DMSO-d₆) δ ppm: 8.58 (d, J=1.2 Hz, 1H), 8.30-8.12 (m, 2H), 8.06-7.98 (m, 2H), 6.5-6.50 (m, 1H), 5.99 (d, J=15.6 Hz, 1H), 5.30 (d, J=10.8 Hz, 1H), 3.88 (s, 4H), 2.95 (d, J=5.6 Hz, 2H), 2.23-2.01 (m, 7H), 1.81 (d, J=11.6 Hz, 2H), 1.52-1.35 (m, 3H), 1.24-1.11 (m, 2H).

### Example 95. N-((1S,3R)-3-((3-fluoro-6-(1-methyl-1H-pyrazol-4-yl)pyrazolo[1,5-a]pyrazin-4-yl)oxy)cyclopentyl) but-2-ynamide

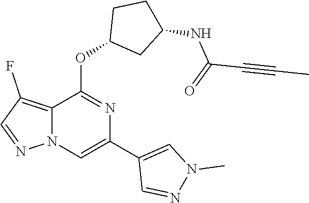 [SMILES: CC#CC(=O)N[C@H]1CC[C@@H](Oc2nc(-c3cnn(C)c3)cn3ncc(F)c23)C1]

1. Synthesis of tert-butyl ((1S,3R)-3-((6-chloro-3-fluoropyrazolo[1,5-a]pyrazin-4-yl)oxy)cyclopentyl)carbamate

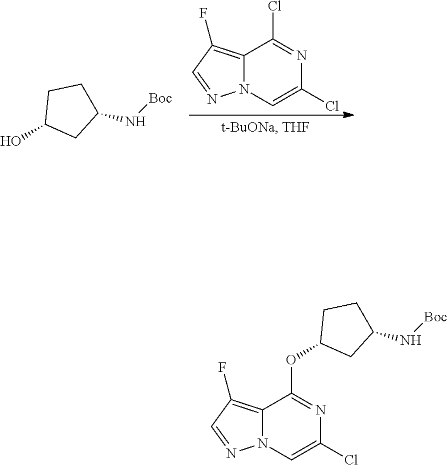 [SMILES: CC(C)(C)OC(=O)N[C@H]1CC[C@@H](O)C1 ; Fc1cnn2cc(Cl)nc(Cl)c12 ; CC(C)(C)OC(=O)N[C@H]1CC[C@@H](Oc2nc(Cl)cn3ncc(F)c23)C1]

tert-Butyl ((1S,3R)-3-((6-chloro-3-fluoropyrazolo[1,5-a]pyrazin-4-yl)oxy)cyclopentyl)carbamate was obtained as a white solid, 140 mg, 76% yield from tert-butyl ((1S,3R)-3-hydroxycyclopentyl)carbamate and dichloro-3-fluoropyrazolo[1,5-a]pyrazine (step 1, Example 92), following the procedure described for step 2, Example 92. LCMS m/z=371.2 (M+H)+

### 2. Synthesis of tert-butyl ((1S,3R)-3-((3-fluoro-6-(1-methyl-1H-pyrazol-4-yl)pyrazolo[1,5-a]pyrazin-4-yl)oxy)cyclopentyl)carbamate

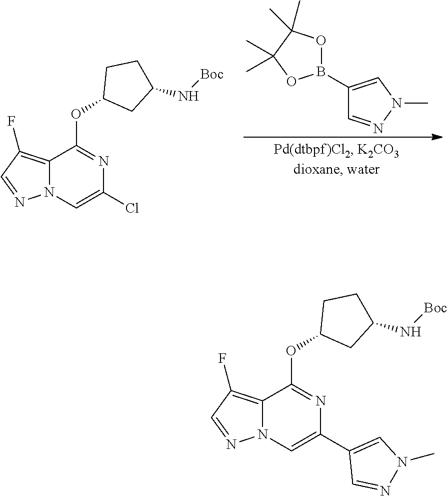

tert-Butyl ((1S,3R)-3-((3-fluoro-6-(1-methyl-1H-pyrazol-4-yl)pyrazolo[1,5-a]pyrazin-4-yl)oxy)cyclopentyl)carbamate was obtained as a white solid, 120 mg, 89% yield, from tert-butyl ((1S,3R)-3-((6-chloro-3-fluoropyrazolo[1,5-a]pyrazin-4-yl)oxy)cyclopentyl)carbamate, following the procedure described for step 3, Example 92. LCMS m/z=417.2 (M+H)+

### 3. Synthesis of (1S,3R)-3-((3-fluoro-6-(1-methyl-1H-pyrazol-4-yl)pyrazolo[1,5-a]pyrazin-4-yl)oxy)cyclopentan-1-amine hydrochloride

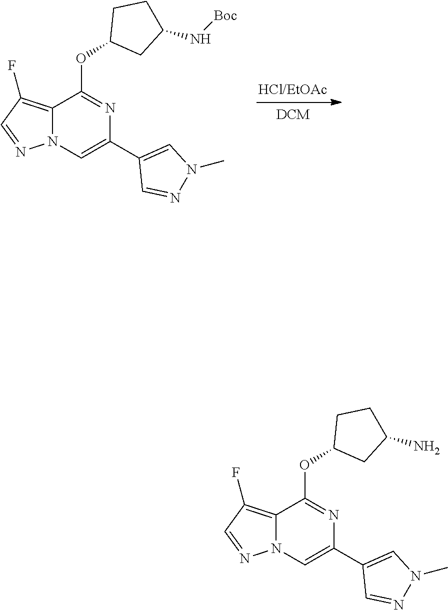 [SMILES: Cn1cc(-c2cn3ncc(F)c3c(O[C@@H]3CC[C@H](NC(=O)OC(C)(C)C)C3)n2)cn1 ; Cn1cc(-c2cn3ncc(F)c3c(O[C@@H]3CC[C@H](N)C3)n2)cn1]

A mixture of tert-butyl ((1S,3R)-3-((3-fluoro-6-(1-methyl-1H-pyrazol-4-yl)pyrazolo[1,5-a]pyrazin-4-yl)oxy)cyclopentyl)carbamate (500 mg, 1.20 mmol) in HCl/EtOAc (4 M, 7.14 mL) was stirred at 15° C. for 1 h. The reaction mixture was evaporated under reduced pressure to give (1S,3R)-3-((3-fluoro-6-(1-methyl-1H-pyrazol-4-yl)pyrazolo[1,5-a]pyrazin-4-yl)oxy)cyclopentan-1-amine hydrochloride (400 mg, crude) as a yellow solid. LCMS m/z=317.1 (M+H)+

### 4. Synthesis of N-((1S,3R)-3-((3-fluoro-6-(1-methyl-1H-pyrazol-4-yl)pyrazolo[1,5-a]pyrazin-4-yl)oxy)cyclopentyl)-N-methylbut-2-ynamide

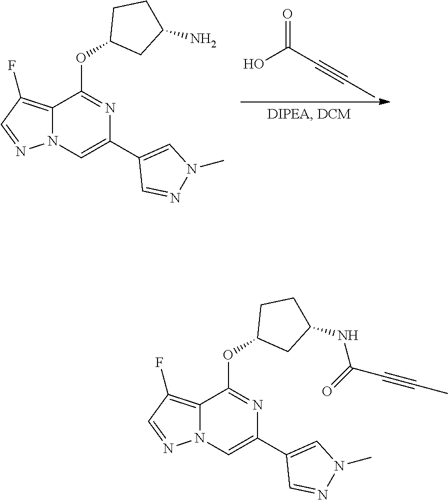

DIPEA (98 mg, 759 μmol) and but-2-ynoic acid (58 mg, 685 μmol) were added to a solution of (1S,3R)-3-((3-fluoro-6-(1-methyl-1H-pyrazol-4-yl)pyrazolo[1,5-a]pyrazin-4-yl)oxy)cyclopentan-1-amine hydrochloride (120 mg, 379 μmol) in DCM (8 mL) and the mixture stirred for 30 min. HATU (174 mg, 455 μmol) was added and the reaction mixture was stirred at 20° C. for 1 h. The solvent was removed in vacuo and the crude was purified by prep HPLC (Column: Welch Xtimate C18 150×25 mm×5 μm, Condition: water (10 mM NH₄HCO₃)-MeCN, Begin B 28, End B 58, Gradient Time (min) 10, Flow Rate (mL/min) 25) to give N-((1S,3R)-3-((3-fluoro-6-(1-methyl-1H-pyrazol-4-yl)pyrazolo[1,5-a]pyrazin-4-yl)oxy)cyclopentyl)-N-methylbut-2-ynamide (62 mg, 43% yield.) as a white solid. LCMS m/z=383.1 (M+H)+. 1H NMR (500 MHz, MeOH-d₄) δ=8.29 (d, J=1.5 Hz, 1H), 8.10 (s, 1H), 7.95 (s, 1H), 7.84 (d, J=3.5 Hz, 1H), 5.72-5.67 (m, 1H), 4.33-4.25 (m, 1H), 3.96 (s, 3H), 2.69-2.65 (m, 1H), 2.19-2.09 (m, 3H), 1.97 (s, 3H), 1.91-1.80 (m, 2H).

### Example 96. N-methyl-N-((cis)-5-((6-(1-methyl-1H-pyrazol-4-yl)pyrazolo[1,5-a]pyrazin-4-yl)oxy)tetrahydro-2H-pyran-3-yl)acrylamide

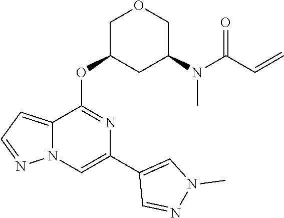 [SMILES: C=CC(=O)N(C)[C@@H]1COC[C@H](Oc2nc(-c3cnn(C)c3)cn3nccc23)C1]

### 1. Synthesis of 5-ethoxy-2H-pyran-3(6H)-one

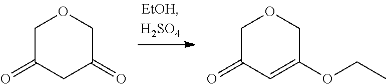

To a solution 2H-pyran-3,5(4H,6H)-dione (5 g, 43.8 mmol) in EtOH (100 mL) was added H₂SO₄ (5.37 mL, 101 mmol) and the reaction was stirred at 25° C. for 12 h. The mixture was quenched with saturated NaHCO₃ aqueous solution (50 mL) then concentrated under reduced pressure to remove EtOH. The residue was extracted with EtOAc (60 mL×3), the combined organic layer was washed with brine (80 mL), dried over Na₂SO₄, filtered and the filtrate was concentrated in vacuo. The crude product was purified by CombiFlash® (PE/EtOAc=5/1) to give 5-ethoxy-2H-pyran-3(6H)-one (2.4 g, 39% yield) as a white solid. LCMS m/z=143.3 (M+H)+

### 2. Synthesis of 5-amino-2H-pyran-3(6H)-one

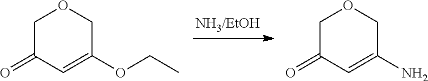

A solution of 5-ethoxy-2H-pyran-3(6H)-one (2.4 g, 16.9 mmol) in EtOH (50 mL) was cooled in a dry ice bath, NH₃ (g) was bubbled through with stirring for 10 min and the reaction mixture was stirred at 25° C. for 12 h. The mixture was concentrated under vacuum and the crude product was purified by CombiFlash® (PE/EtOAc=0/1) to give 5-amino-2H-pyran-3(6H)-one (1.78 g, 93% yield) as a white solid. 1H NMR (400 MHz, DMSO-d₆) δ=7.41-6.75 (m, 2H), 5.01 (s, 1H), 4.18 (s, 2H), 3.80 (s, 2H).

### 3. Synthesis of tert-butyl (5-oxo-5,6-dihydro-2H-pyran-3-yl)carbamate

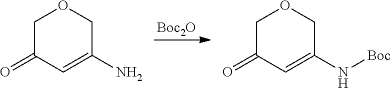 [SMILES: "Boc2O" ; NC1=CC(=O)COC1 ; CC(C)(C)OC(=O)NC1=CC(=O)COC1]

To a solution of 5-amino-2H-pyran-3(6H)-one (1.78 g, 15.7 mmol) in DCM (30 mL) was added TEA (3.2 g, 31.5 mmol) and tert-butoxycarbonyl tert-butyl carbonate (5.16 g, 23.7 mmol) and the reaction mixture was stirred at 25° C. for 10 h. The mixture was concentrated under vacuum and the crude was purified by CombiFlash® (PE/EtOAc=2/1) to give tert-butyl (5-oxo-5,6-dihydro-2H-pyran-3-yl)carbamate (500 mg, 28% yield) as yellow oil. LCMS m/z=214.3 (M+H)+

### 4. Synthesis of tert-butyl (5-oxotetrahydro-2H-pyran-3-yl)carbamate

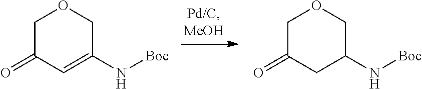 [SMILES: CC(C)(C)OC(=O)NC1=CC(=O)COC1 ; CC(C)(C)OC(=O)NC1COCC(=O)C1]

To a solution of tert-butyl (5-oxo-5,6-dihydro-2H-pyran-3-yl)carbamate (500 mg, 1.2 mmol) in MeOH (20 mL) was added Pd/C (374 mg, 10% purity) and the reaction stirred at 25° C. under H₂ (15 psi) for 3 h. The mixture was filtered and the filtrate evaporated under reduced pressure to give tert-butyl (5-oxotetrahydro-2H-pyran-3-yl)carbamate (500 mg, 79% yield) as white solid. LCMS m/z=216.3 (M+H)+

### 5. Synthesis of tert-butyl (5-hydroxytetrahydro-2H-pyran-3-yl)carbamate

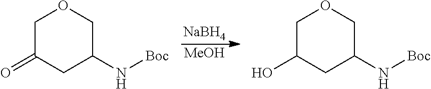 [SMILES: "MeOH" ; CC(C)(C)OC(=O)NC1COCC(=O)C1 ; "NaBH4" ; CC(C)(C)OC(=O)NC1COCC(O)C1]

To a solution of tert-butyl (5-oxo-5,6-dihydro-2H-pyran-3-yl)carbamate (500 mg, 2.32 mmol) in MeOH (20 mL) was added NaBH₄ (176 mg, 4.65 mmol) and the reaction stirred at 25° C. for 1 h. The mixture was quenched with water (5 mL). The filtrate was evaporated under reduced pressure to give tert-butyl (5-hydroxytetrahydro-2H-pyran-3-yl)carbamate (500 mg, crude) as colorless oil. LCMS m/z=218.3 (M+H)+

### 6. Synthesis of rac-tert-butyl ((3R,5S)-5-((6-(1-methyl-1H-pyrazol-4-yl)pyrazolo[1,5-a]pyrazin-4-yl)oxy)tetrahydro-2H-pyran-3-yl)carbamate and rac-tert-butyl ((3R,5R)-5-((6-(1-methyl-1H-pyrazol-4-yl)pyrazolo[1,5-a]pyrazin-4-yl)oxy)tetrahydro-2H-pyran-3-yl)carbamate

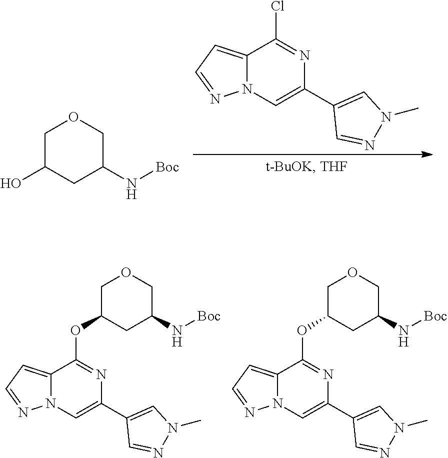 [SMILES: CC(C)(C)OC(=O)NC1COCC(O)C1 ; Cn1cc(-c2cn3nccc3c(Cl)n2)cn1 ; Cn1cc(-c2cn3nccc3c(O[C@H]3COC[C@@H](NC(=O)OC(C)(C)C)C3)n2)cn1 ; Cn1cc(-c2cn3nccc3c(O[C@@H]3COC[C@@H](NC(=O)OC(C)(C)C)C3)n2)cn1]

To a solution of tert-butyl (5-hydroxytetrahydro-2H-pyran-3-yl)carbamate (250 mg, 1.15 mmol) and 4-chloro-6-(1-methyl-1H-pyrazol-4-yl)pyrazolo[1,5-a]pyrazine (Intermediate A, 350 mg, 1.50 mmol) in THF (30 mL) was added t-BuONa (553 mg, 5.75 mmol) and the reaction mixture was stirred at 75° C. for 2 h. The reaction was quenched with water (10 mL), extracted with EtOAc (10 mL×3), the combined organic layer was washed with H₂O (10 mL) and brine (10 mL), dried over Na₂SO₄, filtered and the filtrate was concentrated in vacuo. The crude product was purified by prep-HPLC (Column Gemini C18 150×30 mm×4 um; Condition water (0.225% FA)-MeCN Begin B 35, End B 65 Gradient Time (min) 11, Flow Rate (mL/min) 30) to give

Peak 1, rac-tert-butyl ((3R,5S)-5-((6-(1-methyl-1H-pyrazol-4-yl)pyrazolo[1,5-a]pyrazin-4-yl)oxy)tetrahydro-2H-pyran-3-yl)carbamate (80 mg, 15% yield, 90% purity) as a white solid. 1HNMR: (500 MHz, MeOH-d₆) δ=8.46 (s, 1H), 8.11 (s, 1H), 8.01-7.85 (m, 2H), 6.86 (s, 1H), 5.53-5.41 (m, 1H), 4.10-4.06 (m, 1H), 3.95 (s, 3H), 3.90-3.86 (m, 1H), 3.81-3.69 (m, 2H), 3.8-3.45 (m, 1H), 2.42-2.40 (d, J=10 Hz, 1H), 2.08-1.98 (m, 1H), 1.38 (s, 9H).

Peak 2, rac-tert-butyl ((3R,5R)-5-((6-(1-methyl-1H-pyrazol-4-yl)pyrazolo[1,5-a]pyrazin-4-yl)oxy)tetrahydro-2H-pyran-3-yl)carbamate (60 mg, 11% yield). 1HNMR: (500 MHz, MeOH-d₆) δ=8.45 (s, 1H), 8.09 (s, 1H), 8.03-7.90 (m, 2H), 6.90 (s, 1H), 5.62 (s, 1H), 4.11-3.98 (m, 2H), 3.94 (s, 4H), 3.80-3.75 (d, J=15.0 Hz, 1H), 3.29-3.23 (m, 1H), 2.39-2.34 (d, J=15 Hz, 1H), 1.99-1.90 (m, 1H), 1.43 (s, 9H). NOE was used to determine the stereochemistry of the products.

### 7. Synthesis of rac-tert-butyl methyl((3R,5S)-5-((6-(1-methyl-1H-pyrazol-4-yl)pyrazolo[1,5-a]pyrazin-4-yl)oxy)tetrahydro-2H-pyran-3-yl)carbamate

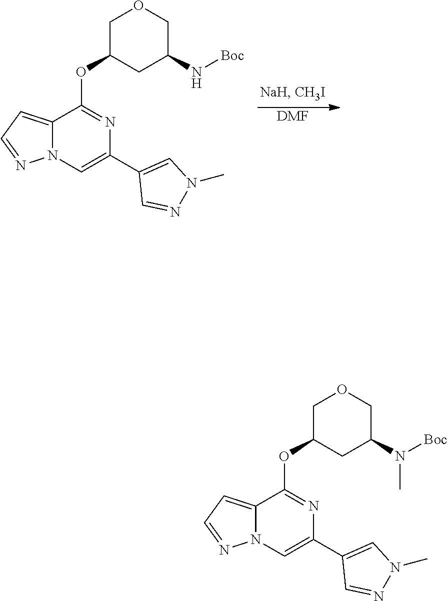

To a solution rac-tert-butyl ((3R,5S)-5-((6-(1-methyl-1H-pyrazol-4-yl)pyrazolo[1,5-a]pyrazin-4-yl)oxy)tetrahydro-2H-pyran-3-yl)carbamate (80 mg, 0.193 mmol) in DCM (15 mL) was added NaH (23 mg, 0.579 mmol, 60% purity) at 0° C. and the mixture stirred for 0.5 h. CH₃I (82 mg, 579 μmol) was added and the reaction stirred at 20° C. for 1 h. The reaction was quenched with water (20 mL), extracted with EtOAc (20 mL×3), the combined organic layer was washed with H₂O (20 mL×3) and brine (30 mL), dried over Na₂SO₄ and filtered. The filtrate was concentrated in vacuo to give rac-tert-butyl methyl((3R,5S)-5-((6-(1-methyl-1H-pyrazol-4-yl)pyrazolo[1,5-a]pyrazin-4-yl)oxy)tetrahydro-2H-pyran-3-yl)carbamate (65 mg, crude) as a white solid. LCMS m/z=429.2 (M+H)+

### 8. Synthesis of rac-(3R,5S)—N-methyl-5-((6-(1-methyl-1H-pyrazol-4-yl)pyrazolo[1,5-a]pyrazin-4-yl)oxy)tetrahydro-2H-pyran-3-amine hydrochloride

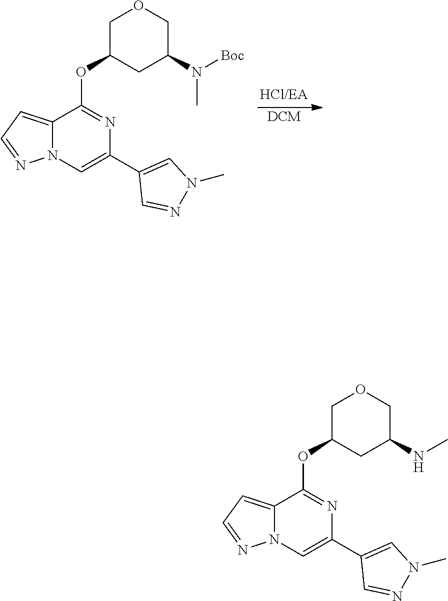

To a solution of rac-tert-butyl methyl((3R,5S)-5-((6-(1-methyl-1H-pyrazol-4-yl)pyrazolo[1,5-a]pyrazin-4-yl)oxy)tetrahydro-2H-pyran-3-yl)carbamate (65 mg, 0.152 mmol) in DCM (10 mL) was added HCl/EtOAc (4 M, 2 mL) and the reaction stirred at 25° C. for 1 h. The mixture was concentrated in vacuo to give rac-(3R,5S)—N-methyl-5-((6-(1-methyl-1H-pyrazol-4-yl)pyrazolo[1,5-a]pyrazin-4-yl)oxy)tetrahydro-2H-pyran-3-amine hydrochloride (65 mg, crude) as brown oil. LCMS m/z=329.2 (M+H)+

### 9. Synthesis of rac-N-methyl-N-((3R,5S)-5-((6-(1-methyl-1H-pyrazol-4-yl)pyrazolo[1,5-a]pyrazin-4-yl)oxy)tetrahydro-2H-pyran-3-yl)acrylamide

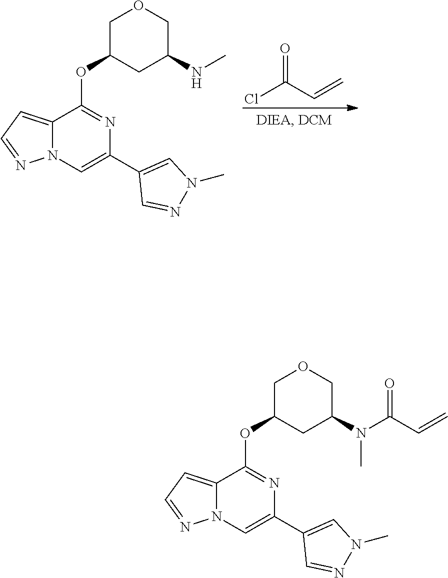

To a solution of rac-(3R,5S)—N-methyl-5-((6-(1-methyl-1H-pyrazol-4-yl)pyrazolo[1,5-a]pyrazin-4-yl)oxy)tetrahydro-2H-pyran-3-amine hydrochloride (65 mg, 0.198 mmol) and DIPEA (77 mg, 597 μmol) in DCM (10 mL) was added dropwise acryloyl chloride (23.3 mg, 257 μmol) at 0° C. The mixture was stirred at 25° C. for 0.5 h, then MeOH (3 mL) was added dropwise. The resulting mixture was stirred at 25° C. for 10 min, then concentrated in vacuo. The crude product was purified by prep HPLC (Column Welch Xtimate C18 150×25 mm×5 um; Condition water (0.05% NH₃H₂O+10 mM NH₄HCO₃)-MeCN Begin B 25, End B 55 Gradient Time (min) 10, FlowRate (mL/min) 25) to give rac-N-methyl-N-((3R,5S)-5-((6-(1-methyl-1H-pyrazol-4-yl)pyrazolo[1,5-a]pyrazin-4-yl)oxy)tetrahydro-2H-pyran-3-yl)acrylamide (52 mg, 69% yield) as a white solid. LCMS m/z=405.0 (M+Na)⁺; 1HNMR (500 MHz, DMSO-d₆) δ=8.78 (s, 1H), 8.23 (s, 1H), 8.04-7.98 (m, 2H), 6.98-6.69 (m, 2H), 6.16-6.05 (m, 1H), 5.69-5.47 (m, 1H), 5.58-5.37 (m, 1H), 4.70-4.13 (m, 2H), 3.89 (s, 3H), 3.82-3.68 (m, 1H), 3.65-3.42 (m, 2H), 3.03-2.85 (m, 3H), 2.42-2.28 (m, 1H), 2.14-2.08 (m, 1H).

### Example 97. rac-N-methyl-N-((3R,5R)-5-((6-(1-methyl-1H-pyrazol-4-yl)pyrazolo[1,5-a]pyrazin-4-yl)oxy)tetrahydro-2H-pyran-3-yl)acrylamide

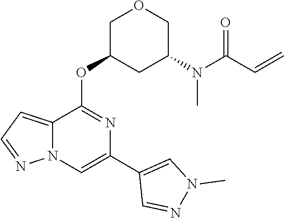 [SMILES: C=CC(=O)N(C)[C@H]1COC[C@H](Oc2nc(-c3cnn(C)c3)cn3nccc23)C1]

### 1. Synthesis of tert-butyl methyl((trans)-5-((6-(1-methyl-1H-pyrazol-4-yl)pyrazolo[1,5-a]pyrazin-4-yl)oxy)tetrahydro-2H-pyran-3-yl)carbamate

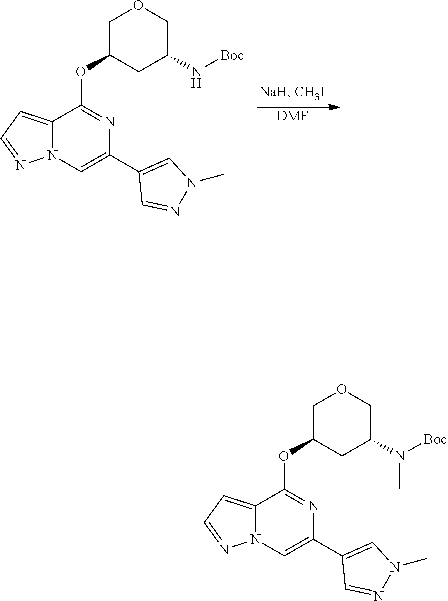

rac-tert-butyl methyl((3R,5R)-5-((6-(1-methyl-1H-pyrazol-4-yl)pyrazolo[1,5-a]pyrazin-4-yl)oxy)tetrahydro-2H-pyran-3-yl)carbamate was obtained, 50 mg, crude, from rac-tert-butyl ((3R,5R)-5-((6-(1-methyl-1H-pyrazol-4-yl)pyrazolo[1,5-a]pyrazin-4-yl)oxy)tetrahydro-2H-pyran-3-yl)carbamate (step 6, Example 96) following the procedure described in step 7, Example 95. LCMS m/z=451.2 (M+Na)+

### 2. Synthesis of rac-(3R,5R)—N-methyl-5-((6-(1-methyl-1H-pyrazol-4-yl)pyrazolo[1,5-a]pyrazin-4-yl)oxy)tetrahydro-2H-pyran-3-amine hydrochloride

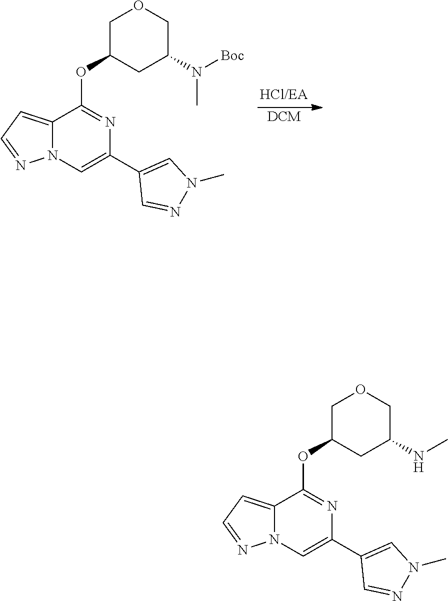

rac-(3R,5R)—N-methyl-5-((6-(1-methyl-1H-pyrazol-4-yl)pyrazolo[1,5-a]pyrazin-4-yl)oxy)tetrahydro-2H-pyran-3-amine hydrochloride was obtained as a brown oil, 50 mg, crude, from rac-tert-butyl methyl((3R,5R)-5-((6-(1-methyl-1H-pyrazol-4-yl)pyrazolo[1,5-a]pyrazin-4-yl)oxy)tetrahydro-2H-pyran-3-yl)carbamate hydrochloride following the procedure described in step 8, Example 96. LCMS m/z=329.2 (M+H)+

### 3. Synthesis of rac-N-methyl-N-((3R,5R)-5-((6-(1-methyl-1H-pyrazol-4-yl)pyrazolo[1,5-a]pyrazin-4-yl)oxy)tetrahydro-2H-pyran-3-yl)acrylamide

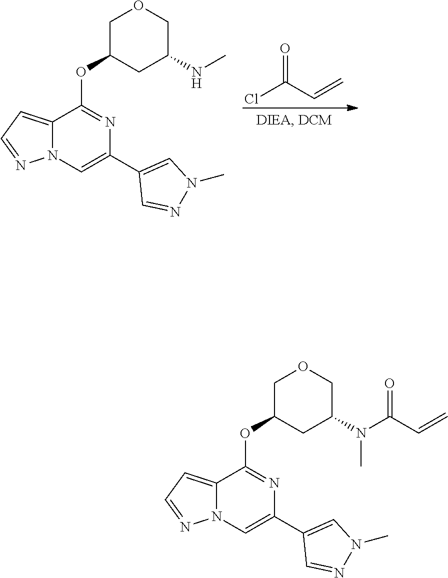

rac-N-methyl-N-((3R,5R)-5-((6-(1-methyl-1H-pyrazol-4-yl)pyrazolo[1,5-a]pyrazin-4-yl)oxy)tetrahydro-2H-pyran-3-yl)acrylamide was obtained as a white solid, 54 mg, 93% yield from rac-tert-butyl methyl((3R,5R)-5-((6-(1-methyl-1H-pyrazol-4-yl)pyrazolo[1,5-a]pyrazin-4-yl)oxy)tetrahydro-2H-pyran-3-yl)carbamate hydrochloride, following the procedure described in step 9, Example 96. LCMS m/z=405.0 (M+H)+1HNMR (500 MHz, DMSO-d₆) δ=8.77 (s, 1H), 8.19 (s, 1H), 8.12-7.88 (m, 2H), 6.95-6.79 (m, 1H), 6.76-6.68 (m, 1H), 6.15-5.94 (m, 1H), 5.72-5.58 (m, 2H), 4.85-4.36 (m, 1H), 4.10-4.08 (m, 1H), 3.88 (s, 3H), 3.79-3.53 (m, 3H), 2.99-2.83 (m, 3H), 2.34-2.09 (m, 2H).

### Example 98 and 99: N-(4-((6-(1-methyl-1H-pyrazol-4-yl)pyrazolo[1,5-a]pyrazin-4-yl)oxy)bicyclo[2.1.1]hexan-1-yl)acrylamide and N-methyl-N-(4-((6-(1-methyl-1H-pyrazol-4-yl)pyrazolo[1,5-a]pyrazin-4-yl)oxy)bicyclo[2.1.1]hexan-1-yl)acrylamide

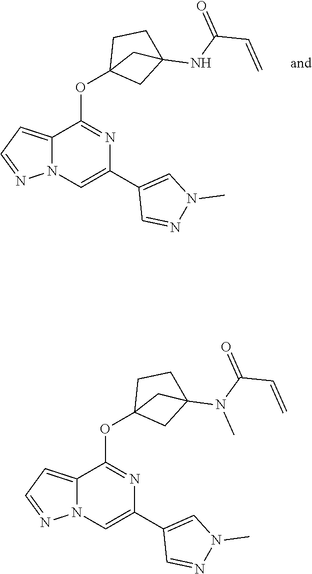 [SMILES: C=CC(=O)NC12CCC(Oc3nc(-c4cnn(C)c4)cn4nccc34)(C1)C2 ; C=CC(=O)N(C)C12CCC(Oc3nc(-c4cnn(C)c4)cn4nccc34)(C1)C2]

### 1. Synthesis of N-(4-((6-chloropyrazolo[1,5-a]pyrazin-4-yl)oxy)bicyclo[2.1.1]hexan-1-yl)acrylamide and N-(4-((6-chloropyrazolo[1,5-a]pyrazin-4-yl)oxy)bicyclo[2.1.1]hexan-1-yl)-N-methylacrylamide

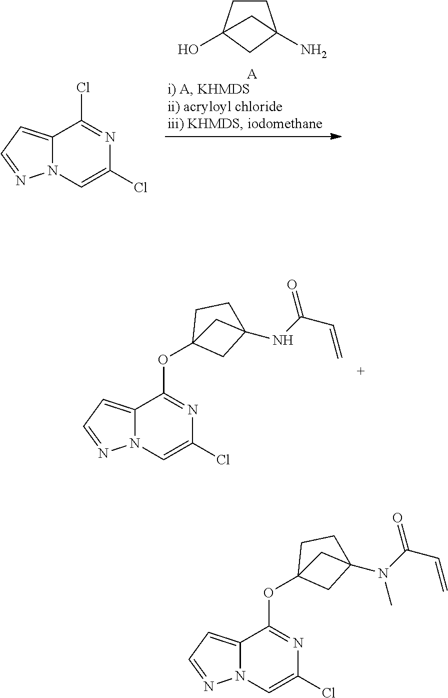

KHMDS (1M THF, 3.65 mL) was added a solution of 4-aminobicyclo[2.1.1]hexan-1-ol hydrochloride (300 mg, 2.01 mmol) in THF (10 mL) at RT within 5 min. After stirring for 10 min at that temperature a solution of 4,6-dichloropyrazolo[1,5-a]pyrazine (343 mg, 1.82 mmol) in THF (4 mL) was added dropwise within 5 min. After 15 min acryloyl chloride (150 μL, 1.82 mmol) was added dropwise to the vigorously stirred dark brown reaction mixture at RT and stirring was continued for another 30 min. Additional KHMDS (1M THF, 3.65 mL) was added to the reaction mixture followed by the dropwise addition of iodomethane (140 μL, 2.28 mmol). After stirring for an additional 60 min at RT the reaction mixture was diluted by the addition of EtOAc (10 mL) and sat aq. NaHCO₃ (10 mL). The biphasic mixture was vigorously stirred for 15 min and the organic phase was separated and washed with water (10 mL), brine (10 mL), dried (Na₂SO₄) and evaporated to dryness in vacuo. The residue was purified by column chromatography (24 g SiO₂, 0-100% EtOH:EtOAc (2% NH₄OH)1:3 in heptane) to afford an inseparable 4:1 mixture of N-(4-((6-chloropyrazolo[1,5-a]pyrazin-4-yl)oxy)bicyclo[2.1.1]hexan-1-yl)-N-methylacrylamide and N-(4-((6-chloropyrazolo[1,5-a]pyrazin-4-yl)oxy)bicyclo[2.1.1]hexan-1-yl)acrylamide as an orange gum (353 mg). LCMS m/z=333.1 (M+H)⁺, LCMS m/z=319.0 (M+H)⁺.

### 2. Synthesis of N-(4-((6-(1-methyl-1H-pyrazol-4-yl)pyrazolo[1,5-a]pyrazin-4-yl)oxy)bicyclo[2.1.1]hexan-1-yl)acrylamide and N-methyl-N-(4-((6-(1-methyl-1H-pyrazol-4-yl)pyrazolo[1,5-a]pyrazin-4-yl)oxy)bicyclo[2.1.1]hexan-1-yl)acrylamide

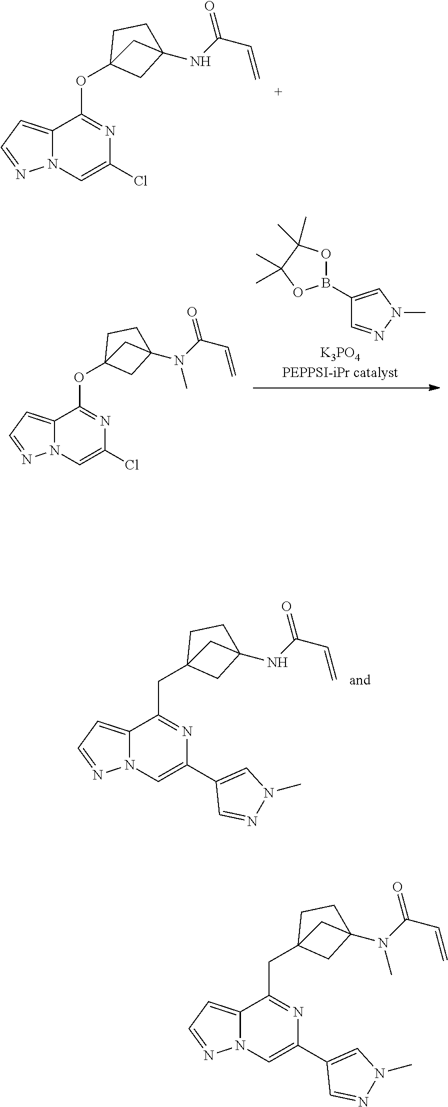

A mixture of a 4:1 mixture of 4:1 mixture of N-(4-((6-chloropyrazolo[1,5-a]pyrazin-4-yl)oxy)bicyclo[2.1.1]hexan-1-yl)-N-methylacrylamide and N-(4-((6-chloropyrazolo[1,5-a]pyrazin-4-yl)oxy)bicyclo[2.1.1]hexan-1-yl)acrylamide (353 mg, 1.06 mmol), 1-methyl-4-(4,4,5,5-tetramethyl-1,3,2-dioxaborolan-2-yl)-1H-pyrazole (441, 2.12 mmol), K₃PO₄ (675 mg, 3.18 mmol), and PePPSI-iPr catalyst (145 mg, 212 μmol) in dioxane (10 mL) and water (1 mL) was degassed by purging with nitrogen for 30 min. The resulting mixture was heated at reflux for 1 h, cooled to RT and diluted with EtOAc (25 mL). The separated organic phase was washed with water (20 mL) and brine (20 mL), dried over Na₂SO₄, filtered and concentrated. The residue was purified by column chromatography (24 g SiO₂, 20-100% EtOH:EtOAc (2% NH₄OH)1:3 in heptane) followed by preparative HPLC (XSelect CSH Prep C18 OBD 5 um 30×100 mm; Method: (A) 95% {H₂O}//(B) 5% {Acetonitrile} w/0.2% NH₄OH (initial conditions hold for 0.5 min) then a linear gradient to 5% (A)/75% (B) over 12 min (flow rate: 50 mL/min) to afford:

### Example 98 N-(4-((6-(1-methyl-1H-pyrazol-4-yl)pyrazolo[1,5-a]pyrazin-4-yl)oxy)bicyclo[2.1.1]hexan-1-yl)acrylamide (13 mg, 3% yield). LCMS m/z=365.1 (M+H)⁺. ¹H NMR (500 MHz, MeOH-d₄) δ ppm 8.44 (s, 1H), 8.12 (s, 1H), 7.87-8.01 (m, 2H), 6.77 (d, J=1.83 Hz, 1H), 6.18-6.31 (m, 2H), 5.65 (dd, J=4.58, 7.63 Hz, 1H), 3.96 (s, 3H), 2.47 (br s, 2H), 2.33-2.44 (m, 2H), 2.19-2.32 (m, 2H), 2.03-2.13 (m, 2H)

### Example 99 N-methyl-N-(4-((6-(1-methyl-1H-pyrazol-4-yl)pyrazolo[1,5-a]pyrazin-4-yl)oxy)bicyclo[2.1.1]hexan-1-yl)acrylamide (71 mg, 18% yield). LCMS m/z=401.1 (M+Na)⁺. ¹H NMR (500 MHz, MeOH-d₄) δ ppm 8.37 (s, 1H), 8.00 (s, 1H), 7.79-7.95 (m, 2H), 6.72 (d, J=1.83 Hz, 2H), 6.21 (dd, J=1.83, 17.09 Hz, 1H), 5.72 (dd, J=2.14, 10.68 Hz, 1H), 3.93 (s, 3H), 3.07 (br s, 3H), 2.64 (br s, 2H), 1.98-2.37 (m, 6H)

### Example 100. N-methyl-N-(5-((6-(1-methyl-1H-pyrazol-4-yl)pyrazolo[1,5-a]pyrazin-4-yl)oxy)-3-oxabicyclo[3.1.1]heptan-1-yl)but-2-ynamide

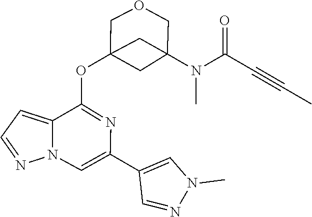 [SMILES: CC#CC(=O)N(C)C12COCC(Oc3nc(-c4cnn(C)c4)cn4nccc34)(C1)C2]

### 1. Synthesis of ethyl 1-((tert-butoxycarbonyl)amino)-3-methylenecyclobutane-1-carboxylate

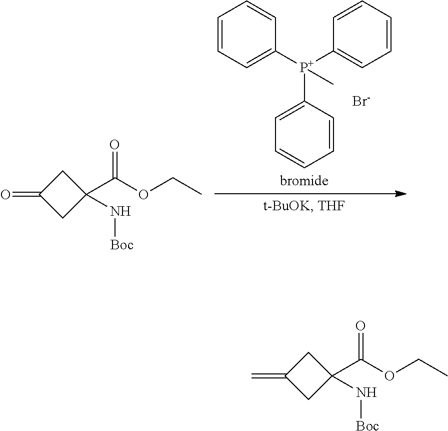 [SMILES: CCOC(=O)C1(NC(=O)OC(C)(C)C)CC(=O)C1 ; C[P+](c1ccccc1)(c1ccccc1)c1ccccc1 ; [Br-] ; C=C1CC(NC(=O)OC(C)(C)C)(C(=O)OCC)C1]

To a solution of methyltriphenylphosphonium (13.88 g, 38.9 mmol) in THF (250 mL) was added t-BuOK (1 M, 38.9 mL) and the mixture was stirred at 0° C. under N₂ for 30 min. Ethyl 1-((tert-butoxycarbonyl)amino)-3-oxocyclobutane-1-carboxylate (5 g, 19.4 mmol) was added and the reaction was stirred at 0° C. for 3 h. The reaction mixture was concentrated in vacuo and the crude was purified by column chromatography (PE: EtOAc=5:1 to 0:1) to give ethyl 1-((tert-butoxycarbonyl)amino)-3-methylenecyclobutane-1-carboxylate (1.6 g, 29% yield) as a white solid. LCMS m/z=256.3 (M+H)+

### 2. Synthesis of tert-butyl (1-(hydroxymethyl)-3-methylenecyclobutyl)carbamate

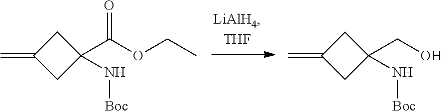 [SMILES: C=C1CC(NC(=O)OC(C)(C)C)(C(=O)OCC)C1 ; C=C1CC(CO)(NC(=O)OC(C)(C)C)C1]

To a solution of ethyl 1-((tert-butoxycarbonyl)amino)-3-methylenecyclobutane-1-carboxylate (1.5 g, 5.88 mmol) in THF (45 mL) was added LiAlH₄ (600 mg, 15.8 mmol) at 0° C. under N₂ and the reaction was stirred at 0° C. for 6 h. Water (2 mL) was added dropwise. The mixture was filtered and the filtrate was concentrated in vacuo. The crude was purified by column chromatography (PE: EtOAc=5:2 to 0:1) to give tert-butyl (1-(hydroxymethyl)-3-methylenecyclobutyl)carbamate (1.09 g, 78% yield) as a white solid. LCMS m/z=214.3 (M+H)+

### 3. Synthesis of tert-butyl (5-(hydroxymethyl)-1-oxaspiro[2.3]hexan-5-yl)carbamate

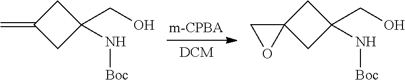 [SMILES: C=C1CC(CO)(NC(=O)OC(C)(C)C)C1 ; CC(C)(C)OC(=O)NC1(CO)CC2(CO2)C1]

The mixture of tert-butyl (1-(hydroxymethyl)-3-methylenecyclobutyl)carbamate (900 mg, 4.22 mmol) in DCM (20 mL) was added MCPBA (1.80 g, 8.87 mmol, 85% purity) at 20° C. The reaction was stirred at 20° C. for 5 h. The reaction solution was quenched by aq. Na₂SO₃ (20 mL), water (30 mL) was added and the mixture was extracted with DCM (3×30 mL). The combined organics were washed with sat.aq. NaHCO₃ (30 mL), sat.aq. Na₂SO₃ (30 mL) and brine (30 mL), then dried over Na₂SO₄ and filtered. The filtrate was concentrated under vacuum to give tert-butyl (5-(hydroxymethyl)-1-oxaspiro[2.3]hexan-5-yl)carbamate (800 mg, crude) as yellow oil. LCMS m/z=230.3 (M+H)+

### 4. Synthesis of tert-butyl (5-hydroxy-3-oxabicyclo[3.1.1]heptan-1-yl)carbamate

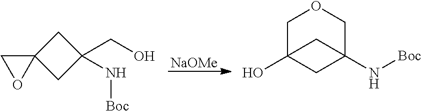 [SMILES: CC(C)(C)OC(=O)NC1(CO)CC2(CO2)C1 ; CC(C)(C)OC(=O)NC12COCC(O)(C1)C2 ; "NaOMe"]

To a solution of tert-butyl (5-(hydroxymethyl)-1-oxaspiro[2.3]hexan-5-yl)carbamate (800 mg, 3.49 mmol) in MeOH (20 mL) was added NaOMe (189 mg, 3.49 mmol) at 20° C. and the reaction was stirred for 12 h. HCl aq. was added to adjust pH=8 and the resulting mixture was extracted with DCM (20 mL×3), brine (3 mL), dried over Na₂SO₄, filtered and concentrated in vacuo. The crude was purified by column chromatography (PE:EtOAc=5:4 to 0:1) to give tert-butyl (5-hydroxy-3-oxabicyclo[3.1.1]heptan-1-yl)carbamate (300 mg, 30% yield.) as a white solid. LCMS m/z=230.3 (M+H)+

### 5. Synthesis of tert-butyl (5-((6-(1-methyl-1H-pyrazol-4-yl)pyrazolo[1,5-a]pyrazin-4-yl)oxy)-3-oxabicyclo[3.1.1]heptan-1-yl)carbamate

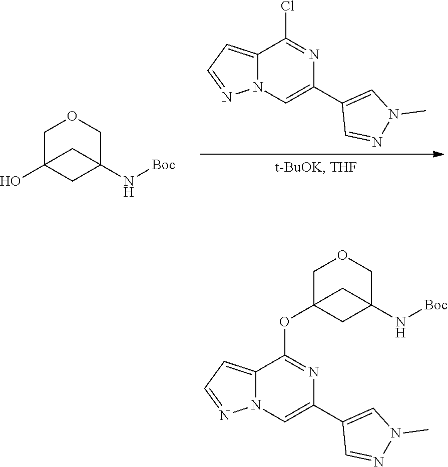 [SMILES: CC(C)(C)OC(=O)NC12COCC(O)(C1)C2 ; Cn1cc(-c2cn3nccc3c(Cl)n2)cn1 ; Cn1cc(-c2cn3nccc3c(OC34COCC(NC(=O)OC(C)(C)C)(C3)C4)n2)cn1]

To a solution of tert-butyl (5-hydroxy-3-oxabicyclo[3.1.1]heptan-1-yl)carbamate (270 mg, 1.18 mmol) in THF (50 mL) was added t-BuOK (396 mg, 3.53 mmol) and 4-chloro-6-(1-methyl-1H-pyrazol-4-yl)pyrazolo[1,5-a]pyrazine (Intermediate A, 275 mg, 1.18 mmol) and the reaction was stirred at 20° C. for 1 h. Further t-BuOK (264 mg, 2.36 mmol) was added and the reaction was stirred at 20° C. for 2 h. The reaction mixture was concentrated in vacuo and the crude product was purified by prep HPLC (Column: Welch Xtimate C18 150×25 mm×5 pm; Condition: water (10 mM NH₄HCO₃)-MeCN, Begin B 39, End B 69, Gradient Time (min) 10, Flow Rate (mL/min) 25) to give tert-butyl (5-((6-(1-methyl-1H-pyrazol-4-yl)pyrazolo[1,5-a]pyrazin-4-yl)oxy)-3-oxabicyclo[3.1.1]heptan-1-yl)carbamate (230 mg, 44% yield) as a white solid. LCMS m/z=427.3 (M+H)+

### 6. Synthesis of tert-butyl methyl(5-((6-(1-methyl-1H-pyrazol-4-yl)pyrazolo[1,5-a]pyrazin-4-yl)oxy)-3-oxabicyclo[3.1.1]heptan-1-yl)carbamate

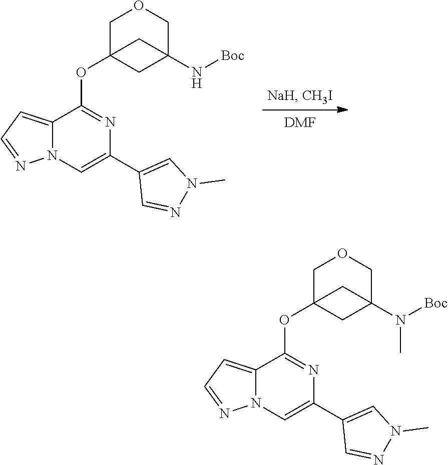

To a solution of tert-butyl (5-((6-(1-methyl-1H-pyrazol-4-yl)pyrazolo[1,5-a]pyrazin-4-yl)oxy)-3-oxabicyclo[3.1.1]heptan-1-yl)carbamate (200 mg, 469 μmol) in DMF (5 mL) was added NaH (47 mg, 1.17 mmol, 60% purity) at 0° C. and the mixture stirred for 20 min, then cooled to 0° C. CH₃I (67 mg, 469 μmol) was added and the reaction stirred at 0° C. for 2 h. The mixture was concentrated in vacuo, then lyophilized and purified by column chromatography (PE: EtOAc=1:1 to 0:1) to give tert-butyl methyl(5-((6-(1-methyl-1H-pyrazol-4-yl)pyrazolo[1,5-a]pyrazin-4-yl)oxy)-3-oxabicyclo[3.1.1]heptan-1-yl)carbamate (200 mg, 97% yield) as a white solid. LCMS m/z=441.2 (M+H)+

### 7. Synthesis of N-methyl-5-((6-(1-methyl-1H-pyrazol-4-yl)pyrazolo[1,5-a]pyrazin-4-yl)oxy)-3-oxabicyclo[3.1.1]heptan-1-amine hydrochloride

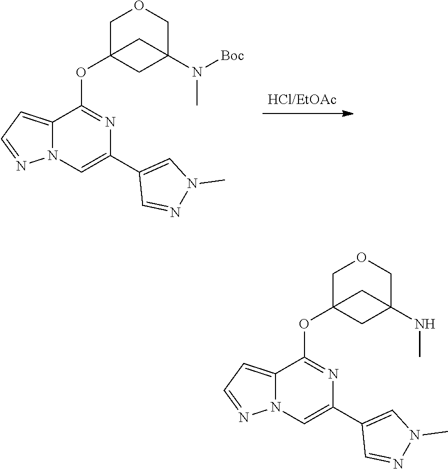 [SMILES: CN(C(=O)OC(C)(C)C)C12COCC(Oc3nc(-c4cnn(C)c4)cn4nccc34)(C1)C2 ; CNC12COCC(Oc3nc(-c4cnn(C)c4)cn4nccc34)(C1)C2]

To a solution of tert-butyl methyl(5-((6-(1-methyl-1H-pyrazol-4-yl)pyrazolo[1,5-a]pyrazin-4-yl)oxy)-3-oxabicyclo[3.1.1]heptan-1-yl)carbamate (190 mg, 431 μmol) in DCM (10 mL) was added HCl/EtOAc (4 M, 10 mL) and the reaction was stirred at 20° C. for 1 h. The mixture was concentrated under vacuum to give N-methyl-5-((6-(1-methyl-1H-pyrazol-4-yl)pyrazolo[1,5-a]pyrazin-4-yl)oxy)-3-oxabicyclo[3.1.1]heptan-1-amine hydrochloride (150 mg, crude) as yellow oil. LCMS m/z=341.2 (M+H)+

### 8. Synthesis of N-methyl-N-(5-((6-(1-methyl-1H-pyrazol-4-yl)pyrazolo[1,5-a]pyrazin-4-yl)oxy)-3-oxabicyclo[3.1.1]heptan-1-yl)but-2-ynamide

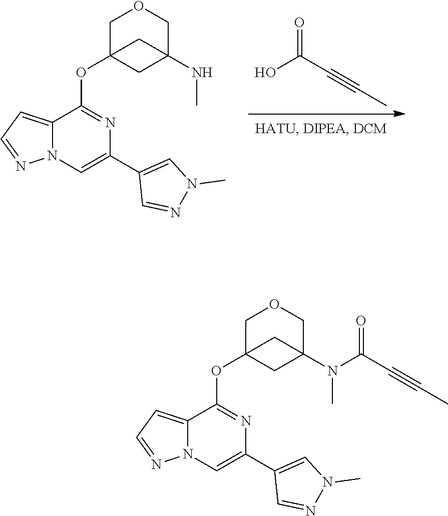

N-Methyl-N-(5-((6-(1-methyl-1H-pyrazol-4-yl)pyrazolo[1,5-a]pyrazin-4-yl)oxy)-3-oxabicyclo[3.1.1]heptan-1-yl)but-2-ynamide was obtained as a white solid, 63 mg, 74% yield from N-methyl-5-((6-(1-methyl-1H-pyrazol-4-yl)pyrazolo[1,5-a]pyrazin-4-yl)oxy)-3-oxabicyclo[3.1.1]heptan-1-amine hydrochloride, following the procedure described in step 4, Example 95. LCMS m/z=407.1 (M+H)+. 1H NMR (500 MHz, MeOH-d₄) δ ppm: 8.52-8.48 (m, 1H), 8.14-8.10 (m, 1H), 8.00-7.94 (m, 2H), 6.83-6.79 (m, 1H), 4.26-4.23 (m, 2H), 3.99-3.87 (m, 5H), 3.19-2.95 (m, 5H), 2.77-2.57 (m, 2H), 2.09-2.05 (m, 3H).

### Examples 101 & 102. N-cyclopropyl-N-((cis)-3-((6-(1-methyl-1H-pyrazol-4-yl)pyrazolo[1,5-a]pyrazin-4-yl)oxy)cyclobutyl)acrylamide and N-cyclopropyl-N-((trans)-3-((6-(1-methyl-1H-pyrazol-4-yl)pyrazolo[1,5-a]pyrazin-4-yl)oxy)cyclobutyl)acrylamide

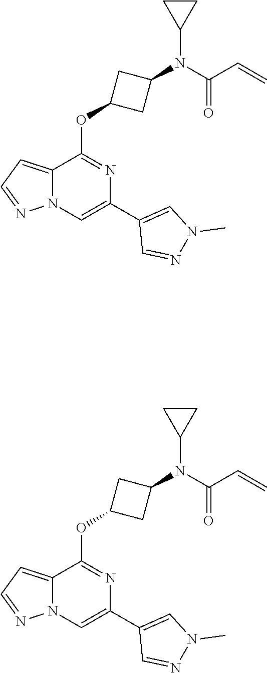 [SMILES: C=CC(=O)N(C1CC1)[C@H]1C[C@@H](Oc2nc(-c3cnn(C)c3)cn3nccc23)C1 ; C=CC(=O)N(C1CC1)[C@H]1C[C@H](Oc2nc(-c3cnn(C)c3)cn3nccc23)C1]

### 1. Synthesis of 3-(benzyloxy)-N-cyclopropylcyclobutan-1-amine

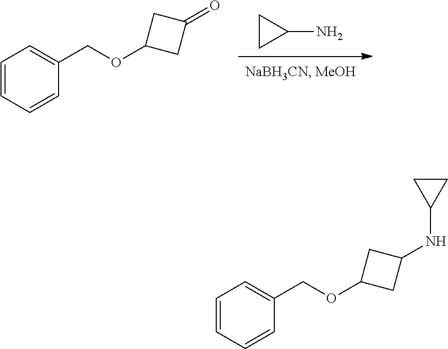

To a solution of 3-(benzyloxy)cyclobutan-1-one (700 mg, 3.97 mmol) in MeOH (20 mL) was added cyclopropylamine (2.27 g, 40 mmol) at 25° C. and the solution stirred for 0.5 h. NaBH₃CN (499 mg, 7.95 mmol) was added and the reaction stirred at 25° C. for 2 h. The mixture was concentrated under reduced pressure and the crude product was purified by silica gel chromatography eluting with PE/EtOAc (1/0 to 2/1) to give 3-(benzyloxy)cyclobutan-1-one (400 mg, 44% yield) as colorless oil. LCMS m/z=218.3 (M+H)+

### 2. Synthesis of tert-butyl (3-(benzyloxy)cyclobutyl)(cyclopropyl)carbamate

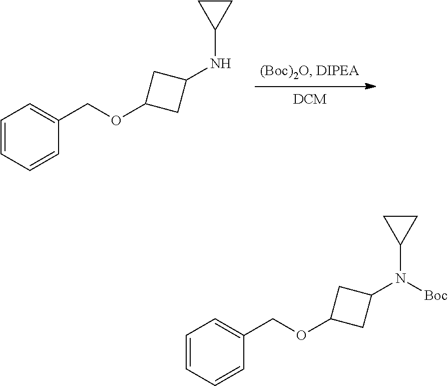

To a solution of 3-(benzyloxy)cyclobutan-1-one (300 mg, 1.38 mmol) in THF (40 mL) was added DIPEA (357 mg, 2.76 mmol) at 25° C. followed by di-tert-butyl dicarbonate (603 mg, 2.76 mmol) and the reaction was stirred at 25° C. for 0.5 h. The mixture was concentrated under vacuum and the crude product was purified by prep-HPLC (Welch Xtimate C18 150×25 mm×5 um, water (10 mM NH₄HCO₃)-MeCN as mobile phase, from 55-85%, Flow Rate (mL/min): 25) to give tert-butyl (3-(benzyloxy)cyclobutyl)(cyclopropyl)carbamate (410 mg, 94% yield) as orange oil. LCMS m/z=318.3 (M+H)+

### 3. Synthesis of tert-butyl cyclopropyl(3-hydroxycyclobutyl)carbamate

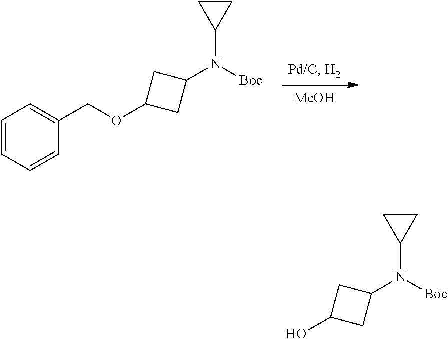 [SMILES: "MeOH" ; CC(C)(C)OC(=O)N(C1CC1)C1CC(OCc2ccccc2)C1 ; "Pd/C, H2" ; CC(C)(C)OC(=O)N(C1CC1)C1CC(O)C1]

To a solution of tert-butyl (3-(benzyloxy)cyclobutyl)(cyclopropyl)carbamate (410 mg, 1.29 mmol) in MeOH (30 mL) was added Pd/C (1.5 g) at 25° C., the suspension was degassed under vacuum and purged with H₂ (3×). The reaction mixture was stirred at 25° C. for 12 h then filtered. The filtrate was evaporated under reduced pressure to give tert-butyl cyclopropyl(3-hydroxycyclobutyl)carbamate that was used without further purification. LCMS m/z=228.3 (M+H)+.

### 4. Synthesis of tert-butyl cyclopropyl(3-((6-(1-methyl-1H-pyrazol-4-yl)pyrazolo[1,5-a]pyrazin-4-yl)oxy)cyclobutyl)carbamate

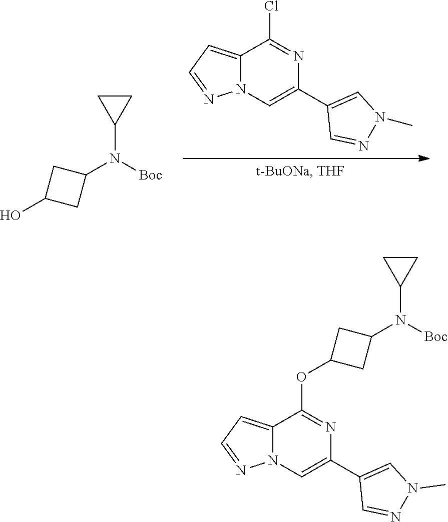

To a solution of 4-chloro-6-(1-methyl-1H-pyrazol-4-yl)pyrazolo[1,5-a]pyrazine (Intermediate A, 257 mg, 1.10 mmol) in THF (20 mL) was added t-BuONa (317 mg, 3.30 mmol) then tert-butyl cyclopropyl(3-hydroxycyclobutyl)carbamate (250 mg, 1.10 mmol) and the reaction was stirred at 70° C. for 2 h. The mixture was filtered and the filtrate concentrated under vacuum. The crude product was purified by column chromatography on silica gel (PE/EtOAc=1/0 to 3/2) to give tert-butyl cyclopropyl(3-((6-(1-methyl-1H-pyrazol-4-yl)pyrazolo[1,5-a]pyrazin-4-yl)oxy)cyclobutyl)carbamate (450 mg, 85% yield) as yellow solid. LCMS m/z=425.4 (M+H)+

### 5. Synthesis of N-cyclopropyl-3-((6-(1-methyl-1H-pyrazol-4-yl)pyrazolo[1,5-a]pyrazin-4-yl)oxy)cyclobutan-1-amine hydrochloride

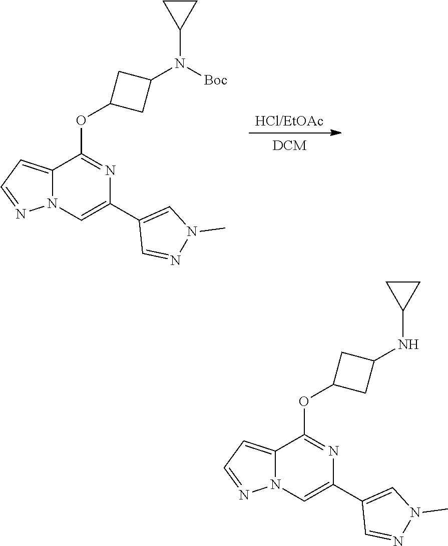

A solution of tert-butyl cyclopropyl(3-((6-(1-methyl-1H-pyrazol-4-yl)pyrazolo[1,5-a]pyrazin-4-yl)oxy)cyclobutyl)carbamate (430 mg, 1.01 mmol) in HCl/EtOAc (15 mL) and DCM (15 mL) was stirred at 25° C. for 0.5 h. The mixture was filtered and concentrated under vacuum to give N-cyclopropyl-3-((6-(1-methyl-1H-pyrazol-4-yl)pyrazolo[1,5-a]pyrazin-4-yl)oxy)cyclobutan-1-amine hydrochloride which was used for the next step without further purification. LCMS m/z=325.3 (M+H)+

### 6. Synthesis of N-cyclopropyl-N-(3-((6-(1-methyl-1H-pyrazol-4-yl)pyrazolo[1,5-a]pyrazin-4-yl)oxy)cyclobutyl)acrylamide

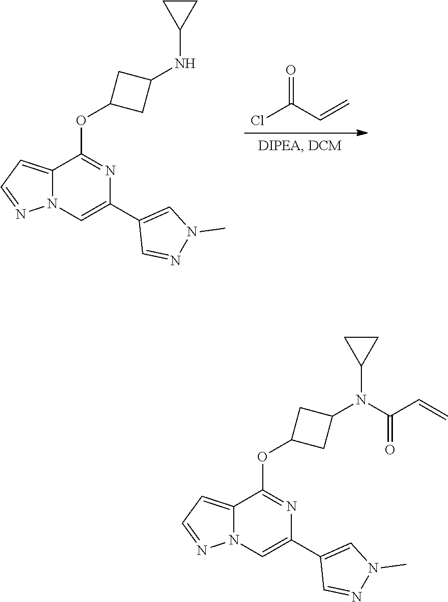

To a solution of N-cyclopropyl-3-((6-(1-methyl-1H-pyrazol-4-yl)pyrazolo[1,5-a]pyrazin-4-yl)oxy)cyclobutan-1-amine hydrochloride (315 mg, 0.97 mmol) in DCM (30 mL) was added DIPEA (251 mg, 1.94 mmol), the solution cooled to 0° C., acryloyl chloride (88 mg, 0.97 mmol) was added and the reaction stirred at 0° C. for 0.5 h. The mixture was concentrated under vacuum and the crude product was purified by prep-HPLC (Welch Xtimate C18 150×25 mm×5 um, water (10 mM NH₄HCO₃)-MeCN as mobile phase, from 30-60%, Flow Rate (mL/min): 25) to give N-cyclopropyl-N-(3-((6-(1-methyl-1H-pyrazol-4-yl)pyrazolo[1,5-a]pyrazin-4-yl)oxy)cyclobutyl)acrylamide (200 mg, 55% yield) as a white solid. LCMS m/z=379.1 (M+H)+

### 7. Synthesis of N-cyclopropyl-N-((cis)-3-((6-(1-methyl-1H-pyrazol-4-yl)pyrazolo[1,5-a]pyrazin-4-yl)oxy)cyclobutyl)acrylamide and N-cyclopropyl-N-((trans)-3-((6-(1-methyl-1H-pyrazol-4-yl)pyrazolo[1,5-a]pyrazin-4-yl)oxy)cyclobutyl)acrylamide

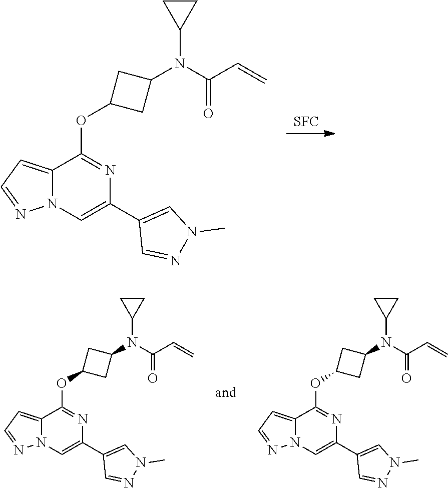 [SMILES: C=CC(=O)N(C1CC1)C1CC(Oc2nc(-c3cnn(C)c3)cn3nccc23)C1 ; "SFC" ; C=CC(=O)N(C1CC1)[C@H]1C[C@@H](Oc2nc(-c3cnn(C)c3)cn3nccc23)C1 ; C=CC(=O)N(C1CC1)[C@H]1C[C@H](Oc2nc(-c3cnn(C)c3)cn3nccc23)C1]

N-Cyclopropyl-N-(3-((6-(1-methyl-1H-pyrazol-4-yl)pyrazolo[1,5-a]pyrazin-4-yl)oxy)cyclobutyl)acrylamide (200 mg, 0.53 mmol) was purified by SFC (column: DAICEL CHIRALCEL OJ-H (250 mm×30 mm×5 μm), 25% (0.1% NH₃H₂O/EtOH) as mobile phase, Flow Rate (mL/min): 60) to give:

### Example 101: The first eluting diastereoisomer, peak 1 (99.8 mg, 50% yield) as a white solid, LCMS m/z=379.2 (M+H)+. 1H NMR (500 MHz, DMSO-d₆) δ ppm=8.77 (s, 1H), 8.22 (s, 1H), 8.03 (s, 2H), 6.93-6.85 (m, 2H), 6.12-6.08 (m, 1H), 5.68-5.64 (m, 1H), 5.17-5.14 (m, 1H), 4.01-3.97 (m, 1H), 3.91 (s, 3H), 3.04-3.01 (m, 2H), 2.83 (s, 1H), 2.47-2.44 (m, 2H), 0.91 (d, J=5.5 Hz, 2H), 0.65-0.62 (m, 2H)

### Example 102: And the second eluting diastereoisomer, peak 2 (45.8 mg, 22% yield) as a white solid. LCMS m/z=379.2 (M+H)+. 1H NMR: (500 MHz, DMSO-d₆) δ=8.77 (s, 1H), 8.22 (s, 1H), 8.16-7.96 (m, 2H), 6.95-6.89 (m, 2H), 6.14-6.09 (m, 1H), 5.69-5.68 (m, 1H), 5.67-5.54 (m, 1H), 4.56-4.48 (m, 1H), 3.89 (s, 3H), 2.95-2.91 (m, 2H), 2.86 (s, 1H), 2.61-2.56 (m, 2H), 0.96-0.91 (m, 2H), 0.68-0.64 (m, 2H)

The absolute stereochemistry of the product in each peak was not assigned.

### Examples 103 & 104. N-((1s,3s)-3-cyclopropyl-3-((6-(1-methyl-1H-pyrazol-4-yl)pyrazolo[1,5-a]pyrazin-4-yl)oxy)cyclobutyl)-N-methylacrylamide and N—((1r,3r)-3-cyclopropyl-3-((6-(1-methyl-1H-pyrazol-4-yl)pyrazolo[1,5-a]pyrazin-4-yl)oxy)cyclobutyl)-N-methylacrylamide

### 1. Synthesis of tert-butyl (3-cyclopropyl-3-hydroxycyclobutyl)(methyl)carbamate

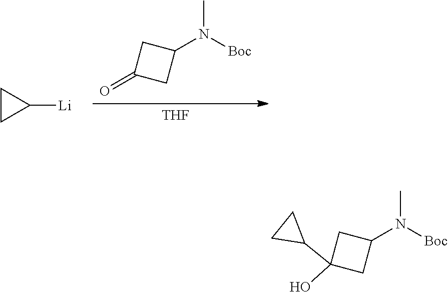

To a solution of cyclopropyl bromide (640 mg, 5.29 mmol) in diethyl ether (8 mL) was added Sec-BuLi (1.3 M, 3.05 mL) at −70° C. under N₂ and the mixture was stirred at −70° C. under N₂ for 2 h. The mixture was used for the next step directly without further purification.

To a solution of tert-butyl methyl(3-oxocyclobutyl)carbamate (200 mg, 1.0 mmol) in THF (5 mL) was added cyclopropyl lithium solution at −70° C. under N₂ and the reaction stirred at 20° C. under N₂ for 12 h. The mixture was carefully quenched with MeOH (15 mL) then concentrated in vacuo. The crude product was purified by Prep-TLC (PE/EtOAc=1/1) to give tert-butyl (3-cyclopropyl-3-hydroxycyclobutyl)(methyl)carbamate (180 mg, 67% yield) as yellow oil. LCMS m/z=242.2 (M+H)+

### 2. Synthesis of tert-butyl (3-((6-chloropyrazolo[1,5-a]pyrazin-4-yl)oxy)-3-cyclopropylcyclobutyl)(methyl)carbamate

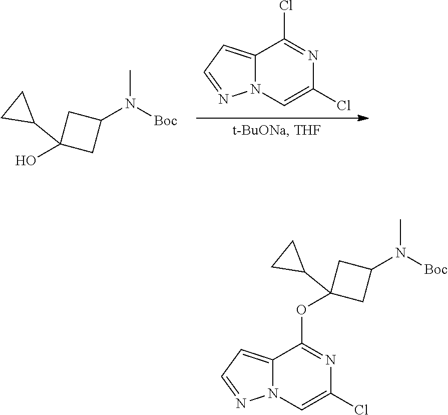

A solution of tert-butyl (3-cyclopropyl-3-hydroxycyclobutyl)(methyl)carbamate (300 mg, 1.24 mmol) in THF (30 mL) was added t-BuONa (358 mg, 3.73 mmol) at 20° C. 4,6-Dichloropyrazolo[1,5-a]pyrazine (234 mg, 1.24 mmol) was added slowly to the mixture at 0° C. and the reaction stirred for 30 mins. The mixture was concentrated in vacuo and the crude was purified by column chromatography on silica gel (PE/EtOAc=I/O to 1/1) to give tert-butyl (3-((6-chloropyrazolo[1,5-a]pyrazin-4-yl)oxy)-3-cyclopropylcyclobutyl)(methyl)carbamate (350 mg, 68% yield) as yellow oil. LCMS m/z=393.2 (M+H)+

### 3. Synthesis of tert-butyl (3-cyclopropyl-3-((6-(1-methyl-1H-pyrazol-4-yl)pyrazolo[1,5-a]pyrazin-4-yl)oxy)cyclobutyl)(methyl)carbamate

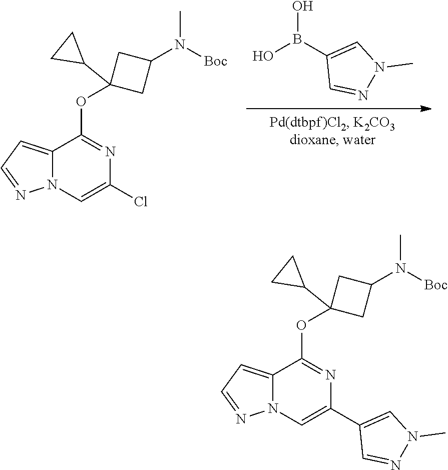

To a solution of tert-butyl (3-((6-chloropyrazolo[1,5-a]pyrazin-4-yl)oxy)-3-cyclopropylcyclobutyl)(methyl)carbamate (300 mg, 764 μmol) in dioxane (6 mL) and water (1.2 mL) was added K₂CO₃ (317 mg, 2.29 mmol) and (1-methyl-1H-pyrazol-4-yl)boronic acid (150 mg, 1.19 mmol) at 20° C. Pd(dtbpf)Cl₂ (100 mg, 153 μmol) was added and the reaction was stirred at 90° C. under N₂ for 2 h. The mixture was concentrated under vacuum and the crude product was purified by column chromatography on silica gel (PE/EtOAc=1/0 to 0/1) to give tert-butyl (3-cyclopropyl-3-((6-(1-methyl-1H-pyrazol-4-yl)pyrazolo[1,5-a]pyrazin-4-yl)oxy)cyclobutyl)(methyl)carbamate (300 mg, 89% yield) as yellow oil. ¹H NMR (400 MHz, MeOH-d₄) δ=8.43 (s, 1H), 8.06 (s, 1H), 7.98-7.85 (m, 2H), 6.83-6.69 (m, 1H), 4.37-4.13 (m, 1H), 3.95 (s, 3H), 2.82 (s, 3H), 2.77-2.66 (m, 2H), 2.66-2.55 (m, 2H), 1.55-1.32 (m, 10H), 0.63-0.55 (m, 4H).

### 4. Synthesis of 3-cyclopropyl-N-methyl-3-((6-(1-methyl-1H-pyrazol-4-yl)pyrazolo[1,5-a]pyrazin-4-yl)oxy)cyclobutan-1-amine

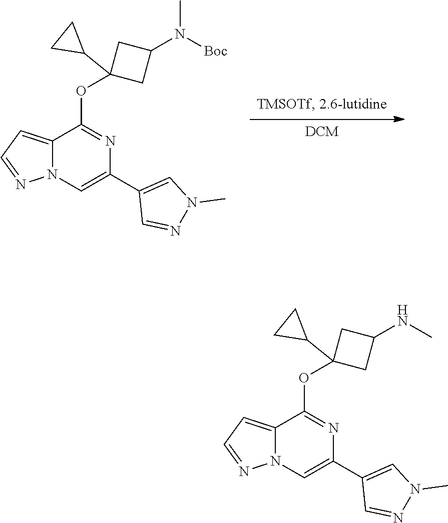

To a solution of tert-butyl (3-cyclopropyl-3-((6-(1-methyl-1H-pyrazol-4-yl)pyrazolo[1,5-a]pyrazin-4-yl)oxy)cyclobutyl)(methyl)carbamate (250 mg, 570 μmol) in DCM (15 mL) was added TMSOTf (634 mg, 2.85 mmol) and 2,6-lutidine (611 mg, 5.70 mmol) at 0° C. and the reaction stirred at 20° C. for 12 h. The mixture was concentrated under vacuum to give 3-cyclopropyl-N-methyl-3-((6-(1-methyl-1H-pyrazol-4-yl)pyrazolo[1,5-a]pyrazin-4-yl)oxy)cyclobutan-1-amine (200 mg, crude) as yellow oil, which was used for the next step directly without further purification. LCMS m/z=339.2 (M+H)+

### 5. Synthesis of N-(3-cyclopropyl-3-((6-(1-methyl-1H-pyrazol-4-yl)pyrazolo[1,5-a]pyrazin-4-yl)oxy)cyclobutyl)-N-methylacrylamide

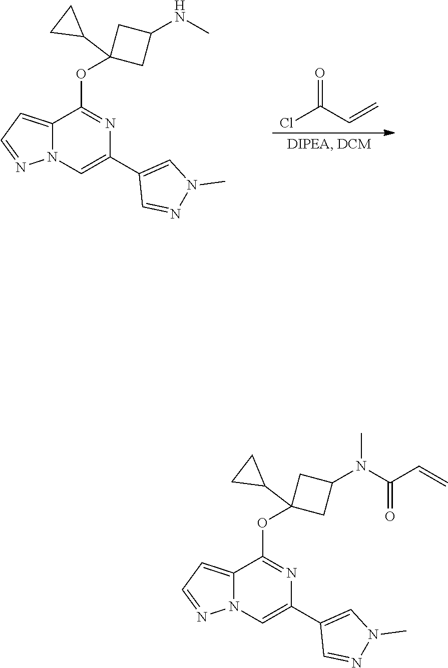

To a solution of 3-cyclopropyl-N-methyl-3-((6-(1-methyl-1H-pyrazol-4-yl)pyrazolo[1,5-a]pyrazin-4-yl)oxy)cyclobutan-1-amine (200 mg, 591 μmol) in DCM (20 mL) was added DIPEA (382 mg, 2.96 mmol) and the solution cooled to 0° C. Acryloyl chloride (80 mg, 887 μmol) was added and the reaction stirred at 0° C. for 2 h. The mixture was poured into water (50 mL) and extracted with DCM (50 mL×3). The combined organic layers were washed with brine (100 mL), dried over Na₂SO₄, filtered and concentrated under vacuum to give the crude, which was purified by Prep-HPLC (Column: Welch Xtimate C18 150×25 mm×5 μm; Condition: water(10 mM NH₄HCO₃)-MeCN, Begin B 30, End B 60; Gradient Time (min): 10; Flow Rate (mL/min): 25) to give N-(3-cyclopropyl-3-((6-(1-methyl-1H-pyrazol-4-yl)pyrazolo[1,5-a]pyrazin-4-yl)oxy)cyclobutyl)-N-methylacrylamide (150 mg, 60% yield) as a yellow solid. LCMS m/z=415.1 (M+H)+

### 6. Synthesis of N-((1s,3s)-3-cyclopropyl-3-((6-(1-methyl-1H-pyrazol-4-yl)pyrazolo[1,5-a]pyrazin-4-yl)oxy)cyclobutyl)-N-methylacrylamide and N-((1r,3r)-3-cyclopropyl-3-((6-(1-methyl-1H-pyrazol-4-yl)pyrazolo[1,5-a]pyrazin-4-yl)oxy)cyclobutyl)-N-methylacrylamide

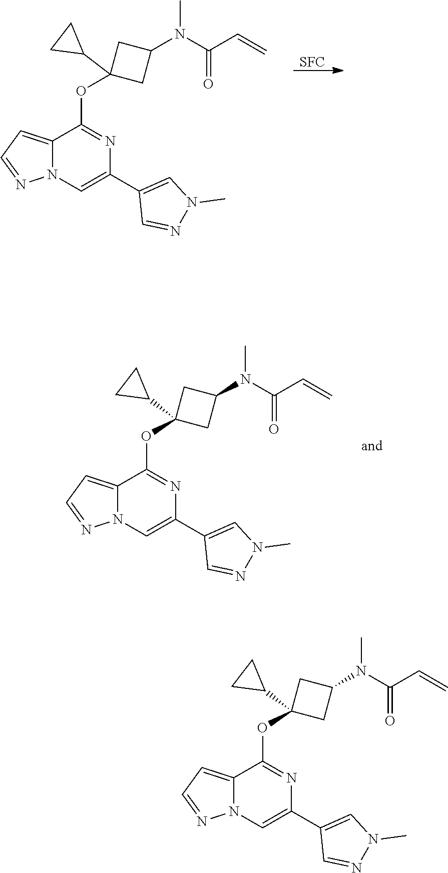

N-(3-Cyclopropyl-3-((6-(1-methyl-1H-pyrazol-4-yl)pyrazolo[1,5-a]pyrazin-4-yl)oxy)cyclobutyl)-N-methylacrylamide (150 mg, 382 μmol) was purified by SFC (column: DAICEL CHIRALPAK AD (250 mm×30 mm, 10 μm), (35% 0.1% NH₃·H₂O EtOH) as mobile phase, Flow Rate (mL/min): 70) to give:

### Example 103: the first eluting diastereoisomer, peak 1 (75 mg, 50% yield) as a yellow solid. LCMS m/z=415.2 (M+H)+¹H NMR (400 MHz, MeOH-d₄) δ=8.44 (s, 1H), 8.07 (s, 1H), 7.96-7.91 (m, 2H), 6.79 (s, 1H), 6.76-6.64 (m, 1H), 6.24-6.11 (m, 1H), 5.88-5.71 (m, 1H), 4.62-4.26 (m, 1H), 3.95 (s, 3H), 3.07-2.95 (m, 3H), 2.80-2.61 (m, 4H), 2.12-2.06 (m, 1H), 0.64-0.61 (m, 4H)

### Example 104: and the second eluting diastereoisomer, peak 2 (7.4 mg, 4.5% yield) as a yellow solid. LCMS m/z=415.2 (M+H)+ ¹H NMR (400 MHz, MeOH-d₄) δ=8.45 (s, 1H), 8.06 (s, 1H), 7.94 (s, 2H), 6.83 (s, 1H), 6.68 (s, 1H), 6.22-6.07 (m, 1H), 5.77-5.60 (m, 1H), 4.85-4.47 (m, 1H), 3.95 (s, 3H), 3.10-3.00 (m, 3H), 2.89 (s, 2H), 2.53-2.40 (m, 2H), 2.19-2.13 (m, 1H), 0.63-0.56 (m, 4H)

The absolute stereochemistry of the product in each peak was not assigned.

### Example 105. N-((1R,2R,3S)-2,3-dimethyl-3-((6-(1-methyl-1H-pyrazol-4-yl)pyrazolo[1,5-a]pyrazin-4-yl)oxy)cyclobutyl)-N-methylacrylamide

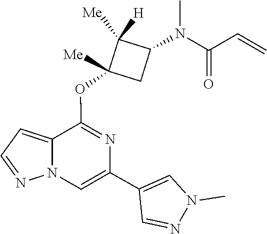 [SMILES: C=CC(=O)N(C)[C@@H]1C[C@](C)(Oc2nc(-c3cnn(C)c3)cn3nccc23)[C@@H]1C]

### 1. Synthesis of 3-(benzyloxy)-N,2-dimethylcyclobutan-1-amine

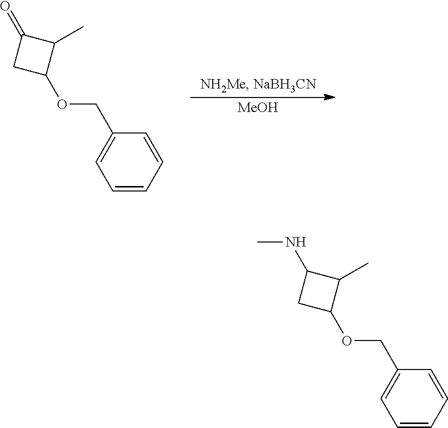

DIPEA (2.04 g, 15.8 mmol) was added to a solution of 3-(benzyloxy)-2-methylcyclobutan-1-one (1.5 g, 7.88 mmol) and NH₂Me (3.01 g, 31.5 mmol) in MeOH (80 mL) and the reaction mixture was stirred at 15° C. for 1 h. NaBH₃CN (991 mg, 15.8 mmol) was added and the reaction stirred at 15° C. for 2 h. The reaction mixture was concentrated in vacuo to give 3-(benzyloxy)-N,2-dimethylcyclobutan-1-amine (1.1 g, crude) as a white solid which was used for the next step without further purification. LCMS m/z=206.3 (M+H)+

### 2. Synthesis of tert-butyl (3-(benzyloxy)-2-methylcyclobutyl)(methyl)carbamate

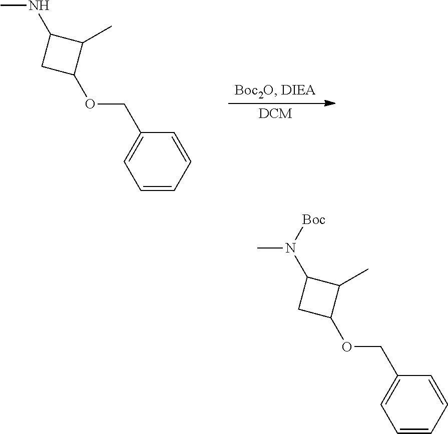

Boc₂O (1.75 g, 8.04 mmol) was added to a solution of 3-(benzyloxy)-N,2-dimethylcyclobutan-1-amine (1.1 g, 5.36 mmol) and DIPEA (1.38 g, 10.7 mmol) in DCM (100 mL) and the reaction was stirred at 10° C. for 2 h. The reaction mixture was concentrated in vacuum and the crude product was purified by silica gel chromatography (PE/EtOAc=15/1 to 8/1) to give tert-butyl (3-(benzyloxy)-2-methylcyclobutyl)(methyl)carbamate (1.3 g, 79% yield) as colorless oil. LCMS m/z=306.3 (M+H)+

### 3. Synthesis of tert-butyl (3-hydroxy-2-methylcyclobutyl)(methyl)carbamate

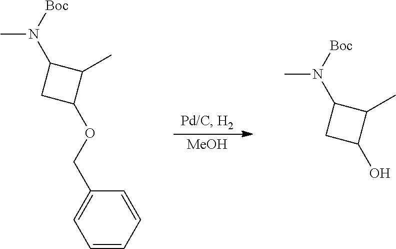

Pd/C (433 mg, 4.07 mmol) was added to a solution of tert-butyl (3-(benzyloxy)-2-methylcyclobutyl)(methyl)carbamate (1.3 g, 4.26 mmol) in MeOH (20 mL) and the reaction was stirred at 50° C. under H₂ (50 Psi) for 24 h. The mixture was filtered and concentrated under vacuum. The crude product was purified by silica gel (PE: EtOAc=1:0 to 1:1) to give tert-butyl (3-hydroxy-2-methylcyclobutyl)(methyl)carbamate (400 mg, 37% yield) as a colorless oil. LCMS m/z=216.3 (M+H)+.

### 4. Synthesis of tert-butyl methyl(2-methyl-3-oxocyclobutyl)carbamate

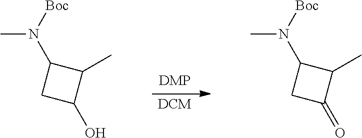

A mixture of tert-butyl (3-hydroxy-2-methylcyclobutyl)(methyl)carbamate (400 mg, 1.86 mmol) and DMP (541 mg, 2.79 mmol) in DCM (10 mL) was stirred at 20° C. for 1 h. Sat. Na₂SO₃ aq. (15 mL) was added and the mixture was extracted with DCM (3×40 mL). The combined organics were washed with brine (50 mL), dried over Na₂SO₄, filtered, and the filtrate concentrated in vacuo. The crude product was purified by silica gel column chromatography (PE:EtOAc=1:0 to 1:1) to give tert-butyl methyl(2-methyl-3-oxocyclobutyl)carbamate (250 mg, 50% yield) as colorless oil. LCMS m/z=214.3 (M+H)+

### 5. Synthesis of tert-butyl (3-hydroxy-2,3-dimethylcyclobutyl)(methyl)carbamate

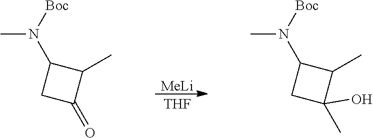

Methyllithium (1.6 M, 4 mL) was added to a solution of tert-butyl methyl(2-methyl-3-oxocyclobutyl)carbamate (250 mg, 1.17 mmol) in THF (5 mL) at −70° C. and the reaction was stirred at −70° C. for 1 h. Sat aq. NH₄Cl (10 mL) was added to quench the reaction, the mixture was filtered and the filtrate concentrated under vacuum to give tert-butyl (3-hydroxy-2,3-dimethylcyclobutyl)(methyl)carbamate (220 mg, crude) as yellow oil. LCMS m/z=230.3 (M+H)+

### 6. Synthesis of tert-butyl (3-((6-chloropyrazolo[1,5-a]pyrazin-4-yl)oxy)-2,3-dimethylcyclobutyl)(methyl)carbamate

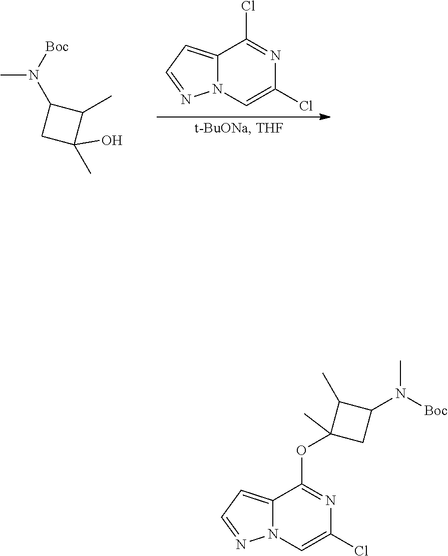

To a solution of tert-butyl (3-hydroxy-2,3-dimethylcyclobutyl)(methyl)carbamate (220 mg, 959 μmol) in THF (5 mL) was added t-BuONa (461 mg, 4.80 mmol) at 20° C. and the reaction was stirred for 1 h, then cooled to 0° C. 4,6-Dichloropyrazolo[1,5-a]pyrazine (330 mg, 1.76 mmol) was added and the reaction was stirred at 0° C. for 1 h. The reaction mixture was concentrated in vacuo and the crude was purified by prep HPLC (Column: Waters Xbridge BEH C18 100×25 mm×5 μm; Condition: water (0.225% FA)-MeCN, Begin B 50, End B 80, Gradient Time (min) 12, Flow Rate (mL/min) 25) to give tert-butyl (3-((6-chloropyrazolo[1,5-a]pyrazin-4-yl)oxy)-2,3-dimethylcyclobutyl)(methyl)carbamate (220 mg, 60% yield) as yellow oil. LCMS m/z=381.2 (M+H)+

### 7. Synthesis of tert-butyl (2,3-dimethyl-3-((6-(1-methyl-1H-pyrazol-4-yl)pyrazolo[1,5-a]pyrazin-4-yl)oxy)cyclobutyl)(methyl)carbamate

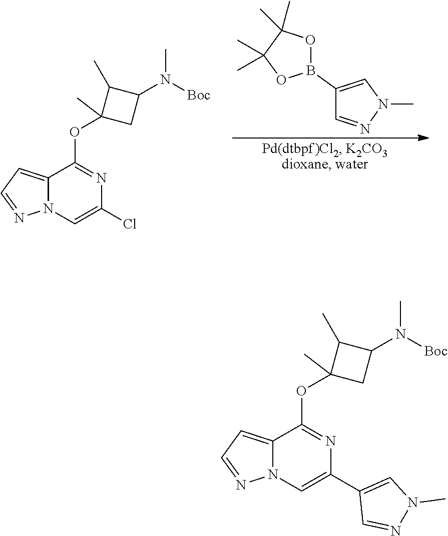

A mixture of tert-butyl (3-((6-chloropyrazolo[1,5-a]pyrazin-4-yl)oxy)-2,3-dimethylcyclobutyl)(methyl)carbamate (200 mg, 525 μmol), 1-methyl-4-(4,4,5,5-tetramethyl-1,3,2-dioxaborolan-2-yl)-1H-pyrazole (200 mg, 961 μmol), K₂CO₃ (218 mg, 1.58 mmol) and Pd(dtbpf)Cl₂ (34 mg, 53 μmol) in dioxane (2 mL)/water (0.4 mL) was purged with N₂ for 1 min and the reaction stirred at 90° C. for 2 h. The reaction mixture was concentrated in vacuo and the crude product was purified by prep HPLC (Column: Phenomenex Synergi C18 150×30 mm×4 μm; Condition: water (0.225% FA)-MeCN, Begin B 52, End B 82, Gradient Time (min) 11.5, Flow Rate (mL/min) 25) to give tert-butyl (2,3-dimethyl-3-((6-(1-methyl-1H-pyrazol-4-yl)pyrazolo[1,5-a]pyrazin-4-yl)oxy)cyclobutyl)(methyl)carbamate (130 mg, 58% yield.) as a yellow solid. LCMS m/z=427.3 (M+H)+

### 8. Synthesis of tert-butyl ((1S,2S,3S)-2,3-dimethyl-3-((6-(1-methyl-1H-pyrazol-4-yl)pyrazolo[1,5-a]pyrazin-4-yl)oxy)cyclobutyl)(methyl)carbamate, tert-butyl ((1R,2R,3R)-2,3-dimethyl-3-((6-(1-methyl-1H-pyrazol-4-yl)pyrazolo[1,5-a]pyrazin-4-yl)oxy)cyclobutyl)(methyl)carbamate, tert-butyl ((1S,2R,3S)-2,3-dimethyl-3-((6-(1-methyl-1H-pyrazol-4-yl)pyrazolo[1,5-a]pyrazin-4-yl)oxy)cyclobutyl)(methyl)carbamate, tert-butyl ((1R,2S,3R)-2,3-dimethyl-3-((6-(1-methyl-1H-pyrazol-4-yl)pyrazolo[1,5-a]pyrazin-4-yl)oxy)cyclobutyl)(methyl)carbamate, tert-butyl ((1S,2S,3R)-2,3-dimethyl-3-((6-(1-methyl-1H-pyrazol-4-yl)pyrazolo[1,5-a]pyrazin-4-yl)oxy)cyclobutyl)(methyl)carbamate and tert-butyl ((1R,2R,3S)-2,3-dimethyl-3-((6-(1-methyl-1H-pyrazol-4-yl)pyrazolo[1,5-a]pyrazin-4-yl)oxy)cyclobutyl)(methyl)carbamate

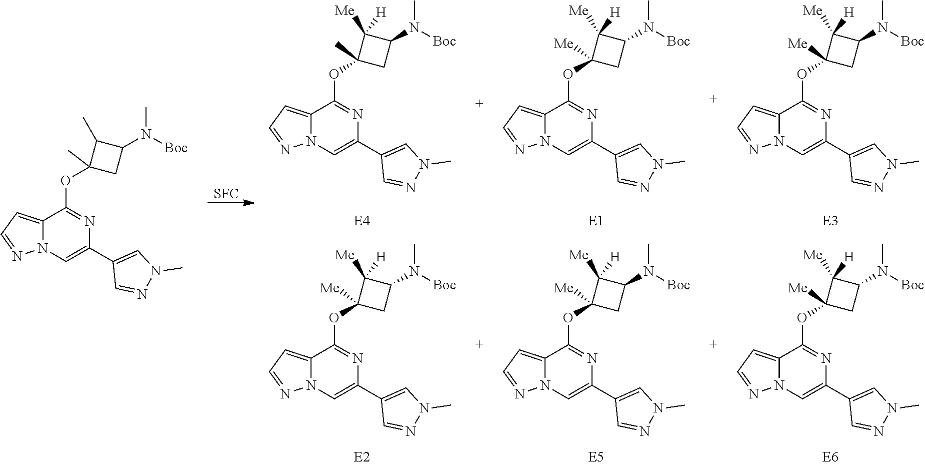

### Stereochemistry Arbitrarily Assigned

tert-Butyl (2,3-dimethyl-3-((6-(1-methyl-1H-pyrazol-4-yl)pyrazolo[1,5-a]pyrazin-4-yl)oxy)cyclobutyl)(methyl)carbamate (450 mg, 1.06 mmol) was separated by SFC (Column: DAICEL CHIRALPAK AD (250 mm×30 mm, 10 μm), 25% (0.1% NH₃H₂O/IPA), Flow Rate (mL/min) 60) to give:
the first eluting peak, E1 as a mixture with the fourth eluting peak, E4 (75 mg, crude) as a white solid, Rf=3.14 min and 3.224 min
the second eluting peak, E2, (40 mg, 9% yield) as a white solid, Rf=3.175 min
the third eluting peak, E3 as a mixture with the fifth eluting peak, E5 (140 mg, crude) as a white solid Rf=3.215 min and 3.302 min
the fourth eluting peak, E4 (60 mg, 13.33% yield) as a white solid, Rf=3.222 min
and the sixth eluting peak, E6 (50 mg, 11.1%.) as a white solid. Rf=3.739 min

### 9. Synthesis of (1R,2R,3S)—N,2,3-trimethyl-3-((6-(1-methyl-1H-pyrazol-4-yl)pyrazolo[1,5-a]pyrazin-4-yl)oxy)cyclobutan-1-amine

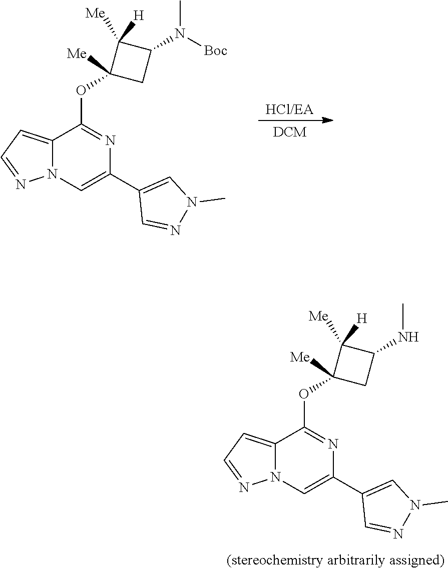

A solution of E6 (45 mg, 0.106 mmol) in DCM (5 mL) and HCl/EtOAc (4 M, 10 mL) was stirred at 15° C. for 1 h. The reaction mixture was concentrated in vacuo to give to give (1R,2R,3S)—N,2,3-trimethyl-3-((6-(1-methyl-1H-pyrazol-4-yl)pyrazolo[1,5-a]pyrazin-4-yl)oxy)cyclobutan-1-amine (35 mg, crude) as a yellow solid. LCMS m/z=327.3 (M+H)+.

### 10. Synthesis of N-((1R,2R,3S)-2,3-dimethyl-3-((6-(1-methyl-1H-pyrazol-4-yl)pyrazolo[1,5-a]pyrazin-4-yl)oxy)cyclobutyl)-N-methylacrylamide

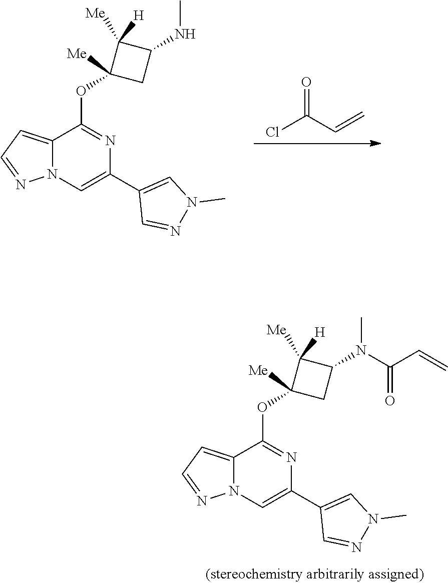

To a mixture of (1R,2R,3S)—N,2,3-trimethyl-3-((6-(1-methyl-1H-pyrazol-4-yl)pyrazolo[1,5-a]pyrazin-4-yl)oxy)cyclobutan-1-amine (45 mg, 0.138 mmol) and DIPEA (36 mg, 0.276 mmol) in DCM (20 mL) was added acryloyl chloride (12.5 mg, 0.138 mmol) at 0° C. and the reaction stirred for 5 min. MeOH (3 mL) was added dropwise to quench the reaction and the resulting mixture was stirred at 15° C. for 5 min. The solvent was removed in vacuo and the crude was purified by prep HPLC (Column: Welch Xtimate C18 150×25 mm×5 μm, Condition: water (10 mM NH₄HCO₃)-MeCN, Begin B 29, End B 59, Gradient Time (min) 10, Flow Rate (mL/min) 25) to give N-((1R,2R,3S)-2,3-dimethyl-3-((6-(1-methyl-1H-pyrazol-4-yl)pyrazolo[1,5-a]pyrazin-4-yl)oxy)cyclobutyl)-N-methylacrylamide (26 mg, 50% yield) as a pale yellow solid. LCMS m/z=381.3 (M+H)+ 1H NMR (500 MHz, MeOH-d₄) δ=8.46 (s, 1H), 8.09 (s, 1H), 8.02-7.88 (m, 2H), 6.80-6.64 (m, 2H), 6.33-6.20 (m, 1H), 5.84-5.73 (m, 1H), 4.45-4.30 (m, 1H), 4.01-3.93 (m, 3H), 3.20-3.08 (m, 3H), 3.06-2.82 (m, 3H), 1.93 (s, 3H), 1.11-1.00 (m, 3H).

### Example 106. N-((1S,2S,3S)-2,3-dimethyl-3-((6-(1-methyl-H-pyrazol-4-yl)pyrazolo[1,5-a]pyrazin-4-yl)oxy)cyclobutyl)-N-methylacrylamide

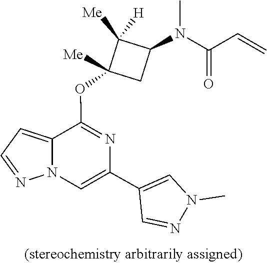 [SMILES: C=CC(=O)N(C)[C@H]1C[C@](C)(Oc2nc(-c3cnn(C)c3)cn3nccc23)[C@H]1C]

N-((1S,2S,3S)-2,3-Dimethyl-3-((6-(1-methyl-1H-pyrazol-4-yl)pyrazolo[1,5-a]pyrazin-4-yl)oxy)cyclobutyl)-N-methylacrylamide was obtained as a colorless oil, 11.3 mg, 28% yield from E4 (step 8, Example 102) following a similar procedure to that described in Example 105. The compound was purified by prep-HPLC (Column: Boston Prime C18 150×30 mm×5 m; Condition: water (0.05% NH₃H₂O+10 mM NH₄HCO₃)-MeCN; Begin B 38, End B 68, Gradient Time (min) 10, Flow Rate(mL/min) 25. LCMS m/z=403.2 (M+H)+ 1H NMR (500 MHz, MeOH-d₄) δ=8.46 (s, 1H), 8.07 (s, 1H), 7.96-7.95 (m, 2H), 6.84 (s, 1H), 6.78-6.70 (m, 1H), 6.22-6.12 (m, 1H), 5.76-5.67 (m, 1H), 4.74-4.36 (m, 1H), 3.96 (s, 3H), 3.13-3.05 (m, 4H), 2.78-2.70 (m, 1H), 2.58-2.42 (m, 1H), 1.89 (s, 3H), 1.35 (d, J=7.0 Hz, 3H).

### Example 107. N-((1R,2R,3R)-2,3-dimethyl-3-((6-(1-methyl-1H-pyrazol-4-yl)pyrazolo[1,5-a]pyrazin-4-yl)oxy)cyclobutyl)-N-methylacrylamide

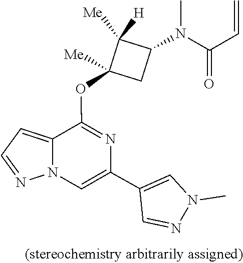 [SMILES: C=CC(=O)N(C)[C@@H]1C[C@@](C)(Oc2nc(-c3cnn(C)c3)cn3nccc23)[C@@H]1C]

N-((1R,2R,3R)-2,3-Dimethyl-3-((6-(1-methyl-1H-pyrazol-4-yl)pyrazolo[1,5-a]pyrazin-4-yl)oxy)cyclobutyl)-N-methylacrylamide was obtained as a yellow solid, 14 mg, 53% yield, from the mixture of E1 and E4 (step 8, Example 105) following the procedure described in Example 105. LCMS 381.3 (M+H)+. 1H NMR (500 MHz, MeOH-d₄) δ=8.46 (s, 1H), 8.09 (s, 1H), 8.02-7.88 (m, 2H), 6.80-6.64 (m, 2H), 6.33-6.20 (m, 1H), 5.84-5.73 (m, 1H), 4.45-4.30 (m, 1H), 4.01-3.93 (m, 3H), 3.20-3.08 (m, 3H), 3.06-2.82 (m, 3H), 1.93 (s, 3H), 1.11-1.00 (m, 3H).

### Example 108. N-((1R,2S,3R)-2,3-dimethyl-3-((6-(1-methyl-1H-pyrazol-4-yl)pyrazolo[1,5-a]pyrazin-4-yl)oxy)cyclobutyl)-N-methylacrylamide

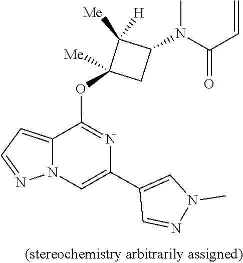 [SMILES: C=CC(=O)N(C)[C@@H]1C[C@@](C)(Oc2nc(-c3cnn(C)c3)cn3nccc23)[C@H]1C]

N-((1R,2S,3R)-2,3-dimethyl-3-((6-(1-methyl-1H-pyrazol-4-yl)pyrazolo[1,5-a]pyrazin-4-yl)oxy)cyclobutyl)-N-methylacrylamide was obtained as a white solid, 44 mg, 60% yield, from E2, Example 105, following the procedure described in Example 105. LCMS 381.3 (M+H)+. 1H NMR (500 MHz, MeOH-d₄) δ ppm=8.43 (s, 1H), 8.05 (s, 1H), 7.94-7.92 (m, 2H), 6.82-6.71 (m, 2H), 6.23-6.19 (m, 1H), 5.76-5.74 (m, 1H), 4.44-4.02 (m, 1H), 3.94 (s, 3H), 3.08-3.04 (m, 3H), 3.99 (s, 1H), 2.89-2.69 (m, 2H), 1.76 (s, 3H), 1.29 (d, J=7.0 Hz, 3H).

### Example 109. N-((1S,2R,3S)-2,3-dimethyl-3-((6-(1-methyl-1H-pyrazol-4-yl)pyrazolo[1,5-a]pyrazin-4-yl)oxy)cyclobutyl)-N-methylacrylamide

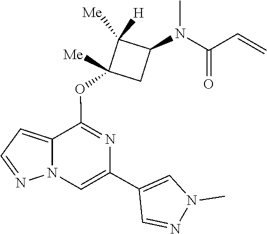 [SMILES: C=CC(=O)N(C)[C@H]1C[C@](C)(Oc2nc(-c3cnn(C)c3)cn3nccc23)[C@@H]1C]

### 1. Synthesis of tert-butyl ((1S,2R,3S)-2,3-dimethyl-3-((6-(1-methyl-1H-pyrazol-4-yl)pyrazolo[1,5-a]pyrazin-4-yl)oxy)cyclobutyl)(methyl)carbamate and tert-butyl ((1S,2S,3R)-2,3-dimethyl-3-((6-(1-methyl-1H-pyrazol-4-yl)pyrazolo[1,5-a]pyrazin-4-yl)oxy)cyclobutyl)(methyl)carbamate

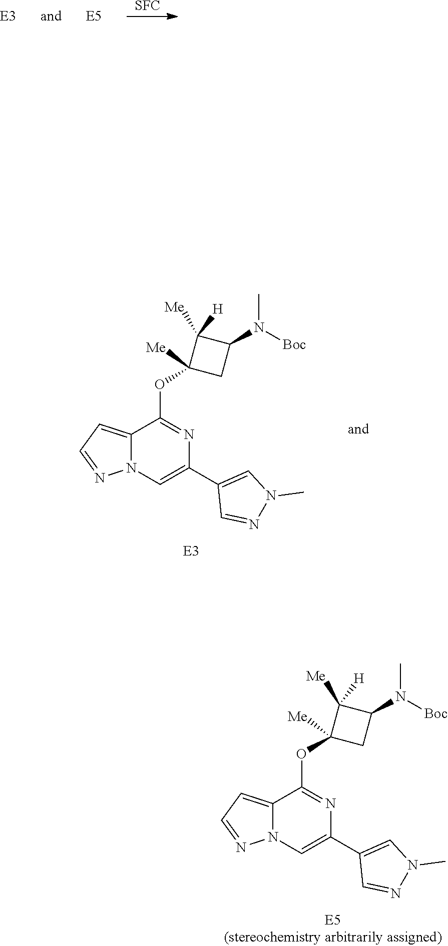 [SMILES: "SFC" ; C[C@@H]1[C@@H](N(C)C(=O)OC(C)(C)C)C[C@]1(C)Oc1nc(-c2cnn(C)c2)cn2nccc12 ; C[C@H]1[C@@H](N(C)C(=O)OC(C)(C)C)C[C@@]1(C)Oc1nc(-c2cnn(C)c2)cn2nccc12]

A mixture of E3 and E5 from step 8, Example 105 (140 mg, 0.328 mmol) was separated by SFC (Column: DAICEL CHIRALPAK AD (250 mm×30 mm, 10 um); Condition: 20% (0.1% NH₃H₂O EtOH), Gradient Time (min), Flow Rate (mL/min) 60) to give:

The first eluting diastereoisomer, E3, tert-butyl ((1S,2R,3S)-2,3-dimethyl-3-((6-(1-methyl-1H-pyrazol-4-yl)pyrazolo[1,5-a]pyrazin-4-yl)oxy)cyclobutyl)(methyl)carbamate (stereochemistry arbitrarily assigned)(61 mg, 44% yield) as a colorless oil. 1H NMR (500 MHz, MeOH-d₄) δ=8.33 (s, 1H), 7.98 (s, 1H), 7.90-7.80 (m, 2H), 6.72 (d, J=2.0 Hz, 1H), 3.92 (s, 4H), 2.85-2.71 (m, 5H), 2.60-2.48 (m, 1H), 1.69 (s, 3H), 1.46 (s, 9H), 1.24 (d, J=7.0 Hz, 3H).
and the second eluting diastereoisomer, E5, tert-butyl ((1S,2S,3R)-2,3-dimethyl-3-((6-(1-methyl-1H-pyrazol-4-yl)pyrazolo[1,5-a]pyrazin-4-yl)oxy)cyclobutyl)(methyl)carbamate (stereochemistry arbitrarily assigned)(45 mg, 32% yield) as a colorless oil. 1H NMR (500 MHz, MeOH-d₄) δ=8.45-8.34 (m, 1H), 8.09-7.98 (m, 1H), 7.93-7.86 (m, 2H), 6.74 (d, J=2.0 Hz, 1H), 4.11-3.89 (m, 4H), 3.05-2.69 (m, 6H), 1.91-1.80 (m, 3H), 1.48 (s, 9H), 1.04 (d, J=7.0 Hz, 3H).

### 2. Synthesis of (1S,2R,3S)—N,2,3-trimethyl-3-((6-(1-methyl-1H-pyrazol-4-yl)pyrazolo[1,5-a]pyrazin-4-yl)oxy)cyclobutan-1-amine hydrochloride

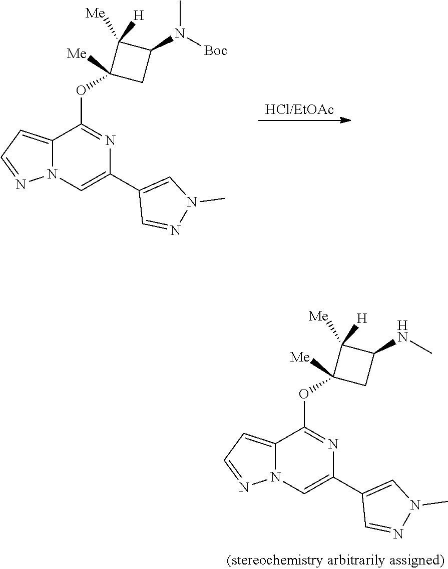 [SMILES: C[C@@H]1[C@@H](N(C)C(=O)OC(C)(C)C)C[C@]1(C)Oc1nc(-c2cnn(C)c2)cn2nccc12 ; "HCl/EtOAc" ; CN[C@H]1C[C@](C)(Oc2nc(-c3cnn(C)c3)cn3nccc23)[C@@H]1C]

HCl in EtOAc (4 M, 3 mL) was added to a solution of E3, tert-butyl ((1S,2R,3S)-2,3-dimethyl-3-((6-(1-methyl-1H-pyrazol-4-yl)pyrazolo[1,5-a]pyrazin-4-yl)oxy)cyclobutyl)(methyl)carbamate (61 mg, 143 μmol) in DCM (1 mL) and the reaction stirred at 25° C. for 1 h. The reaction mixture was concentrated to give (1S,2R,3S)—N,2,3-trimethyl-3-((6-(1-methyl-1H-pyrazol-4-yl)pyrazolo[1,5-a]pyrazin-4-yl)oxy)cyclobutan-1-amine hydrochloride (46 mg, 99% yield) as a yellow solid, which was used in the next step directly. LCMS m/z=327.2 (M+H)+

### 3. Synthesis of N—((1S,2R,3S)-2,3-dimethyl-3-((6-(1-methyl-1H-pyrazol-4-yl)pyrazolo[1,5-a]pyrazin-4-yl)oxy)cyclobutyl)-N-methylacrylamide

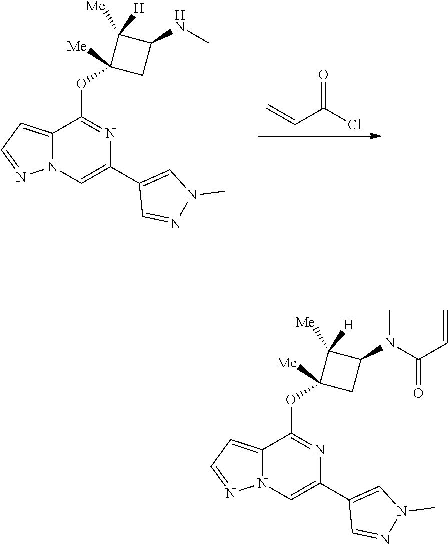

To the mixture of (1S,2R,3S)—N,2,3-trimethyl-3-((6-(1-methyl-1H-pyrazol-4-yl)pyrazolo[1,5-a]pyrazin-4-yl)oxy)cyclobutan-1-amine hydrochloride (46 mg, 125 μmol) and DIPEA (55 mg, 422 μmol) in DCM (3 mL) was added acryloyl chloride (13 mg, 140 μmol) at 0° C. and the reaction stirred for 5 min. MeOH (1 mL) was added dropwise and the resulting mixture was stirred at 25° C. for 10 min. The solvent was removed in vacuo and the crude product was purified by prep HPLC (Column: Boston Prime C18 150×30 mm×5 um; Condition: water (0.05% NH₃H₂O+10 mM NH₄HCO₃)-MeCN, Begin B 40, End B 70, Gradient Time (min) 10, Flow Rate (mL/min) 25) to give N-((1S,2R,3S)-2,3-dimethyl-3-((6-(1-methyl-1H-pyrazol-4-yl)pyrazolo[1,5-a]pyrazin-4-yl)oxy)cyclobutyl)-N-methylacrylamide (stereochemistry arbitrarily assigned) (32 mg, 60% yield) as a white solid. LCMS m/z=381.2 (M+H)+. 1H NMR (500 MHz, MeOH-d₄) δ=8.42 (s, 1H), 8.09-8.02 (m, 1H), 7.96-7.89 (m, 2H), 6.91-6.70 (m, 2H), 6.25-6.22 (m, 1H), 5.77 (t, J=8.5 Hz, 1H), 4.50-3.99 (m, 1H), 3.95 (s, 3H), 3.09-2.86 (m, 5H), 2.75-2.54 (m, 1H), 1.83-1.74 (m, 3H), 1.30 (d, J=6.5 Hz, 3H).

### Example 110. N-((1S,2S,3R)-2,3-dimethyl-3-((6-(1-methyl-1H-pyrazol-4-yl)pyrazolo[1,5-a]pyrazin-4-yl)oxy)cyclobutyl)-N-methylacrylamide

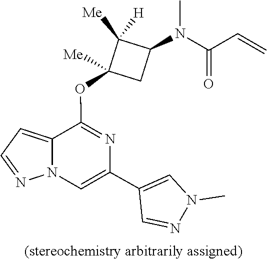 [SMILES: C=CC(=O)N(C)[C@H]1C[C@@](C)(Oc2nc(-c3cnn(C)c3)cn3nccc23)[C@H]1C]

N-((1S,2S,3R)-2,3-dimethyl-3-((6-(1-methyl-1H-pyrazol-4-yl)pyrazolo[1,5-a]pyrazin-4-yl)oxy)cyclobutyl)-N-methylacrylamide was obtained as a white solid, 23 mg, 59% yield, from E3, tert-butyl ((1S,2S,3R)-2,3-dimethyl-3-((6-(1-methyl-1H-pyrazol-4-yl)pyrazolo[1,5-a]pyrazin-4-yl)oxy)cyclobutyl)(methyl)carbamate (step 1, Example 109), following the method described in Example 109. LCMS m/z=381.2 (M+H)+. 1H NMR (500 MHz, MeOH-d₄) δ=8.46 (s, 1H), 8.08 (s, 1H), 7.97-7.88 (m, 2H), 6.85-6.62 (m, 2H), 6.37-6.14 (m, 1H), 5.85-5.70 (m, 1H), 4.49-4.27 (m, 1H), 4.04-3.92 (m, 3H), 3.1-2.81 (m, 6H), 1.93 (s, 3H), 1.11-0.98 (m, 3H).

### Example 111 & 112. 1-((1S,6S,7R)-7-methyl-7-((6-(1-methyl-1H-pyrazol-4-yl)pyrazolo[1,5-a]pyrazin-4-yl)oxy)-2-azabicyclo[4.2.0]octan-2-yl)prop-2-en-1-one and 1-((1R,6R,7S)-7-methyl-7-((6-(1-methyl-1H-pyrazol-4-yl)pyrazolo[1,5-a]pyrazin-4-yl)oxy)-2-azabicyclo[4.2.0]octan-2-yl)prop-2-en-1-one

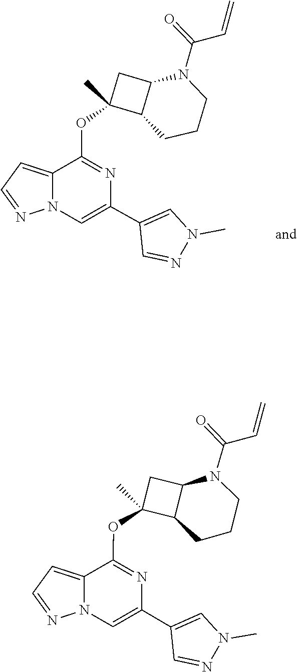 [SMILES: C=CC(=O)N1CCC[C@H]2[C@@H]1C[C@@]2(C)Oc1nc(-c2cnn(C)c2)cn2nccc12 ; C=CC(=O)N1CCC[C@@H]2[C@H]1C[C@]2(C)Oc1nc(-c2cnn(C)c2)cn2nccc12]

### 1. Synthesis of racemic benzyl (1R,6R)-7-hydroxy-7-methyl-2-azabicyclo[4.2.0]octane-2-carboxylate

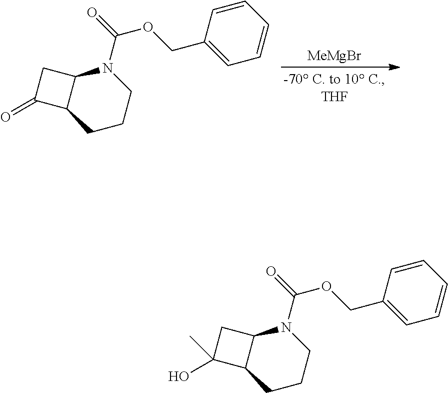 [SMILES: "MeMgBr" ; O=C1C[C@@H]2[C@H]1CCCN2C(=O)OCc1ccccc1 ; CC1(O)C[C@@H]2[C@H]1CCCN2C(=O)OCc1ccccc1]

A solution of racemic benzyl (1R,6R)-7-oxo-2-azabicyclo[4.2.0]octane-2-carboxylate (300 mg, 1.16 mmol) in THF (2 mL) was added to a solution of methylmagnesium bromide (193 mg, 1.62 mmol) in THF (10 mL)−78° C. under N₂ and the reaction stirred at 15° C. for 0.5 h. The mixture was filtered and concentrated under vacuum and the crude product was purified by column chromatography on silica gel (PE/EtOAc=1/0 to 1/3) to give racemic benzyl (1R,6R)-7-hydroxy-7-methyl-2-azabicyclo[4.2.0]octane-2-carboxylate (250 mg, 48% yield) as yellow oil. LCMS m/z=276.1 (M+H)+

### 2. Synthesis of racemic benzyl (1R,6R)-7-((6-chloropyrazolo[1,5-a]pyrazin-4-yl)oxy)-7-methyl-2-azabicyclo[4.2.0]octane-2-carboxylate

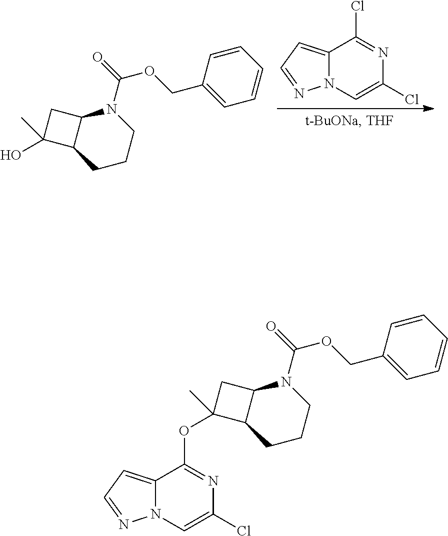

To a solution of racemic benzyl (1R,6R)-7-hydroxy-7-methyl-2-azabicyclo[4.2.0]octane-2-carboxylate (250 mg, 908 μmol) in THF (4 mL) was added t-BuONa (262 mg, 2.72 mmol) at 0° C. and the solution stirred for 5 mins. 4,6-Dichloropyrazolo[1,5-a]pyrazine (239 mg, 1.27 mmol) was added and the reaction stirred at 0° C. for 0.5 h. The mixture was filtered, concentrated under vacuum and the crude product was purified by column chromatography on silica gel (PE/EtOAc=1/0 to 1/1) to give racemic benzyl (1R,6R)-7-((6-chloropyrazolo[1,5-a]pyrazin-4-yl)oxy)-7-methyl-2-azabicyclo[4.2.0]octane-2-carboxylate (200 mg, 46% yield) as yellow oil. LCMS m/z=427.2 (M+H)+

### 3. Synthesis of racemic benzyl (1R,6R)-7-methyl-7-((6-(1-methyl-1H-pyrazol-4-yl)pyrazolo[1,5-a]pyrazin-4-yl)oxy)-2-azabicyclo[4.2.0]octane-2-carboxylate

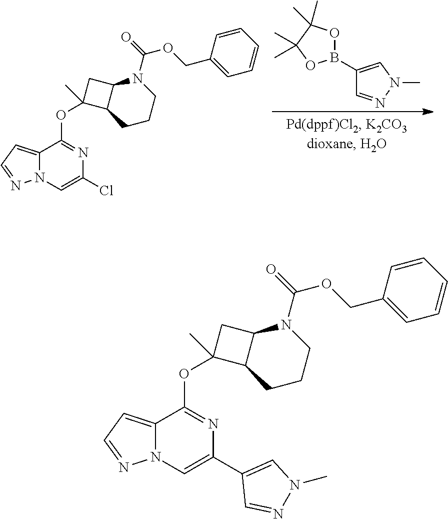

To a solution of racemic benzyl (1R,6R)-7-((6-chloropyrazolo[1,5-a]pyrazin-4-yl)oxy)-7-methyl-2-azabicyclo[4.2.0]octane-2-carboxylate (150 mg, 351 μmol) in dioxane (2 mL) and water (0.3 mL) was added 1-methyl-4-(4,4,5,5-tetramethyl-1,3,2-dioxaborolan-2-yl)-1H-pyrazole (219 mg, 1.05 mmol), K₂CO₃ (97 mg, 703 μmol) and Pd(dppf)Cl₂ (34 mg, 53 μmol) and the reaction was stirred at 80° C. for 4 h under N₂. The mixture was filtered and concentrated under vacuum. The crude product was purified by column chromatography on silica gel (PE/EtOAc=1/0 to 0/1) to give of racemic benzyl (1R,6R)-7-methyl-7-((6-(1-methyl-1H-pyrazol-4-yl)pyrazolo[1,5-a]pyrazin-4-yl)oxy)-2-azabicyclo[4.2.0]octane-2-carboxylate (40 mg, 58% yield) as yellow oil. LCMS m/z=473.2 (M+H)+

### 4. Synthesis of racemic 6-(1-methyl-1H-pyrazol-4-yl)-4-(((1R,6R)-7-methyl-2-azabicyclo[4.2.0]octan-7-yl)oxy)pyrazolo[1,5-a]pyrazine

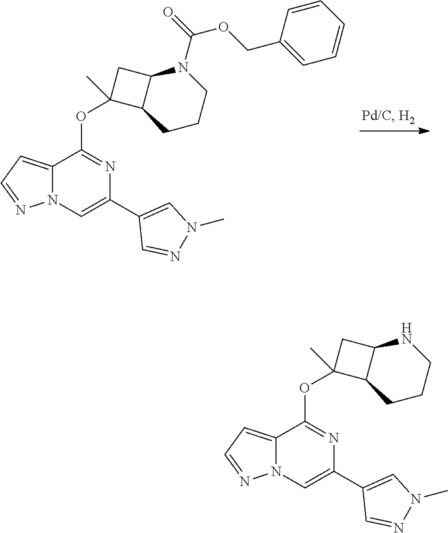 [SMILES: Cn1cc(-c2cn3nccc3c(OC3(C)C[C@@H]4[C@H]3CCCN4C(=O)OCc3ccccc3)n2)cn1 ; Cn1cc(-c2cn3nccc3c(OC3(C)C[C@H]4NCCC[C@H]43)n2)cn1]

To a solution of racemic benzyl (1R,6R)-7-methyl-7-((6-(1-methyl-1H-pyrazol-4-yl)pyrazolo[1,5-a]pyrazin-4-yl)oxy)-2-azabicyclo[4.2.0]octane-2-carboxylate (220 mg, 466 μmol) in DMF (10 mL) was added Pd/C (198 mg, 1.86 mmol) and the mixture was stirred at 15° C. for 3 h under H₂. The resulting solid was collected by vacuum filtration, washed with DCM (2×10 mL) and dried under high vacuum to give racemic 6-(1-methyl-1H-pyrazol-4-yl)-4-(((1R,6R)-7-methyl-2-azabicyclo[4.2.0]octan-7-yl)oxy)pyrazolo[1,5-a]pyrazine (150 mg, crude) as white solid. LCMS m/z=339.1 (M+H)+

### 5. Synthesis of racemic 1-((1R,6R)-7-methyl-7-((6-(1-methyl-1H-pyrazol-4-yl)pyrazolo[1,5-a]pyrazin-4-yl)oxy)-2-azabicyclo[4.2.0]octan-2-yl)prop-2-en-1-one

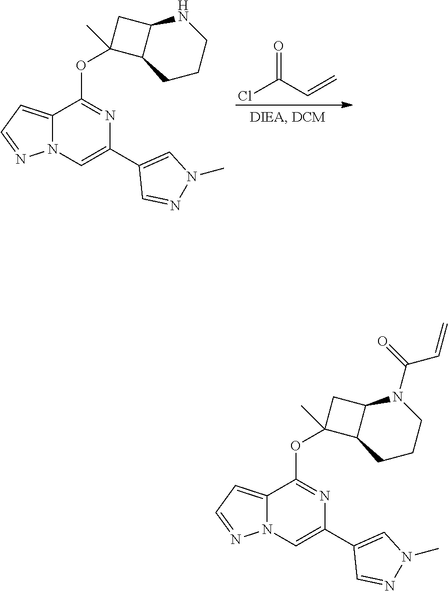

To a solution of racemic 6-(1-methyl-1H-pyrazol-4-yl)-4-(((1R,6R)-7-methyl-2-azabicyclo[4.2.0]octan-7-yl)oxy)pyrazolo[1,5-a]pyrazine (150 mg, 443 μmol) in DCM (10 mL) was added DIPEA (115 mg, 887 μmol) at 15° C. Acryloyl chloride (80 mg, 887 μmol) was added into the mixture at 0° C. and the reaction stirred at 0° C. for 0.5 h. The mixture was filtered and concentrated under vacuum. The crude was purified by column chromatography on silica gel (PE/EtOAc=1/0 to 0/1) to give racemic 1-((1R,6R)-7-methyl-7-((6-(1-methyl-1H-pyrazol-4-yl)pyrazolo[1,5-a]pyrazin-4-yl)oxy)-2-azabicyclo[4.2.0]octan-2-yl)prop-2-en-1-one (130 mg, 75% yield) as yellow oil. LCMS m/z=393.1 (M+H)+

### 6. Synthesis of 1-((1S,6S,7R)-7-methyl-7-((6-(1-methyl-1H-pyrazol-4-yl)pyrazolo[1,5-a]pyrazin-4-yl)oxy)-2-azabicyclo[4.2.0]octan-2-yl)prop-2-en-1-one and 1-((1R,6R,7S)-7-methyl-7-((6-(1-methyl-1H-pyrazol-4-yl)pyrazolo[1,5-a]pyrazin-4-yl)oxy)-2-azabicyclo[4.2.0]octan-2-yl)prop-2-en-1-one

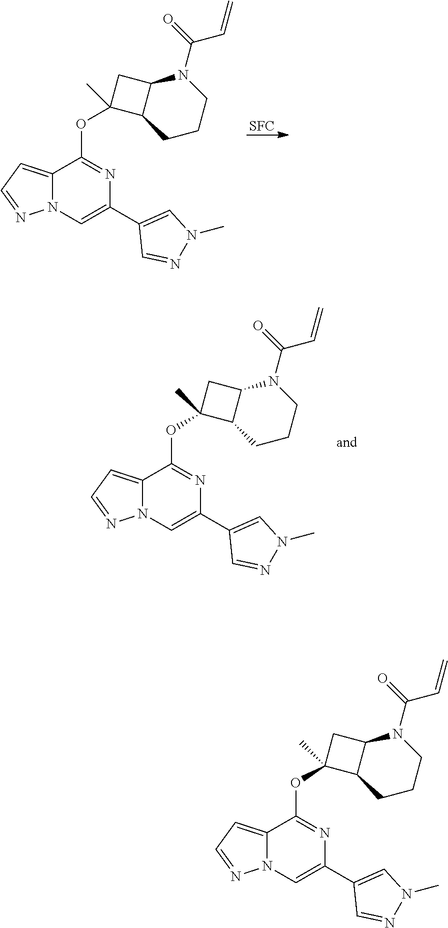

Racemic 1-((1R,6R)-7-methyl-7-((6-(1-methyl-1H-pyrazol-4-yl)pyrazolo[1,5-a]pyrazin-4-yl)oxy)-2-azabicyclo[4.2.0]octan-2-yl)prop-2-en-1-one (150 mg, 382 μmol) was purified by SFC (column: DAICEL CHIRALPAK AD(250 mm*30 mm, 10 um), 50% [0.1% NH₃H₂O, EtOH] as mobile phase, Flow Rate (mL/min): 80) to give:

### Example 111: The first eluting enantiomer, Peak 1 (51.7 mg, 34% yield) as white solid. LCMS m/z=415.1 (M+Na)+. 1H NMR (500 MHz, MeOH-d₄) δ=8.44 (d, J=2.0 Hz, 1H), 8.05 (s, 1H), 7.93-7.92 (m, 2H), 6.84-6.78 (m, 2H), 6.26-6.17 (m, 1H), 5.79-5.73 (m, 1H), 4.69-4.42 (m, 1H), 4.19 (d, J=13.0 Hz, 0.5H), 3.95 (s, 3H), 3.93-3.87 (m, 0.5H), 3.31-3.29 (m, 1H), 3.11-2.98 (m, 1H), 2.79-2.65 (m, 2H), 1.95-1.92 (m, 1H), 1.91-1.89 (m, 3H), 1.85-1.79 (m, 2H), 1.40-1.34 (m, 1H)

### Example 112: the second eluting enantiomer, Peak 2 (51.2 mg, 34% yield) as white solid. LCMS m/z=393.1 (M+H)+. 1H NMR (500 MHz, MeOH-d₄) δ=8.44 (d, J=2.0 Hz, 1H), 8.05 (s, 1H), 7.93-7.92 (m, 2H), 6.84-6.78 (m, 2H), 6.26-6.18 (m, 1H), 5.79-5.72 (m, 1H), 4.69-4.42 (m, 1H), 4.19 (d, J=13.0 Hz, 0.5H), 3.95 (s, 3H), 3.93-3.87 (m, 0.5H), 3.28-3.27 (m, 1H), 3.09-3.07 (m, 1H), 2.80-2.74 (m, 2H), 1.95-1.92 (m, 1H), 1.91-1.89 (m, 3H), 1.85-1.79 (m, 2H), 1.40-1.37 (m, 1H)

The absolute stereochemistry of the product in each peak was not assigned.

### Examples 113, 114, 115 & 116. N-methyl-N-((1S,3S,6R)-1-((6-(1-methyl-1H-pyrazol-4-yl)pyrazolo[1,5-a]pyrazin-4-yl)oxy)bicyclo[4.1.0]heptan-3-yl)but-2-ynamide, N-methyl-N—((1S,3R,6R)-1-((6-(1-methyl-1H-pyrazol-4-yl)pyrazolo[1,5-a]pyrazin-4-yl)oxy)bicyclo[4.1.0]heptan-3-yl)but-2-ynamide, N-methyl-N-((1R,3R,6S)-1-((6-(1-methyl-1H-pyrazol-4-yl)pyrazolo[1,5-a]pyrazin-4-yl)oxy)bicyclo[4.1.0]heptan-3-yl)but-2-ynamide and N-methyl-N-((1R,3S,6S)-1-((6-(1-methyl-1H-pyrazol-4-yl)pyrazolo[1,5-a]pyrazin-4-yl)oxy)bicyclo[4.1.0]heptan-3-yl)but-2-ynamide

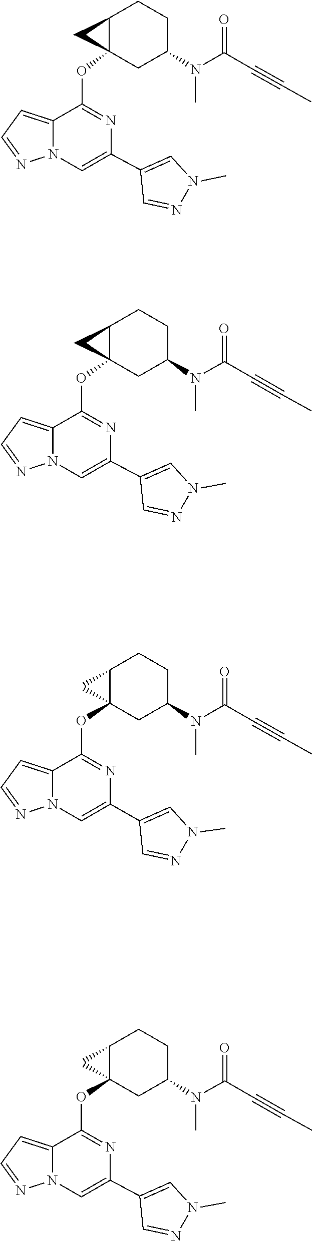 [SMILES: CC#CC(=O)N(C)[C@H]1CC[C@@H]2C[C@]2(Oc2nc(-c3cnn(C)c3)cn3nccc23)C1 ; CC#CC(=O)N(C)[C@@H]1CC[C@@H]2C[C@]2(Oc2nc(-c3cnn(C)c3)cn3nccc23)C1 ; CC#CC(=O)N(C)[C@@H]1CC[C@H]2C[C@@]2(Oc2nc(-c3cnn(C)c3)cn3nccc23)C1 ; CC#CC(=O)N(C)[C@H]1CC[C@H]2C[C@@]2(Oc2nc(-c3cnn(C)c3)cn3nccc23)C1]

### 1. Synthesis of 3-Hydroxyhept-6-enenitrile

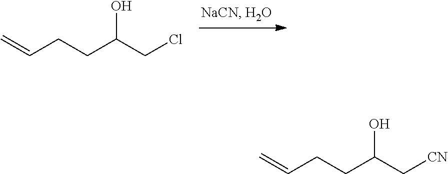

To a solution of NaCN (39.7 g, 809 mmol) in H₂O (350 mL) was added 1-chlorohex-5-en-2-ol (72 g, 535 mmol) dropwise at 35° C. and the reaction was stirred at 60° C. for 24 h under N₂ atmosphere. Water (100 mL) was added, the mixture extracted with EtOAc (200 mL×3), the combined organic layers were washed with brine (500 mL), dried over Na₂SO₄, filtered and concentrated under reduced pressure. The residue was purified by silica gel column chromatography (PE/EtOAc=15/1 to 5/1) to give 3-hydroxyhept-6-enenitrile (66 g, 99% yield) as yellow oil. ¹H NMR (400 MHz, CDCl₃) δ ppm 5.82-5.75 (m, 1H), 5.10-4.96 (m, 2H), 3.94 (br s, 1H), 2.78 (br s, 1H), 2.60-2.44 (m, 2H), 2.26-2.09 (m, 2H), 1.72-1.61 (m, 2H).

### 2. Synthesis of methyl 3-hydroxyhept-6-enoate

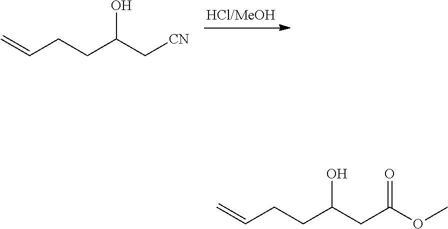

3-Hydroxyhept-6-enenitrile (66 g, 527 mmol) was dissolved in HCl/MeOH (4 M, 700 mL) at 20° C. and the reaction stirred at 90° C. for 12 h under N₂. The mixture was concentrated under reduced pressure and the crude was purified on silica gel by column chromatography (PE/EtOAc=15/1 to 5/1) to give methyl 3-hydroxyhept-6-enoate (40 g, 48% yield) as yellow oil. 1H NMR (400 MHz, CDCl₃) δ ppm 5.83-5.75 (m, 1H), 5.11-4.91 (m, 2H), 4.02-3.97 (m, 1H), 3.68 (s, 3H), 2.98 (d, J=4.0 Hz, 1H), 2.53-2.36 (m, 2H), 2.26-2.08 (m, 2H), 1.67-1.48 (m, 2H).

### 3. Synthesis of methyl 3-((tert-butyldimethylsilyl)oxy)hept-6-enoate

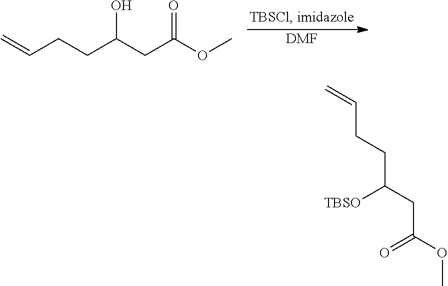

To a solution of methyl 3-hydroxyhept-6-enoate (40 g, 253 mmol) in DMF (400 mL) was added imidazole (34.4 g, 506 mmol) and TBSCl (45.7 g, 303 mmol) at 0° C. and the reaction was stirred at 20° C. for 12 h under N₂ atmosphere. Water (200 mL) was added, the mixture extracted with EtOAc (200 mL×3), the combined organic layers were washed with H₂O (200 mL×3) and brine (300 mL), dried over Na₂SO₄, filtered and concentrated under reduced pressure. The crude was purified on silica gel by column chromatography (PE/EtOAc=15/1 to 5/1) to give methyl 3-((tert-butyldimethylsilyl)oxy)hept-6-enoate (51 g, 74% yield) as yellow oil. 1H NMR (400 MHz, CDCl₃) δ ppm 5.84-5.77 (m, 1H), 5.06-4.92 (m, 2H), 4.19-4.07 (m, 1H), 3.66 (s, 3H), 2.52-2.38 (m, 2H), 2.15-2.05 (m, 2H), 1.62-1.57 (m, 2H), 0.87-0.86 (m, 9H), 0.05 (d, J=12.0 Hz, 6H).

### 4. Synthesis of 3-((tert-butyldimethylsilyl)oxy)bicyclo[4.1.0]heptan-1-ol

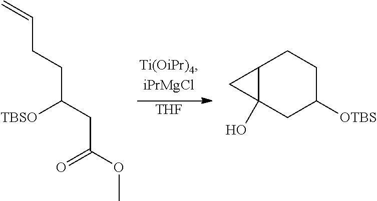 [SMILES: C=CCCC(CC(=O)OC)O[Si](C)(C)C(C)(C)C ; CC(C)(C)[Si](C)(C)OC1CCC2CC2(O)C1 ; "THF"]

To a solution of methyl 3-((tert-butyldimethylsilyl)oxy)hept-6-enoate (51.0 g, 187 mmol) in THF (500 mL) was added Ti(OiPr)₄ (106.4 g, 374 mmol) at 20° C., and the solution cooled to −75° C. i-PrMgCl (2 M, 374.4 mL) was added dropwise under N₂ atmosphere and once addition was complete the reaction was stirred at 20° C. for 2 h. The reaction was slowly quenched with sat.aq NH₄Cl solution (200 mL) at 0° C. and the mixture was extracted with EtOAc (200 mL×3). The combined organic layers were washed with brine (500 mL), dried over Na₂SO₄, filtered and concentrated under reduced pressure. The crude was purified by silica gel column chromatography (PE/EtOAc=15/1 to 5/1) to give 3-((tert-butyldimethylsilyl)oxy)bicyclo[4.1.0]heptan-1-ol (25 g, 55% yield) as yellow oil. LCMS m/z=243.2 (M+H)+

### 5. Synthesis of 4-((3-((tert-butyldimethylsilyl)oxy)bicyclo[4.1.0]heptan-1-yl)oxy)-6-chloropyrazolo[1,5-a]pyrazine

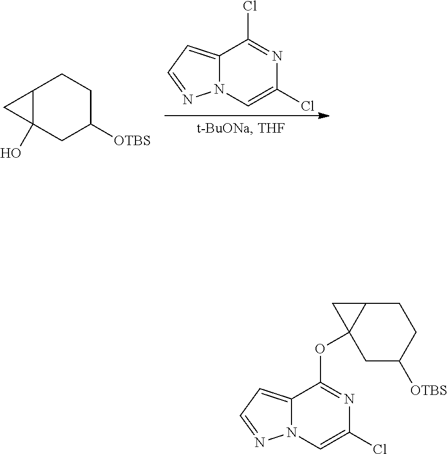 [SMILES: CC(C)(C)[Si](C)(C)OC1CCC2CC2(O)C1 ; Clc1cn2nccc2c(Cl)n1 ; CC(C)(C)[Si](C)(C)OC1CCC2CC2(Oc2nc(Cl)cn3nccc23)C1]

To a solution of 3-((tert-butyldimethylsilyl)oxy)bicyclo[4.1.0]heptan-1-ol (2.50 g, 10.3 mmol) in THF (200 mL) was added t-BuONa (2.97 g, 30.9 mmol) and the solution cooled to 0° C. 4,6-Dichloropyrazolo[1,5-a]pyrazine (1.94 g, 10.3 mmol) was added and the reaction was stirred at 0° C. for 12 h. The mixture was concentrated under vacuum and the crude was purified by column chromatography on silica gel (PE/EtOAc=1/0 to 8/1) to give 4-((3-((tert-butyldimethylsilyl)oxy)bicyclo[4.1.0]heptan-1-yl)oxy)-6-chloropyrazolo[1,5-a]pyrazine (2.5 g, 47% yield) as colorless oil. LCMS m/z=394.2 (M+H)+

### 6. Synthesis of 4-((3-((tert-butyldimethylsilyl)oxy)bicyclo[4.1.0]heptan-1-yl)oxy)-6-(1-methyl-1H-pyrazol-4-yl)pyrazolo[1,5-a]pyrazine

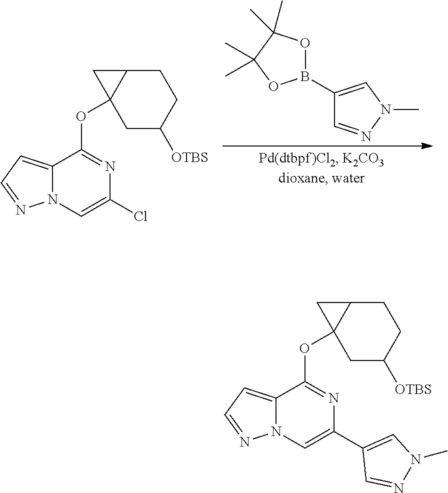

To a solution of 4-((3-((tert-butyldimethylsilyl)oxy)bicyclo[4.1.0]heptan-1-yl)oxy)-6-chloropyrazolo[1,5-a]pyrazine (2.3 g, 4.1 mmol) in dioxane (50 mL) and water (10 mL) was added K₂CO₃ (1.69 g, 12.3 mmol) and 1-methyl-4-(4,4,5,5-tetramethyl-1,3,2-dioxaborolan-2-yl)-1H-pyrazole (1.28 g, 6.13 mmol) at 20° C. Pd(dtbpf)Cl₂ (533 mg, 817 μmol) was added and the reaction was stirred at 90° C. under N₂ for 4 h. The mixture was concentrated under vacuum and the crude was purified by column chromatography on silica gel (PE/EtOAc=1/0 to 0/1) to give 4-((3-((tert-butyldimethylsilyl)oxy)bicyclo [4.1.0]heptan-1-yl)oxy)-6-(1-methyl-1H-pyrazol-4-yl)pyrazolo[1,5-a]pyrazine (1.7 g, 92% yield) as yellow oil. LCMS m/z=440.3 (M+H)+

### 7. Synthesis of 1-((6-(1-methyl-1H-pyrazol-4-yl)pyrazolo[1,5-a]pyrazin-4-yl)oxy)bicyclo[4.1.0]heptan-3-ol

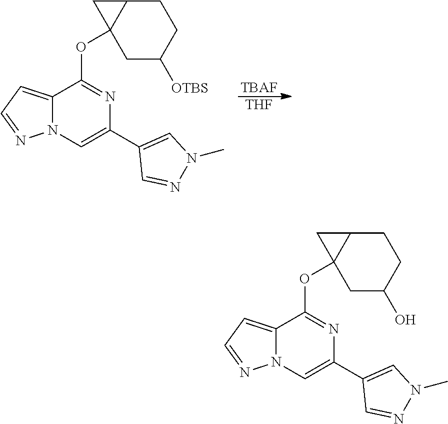 [SMILES: Cn1cc(-c2cn3nccc3c(OC34CC(O[Si](C)(C)C(C)(C)C)CCC3C4)n2)cn1 ; "TBAF" ; Cn1cc(-c2cn3nccc3c(OC34CC(O)CCC3C4)n2)cn1 ; "THF"]

A solution of 4-((3-((tert-butyldimethylsilyl)oxy)bicyclo[4.1.0]heptan-1-yl)oxy)-6-(1-methyl-1H-pyrazol-4-yl)pyrazolo[1,5-a]pyrazine (1.6 g, 3.64 mmol) in THF (100 mL) was added TBAF (1 M, 5.46 mL) and the reaction was stirred at 20° C. for 3 h. The mixture was concentrated in vacuo and the crude was purified by column chromatography on silica gel (PE/EtOAc=1/0 to 0/1) to give 1-((6-(1-methyl-1H-pyrazol-4-yl)pyrazolo[1,5-a]pyrazin-4-yl)oxy)bicyclo[4.1.0]heptan-3-ol (1.1 g, 3.93% yield) as colorless oil. LCMS m/z=326.2 (M+H)+

### 8. Synthesis of 1-((6-(1-methyl-1H-pyrazol-4-yl)pyrazolo[1,5-a]pyrazin-4-yl)oxy)bicyclo[4.1.0]heptan-3-one

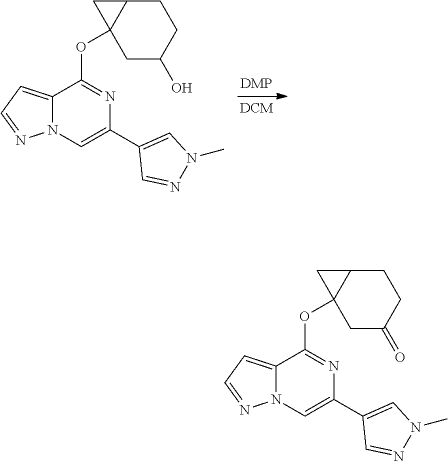 [SMILES: Cn1cc(-c2cn3nccc3c(OC34CC(O)CCC3C4)n2)cn1 ; Cn1cc(-c2cn3nccc3c(OC34CC(=O)CCC3C4)n2)cn1]

To a solution of 1-((6-(1-methyl-1H-pyrazol-4-yl)pyrazolo[1,5-a]pyrazin-4-yl)oxy)bicyclo[4.1.0]heptan-3-ol (1 g, 3.07 mmol) in DCM (50 mL) was added Dess-Martin periodinane (1.79 g, 9.22 mmol) and the reaction was stirred at 20° C. for 3 h. The mixture was filtered and concentrated under vacuum. The crude was purified by column chromatography on silica gel (PE/EtOAc=I/O to 0/1) to give 1-((6-(1-methyl-1H-pyrazol-4-yl)pyrazolo[1,5-a]pyrazin-4-yl)oxy)bicyclo[4.1.0]heptan-3-one (900 mg, 68% yield) as yellow solid. LCMS m/z=324.2 (M+H)+

### 9. Synthesis of N-methyl-1-((6-(1-methyl-1H-pyrazol-4-yl)pyrazolo[1,5-a]pyrazin-4-yl)oxy)bicyclo[4.1.0]heptan-3-amine

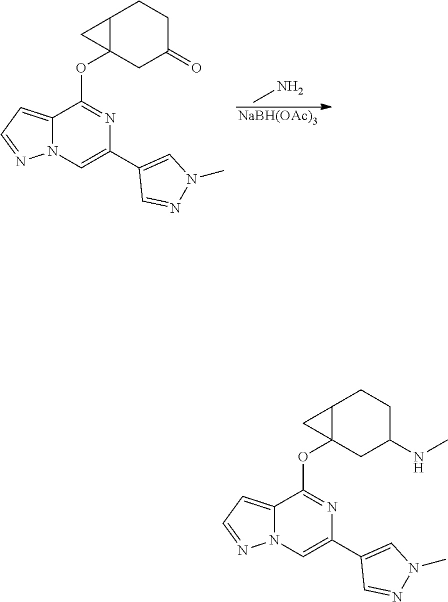

To a mixture of 1-((6-(1-methyl-1H-pyrazol-4-yl)pyrazolo[1,5-a]pyrazin-4-yl)oxy)bicyclo[4.1.0]heptan-3-one (1.00 g, 3.09 mmol) in MeOH (50 mL) was added DIPEA (1.20 g, 9.3 mmol) and methylamine hydrochloride (1.04 g, 15.5 mmol) at 0° C. and the reaction stirred for 15 min. NaBH(OAc)₃ (1.97 g, 9.28 mmol) was added and the reaction was stirred at 0° C. for 8 h. The reaction mixture was concentrated under vacuum to give N-methyl-1-((6-(1-methyl-1H-pyrazol-4-yl)pyrazolo[1,5-a]pyrazin-4-yl)oxy)bicyclo[4.1.0]heptan-3-amine (900 mg, crude), which was used in the next step directly. LCMS m/z=339.3 (M+H)+

### 10. Synthesis of tert-butyl methyl(1-((6-(1-methyl-1H-pyrazol-4-yl)pyrazolo[1,5-a]pyrazin-4-yl)oxy)bicyclo[4.1.0]heptan-3-yl)carbamate

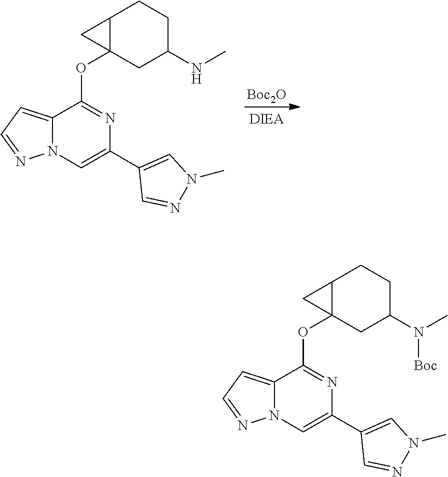 [SMILES: "Boc2O" ; CNC1CCC2CC2(Oc2nc(-c3cnn(C)c3)cn3nccc23)C1 ; CN(C(=O)OC(C)(C)C)C1CCC2CC2(Oc2nc(-c3cnn(C)c3)cn3nccc23)C1]

To a solution of N-methyl-1-((6-(1-methyl-1H-pyrazol-4-yl)pyrazolo[1,5-a]pyrazin-4-yl)oxy)bicyclo[4.1.0]heptan-3-amine (900 mg, 2.66 mmol) in MeOH (50 mL) was added DIPEA (1.35 g, 13.3 mmol) and Boc₂O (1.74 g, 7.98 mmol) and the reaction was stirred at 20° C. for 2 h. The reaction mixture was concentrated under vacuum and the crude product was purified by column chromatography (PE/EtOAc=0/1) to give tert-butyl methyl(1-((6-(1-methyl-1H-pyrazol-4-yl)pyrazolo[1,5-a]pyrazin-4-yl)oxy)bicyclo[4.1.0]heptan-3-yl)carbamate (700 mg, 60% yield) as yellow oil. LCMS m/z=439.3 (M+H)+

### 11. Synthesis of N-methyl-1-((6-(1-methyl-1H-pyrazol-4-yl)pyrazolo[1,5-a]pyrazin-4-yl)oxy)bicyclo[4.1.0]heptan-3-amine hydrochloride

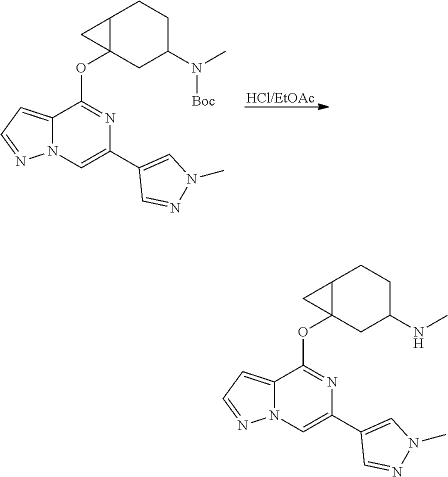 [SMILES: CN(C(=O)OC(C)(C)C)C1CCC2CC2(Oc2nc(-c3cnn(C)c3)cn3nccc23)C1 ; "HCl/EtOAc" ; CNC1CCC2CC2(Oc2nc(-c3cnn(C)c3)cn3nccc23)C1]

To a mixture of tert-butyl methyl(1-((6-(1-methyl-1H-pyrazol-4-yl)pyrazolo[1,5-a]pyrazin-4-yl)oxy)bicyclo[4.1.0]heptan-3-yl)carbamate (700 mg, 1.60 mmol) in DCM (50 mL) was added HCl/EtOAc (3 M, 8.75 mL) and the reaction mixture was stirred at 20° C. for 1 h. The reaction mixture was concentrated under vacuum to give N-methyl-1-((6-(1-methyl-1H-pyrazol-4-yl)pyrazolo[1,5-a]pyrazin-4-yl)oxy)bicyclo[4.1.0]heptan-3-amine hydrochloride (520 mg, crude), which was used in the next step directly. LCMS m/z=339.2 (M+H)+

### 12. Synthesis of N-methyl-N-(1-((6-(1-methyl-1H-pyrazol-4-yl)pyrazolo[1,5-a]pyrazin-4-yl)oxy)bicyclo[4.1.0]heptan-3-yl)but-2-ynamide

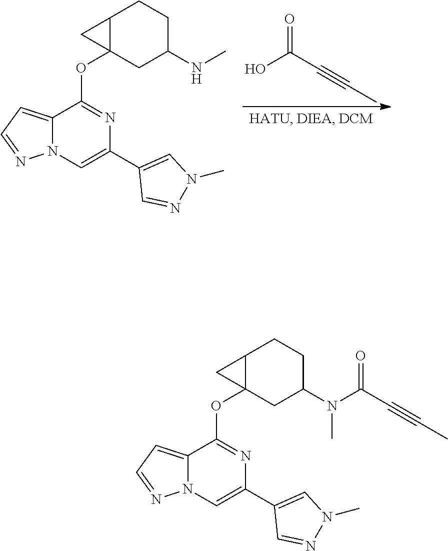

To a mixture of N-methyl-1-((6-(1-methyl-1H-pyrazol-4-yl)pyrazolo[1,5-a]pyrazin-4-yl)oxy)bicyclo[4.1.0]heptan-3-amine hydrochloride (650 mg, 1.92 mmol) in DCM (200 mL) was added DIPEA (744 mg, 5.76 mmol) and the reaction stirred at 20° C. for 15 min. But-2-ynoic acid (194 mg, 2.30 mmol) and HATU (879 mg, 2.30 mmol) were added and the reaction mixture was stirred at 20° C. for 2 h. The reaction mixture was concentrated under vacuum and the crude was purified by column chromatography on silica gel (PE/EtOAc=0/1) to give N-methyl-N-(1-((6-(1-methyl-1H-pyrazol-4-yl)pyrazolo[1,5-a]pyrazin-4-yl)oxy)bicyclo[4.1.0]heptan-3-yl)but-2-ynamide (600 mg, 77% yield) as colorless oil. LCMS m/z=405.3 (M+H)+

### 13. Synthesis of N-methyl-N—((1S,3S,6R)-1-((6-(1-methyl-1H-pyrazol-4-yl)pyrazolo[1,5-a]pyrazin-4-yl)oxy)bicyclo[4.1.0]heptan-3-yl)but-2-ynamide, N-methyl-N-((1S,3R,6R)-1-((6-(1-methyl-1H-pyrazol-4-yl)pyrazolo[1,5-a]pyrazin-4-yl)oxy)bicyclo[4.1.0]heptan-3-yl)but-2-ynamide, N-methyl-N-((1R,3R,6S)-1-((6-(1-methyl-1H-pyrazol-4-yl)pyrazolo[1,5-a]pyrazin-4-yl)oxy)bicyclo[4.1.0]heptan-3-yl)but-2-ynamide and N-methyl-N-((1R,3S,6S)-1-((6-(1-methyl-1H-pyrazol-4-yl)pyrazolo[1,5-a]pyrazin-4-yl)oxy)bicyclo[4.1.0]heptan-3-yl)but-2-ynamide

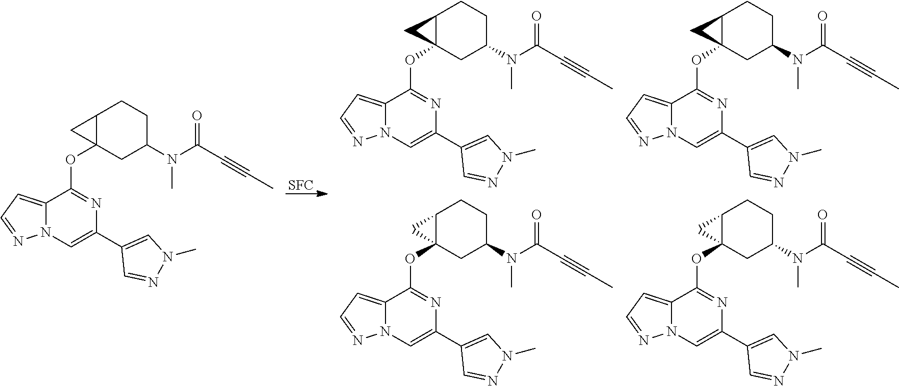

N-Methyl-N-(1-((6-(1-methyl-1H-pyrazol-4-yl)pyrazolo[1,5-a]pyrazin-4-yl)oxy)bicyclo[4.1.0]heptan-3-yl)but-2-ynamide (600 mg, 1.48 mmol) was separated by SFC (Column: DAICEL CHIRALPAK AD (250 mm×30 mm, 10 m); Condition: 35% [0.1% NH₃H₂O MeOH]; Flow Rate (mL/min) 70) to give:

### Example 113: The first eluting enantiomer, Peak 1, (82 mg, 14% yield) as a pale white solid, LCMS m/z=405.1 (M+H)+. 1H NMR (500 MHz, DMSO-d₆) δ=8.81-8.76 (m, 1H), 8.38-8.25 (m, 1H), 8.10-8.00 (m, 2H), 6.79 (d, J=17.0 Hz, 1H), 4.77-4.51 (m, 1H), 3.93 (d, J=6.0 Hz, 3H), 3.03-2.74 (m, 3H), 2.41-2.21 (m, 3H), 2.02-1.92 (m, 3H), 1.55 (s, 2H), 1.41 (s, 2H), 1.16-1.12 (m, 1H), 0.97-0.90 (m, 1H)

### Example 114: The second eluting enantiomer, Peak 2 (38 mg, 6% yield) as a pale white solid. LCMS m/z=405.2 (M+H)+. 1H NMR (500 MHz, DMSO-d₆) δ=8.77 (d, J=5.0 Hz, 1H), 8.16 (s, 1H), 8.02-7.98 (m, 2H), 6.78 (d, J=2.0 Hz, 1H), 4.14-4.05 (m, 1H), 3.91 (s, 3H), 3.02 (s, 1H), 2.78-2.73 (m, 3H), 2.37-2.33 (m, 2H), 2.14-2.01 (m, 3H), 1.85-1.75 (m, 1H), 1.57-1.48 (m, 3H), 1.28-1.24 (m, 1H), 0.82 (q, J=6.5 Hz, 1H)

### Example 116: The third eluting enatiomer, Peak 3 (100 mg, crude) as colorless oil. This was re-purified by SFC (Column: DAICEL CHIRALPAK AD (250 mm×30 mm, 10 μm); Condition: 45% [0.1% NH₃H₂O MeOH]; Flow Rate (mL/min) 80) (81 mg, as a pale white solid. LCMS m/z=405.1 (M+H)+. 1H NMR (500 MHz, DMSO-d₆) δ=8.81-8.76 (m, 1H), 8.37-8.24 (m, 1H), 8.10-8.00 (m, 2H), 6.79 (td, J=1.0, 17.0 Hz, 1H), 4.77-4.50 (m, 1H), 3.92 (d, J=6.0 Hz, 3H), 3.03-2.74 (m, 3H), 2.49-2.21 (m, 3H), 2.01 (s, 3H), 1.55 (s, 2H), 1.41 (s, 2H), 1.14 (d, J=6.0, 10.5 Hz, 1H), 0.97-0.90 (m, 1H)

### Example 115: And the fourth eluting enenatiomer, Peak 4 (50 mg, crude) as colorless oil. This was re-purified by SFC ((Column: DAICEL CHIRALPAK AD (250 mm×30 mm, 10 μm); Condition: 45% [0.1% NH₃H₂O MeOH]; Flow Rate (mL/min) 80) (32 mg, as a pale white solid). LCMS m/z=405.2 (M+H)+. 1H NMR (500 MHz, DMSO-d₆) δ=8.78 (d, J=5.0 Hz, 1H), 8.17 (s, 1H), 8.03-8.00 (m, 2H), 6.79 (d, J=2.0 Hz, 1H), 4.15-4.06 (m, 1H), 3.93 (s, 3H), 3.03 (s, 1H), 2.74 (s, 3H), 2.40-2.36 (m, 2H), 2.15-2.02 (m, 3H), 1.85-1.78 (m, 1H), 1.58-1.49 (m, 3H), 1.29-1.25 (m, 1H), 0.82 (q, J=6.5 Hz, 1H)

The absolute stereochemistry of the product in each peak was not assigned.

### Example 117. (S) or (R)-1-(2-methyl-2-(((6-(1-methyl-1H-pyrazol-4-yl)pyrazolo[1,5-a]pyrazin-4-yl)oxy)methyl)morpholino)prop-2-en-1-one

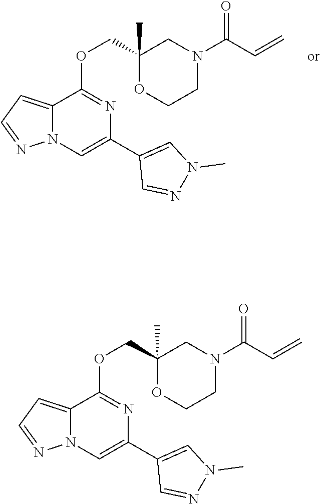 [SMILES: C=CC(=O)N1CCO[C@](C)(COc2nc(-c3cnn(C)c3)cn3nccc23)C1 ; C=CC(=O)N1CCO[C@@](C)(COc2nc(-c3cnn(C)c3)cn3nccc23)C1]

### 1. Synthesis of tert-butyl 2-(hydroxymethyl)-2-methylmorpholine-4-carboxylate

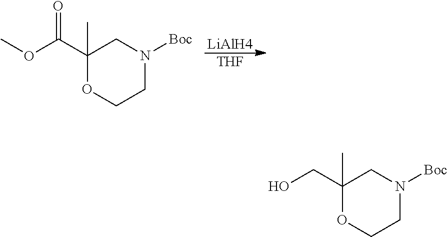

To a solution of 4-(tert-butyl) 2-methyl 2-methylmorpholine-2,4-dicarboxylate (800 mg, 3.09 mmol) in THF (5 mL) was added LiAlH₄ (234 mg, 6.17 mmol) at 0° C. and the reaction was stirred at 0° C. for 2 h. Water (0.5 mL) was added and the mixture dried over Na₂SO₄, filtered and evaporated to dryness to give tert-butyl 2-(hydroxymethyl)-2-methylmorpholine-4-carboxylate (500 mg, crude) as a yellow solid. LCMS m/z=176.3 (M+H)+

### 2. Synthesis of tert-butyl 2-(((6-chloropyrazolo[1,5-a]pyrazin-4-yl)oxy)methyl)-2-methylmorpholine-4-carboxylate

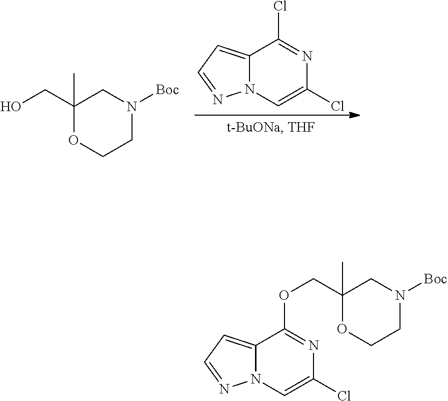

To a solution of tert-butyl 2-(hydroxymethyl)-2-methylmorpholine-4-carboxylate (500 mg, 2.16 mmol) in THF (15 mL) was added t-BuONa (416 mg, 4.32 mmol) at 0° C. and the mixture was stirred for 10 mins. 4,6-Dichloropyrazolo[1,5-a]pyrazine (406 mg, 2.16 mmol) was added and the reaction stirred at 0° C. for 30 mins. The mixture was concentrated under vacuum and the residue was purified by silica gel chromatography (from PE to PE/EtOAc=2/1) to give tert-butyl 2-(((6-chloropyrazolo[1,5-a]pyrazin-4-yl)oxy)methyl)-2-methylmorpholine-4-carboxylate (340 mg, 41% yield) as a white solid. LCMS m/z=383.1 (M+H)+

### 3. Synthesis of tert-butyl 2-methyl-2-(((6-(1-methyl-1H-pyrazol-4-yl)pyrazolo[1,5-a]pyrazin-4-yl)oxy)methyl)morpholine-4-carboxylate

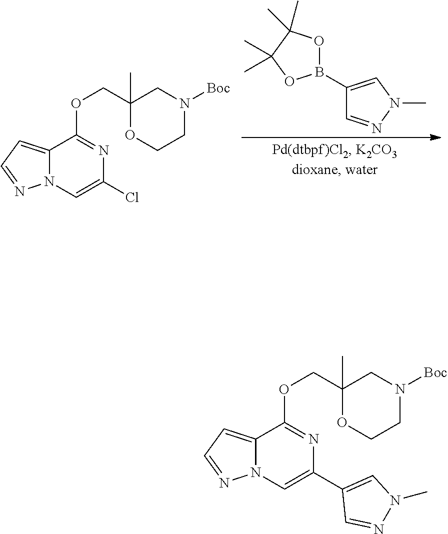

To a solution of tert-butyl 2-(((6-chloropyrazolo[1,5-a]pyrazin-4-yl)oxy)methyl)-2-methylmorpholine-4-carboxylate (490 mg, 1.28 mmol) in dioxane (12 mL) and water (2 mL) was added 1-methyl-4-(4,4,5,5-tetramethyl-1,3,2-dioxaborolan-2-yl)-1H-pyrazole (533 mg, 2.56 mmol), K₂CO₃ (531 mg, 3.84 mmol) and Pd(dtbpf)Cl₂ (83 mg, 128 μmol) and the reaction stirred at 90° C. under N₂ for 2 h. The mixture was concentrated under vacuum and the residue was purified by silica gel chromatography (from PE to EtOAc) to give tert-butyl 2-methyl-2-(((6-(1-methyl-1H-pyrazol-4-yl)pyrazolo[1,5-a]pyrazin-4-yl)oxy)methyl)morpholine-4-carboxylate (460 mg, 84% yield) as yellow oil.

### 4. Synthesis of tert-butyl (R) and (S)-2-methyl-2-(((6-(1-methyl-1H-pyrazol-4-yl)pyrazolo[1,5-a]pyrazin-4-yl)oxy)methyl)morpholine-4-carboxylate

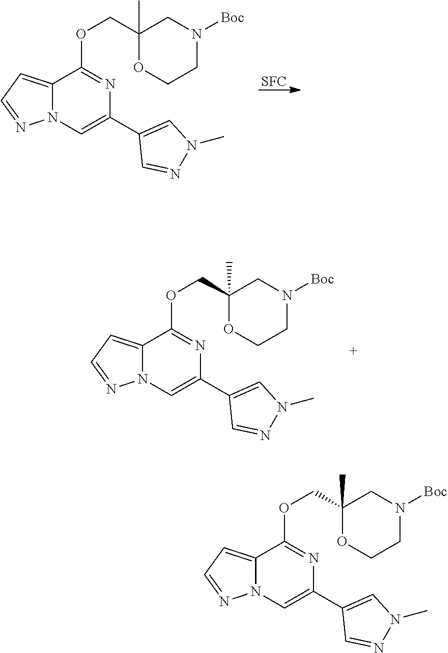

tert-Butyl 2-methyl-2-(((6-(1-methyl-1H-pyrazol-4-yl)pyrazolo[1,5-a]pyrazin-4-yl)oxy)methyl)morpholine-4-carboxylate (330 mg, 770 μmol) was purified by SFC (Column: DAICEL CHIRALPAK AD (250 mm×30 mm, 10 μm); Condition: 25%[0.1% NH₃H₂O EtOH], Flow Rate (mL/min): 60) to give:
the first eluting peak (E1), peak 1 (120 mg, 36% yield) as a white solid. LCMS m/z=429.2 (M+H)+
and the second eluting peak (E2), peak 2 (120 mg, 36% yield) as a white solid. LCMS m/z=429.2 (M+H)+. The absolute stereochemistry of the product in each peak was not assigned.

### 5. Synthesis of (S) or (R)-2-methyl-2-(((6-(1-methyl-1H-pyrazol-4-yl)pyrazolo[1,5-a]pyrazin-4-yl)oxy)methyl)morpholine hydrochloride

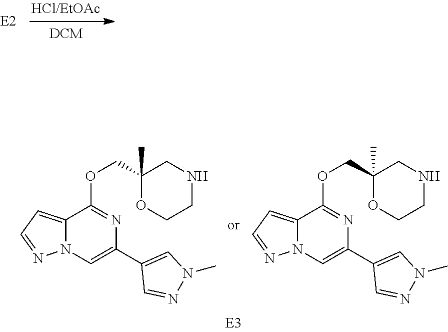 [SMILES: Cn1cc(-c2cn3nccc3c(OC[C@]3(C)CNCCO3)n2)cn1 ; Cn1cc(-c2cn3nccc3c(OC[C@@]3(C)CNCCO3)n2)cn1]

To a solution of E2, tert-butyl (S) or (R)-2-methyl-2-(((6-(1-methyl-1H-pyrazol-4-yl)pyrazolo[1,5-a]pyrazin-4-yl)oxy)methyl)morpholine-4-carboxylate (120 mg, 280 mmol) in DCM (10 mL) was added HCl/EtOAc (8 mL, 4 M) and the reaction stirred at 15° C. for 30 mins. The mixture was concentrated under vacuum to give (S) or (R)-2-methyl-2-(((6-(1-methyl-1H-pyrazol-4-yl)pyrazolo[1,5-a]pyrazin-4-yl)oxy)methyl)morpholine hydrochloride (100 mg, crude) as a white solid. LCMS m/z=329.1 (M+H)+

### 6. Synthesis of (S) or (R)-1-(2-methyl-2-(((6-(1-methyl-1H-pyrazol-4-yl)pyrazolo[1,5-a]pyrazin-4-yl)oxy)methyl)morpholino)prop-2-en-1-one

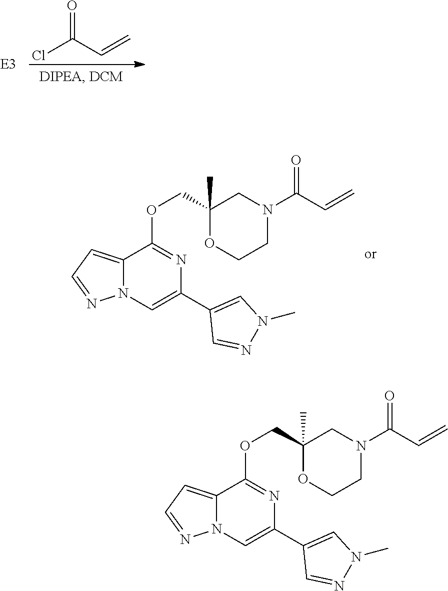

To a solution of E3, (S) or (R)-2-methyl-2-(((6-(1-methyl-1H-pyrazol-4-yl)pyrazolo[1,5-a]pyrazin-4-yl)oxy)methyl)morpholine hydrochloride (100 mg, 274 μmol) in DCM (30 mL) was added DIPEA (71 mg, 548 μmol) and acryloyl chloride (25 mg, 274 μmol) at 0° C. and the reaction stirred at 0° C. for 10 min. The mixture was quenched with MeOH (2 mL) and concentrated under vacuum. The residue was purified by prep-HPLC (Column: Welch Xtimate C18 150×25 mm×5 μm; Condition: water (10 mM NH₄HCO₃)-MeCN, Begin B 20 End B 50, Gradient Time (min) 10, Flow Rate (mL/min): 25) to give (S) or (R)-1-(2-methyl-2-(((6-(1-methyl-1H-pyrazol-4-yl)pyrazolo[1,5-a]pyrazin-4-yl)oxy)methyl)morpholino)prop-2-en-1-one (76 mg, 73% yield) as a yellow solid. LCMS m/z=383.1 (M+H)+. 1H NMR (500 MHz, DMSO-d₆) δ=8.77 (s, 1H), 8.19 (d, J=8.0 Hz, 1H), 8.03 (d, J=2.0 Hz, 1H), 8.00 (s, 1H), 6.96-6.77 (m, 2H), 6.14 (t, J=18.5 Hz, 1H), 5.74-5.59 (m, 1H), 4.65-4.48 (m, 2H), 3.88 (s, 3H), 3.78-3.58 (m, 6H), 1.29 (s, 3H).

### Example 118. (R) or (S)-1-(2-methyl-2-(((6-(1-methyl-1H-pyrazol-4-yl)pyrazolo[1,5-a]pyrazin-4-yl)oxy)methyl)morpholino)prop-2-en-1-one

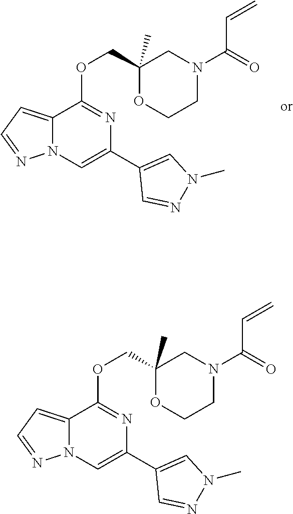 [SMILES: C=CC(=O)N1CCO[C@@](C)(COc2nc(-c3cnn(C)c3)cn3nccc23)C1 ; C=CC(=O)N1CCO[C@](C)(COc2nc(-c3cnn(C)c3)cn3nccc23)C1]

(R) or (S)-1-(2-methyl-2-(((6-(1-methyl-1H-pyrazol-4-yl)pyrazolo[1,5-a]pyrazin-4-yl)oxy)methyl)morpholino)prop-2-en-1-one was obtained as a white solid, from E1, tert-butyl (R) or (S)-2-methyl-2-(((6-(1-methyl-1H-pyrazol-4-yl)pyrazolo[1,5-a]pyrazin-4-yl)oxy)methyl)morpholine-4-carboxylate and acryloyl chloride, following the procedure described in Example 117. LCMS m/z=383.2 (M+H)+. 1H NMR (500 MHz, DMSO-d₆) δ=8.77 (s, 1H), 8.19 (d, J=7.5 Hz, 1H), 8.04-8.00 (m, 2H), 6.96-6.88 (m, 2H), 6.14 (s, 1H), 5.74-5.59 (m, 1H), 4.56-4.49 (m, 2H), 3.88 (s, 3H), 3.78-3.58 (m, 6H), 1.29 (s, 3H).

### Examples 119 & 120. (S) and (R)-1-(2,2,6-trimethyl-6-(((6-(1-methyl-1H-pyrazol-4-yl)pyrazolo[1,5-a]pyrazin-4-yl)oxy)methyl)morpholino)prop-2-en-1-one

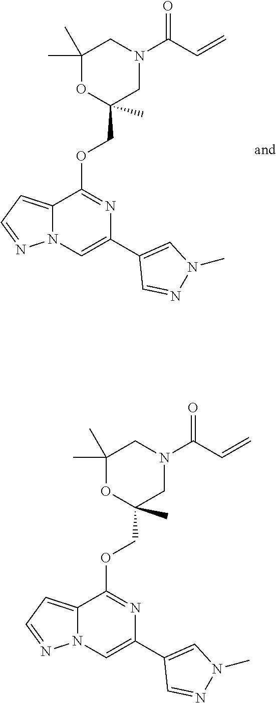 [SMILES: C=CC(=O)N1CC(C)(C)O[C@](C)(COc2nc(-c3cnn(C)c3)cn3nccc23)C1 ; C=CC(=O)N1CC(C)(C)O[C@@](C)(COc2nc(-c3cnn(C)c3)cn3nccc23)C1]

### 1. Synthesis of 4-(tert-butoxycarbonyl)-6,6-dimethylmorpholine-2-carboxylic acid

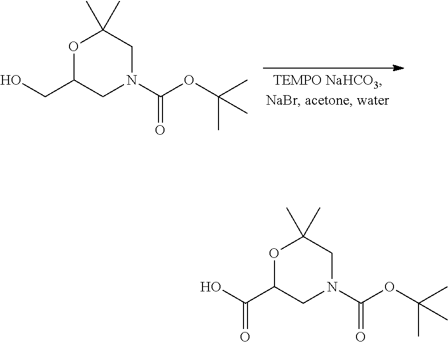 [SMILES: CC(C)(C)OC(=O)N1CC(CO)OC(C)(C)C1 ; CC(C)(C)OC(=O)N1CC(C(=O)O)OC(C)(C)C1]

To a solution of tert-butyl 6-(hydroxymethyl)-2,2-dimethylmorpholine-4-carboxylate (1 g, 4.08 mmol) in acetone (40 mL) was added a solution of sat. NaHCO₃ (12 mL) at 0° C., followed by NaBr (84 mg, 815 μmol) and TEMPO (13 mg, 81 μmol) and the solution then cooled to 0° C. 1,3,5-Trichloro-1,3,5-triazinane-2,4,6-trione (1.89 g, 8.15 mmol) was added in portions and the reaction stirred at RT for 12 h. IPA (3 mL) was added and the resulting solution was stirred at 25° C. for 2 h. The mixture was filtered and the filtrate was basified to pH 8 using sat Na₂CO₃ solution. The resulting mixture was concentrated in vacuo and lyophilized to give 4-(tert-butoxycarbonyl)-6,6-dimethylmorpholine-2-carboxylic acid (1 g, crude) as a white solid. LCMS m/z=204.1 (M+H)+

### 2. Synthesis of 4-(tert-butyl) 2-methyl 6,6-dimethylmorpholine-2,4-dicarboxylate

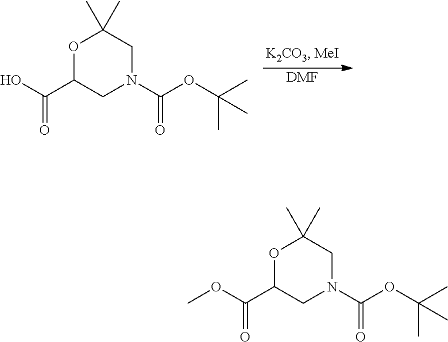

To a mixture of 4-(tert-butoxycarbonyl)-6,6-dimethylmorpholine-2-carboxylic acid (1 g, 3.86 mmol) and K₂CO₃ (1.60 g, 11.57 mmol) in DMF (15 mL) was added Mel (821 mg, 5.78 mmol) and the reaction was stirred at 15° C. for 16 h. The mixture was concentrated in vacuo and the crude product, was purified by CombiFlash® eluting with EtOAc in PE from 0% to 30% to give 4-(tert-butyl) 2-methyl 6,6-dimethylmorpholine-2,4-dicarboxylate (750 mg, 64% yield) as a white solid. 1H NMR (500 MHz, CDCl₃) δ=4.41-4.09 (m, 2H), 3.77 (s, 4H), 2.94-2.60 (m, 2H), 1.47 (s, 9H), 1.30-1.23 (m, 6H).

### 3. Synthesis of 4-(tert-butyl) 2-methyl 2,6,6-trimethylmorpholine-2,4-dicarboxylate

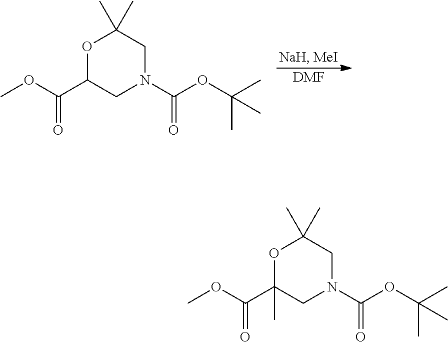

To a solution of 4-(tert-butyl) 2-methyl 6,6-dimethylmorpholine-2,4-dicarboxylate (200 mg, 731 μmol) in DMF (3 mL) was added NaH (88 mg, 2.20 mmol) at 0° C. under N₂ and the mixture was stirred at 18° C. for 1 h. The mixture was re-cooled to 0° C., then Mel (208 mg, 1.46 mmol) was added. The reaction was allowed to warm to 18° C. and stirred for 8 h. Sat. NH₄Cl aq. (2 mL) was added dropwise and the mixture was neutralized with 1M HCl. The mixture was extracted with EtOAc (15 mL×3), the organics were washed with water (10 mL×4), dried over Na₂SO₄, filtered and concentrated in vacuo. The crude was purified by Combiflash® eluting with EtOAc in PE from 0% to 30%. The product was further purified by Combiflash® eluting with EtOAc in PE from 0% to 30% to give 4-(tert-butyl) 2-methyl 2,6,6-trimethylmorpholine-2,4-dicarboxylate as a colorless oil. LCMS m/z=232.1 (M+H)+

### 4. Synthesis of tert-butyl 2-(hydroxymethyl)-2,6,6-trimethylmorpholine-4-carboxylate

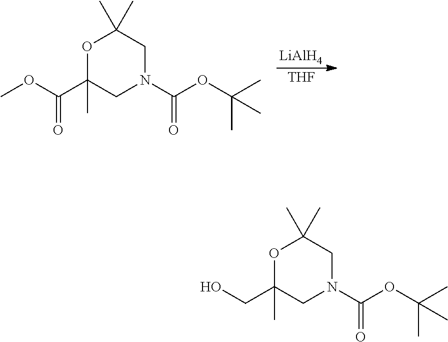 [SMILES: COC(=O)C1(C)CN(C(=O)OC(C)(C)C)CC(C)(C)O1 ; "LiAlH4" ; CC(C)(C)OC(=O)N1CC(C)(C)OC(C)(CO)C1 ; "THF"]

To a mixture of 4-(tert-butyl) 2-methyl 2,6,6-trimethylmorpholine-2,4-dicarboxylate (96 mg, 334 μmol) in THF (20 mL) was added LiAlH₄ (32 mg, 843 μmol) at 0° C. under N₂ and the mixture was stirred at 0° C. for 1 h. Water (1 mL) was added dropwise and the mixture was filtered. The filtrate was evaporated under reduced pressure to give tert-butyl 2-(hydroxymethyl)-2,6,6-trimethylmorpholine-4-carboxylate (90 mg, crude) as a colorless oil. LCMS m/z=204.1 (M+H)+

### 5. Synthesis of tert-butyl 2-(hydroxymethyl)-2,6,6-trimethylmorpholine-4-carboxylate

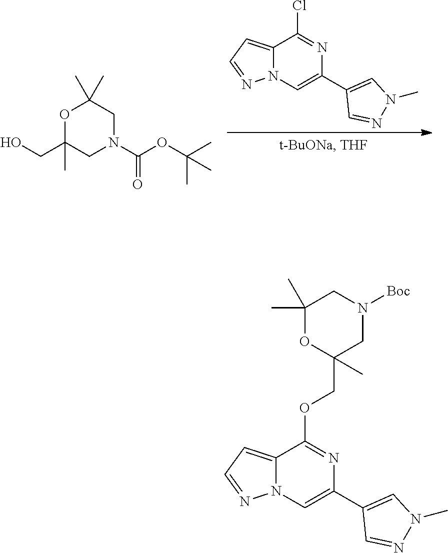 [SMILES: CC(C)(C)OC(=O)N1CC(C)(C)OC(C)(CO)C1 ; Cn1cc(-c2cn3nccc3c(Cl)n2)cn1 ; Cn1cc(-c2cn3nccc3c(OCC3(C)CN(C(=O)OC(C)(C)C)CC(C)(C)O3)n2)cn1]

tBuONa (111 mg, 1.16 mmol) was added to a mixture of tert-butyl 2-(hydroxymethyl)-2,6,6-trimethylmorpholine-4-carboxylate (100 mg, 385 μmol) and 4-chloro-6-(1-methyl-1H-pyrazol-4-yl)pyrazolo[1,5-a]pyrazine (Intermediate A, 90 mg, 385 μmol) in THF (5 mL) and the reaction stirred at 25° C. for 12 h and at 45° C. for 5 h. The reaction was concentrated in vacuo and the crude product was purified by Combiflash® eluting with 0% to 50% to give tert-butyl 2-(hydroxymethyl)-2,6,6-trimethylmorpholine-4-carboxylate (140 mg, 44% yield) as a white gum. LCMS m/z=457.3 (M+H)+

### 6. Synthesis of tert-butyl 2-(hydroxymethyl)-2,6,6-trimethylmorpholine-4-carboxylate hydrochloride

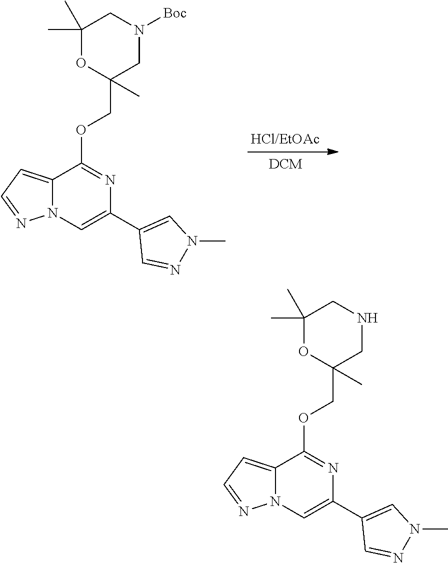 [SMILES: Cn1cc(-c2cn3nccc3c(OCC3(C)CN(C(=O)OC(C)(C)C)CC(C)(C)O3)n2)cn1 ; Cn1cc(-c2cn3nccc3c(OCC3(C)CNCC(C)(C)O3)n2)cn1]

HCl in EtOAc (4 M, 8 mL) was added to a solution of tert-butyl 2-(hydroxymethyl)-2,6,6-trimethylmorpholine-4-carboxylate (120 mg, 262 μmol) in DCM (2 mL) and the reaction was stirred at 25° C. for 1 h. The reaction mixture was evaporated under reduced pressure to give tert-butyl 2-(hydroxymethyl)-2,6,6-trimethylmorpholine-4-carboxylate hydrochloride (93 mg, crude) as a yellow solid, which was used in the next step directly. LCMS m/z=357.2 (M+H)+

### 7. Synthesis of 1-(2,2,6-trimethyl-6-(((6-(1-methyl-1H-pyrazol-4-yl)pyrazolo[1,5-a]pyrazin-4-yl)oxy)methyl)morpholino)prop-2-en-1-one

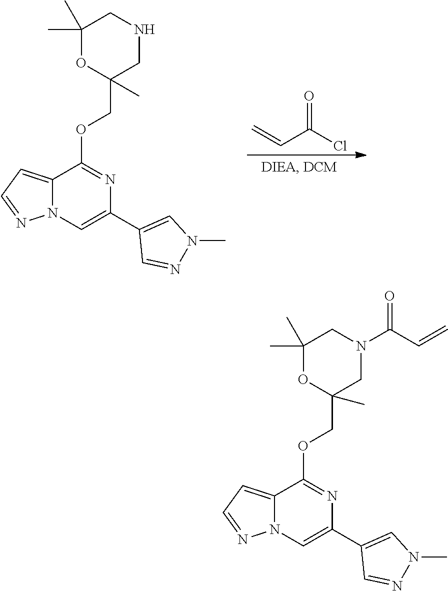

To a mixture of tert-butyl 2-(hydroxymethyl)-2,6,6-trimethylmorpholine-4-carboxylate hydrochloride (93 mg, 169 μmol) and DIPEA (66 mg, 508 μmol) in DCM (6 mL) was added acryloyl chloride (18 mg, 203 μmol) at 0° C. and the reaction stirred for 2 min. MeOH (1 mL) was added dropwise and the mixture was stirred at 25° C. for 10 min. The solvent was removed in vacuo and the crude was purified by prep HPLC (Column: Boston Prime C18 150×30 mm×5 um; Condition: water (0.05% NH₃H₂O+10 mM NH₄HCO₃)-MeCN, Begin B 32, End B 47, Gradient Time (min) 14, Flow Rate (mL/min) 25) to give 1-(2,2,6-trimethyl-6-(((6-(1-methyl-1H-pyrazol-4-yl)pyrazolo[1,5-a]pyrazin-4-yl)oxy)methyl)morpholino)prop-2-en-1-one (50 mg, 68% yield) as a white solid. LCMS m/z=411.2 (M+H)+

### 8. Synthesis of (S) and (R)-1-(2,2,6-trimethyl-6-(((6-(1-methyl-1H-pyrazol-4-yl)pyrazolo[1,5-a]pyrazin-4-yl)oxy)methyl)morpholino)prop-2-en-1-one

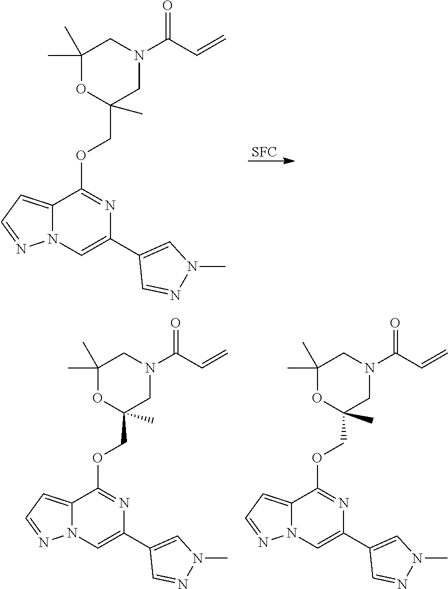

1-(2,2,6-Trimethyl-6-(((6-(1-methyl-1H-pyrazol-4-yl)pyrazolo[1,5-a]pyrazin-4-yl)oxy)methyl)morpholino)prop-2-en-1-one (50 mg, 121 μmol) was separated by SFC (Column: DAICEL CHIRALPAK AD (250 mm×30 mm, 10 um); Condition:40%[0.1% NH₃H₂O EtOH], Gradient Time (min), Flow Rate (mL/min) 80) to give

### Example 119: The first eluting enantiomer, Peak 1 (23.4 mg, 46.8% yield) as a white solid. LCMS m/z=411.1 (M+H)⁺. ¹H NMR: (400 MHz, MeOH-d₄) δ=8.45 (d, J=3.2 Hz, 1H), 8.08 (d, J=8.0 Hz, 1H), 7.94 (s, 2H), 6.93-6.70 (m, 2H), 6.33-6.13 (m, 1H), 5.84-5.60 (m, 1H), 4.67-4.32 (m, 2H), 3.94 (s, 3H), 3.87-3.52 (m, 4H), 1.39 (s, 3H), 1.30-1.18 (m, 6H)

### Example 120: And the second eluting enantiomer, Peak 2 (19.4 mg, 38.8% yield) as a pale yellow solid. LCMS m/z=411.1 (M+H)⁺. ¹H NMR: (400 MHz, MeOH-d₄) δ=8.45 (d, J=3.2 Hz, 1H), 8.08 (d, J=8.0 Hz, 1H), 7.94 (s, 2H), 6.93-6.70 (m, 2H), 6.33-6.13 (m, 1H), 5.84-5.60 (m, 1H), 4.67-4.32 (m, 2H), 3.94 (s, 3H), 3.87-3.52 (m, 4H), 1.39 (s, 3H), 1.30-1.18 (m, 6H)

The absolute stereochemistry of each peak was not assigned.

### Examples 121 & 122. (S) and (R)-1-(2,2,6-trimethyl-6-(((6-(1-methyl-1H-pyrazol-4-yl)pyrazolo[1,5-a]pyrazin-4-yl)oxy)methyl)morpholino)but-2-yn-1-one

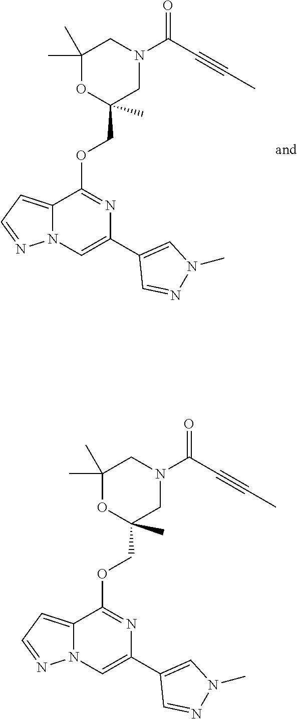 [SMILES: CC#CC(=O)N1CC(C)(C)O[C@](C)(COc2nc(-c3cnn(C)c3)cn3nccc23)C1 ; CC#CC(=O)N1CC(C)(C)O[C@@](C)(COc2nc(-c3cnn(C)c3)cn3nccc23)C1]

1-(2,2,6-Trimethyl-6-(((6-(1-methyl-1H-pyrazol-4-yl)pyrazolo[1,5-a]pyrazin-4-yl)oxy)methyl)morpholino)but-2-yn-1-one was obtained from tert-butyl 2-(hydroxymethyl)-2,6,6-trimethylmorpholine-4-carboxylate hydrochloride and but-2-ynoic acid, following the procedure described in step 7, Example 119. The compound was further purified by SFC (Column: DAICEL CHIRALCEL OJ (250 mm×30 mm, 10 um); Condition: 20% [0.1% NH₃H₂O EtOH], Flow Rate (mL/min) 60) to give:

### Example 121: The first eluting enantiomer 1, Peak 1 (32 mg, 29% yield) as a white solid. LCMS m/z=423.3 (M+H)+. 1H NMR (400 MHz, MeOH-d₄) δ=8.46 (d, J=12.8 Hz, 1H), 8.08 (d, J=9.2 Hz, 1H), 7.97-7.92 (m, 2H), 6.90-6.85 (m, 1H), 4.86-4.40 (m, 1H), 4.38-4.10 (m, 1H), 3.94 (s, 3H), 3.78-3.57 (m, 3H), 3.37-3.34 (m, 1H), 2.07-1.63 (m, 3H), 1.46-1.24 (m, 9H)

### Example 122: and the second eluting enantiomer, Peak 2 (40 mg, 38% yield) as a pale yellow solid. LCMS m/z=423.3 (M+H)+. 1H NMR: (400 MHz, MeOH-d₄) δ=8.46 (d, J=12.8 Hz, 1H), 8.08 (d, J=9.2 Hz, 1H), 7.97-7.92 (m, 2H), 6.90-6.85 (m, 1H), 4.86-4.40 (m, 1H), 4.38-4.10 (m, 1H), 3.94 (s, 3H), 3.78-3.57 (m, 3H), 3.37-3.34 (m, 1H), 2.07-1.63 (m, 3H), 1.46-1.24 (m, 9H)

The absolute stereochemistry of each product in each peak was not assigned.

### Example 123. N-methyl-N-((1S,2S,4R,6R)-6-((6-(1-methyl-1H-pyrazol-4-yl)pyrazolo[1,5-a]pyrazin-4-yl)oxy)bicyclo[2.2.1]heptan-2-yl)but-2-ynamide and N-methyl-N-((1R,2R,4S,6S)-6-((6-(1-methyl-1H-pyrazol-4-y)pyrazolo[1,5-a]pyrazin-4-yl)oxy)bicyclo[2.2.1]heptan-2-y)but-2-ynamide

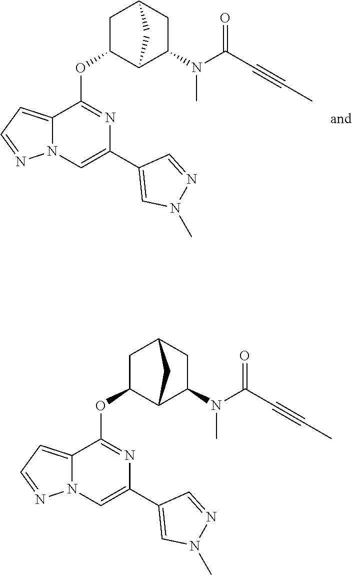 [SMILES: CC#CC(=O)N(C)[C@H]1C[C@H]2C[C@@H]1[C@H](Oc1nc(-c3cnn(C)c3)cn3nccc13)C2 ; CC#CC(=O)N(C)[C@@H]1C[C@@H]2C[C@H]1[C@@H](Oc1nc(-c3cnn(C)c3)cn3nccc13)C2]

### 1. Synthesis of 2-(bicyclo[2.2.1]hept-5-en-2-yl)isoindoline-1,3-dione

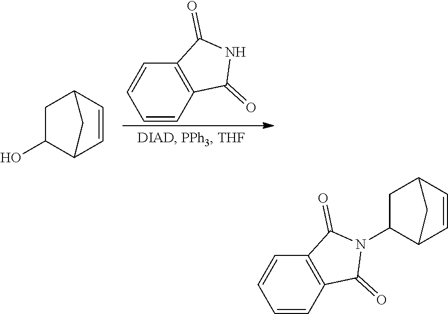

To a solution of bicyclo[2.2.1]hept-5-en-2-ol (15 g, 136 mmol) in THF (400 mL) was added 2-(bicyclo[2.2.1]hept-5-en-2-yl)isoindoline-1,3-dione (26.05 g, 177 mmol) and PPh₃ (53.58 g, 204 mmol) at 15° C. DIAD (41.3 g, 204 mmol) was added and the reaction was stirred at 15° C. for 8 h. The mixture was filtered and concentrated under vacuum and the crude was purified by column chromatography on silica gel (PE/EtOAc=1/0 to 5/1) to give 2-(bicyclo[2.2.1]hept-5-en-2-yl)isoindoline-1,3-dione (13 g, 40% yield) as a white solid. LCMS m/z=240.2 (M+H)+

### 2. Synthesis of racemic-2-((1S,2R,4S,5R)-5-hydroxybicyclo[2.2.1]heptan-2-yl)isoindoline-1,3-dione, racemic-2-((1S,2R,4S,5S)-5-hydroxybicyclo[2.2.1]heptan-2-yl)isoindoline-1,3-dione, racemic-2-((1R,2R,4S,6S)-6-hydroxybicyclo[2.2.1]heptan-2-yl)isoindoline-1,3-dione and racemic-2-((1R,2R,4S,6R)-6-hydroxybicyclo[2.2.1]heptan-2-yl)isoindoline-1,3-dione

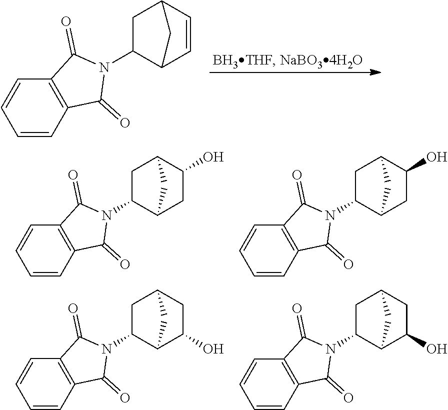

To a solution of 2-(bicyclo[2.2.1]hept-5-en-2-yl)isoindoline-1,3-dione (2 g, 8.36 mmol) in THF (60 mL) was added BH₃.THF (2.87 g, 33.4 mmol) dropwise at 0° C. and the reaction stirred under N₂ at 0° C. for 2 h. Water (20 mL) was carefully added followed by a suspension of NaBO₃·4H₂O (3.86 g, 25 mmol) and the mixture was vigorously stirred at 20° C. for 12 h. The mixture was concentrated under vacuum and the crude product was purified by column chromatography (PE/EtOAc=7/3). The product was purified by prep HPLC (Column: Phenomenex Genimi NX C18 150×40 mm×5 um; Condition: water (10 mM NH₄HCO₃)-MeCN; Begin B 23, End B 43, Gradient Time (min) 10, Flow Rate (mL/min) 60) to give:

The first eluting enantiomer, Peak 1, (E1), (500 mg, 23% yield) as a white solid, 1H NMR (400 MHz, DMSO-d₆) δ=7.77 (s, 4H), 4.58 (s, 1H), 3.84-3.81 (m, 1H), 3.61 (d, J=5.6 Hz, 1H), 2.33 (d, J=4.4 Hz, 1H), 2.15-2.11 (m, 2H), 1.96 (s, 1H), 1.51-1.44 (m, 2H), 1.37-1.31 (m, 1H), 1.21-1.18 (m, 1H).

The second eluting enantiomer, Peak 2, (E2) (100 mg, 5% yield) as a brown solid. LCMS m/z=258.1 (M+H)+

The third eluting enantiomer, Peak 3, (E3), (900 mg, 42% yield) as a white solid. 1H NMR (400 MHz, DMSO-d₆) δ=7.80-7.75 (m, 4H), 4.67 (s, 1H), 3.84-3.80 (m, 1H), 3.66 (d, J=6.4 Hz, 1H), 2.28 (s, 1H), 2.19 (s, 1H), 2.04-1.97 (m, 2H), 1.55-1.51 (m, 2H), 1.45-1.39 (m, 1H), 1.19 (d, J=13.2 Hz, 1H).

and the fourth eluting enantiomer, Peak 4, (E4), (150 mg, 7% yield) as a brown solid. LCMS m/z=240.1 (M+H-18)+

### 3. Synthesis of racemic-(1R,2S,4S,6R)-6-aminobicyclo[2.2.1]heptan-2-ol

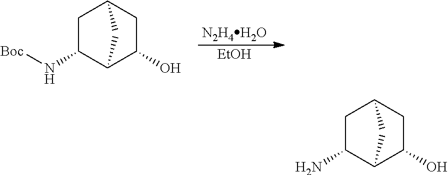

To a solution of E3, (0.55 g, 2.14 mmol) in EtOH (30 mL) was added N₂H₄·H₂O (2 mL, 85% purity) and the mixture was stirred at 20° C. for 30 min. MTBE (30 mL) was added and the mixture stirred at 20° C. for 10 min. The mixture was filtered and the filter cake was dissolved in DCM. The mixture was filtered and the combined organic layers were concentrated under vacuum to give racemic-(1R,2S,4S,6R)-6-aminobicyclo[2.2.1]heptan-2-ol (0.27 g, crude) as a white solid. LCMS m/z=128.1 (M+H)+

### 4. Synthesis of racemic-tert-butyl ((1R,2R,4S,6S)-6-hydroxybicyclo[2.2.1]heptan-2-yl)carbamate

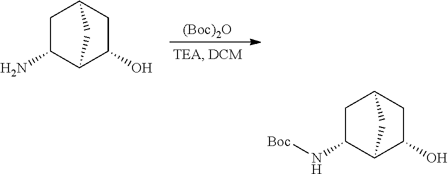 [SMILES: "(Boc)2O" ; N[C@@H]1C[C@@H]2C[C@H]1[C@@H](O)C2 ; CC(C)(C)OC(=O)N[C@@H]1C[C@@H]2C[C@H]1[C@@H](O)C2]

To a solution of racemic-(1R,2S,4S,6R)-6-aminobicyclo[2.2.1]heptan-2-ol (0.27 g, 2.12 mmol) in DCM (10 mL) was added TEA (644 mg, 6.37 mmol) and (Boc)₂O (1.39 g, 6.37 mmol) and the reaction was stirred at 20° C. for 3 h. The reaction mixture was concentrated under vacuum and the crude product was purified by column chromatography (PE/EtOAc=1/1) to give racemic-tert-butyl ((1R,2R,4S,6S)-6-hydroxybicyclo[2.2.1]heptan-2-yl)carbamate (325 mg, 61% yield) as a white solid. LCMS m/z=228.3 (M+H)⁺. 1HNMR (400 MHz, DMSO-d₆) δ=6.74 (d, J=7.2 Hz, 1H), 4.48 (d, J=3.6 Hz, 1H), 3.53 (s, 1H), 3.12 (s, 1H), 2.09 (s, 1H), 1.89 (s, 1H), 1.44-1.40 (m, 2H), 1.37 (s, 9H), 1.34 (s, 1H), 1.25 (d, J=9.2 Hz, 1H), 1.17-1.10 (m, 2H).

### 5. Synthesis of racemic-tert-butyl ((1R,2R,4S,6S)-6-((6-(1-methyl-1H-pyrazol-4-yl)pyrazolo[1,5-a]pyrazin-4-yl)oxy)bicyclo[2.2.1]heptan-2-yl)carbamate

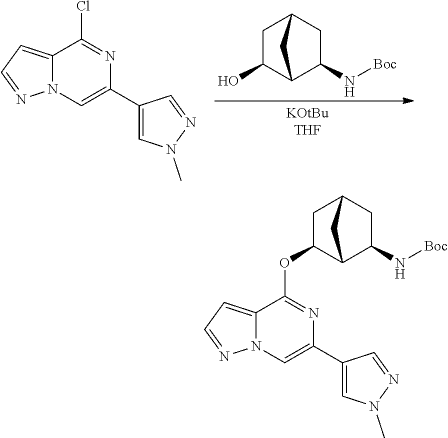 [SMILES: Cn1cc(-c2cn3nccc3c(Cl)n2)cn1 ; CC(C)(C)OC(=O)N[C@@H]1C[C@@H]2C[C@H]1[C@@H](O)C2 ; Cn1cc(-c2cn3nccc3c(O[C@H]3C[C@H]4C[C@@H]3[C@H](NC(=O)OC(C)(C)C)C4)n2)cn1]

To a solution of racemic-tert-butyl ((1R,2R,4S,6S)-6-hydroxybicyclo[2.2.1]heptan-2-yl)carbamate (330 mg, 1.45 mmol) in THF (40 mL) was added KOtBu (326 mg, 2.90 mmol) at 10° C., the reaction was stirred for 10 mins. 4-Chloro-6-(1-methylpyrazol-4-yl)pyrazolo[1,5-a]pyrazine (226 mg, 968 μmol) was added and the reaction was stirred at 10° C. for 5 h. Water (30 mL) was added and the mixture was extracted with DCM (3×100 mL). The combined organics were washed with brine (50 mL), then dried over Na₂SO₄, filtered and concentrated under vacuum to give racemic-tert-butyl ((1R,2R,4S,6S)-6-((6-(1-methyl-1H-pyrazol-4-yl)pyrazolo[1,5-a]pyrazin-4-yl)oxy)bicyclo[2.2.1]heptan-2-yl)carbamate (320 mg, 78% yield) as yellow oil. LCMS m/z=425.3 (M+H)+

### 6. Synthesis of racemic-tert-butyl methyl((1R,2R,4S,6S)-6-((6-(1-methyl-1H-pyrazol-4-yl)pyrazolo[1,5-a]pyrazin-4-yl)oxy)bicyclo[2.2.1]heptan-2-yl)carbamate

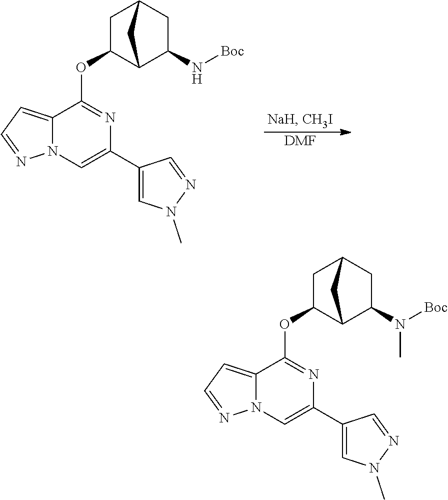

Iodomethane (127 mg, 895 μmol) was added dropwise to a solution of racemic-tert-butyl ((1R,2R,4S,6S)-6-((6-(1-methyl-1H-pyrazol-4-yl)pyrazolo[1,5-a]pyrazin-4-yl)oxy)bicyclo[2.2.1]heptan-2-yl)carbamate (190 mg, 448 μmol) in DMF (10 mL). NaH (21 mg, 895 μmol) was added and the reaction stirred at 15° C. for 2 h. The mixture was quenched with H₂O (0.5 mL), filtered and the filtrate concentrated under vacuum and the crude product was purified by prep-TLC (PE/EtOAc=3/1) to give racemic-tert-butyl methyl((1R,2R,4S,6S)-6-((6-(1-methyl-1H-pyrazol-4-yl)pyrazolo[1,5-a]pyrazin-4-yl)oxy)bicyclo[2.2.1]heptan-2-yl)carbamate (160 mg, 68% yield) as yellow oil. LCMS m/z=439.3 (M+H)+

### 7. Synthesis of racemic-(1R,2R,4S,6S)—N-methyl-6-((6-(1-methyl-1H-pyrazol-4-yl)pyrazolo[1,5-a]pyrazin-4-yl)oxy)bicyclo[2.2.1]heptan-2-amine hydrochloride

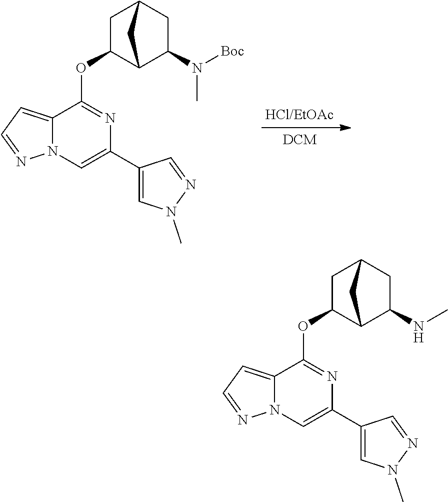 [SMILES: CN(C(=O)OC(C)(C)C)[C@@H]1C[C@@H]2C[C@H]1[C@@H](Oc1nc(-c3cnn(C)c3)cn3nccc13)C2 ; CN[C@@H]1C[C@@H]2C[C@H]1[C@@H](Oc1nc(-c3cnn(C)c3)cn3nccc13)C2]

A solution of racemic-tert-butyl methyl((1R,2R,4S,6S)-6-((6-(1-methyl-1H-pyrazol-4-yl)pyrazolo[1,5-a]pyrazin-4-yl)oxy)bicyclo[2.2.1]heptan-2-yl)carbamate (160 mg, 365 μmol) in HCl/EtOAc (10 mL) and DCM (20 mL) was stirred at 15° C. for 1 h. The mixture was filtered and concentrated under vacuum to give racemic-(1R,2R,4S,6S)—N-methyl-6-((6-(1-methyl-1H-pyrazol-4-yl)pyrazolo[1,5-a]pyrazin-4-yl)oxy)bicyclo[2.2.1]heptan-2-amine hydrochloride (130 mg, crude) as yellow solid. LCMS m/z=339.2 (M+H)+

### 8. Synthesis of racemic-N-methyl-N—((1R,2R,4S,6S)-6-((6-(1-methyl-1H-pyrazol-4-yl)pyrazolo[1,5-a]pyrazin-4-yl)oxy)bicyclo[2.2.1]heptan-2-yl)but-2-ynamide

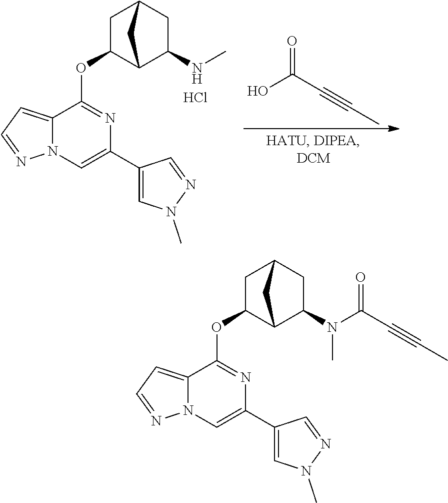

To a solution of racemic-(1R,2R,4S,6S)—N-methyl-6-((6-(1-methyl-1H-pyrazol-4-yl)pyrazolo[1,5-a]pyrazin-4-yl)oxy)bicyclo[2.2.1]heptan-2-amine hydrochloride (120 mg, 355 μmol) in DCM (30 mL) was added DIPEA (92 mg, 709 μmol) then but-2-ynoic acid (60 mg, 709 μmol) was added and the mixture stirred at 15° C. for 5 min. HATU (135 mg, 355 μmol) was added and the reaction stirred at 15° C. for 1 h. The mixture was filtered and the filtrate concentrated under vacuum. The residue was purified by column chromatography column on silica gel (PE/EtOAc=1/0 to 1/3) to give racemic-N-methyl-N-((1R,2R,4S,6S)-6-((6-(1-methyl-1H-pyrazol-4-yl)pyrazolo[1,5-a]pyrazin-4-yl)oxy)bicyclo[2.2.1]heptan-2-yl)but-2-ynamide (110 mg, 66% yield) as yellow oil. LCMS m/z=405.3 (M+H)+

### 9. Synthesis of N-methyl-N-((1S,2S,4R,6R) and (1R,2R,4S,6S)-6-((6-(1-methyl-1H-pyrazol-4-yl)pyrazolo[1,5-a]pyrazin-4-yl)oxy)bicyclo[2.2.1]heptan-2-yl)but-2-ynamide

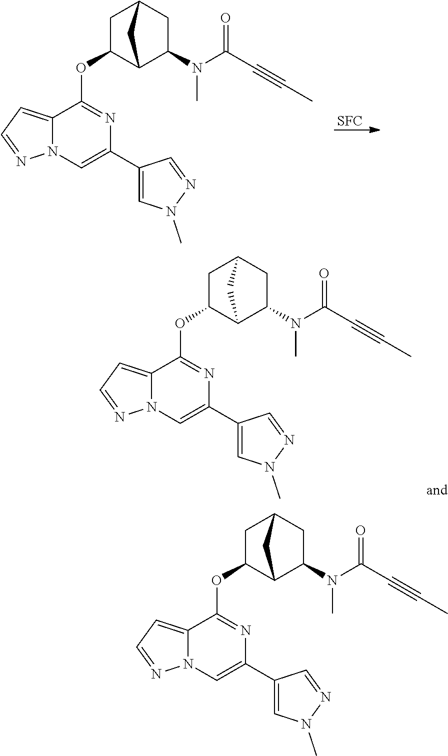

Racemic-N-methyl-N-((1R,2R,4S,6S)-6-((6-(1-methyl-1H-pyrazol-4-yl)pyrazolo[1,5-a]pyrazin-4-yl)oxy)bicyclo[2.2.1]heptan-2-yl)but-2-ynamide (120 mg, 297 μmol) was purified by SFC (column: DAICEL CHIRALCEL OJ-H (250 mm*30 mm, 5 um), 30%[0.1% NH₃H₂O, EtOH] as mobile phase, Flow Rate (mL/min): 60) to give

the first eluting diastereosiomer, Peak 1, (50 mg, 39% yield) as white solid. LCMS m/z=405.1 (M+H)+. 1H NMR (400 MHz, DMSO-d₆) δ=8.76-8.74 (m, 1H), 8.23-8.16 (m, 1H), 8.03-7.99 (m, 2H), 6.81-6.78 (m, 1H), 5.22-5.15 (m, 1H), 4.50-4.46 (m, 1H), 3.89 (s, 3H), 3.11-2.79 (m, 3H), 2.60-2.57 (m, 1H), 2.43 (s, 1H), 2.10-1.96 (m, 4H), 1.66-1.54 (m, 5H).

the second eluting diastereoisomer, Peak 2 (42 mg, 34% yield) as white solid. LCMS m/z=405.1 (M+H)+. 1H NMR: (400 MHz, DMSO-d₆) δ=8.77-8.75 (m, 1H), 8.24-8.17 (m, 1H), 8.03-7.99 (m, 2H), 6.82-6.78 (m, 1H), 5.22-5.15 (m, 1H), 4.54-4.46 (m, 1H), 3.89 (s, 3H), 3.12-2.80 (m, 3H), 2.61-2.57 (m, 1H), 2.43 (s, 1H), 2.10-1.98 (m, 4H), 1.66-1.64 (m, 5H).

The absolute stereochemistry of each product in each peak was not assigned.

### Example 124. 1-(1-methyl-4-((6-(1-methyl-1H-pyrazol-4-yl)pyrazolo[1,5-a]pyrazin-4-yl)oxy)-2-azabicyclo[2.1.1]hexan-2-yl)prop-2-en-1-one

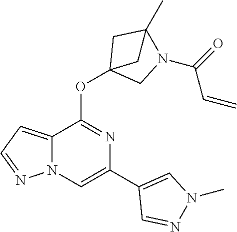 [SMILES: C=CC(=O)N1CC2(Oc3nc(-c4cnn(C)c4)cn4nccc34)CC1(C)C2]

### 1. Synthesis of tert-butyl 1-methyl-4-((6-(1-methyl-1H-pyrazol-4-yl)pyrazolo[1,5-a]pyrazin-4-yl)oxy)-2-azabicyclo[2.1.1]hexane-2-carboxylate

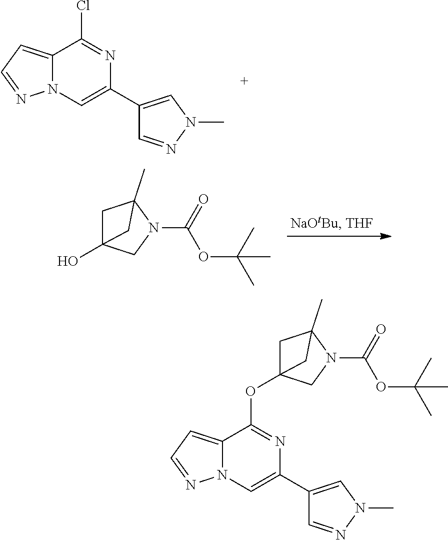

NaOtBu (202 mg, 2.11 mmol) was added carefully in portions to an ice-cold solution of tert-butyl 4-hydroxy-1-methyl-2-azabicyclo[2.1.1]hexane-2-carboxylate (284 mg, 1.34 mmol) in anhydrous THF (10 mL). After 10 mins, 4-chloro-6-(1-methyl-1H-pyrazol-4-yl)pyrazolo[1,5-a]pyrazine (Intermediate A, 300 mg, 1.28 mmol) was added in portions to the cold mixture. The reaction mixture was allowed to warm to 23° C. and stirring continued for 18 h. The reaction mixture was diluted with EtOAc and filtered through Celite®. The filtrate was evaporated to dryness in vacuo and the residue purified by column chromatography (20-65% EtOAc/heptane) to give tert-butyl 1-methyl-4-((6-(1-methyl-1H-pyrazol-4-yl)pyrazolo[1,5-a]pyrazin-4-yl)oxy)-2-azabicyclo[2.1.1]hexane-2-carboxylate (161 mg, 31% yield). LCMS m/z=411.2 (M+H)+

### 2. Synthesis of 1-methyl-4-((6-(1-methyl-1H-pyrazol-4-yl)pyrazolo[1,5-a]pyrazin-4-yl)oxy)-2-azabicyclo[2.1.1]hexane

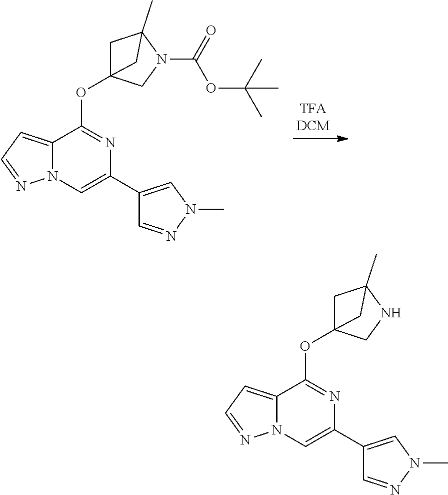 [SMILES: Cn1cc(-c2cn3nccc3c(OC34CN(C(=O)OC(C)(C)C)C(C)(C3)C4)n2)cn1 ; Cn1cc(-c2cn3nccc3c(OC34CNC(C)(C3)C4)n2)cn1]

TFA (1.49 g, 13.1 mmol) was added to a solution of tert-butyl 1-methyl-4-((6-(1-methyl-1H-pyrazol-4-yl)pyrazolo[1,5-a]pyrazin-4-yl)oxy)-2-azabicyclo[2.1.1]hexane-2-carboxylate (161 mg, 392 μmol) in DCM (3 mL) and the mixture stirred for 1 h. The reaction mixture was evaporated to dryness in vacuo to afford 1-methyl-4-((6-(1-methyl-1H-pyrazol-4-yl)pyrazolo[1,5-a]pyrazin-4-yl)oxy)-2-azabicyclo[2.1.1]hexane which was used without further purification. LCMS m/z=311.1 (M+H)+

### 3. Synthesis of 1-(1-methyl-4-((6-(1-methyl-1H-pyrazol-4-yl)pyrazolo[1,5-a]pyrazin-4-yl)oxy)-2-azabicyclo[2.1.1]hexan-2-yl)prop-2-en-1-one

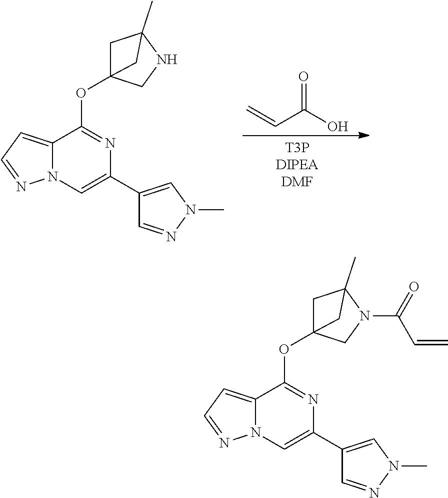

TEA (239 mg, 2.37 mmol) and acryloyl chloride (128 mg, 1.42 mmol) were added sequentially to a solution of 1-methyl-4-((6-(1-methyl-1H-pyrazol-4-yl)pyrazolo[1,5-a]pyrazin-4-yl)oxy)-2-azabicyclo[2.1.1]hexane in DCM (4 mL) at 0° C. and the mixture stirred for 3 min. The reaction was quenched with sat. aq. NaHCO₃ and extracted into DCM. The combined extracts were dried (Na₂SO₄) and evaporated to dryness in vacuo and the residue purified by column chromatography (0-10% MeOH/EtOAc) to afford 1-(1-methyl-4-((6-(1-methyl-1H-pyrazol-4-yl)pyrazolo[1,5-a]pyrazin-4-yl)oxy)-2-azabicyclo[2.1.1]hexan-2-yl)prop-2-en-1-one (44 mg, 10% yield). LCMS m/z=365.2 (M+H)⁺; ¹H NMR (500 MHz, CDCl₃) δ 8.27 (s, 1H), 7.92 (d, J=2.26 Hz, 1H), 7.81 (s, 1H), 7.66 (s, 1H), 6.75 (d, J=2.26 Hz, 1H), 6.41 (br d, J=5.02 Hz, 2H), 5.67-5.72 (m, 1H), 3.98 (s, 5H), 2.44-2.51 (m, 2H), 2.20-2.27 (m, 2H), 2.00-2.08 (m, 3H).

### Example 125. N-(4-((6-(1-methyl-1H-pyrazol-4-yl)pyrazolo[1,5-a]pyrazin-4-yl)oxy)bicyclo[2.2.1]heptan-1-yl)acrylamide

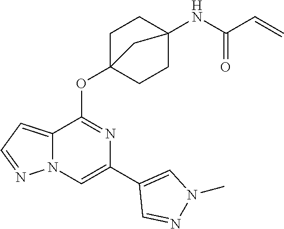 [SMILES: C=CC(=O)NC12CCC(Oc3nc(-c4cnn(C)c4)cn4nccc34)(CC1)C2]

N-(4-((6-(1-Methyl-1H-pyrazol-4-yl)pyrazolo[1,5-a]pyrazin-4-yl)oxy)bicyclo[2.2.1]heptan-1-yl)acrylamide was prepared as a white solid (34.4 mg, 7% yield, 95% purity) from 4-chloro-6-(1-methyl-1H-pyrazol-4-yl)pyrazolo[1,5-a]pyrazine (Intermediate A) and tert-butyl (4-hydroxybicyclo[2.2.1]heptan-1-yl)carbamate using an analogous 3-part procedure as described for Example 124. LCMS m/z=379.2 (M+H)⁺; ¹H NMR (400 MHz, CDCl₃) δ 8.21 (d, J=1.00 Hz, 1H), 7.88 (d, J=2.26 Hz, 1H), 7.86-7.87 (m, 1H), 7.80 (s, 1H), 6.72 (dd, J=1.00, 2.26 Hz, 1H), 6.28-6.33 (m, 1H), 6.05-6.14 (m, 1H), 5.70-5.74 (m, 1H), 5.63-5.68 (m, 1H), 4.01 (s, 3H), 2.54-2.58 (m, 2H), 2.43-2.51 (m, 2H), 2.27 (s, 4H), 2.02-2.10 (m, 2H).

### Example 126. 1-(6-(((6-(1-methyl-1H-pyrazol-4-yl)pyrazolo[1,5-a]pyrazin-4-yl)oxy)methyl)-3-azabicyclo[3.1.1]heptan-3-yl)prop-2-en-1-one

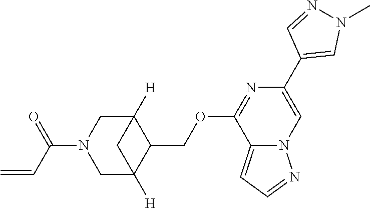 [SMILES: C=CC(=O)N1CC2CC(C1)C2COc1nc(-c2cnn(C)c2)cn2nccc12]

### 1. Synthesis of tert-butyl 6-(((6-(1-methyl-1H-pyrazol-4-yl)pyrazolo[1,5-a]pyrazin-4-yl)oxy)methyl)-3-azabicyclo[3.1.1]heptane-3-carboxylate

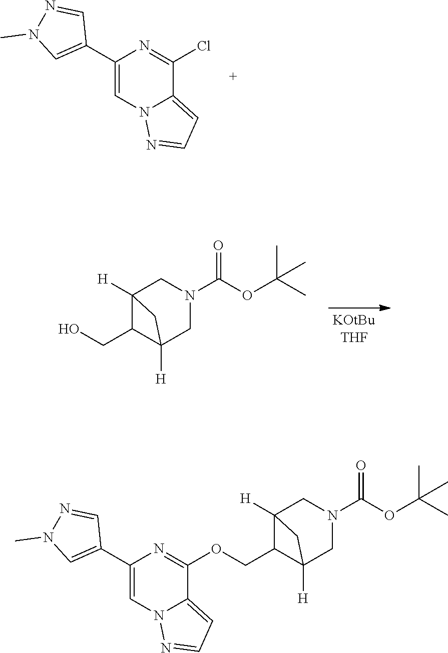 [SMILES: Cn1cc(-c2cn3nccc3c(Cl)n2)cn1 ; CC(C)(C)OC(=O)N1CC2CC(C1)C2CO ; Cn1cc(-c2cn3nccc3c(OCC3C4CC3CN(C(=O)OC(C)(C)C)C4)n2)cn1]

KOtBu (36 mg, 0.321 mmol, 1 M solution) was added to a solution of tert-butyl 6-(hydroxymethyl)-3-azabicyclo[3.1.1]heptane-3-carboxylate (55 mg, 0.257 mmol) in THF (1 mL) and the mixture stirred at rt for 5 min. 4-Chloro-6-(1-methyl-1H-pyrazol-4-yl)pyrazolo[1,5-a]pyrazine (Intermediate A, 50 mg, 0.214 mmol) was added and stirring continued for 10 min. The reaction mixture was evaporated to dryness to afford tert-butyl 6-(((6-(1-methyl-1H-pyrazol-4-yl)pyrazolo[1,5-a]pyrazin-4-yl)oxy)methyl)-3-azabicyclo[3.1.1]heptane-3-carboxylate and used without further purification. LCMS m/z=447.5 (M+Na)+;

### 2. 1-(6-(((6-(1-methyl-1H-pyrazol-4-yl)pyrazolo[1,5-a]pyrazin-4-yl)oxy)methyl)-3-azabicyclo[3.1.1]heptan-3-yl)prop-2-en-1-one

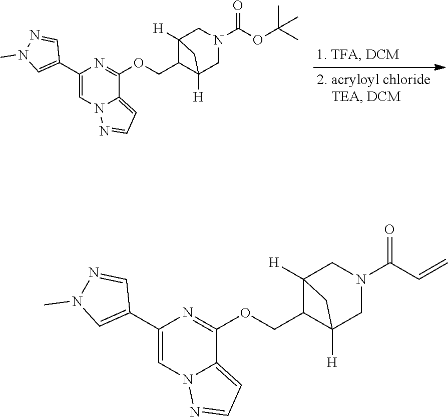

tert-butyl 6-(((6-(1-methyl-1H-pyrazol-4-yl)pyrazolo[1,5-a]pyrazin-4-yl)oxy)methyl)-3-azabicyclo[3.1.1]heptane-3-carboxylate (91 mg, 0.214 mmol) was dissolved in DCM (1.5 mL) and TFA (164 μL, 2.14 mmol) was added and the reaction stirred for 2 h. The reaction was loaded onto an SCX ion exchange column (pre-wetted with MeOH) and the column washed with MeOH. The product was liberated using 7M NH₃/MeOH solution and the combined organics evaporated to dryness in vacuo. The residue was dissolved in DCM (1.5 mL) and TEA (65 mg, 0.642 mmol) added, the solution cooled to −78° C. and acryloyl chloride added and the mixture stirred for 10 min. The reaction mixture was evaporated to dryness in vacuo and the residue purified by prep-HPLC (Waters XSelect CSH Prep C18 5 μm OBD 19×100 mm, 5-55% MeCN:H₂O w/0.1% NH₄OH modifier) to afford 1-(6-(((6-(1-methyl-1H-pyrazol-4-yl)pyrazolo[1,5-a]pyrazin-4-yl)oxy)methyl)-3-azabicyclo[3.1.1]heptan-3-yl)prop-2-en-1-one (7 mg). LCMS m/z=379.3 (M+H)⁺; ¹H NMR (500 MHz, DMSO-d₆) δ 8.76 (d, J=1.2 Hz, 1H), 8.23 (s, 1H), 8.03 (s, 1H), 8.01 (d, J=2.4 Hz, 1H), 6.86-6.82 (m, 1H), 6.73 (dd, J=10.4, 17.1 Hz, 1H), 6.17 (dd, J=2.4, 16.5 Hz, 1H), 5.69 (dd, J=3.1, 10.4 Hz, 1H), 4.86 (d, J=7.9 Hz, 2H), 3.91 (d, J=2.4 Hz, 1H), 3.89 (s, 3H), 3.86-3.82 (m, 1H), 3.71 (dd, J=2.1, 13.1 Hz, 1H), 3.64-3.58 (m, 1H), 2.61-2.55 (m, 1H), 2.45 (dd, J=2.4, 6.1 Hz, 2H), 2.22 (dt, J=5.5, 7.9 Hz, 1H), 1.38 (dd, J=5.5, 9.8 Hz, 1H).

### Examples 127 & 128. 1-((1R,5S,6r)-6-(((6-(1-methyl-1H-pyrazol-4-yl)pyrazolo[1,5-a]pyrazin-4-yl)oxy)methyl)-3-azabicyclo[3.1.1]heptan-3-yl)prop-2-en-1-one and 1-((1R,5S,6s)-6-(((6-(1-methyl-1H-pyrazol-4-yl)pyrazolo[1,5-a]pyrazin-4-yl)oxy)methyl)-3-azabicyclo[3.1.1]heptan-3-yl)prop-2-en-1-one

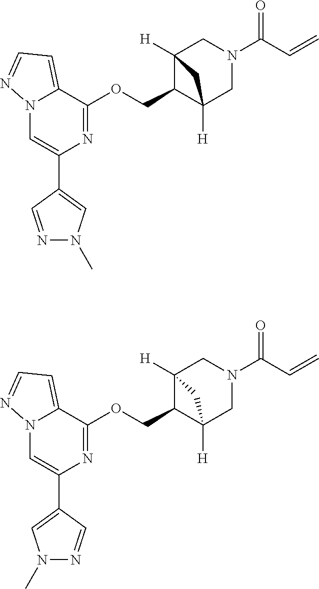 [SMILES: C=CC(=O)N1C[C@H]2C[C@@H](C1)[C@@H]2COc1nc(-c2cnn(C)c2)cn2nccc12 ; C=CC(=O)N1C[C@H]2C[C@@H](C1)[C@H]2COc1nc(-c2cnn(C)c2)cn2nccc12]

A vial was charged with tert-butyl (1S,5R)-6-(hydroxymethyl)-3-azabicyclo[3.1.1]heptane-3-carboxylate (0.07 g, 0.308 mmol) and THF (1.23 mL). KOtBu (1.0 M, 462 μL) was added the reaction stirred for 5 mins, 4-chloro-6-(1-methylpyrazol-4-yl)pyrazolo[1,5-a]pyrazine (72 mg, 0.308 mmol) was then added and the reaction was stirred for 15 mins. The reaction was concentrated to afford an orange solid. The solid was dissolved in HCl (1.25 M, 4.93 mL) and stirred at 50° C. overnight. The reaction was concentrated, dissolved in DCM (1.5 mL), and TEA (312 mg, 3.08 mmol) was added. The reaction was cooled to −78° C. in a dry ice/acetone bath and acryloyl chloride (42 mg, 0.462 mmol) was added and the reaction was stirred for 15 mins at −78° C. The reaction was loaded directly onto a silica cartridge and purified by column chromatography (12 g silica column, gradient elution 0-100% (3:1 EtOAc:EtOH):Heptane) to afford 62 mg of a diastereomeric mixture.

The material was purified by prep SFC (CHIRALPAK IA 30×250 mm, 5 um, Method: 40% MeOH w/No Modifier in CO₂ (flow rate: 100 mL/min, ABPR 120 bar, MBPR 40 psi, column temp 40° C. to provide: Received 12 mg of Peak E1 and 14 mg of Peak E2.

### Example 127: Diastereoisomer 1, Peak E1, 12 mg, 10% yield. LCMS m/z=379.1 (M+H)⁺; 1H-NMR (500 MHz, DMSO-d₆) δ=8.74 (s, 1H), 8.15 (s, 1H), 8.01 (d, J=2.4 Hz, 1H), 7.99 (s, 1H), 6.82 (d, J=2.4 Hz, 1H), 6.77 (dd, J=10.4, 17.1 Hz, 1H), 6.20 (br d, J=14.7 Hz, 1H), 5.75-5.65 (m, 1H), 4.69-4.60 (m, 1H), 4.50 (dd, J=7.3, 11.6 Hz, 1H), 3.93-3.82 (m, 5H), 3.76-3.60 (m, 2H), 2.76 (s, 1H), 2.58 (br s, 2H), 2.07 (s, 2H), 1.36 (d, J=9.2 Hz, 1H)

### Example 128: Diastereoisomer 2, Peak E2, (14 mg, 12% yield. LCMS m/z=379.1 (M+H)⁺. 1H NMR (500 MHz, DMSO-d₆) δ=8.76 (d, J=1.2 Hz, 1H), 8.23 (s, 1H), 8.03 (s, 1H), 8.02 (d, J=2.4 Hz, 1H), 6.86-6.82 (m, 1H), 6.73 (dd, J=10.4, 17.1 Hz, 1H), 6.17 (dd, J=2.4, 16.5 Hz, 1H), 5.72-5.66 (m, 1H), 4.86 (d, J=7.9 Hz, 2H), 3.96-3.83 (m, 5H), 3.73-3.58 (m, 2H), 2.59-2.52 (m, 2H), 2.45 (dd, J=1.8, 6.7 Hz, 2H), 2.25-2.18 (m, 1H), 1.37 (dd, J=5.5, 9.8 Hz, 1H)

### Example 129. trans-N-methyl-N-(2-(((6-(1-methyl-1H-pyrazol-4-yl)pyrazolo[1,5-a]pyrazin-4-yl)oxy)methyl)cyclobutyl)acrylamide

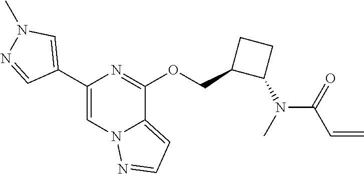 [SMILES: C=CC(=O)N(C)[C@H]1CC[C@@H]1COc1nc(-c2cnn(C)c2)cn2nccc12]

### 1. Synthesis of trans-tert-butyl (2-(hydroxymethyl)cyclobutyl)carbamate

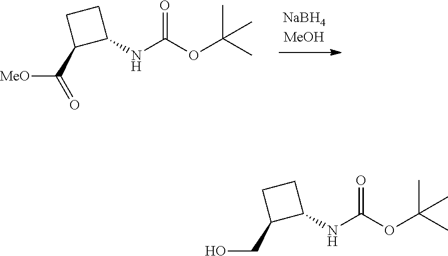 [SMILES: COC(=O)[C@H]1CC[C@@H]1NC(=O)OC(C)(C)C ; CC(C)(C)OC(=O)N[C@H]1CC[C@@H]1CO]

NaBH₄ (206 mg, 5.45 mmol) was added to a solution of trans-methyl-2-((tert-butoxycarbonyl)amino)cyclobutane-1-carboxylate (250 mg, 1.09 mmol) in MeOH (11 mL) and the mixture stirred at rt for 4 h. Additional NaBH₄ (206 mg, 5.45 mmol) was added and the reaction stirred overnight at rt. Additional NaBH₄ (206 mg, 5.45 mmol) was added and the reaction was stirred for 8 h. The reaction was quenched with sat. NH₄Cl solution, diluted with H₂O and extracted with EtOAc. The combined organics were dried (Na₂SO₄) and evaporated to dryness in vacuo to afford trans-tert-butyl N-(2-(hydroxymethyl)cyclobutyl)carbamate as a white solid (199 mg, 91% yield). ¹H NMR (500 MHz, CDCl₃) δ 4.84 (br s, 1H), 3.66-3.59 (m, 1H), 3.59-3.48 (m, 2H), 2.39-2.28 (m, 1H), 2.26-2.18 (m, 1H), 1.82 (q, J=9.8 Hz, 1H), 1.73 (quin, J=9.9 Hz, 1H), 1.45 (s, 9H), 1.42-1.30 (m, 1H)

### 2. Synthesis of trans-tert-butyl methyl(2-(((6-(1-methyl-1H-pyrazol-4-yl)pyrazolo[1,5-a]pyrazin-4-yl)oxy)methyl)cyclobutyl)carbamate

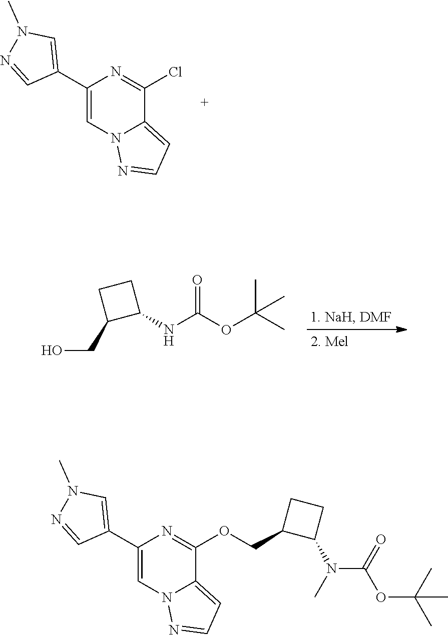 [SMILES: Cn1cc(-c2cn3nccc3c(Cl)n2)cn1 ; CC(C)(C)OC(=O)N[C@H]1CC[C@@H]1CO ; CN(C(=O)OC(C)(C)C)[C@H]1CC[C@@H]1COc1nc(-c2cnn(C)c2)cn2nccc12]

NaH (38.5 mg, 0.963 mmol, 60% purity) was added to solution of 4-chloro-6-(1-methylpyrazol-4-yl)pyrazolo[1,5-a]pyrazine (75 mg, 0.322 mmol) and tert-butyl N-(2-(hydroxymethyl)cyclobutyl)carbamate (0.385 mmol) in DMF (1.6 mL) and the mixture was stirred at RT for 10 min. Iodomethane (137 mg, 0.963 mmol) was added and the reaction mixture stirred overnight at RT. The reaction was quenched with saturated ammonium chloride solution and extracted with EtOAc (2×). The combined organics were washed with brine, dried (Na₂SO₄) and evaporated to dryness in vacuo. The residue was purified using column chromatography (SiO₂, 0-75% [3:1 EtOAc:EtOH]:Heptane) to afford trans-tert-butyl methyl(2-(((6-(1-methyl-1H-pyrazol-4-yl)pyrazolo[1,5-a]pyrazin-4-yl)oxy)methyl)cyclobutyl)carbamate as a clear colourless oil (91 mg, 68% yield). LCMS m/z=413.2 (M+H)+

### 3. Synthesis of trans-N-methyl-N-(2-(((6-(1-methyl-1H-pyrazol-4-yl)pyrazolo[1,5-a]pyrazin-4-yl)oxy)methyl)cyclobutyl)acrylamide

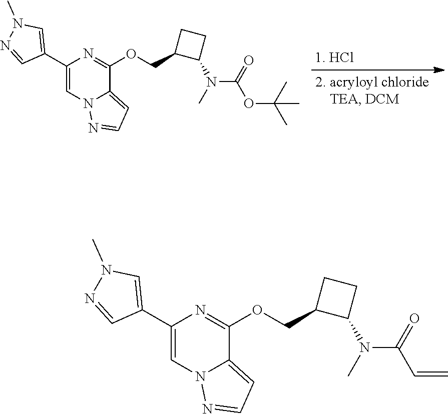 [SMILES: CN(C(=O)OC(C)(C)C)[C@H]1CC[C@@H]1COc1nc(-c2cnn(C)c2)cn2nccc12 ; C=CC(=O)N(C)[C@H]1CC[C@@H]1COc1nc(-c2cnn(C)c2)cn2nccc12]

Trans-tert-butyl methyl(2-(((6-(1-methyl-1H-pyrazol-4-yl)pyrazolo[1,5-a]pyrazin-4-yl)oxy)methyl)cyclobutyl)carbamate (91 mg, 0.221 mmol) and HCl (1.25 M, 1.76 mL) was heated to 50° C. and stirred for 8 h. The reaction mixture was evaporated to dryness in vacuo and the residue dissolved in DCM (2 mL), TEA (223 mg, 2.21 mmol) added and the mixture cooled to −78° C. Acryloyl chloride (22 mg, 0.243 mmol) was added and the mixture stirred at −78° C. for 15 min. The reaction mixture was loaded onto a SiO₂ cartridge and purified by column chromatography (0-100% [3:1 EtOAc:EtOH]:Heptane) to afford trans N-methyl-N—(-2-(((6-(1-methyl-1H-pyrazol-4-yl)pyrazolo[1,5-a]pyrazin-4-yl)oxy)methyl)cyclobutyl)acrylamide as an off-white solid (49 mg, 61% yield). LCMS m/z=367.1 (M+H)⁺; ¹H NMR (500 MHz, DMSO-d₆) δ 8.73 (s, 1H), 8.17 (s, 1H), 8.03-7.96 (m, 2H), 6.81-6.64 (m, 2H), 6.07-5.90 (m, 1H), 5.67-5.44 (m, 1H), 4.86 (br s, 1H), 4.67-4.44 (m, 3H), 3.88 (s, 3H), 3.09 (br s, 1H), 3.03-2.85 (m, 3H), 2.20-1.95 (m, 2H), 1.93-1.81 (m, 1H), 1.57 (br s, 1H).

### Example 130. N-(4-fluoro-3-((6-(1-methyl-1H-pyrazol-4-yl)pyrazolo[1,5-a]pyrazin-4-yl)oxy)phenyl)-N-methylacrylamide

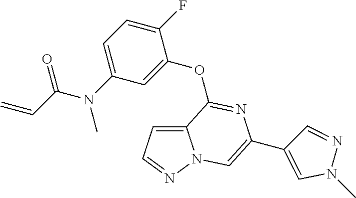 [SMILES: C=CC(=O)N(C)c1ccc(F)c(Oc2nc(-c3cnn(C)c3)cn3nccc23)c1]

### 1. Synthesis of 4-fluoro-3-((6-(1-methyl-1H-pyrazol-4-yl)pyrazolo[1,5-a]pyrazin-4-yl)oxy)aniline

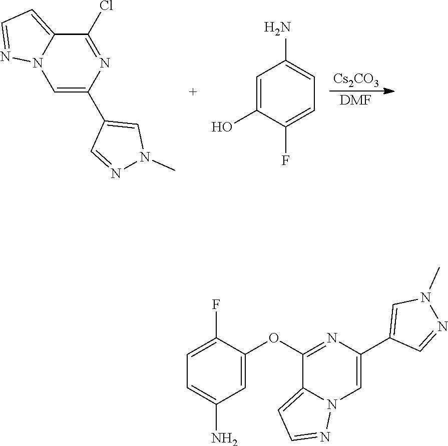

Cs₂CO₃ (139 mg, 0.428 mmol) was added to a mixture of 4-chloro-6-(1-methyl-1H-pyrazol-4-yl)pyrazolo[1,5-a]pyrazine (Intermediate A, 100 mg, 0.428 mmol) and 5-amino-2-fluoro-phenol (54.4 mg, 0.428 mmol) in dry DMF (1 mL) in a microwave vial which was capped and heated in a MW oven at 110° C. for 10 min. After cooling to RT the mixture was diluted with water and extracted with EtOAc (2×). The combined organics were washed with brine, dried (Na₂SO₄) and evaporated to dryness in vacuo. The residue was purified by column chromatography (100% EtOAc) to give 4-fluoro-3-((6-(1-methyl-1H-pyrazol-4-yl)pyrazolo[1,5-a]pyrazin-4-yl)oxy)aniline as a brown solid (120 mg, 78% yield, 90% purity). LCMS m/z=325.3 (M+H)+;

### 2. Synthesis of N-(4-fluoro-3-((6-(1-methyl-1H-pyrazol-4-yl)pyrazolo[1,5-a]pyrazin-4-yl)oxy)phenyl)acrylamide

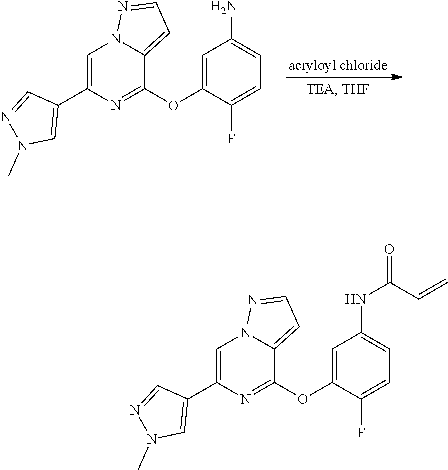 [SMILES: Cn1cc(-c2cn3nccc3c(Oc3cc(N)ccc3F)n2)cn1 ; "acryloyl chloride" ; C=CC(=O)Nc1ccc(F)c(Oc2nc(-c3cnn(C)c3)cn3nccc23)c1]

A screw top vial was charged with 4-fluoro-3-((6-(1-methyl-1H-pyrazol-4-yl)pyrazolo[1,5-a]pyrazin-4-yl)oxy)aniline (88 mg, 0.271 mmol) and THF (2 mL). To this was added acryloyl chloride (36.8 mg, 0.407 mmol) was added, followed by TEA (41.2 mg, 0.407 mmol) with stirring at RT for 3 h. The reaction mixture was evaporated to dryness in vacuo and the residue purified by column chromatography (heptane/EtOAc=1/1) to give N-(4-fluoro-3-((6-(1-methyl-1H-pyrazol-4-yl)pyrazolo[1,5-a]pyrazin-4-yl)oxy)phenyl)acrylamide as a white solid (97 mg, 90% yield, 95% purity). LCMS m/z=379.1 (M+H)+. ¹H NMR (400 MHz, DMSO-d₆) δ 10.45-10.32 (m, 1H), 9.03-8.84 (m, 1H), 8.23-8.15 (m, 1H), 8.05-7.93 (m, 1H), 7.92-7.85 (m, 1H), 7.83-7.74 (m, 1H), 7.62-7.51 (m, 1H), 7.50-7.39 (m, 1H), 7.08 (br dd, J=2.1, 4.4 Hz, 1H), 6.49-6.38 (m, 1H), 6.36-6.22 (m, 1H), 5.87-5.74 (m, 1H), 3.81 (s, 3H).

### 3. Synthesis of N-(4-fluoro-3-((6-(1-methyl-1H-pyrazol-4-yl)pyrazolo[1,5-a]pyrazin-4-yl)oxy)phenyl)-N-methylacrylamide

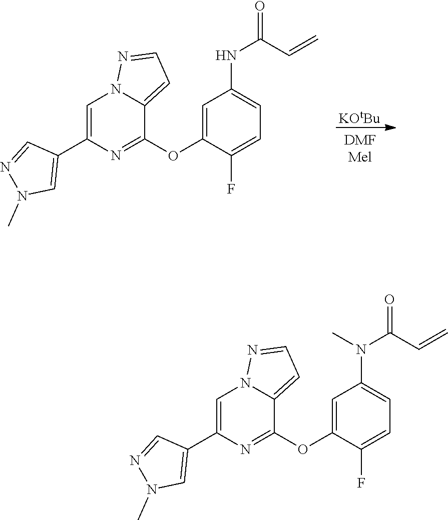 [SMILES: C=CC(=O)Nc1ccc(F)c(Oc2nc(-c3cnn(C)c3)cn3nccc23)c1 ; C=CC(=O)N(C)c1ccc(F)c(Oc2nc(-c3cnn(C)c3)cn3nccc23)c1 ; "KOtBu"]

To a mixture of N-(4-fluoro-3-((6-(1-methyl-1H-pyrazol-4-yl)pyrazolo[1,5-a]pyrazin-4-yl)oxy)phenyl)acrylamide (60 mg, 0.159 mmol) in DMF (1.5 mL) was added iodomethane (67.5 mg, 0.476 mmol). To this was added KO^{t}Bu (1 M soln in THF, 0.444 mL) dropwise with stirring at RT. The mixture was diluted with EtOAc and washed with water. The combined organics were dried (Na₂SO₄) and evaporated to dryness in vacuo. The residue was purified by prep HPLC (Waters XSelect CSH Prep C18 5 μm OBD 19×100 mm; Gradient: 5-70% MeCN:H₂O w/0.1% NH₄OH modifier) to afford N-(4-fluoro-3-((6-(1-methyl-1H-pyrazol-4-yl)pyrazolo[1,5-a]pyrazin-4-yl)oxy)phenyl)-N-methylacrylamide as a white solid (18 mg, 27% yield, 95% purity). LCMS m/z=393.4 (M+H)+; ¹H NMR (400 MHz, DMSO-d₆) δ 9.01-8.90 (m, 1H), 8.24-8.14 (m, 1H), 7.88 (s, 1H), 7.75 (d, J=0.8 Hz, 1H), 7.62 (dd, J=2.5, 7.0 Hz, 1H), 7.61-7.50 (m, 1H), 7.43-7.27 (m, 1H), 7.16-7.07 (m, 1H), 6.27-6.12 (m, 2H), 5.63-5.50 (m, 1H), 3.81 (s, 3H), 3.29-3.23 (m, 3H)

### Example 131. (E)-N-(2-methyl-5-((6-(1-methyl-1H-pyrazol-4-yl)pyrazolo[1,5-a]pyrazin-4-yl)oxy)phenyl)but-2-enamide

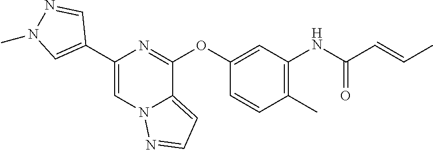 [SMILES: C/C=C/C(=O)Nc1cc(Oc2nc(-c3cnn(C)c3)cn3nccc23)ccc1C]

### 1. Synthesis of 2-methyl-5-((6-(1-methyl-1H-pyrazol-4-yl)pyrazolo[1,5-a]pyrazin-4-yl)oxy)aniline

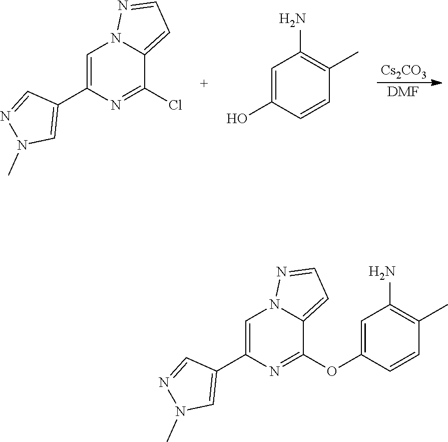

Cs₂CO₃ (293 mg, 0.899 mmol) was added to a mixture of 4-chloro-6-(1-methyl-1H-pyrazol-4-yl)pyrazolo[1,5-a]pyrazine (Intermediate A, 210 mg, 0.899 mmol) and 3-amino-4-methylphenol (III mg, 0.899 mmol) in dry DMF (3 mL) in a microwave vial which was capped and heated in a MW oven at 110° C. for 10 min. After cooling to RT the mixture was diluted with water and extracted with EtOAc (2×). The combined organics were washed with brine, dried (Na₂SO₄) and evaporated to dryness in vacuo. The residue was purified by column chromatography (100% EtOAc) to give 2-methyl-5-((6-(1-methyl-1H-pyrazol-4-yl)pyrazolo[1,5-a]pyrazin-4-yl)oxy)aniline as an off-white solid (250 mg, 78% yield, 90% purity). ¹H NMR (500 MHz, DMSO-d₆) δ: 8.88-8.83 (m, 1H), 8.11-8.07 (m, 1H), 7.93-7.91 (m, 1H), 7.86-7.83 (m, 1H), 7.00-6.97 (m, 1H), 6.93-6.88 (m, 1H), 6.59-6.56 (m, 1H), 6.46-6.40 (m, 1H), 5.07-5.01 (m, 2H), 3.85-3.82 (m, 3H), 2.11-2.05 (m, 3H).

### 2. Synthesis of (E)-N-(2-methyl-5-((6-(1-methyl-1H-pyrazol-4-yl)pyrazolo[1,5-a]pyrazin-4-yl)oxy)phenyl)but-2-enamide

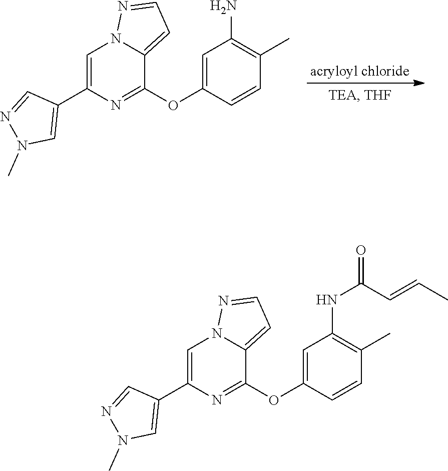 [SMILES: Cc1ccc(Oc2nc(-c3cnn(C)c3)cn3nccc23)cc1N ; "acryloyl chloride" ; C/C=C/C(=O)Nc1cc(Oc2nc(-c3cnn(C)c3)cn3nccc23)ccc1C]

A screw top vial was charged with 2-methyl-5-((6-(1-methyl-1H-pyrazol-4-yl)pyrazolo[1,5-a]pyrazin-4-yl)oxy)aniline (50 mg, 0.156 mmol) and THF (2 mL). To this was added acryloyl chloride (24.5 mg, 0.234 mmol), followed by TEA (23.7 mg, 0.234 mmol) with stirring at RT for 3 h. The reaction mixture was evaporated to dryness in vacuo and the residue purified by prep HPLC (Waters XSelect CSH Prep C18 5 μm OBD 19×100 mm; Gradient: 10-95% MeCN:H₂O w/0.1% NH₄OH modifier) to give (E)-N-(2-methyl-5-((6-(1-methyl-1H-pyrazol-4-yl)pyrazolo[1,5-a]pyrazin-4-yl)oxy)phenyl)but-2-enamide as a white solid (28 mg, 44% yield, 95% purity). LCMS m/z=389.4 (M+H)⁺; ¹H NMR (400 MHz, DMSO-d₆) δ 9.17-9.40 (m, 1H), 8.67-8.91 (m, 1H), 7.89-8.16 (m, 2H), 7.76-7.86 (m, 1H), 7.54-7.86 (m, 1H), 7.19-7.32 (m, 1H), 7.00-7.10 (m, 1H), 6.88-6.98 (m, 1H), 6.62-6.82 (m, 1H), 6.12-6.30 (1H, m), 3.76 (3, 3H), 2.14-2.31 (m, 3H), 1.71-1.91 (m, 3H).

### Example 132. N-(3-((6-(1-methyl-1H-pyrazol-4-yl)pyrazolo[1,5-a]pyrazin-4-yl)oxy)benzyl)acrylamide

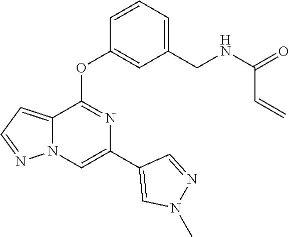 [SMILES: C=CC(=O)NCc1cccc(Oc2nc(-c3cnn(C)c3)cn3nccc23)c1]

N-(3-((6-(1-methyl-1H-pyrazol-4-yl)pyrazolo[1,5-a]pyrazin-4-yl)oxy)benzyl)acrylamide was obtained as a pale yellow solid, from 4-chloro-6-(1-methyl-1H-pyrazol-4-yl)pyrazolo[1,5-a]pyrazine (Intermediate A) and 3-(aminomethyl)phenol following a similar procedure to that described for the synthesis of Example 131. The compound was purified by prep HPLC (Waters SunFire Prep, C18 5 μm, OED 30×50 mm, eluting with 10-95% MeCN:H₂O (with 0.1% TFA modifier)). LCMS m/z=375.1 (M+H)⁺; ¹H NMR (400 MHz, DMSO-d₆) δ=8.93-8.87 (m, 1H), 8.76-8.64 (m, 1H), 8.15-8.10 (m, 1H), 7.92-7.87 (m, 1H), 7.86-7.80 (m, 1H), 7.50-7.43 (m, 1H), 7.34-7.19 (m, 3H), 7.05-6.97 (m, 1H), 6.36-6.22 (m, 1H), 6.19-6.05 (m, 1H), 5.67-5.55 (m, 1H), 4.49-4.39 (m, 2H), 3.83 (s, 3H)

### Example 133. N-(2-chloro-3-((6-(1-methyl-1H-pyrazol-4-yl)pyrazolo[1,5-a]pyrazin-4-yl)oxy)phenyl)-3-cyclopropylpropiolamide

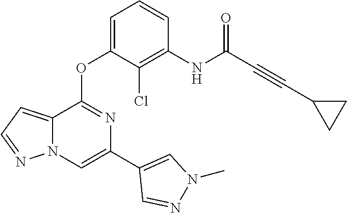 [SMILES: Cn1cc(-c2cn3nccc3c(Oc3cccc(NC(=O)C#CC4CC4)c3Cl)n2)cn1]

### 1. Synthesis of 2-chloro-3-((6-(1-methyl-1H-pyrazol-4-yl)pyrazolo[1,5-a]pyrazin-4-yl)oxy)aniline

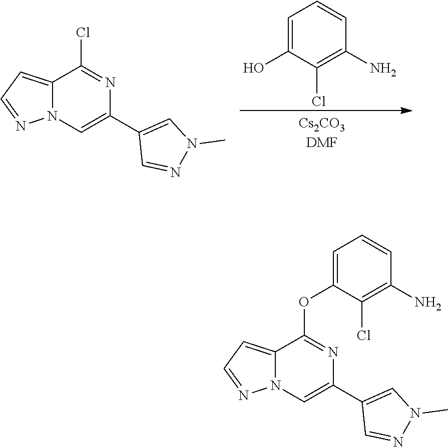 [SMILES: Cn1cc(-c2cn3nccc3c(Cl)n2)cn1 ; Nc1cccc(O)c1Cl ; Cn1cc(-c2cn3nccc3c(Oc3cccc(N)c3Cl)n2)cn1]

Cs₂CO₃ (335 mg, 1.03 mmol) was added to a mixture of 4-chloro-6-(1-methylpyrazol-4-yl)pyrazolo[1,5-a]pyrazine (Intermediate A, 240 mg, 1.03 mmol), 3-amino-2-chloro-phenol (155 mg, 1.08 mmol) and dry DMF (3 mL) the vial sealed and heated in a MW oven at 150° C. for 15 mins. The cooled mixture was diluted with water and extracted with EtOAc (2×). The combined organic extracts were washed with brine, dried over Na₂SO₄, filtered and evaporated under reduced pressure. The crude product was purified on a 10 g SPE column eluting with a heptane/EtOAc gradient to give 2-chloro-3-(6-(1-methylpyrazol-4-yl)pyrazolo[1,5-a]pyrazin-4-yl)oxy-aniline (170 mg, 44% yield) as a yellow solid. LCMS m/z=341.3, 353.3 (M+H)+

### 2. Synthesis of N-(2-chloro-3-((6-(1-methyl-1H-pyrazol-4-yl)pyrazolo[1,5-a]pyrazin-4-yl)oxy)phenyl)-3-cyclopropylpropiolamide

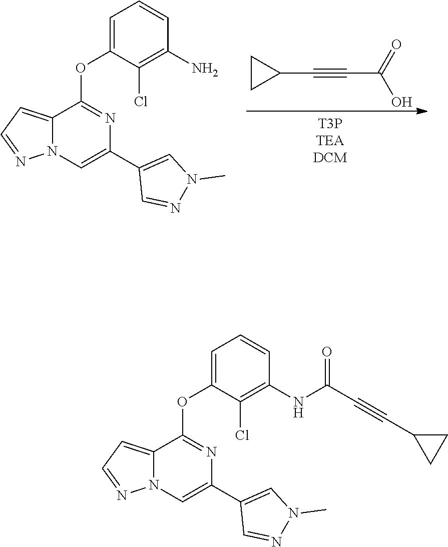 [SMILES: Cn1cc(-c2cn3nccc3c(Oc3cccc(N)c3Cl)n2)cn1 ; O=C(O)C#CC1CC1 ; Cn1cc(-c2cn3nccc3c(Oc3cccc(NC(=O)C#CC4CC4)c3Cl)n2)cn1]

TEA (36 mg, 0.352 mmol) followed by T3P (187 mg, 0.293 mmol) were added to a solution of 2-chloro-3-(6-(1-methylpyrazol-4-yl)pyrazolo[1,5-a]pyrazin-4-yl)oxy-aniline (40 mg, 0.117 mmol) and 3-cyclopropylprop-2-ynoic acid (26 mg, 0.235 mmol) in DCM (1 mL) and the reaction stirred at RT overnight. The mixture was diluted with water and extracted with EtOAc (2×). The combined organic extracts were washed with brine, dried over Na₂SO₄, filtered and the filtrate was evaporated under reduced pressure. The residual material was purified by prep HPLC (Waters SunFire Prep, C18 5 μm, OED 30×50 mm, eluting with 10-95% MeCN:H₂O [with 0.1% TFA modifier]): to give N-(2-chloro-3-((6-(1-methyl-1H-pyrazol-4-yl)pyrazolo[1,5-a]pyrazin-4-yl)oxy)phenyl)-3-cyclopropylpropiolamide (11 mg, 16% yield) as a white solid after lyophylisation. LCMS m/z=433.3 (M+H)+. ¹H NMR (400 MHz, DMSO-d₆) δ 10.48-10.20 (m, 1H), 9.02-8.82 (m, 1H), 8.25-8.07 (m, 1H), 7.91-7.72 (m, 2H), 7.58-7.36 (m, 3H), 7.18-7.03 (m, 1H), 3.81 (s, 3H), 1.73-1.44 (m, 1H), 1.12-0.72 (m, 4H).

### Example 134. N-(5-fluoro-2-methyl-3-((6-(1-methyl-1H-pyrazol-4-yl)pyrazolo[1,5-a]pyrazin-4-yl)oxy)phenyl)cyclobut-1-ene-1-carboxamide

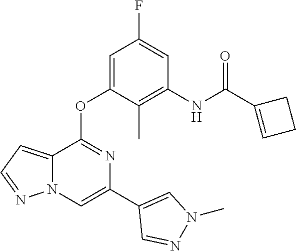 [SMILES: Cc1c(NC(=O)C2=CCC2)cc(F)cc1Oc1nc(-c2cnn(C)c2)cn2nccc12]

### 1. Synthesis of 5-fluoro-2-methyl-3-((6-(1-methyl-1H-pyrazol-4-yl)pyrazolo[1,5-a]pyrazin-4-yl)oxy)aniline

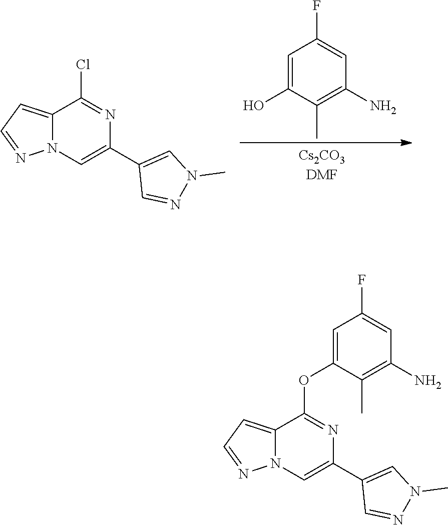

5-Fluoro-2-methyl-3-((6-(1-methyl-1H-pyrazol-4-yl)pyrazolo[1,5-a]pyrazin-4-yl)oxy)aniline was obtained as a yellow solid from 4-chloro-6-(1-methylpyrazol-4-yl)pyrazolo[1,5-a]pyrazine (Intermediate A) and 3-amino-5-fluoro-2-methylphenol, following the procedure described in step 1, Example 133. LCMS m/z=339.3 (M+H)+

### 2. Synthesis of N-(5-fluoro-2-methyl-3-((6-(1-methyl-1H-pyrazol-4-yl)pyrazolo[1,5-a]pyrazin-4-yl)oxy)phenyl)cyclobut-1-ene-1-carboxamide

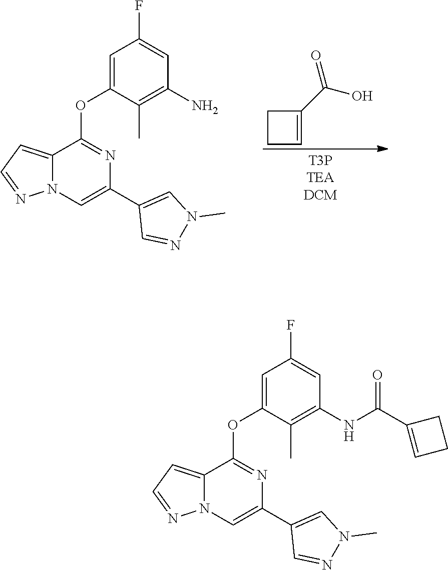 [SMILES: Cc1c(N)cc(F)cc1Oc1nc(-c2cnn(C)c2)cn2nccc12 ; O=C(O)C1=CCC1 ; Cc1c(NC(=O)C2=CCC2)cc(F)cc1Oc1nc(-c2cnn(C)c2)cn2nccc12]

N-(5-Fluoro-2-methyl-3-((6-(1-methyl-1H-pyrazol-4-yl)pyrazolo[1,5-a]pyrazin-4-yl)oxy)phenyl)cyclobut-1-ene-1-carboxamide was obtained as a white solid, 4 mg, 6% yield from 5-fluoro-2-methyl-3-((6-(1-methyl-1H-pyrazol-4-yl)pyrazolo[1,5-a]pyrazin-4-yl)oxy)aniline and cyclobutene-1-carboxylic acid, following the procedure described in step 2, Example 133. LCMS m/z=419.4 (M+H)+. ¹H NMR (400 MHz, DMSO-d₆) δ 9.66-9.44 (m, 1H), 8.94-8.71 (m, 1H), 8.18-7.98 (m, 1H), 7.90-7.80 (m, 1H), 7.76-7.68 (m, 1H), 7.24-7.09 (m, 2H), 7.07-6.97 (m, 1H), 6.82-6.61 (m, 1H), 3.86-3.63 (m, 3H), 2.74-2.57 (m, 2H), 2.40-2.29 (m, 2H), 1.97-1.80 (m, 3H).

### Example 135. (E)-4-(cyclobutyl(methyl)amino)-N-(2-methyl-3-((6-(1-methyl-1H-pyrazol-4-yl)pyrazolo[1,5-a]pyrazin-4-yl)oxy)phenyl)but-2-enamide

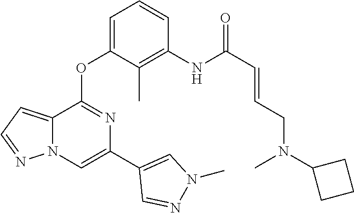 [SMILES: Cc1c(NC(=O)/C=C/CN(C)C2CCC2)cccc1Oc1nc(-c2cnn(C)c2)cn2nccc12]

### 1. Synthesis of 2-methyl-3-((6-(1-methyl-1H-pyrazol-4-yl)pyrazolo[1,5-a]pyrazin-4-yl)oxy)aniline

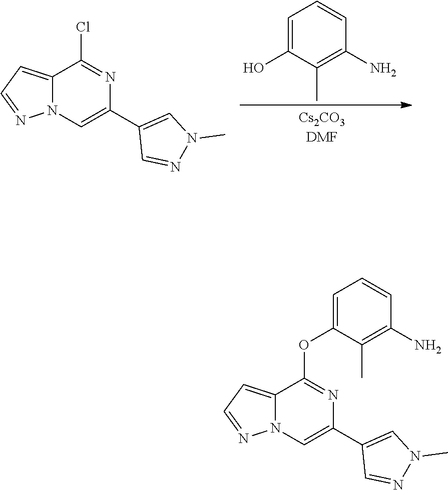

2-Methyl-3-((6-(1-methyl-1H-pyrazol-4-yl)pyrazolo[1,5-a]pyrazin-4-yl)oxy)aniline was obtained as a pale-yellow solid, from 4-chloro-6-(1-methylpyrazol-4-yl)pyrazolo[1,5-a]pyrazine (Intermediate A) and 3-amino-2-methylphenol, following the procedure described in step 1, Example 133. ¹H NMR (400 MHz, DMSO-d₆) δ 8.88-8.75 (m, 1H), 8.19-8.03 (m, 1H), 7.88-7.70 (m, 2H), 7.09-6.87 (m, 2H), 6.68-6.55 (m, 1H), 6.51-6.37 (m, 1H), 5.16-4.93 (m, 2H), 3.90-3.74 (m, 3H).

### 2. Synthesis of (E) and (Z)-4-chloro-N-(2-methyl-3-((6-(1-methyl-1H-pyrazol-4-yl)pyrazolo[1,5-a]pyrazin-4-yl)oxy)phenyl)but-2-enamide

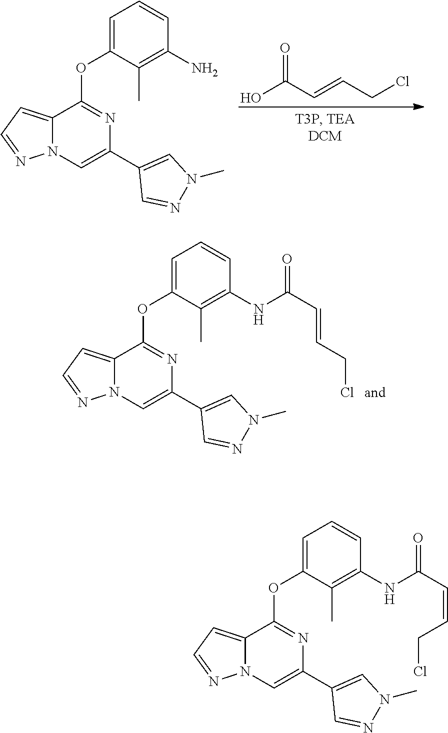

TEA (303 mg, 3.0 mmol) followed by T3P (1.59 g, 2.50 mmol, 50% purity) were added to 2-chloro-3-((6-(1-methyl-1H-pyrazol-4-yl)pyrazolo[1,5-a]pyrazin-4-yl)oxy)aniline (320 mg, 1.0 mmol), (E)-4-chlorobut-2-enoic acid (268 mg, 2.0 mmol) and DCM (7 mL) and the reaction stirred for 2 h. The mixture was diluted with water and extracted with EtOAc (2×). The combined organic extracts were washed with brine, dried over Na₂SO₄, filtered and the filtrate evaporated under reduced pressure. The residual material was purified on a 10 g Si-SPE column eluting with EtOAc to give (E)-4-chloro-N-[2-methyl-3-[6-(1-methylpyrazol-4-yl)pyrazolo[1,5-a]pyrazin-4-yl]oxy-phenyl]but-2-enamide (340 mg, 72% yield, 90% purity) and (Z)-4-chloro-N-[2-methyl-3-[6-(1-methylpyrazol-4-yl)pyrazolo[1,5-a]pyrazin-4-yl]oxy-phenyl]but-2-enamide as a pale yellow solid. LCMS m/z 423.4 (M+H)+

### 3. Synthesis of (E)-4-(cyclobutyl(methyl)amino)-N-(2-methyl-3-((6-(1-methyl-1H-pyrazol-4-yl)pyrazolo[1,5-a]pyrazin-4-yl)oxy)phenyl)but-2-enamide

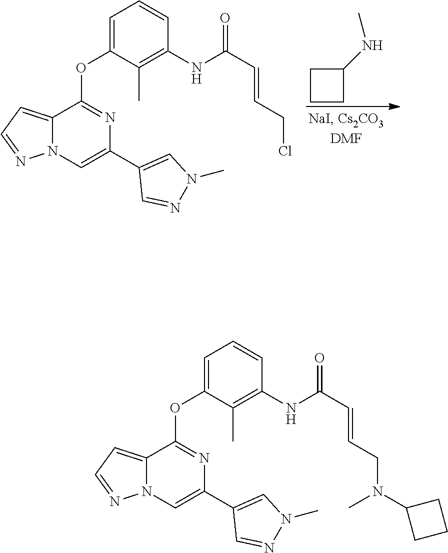

A screwtop vial charged with (E)-4-chloro-N-[2-methyl-3-[6-(1-methylpyrazol-4-yl)pyrazolo[1,5-a]pyrazin-4-yl]oxy-phenyl]but-2-enamide (60 mg, 142 μmol), sodium iodide (21 mg, 142 μmol) and DMF (1 mL) and stirred at RT. N-Methylcyclobutanamine hydrochloride (52 mg, 426 μmol) followed by Cs₂CO₃ (185 mg, 568 μmol) were added that the mixture stirred at RT overnight. The mixture was diluted with water and the product extracted with EtOAc (2×). The combined organic extracts were dried over Na₂SO₄, filtered and the filtrate concentrated in vacuo. The residual material was purified by prep HPLC (Waters SunFire Prep, C18 5 μm, OED 30×50 mm, eluting with 10-95% MeCN:H₂O (with 0.1% TFA modifier) to give, (E)-4-(cyclobutyl(methyl)amino)-N-(2-methyl-3-((6-(1-methyl-1H-pyrazol-4-yl)pyrazolo[1,5-a]pyrazin-4-yl)oxy)phenyl)but-2-enamide (21.1 mg, 24% yield, 95% purity) as a white solid. LCMS m/z=472.5 (M+H)+. 1H NMR (400 MHz, DMSO-d₆) δ=9.97-9.88 (m, 1H), 9.87-9.75 (m, 1H), 8.92-8.84 (m, 1H), 8.19-8.10 (m, 1H), 7.94-7.88 (m, 1H), 7.82-7.73 (m, 1H), 7.49-7.41 (m, 1H), 7.38-7.30 (m, 1H), 7.29-7.18 (m, 1H), 7.12-7.04 (m, 1H), 6.83-6.70 (m, 1H), 6.65-6.50 (m, 1H), 4.03-3.91 (m, 1H), 3.81 (s, 3H), 3.77-3.66 (m, 1H), 2.71-2.62 (m, 3H), 2.26-2.11 (m, 4H), 2.06-1.98 (m, 3H), 1.84-1.64 (m, 2H)

### Example 136 (E)-4,4,4-trifluoro-N-(2-fluoro-3-((6-(1-(tetrahydrofuran-3-yl)-1H-pyrazol-4-yl)pyrazolo[1,5-a]pyrazin-4-yl)oxy)phenyl)but-2-enamide

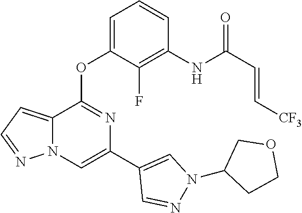 [SMILES: O=C(/C=C/C(F)(F)F)Nc1cccc(Oc2nc(-c3cnn(C4CCOC4)c3)cn3nccc23)c1F]

### 1. Synthesis of 3-((6-chloropyrazolo[1,5-a]pyrazin-4-yl)oxy)-2-fluoroaniline

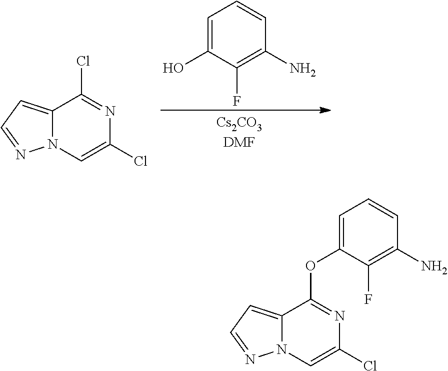 [SMILES: Clc1cn2nccc2c(Cl)n1 ; Nc1cccc(O)c1F ; Nc1cccc(Oc2nc(Cl)cn3nccc23)c1F]

Cs₂CO₃ (347 mg, 1.1 mmol) was added to a solution of 4,6-dichloropyrazolo[1,5-a]pyrazine (200 mg, 1.06 mmol) and 3-amino-2-fluoro-phenol (171 mg, 1.28 mmol) in dry DMF (2.5 mL) the vial sealed and heated in a MW oven at 150° C. for 15 mins. The cooled mixture was diluted with water, extracted with EtOAc (2×), the combined organic extracts washed with brine, dried over Na₂SO₄, filtered and evaporated under reduced pressure. The crude product was purified on a 10 g SPE cartridge using a heptane/EtOAc gradient to give 3-((6-chloropyrazolo[1,5-a]pyrazin-4-yl)oxy)-2-fluoroaniline (290 mg, 93% yield) as a pale-yellow solid. LCMS m/z=279.1, 281.2 (M+H)+

### 2. Synthesis of 2-fluoro-3-((6-(1-(tetrahydrofuran-3-yl)-1H-pyrazol-4-yl)pyrazolo[1,5-a]pyrazin-4-yl)oxy)aniline

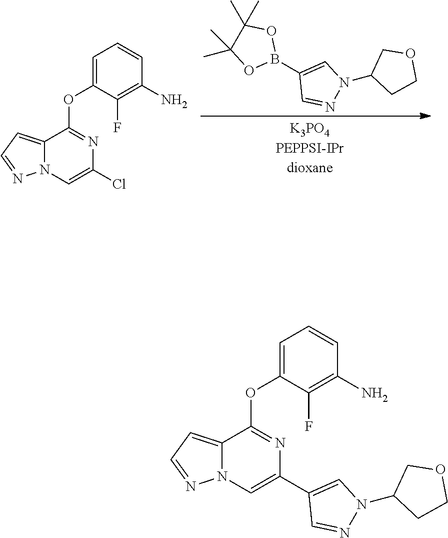 [SMILES: Nc1cccc(Oc2nc(Cl)cn3nccc23)c1F ; CC1(C)OB(c2cnn(C3CCOC3)c2)OC1(C)C ; Nc1cccc(Oc2nc(-c3cnn(C4CCOC4)c3)cn3nccc23)c1F]

Dioxane (2 mL) and water (1 mL) were added to a mixture of 3-(6-chloropyrazolo[1,5-a]pyrazin-4-yl)oxy-2-fluoro-aniline (142 mg, 0.510 mmol), 1-tetrahydrofuran-3-yl-4-(4,4,5,5-tetramethyl-1,3,2-dioxaborolan-2-yl)pyrazole (283 mg, 1.07 mmol), PEPPSI™-IPr catalyst (70 mg, 0.102 mmol) and K₃PO₄(325 mg, 1.53 mmol), the vial was sealed and heated in a MW oven at 150° C. for 15 mins. The cooled mixture was diluted with water and extracted with EtOAc (2×), the combined organic extracts were washed with brine, dried over Na₂SO₄, filtered and evaporated under reduced pressure. The crude product was purified on a 10 g SPE cartridge eluting with heptane/EtOAc to give 2-fluoro-3-((6-(1-(tetrahydrofuran-3-yl)-1H-pyrazol-4-yl)pyrazolo[1,5-a]pyrazin-4-yl)oxy)aniline (120 mg, 56% yield) as a pale-yellow solid. LCMS m/z=381.4 (M+H)+

### 3. Synthesis of (E)-4,4,4-trifluoro-N-(2-fluoro-3-((6-(1-(tetrahydrofuran-3-yl)-1H-pyrazol-4-yl)pyrazolo[1,5-a]pyrazin-4-yl)oxy)phenyl)but-2-enamide

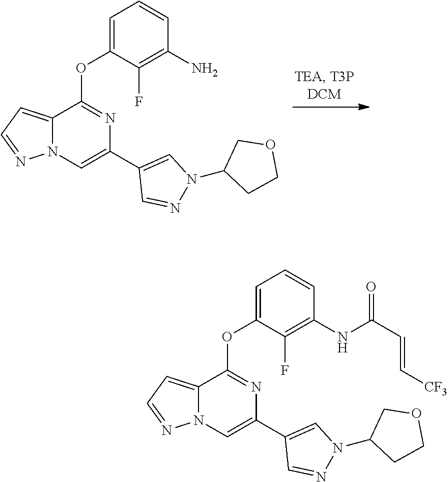 [SMILES: Nc1cccc(Oc2nc(-c3cnn(C4CCOC4)c3)cn3nccc23)c1F ; O=C(/C=C/C(F)(F)F)Nc1cccc(Oc2nc(-c3cnn(C4CCOC4)c3)cn3nccc23)c1F]

TEA (32 mg, 0.316 mmol) followed by T3P (167 mg, 0.263 mmol) were added to a mixture of 2-fluoro-3-((6-(1-(tetrahydrofuran-3-yl)-1H-pyrazol-4-yl)pyrazolo[1,5-a]pyrazin-4-yl)oxy)aniline (40 mg, 0.105 mmol), (E)-4,4,4-trifluorobut-2-enoic acid (29 mg, 0.21 mmol) in DCM (1 mL) and the reaction stirred at RT for 3 h. The mixture was diluted with water and extracted with EtOAc (2×). The combined organic extracts were washed with brine, dried over Na₂SO₄, filtered and the filtrate evaporated under reduced pressure. The residual material was purified by prep HPLC (Waters SunFire Prep, C18 5 μm, OED 30×50 mm, eluting with 10-95% MeCN:H₂O [with 0.1% TFA modifier]) to give after lyophylisation (E)-4,4,4-trifluoro-N-(2-fluoro-3-((6-(1-(tetrahydrofuran-3-yl)-1H-pyrazol-4-yl)pyrazolo[1,5-a]pyrazin-4-yl)oxy)phenyl)but-2-enamide (25 mg, 37% yield) as a white solid. LCMS m/z=503.4 (M+H)⁺. 1H NMR (400 MHz, DMSO-d₆) δ:10.56 (s, 1H), 8.97 (d, J=0.75 Hz, 1H), 8.18 (d, J=2.51 Hz, 1H), 8.03 (s, 1H), 7.91-8.01 (m, 1H), 7.79 (s, 1H), 7.31-7.40 (m, 2H), 7.18 (dd, J=1.88, 15.44 Hz, 1H), 7.12 (dd, J=0.88, 2.38 Hz, 1H), 6.93-7.05 (m, 1H), 4.95-5.06 (m, 1H), 3.89-3.99 (m, 2H), 3.76-3.86 (m, 2H), 2.29-2.41 (m, 1H), 2.15-2.25 (m, 1H)

### Example 137: N-(1-(2-methyl-3-((6-(1-methyl-1H-pyrazol-4-yl)pyrazolo[1,5-a]pyrazin-4-yl)oxy)phenyl)ethyl)acrylamide

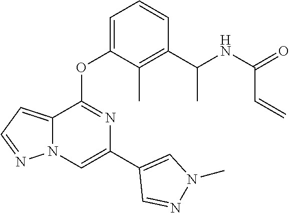 [SMILES: C=CC(=O)NC(C)c1cccc(Oc2nc(-c3cnn(C)c3)cn3nccc23)c1C]

### 1. Synthesis of 2-methyl-3-((6-(1-methyl-1H-pyrazol-4-yl)pyrazolo[1,5-a]pyrazin-4-yl)oxy)benzonitrile

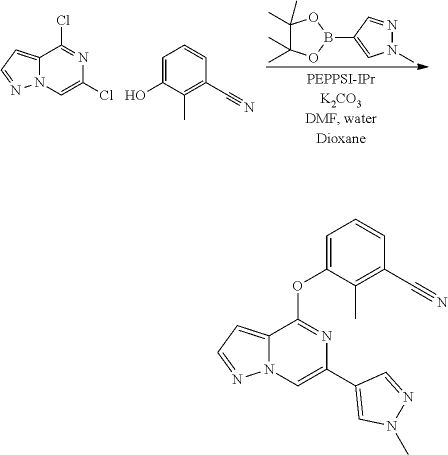

A mixture of 4,6-dichloropyrazolo[1,5-a]pyrazine (740 mg, 3.94 mmol), 3-hydroxy-2-methyl-benzonitrile (525 mg, 3.94 mmol) and K₂CO₃ (1.65 g, 12.0 mmol) in DMF (5 mL) was heated to 100° C. for 20 mins in a Biotage MW reactor. 1-Methyl-4-(4,4,5,5-tetramethyl-1,3,2-dioxaborolan-2-yl)pyrazole (1.5 g, 7.21 mmol), PEPPSI™-IPr catalyst (55 mg, 0.08 mmol), dioxane (5 mL) and water (2.50 mL) were added, the reaction vial was sealed and heated to 100° C. for 1 h in a Biotage MW reactor. The cooled reaction mixture was diluted with water (10 mL) and extracted with EtOAc (3×10 mL). The combined organic layers were concentrated to dryness and purified by column chromatography (heptane/EtOAc) to give 2-methyl-3-((6-(1-methyl-1H-pyrazol-4-yl)pyrazolo[1,5-a]pyrazin-4-yl)oxy)benzonitrile. LCMS m/z=331.1 (M+H)+

### 2. Synthesis of 1-(2-methyl-3-((6-(1-methyl-1H-pyrazol-4-yl)pyrazolo[1,5-a]pyrazin-4-yl)oxy)phenyl)ethan-1-imine hydrochloride

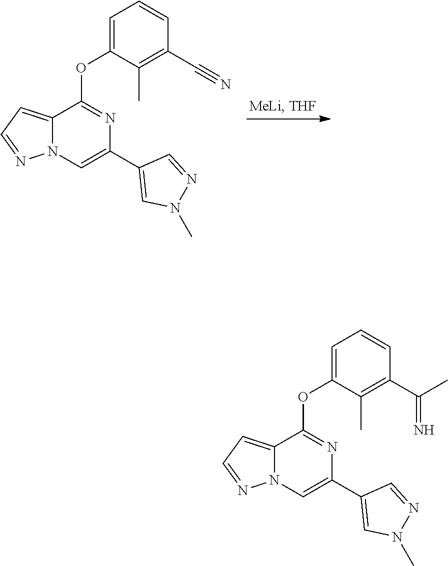 [SMILES: Cc1c(C#N)cccc1Oc1nc(-c2cnn(C)c2)cn2nccc12 ; CC(=N)c1cccc(Oc2nc(-c3cnn(C)c3)cn3nccc23)c1C]

A mixture of 2-methyl-3-((6-(1-methyl-1H-pyrazol-4-yl)pyrazolo[1,5-a]pyrazin-4-yl)oxy)benzonitrile (110 mg, 0.333 mmol) in THF (1 mL) was cooled to −78° C. Methyllithium (1.6 M, 333 uL) was added drop-wise and the reaction stirred at −78° C. for 2 h. The reaction was quenched with MeOH, the mixture allowed to warm to RT and stirred for 2 h. The mixture was diluted with THF, then filtered through a hydrophobic frit. The filtrate was evaporated under reduced pressure, the residual material was dissolved in MTBE and treated with HCl/Et₂O. The mixture was evaporated under reduced pressure to give 1-(2-methyl-3-((6-(1-methyl-1H-pyrazol-4-yl)pyrazolo[1,5-a]pyrazin-4-yl)oxy)phenyl)ethan-1-imine hydrochloride. LCMS m/z=347.1 (M+H)+

### 3. Synthesis of 1-(2-methyl-3-((6-(1-methyl-1H-pyrazol-4-yl)pyrazolo[1,5-a]pyrazin-4-yl)oxy)phenyl)ethan-1-amine

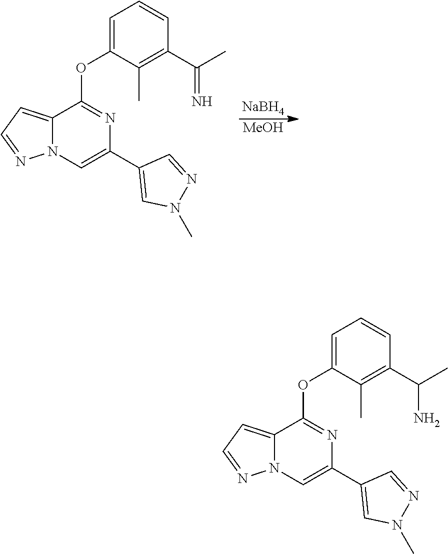 [SMILES: "MeOH" ; CC(=N)c1cccc(Oc2nc(-c3cnn(C)c3)cn3nccc23)c1C ; "NaBH4" ; Cc1c(Oc2nc(-c3cnn(C)c3)cn3nccc23)cccc1C(C)N]

NaBH₄ (11 mg, 0.287 mmol) was added to a solution of 1-(2-methyl-3-((6-(1-methyl-1H-pyrazol-4-yl)pyrazolo[1,5-a]pyrazin-4-yl)oxy)phenyl)ethan-1-imine hydrochloride (110 mg, 0.287 mmol) in MeOH (3 mL) in an ice bath and the reaction stirred at RT overnight. Water was carefully added, followed by DCM and the mixture was stirred vigorously for 10 mins and then poured on to a hydrophobic frit. The filtrate was concentrated in vacuo and the residue was purified on a 5 g SCX column eluting with 2M NH₃-MeOH to give 1-(2-methyl-3-((6-(1-methyl-1H-pyrazol-4-yl)pyrazolo[1,5-a]pyrazin-4-yl)oxy)phenyl)ethan-1-amine (50 mg, 45% yield) as a yellow solid. LCMS m/z=349.1 (M+H)+

### 4. Synthesis of N-(1-(2-methyl-3-((6-(1-methyl-1H-pyrazol-4-yl)pyrazolo[1,5-a]pyrazin-4-yl)oxy)phenyl)ethyl)acrylamide

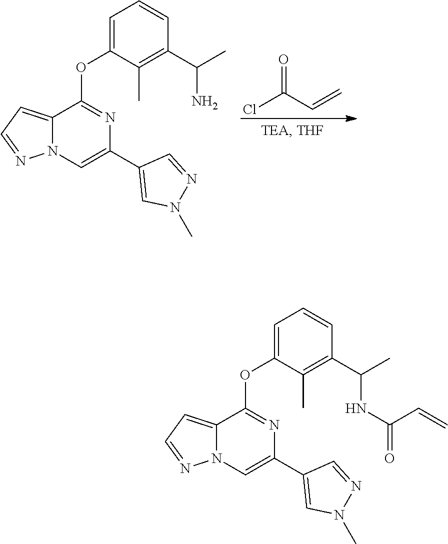

Acryloyl chloride (19.5 mg, 0.215 mmol) was added to a solution of 1-(2-methyl-3-((6-(1-methyl-1H-pyrazol-4-yl)pyrazolo[1,5-a]pyrazin-4-yl)oxy)phenyl)ethan-1-amine (50 mg, 0.144 mmol) in THF (2 mL). TEA (21.8 mg, 0.215 mmol) was added and the reaction stirred at RT overnight. The mixture was concentrated in vacuo and the residue was purified by prep HPLC (Waters SunFire Prep, C18 5 μm, OED 30×50 mm, eluting with 10-95% MeCN:H₂O (with 0.I % TFA modifier) to give N-(1-(2-methyl-3-((6-(1-methyl-1H-pyrazol-4-yl)pyrazolo[1,5-a]pyrazin-4-yl)oxy)phenyl)ethyl)acrylamide (10.8 mg, 14% yield) as a white solid after lyophylisation. 1HNMR (400 MHz, DMSO-d₆) δ: 8.86-8.73 (m, 1H), 8.68-8.53 (m, 1H), 8.13-7.99 (m, 1H), 7.83-7.66 (m, 2H), 7.33-7.18 (m, 2H), 7.16-7.06 (m, 1H), 7.02-6.90 (m, 1H), 6.36-6.16 (m, 1H), 6.11-5.94 (m, 1H), 5.62-5.46 (m, 1H), 5.21-5.03 (m, 1H), 3.84-3.72 (m, 14H), 2.17-1.98 (m, 3H), 1.44-1.25 (m, 3H).

### Example 138: N-(5-((6-(1-methyl-1H-pyrazol-4-yl)pyrazolo[1,5-a]pyrazin-4-yl)oxy)pyridin-3-yl)but-2-ynamide

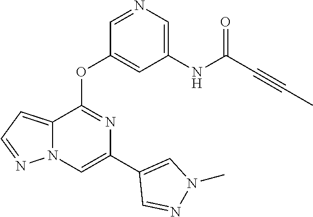 [SMILES: CC#CC(=O)Nc1cncc(Oc2nc(-c3cnn(C)c3)cn3nccc23)c1]

### 1. Synthesis of tert-butyl (5-((6-(1-methyl-1H-pyrazol-4-yl)pyrazolo[1,5-a]pyrazin-4-yl)oxy)pyridin-3-yl)carbamate

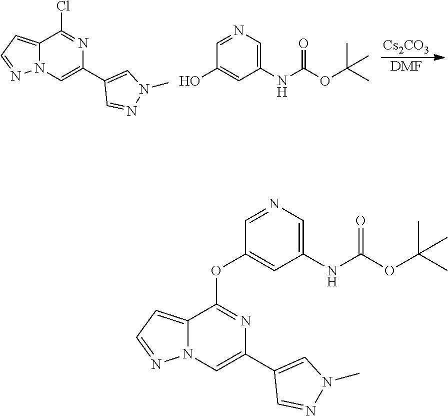

To a microwave vial was added 4-chloro-6-(1-methyl-1H-pyrazol-4-yl)pyrazolo[1,5-a]pyrazine (100 mg, 428 μmol), tert-butyl (5-hydroxypyridin-3-yl)carbamate (90 mg, 428 μmol) and dry DMF (1.42 mL). Cs₂CO₃ (139 mg, 428 μmol) was added, and the vessel was heated in a microwave at 110° C. for 10 mins. After cooling to ambient temperature, the mixture was diluted with water and extracted twice with EtOAc. The crude product was minimally soluble in EtOAc and crashed out of solution upon workup. The heterogeneous solution was filtered, and the precipitate was dried under vacuum. The crude precipitate was isolated to give tert-butyl (5-((6-(1-methyl-1H-pyrazol-4-yl)pyrazolo[1,5-a]pyrazin-4-yl)oxy)pyridin-3-yl)carbamate as a white solid (94 mg, 54% yield) and was carried forward without further purification. ESI-MS (M+H)⁺: 408.4. ¹H NMR (500 MHz, DMSO-d₆) δ: 9.86 (s, 1H), 8.94 (s, 1H), 8.53 (d, J=1.8 Hz, 1H), 8.30 (d, J=2.4 Hz, 1H), 8.15 (d, J=2.4 Hz, 2H), 7.96 (s, 1H), 7.87 (s, 1H), 7.07 (d, J=2.4 Hz, 1H), 3.82 (s, 3H), 1.49 (s, 9H).

### 2. Synthesis of 5-((6-(1-methyl-1H-pyrazol-4-yl)pyrazolo[1,5-a]pyrazin-4-yl)oxy)pyridin-3-amine dihydrochloride

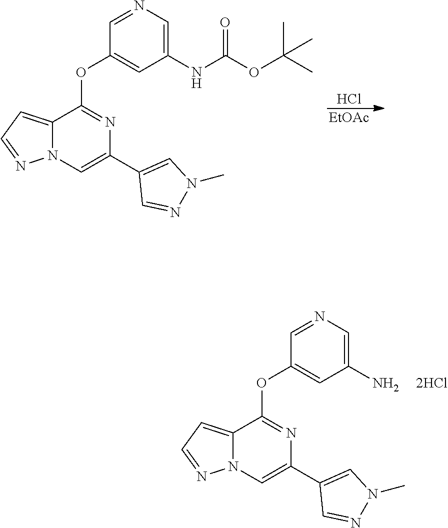 [SMILES: "EtOAc" ; Cn1cc(-c2cn3nccc3c(Oc3cncc(NC(=O)OC(C)(C)C)c3)n2)cn1 ; Cl ; Cn1cc(-c2cn3nccc3c(Oc3cncc(N)c3)n2)cn1 ; "2HCl"]

To a suspension of tert-butyl (5-((6-(1-methyl-1H-pyrazol-4-yl)pyrazolo[1,5-a]pyrazin-4-yl)oxy)pyridin-3-yl)carbamate (94 mg, 231 μmol) in EtOAc (0.5 mL) was added an HCl solution (1M in EtOAc, 2.31 mL). The reaction mixture was stirred at ambient temperature for 3 d. The reaction mixture was concentrated to give 5-((6-(1-methyl-1H-pyrazol-4-yl)pyrazolo[1,5-a]pyrazin-4-yl)oxy)pyridin-3-amine dihydrochloride (crude, assuming quantitative yield), which was carried forward without further purification. ESI-MS (M+H)+: 308.3.

### 3. Synthesis of N-(5-((6-(1-methyl-1H-pyrazol-4-yl)pyrazolo[1,5-a]pyrazin-4-yl)oxy)pyridin-3-yl)but-2-ynamide

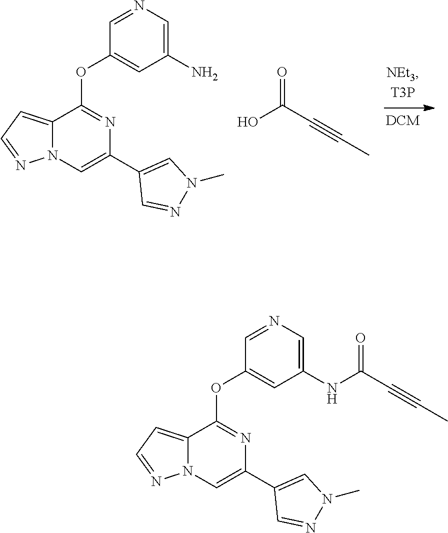

To a solution of 5-((6-(1-methyl-1H-pyrazol-4-yl)pyrazolo[1,5-a]pyrazin-4-yl)oxy)pyridin-3-amine dihydrochloride (crude, 231 μmol) in DCM (2 mL) was added but-2-ynoic acid (40 mg, 473 μmol), NEt₃ (144 mg, 1.42 mmol), and T3P (377 mg, 592 μmol, 50% purity). The reaction mixture was stirred at ambient temperature for 18 h before water was added. The phases were separated, and the aqueous phase was washed with EtOAc twice. The combined organic extracts were washed with brine, dried (Na₂SO₄), filtered, and concentrated. The crude material was purified via reverse phase purification (Column: Waters XSelect CSH Prep C18 5 um OBD 19×100 mm; Condition: 5-50% MeCN in 0.1% v/v NH₄CO₃/water; Flow rate: 30 mL/min) to afford N-(5-((6-(1-methyl-1H-pyrazol-4-yl)pyrazolo[1,5-a]pyrazin-4-yl)oxy)pyridin-3-yl)but-2-ynamide as an amber solid (15.1 mg, 17% yield). ESI-MS (M+H)+: 374.4. ¹H NMR (500 MHz, DMSO-d₆) δ:11.08 (br s, 1H), 8.94 (d, J=1.2 Hz, 1H), 8.67 (d, J=1.8 Hz, 1H), 8.42 (d, J=2.4 Hz, 1H), 8.24 (t, J=2.1 Hz, 1H), 8.15 (d, J=2.4 Hz, 1H), 7.94 (s, 1H), 7.84 (s, 1H), 7.11-7.05 (m, 1H), 3.82 (s, 3H), 2.08 (s, 3H).

### Examples 139, 140 & 141: racemic N-methyl-N-((1S,3R)-2,2,3-trimethyl-3-((6-(1-methyl-1H-pyrazol-4-yl)pyrazolo[1,5-a]pyrazin-4-yl)oxy)cyclobutyl)acrylamide; N-methyl-N-((1S,3R)-2,2,3-trimethyl-3-((6-(1-methyl-1H-pyrazol-4-yl)pyrazolo[1,5-a]pyrazin-4-yl)oxy)cyclobutyl)acrylamide and N-methyl-N-((1R,3S)-2,2,3-trimethyl-3-((6-(1-methyl-1H-pyrazol-4-yl)pyrazolo[1,5-a]pyrazin-4-yl)oxy)cyclobutyl)acrylamide

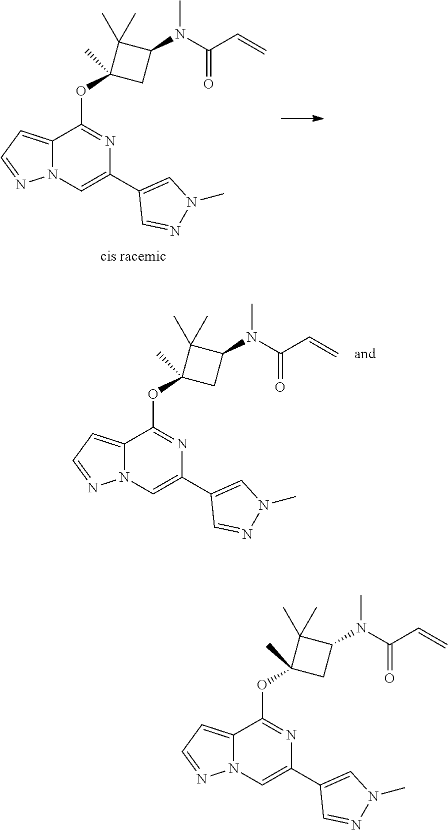 [SMILES: C=CC(=O)N(C)[C@H]1C[C@@](C)(Oc2nc(-c3cnn(C)c3)cn3nccc23)C1(C)C ; C=CC(=O)N(C)[C@H]1C[C@@](C)(Oc2nc(-c3cnn(C)c3)cn3nccc23)C1(C)C ; "and" ; C=CC(=O)N(C)[C@@H]1C[C@](C)(Oc2nc(-c3cnn(C)c3)cn3nccc23)C1(C)C]

### 1. Synthesis of racemic tert-butyl ((1S,3R)-3-hydroxy-2,2,3-trimethylcyclobutyl)carbamate

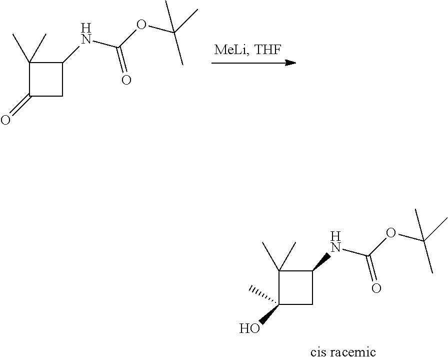 [SMILES: CC(C)(C)OC(=O)NC1CC(=O)C1(C)C ; CC(C)(C)OC(=O)N[C@H]1C[C@@](C)(O)C1(C)C]

An oven dried flask was charged with THF (75 mL), followed by a methyllithium solution (1.6 M in ether, 59 mL, 94 mmol) and the mixture was cooled to −78° C. A second oven-dried flask was charged with THF (75 mL), followed by tert-butyl (2,2-dimethyl-3-oxocyclobutyl)carbamate (5 g, 23 mmol) and cooled to −78° C. and this solution added to the cooled solution of methyllithium via cannula and the reaction mixture was stirred at −78° C. for 1 h. Saturated aq. NH₄Cl solution (150 mL) was added, followed by EtOAc (100 mL) and the layers were separated. The organic phase was washed with brine (100 mL), dried (Na₂SO₄), filtered, and concentrated. The crude material was purified by silica-gel column chromatography (from 0% to 50% EtOAc in heptanes) to give racemic tert-butyl ((1S,3R)-3-hydroxy-2,2,3-trimethylcyclobutyl)carbamate as a colorless oil (4.04 g, 80% yield). ¹H NMR (500 MHz, CHLOROFORM-d) δ: 4.73-4.38 (m, 1H), 3.59-3.27 (m, 1H), 2.32 (br dd, J=11.3 Hz, 8.2 Hz, 1H), 1.88-1.63 (m, 2H), 1.50-1.38 (m, 9H), 1.27 (s, 3H), 1.05 (s, 3H), 1.00 (s, 3H).

### 2. Synthesis of racemic tert-butyl methyl((1S,3R)-2,2,3-trimethyl-3-((6-(1-methyl-1H-pyrazol-4-yl)pyrazolo[1,5-a]pyrazin-4-yl)oxy)cyclobutyl)carbamate

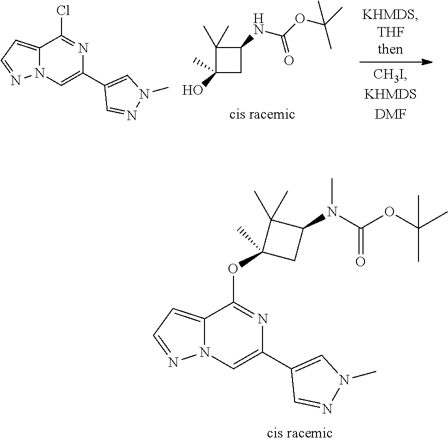

To a 0° C. cooled solution of racemic tert-butyl ((1S,3R)-3-hydroxy-2,2,3-trimethylcyclobutyl)carbamate (2.0 g, 8.72 mmol) in dry THF (28 mL) under a N₂ atmosphere was added a KHMDS solution (0.5 M in toluene, 34.9 mL, 17.4 mmol) and the reaction mixture was stirred at 0° C. for 15 mins. A solution of 4-chloro-6-(1-methyl-1H-pyrazol-4-yl)pyrazolo[1,5-a]pyrazine (1.85 g, 7.93 mmol) in DMSO (28 mL) was added and the reaction was stirred as it warmed to ambient temperature over 30 mins. An additional portion of the KHMDS solution (0.5 M in toluene, 15.9 mL, 7.93 mmol) was added, followed by iodomethane (987 μL, 15.9 mmol) and the reaction mixture stirred for 30 mins. Water (10 mL) was added, followed by 1N HCl solution until pH=7. EtOAc (100 mL) and the phases were separated. The organic phase was washed sequentially with water (100 mL) and brine (100 mL), dried (Na₂SO₄), filtered, and concentrated. The crude material was purified by silica-gel column chromatography (from 0% to 50% EtOAc in heptanes) to give racemic tert-butyl methyl((1S,3R)-2,2,3-trimethyl-3-((6-(1-methyl-1H-pyrazol-4-yl)pyrazolo[1,5-a]pyrazin-4-yl)oxy)cyclobutyl)carbamate (2.54 g, 73% yield). ESI-MS (M+H)⁺: 441.6. ¹H NMR (500 MHz, CDCl₃) δ: 8.24-8.17 (m, 1H), 7.90-7.84 (m, 2H), 7.76-7.72 (m, 1H), 6.73-6.66 (m, 1H), 3.98 (s, 3H), 3.95-3.87 (m, 1H), 2.88 (s, 3H), 2.85-2.78 (m, 1H), 2.73-2.65 (m, 1H), 1.78 (s, 3H), 1.49 (s, 9H), 1.36 (s, 3H), 1.11 (s, 3H).

### 3. Example 139: Synthesis of racemic (1S,3R)—N,2,2,3-tetramethyl-3-((6-(1-methyl-1H-pyrazol-4-yl)pyrazolo[1,5-a]pyrazin-4-yl)oxy)cyclobutan-1-amine

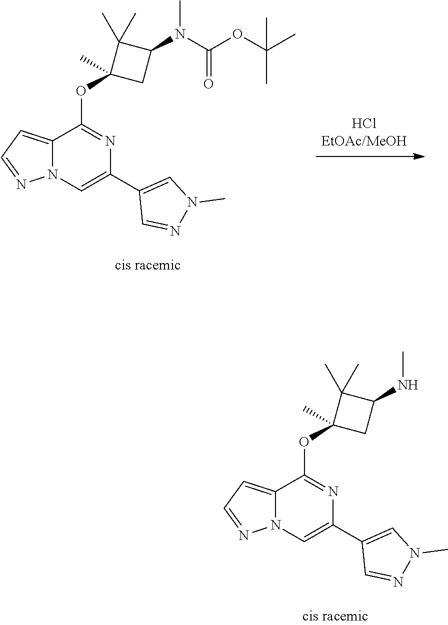 [SMILES: CN(C(=O)OC(C)(C)C)[C@H]1C[C@@](C)(Oc2nc(-c3cnn(C)c3)cn3nccc23)C1(C)C ; CN[C@H]1C[C@@](C)(Oc2nc(-c3cnn(C)c3)cn3nccc23)C1(C)C]

To a solution of racemic tert-butyl methyl((1S,3R)-2,2,3-trimethyl-3-((6-(1-methyl-1H-pyrazol-4-yl)pyrazolo[1,5-a]pyrazin-4-yl)oxy)cyclobutyl)carbamate (2.54 g, 5.76 mmol) in MeOH (5 mL) was added an HCl solution (1 M in EtOAc, 58 mL) and the reaction stirred at ambient temperature for 36 h. The reaction mixture was concentrated and reconstituted with water and EtOAc. The layers were separated and the acidic aqueous phase was extracted with EtOAc. The combined organic extracts were concentrated to recover unreacted starting material. To the acidic aqueous phase was added saturated aqueous sodium bicarbonate solution until pH=8-9. The entire basic aqueous phase was lyophilized and reconstituted in MeOH. The MeOH phase was filtered and concentrated to give racemic (1S,3R)—N,2,2,3-tetramethyl-3-((6-(1-methyl-1H-pyrazol-4-yl)pyrazolo[1,5-a]pyrazin-4-yl)oxy)cyclobutan-1-amine as a light yellow solid (1.75 g, 89% yield, crude). ESI-MS (M+H)⁺: 341.4.

### 4. Synthesis and chiral separation of racemic N-methyl-N-((1S,3R)-2,2,3-trimethyl-3-((6-(1-methyl-1H-pyrazol-4-yl)pyrazolo[1,5-a]pyrazin-4-yl)oxy)cyclobutyl)acrylamide

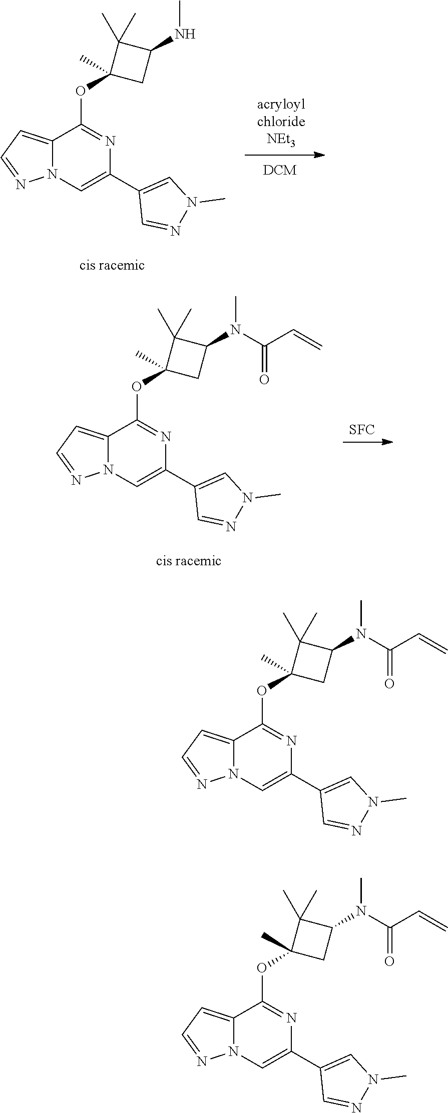 [SMILES: CN[C@H]1C[C@@](C)(Oc2nc(-c3cnn(C)c3)cn3nccc23)C1(C)C ; C=CC(=O)N(C)[C@H]1C[C@@](C)(Oc2nc(-c3cnn(C)c3)cn3nccc23)C1(C)C ; "SFC" ; C=CC(=O)N(C)[C@H]1C[C@@](C)(Oc2nc(-c3cnn(C)c3)cn3nccc23)C1(C)C ; C=CC(=O)N(C)[C@@H]1C[C@](C)(Oc2nc(-c3cnn(C)c3)cn3nccc23)C1(C)C]

To an ice-cooled solution of racemic (1S,3R)—N,2,2,3-tetramethyl-3-((6-(1-methyl-1H-pyrazol-4-yl)pyrazolo[1,5-a]pyrazin-4-yl)oxy)cyclobutan-1-amine (1.75 g, 5.14 mmol) in DCM (150 mL) was added triethylamine (2.15 mL, 15.4 mmol), followed by acryloyl chloride (627 μL, 7.71 mmol) and the reaction was stirred at 0° C. for 15 mins. The reaction mixture was purified directly by silica gel column chromatography (from 0% to 100% [3:1 EtOAc/EtOH] in heptanes) to give racemic N-methyl-N-((1S,3R)-2,2,3-trimethyl-3-((6-(1-methyl-1H-pyrazol-4-yl)pyrazolo[1,5-a]pyrazin-4-yl)oxy)cyclobutyl)acrylamide as a white solid (1.15 g, 57% yield). ESI-MS (M+H)+: 395.4.

The racemic material was resolved by chiral SFC purification (CHIRALPAK AD-H 30×250 mm, 5 μm column using 40% MeOH in CO₂. Flow rate: 100 mL/min; ABPR 120 bar; MBPR 40 psi, column temperature 40° C.) to give:

### Example 140: First eluting peak, (Rt=2.41 min), (373 mg, 18% yield). ¹H NMR (500 MHz, DMSO-d₆) δ: 8.72 (s, 1H), 8.14 (s, 1H), 8.03-7.93 (m, 2H), 6.79 (br d, J=1.8 Hz, 2H), 6.21-6.02 (m, 1H), 5.75-5.60 (m, 1H), 4.15 (br s, 1H), 3.94-3.83 (m, 3H), 3.17 (d, J=5.5 Hz, 1H), 3.06-2.89 (m, 3H), 2.87-2.65 (m, 1H), 1.85-1.71 (m, 3H), 1.37-1.22 (m, 3H), 1.11-0.95 (m, 3H)

### Example 141: And the second eluting peak (Rt=3.94 min), (399 mg, 19% yield). ¹H NMR (500 MHz, DMSO-d₆) δ: 8.72 (s, 1H), 8.14 (s, 1H), 8.05-7.92 (m, 2H), 6.79 (br d, J=1.2 Hz, 2H), 6.12 (br s, 1H), 5.68 (br s, 1H), 4.15 (br s, 1H), 3.89 (s, 3H), 3.17 (d, J=4.9 Hz, 1H), 3.06-2.90 (m, 3H), 2.88-2.66 (m, 1H), 1.77 (br s, 3H), 1.31 (br s, 3H), 1.10-0.95 (m, 3H)

The absolute stereochemistry of the product in each peak was not assigned, but the cis configuration between the ether and the amide was confirmed by ¹H NMR NOESY experiments

### Example 142: N-methyl-N-((trans)-3-((6-(1-methyl-1H-pyrazol-4-yl)pyrazolo[1,5-a]pyrazin-4-yl)oxy)cyclopentyl)acrylamide

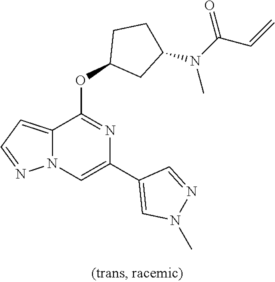 [SMILES: C=CC(=O)N(C)[C@H]1CC[C@H](Oc2nc(-c3cnn(C)c3)cn3nccc23)C1]

### 1. Synthesis of tert-butyl ((trans)-3-((6-(1-methyl-1H-pyrazol-4-yl)pyrazolo[1,5-a]pyrazin-4-yl)oxy)cyclopentyl)carbamate

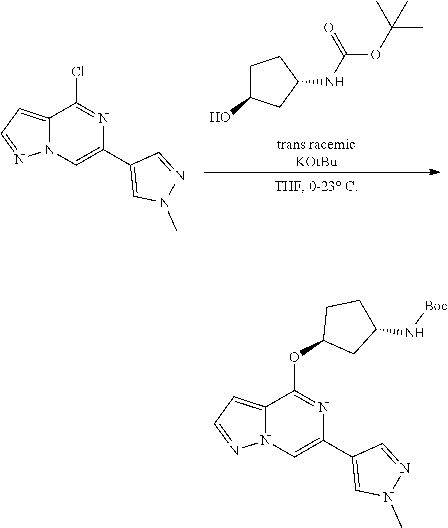 [SMILES: Cn1cc(-c2cn3nccc3c(Cl)n2)cn1 ; CC(C)(C)OC(=O)N[C@H]1CC[C@H](O)C1 ; "trans racemicKOtBu" ; Cn1cc(-c2cn3nccc3c(O[C@H]3CC[C@H](NC(=O)OC(C)(C)C)C3)n2)cn1]

A flask containing tert-butyl ((1,3-trans)-3-hydroxycyclopentyl)carbamate (250 mg, 1.24 mmol) in anhydrous THF (5 mL) was cooled in an ice water bath, then KOtBu (123 mg, 1.1 mmol) was added carefully in portions and the mixture stirred for 15 mins. 4-Chloro-6-(1-methyl-1H-pyrazol-4-yl)pyrazolo[1,5-a]pyrazine (250 mg, 1.07 mmol) was then added and the reaction allowed to warm to 23° C. After 30 mins, the reaction was quenched with slow addition of saturated aqueous sodium bicarbonate, then the biphasic mixture was extracted with EtOAc (3×). The combined organics were dried over MgSO₄, filtered and the filtrate concentrated under reduced pressure. The residue was purified by silica gel column chromatography (EtOAc in heptane) to give tert-butyl ((trans)-3-((6-(1-methyl-1H-pyrazol-4-yl)pyrazolo[1,5-a]pyrazin-4-yl)oxy)cyclopentyl)carbamate (375 mg, 88% yield). ESI-MS (M+H)⁺: 399.2.

### 2. Synthesis of (trans)-3-((6-(1-methyl-1H-pyrazol-4-yl)pyrazolo[1,5-a]pyrazin-4-yl)oxy)cyclopentan-1-amine dihydrochloride

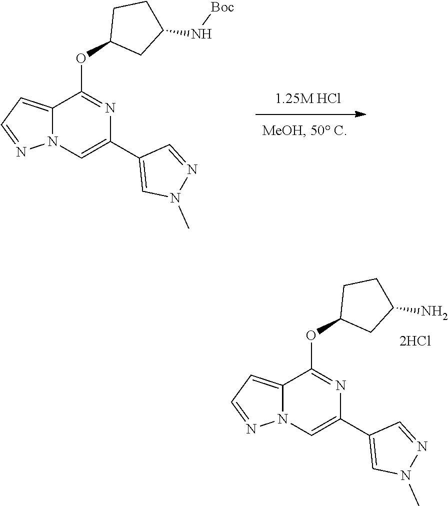 [SMILES: Cn1cc(-c2cn3nccc3c(O[C@H]3CC[C@H](NC(=O)OC(C)(C)C)C3)n2)cn1 ; Cn1cc(-c2cn3nccc3c(O[C@H]3CC[C@H](N)C3)n2)cn1]

To a vial containing tert-butyl ((trans)-3-((6-(1-methyl-1H-pyrazol-4-yl)pyrazolo[1,5-a]pyrazin-4-yl)oxy)cyclopentyl)carbamate (375 mg, 941 μmol) was added an HCl solution (1.25 M in MeOH, 5 mL) dropwise and the reaction was warmed to 50° C. After 2 h, the reaction was concentrated under reduced pressure to afford (trans)-3-((6-(1-methyl-1H-pyrazol-4-yl)pyrazolo[1,5-a]pyrazin-4-yl)oxy)cyclopentan-1-amine dihydrochloride (327 mg, crude), which was carried forward without further purification. ESI-MS (M+H)⁺: 299.0.

### 3. Synthesis of N-((trans)-3-((6-(1-methyl-1H-pyrazol-4-yl)pyrazolo[1,5-a]pyrazin-4-yl)oxy)cyclopentyl)acrylamide

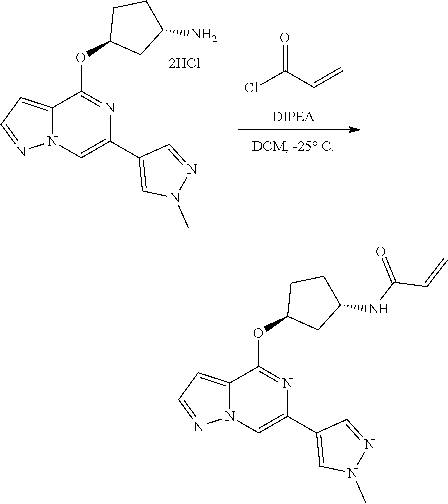

To a vial containing (trans)-3-((6-(1-methyl-1H-pyrazol-4-yl)pyrazolo[1,5-a]pyrazin-4-yl)oxy)cyclopentan-1-amine dihydrochloride (155 mg, 520 μmol) in anhydrous DCM (3 mL) was added DIPEA (0.27 mL, 1.56 mmol) dropwise at −25° C. After 5 mins, acryloyl chloride (0.04 mL, 520 μmol) was added dropwise and the reaction stirred for 3 mins. The reaction was quenched with slow addition of aqueous 1 M NaOH solution, the mixture was stirred at 23° C. for 1 h, the phases separated and the aqueous phase extracted with DCM (3×). The combined organics were washed with sat. aq. NaHCO₃ solution, then dried over anhydrous Na₂SO₄, filtered and the filtrate concentrated under reduced pressure. The residue was purified by silica gel column ([3:1 EtOAc:EtOH] in heptanes) to give N-((trans)-3-((6-(1-methyl-1H-pyrazol-4-yl)pyrazolo[1,5-a]pyrazin-4-yl)oxy)cyclopentyl)acrylamide (70 mg, 38% yield). ESI-MS (M+H)⁺: 353.1.

### 4. Synthesis of N-methyl-N-((trans)-3-((6-(1-methyl-1H-pyrazol-4-yl)pyrazolo[1,5-a]pyrazin-4-yl)oxy)cyclopentyl)acrylamide

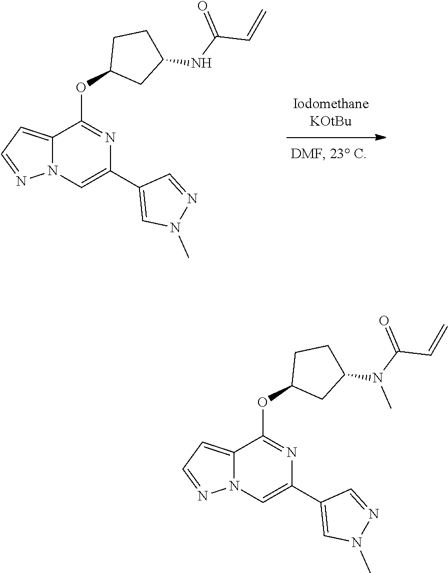 [SMILES: C=CC(=O)N[C@H]1CC[C@H](Oc2nc(-c3cnn(C)c3)cn3nccc23)C1 ; C=CC(=O)N(C)[C@H]1CC[C@H](Oc2nc(-c3cnn(C)c3)cn3nccc23)C1]

To a vial containing N-((trans)-3-((6-(1-methyl-1H-pyrazol-4-yl)pyrazolo[1,5-a]pyrazin-4-yl)oxy)cyclopentyl)acrylamide (50 mg, 142 μmol) in anhydrous DMF (1 mL) was added iodomethane (22 mL, 352 μmol), followed by KOtBu solution (1 M in THF, 284 μL) at RT and the reaction stirred for 30 mins. The reaction was diluted with DCM and purified directly by silica gel column ([3:1 EtOAc: EtOH] in heptane) to give N-methyl-N-((trans)-3-((6-(1-methyl-1H-pyrazol-4-yl)pyrazolo[1,5-a]pyrazin-4-yl)oxy)cyclopentyl)acrylamide (44 mg, 80% yield). ESI-MS (M+Na)⁺: 389.1. ¹H NMR (500 MHz, DMSO-d₆) δ=8.74 (s, 1H), 8.20 (s, 1H), 8.01 (d, J=3.1 Hz, 2H), 6.91-6.71 (m, 2H), 6.13-6.02 (m, 1H), 5.76-5.62 (m, 2H), 5.21-4.62 (m, 1H), 3.89 (s, 3H), 3.01-2.73 (m, 4H), 2.43-2.35 (m, 1H), 2.28-2.13 (m, 1H), 2.04-1.98 (m, 1H), 1.91-1.86 (m, 1H), 1.82-1.68 (m, 1H).

### Example 143: N-methyl-N-((cis)-3-((6-(1-methyl-1H-pyrazol-4-yl)pyrazolo[1,5-a]pyrazin-4-yl)oxy)cyclopentyl)acrylamide

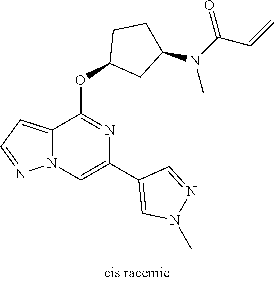 [SMILES: C=CC(=O)N(C)[C@@H]1CC[C@H](Oc2nc(-c3cnn(C)c3)cn3nccc23)C1]

N-Methyl-N-((cis)-3-((6-(1-methyl-1H-pyrazol-4-yl)pyrazolo[1,5-a]pyrazin-4-yl)oxy)cyclopentyl)acrylamide was obtained from tert-butyl ((cis)-3-hydroxycyclopentyl)carbamate and 4-chloro-6-(1-methyl-1H-pyrazol-4-yl)pyrazolo[1,5-a]pyrazine, following the 4 step procedure described in Example 142. ESI-MS (M+H)⁺: 367.1. ¹H NMR (500 MHz, DMSO-d₆) δ=8.74 (s, 1H), 8.21 (s, 1H), 8.03-7.99 (m, 2H), 6.95-6.69 (m, 2H), 6.17-6.03 (m, 1H), 5.70-5.63 (m, 2H), 5.18-4.55 (m, 1H), 3.88 (s, 3H), 2.99-2.72 (m, 4H), 2.50-2.39 (m, 2H), 2.03-1.97 (m, 1H), 1.91-1.81 (m, 2H).

### Examples 144 & 145: N-methyl-N-((1R,3S)-3-((6-(1-methyl-1H-pyrazol-4-yl)pyrazolo[1,5-a]pyrazin-4-yl)oxy)cyclopentyl)acrylamide and N-methyl-N-((1S,3R)-3-((6-(1-methyl-1H-pyrazol-4-yl)pyrazolo[1,5-a]pyrazin-4-yl)oxy)cyclopentyl)acrylamide

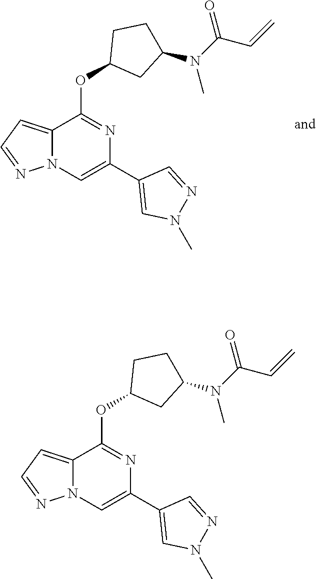 [SMILES: C=CC(=O)N(C)[C@@H]1CC[C@H](Oc2nc(-c3cnn(C)c3)cn3nccc23)C1 ; C=CC(=O)N(C)[C@H]1CC[C@@H](Oc2nc(-c3cnn(C)c3)cn3nccc23)C1]

### 1. Chiral resolution of N-methyl-N-((cis)-3-((6-(1-methyl-1H-pyrazol-4-yl)pyrazolo[1,5-a]pyrazin-4-yl)oxy)cyclopentyl)acrylamide

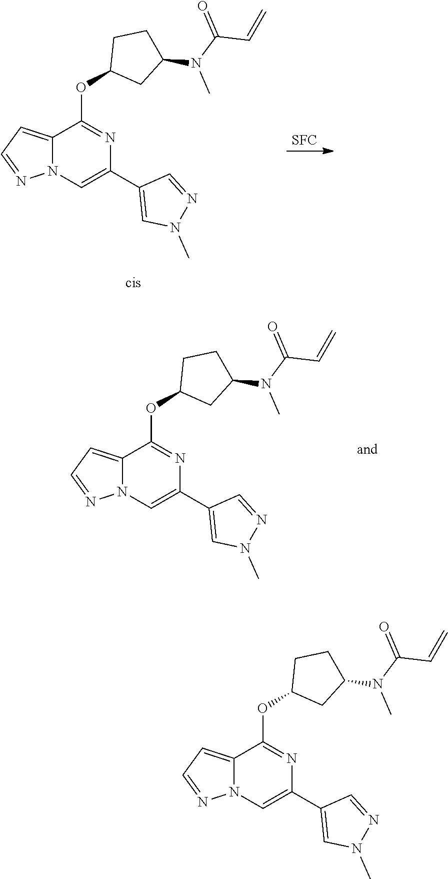 [SMILES: C=CC(=O)N(C)[C@@H]1CC[C@H](Oc2nc(-c3cnn(C)c3)cn3nccc23)C1 ; "SFC" ; C=CC(=O)N(C)[C@@H]1CC[C@H](Oc2nc(-c3cnn(C)c3)cn3nccc23)C1 ; C=CC(=O)N(C)[C@H]1CC[C@@H](Oc2nc(-c3cnn(C)c3)cn3nccc23)C1]

N-Methyl-N-((cis)-3-((6-(1-methyl-1H-pyrazol-4-yl)pyrazolo[1,5-a]pyrazin-4-yl)oxy)cyclopentyl)acrylamide (130 mg, 355 μmol) was resolved by chiral SFC purification (Chiralpak AD-H, 30×250 mm, 5 mm column using 40% MeOH in CO₂. Flow rate: 100 mL/min; ABPR 120 bar; MBPR 40 psi, column temperature 40° C.) to afford the following compounds that were concentrated to dryness then lyophilized to afford:

### Example 144: First eluting enantiomer (E1) Peak 1 (35 mg, 28%). Rt=2.88 min. ESI-MS (M+H)⁺: 367.1. H NMR (500 MHz, DMSO-d6) δ=8.75 (s, 1H), 8.21 (br s, 1H), 8.04-7.96 (m, 2H), 6.96-6.69 (m, 2H), 6.09 (br t, J=16.5 Hz, 1H), 5.71-5.62 (m, 2H), 5.18-4.59 (m, 1H), 3.88 (s, 3H), 3.00-2.82 (m, 3H), 2.49-2.41 (m, 1H), 2.13-2.00 (m, 2H), 1.92-1.75 (m, 3H)

### Example 145: Second eluting enantiomer (E2) (37 mg, 28% yield). Rt=3.37 min. ESI-MS (M+H)⁺: 367.1. ¹H NMR (500 MHz, DMSO-d₆) δ=8.75 (s, 1H), 8.21 (br s, 1H), 8.04-7.96 (m, 2H), 6.96-6.69 (m, 2H), 6.09 (br t, J=16.5 Hz, 1H), 5.71-5.62 (m, 2H), 5.18-4.59 (m, 1H), 3.88 (s, 3H), 3.00-2.82 (m, 3H), 2.49-2.41 (m, 1H), 2.13-2.00 (m, 2H), 1.92-1.75 (m, 3H)

### Example 146: N-(2,2-difluoroethyl)-N-((1S,3R)-3-((6-(1-methyl-1H-pyrazol-4-yl)pyrazolo[1,5-a]pyrazin-4-yl)oxy)cyclopentyl)acrylamide

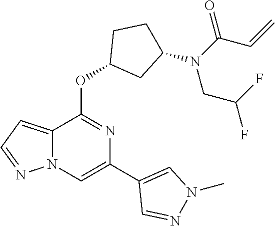 [SMILES: C=CC(=O)N(CC(F)F)[C@H]1CC[C@@H](Oc2nc(-c3cnn(C)c3)cn3nccc23)C1]

### 1. Synthesis of tert-butyl ((1S,3R)-3-((6-(1-methyl-1H-pyrazol-4-yl)pyrazolo[1,5-a]pyrazin-4-yl)oxy)cyclopentyl)carbamate

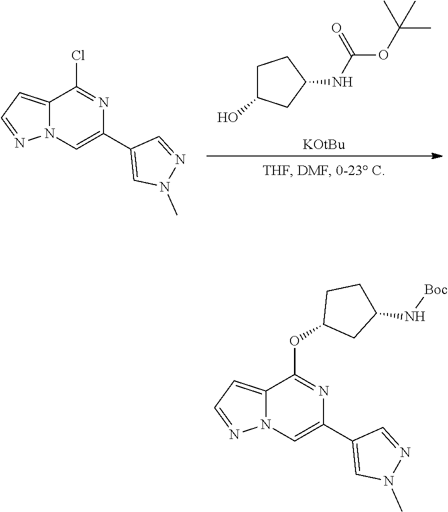 [SMILES: Cn1cc(-c2cn3nccc3c(Cl)n2)cn1 ; CC(C)(C)OC(=O)N[C@H]1CC[C@@H](O)C1 ; Cn1cc(-c2cn3nccc3c(O[C@@H]3CC[C@H](NC(=O)OC(C)(C)C)C3)n2)cn1 ; "KOtBu"]

A flask containing tert-butyl ((1S,3R)-3-hydroxycyclopentyl)carbamate (800 mg, 3.97 mmol) in anhydrous THF (10 mL) and DMF (2 mL) was cooled in an ice water bath, then 1 M KOtBu in THF (4.5 mL, 4.5 mmol) was added dropwise. After 15 mins, 4-chloro-6-(1-methyl-1H-pyrazol-4-yl)pyrazolo[1,5-a]pyrazine (1 g, 4.28 mmol) was added in portions and the mixture was then allowed to warm to 23° C. After 30 mins, the reaction was quenched with slow addition of saturated aqueous sodium bicarbonate, the layers separated and the aqueous phase extracted with EtOAc (3×). The combined organics were dried over anhydrous Na₂SO₄, filtered and concentrated under reduced pressure. The residue was loaded onto a silica gel column and purified (EtOAc in heptane) to give tert-butyl ((1S,3R)-3-((6-(1-methyl-1H-pyrazol-4-yl)pyrazolo[1,5-a]pyrazin-4-yl)oxy)cyclopentyl)carbamate (1.58 g, 100% yield). ESI-MS (M+H)⁺: 399.2.

### 2. Synthesis of (1S,3R)-3-((6-(1-methyl-1H-pyrazol-4-yl)pyrazolo[1,5-a]pyrazin-4-yl)oxy)cyclopentan-1-amine dihydrochloride

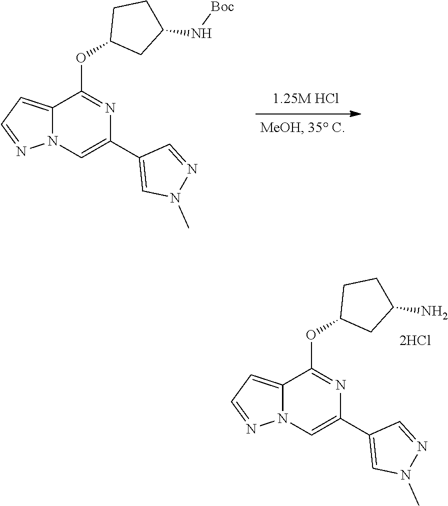 [SMILES: Cn1cc(-c2cn3nccc3c(O[C@@H]3CC[C@H](NC(=O)OC(C)(C)C)C3)n2)cn1 ; Cn1cc(-c2cn3nccc3c(O[C@@H]3CC[C@H](N)C3)n2)cn1]

To a flask containing tert-butyl ((1S,3R)-3-((6-(1-methyl-1H-pyrazol-4-yl)pyrazolo[1,5-a]pyrazin-4-yl)oxy)cyclopentyl)carbamate (3 g, 7.5 mmol) was added MeOH (25 mL), followed by HCl (1.25 M in MeOH, 30 mL) dropwise and the reaction warmed to 35° C. After 19 h, additional HCl in MeOH (1.25 M, 30 mL) was added and heating continued at 35° C. After a total of 40 h, the reaction was concentrated under reduced pressure to a low volume then diluted with EtOAc. The heterogenous mixture was concentrated under reduced pressure to afford (1S,3R)-3-((6-(1-methyl-1H-pyrazol-4-yl)pyrazolo[1,5-a]pyrazin-4-yl)oxy)cyclopentan-1-amine dihydrochloride (2.7 g, crude) as a white solid, which was carried forward without further purification. ESI-MS (M+H)⁺: 299.1.

### 3. Synthesis of N-(2,2-difluoroethyl)-N—((1S,3R)-3-((6-(1-methyl-1H-pyrazol-4-yl)pyrazolo[1,5-a]pyrazin-4-yl)oxy)cyclopentyl)acrylamide

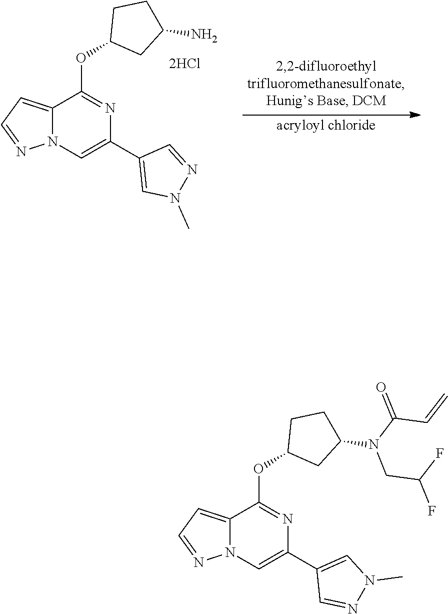 [SMILES: Cn1cc(-c2cn3nccc3c(O[C@@H]3CC[C@H](N)C3)n2)cn1 ; C=CC(=O)N(CC(F)F)[C@H]1CC[C@@H](Oc2nc(-c3cnn(C)c3)cn3nccc23)C1]

To a vial containing (1S,3R)-3-((6-(1-methyl-1H-pyrazol-4-yl)pyrazolo[1,5-a]pyrazin-4-yl)oxy)cyclopentan-1-amine dihydrochloride (50 mg, 149 μmol) in anhydrous DCM (5 mL) was added Hunig's Base (0.13 mL, 747 μmol) dropwise, followed by 2,2-difluoroethyl trifluoromethanesulfonate (32 mg, 149 μmol) at 23° C. After 18 h, additional 2,2-difluoroethyl trifluoromethanesulfonate (32 mg, 149 μmol) was added and the mixture was heated to 40° C. After 4 h, the reaction was cooled to 23° C., then acryloyl chloride (12 μL, 149 μmol) was added. After 10 mins, the reaction was quenched with the addition of aqueous 1 M NaOH solution. The mixture was stirred at 23° C. for 1 h, then the biphasic mixture was extracted with DCM (3×). The combined organics were washed with saturated aq. NaHCO₃ solution (2×), the organic layer was separated then dried over anhydrous Na₂SO₄, filtered and concentrated under reduced pressure, the residue was purified by a silica gel column ([3:1 EtOAc:EtOH] in heptane) to give N-(2,2-difluoroethyl)-N-((1S,3R)-3-((6-(1-methyl-1H-pyrazol-4-yl)pyrazolo[1,5-a]pyrazin-4-yl)oxy)cyclopentyl)acrylamide (44 mg, 69% yield). ESI-MS (M+H)⁺: 417.1. ¹H NMR (500 MHz, DMSO-d₆) δ: 8.76 (s, 1H), 8.22 (s, 1H), 8.05-8.01 (m, 2H), 7.03-6.71 (m, 2H), 6.35-6.08 (m, 2H), 5.82-5.60 (m, 2H), 4.69 (quin, J=8.5 Hz, 1H), 4.06-3.84 (m, 4H), 3.81-3.70 (m, 1H), 2.60-2.53 (m, 1H), 2.08-1.99 (m, 2H), 1.99-1.87 (m, 3H).

### Example 147: N-(2,2-difluoroethyl)-N-((1S,3R)-3-((6-(1-methyl-1H-pyrazol-4-yl)pyrazolo[1,5-a]pyrazin-4-yl)oxy)cyclopentyl)acrylamide

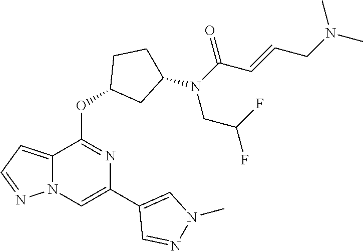 [SMILES: CN(C)C/C=C/C(=O)N(CC(F)F)[C@H]1CC[C@@H](Oc2nc(-c3cnn(C)c3)cn3nccc23)C1]

### 1. Synthesis of (E)-N-(2,2-difluoroethyl)-4-(dimethylamino)-N—((1S,3R)-3-((6-(1-methyl-1H-pyrazol-4-yl)pyrazolo[1,5-a]pyrazin-4-yl)oxy)cyclopentyl)but-2-enamide

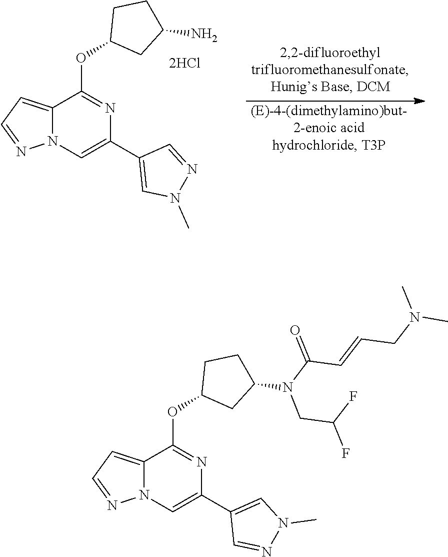 [SMILES: Cn1cc(-c2cn3nccc3c(O[C@@H]3CC[C@H](N)C3)n2)cn1 ; CN(C)C/C=C/C(=O)N(CC(F)F)[C@H]1CC[C@@H](Oc2nc(-c3cnn(C)c3)cn3nccc23)C1]

To a vial containing (1S,3R)-3-((6-(1-methyl-1H-pyrazol-4-yl)pyrazolo[1,5-a]pyrazin-4-yl)oxy)cyclopentan-1-amine dihydrochloride (Step 2, Example 146, 50 mg, 149 μmol) in anhydrous DCM (5 mL) was added Hunig's Base (0.13 mL, 747 μmol) dropwise, followed by 2,2-difluoroethyl trifluoromethanesulfonate (32 mg, 149 μmol) at 23° C. After 18 h, additional 2,2-difluoroethyl trifluoromethanesulfonate (32 mg, 149 μmol) was added and the mixture was heated to 40° C. After 4 h, the reaction was cooled to 23° C., then (E)-4-(dimethylamino)but-2-enoic acid hydrochloride (50 mg, 299 μmol) and 50% T3P in DMF (190 mg, 299 μmol) were added. The mixture was heated to 40° C. After 19 h, the reaction was cooled to RT then loaded onto a silica gel column and purified ([3:1 EtOAc:EtOH] in heptane) to give (E)-N-(2,2-difluoroethyl)-4-(dimethylamino)-N-((1S,3R)-3-((6-(1-methyl-1H-pyrazol-4-yl)pyrazolo[1,5-a]pyrazin-4-yl)oxy)cyclopentyl)but-2-enamide (28 mg, 39% yield). ESI-MS (M+H)⁺: 474.2. ¹H NMR (500 MHz, DMSO-d₆) δ: 8.75 (s, 1H), 8.20 (s, 1H), 8.04-8.00 (m, 2H), 7.37-6.65 (m, 4H), 6.57-6.53 (m, 1H), 6.34-6.05 (m, 1H), 5.70-5.59 (m, 1H), 4.72-4.64 (m, 1H), 3.88 (s, 3H), 3.77-3.65 (m, 1H), 3.03-3.00 (m, 2H), 2.58-2.53 (m, 1H), 2.15 (s, 3H), 2.14 (s, 3H), 2.08-1.99 (m, 2H), 1.91-1.85 (m, 2H).

### Example 148: (2S,3R)-3-methyl-N-((1S,3R)-3-((6-(1-methyl-1H-pyrazol-4-yl)pyrazolo[1,5-a]pyrazin-4-yl)oxy)cyclopentyl)oxirane-2-carboxamide and (2R,3S)-3-methyl-N-((1S,3R)-3-((6-(1-methyl-1H-pyrazol-4-yl)pyrazolo[1,5-a]pyrazin-4-yl)oxy)cyclopentyl)oxirane-2-carboxamide

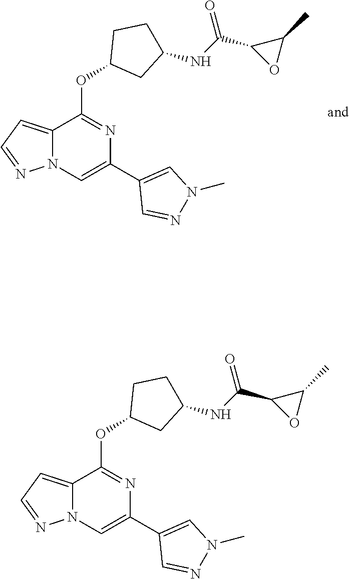 [SMILES: C[C@H]1O[C@@H]1C(=O)N[C@H]1CC[C@@H](Oc2nc(-c3cnn(C)c3)cn3nccc23)C1 ; C[C@@H]1O[C@H]1C(=O)N[C@H]1CC[C@@H](Oc2nc(-c3cnn(C)c3)cn3nccc23)C1]

### 1. Synthesis of (trans)-3-methyl-N-((1S,3R)-3-((6-(1-methyl-1H-pyrazol-4-yl)pyrazolo[1,5-a]pyrazin-4-yl)oxy)cyclopentyl)oxirane-2-carboxamide

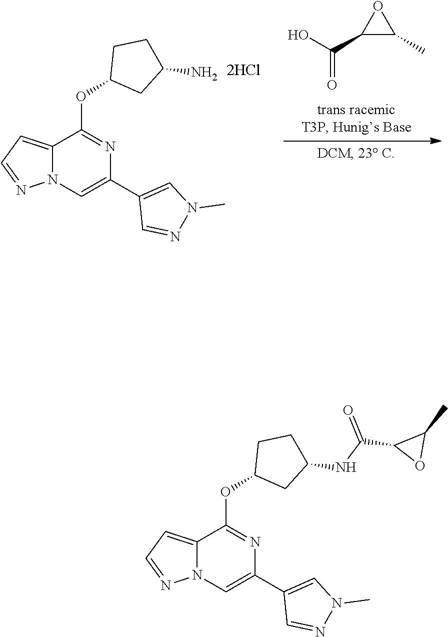

To a vial containing (1S,3R)-3-((6-(1-methyl-1H-pyrazol-4-yl)pyrazolo[1,5-a]pyrazin-4-yl)oxy)cyclopentan-1-amine dihydrochloride (Step 2, Example 146, 640 mg, 1.91 mmol) was added DCM (10 mL), Hunig's base (1.7 mL, 9.56 mmol) and (trans)-3-methyloxirane-2-carboxylic acid (293 mg, 2.87 mmol) at RT. 50% T3P in DMF (2.43 g, 3.82 mmol) was added dropwise and the reaction stirred for 30 mins. The reaction was loaded onto a silica gel column and purified ([3:1 EtOAc:EtOH] in heptane) to give (trans)-3-methyl-N-((1S,3R)-3-((6-(1-methyl-1H-pyrazol-4-yl)pyrazolo[1,5-a]pyrazin-4-yl)oxy)cyclopentyl)oxirane-2-carboxamide (650 mg, 89% yield). ESI-MS (M+H)⁺: 383.1.

### 2. Synthesis of (2S,3R) and (2R,3S)-3-methyl-N-((1S,3R)-3-((6-(1-methyl-1H-pyrazol-4-yl)pyrazolo[1,5-a]pyrazin-4-yl)oxy)cyclopentyl)oxirane-2-carboxamide

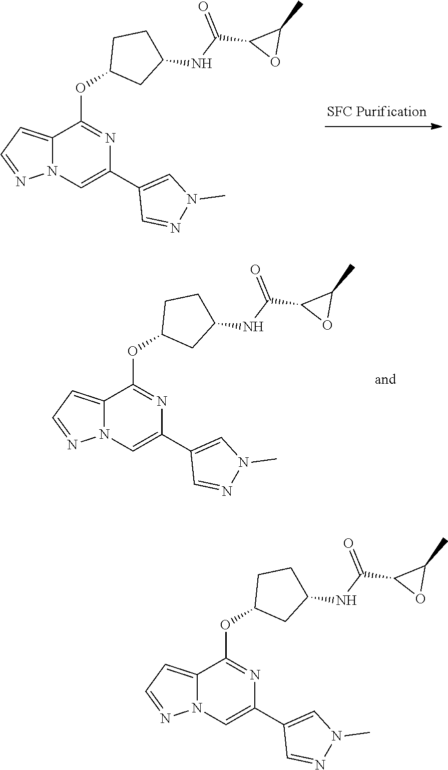 [SMILES: C[C@H]1O[C@@H]1C(=O)N[C@H]1CC[C@@H](Oc2nc(-c3cnn(C)c3)cn3nccc23)C1 ; C[C@H]1O[C@@H]1C(=O)N[C@H]1CC[C@@H](Oc2nc(-c3cnn(C)c3)cn3nccc23)C1 ; C[C@H]1O[C@@H]1C(=O)N[C@H]1CC[C@@H](Oc2nc(-c3cnn(C)c3)cn3nccc23)C1]

(trans)-3-Methyl-N-((1S,3R)-3-((6-(1-methyl-1H-pyrazol-4-yl)pyrazolo[1,5-a]pyrazin-4-yl)oxy)cyclopentyl)-oxirane-2-carboxamide (650 mg, 1.70 mmol) was resolved by chiral SFC purification (Chiralpak OX—H, 30×250 mm, 5 mm column using 30% MeOH in CO₂. Flow rate: 100 mL/min; ABPR 120 bar; MBPR 40 psi, column temperature 40° C.) to afford the following compounds that were concentrated to dryness, then lyophilized.

The first eluting peak, (163 mg, 25%, Rt=3.02 min, 100% ee) and the second eluting peak (Example 148, 163 mg, 25%, Rt=3.34 min, 97.68% ee). LCMS m/z=383.3 (M+H)⁺. ¹H NMR (500 MHz, DMSO-d₆) δ: 8.74 (s, 1H), 8.21 (s, 1H), 8.05-8.00 (m, 3H), 6.83 (d, J=1.5 Hz, 1H), 5.62-5.56 (m, 1H), 4.17 (sxt, J=7.3 Hz, 1H), 3.88 (s, 3H), 3.13 (d, J=1.8 Hz, 1H), 3.06 (dq, J=2.0, 5.1 Hz, 1H), 2.57-2.51 (m, 1H), 2.17-2.08 (m, 1H), 2.01-1.91 (m, 2H), 1.82-1.69 (m, 2H), 1.26 (d, J=5.2 Hz, 3H).

### Example 149: rac-N-methyl-N-((1R,3S)-3-((6-(1-methyl-1H-pyrazol-4-yl)pyrazolo[1,5-a]pyrazin-4-yl)oxy)cyclopentyl)cyclobut-1-ene-1-carboxamide

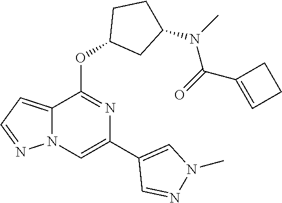 [SMILES: CN(C(=O)C1=CCC1)[C@H]1CC[C@@H](Oc2nc(-c3cnn(C)c3)cn3nccc23)C1]

### 1. Synthesis of rac-tert-butyl methyl((1R,3S)-3-((6-(1-methyl-1H-pyrazol-4-yl)pyrazolo[1,5-a]pyrazin-4-yl)oxy)cyclopentyl)carbamate

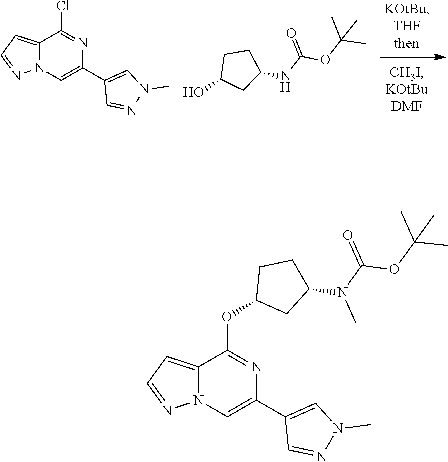

To a solution of 4-chloro-6-(1-methyl-1H-pyrazol-4-yl)pyrazolo[1,5-a]pyrazine (Intermediate A, 50 mg, 214 μmol) in THF (2 mL) was added rac-tert-butyl ((1R,3S)-3-hydroxycyclopentyl)carbamate (45 mg, 224 μmol), followed by a solution of KOtBu (1M, 250 μmol, 250 μL). The reaction mixture was stirred at ambient temperature for 30 mins before iodomethane (37 μL, 599 μmol) and an additional portion of a KOtBu solution (1M, 600 μmol, 600 μL) were added sequentially. DMF (1 mL) was added to solubilize the heterogeneous mixture and the reaction was stirred at ambient temperature for 30 mins. At this time, additional portions of iodomethane (85 mg, 599 μmol, 37 μL) and KOtBu solution (1M, 600 μmol, 600 μL) were added. After 30 more mins, a saturated, aqueous NaHCO₃ solution (10 mL) was added, followed by MTBE (10 mL). The layers were separated and the aqueous phase was extracted with MTBE (2×10 mL). The combined organic phase was concentrated in vacuo and purified by silica gel column chromatography (from 0 to 50% EtOAc in heptanes) to give rac-tert-butyl methyl((1R,3S)-3-((6-(1-methyl-1H-pyrazol-4-yl)pyrazolo[1,5-a]pyrazin-4-yl)oxy)cyclopentyl)carbamate (48 mg, 54% yield). ESI-MS (M+H)⁺: 413.2.

### 2. Synthesis of rac-(1R,3S)—N-methyl-3-((6-(1-methyl-1H-pyrazol-4-yl)pyrazolo[1,5-a]pyrazin-4-yl)oxy)cyclopentan-1-amine hydrochloride

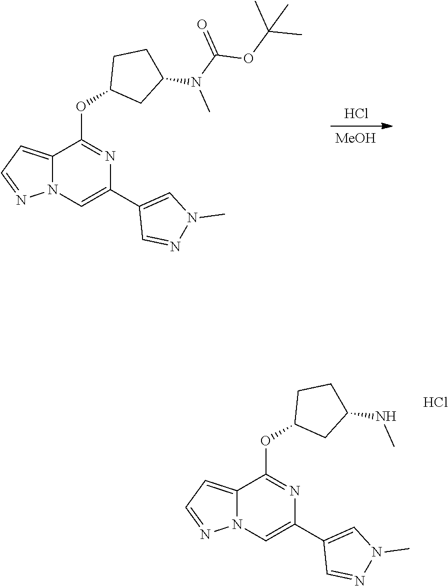 [SMILES: "MeOH" ; CN(C(=O)OC(C)(C)C)[C@H]1CC[C@@H](Oc2nc(-c3cnn(C)c3)cn3nccc23)C1 ; Cl ; CN[C@H]1CC[C@@H](Oc2nc(-c3cnn(C)c3)cn3nccc23)C1 ; Cl]

To a solution of rac-tert-butyl methyl((1R,3S)-3-((6-(1-methyl-1H-pyrazol-4-yl)pyrazolo[1,5-a]pyrazin-4-yl)oxy)cyclopentyl)carbamate (2.08 g, 5.03 mmol) in MeOH (25 mL) was added an HCl solution (1.25M in MeOH, 20 mL). The reaction mixture was stirred for 3 d at ambient temperature and id at 35° C. The reaction mixture was cooled to RT and concentrated. EtOAc was added and the mixture was concentrated to dryness to give rac-(1R,3S)—N-methyl-3-((6-(1-methyl-1H-pyrazol-4-yl)pyrazolo[1,5-a]pyrazin-4-yl)oxy)cyclopentan-1-amine hydrochloride (1.75 g, crude), which was carried forward without further purification. ESI-MS (M+H)⁺: 313.1.

### 3. Synthesis of rac-N-methyl-N—((1R,3S)-3-((6-(1-methyl-1H-pyrazol-4-yl)pyrazolo[1,5-a]pyrazin-4-yl)oxy)cyclopentyl)cyclobut-1-ene-1-carboxamide

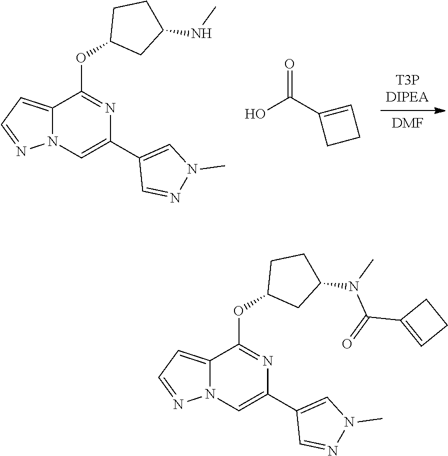

To a solution of rac-(1R,3S)—N-methyl-3-((6-(1-methyl-1H-pyrazol-4-yl)pyrazolo[1,5-a]pyrazin-4-yl)oxy)cyclopentan-1-amine hydrochloride (100 mg, 287 μmol) and cyclobut-1-ene-1-carboxylic acid (42 mg, 430 μmol) in dry DMF (1 mL) was added T3P (365 mg, 573 μmol, 385 μL, 50 wt. % in DMF) and DIPEA (200 μL, 1.15 mmol) and the reaction was stirred at 40° C. for 16 h. The mixture was diluted with EtOAc, then water was added carefully and the phases separated. The aqueous phase was extracted with EtOAc, the combined organic phases were washed with brine, dried (MgSO₄), filtered, and concentrated. The residual material was purified on Waters XSelect CSH Prep C18 column (5 um OBD 19×100 mm, purification gradient: 5-60%, purification modifier: NH₄OH) to give rac-N-methyl-N-((1R,3S)-3-((6-(1-methyl-1H-pyrazol-4-yl)pyrazolo[1,5-a]pyrazin-4-yl)oxy)cyclopentyl)cyclobut-1-ene-1-carboxamide as a white solid (64.4 mg, 57% yield). ESI-MS (M+H)⁺: 393.0. ¹H NMR (500 MHz, DMSO-d₆) δ: 8.75 (d, J=1.2 Hz, 2H), 8.21 (s, 2H), 8.03-8.00 (m, 4H), 6.83 (d, J=2.4 Hz, 2H), 6.48 (s, 2H), 5.68-5.62 (m, 2H), 5.11-4.97 (m, 1H), 4.79 (m, 1H), 3.89 (s, 6H), 3.02 (m, 3H), 2.80 (m, 3H), 2.74 (m, 2H), 2.69 (m, 2H), 2.47-2.41 (m, 2H), 2.38 (m, 2H), 1.95-2.10 (m, 6H), 1.89 (m, 3H), 1.81 (m, 3H). *Peaks in ¹H-NMR spectrum split as a 1:1 mixture due to the presence of amide rotamers. Both rotamers are described.

### Example 150: rac-(E)-3-cyano-N-methyl-N-((1R,3S)-3-((6-(1-methyl-1H-pyrazol-4-yl)pyrazolo[1,5-a]pyrazin-4-yl)oxy)cyclopentyl)acrylamide

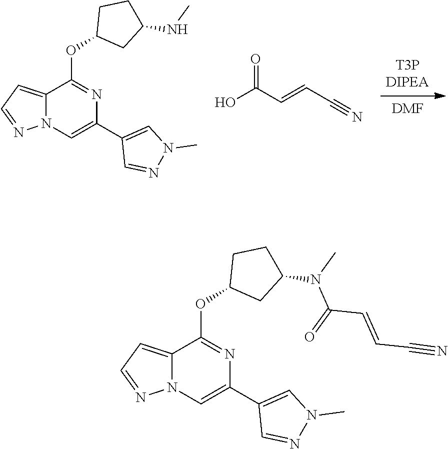

rac-(E)-3-cyano-N-methyl-N-((1R,3S)-3-((6-(1-methyl-1H-pyrazol-4-yl)pyrazolo[1,5-a]pyrazin-4-yl)oxy)cyclopentyl)acrylamide was obtained as a white solid (31.5 mg, 56% yield) from rac-(1R,3S)—N-methyl-3-((6-(1-methyl-1H-pyrazol-4-yl)pyrazolo[1,5-a]pyrazin-4-yl)oxy)cyclopentan-1-amine hydrochloride (step 2, Example 149) and (E)-3-cyanoacrylic acid following a similar procedure to that described in Example 149. ESI-MS (M+H)⁺: 392.0. ¹H NMR (500 MHz, DMSO-d₆)* δ: 8.75 (s, 2H), 8.26-8.17 (m, 3H), 8.04-8.00 (m, 4H), 7.87 (d, J=15.9 Hz, 1H), 7.66 (d, J=15.9 Hz, 1H), 6.85-6.82 (m, 2H), 6.51 (dd, J=15.9, 7.9 Hz, 1H), 5.71-5.59 (m, 2H), 5.05 (quin, J=8.7 Hz, 1H), 4.66-4.58 (m, 1H), 3.89 (d, J=2.4 Hz, 6H), 3.01 (s, 3H), 2.90-2.82 (m, 3H), 2.10-1.98 (m, 5H), 1.96-1.80 (m, 7H). *Peaks in ¹H-NMR spectrum split as a 1:1 mixture due to the presence of amide rotamers. Both rotamers are described.

### Example 151: 2-(methyl((1R,3S)-3-((6-(1-methyl-1H-pyrazol-4-yl)pyrazolo[1,5-a]pyrazin-4-yl)oxy)cyclopentyl)amino)acetonitrile

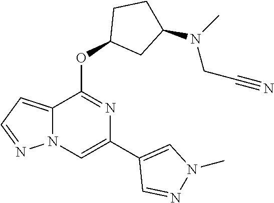 [SMILES: CN(CC#N)[C@@H]1CC[C@H](Oc2nc(-c3cnn(C)c3)cn3nccc23)C1]

### 1. Synthesis of tert-butyl ((1R,3S)-3-((6-(1-methyl-1H-pyrazol-4-yl)pyrazolo[1,5-a]pyrazin-4-yl)oxy)cyclopentyl)carbamate

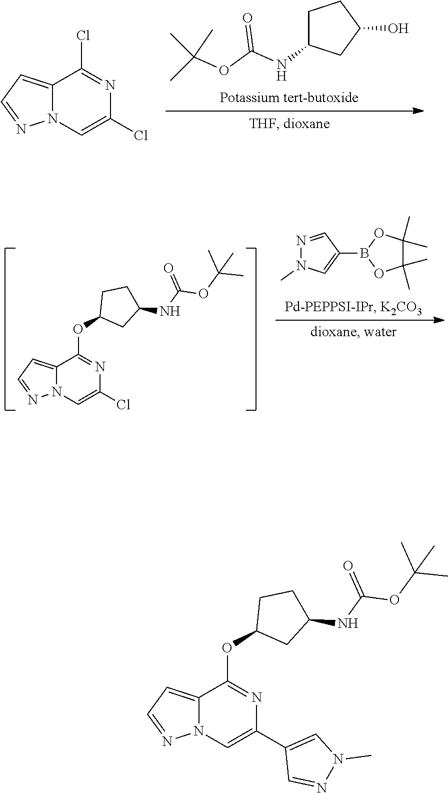

A flask containing tert-butyl ((1R,3S)-3-hydroxycyclopentyl)carbamate (11.8 g, 58.5 mmol) in anhydrous dioxane (100 mL) was cooled in an ice water bath, then 1 M KOtBu in THF (60 mL, 60 mmol) was added dropwise and the mixture stirred for 25 mins. 4,6-Dichloropyrazolo[1,5-a]pyrazine (10 g, 53.2 mmol) was added portionwise. The reaction mixture was stirred at ambient temperature for 30 mins before the reaction was degassed by bubbling N₂ through the reaction mixture. 1-Methyl-4-(4,4,5,5-tetramethyl-1,3,2-dioxaborolan-2-yl)pyrazole (33 g, 159 mmol), K₂CO₃ (22 g, 159 mmol), and Pd-PEPPSI™-IPr (700 mg, 1.03 mmol) were added, followed by water (50 mL) and the reaction was heated to 90° C. and stirred for 1 h. The reaction mixture was cooled to RT, diluted with water, then extracted with EtOAc (3×). The combined organics were dried over anhydrous Na₂SO₄, filtered and concentrated under reduced pressure. The residue was suspended in DCM (100 mL) and filtered through Celite®, washing through with additional DCM. The filtrate was concentrated under reduced pressure and the residue loaded onto a silica gel column and purified (EtOAc in heptane) to give tert-butyl ((1R,3S)-3-((6-(1-methyl-1H-pyrazol-4-yl)pyrazolo[1,5-a]pyrazin-4-yl)oxy)cyclopentyl)carbamate (29 g, 100% yield). ESI-MS (M+H)⁺: 399.2.

### 2. Synthesis of tert-butyl methyl((1R,3S)-3-((6-(1-methyl-1H-pyrazol-4-yl)pyrazolo[1,5-a]pyrazin-4-yl)oxy)cyclopentyl)carbamate

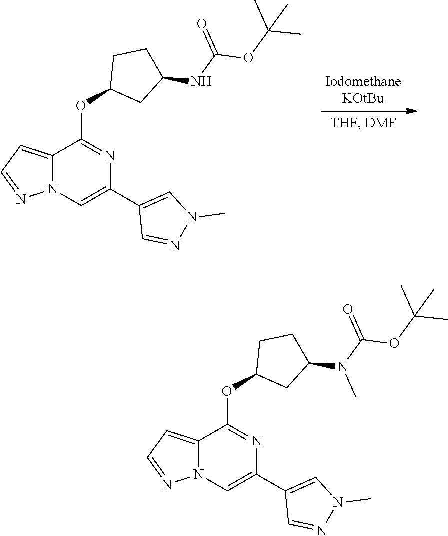 [SMILES: Cn1cc(-c2cn3nccc3c(O[C@H]3CC[C@@H](NC(=O)OC(C)(C)C)C3)n2)cn1 ; CN(C(=O)OC(C)(C)C)[C@@H]1CC[C@H](Oc2nc(-c3cnn(C)c3)cn3nccc23)C1]

A round bottom flask containing tert-butyl ((1R,3S)-3-((6-(1-methyl-1H-pyrazol-4-yl)pyrazolo[1,5-a]pyrazin-4-yl)oxy)cyclopentyl)carbamate (29 g, 54 mmol), anhydrous THF (100 mL), and anhydrous DMF (20 mL) was cooled in an ice water bath then iodomethane (4 mL, 64 mmol) was added dropwise. To the cooled mixture was added KOtBu (1 M, 60 mL) slowly, over 5 mins. The mixture was stirred for 10 mins before additional iodomethane (3.5 mL, 56 mmol) was added dropwise, followed by additional KOtBu (1 M, 60 mL). After 30 mins, the crude reaction mixture was concentrated to a low volume then diluted with MTBE and filtered through Celite®, washing through with MTBE. The filtrate was washed with water (100 mL) and the aqueous layer was extracted with MTBE (100 mL). The combined organic layers were concentrated to give a clear tan oil. The crude material was loaded onto a silica gel column and purified (EtOAc in heptane) to give tert-butyl methyl((1R,3S)-3-((6-(1-methyl-1H-pyrazol-4-yl)pyrazolo[1,5-a]pyrazin-4-yl)oxy)cyclopentyl)carbamate (19 g, 85% yield). ESI-MS (M+H)⁺: 413.2.

### 3. Synthesis of (1R,3S)—N-methyl-3-((6-(1-methyl-1H-pyrazol-4-yl)pyrazolo[1,5-a]pyrazin-4-yl)oxy)cyclopentan-1-amine hydrochloride

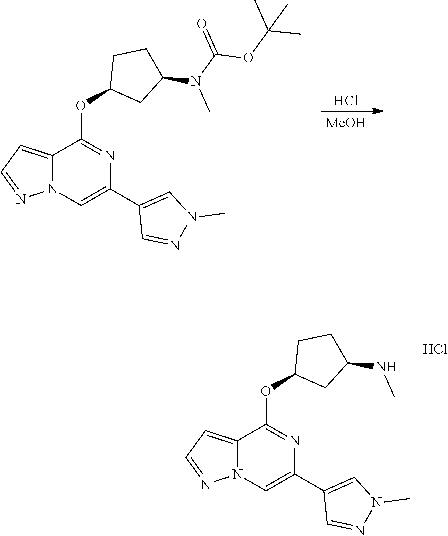 [SMILES: "MeOH" ; CN(C(=O)OC(C)(C)C)[C@@H]1CC[C@H](Oc2nc(-c3cnn(C)c3)cn3nccc23)C1 ; Cl ; CN[C@@H]1CC[C@H](Oc2nc(-c3cnn(C)c3)cn3nccc23)C1 ; Cl]

To a solution of tert-butyl methyl((1R,3S)-3-((6-(1-methyl-1H-pyrazol-4-yl)pyrazolo[1,5-a]pyrazin-4-yl)oxy)cyclopentyl)carbamate (19 g, 46 mmol) in MeOH (10 mL) was added an HCl solution (1.25M in MeOH, 200 mL) at 0° C. The reaction mixture was stirred for 19 h at ambient temperature and was concentrated to dryness to give (1R,3S)—N-methyl-3-((6-(1-methyl-1H-pyrazol-4-yl)pyrazolo[1,5-a]pyrazin-4-yl)oxy)cyclopentan-1-amine hydrochloride as a white solid (23 g, crude), which was carried forward without further purification. ESI-MS (M+H)⁺: 313.1.

### 4. Synthesis of 2-(methyl((1R,3S)-3-((6-(1-methyl-1H-pyrazol-4-yl)pyrazolo[1,5-a]pyrazin-4-yl)oxy)cyclopentyl)amino)acetonitrile

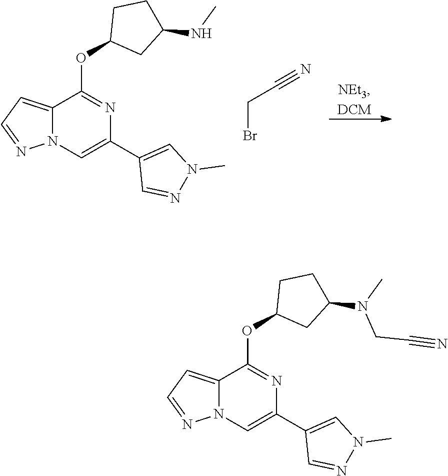

To a solution of (1R,3S)—N-methyl-3-((6-(1-methyl-1H-pyrazol-4-yl)pyrazolo[1,5-a]pyrazin-4-yl)oxy)cyclopentan-1-amine hydrochloride (50 mg, 143 μmol) and 2-bromoacetonitrile (34 mg, 287 μmol) in dry DCM (1 mL) was added Et₃N (73 mg, 717 μmol) and the reaction mixture stirred at ambient temperature for 16 h. The mixture was diluted with EtOAc, water was added and the phases separated. The aqueous phase was extracted with EtOAc and the combined organic phases were washed with brine, dried (MgSO₄), filtered, and concentrated. The residual material was purified by HLPC (Waters XSelect CSH Prep C18 column (5 um OBD 19×100 mm, purification gradient: 5-60%, purification modifier: NH₄OH)) to give 2-(methyl((1R,3S)-3-((6-(1-methyl-1H-pyrazol-4-yl)pyrazolo[1,5-a]pyrazin-4-yl)oxy)cyclopentyl)amino)acetonitrile as a white solid (16.8 mg, 29% yield). ESI-MS (M+H)⁺: 352.0. ¹H NMR (500 MHz, DMSO-d₆) δ: 8.74 (s, 1H), 8.21 (s, 1H), 8.06-7.96 (m, 2H), 6.81 (m, 1H), 5.66-5.58 (m, 1H), 3.89 (s, 3H), 3.82 (br s, 2H), 3.64-3.51 (m, 1H), 2.33 (s, 3H), 2.20-2.03 (m, 2H), 2.01-1.85 (m, 2H), 1.73-1.65 (m, 2H).

### Examples 152 & 153: 1-((1S,4S,5R)-5-((6-(1-methyl-1H-pyrazol-4-yl)pyrazolo[1,5-a]pyrazin-4-yl)oxy)-2-azabicyclo[2.2.1]heptan-2-yl)prop-2-en-1-one and 1-((1R,4R,5S)-5-((6-(1-methyl-1H-pyrazol-4-yl)pyrazolo[1,5-a]pyrazin-4-yl)oxy)-2-azabicyclo[2.2.1]heptan-2-yl)prop-2-en-1-one

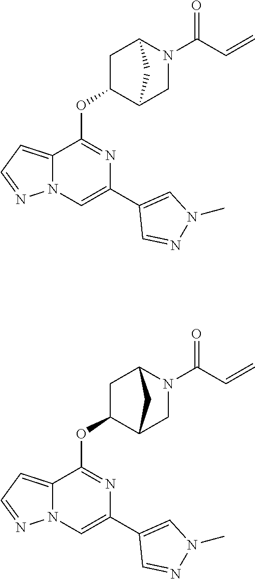 [SMILES: C=CC(=O)N1C[C@@H]2C[C@H]1C[C@H]2Oc1nc(-c2cnn(C)c2)cn2nccc12 ; C=CC(=O)N1C[C@H]2C[C@@H]1C[C@@H]2Oc1nc(-c2cnn(C)c2)cn2nccc12]

### 1. Synthesis of racemic tert-butyl (1R,4R,5S)-5-((6-(1-methyl-1H-pyrazol-4-yl)pyrazolo[1,5-a]pyrazin-4-yl)oxy)-2-azabicyclo[2.2.1]heptane-2-carboxylate

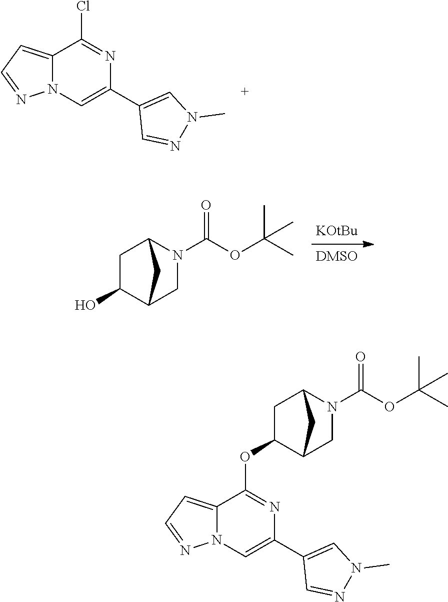

A vial was charged with racemic tert-butyl (1R,4R,5S)-5-hydroxy-2-azabicyclo[2.2.1]heptane-2-carboxylate (146 mg, 685 μmol) and THF (1.5 mL). A KOtBu solution (1.0 M in THF, 1.03 mL) was added and the reaction was stirred for 5 mins, followed by the addition of solid 4-chloro-6-(1-methylpyrazol-4-yl)pyrazolo[1,5-a]pyrazine (Intermediate A, 160 mg, 685 μmol). DMSO (1 mL) was added, and the resulting suspension was stirred at ambient temperature for 15 mins. Water was added, followed by EtOAc. The layers were separated and the aqueous phase was extracted with EtOAc. The combined organic extracts were washed with brine, dried (MgSO₄), filtered, and concentrated. The residue was purified by silica-gel chromatography (0-100% EtOAc in heptanes) to give racemic tert-butyl (1R,4R,5S)-5-((6-(1-methyl-1H-pyrazol-4-yl)pyrazolo[1,5-a]pyrazin-4-yl)oxy)-2-azabicyclo[2.2.1]heptane-2-carboxylate as an off-white solid (131 mg, 47% yield). ESI-MS (M+H)⁺: 411.4.

### 2. Synthesis of racemic 4-(((1R,4R,5S)-2-azabicyclo[2.2.1]heptan-5-yl)oxy)-6-(1-methyl-1H-pyrazol-4-yl)pyrazolo[1,5-a]pyrazine

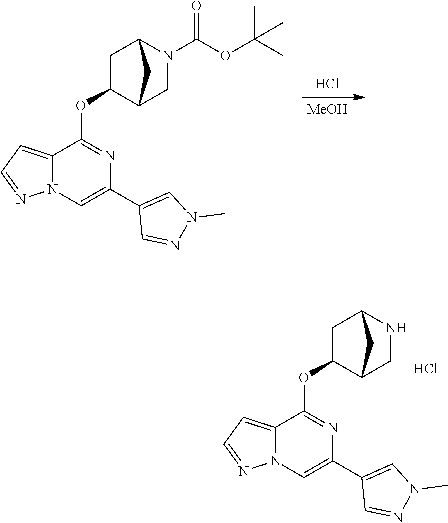 [SMILES: "MeOH" ; Cn1cc(-c2cn3nccc3c(O[C@H]3C[C@H]4C[C@@H]3CN4C(=O)OC(C)(C)C)n2)cn1 ; Cl ; Cn1cc(-c2cn3nccc3c(O[C@H]3C[C@H]4C[C@@H]3CN4)n2)cn1 ; Cl]

A suspension of racemic tert-butyl (1R,4R,5S)-5-((6-(1-methyl-1H-pyrazol-4-yl)pyrazolo[1,5-a]pyrazin-4-yl)oxy)-2-azabicyclo[2.2.1]heptane-2-carboxylate (130 mg, 317 μmol) in dry MeOH (1 mL) was treated with an HCl solution (4 M in dioxane, 554 μL). The resulting solution was stirred at RT for 1 h and was concentrated under vacuum to give racemic 4-(((1R,4R,5S)-2-azabicyclo[2.2.1]heptan-5-yl)oxy)-6-(1-methyl-1H-pyrazol-4-yl)pyrazolo[1,5-a]pyrazine hydrochloride as a pink solid (crude), which was carried forward without further purification. ESI-MS (M+H)⁺: 311.3

### 3. Synthesis of 1-((1S,4S,5R)-5-((6-(1-methyl-1H-pyrazol-4-yl)pyrazolo[1,5-a]pyrazin-4-yl)oxy)-2-azabicyclo[2.2.1]heptan-2-yl)prop-2-en-1-one and 1-((1R,4R,5S)-5-((6-(1-methyl-1H-pyrazol-4-yl)pyrazolo[1,5-a]pyrazin-4-yl)oxy)-2-azabicyclo[2.2.1]heptan-2-yl)prop-2-en-1-one

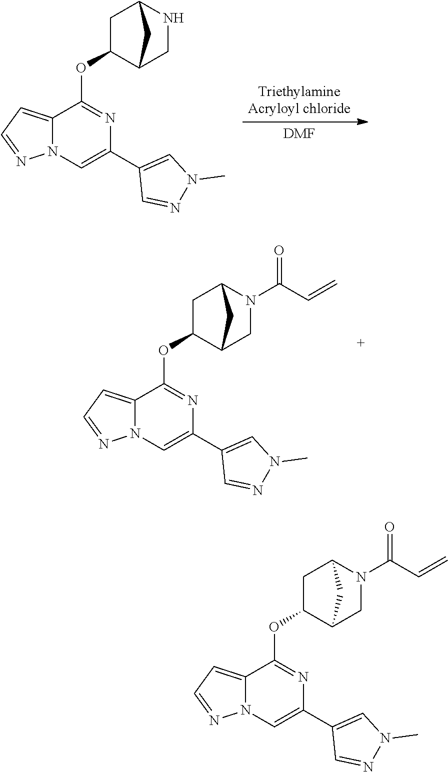

To a suspension of racemic 4-(((1R,4R,5S)-2-azabicyclo[2.2.1]heptan-5-yl)oxy)-6-(1-methyl-1H-pyrazol-4-yl)pyrazolo[1,5-a]pyrazine hydrochloride (109 mg, 314 μmol) in dry DMF (2 mL) at 0° C. under a N₂ atmosphere was added triethylamine (95 mg, 943 μmol), followed by acryloyl chloride (57 mg, 629 μmol). The resulting mixture was stirred at 0° C. for 30 min, quenched with saturated aqueous NaHCO₃ solution and extracted with EtOAc (5×). The combined organic extracts were washed with brine (5×), dried (MgSO₄), filtered, and concentrated under vacuum. The residue was purified by silica-gel chromatography (0-10% MeOH in DCM). An oil was obtained, which solidified with time. ESI-MS (M+H)⁺: 365.1. ¹H NMR (500 MHz, CDCl₃) δ: 8.23 (d, J=5.5 Hz, 1H), 7.89 (d, J=2.4 Hz, 1H), 7.84 (d, J=6.1 Hz, 1H), 7.79 (d, J=7.9 Hz, 1H), 6.74-6.70 (m, 1H), 6.55-6.33 (m, 2H), 5.79-5.67 (m, 1H), 5.42-5.28 (m, 1H), 4.50-4.42 (m, 1H), 4.85-4.42 (m, 1H), 3.98 (d, J=1.2 Hz, 3H), 3.64-3.52 (m, 1H), 3.42-3.30 (m, 1H), 3.07-3.00 (m, 1H), 2.53-2.34 (m, 1H), 2.11-1.98 (m, 1H), 1.98-1.87 (m, 1H), 1.87-1.71 (m, 1H).

This material was submitted to chiral separation on a LUX Cellulose-4 LC 30×250 mm, 5 um column using 40% MeOH/CO₂ (flow rate: 100 mL/min, ABPR 120 bar, MBPR 40 psi, column temp 40° C.) to give the first eluting peak (Example 152), as a white solid (40 mg, 35% yield, 100% ee) and the second eluting peak (Example 153), as a white solid (42 mg, 37% yield, 96.4% ee).

### Example 154: 1-(4-(methyl(6-(1-methyl-1H-pyrazol-4-yl)pyrazolo[1,5-a]pyridin-4-yl)amino)hexahydrocyclopenta[c]pyrrol-2(1H)-yl)prop-2-en-1-one

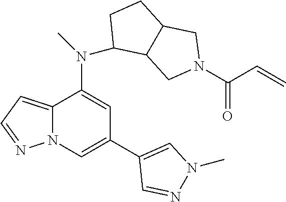 [SMILES: C=CC(=O)N1CC2CCC(N(C)c3cc(-c4cnn(C)c4)cn4nccc34)C2C1]

### 1. Synthesis of tert-butyl 4-((6-(1-methyl-1H-pyrazol-4-yl)pyrazolo[1,5-a]pyridin-4-yl)amino)hexahydrocyclopenta[c]pyrrole-2(1H)-carboxylate

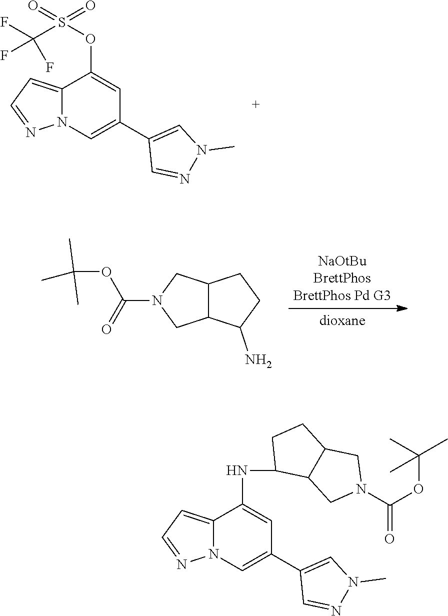 [SMILES: Cn1cc(-c2cc(OS(=O)(=O)C(F)(F)F)c3ccnn3c2)cn1 ; CC(C)(C)OC(=O)N1CC2CCC(N)C2C1 ; Cn1cc(-c2cc(NC3CCC4CN(C(=O)OC(C)(C)C)CC43)c3ccnn3c2)cn1]

A vial containing degassed dioxane (3 mL), tert-butyl 4-aminohexahydrocyclopenta[c]pyrrole-2(1H)-carboxylate (191 mg, 844 μmol), 6-(1-methyl-1H-pyrazol-4-yl)pyrazolo[1,5-a]pyridin-4-yl trifluoromethanesulfonate (Intermediate C, 200 mg, 578 μmol), BrettPhos (49 mg, 91 μmol), BrettPhos Pd G3 (90 mg, 99 μmol) and NaOtBu (192 mg, 2 mmol) was heated to 100° C. and stirred under N₂ for 2 h. The reaction mixture was cooled to RT, quenched with water and extracted with EtOAc (3×). The combined organic extracts were washed with brine (3×), dried (MgSO₄), filtered, and concentrated under vacuum. The residue was purified by silica-gel chromatography (0-100% EtOAc in heptanes) to give tert-butyl 4-((6-(1-methyl-1H-pyrazol-4-yl)pyrazolo[1,5-a]pyridin-4-yl)amino)hexahydrocyclopenta[c]pyrrole-2(1H)-carboxylate as a brown solid (57 mg, 24% yield). ESI-MS (M+H)⁺: 423.3.

### 2. Synthesis of tert-butyl 4-(methyl(6-(1-methyl-1H-pyrazol-4-yl)pyrazolo[1,5-a]pyridin-4-yl)amino)hexahydrocyclopenta[c]pyrrole-2(1H)-carboxylate

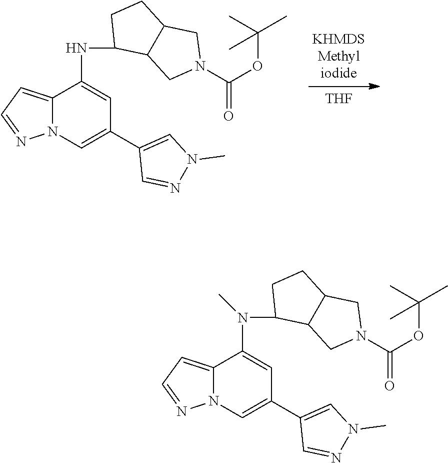 [SMILES: Cn1cc(-c2cc(NC3CCC4CN(C(=O)OC(C)(C)C)CC43)c3ccnn3c2)cn1 ; CN(c1cc(-c2cnn(C)c2)cn2nccc12)C1CCC2CN(C(=O)OC(C)(C)C)CC21 ; "THF"]

A KHMDS solution (0.5 M in toluene, 1.18 mL) was added to a solution of tert-butyl 4-((6-(1-methyl-1H-pyrazol-4-yl)pyrazolo[1,5-a]pyridin-4-yl)amino)hexahydrocyclopenta[c]pyrrole-2(1H)-carboxylate (50 mg, 118 μmol) in dry THF (1 mL) under N₂, cooled to 0° C. and the resulting mixture was stirred for 15 min at 0° C. Methyl iodide (84 mg, 592 μmol) was added and the reaction was stirred at 0° C. for 1 h. Water was added, followed by EtOAc, the phases were separated, and the aqueous phase was extracted with EtOAc (3×). The combined organic extracts were washed with brine, dried (MgSO₄), filtered, and concentrated under vacuum. The residue was purified by silica-gel chromatography (from 0-100% EtOAc in heptanes) to give tert-butyl 4-(methyl(6-(1-methyl-1H-pyrazol-4-yl)pyrazolo[1,5-a]pyridin-4-yl)amino)hexahydrocyclopenta[c]pyrrole-2(1H)-carboxylate as a brown solid (11 mg, 21% yield). ESI-MS (M+H)⁺: 437.3.

### 3. Synthesis of N-methyl-6-(1-methyl-1H-pyrazol-4-yl)-N-(octahydrocyclopenta[c]pyrrol-4-yl)pyrazolo[1,5-a]pyridin-4-amine hydrochloride

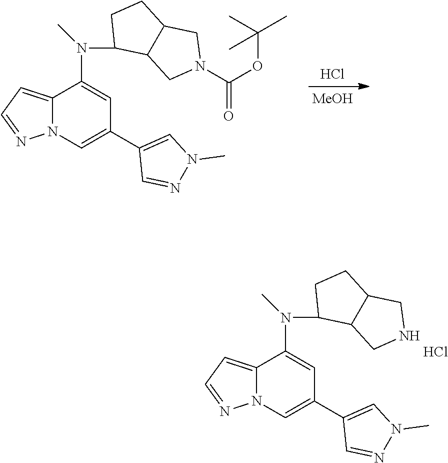 [SMILES: "MeOH" ; CN(c1cc(-c2cnn(C)c2)cn2nccc12)C1CCC2CN(C(=O)OC(C)(C)C)CC21 ; Cl ; CN(c1cc(-c2cnn(C)c2)cn2nccc12)C1CCC2CNCC21 ; Cl]

To a solution of tert-butyl 4-(methyl(6-(1-methyl-1H-pyrazol-4-yl)pyrazolo[1,5-a]pyridin-4-yl)amino)hexahydrocyclopenta[c]pyrrole-2(1H)-carboxylate (11 mg, 25 μmol) in dry MeOH (1 mL) was added an HCl solution (4 M in dioxane, 315 μL). The resulting mixture was stirred at ambient temperature for 1 h and was concentrated under vacuum to give N-(1,2,3,3a,4,5,6,6a-octahydrocyclopenta[c]pyrrol-4-yl)-N-methyl-6-(1-methylpyrazol-4-yl)pyrazolo[1,5-a]pyridin-4-amine hydrochloride as a pale, yellow solid (10 mg, crude), which was carried forward without further purification. ESI-MS (M+H)⁺: 337.2.

### 4. Synthesis of 1-(4-(methyl(6-(1-methyl-1H-pyrazol-4-yl)pyrazolo[1,5-a]pyridin-4-yl)amino)hexahydrocyclopenta[c]pyrrol-2(1H)-yl)prop-2-en-1-one

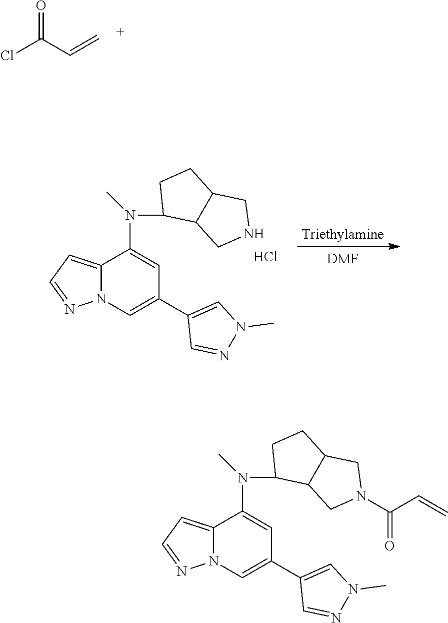

To a solution of N-(1,2,3,3a,4,5,6,6a-octahydrocyclopenta[c]pyrrol-4-yl)-N-methyl-6-(1-methylpyrazol-4-yl)pyrazolo[1,5-a]pyridin-4-amine hydrochloride (10 mg, 27 μmol) in dry DMF (1 mL) under N₂ at 0° C. was added triethylamine (7.4 mg, 73 μmol) followed by acryloyl chloride (6.6 mg, 73 μmol) and the reaction stirred at 0° C. for 1 h. The reaction was quenched with saturated NaHCO₃ solution and extracted with EtOAc (3×). The combined organic extracts were washed with brine (3×), dried (MgSO₄), filtered, and concentrated under vacuum. The residue was purified by preparative TLC (DCM/MeOH 95:5) to give 1-(4-(methyl(6-(1-methyl-1H-pyrazol-4-yl)pyrazolo[1,5-a]pyridin-4-yl)amino)hexahydrocyclopenta[c]pyrrol-2(1H)-yl)prop-2-en-1-one as a yellow film (2.4 mg, 24% yield). ESI-MS (M+H)⁺: 391.2. ¹H NMR (500 MHz, CDCl₃) δ: 8.29 (s, 1H), 7.88-7.85 (m, 1H), 7.74 (d, J=4.3 Hz, 1H), 7.62 (d, J=1.8 Hz, 1H), 6.53 (d, J=6.1 Hz, 1H), 6.51-6.27 (m, 3H), 5.71-5.59 (m, 1H), 4.18-4.06 (m, 1H), 3.98 (s, 3H), 3.81-3.57 (m, 3H), 3.51-3.38 (m, 1H), 2.91 (d, J=5.5 Hz, 3H), 2.89-2.75 (m, 1H), 2.15-2.03 (m, 2H), 2.01-1.90 (m, 1H), 1.58 (br s, 3H).

### Example 155 & 156: N-methyl-N-((cis)-3-((6-(1-((R)-tetrahydrofuran-3-yl)-1H-pyrazol-4-yl)pyrazolo[1,5-a]pyrazin-4-yl)oxy)cyclobutyl)acrylamide and N-methyl-N-((cis)-3-((6-(1-((S)-tetrahydrofuran-3-yl)-1H-pyrazol-4-yl)pyrazolo[1,5-a]pyrazin-4-yl)oxy)cyclobutyl)acrylamide

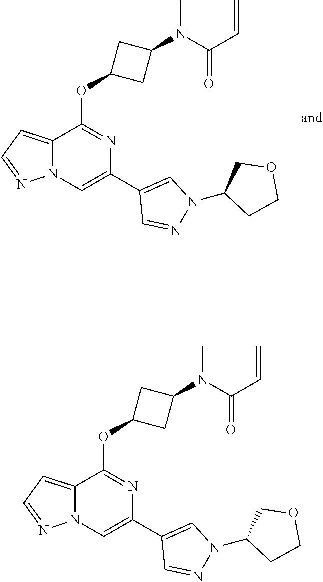 [SMILES: C=CC(=O)N(C)[C@H]1C[C@@H](Oc2nc(-c3cnn([C@@H]4CCOC4)c3)cn3nccc23)C1 ; C=CC(=O)N(C)[C@H]1C[C@@H](Oc2nc(-c3cnn([C@H]4CCOC4)c3)cn3nccc23)C1]

### 1. Synthesis of tert-butyl ((cis)-3-((6-chloropyrazolo[1,5-a]pyrazin-4-yl)oxy)cyclobutyl)(methyl)carbamate

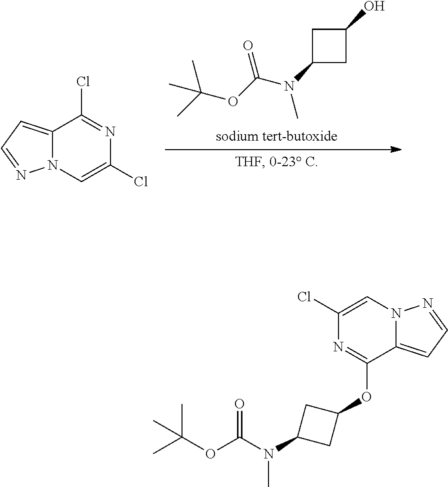 [SMILES: Clc1cn2nccc2c(Cl)n1 ; CN(C(=O)OC(C)(C)C)[C@H]1C[C@@H](O)C1 ; CN(C(=O)OC(C)(C)C)[C@H]1C[C@@H](Oc2nc(Cl)cn3nccc23)C1]

A vial containing cis tert-butyl N-(3-hydroxycyclobutyl)-N-methyl-carbamate (543 mg, 2.7 mmol) in anhydrous THF (5 mL) was cooled in an ice water bath, then NaOtBu (399 mg, 4.15 mmol) was added in portions to the cold mixture. After 15 mins, 4,6-dichloropyrazolo[1,5-a]pyrazine (488 mg, 2.6 mmol) was added in portions and the reaction allowed to warm to 23° C. and stirred for 75 mins. The reaction was quenched with water then the mixture was extracted with EtOAc (3×). The combined organics were dried over anhydrous Na₂SO₄, filtered and concentrated under reduced pressure. The residue was purified by silica gel column (10-50% EtOAc in heptane) to afford tert-butyl ((cis)-3-((6-chloropyrazolo[1,5-a]pyrazin-4-yl)oxy)cyclobutyl)(methyl)carbamate as a colorless oil (890 mg, yield: 97%) ESI-MS (M+H)⁺: 353.1.

### 2. Synthesis of cis tert-butyl methyl(3-((6-(1-(tetrahydrofuran-3-yl)-1H-pyrazol-4-yl)pyrazolo[1,5-a]pyrazin-4-yl)oxy)cyclobutyl)carbamate

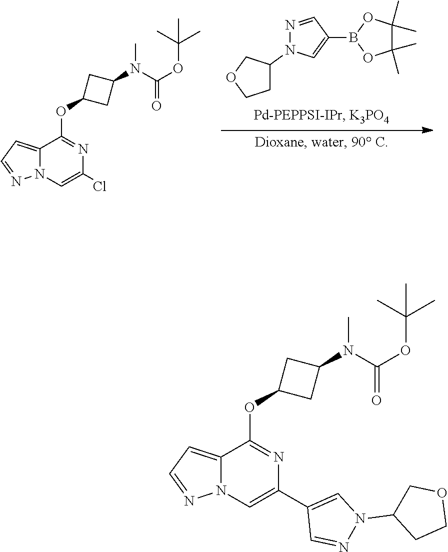 [SMILES: CN(C(=O)OC(C)(C)C)[C@H]1C[C@@H](Oc2nc(Cl)cn3nccc23)C1 ; CC1(C)OB(c2cnn(C3CCOC3)c2)OC1(C)C ; CN(C(=O)OC(C)(C)C)[C@H]1C[C@@H](Oc2nc(-c3cnn(C4CCOC4)c3)cn3nccc23)C1]

A vial containing tert-butyl ((cis)-3-((6-chloropyrazolo[1,5-a]pyrazin-4-yl)oxy)cyclobutyl)(methyl)carbamate (242 mg, 686 μmol), 1-tetrahydrofuran-3-yl-4-(4,4,5,5-tetramethyl-1,3,2-dioxaborolan-2-yl)pyrazole (374 mg, 1.4 mmol), K₃PO₄, 1M solution (2.0 mL), and Pd-PEPPSI™-IPr (98 mg, 144 μmol) in dioxane (4 mL) was degassed with N₂ and the reaction was stirred at 90° C. for 1 h. The reaction was carefully quenched with water, the phases separated, the aqueous layer extracted with EtOAc (3×) and the combined organic extracts dried over anhydrous Na₂SO₄. The filtrate was concentrated under reduced pressure, the residue purified by silica gel column (15-55% 3:1 EtOAc:EtOH in heptane) to give cis tert-butyl methyl(3-((6-(1-(tetrahydrofuran-3-yl)-1H-pyrazol-4-yl)pyrazolo[1,5-a]pyrazin-4-yl)oxy)cyclobutyl)carbamate (299 mg, yield: 96%). ESI-MS (M+H)⁺: 455.1. ¹H NMR (500 MHz, DMSO-d₆) d=8.79 (s, 1H), 8.31 (s, 1H), 8.07 (s, 1H), 8.02 (d, J=2.4 Hz, 1H), 6.85 (d, J=3.1 Hz, 1H), 5.12-5.06 (m, 2H), 4.49-4.08 (m, 1H), 4.04-3.98 (m, 2H), 3.92 (dd, J=3.7, 9.8 Hz, 1H), 3.88-3.83 (m, 1H), 2.82 (br s, 2H), 2.79 (s, 3H), 2.43-2.29 (m, 4H), 1.41 (s, 9H).

### 3. Synthesis of cis N-methyl-3-((6-(1-(tetrahydrofuran-3-yl)-1H-pyrazol-4-yl)pyrazolo[1,5-a]pyrazin-4-yl)oxy)cyclobutan-1-amine trifluoroacetate

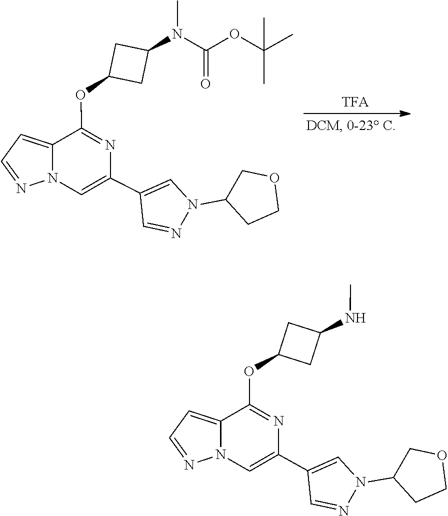 [SMILES: CN(C(=O)OC(C)(C)C)[C@H]1C[C@@H](Oc2nc(-c3cnn(C4CCOC4)c3)cn3nccc23)C1 ; CN[C@H]1C[C@@H](Oc2nc(-c3cnn(C4CCOC4)c3)cn3nccc23)C1]

A vial containing cis tert-butyl methyl(3-((6-(1-(tetrahydrofuran-3-yl)-1H-pyrazol-4-yl)pyrazolo[1,5-a]pyrazin-4-yl)oxy)cyclobutyl)carbamate (299 mg, 658 μmol) in DCM (2 mL) was cooled in an ice water bath, then TFA (0.45 mL, 5.88 mmol) was added dropwise and the reaction stirred for 4.5 h. The reaction was concentrated under reduced pressure to afford cis N-methyl-3-((6-(1-(tetrahydrofuran-3-yl)-1H-pyrazol-4-yl)pyrazolo[1,5-a]pyrazin-4-yl)oxy)cyclobutan-1-amine trifluoroacetate (308 mg, yield: 100%) as a colorless film that was used without purification. ESI-MS (M+H)⁺: 355.1.

### 4. Synthesis of cis N-methyl-N-(3-((6-(1-(tetrahydrofuran-3-yl)-1H-pyrazol-4-yl)pyrazolo[1,5-a]pyrazin-4-yl)oxy)cyclobutyl)acrylamide

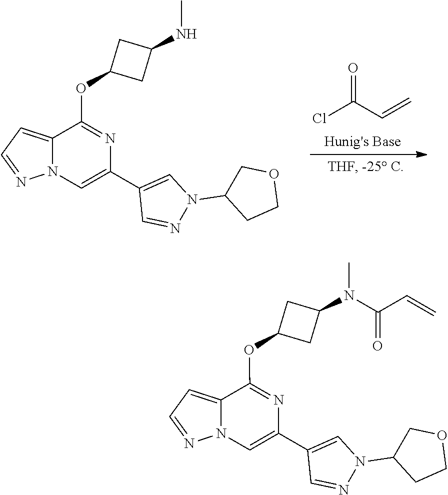

To a vial containing cis N-methyl-3-((6-(1-(tetrahydrofuran-3-yl)-1H-pyrazol-4-yl)pyrazolo[1,5-a]pyrazin-4-yl)oxy)cyclobutan-1-amine trifluoroacetate (308 mg, 869 μmol) in anhydrous THF (3 mL) was added Hunig's base (1.6 mL, 9.2 mmol) dropwise at −25° C. Acryloyl chloride (0.08 mL, 985 μmol) was added dropwise and the reaction stirred for 3 mins, then carefully quenched with 1 M NaOH solution. The mixture was stirred at 23° C. for 1 h and was then extracted with EtOAc (3×). The combined organic phases were washed with saturated aqueous NaHCO₃ solution. The organic layer was dried over anhydrous Na₂SO₄, filtered and concentrated under reduced pressure. The residue was purified by silica gel column (20-70% 3:1 EtOAc:EtOH in heptane) to afford cis N-methyl-N-(3-((6-(1-(tetrahydrofuran-3-yl)-1H-pyrazol-4-yl)pyrazolo[1,5-a]pyrazin-4-yl)oxy)cyclobutyl)acrylamide as a sticky white foam (207 mg, yield: 53%). ESI-MS (M+H)⁺: 409.2.

### 5. Synthesis of cis (R)—N-methyl-N-(3-((6-(1-(tetrahydrofuran-3-yl)-1H-pyrazol-4-yl)pyrazolo[1,5-a]pyrazin-4-yl)oxy)cyclobutyl)acrylamide and cis (S)—N-methyl-N-(3-((6-(1-(tetrahydrofuran-3-yl)-1H-pyrazol-4-yl)pyrazolo[1,5-a]pyrazin-4-yl)oxy)cyclobutyl)acrylamide

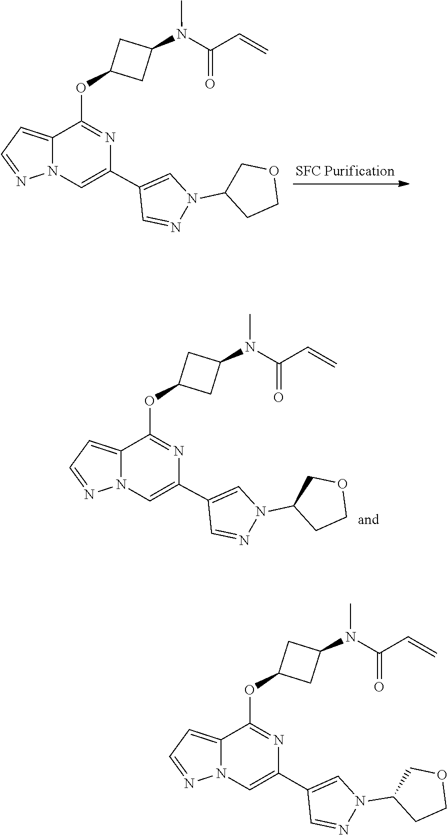 [SMILES: C=CC(=O)N(C)[C@H]1C[C@@H](Oc2nc(-c3cnn(C4CCOC4)c3)cn3nccc23)C1 ; C=CC(=O)N(C)[C@H]1C[C@@H](Oc2nc(-c3cnn([C@@H]4CCOC4)c3)cn3nccc23)C1 ; C=CC(=O)N(C)[C@H]1C[C@@H](Oc2nc(-c3cnn([C@H]4CCOC4)c3)cn3nccc23)C1]

cis N-Methyl-N-(3-((6-(1-(tetrahydrofuran-3-yl)-1H-pyrazol-4-yl)pyrazolo[1,5-a]pyrazin-4-yl)oxy)cyclobutyl)acrylamide (207 mg, 507 μmol) was resolved by chiral SFC purification (LUX Cellulose-4 LC, 5 mm column using 40% MeOH in CO₂. Flow rate: 100 mL/min; ABPR 120 bar; MBPR 40 psi, column temperature 40° C.) to afford the following compounds that were concentrated to dryness then lyophilized:

### Example 155: to provide the first eluting peak, as a brown solid (62 mg yield: 28%). ESI-MS (M+H)⁺: 409.1. ¹H NMR (500 MHz, DMSO-d₆) δ=8.81 (s, 1H), 8.32 (s, 1H), 8.08 (s, 1H), 8.03 (d, J=1.8 Hz, 1H), 6.89-6.85 (m, 1H), 6.85-6.69 (m, 1H), 6.11 (br d, J=16.5 Hz, 1H), 5.70 (dd, J=2.4, 10.4 Hz, 1H), 5.22-5.11 (m, 1H), 5.11-5.07 (m, 1H), 4.80-4.39 (m, 1H), 4.05-3.99 (m, 2H), 3.95-3.91 (m, 1H), 3.89-3.82 (m, 1H), 3.08-2.83 (m, 5H), 2.48-2.29 (m, 4H)

### Example 156: the second eluting peak as a brown solid (64 mg, yield: 28%). ESI-MS (M+H)⁺: 409.2. ¹H NMR (500 MHz, DMSO-d₆) δ=8.80 (s, 1H), 8.31 (s, 1H), 8.07 (s, 1H), 8.03 (d, J=1.8 Hz, 1H), 6.86 (d, J=1.8 Hz, 1H), 6.84-6.69 (m, 1H), 6.11 (br d, J=15.3 Hz, 1H), 5.69 (dd, J=2.4, 10.4 Hz, 1H), 5.21-5.11 (m, 1H), 5.10-5.05 (m, 1H), 4.79-4.40 (m, 1H), 4.04-3.99 (m, 2H), 3.95-3.91 (m, 1H), 3.87-3.82 (m, 1H), 3.05-2.83 (m, 5H), 2.47-2.29 (m, 4H)

### Example 157: Cis N-(3-methyl-3-((3-methyl-6-(1-(trifluoromethyl)-1H-pyrazol-4-yl)pyrazolo[1,5-a]pyrazin-4-yl)oxy)cyclobutyl)but-2-ynamide

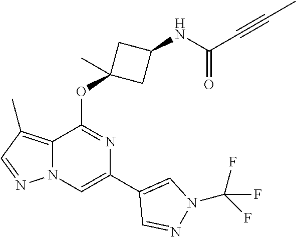 [SMILES: CC#CC(=O)N[C@H]1C[C@@](C)(Oc2nc(-c3cnn(C(F)(F)F)c3)cn3ncc(C)c23)C1]

### 1. Synthesis of 4,6-dichloro-3-iodo-pyrazolo[1,5-a]pyrazine

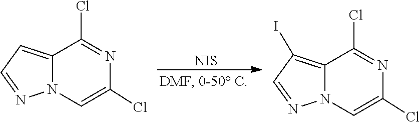 [SMILES: Clc1cn2nccc2c(Cl)n1 ; "NIS" ; Clc1cn2ncc(I)c2c(Cl)n1]

NIS (13.4 g, 59.6 mmol) was added carefully in portions to an ice-cooled solution of 4,6-dichloropyrazolo[1,5-a]pyrazine (4.97 g, 26.4 mmol) in anhydrous DMF (30 mL) and after 15 mins, the reaction was heated to 50° C. and stirred at 20 h. The cooled mixture was diluted with 1 M aqueous sodium thiosulfate solution, then extracted with EtOAc (3×). The combined organic layers were washed with saturated aqueous NaHCO₃ solution, dried over anhydrous Na₂SO₄, filtered and concentrated in vacuo. The residue was purified by silica gel column (5-20% EtOAc in heptane) to give 4,6-dichloro-3-iodo-pyrazolo[1,5-a]pyrazine as a white solid (4.6 g, yield: 55%). ¹H NMR (500 MHz, DMSO-d₆) δ=9.35 (s, 1H), 8.40 (s, 1H).

### 2. Synthesis of tert-butyl ((cis)-3-((6-chloro-3-methylpyrazolo[1,5-a]pyrazin-4-yl)oxy)-3-methylcyclobutyl)carbamate

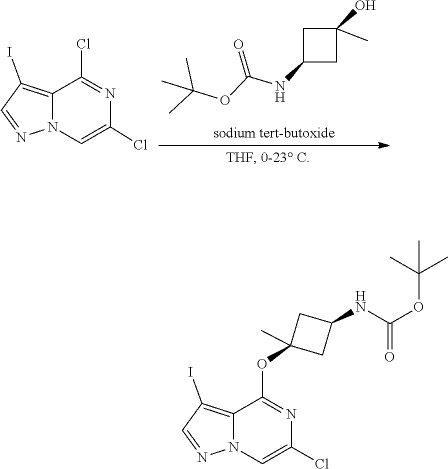

NaOtBu (738 mg, 7.68 mmol) was added in portions to an ice-cooled mixture of tert-butyl ((cis)-3-hydroxy-3-methylcyclobutyl)carbamate (970 mg, 4.82 mmol) in anhydrous THF (14 mL). After 15 mins, 4,6-dichloro-3-iodo-pyrazolo[1,5-a]pyrazine (1.38 g, 4.39 mmol) was added in portions and the reaction then stirred at 23° C. for 45 mins. The reaction was quenched by the slow addition of water and the biphasic mixture was extracted with EtOAc (3×). The combined organics were dried over anhydrous Na₂SO₄, filtered and concentrated in vacuo. The residue was purified by silica gel column (5-30% EtOAc in heptane) to give tert-butyl ((cis)-3-((6-chloro-3-iodopyrazolo[1,5-a]pyrazin-4-yl)oxy)-3-methylcyclobutyl)carbamate as an off-white solid (1.99 g, yield: 95%) ESI-MS (M+H)⁺: 478.9.

### 3. Synthesis of cis tert-butyl N-(3-(6-chloro-3-methyl-pyrazolo[1,5-a]pyrazin-4-yl)oxy-3-methyl-cyclobutyl)carbamate

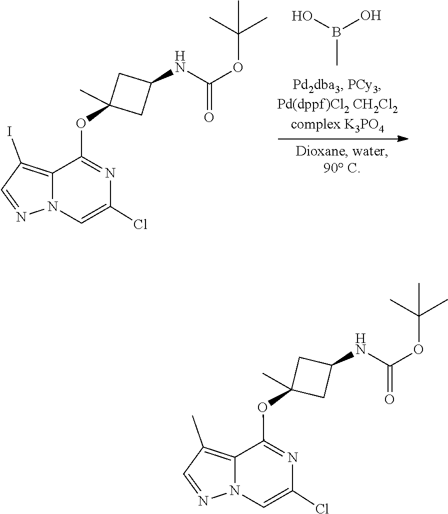

A flask containing ((cis)-3-((6-chloro-3-iodopyrazolo[1,5-a]pyrazin-4-yl)oxy)-3-methylcyclobutyl)carbamate (1.99 g, 4.16 mmol), methylboronic acid (556 mg, 9.28 mmol), tricyclohexylphosphine (311 mg, 1.11 mmol), Pd₂dba₃ (408 mg, 0.445 mmol), Pd(dppf)Cl₂:DCM (405 mg, 0.496 mmol) and K₃PO₄, 1.0 M solution (12.8 mL) in dioxane (15 mL) was degassed and purged with N₂. The reaction mixture was heated to 90° C. and stirred for 80 mins, then allowed to cool to RT. The reaction was partitioned between water and EtOAc, the layers separated and the aquoeus layer was extracted with EtOAc (2×). The combined organic extracts were washed with brine then dried over Na₂SO₄, filtered and concentrated in vacuo. The residue was purified by silica gel column (5-35% EtOAc in heptane) and the product re-purified by silica gel column (10% EtOAc in heptane) to give cis tert-butyl N-(3-(6-chloro-3-methyl-pyrazolo[1,5-a]pyrazin-4-yl)oxy-3-methyl-cyclobutyl)carbamate as an off-white solid (292 mg, yield: 19%). ESI-MS (M+H)⁺: 367.0.

### 4. Synthesis of tert-butyl ((cis)-3-methyl-3-((3-methyl-6-(1-(trifluoromethyl)-1H-pyrazol-4-yl)pyrazolo[1,5-a]pyrazin-4-yl)oxy)cyclobutyl)carbamate

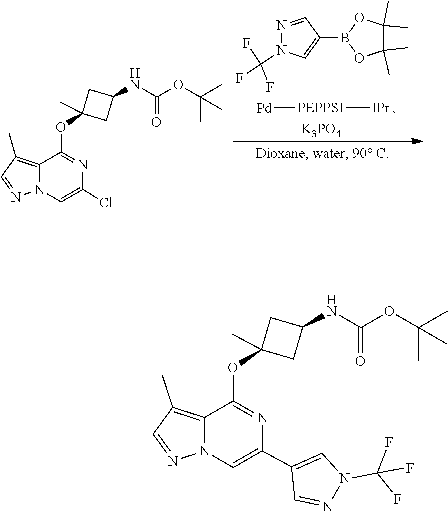

A vial containing tert-butyl ((cis)-3-((6-chloro-3-methylpyrazolo[1,5-a]pyrazin-4-yl)oxy)-3-methylcyclobutyl)carbamate (292 mg, 795 μmol), 4-(4,4,5,5-tetramethyl-1,3,2-dioxaborolan-2-yl)-1-(trifluoromethyl)pyrazole (398 mg, 1.52 mmol), K₃PO₄, 1M solution (2.4 mL), and Pd-PEPPSI™-IPr (117 mg, 171 μmol), in dioxane (4 mL) was degassed then purged with N₂. The reaction was heated to 90° C. and stirred for 45 mins. The reaction was quenched with slow addition of water, the biphasic mixture was extracted with EtOAc (3×) and the combined organic extracts were dried over anhydrous Na₂SO₄. The mixture was filtered, concentrated in vacuo and the residue was purified by silica gel column (10-40% EtOAc in heptane) to give tert-butyl ((cis)-3-methyl-3-((3-methyl-6-(1-(trifluoromethyl)-1H-pyrazol-4-yl)pyrazolo[1,5-a]pyrazin-4-yl)oxy)cyclobutyl)carbamate as a white solid (354 mg, yield: 95%) ESI-MS (M+H)⁺: 467.0.

### 5. Synthesis of cis-3-methyl-3-((3-methyl-6-(1-(trifluoromethyl)-1H-pyrazol-4-yl)pyrazolo[1,5-a]pyrazin-4-yl)oxy)cyclobutan-1-amine

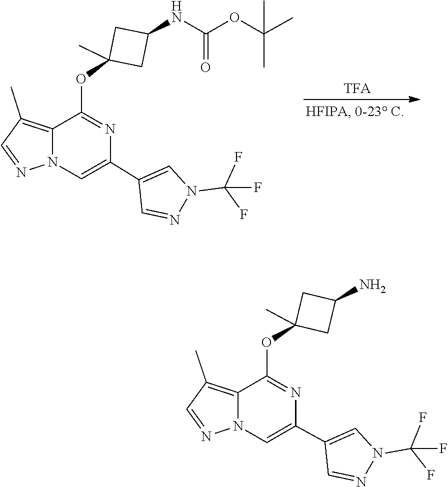 [SMILES: Cc1cnn2cc(-c3cnn(C(F)(F)F)c3)nc(O[C@]3(C)C[C@H](NC(=O)OC(C)(C)C)C3)c12 ; Cc1cnn2cc(-c3cnn(C(F)(F)F)c3)nc(O[C@]3(C)C[C@H](N)C3)c12]

TFA (0.1 mL, 1.31 mmol) was added dropwise to a solution of tert-butyl ((cis)-3-methyl-3-((3-methyl-6-(1-(trifluoromethyl)-1H-pyrazol-4-yl)pyrazolo[1,5-a]pyrazin-4-yl)oxy)cyclobutyl)carbamate (191 mg, 409 μmol) in HFIPA (2.1 mL) cooled in an ice water bath and the reaction then allowed to warm to RT and stirred for 3.5 h. The reaction was evaporated under reduced pressure to afford cis-3-methyl-3-((3-methyl-6-(1-(trifluoromethyl)-1H-pyrazol-4-yl)pyrazolo[1,5-a]pyrazin-4-yl)oxy)cyclobutan-1-amine trifluoroacetate as a colorless film (200 mg) that was used without purification. ESI-MS (M+H)⁺: 367.1.

### 6. Synthesis of N-cis-3-methyl-3-((3-methyl-6-(1-(trifluoromethyl)-1H-pyrazol-4-yl)pyrazolo[1,5-a]pyrazin-4-yl)oxy)cyclobutyl)but-2-ynamide

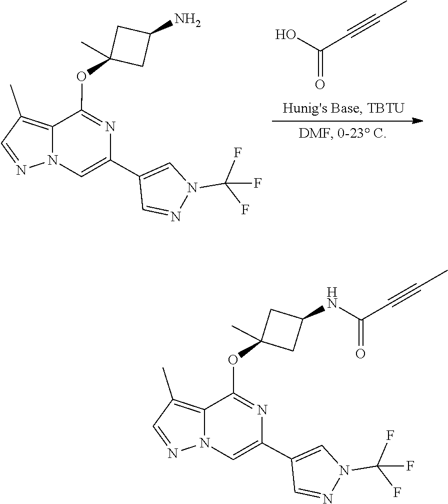

Hunig's Base (0.4 mL, 2.3 mmol) and TBTU (160 mg, 0.5 mmol) were added carefully to a solution of cis-3-methyl-3-((3-methyl-6-(1-(trifluoromethyl)-1H-pyrazol-4-yl)pyrazolo[1,5-a]pyrazin-4-yl)oxy)cyclobutan-1-amine trifluoroacetate (100 mg, 273 μmol) and but-2-ynoic acid (49 mg, 578 μmol) in anhydrous DMF (1.5 mL) cooled in an ice bath and the reaction allowed to warm to RT and stirred for 3 h. The reaction was diluted with saturated aqueous NaHCO₃ solution, then extracted with EtOAc (3×). The combined organic layers were washed with brine, dried over anhydrous MgSO₄, filtered and concentrated under reduced pressure. The residue was purified by silica gel column (10-60% EtOAc in heptane). The product was re-purified by reverse phase HPLC (Waters XSelect CSH C18, 5 μmm, 50 mm×100 mm column with mobile phase H₂O (A) and MeCN (B) and a gradient of 5-75% B (0.2% NH₄OH final v/v % modifier) with flow rate at 80 mL/min, to give N-cis-3-methyl-3-((3-methyl-6-(1-(trifluoromethyl)-1H-pyrazol-4-yl)pyrazolo[1,5-a]pyrazin-4-yl)oxy)cyclobutyl)but-2-ynamide as a white solid (24.3 mg, yield: 20%). ESI-MS (M+H)⁺: 433.1. ¹H NMR (500 MHz, DMSO-d₆) δ=8.87 (s, 1H), 8.82-8.78 (m, 2H), 8.48 (s, 1H), 7.89 (s, 1H), 4.15-4.07 (m, 1H), 2.82 (ddd, J=2.4, 7.3, 9.8 Hz, 2H), 2.40 (s, 4H), 2.38-2.35 (m, 1H), 1.93 (s, 3H), 1.74 (s, 3H).

### Examples 158 & 159: Cis N-methyl-N-(1-methyl-3-((6-(1-methyl-1H-pyrazol-4-yl)pyrazolo[1,5-a]pyrazin-4-yl)oxy)cyclobutyl)acrylamide and trans N-methyl-N-(1-methyl-3-((6-(1-methyl-1H-pyrazol-4-yl)pyrazolo[1,5-a]pyrazin-4-yl)oxy)cyclobutyl)acrylamide

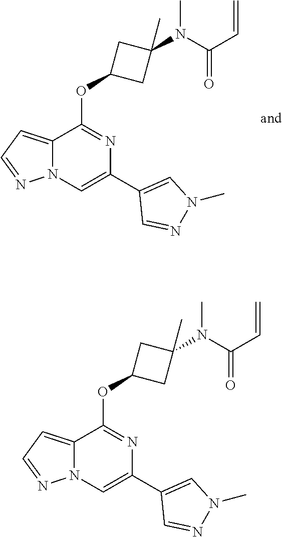 [SMILES: C=CC(=O)N(C)[C@]1(C)C[C@H](Oc2nc(-c3cnn(C)c3)cn3nccc23)C1 ; C=CC(=O)N(C)[C@]1(C)C[C@@H](Oc2nc(-c3cnn(C)c3)cn3nccc23)C1]

### 1. Synthesis of tert-butyl methyl(1-methyl-3-((6-(1-methyl-1H-pyrazol-4-yl)pyrazolo[1,5-a]pyrazin-4-yl)oxy)cyclobutyl)carbamate

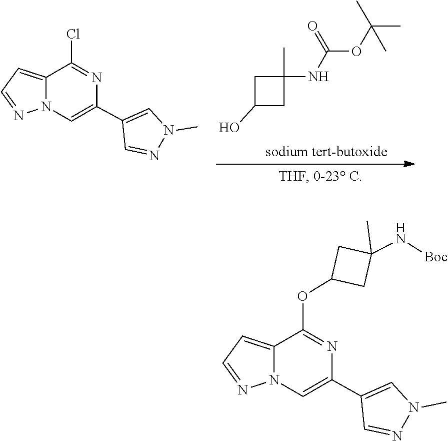

NaOtBu (311 mg, 3.24 mmol) was added in portions to an ice cooled solution of tert-butyl N-(3-hydroxy-1-methyl-cyclobutyl)carbamate (355 mg, 1.76 mmol) in anhydrous THF (3 mL) and the mixture stirred for 10 mins. 4-Chloro-6-(1-methylpyrazol-4-yl)pyrazolo[1,5-a]pyrazine (Intermediate A, 341 mg, 1.46 mmol) was added, the mixture was allowed to warm to 23° C. and stirred for 1.5 h. The reaction was quenched with saturated aqueous NaHCO₃ solution, then the biphasic mixture was extracted with EtOAc (3×). The combined organics were dried over anhydrous Na₂SO₄, filtered and concentrated under reduced pressure. The residue was purified by silica gel column (30-85% EtOAc in heptane) to give tert-butyl methyl(1-methyl-3-((6-(1-methyl-1H-pyrazol-4-yl)pyrazolo[1,5-a]pyrazin-4-yl)oxy)cyclobutyl)carbamate as a white solid (467 mg, yield: 80%). ESI-MS (M+H)⁺: 399.2.

### 2. Synthesis of tert-butyl methyl(1-methyl-3-((6-(1-methyl-1H-pyrazol-4-yl)pyrazolo[1,5-a]pyrazin-4-yl)oxy)cyclobutyl)carbamate

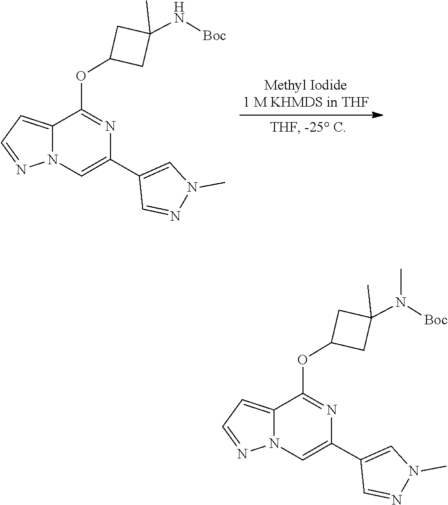 [SMILES: Cn1cc(-c2cn3nccc3c(OC3CC(C)(NC(=O)OC(C)(C)C)C3)n2)cn1 ; CN(C(=O)OC(C)(C)C)C1(C)CC(Oc2nc(-c3cnn(C)c3)cn3nccc23)C1]

KHDMS, 1M in THF (1 M, 1.8 mL) was added dropwise to a solution of tert-butyl methyl(1-methyl-3-((6-(1-methyl-1H-pyrazol-4-yl)pyrazolo[1,5-a]pyrazin-4-yl)oxy)cyclobutyl)carbamate (236 mg, 591 μmol) in anhydrous THF (4 mL) cooled to −25° C. under N₂ and the mixture stirred for 10 mins. Methyl iodide (0.08 mL, 1.29 mmol) was added dropwise and upon complete addition, the reaction was warmed to 23° C. and stirred for 2 h. The reaction was quenched with water and the biphasic mixture was extracted with EtOAc (3×). The combined organics were dried over anhydrous Na₂SO₄, filtered and concentrated under reduced pressure. The residue was purified by silica gel column (20-70% EtOAc in heptane) to afford tert-butyl methyl(1-methyl-3-((6-(1-methyl-1H-pyrazol-4-yl)pyrazolo[1,5-a]pyrazin-4-yl)oxy)cyclobutyl)carbamate as a white foam. ESI-MS (M+H)⁺: 413.2.

### 3. Synthesis of N,1-dimethyl-3-[6-(1-methylpyrazol-4-yl)pyrazolo[1,5-a]pyrazin-4-yl]oxy-cyclobutanamine trifluoroacetate

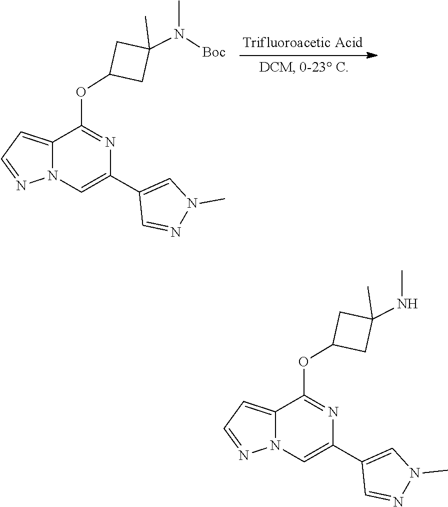 [SMILES: CN(C(=O)OC(C)(C)C)C1(C)CC(Oc2nc(-c3cnn(C)c3)cn3nccc23)C1 ; CNC1(C)CC(Oc2nc(-c3cnn(C)c3)cn3nccc23)C1]

TFA (0.35 mL, 4.6 mmol) was added to a solution of tert-butyl methyl(1-methyl-3-((6-(1-methyl-1H-pyrazol-4-yl)pyrazolo[1,5-a]pyrazin-4-yl)oxy)cyclobutyl)carbamate (215 mg, 521 μmol) in anhydrous DCM (2 mL) cooled in an ice water bath, and the reaction then stirred at 23° C. for 2 h. The reaction was evaporated under reduced pressure to afford N,1-dimethyl-3-[6-(1-methylpyrazol-4-yl)pyrazolo[1,5-a]pyrazin-4-yl]oxy-cyclobutanamine trifluoroacetate as a colorless film (221 mg) that was used without purification. ESI-MS (M+H)⁺: 313.1.

### 4. Synthesis of N-methyl-N-(1-methyl-3-((6-(1-methyl-1H-pyrazol-4-yl)pyrazolo[1,5-a]pyrazin-4-yl)oxy)cyclobutyl)acrylamide

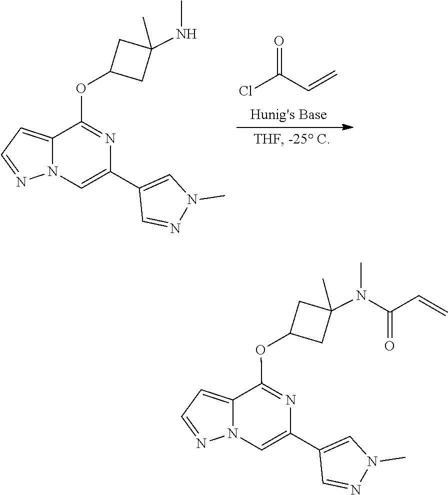

N-Methyl-N-(1-methyl-3-((6-(1-methyl-1H-pyrazol-4-yl)pyrazolo[1,5-a]pyrazin-4-yl)oxy)cyclobutyl)acrylamide was obtained as a colorless film from N,1-dimethyl-3-[6-(1-methylpyrazol-4-yl)pyrazolo[1,5-a]pyrazin-4-yl]oxy-cyclobutanamine trifluoroacetate and acryloyl chloride, following a similar procedure to that described in step 4, Example 155 (124.4 mg, yield: 48%). ESI-MS (M+H)⁺: 367.1. ¹H NMR (500 MHz, DMSO-d₆) d=8.76 (d, J=3.7 Hz, 1H), 8.22-8.15 (m, 1H), 8.06-7.97 (m, 2H), 6.91-6.80 (m, 1H), 6.73-6.35 (m, 1H), 6.09 (br t, J=14.0 Hz, 1H), 5.70-5.60 (m, 1H), 5.45-5.20 (m, 1H), 3.89 (d, J=14.0 Hz, 3H), 2.98-2.79 (m, 5H), 2.49-2.22 (m, 2H), 1.57-1.38 (m, 3H).

### 5. Synthesis of N-methyl-N-((cis)-1-methyl-3-((6-(1-methyl-1H-pyrazol-4-yl)pyrazolo[1,5-a]pyrazin-4-yl)oxy)cyclobutyl)acrylamide and N-methyl-N-((trans)-1-methyl-3-((6-(1-methyl-1H-pyrazol-4-yl)pyrazolo[1,5-a]pyrazin-4-yl)oxy)cyclobutyl)acrylamide

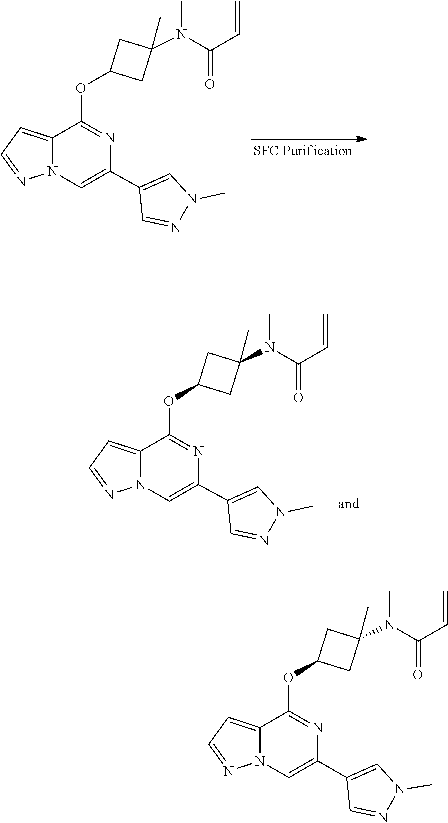 [SMILES: C=CC(=O)N(C)C1(C)CC(Oc2nc(-c3cnn(C)c3)cn3nccc23)C1 ; C=CC(=O)N(C)[C@]1(C)C[C@H](Oc2nc(-c3cnn(C)c3)cn3nccc23)C1 ; C=CC(=O)N(C)[C@]1(C)C[C@@H](Oc2nc(-c3cnn(C)c3)cn3nccc23)C1]

N-Methyl-N-(1-methyl-3-((6-(1-methyl-1H-pyrazol-4-yl)pyrazolo[1,5-a]pyrazin-4-yl)oxy)cyclobutyl)acrylamide (124 mg, 338 μmol) was purified by chiral SFC purification (Chiralpak IA 30×250 mm, 5 mm column using 30% MeOH in CO₂. Flow rate: 100 mL/min; ABPR 120 bar; MBPR 40 psi, column temperature 40° C.) to afford the following compounds that were concentrated to dryness then lyophilized:

### Example 158: To give, the first eluting peak, Rf=3.87 min as a white solid (44 mg, yield: 34%). ESI-MS (M+H)⁺: 367.1. ¹H NMR (500 MHz, DMSO-d₆) d=8.75 (s, 1H), 8.20 (s, 1H), 8.01 (d, J=2.4 Hz, 2H), 6.82 (s, 1H), 6.71-6.41 (m, 1H), 6.07 (br d, J=16.5 Hz, 1H), 5.63 (dd, J=2.4, 10.4 Hz, 1H), 5.32-5.19 (m, 1H), 3.91 (s, 3H), 2.96-2.79 (m, 5H), 2.53-2.51 (m, 1H), 2.36 (br d, J=1.8 Hz, 1H), 1.57-1.40 (m, 3H)

### Example 159: And the second eluting peak Rf=3.99 min as a colorless film (44 mg, yield: 34%). ESI-MS (M+H)⁺: 367.1. ¹H NMR (500 MHz, DMSO-d₆) d=8.77 (s, 1H), 8.17 (s, 1H), 8.04 (d, J=2.4 Hz, 1H), 7.99 (s, 1H), 6.89 (d, J=1.2 Hz, 1H), 6.69 (br s, 1H), 6.11 (br d, J=16.5 Hz, 1H), 5.65 (br d, J=10.4 Hz, 1H), 5.41 (br s, 1H), 3.93-3.83 (m, 3H), 3.05-2.82 (m, 5H), 2.38-2.24 (m, 2H), 1.50 (br s, 3H)

Examples 160 & 161: (R) and (S)-1-(4-((6-(1-methyl-1H-pyrazol-4-yl)pyrazolo[1,5-a]pyrazin-4-yl)amino)azepan-1-yl)prop-2-en-1-one

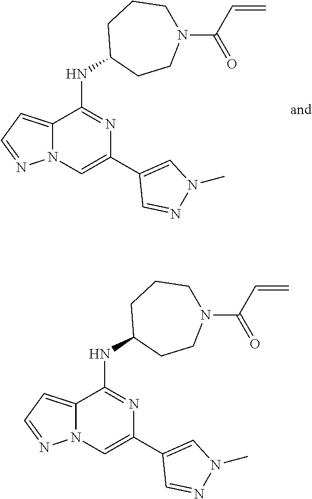 [SMILES: C=CC(=O)N1CCC[C@@H](Nc2nc(-c3cnn(C)c3)cn3nccc23)CC1 ; C=CC(=O)N1CCC[C@H](Nc2nc(-c3cnn(C)c3)cn3nccc23)CC1]

### 1. Synthesis of tert-butyl 4-((6-(1-methyl-1H-pyrazol-4-yl)pyrazolo[1,5-a]pyrazin-4-yl)amino)azepane-1-carboxylate

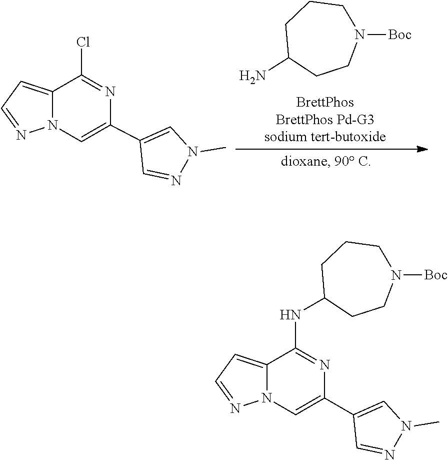 [SMILES: Cn1cc(-c2cn3nccc3c(Cl)n2)cn1 ; CC(C)(C)OC(=O)N1CCCC(N)CC1 ; Cn1cc(-c2cn3nccc3c(NC3CCCN(C(=O)OC(C)(C)C)CC3)n2)cn1]

A mixture of tert-butyl 4-aminoazepane-1-carboxylate (407 mg, 1.9 mmol), 4-chloro-6-(1-methylpyrazol-4-yl)pyrazolo[1,5-a]pyrazine (Intermediate A, 236 mg, 1.01 mmol), BrettPhos (79 mg, 148 μmol), BrettPhos Pd G3 (132 mg, 145 μmol) and NaOtBu (327 mg, 3.4 mmol) in dioxane (2 mL) was degassed and backfilled with N₂ (3×). The reaction mixture was heated to 90° C. and stirred for 2 h. The reaction was quenched with water, then the biphasic mixture was extracted with EtOAc (3×). The combined organics were dried over anhydrous Na₂SO₄, filtered and concentrated under reduced pressure. The residue was purified by silica gel column (30-100% EtOAc in heptane) to give tert-butyl 4-((6-(1-methyl-1H-pyrazol-4-yl)pyrazolo[1,5-a]pyrazin-4-yl)amino)azepane-1-carboxylate as a white solid (408 mg, yield: 98% yield). ESI-MS (M+H)⁺: 412.2.

### 2. Synthesis of N-(azepan-4-yl)-6-(1-methyl-1H-pyrazol-4-yl)pyrazolo[1,5-a]pyrazin-4-amine trifluoroacetate

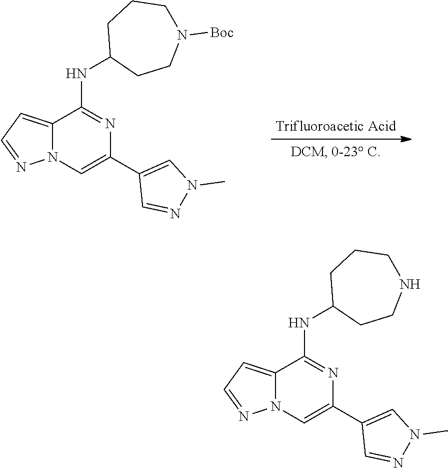 [SMILES: Cn1cc(-c2cn3nccc3c(NC3CCCN(C(=O)OC(C)(C)C)CC3)n2)cn1 ; Cn1cc(-c2cn3nccc3c(NC3CCCNCC3)n2)cn1]

N-(Azepan-4-yl)-6-(1-methyl-1H-pyrazol-4-yl)pyrazolo[1,5-a]pyrazin-4-amine trifluoroacetate was obtained as a yellow film, from tert-butyl 4-((6-(1-methyl-1H-pyrazol-4-yl)pyrazolo[1,5-a]pyrazin-4-yl)amino)azepane-1-carboxylate, following the procedure described in Example 158 (177 mg, quantitative yield). ESI-MS (M+H)⁺: 312.1.

### 3. Synthesis of 1-(4-((6-(1-methyl-1H-pyrazol-4-yl)pyrazolo[1,5-a]pyrazin-4-yl)amino)azepan-1-yl)prop-2-en-1-one

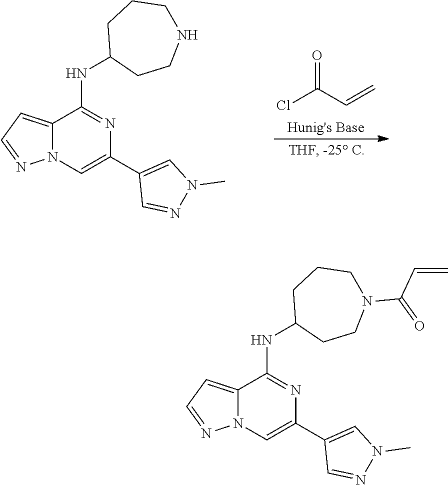

1-(4-((6-(1-Methyl-1H-pyrazol-4-yl)pyrazolo[1,5-a]pyrazin-4-yl)amino)azepan-1-yl)prop-2-en-1-one was obtained as a yellow solid, 100 mg, 44% yield, from N-(azepan-4-yl)-6-(1-methyl-1H-pyrazol-4-yl)pyrazolo[1,5-a]pyrazin-4-amine trifluoroacetate and acryloyl chloride, following a similar procedure to that described in step 3, Example 142. ESI-MS (M+H)⁺: 366.1.

### 4. Synthesis of (R) or (S)-1-(4-((6-(1-methyl-1H-pyrazol-4-yl)pyrazolo[1,5-a]pyrazin-4-yl)amino)azepan-1-yl)prop-2-en-1-one

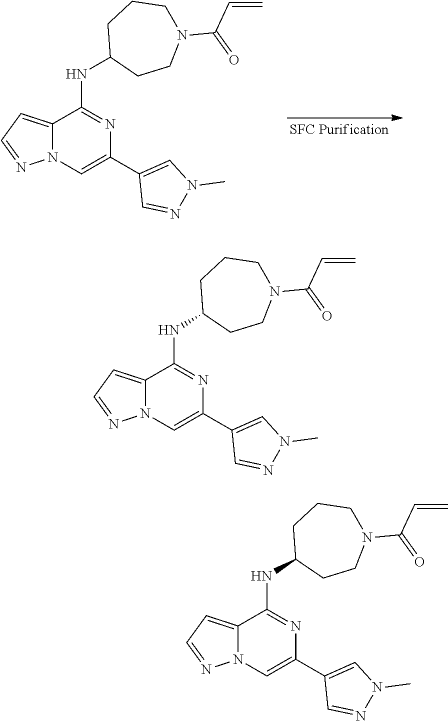 [SMILES: C=CC(=O)N1CCCC(Nc2nc(-c3cnn(C)c3)cn3nccc23)CC1 ; C=CC(=O)N1CCC[C@@H](Nc2nc(-c3cnn(C)c3)cn3nccc23)CC1 ; C=CC(=O)N1CCC[C@H](Nc2nc(-c3cnn(C)c3)cn3nccc23)CC1]

1-(4-((6-(1-Methyl-1H-pyrazol-4-yl)pyrazolo[1,5-a]pyrazin-4-yl)amino)azepan-1-yl)prop-2-en-1-one (70 mg, 190 μmol) was purified by SFC (Chiralpak IG 30×250 mm, column using 45% IPA in CO₂. Flow rate: 100 mL/min; ABPR 120 bar; MBPR 60 psi, column temperature 40° C. to afford the following compounds that were concentrated to dryness:

### Example 160: To give Peak 1, Rf=6.97 min as a colorless film (31.7 mg, yield: 43%). ESI-MS (M+H)⁺: 366.2. ¹H NMR (500 MHz, DMSO-d₆) d=8.27 (s, 1H), 8.13-8.04 (m, 1H), 7.91 (d, J=3.7 Hz, 1H), 7.83 (t, J=2.1 Hz, 1H), 7.43 (d, J=7.3 Hz, 1H), 6.96 (d, J=1.8 Hz, 1H), 6.83 (ddd, J=7.6, 10.4, 16.8 Hz, 1H), 6.24-6.14 (m, 1H), 5.70 (ddd, J=3.1, 4.7, 10.5 Hz, 1H), 4.19 (br s, 1H), 3.87 (s, 3H), 3.81-3.51 (m, 4H), 2.26-2.13 (m, 1H), 1.98 (br s, 1H), 1.95-1.88 (m, 1H), 1.84-1.68 (m, 2H), 1.66-1.57 (m, 1H)

### Example 161: And Peak 2, Rf=8.35 min as a colorless film (32.2 mg, yield: 44% yield). ESI-MS (M+H)⁺: 366.2. ¹H NMR (500 MHz, DMSO-d₆) d=8.27 (s, 1H), 8.12-8.04 (m, 1H), 7.91 (d, J=3.7 Hz, 1H), 7.83 (t, J=1.8 Hz, 1H), 7.43 (d, J=7.3 Hz, 1H), 6.99-6.94 (m, 1H), 6.83 (ddd, J=7.6, 10.4, 16.8 Hz, 1H), 6.24-6.13 (m, 1H), 5.74-5.66 (m, 1H), 4.19 (br s, 1H), 3.87 (s, 3H), 3.81-3.47 (m, 4H), 2.26-2.12 (m, 1H), 2.03-1.96 (m, 1H), 1.95-1.88 (m, 1H), 1.83-1.69 (m, 2H), 1.66-1.56 (m, 1H)

### Examples 162 & 163: N-((1S,3R)-3-((6-(1-((R)-tetrahydrofuran-3-yl)-1H-pyrazol-4-yl)pyrazolo[1,5-a]pyrazin-4-yl)oxy)cyclohexyl)but-2-ynamide and N-((1S,3R)-3-((6-(1-((S)-tetrahydrofuran-3-yl)-1H-pyrazol-4-yl)pyrazolo[1,5-a]pyrazin-4-yl)oxy)cyclohexyl)but-2-ynamide

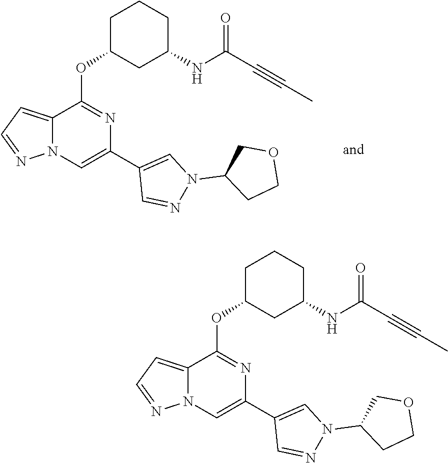 [SMILES: CC#CC(=O)N[C@H]1CCC[C@@H](Oc2nc(-c3cnn([C@@H]4CCOC4)c3)cn3nccc23)C1 ; CC#CC(=O)N[C@H]1CCC[C@@H](Oc2nc(-c3cnn([C@H]4CCOC4)c3)cn3nccc23)C1]

### 1. Synthesis of tert-butyl ((1S,3R)-3-((6-chloropyrazolo[1,5-a]pyrazin-4-yl)oxy)cyclohexyl)carbamate

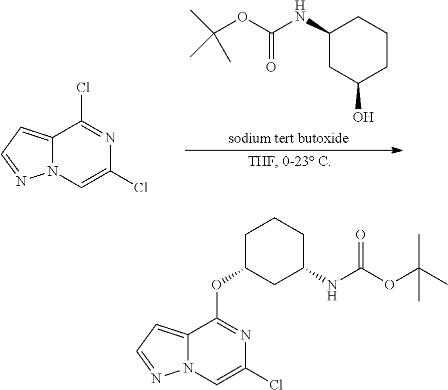 [SMILES: Clc1cn2nccc2c(Cl)n1 ; CC(C)(C)OC(=O)N[C@H]1CCC[C@@H](O)C1 ; CC(C)(C)OC(=O)N[C@H]1CCC[C@@H](Oc2nc(Cl)cn3nccc23)C1]

A vial containing tert-butyl N-((1S,3R)-3-hydroxycyclohexyl)carbamate (239 mg, 1.11 mmol) in anhydrous THF (4 mL) was cooled in an ice water bath, then NaOtBu (155 mg, 1.62 mmol) was added in portions. After 10 mins, 4,6-dichloropyrazolo[1,5-a]pyrazine (191 mg, 1.02 mmol) was added in portions and the reaction was allowed to warm to 23° C. and stirred for 3 h. The reaction was quenched with water, then the biphasic mixture was extracted with EtOAc (3×). The combined organics were dried over anhydrous Na₂SO₄, filtered and concentrated under reduced pressure. The residue was purified on silica gel column (5-30% EtOAc in heptane) to afford tert-butyl ((1S,3R)-3-((6-chloropyrazolo[1,5-a]pyrazin-4-yl)oxy)cyclohexyl)carbamate as a waxy white solid (360 mg, yield: 96%). ESI-MS (M+H)⁺: 367.1.

### 2. Synthesis of tert-butyl ((1S,3R)-3-((6-(1-(tetrahydrofuran-3-yl)-1H-pyrazol-4-yl)pyrazolo[1,5-a]pyrazin-4-yl)oxy)cyclohexyl)carbamate

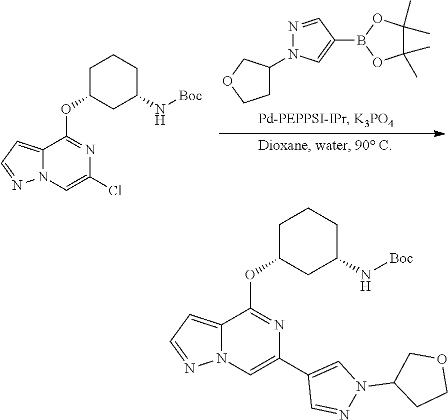 [SMILES: CC(C)(C)OC(=O)N[C@H]1CCC[C@@H](Oc2nc(Cl)cn3nccc23)C1 ; CC1(C)OB(c2cnn(C3CCOC3)c2)OC1(C)C ; CC(C)(C)OC(=O)N[C@H]1CCC[C@@H](Oc2nc(-c3cnn(C4CCOC4)c3)cn3nccc23)C1]

tert-Butyl ((1S,3R)-3-((6-(1-(tetrahydrofuran-3-yl)-1H-pyrazol-4-yl)pyrazolo[1,5-a]pyrazin-4-yl)oxy)cyclohexyl)carbamate was obtained as a colorless film, from 1-tetrahydrofuran-3-yl-4-(4,4,5,5-tetramethyl-1,3,2-dioxaborolan-2-yl)pyrazole and tert-butyl ((1S,3R)-3-((6-chloropyrazolo[1,5-a]pyrazin-4-yl)oxy)cyclohexyl)carbamate, following a similar procedure to that described in step 4, Example 157 (444 mg, yield: 97%). ESI-MS (M+H)⁺: 469.2. ¹H NMR (500 MHz, DMSO-d₆) δ=8.76 (s, 1H), 8.33 (d, J=6.7 Hz, 1H), 8.07 (s, 1H), 8.00 (d, J=2.4 Hz, 1H), 6.90 (br d, J=7.9 Hz, 1H), 6.80 (d, J=1.8 Hz, 1H), 5.28 (br t, J=10.7 Hz, 1H), 5.08 (tt, J=3.7, 6.9 Hz, 1H), 4.06-3.98 (m, 2H), 3.96-3.91 (m, 1H), 3.89-3.83 (m, 1H), 3.53-3.44 (m, 1H), 2.46-2.31 (m, 3H), 2.18-2.10 (m, 1H), 1.86-1.77 (m, 2H), 1.52-1.35 (m, 12H), 1.22-1.13 (m, 1H).

### 3. Synthesis of (1S,3R)-3-((6-(1-(tetrahydrofuran-3-yl)-1H-pyrazol-4-yl)pyrazolo[1,5-a]pyrazin-4-yl)oxy)cyclohexan-1-amine trifluoroacetate

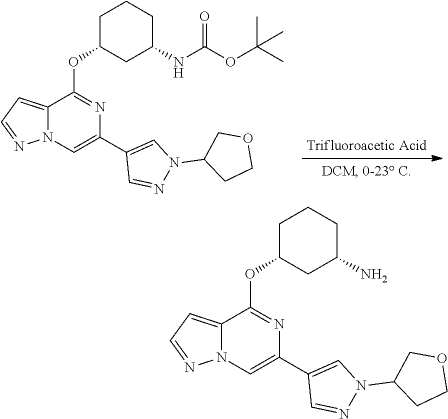 [SMILES: CC(C)(C)OC(=O)N[C@H]1CCC[C@@H](Oc2nc(-c3cnn(C4CCOC4)c3)cn3nccc23)C1 ; N[C@H]1CCC[C@@H](Oc2nc(-c3cnn(C4CCOC4)c3)cn3nccc23)C1]

(1S,3R)-3-((6-(1-(Tetrahydrofuran-3-yl)-1H-pyrazol-4-yl)pyrazolo[1,5-a]pyrazin-4-yl)oxy)cyclohexan-1-amine trifluoroacetate was obtained as a colorless film from tert-butyl ((1S,3R)-3-((6-(1-(tetrahydrofuran-3-yl)-1H-pyrazol-4-yl)pyrazolo[1,5-a]pyrazin-4-yl)oxy)cyclohexyl)carbamate, following the procedure described in step 3, Example 158 (209 mg, yield: quantitative). ESI-MS (M+H)⁺: 369.4.

### 4. Synthesis of N-((1S,3R)-3-((6-(1-(tetrahydrofuran-3-yl)-1H-pyrazol-4-yl)pyrazolo[1,5-a]pyrazin-4-yl)oxy)cyclohexyl)but-2-ynamide

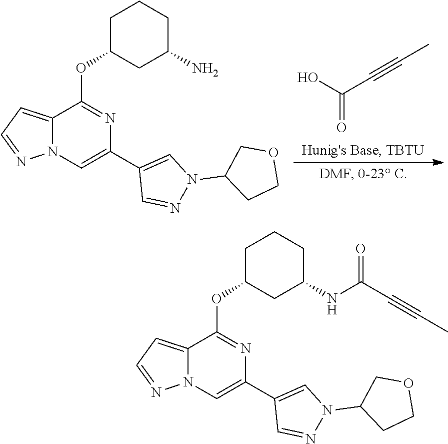

A solution of (1S,3R)-3-((6-(1-(tetrahydrofuran-3-yl)-1H-pyrazol-4-yl)pyrazolo[1,5-a]pyrazin-4-yl)oxy)cyclohexan-1-amine trifluoroacetate (100 mg, 271 μmol) and but-2-ynoic acid (46 mg, 550 μmol) in anhydrous DMF (2 mL) was cooled in an ice bath then Hunig's Base (0.59 mL, 3.39 mmol) and TBTU (156 mg, 486 μmol) were added. The reaction was warmed to 23° C. and stirred for 18 h then diluted with saturated aqueous NaHCO₃ solution. The mixture was extracted with DCM (3×), the combined organic extracts washed with brine and dried over MgSO₄, filtered and concentrated under reduced pressure. The residue was purified by silica gel column (25-70% 3:1 EtOAc:EtOH in heptane) to give N-((1S,3R)-3-((6-(1-(tetrahydrofuran-3-yl)-1H-pyrazol-4-yl)pyrazolo[1,5-a]pyrazin-4-yl)oxy)cyclohexyl)but-2-ynamide as a dark yellow solid (72 mg, yield: 61%). ESI-MS (M+H)⁺: 435.1.

### 5. Synthesis of N-((1S,3R)-3-((6-(1-((R)-tetrahydrofuran-3-yl)-1H-pyrazol-4-yl)pyrazolo[1,5-a]pyrazin-4-yl)oxy)cyclohexyl)but-2-ynamide and N-((1S,3R)-3-((6-(1-((S)-tetrahydrofuran-3-yl)-1H-pyrazol-4-yl)pyrazolo[1,5-a]pyrazin-4-yl)oxy)cyclohexyl)but-2-ynamide

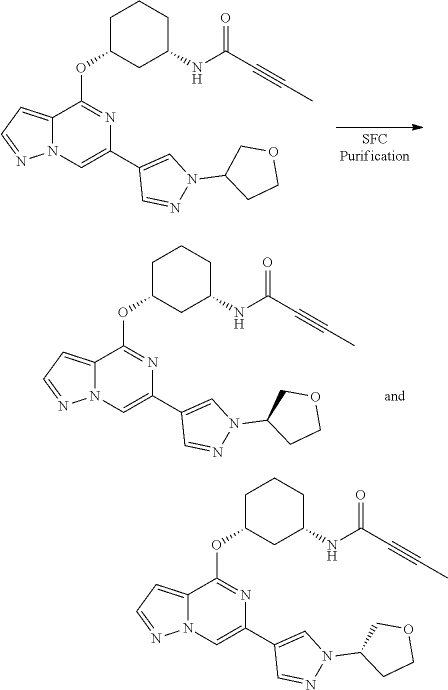

N-((1S,3R)-3-((6-(1-(Tetrahydrofuran-3-yl)-1H-pyrazol-4-yl)pyrazolo[1,5-a]pyrazin-4-yl)oxy)cyclohexyl)but-2-ynamide (70 mg, 161 μmol) was purified by SFC on a Chiralpak IG 30×250 mm, 5 mm column using 50% MeOH in CO₂. Flow rate: 100 mL/min; ABPR 120 bar; MBPR 40 psi, column temperature 40° C. to afford the following compounds that were concentrated to dryness then lyophilized:

### Example 162: To give, the first eluting Peak, Rf=4.98 min as a white solid (22 mg, yield: 30%). ESI-MS (M+H)⁺: 435.1. ¹H NMR (500 MHz, DMSO-d₆) δ=8.76 (s, 1H), 8.55 (d, J=7.3 Hz, 1H), 8.32 (s, 1H), 8.07 (s, 1H), 8.01 (d, J=1.8 Hz, 1H), 6.81 (d, J=1.8 Hz, 1H), 5.36-5.28 (m, 1H), 5.09-5.04 (m, 1H), 4.03-3.98 (m, 2H), 3.95-3.92 (m, 1H), 3.87-3.80 (m, 2H), 2.40-2.28 (m, 3H), 2.18-2.12 (m, 1H), 1.94 (s, 3H), 1.85-1.77 (m, 2H), 1.54-1.38 (m, 3H), 1.25-1.17 (m, 1H)

### Example 163: And the second eluting peak, Rf=5.51 min as a white solid (22 mg, yield: 30%). ESI-MS (M+H)⁺: 435.1. ¹H NMR (500 MHz, DMSO-d₆) δ=8.76 (s, 1H), 8.55 (br d, J=7.3 Hz, 1H), 8.31 (s, 1H), 8.07 (s, 1H), 8.01 (d, J=2.4 Hz, 1H), 6.81 (br d, J=1.8 Hz, 1H), 5.33-5.30 (m, 1H), 5.08-5.05 (m, 1H), 4.01 (br dd, J=6.1, 9.8 Hz, 2H), 3.93-3.90 (m, 1H), 3.88-3.82 (m, 2H), 2.43-2.38 (m, 2H), 2.37-2.34 (m, 1H), 2.14 (br d, J=9.8 Hz, 1H), 1.94 (s, 3H), 1.85-1.79 (m, 2H), 1.47-1.37 (m, 3H), 1.22 (br d, J=3.7 Hz, 1H)

### Example 164: N-[(1S,3R)-3-[6-(1-methylpyrazol-4-yl)pyrazolo[1,5-a]pyridin-4-yl]oxycyclopentyl]but-2-ynamide

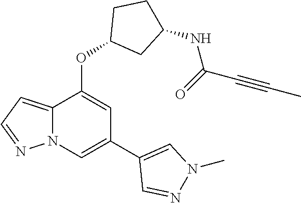 [SMILES: CC#CC(=O)N[C@H]1CC[C@@H](Oc2cc(-c3cnn(C)c3)cn3nccc23)C1]

### 1. Synthesis of tert-butyl ((1S,3R)-3-((6-(1-methyl-1H-pyrazol-4-yl)pyrazolo[1,5-a]pyridin-4-yl)oxy)cyclopentyl)carbamate

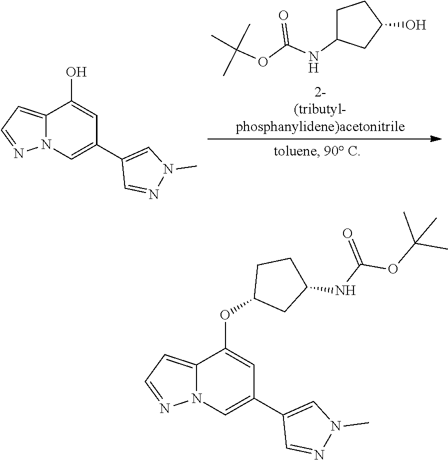 [SMILES: Cn1cc(-c2cc(O)c3ccnn3c2)cn1 ; CC(C)(C)OC(=O)NC1CC[C@H](O)C1 ; Cn1cc(-c2cc(O[C@@H]3CC[C@H](NC(=O)OC(C)(C)C)C3)c3ccnn3c2)cn1]

A vial containing 6-(1-methylpyrazol-4-yl)pyrazolo[1,5-a]pyridin-4-ol (222 mg, 1.04 mmol) and tert-butyl ((1S,3S)-3-hydroxycyclopentyl)carbamate (273 mg, 1.35 mmol) in anhydrous toluene (3.5 mL) was degassed and backfilled with N₂ then 2-(tributyl-phosphanylidene)acetonitrile (0.4 mL, 1.53 mmol) was added dropwise. The reaction was heated to 90° C. and stirred for 6 h, cooled to RT and concentrated in vacuo. The black residue was purified by silica gel column (15-90% EtOAc in heptane) to afford tert-butyl ((1S,3R)-3-((6-(1-methyl-1H-pyrazol-4-yl)pyrazolo[1,5-a]pyridin-4-yl)oxy)cyclopentyl)carbamate as a tan foam (360 mg, yield: 87%). ¹H NMR (500 MHz, DMSO-d₆) δ=8.59 (s, 1H), 8.27 (s, 1H), 8.00 (s, 1H), 7.87 (d, J=1.8 Hz, 1H), 6.94 (br d, J=7.3 Hz, 1H), 6.77 (s, 1H), 6.57 (d, J=1.2 Hz, 1H), 5.05-4.99 (m, 1H), 3.90-3.81 (m, 4H), 2.56-2.51 (m, 1H), 2.09-2.00 (m, 1H), 1.93-1.84 (m, 2H), 1.71-1.62 (m, 2H), 1.36 (s, 9H).

### 2. Synthesis of (1S,3R)-3-((6-(1-methyl-1H-pyrazol-4-yl)pyrazolo[1,5-a]pyridin-4-yl)oxy)cyclopentan-1-amine trifluoroacetate

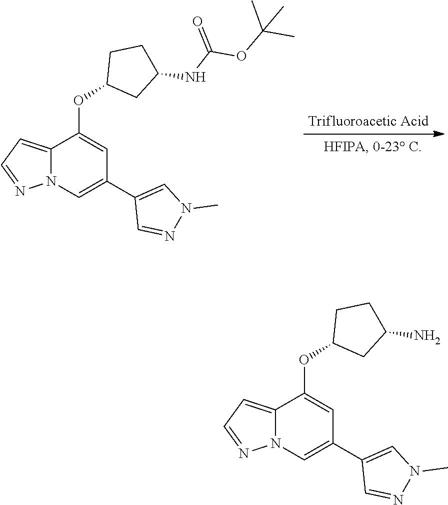 [SMILES: Cn1cc(-c2cc(O[C@@H]3CC[C@H](NC(=O)OC(C)(C)C)C3)c3ccnn3c2)cn1 ; Cn1cc(-c2cc(O[C@@H]3CC[C@H](N)C3)c3ccnn3c2)cn1]

A vial containing tert-butyl ((1S,3R)-3-((6-(1-methyl-1H-pyrazol-4-yl)pyrazolo[1,5-a]pyridin-4-yl)oxy)cyclopentyl)carbamate (172 mg, 434 μmol) in HFIPA (3 mL) was cooled in an ice water bath, TFA (0.11 mL, 1.44 mmol) was added dropwise and the reaction stirred at 23° C. for 90 mins. The reaction was concentrated under reduced pressure to afford (1S,3R)-3-((6-(1-methyl-1H-pyrazol-4-yl)pyrazolo[1,5-a]pyridin-4-yl)oxy)cyclopentan-1-amine trifluoroacetate as a brown film (178 mg, yield: 100%) that was used without purification. ESI-MS (M+H)⁺: 298.1.

### 3. Synthesis of N-((1S,3R)-3-((6-(1-methyl-1H-pyrazol-4-yl)pyrazolo[1,5-a]pyridin-4-yl)oxy)cyclopentyl)but-2-ynamide

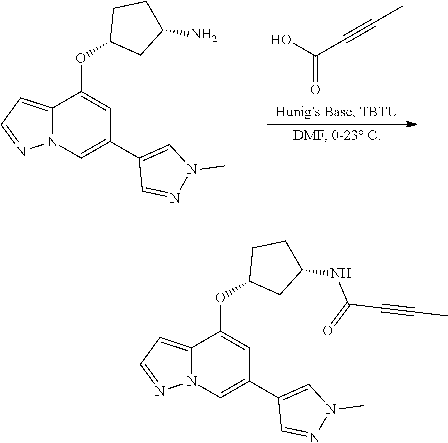

A vial containing (1S,3R)-3-((6-(1-methyl-1H-pyrazol-4-yl)pyrazolo[1,5-a]pyridin-4-yl)oxy)cyclopentan-1-amine trifluoroacetate (90 mg, 0.303 mmol) and but-2-ynoic acid (53 mg, 0.626 mmol) in anhydrous DMF (1 mL) was cooled in an ice bath, then Hunig's Base (0.43 mL, 2.47 mmol) and TBTU (251 mg, 0.780 mmol) were added and the reaction stirred for 45 mins. The heterogeneous reaction was diluted with saturated aqueous NaHCO₃ solution, the mixture was extracted with DCM (3×) and the combined organic layers were washed with brine. The organic layer was dried over anhydrous Na₂SO₄, filtered and concentrated under reduced pressure. The residue was purified by prep HPLC (Waters XSelect CSH C18, 5 μm, 50 mm×100 mm column with mobile phase H₂O (A) and MeCN (B) and a gradient of 5-75% B (0.2% NH₄OH final v/v % modifier) with flow rate at 80 mL/min to give N-((1S,3R)-3-((6-(1-methyl-1H-pyrazol-4-yl)pyrazolo[1,5-a]pyridin-4-yl)oxy)cyclopentyl)but-2-ynamide was obtained as a colorless film, 42 mg, 36% yield. ESI-MS (M+H)⁺: 364.2. ¹H NMR (500 MHz, DMSO-d₆) δ=8.63 (br d, J=6.7 Hz, 1H), 8.61-8.59 (m, 1H), 8.27 (s, 1H), 7.99 (s, 1H), 7.88 (d, J=1.8 Hz, 1H), 6.77 (s, 1H), 6.58 (d, J=1.8 Hz, 1H), 5.07-5.01 (m, 1H), 4.11-4.07 (m, 1H), 3.87 (s, 3H), 2.58-2.52 (m, 1H), 2.09-2.02 (m, 1H), 1.96-1.88 (m, 5H), 1.75-1.65 (m, 2H).

### Example 165: 1-((1R,5S,6s)-6-(((6-(1-methyl-1H-pyrazol-4-yl)pyrazolo[1,5-a]pyridin-4-yl)amino)methyl)-3-azabicyclo[3.1.0]hexan-3-yl)prop-2-en-1-one

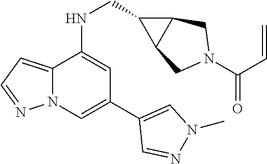 [SMILES: C=CC(=O)N1C[C@@H]2[C@@H](CNc3cc(-c4cnn(C)c4)cn4nccc34)[C@@H]2C1]

### 1. Synthesis of tert-butyl (1R,5S,6s)-6-(((6-(1-methyl-1H-pyrazol-4-yl)pyrazolo[1,5-a]pyridin-4-yl)amino)methyl)-3-azabicyclo[3.1.0]hexane-3-carboxylate

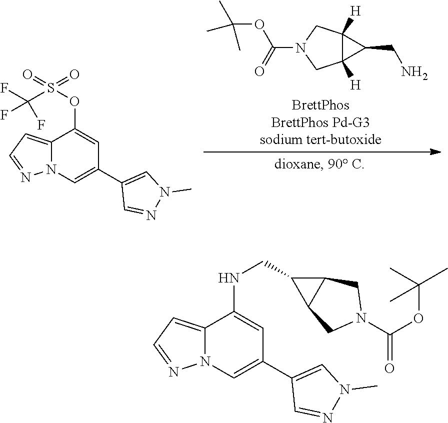 [SMILES: Cn1cc(-c2cc(OS(=O)(=O)C(F)(F)F)c3ccnn3c2)cn1 ; CC(C)(C)OC(=O)N1C[C@@H]2[C@@H](CN)[C@@H]2C1 ; Cn1cc(-c2cc(NC[C@@H]3[C@H]4CN(C(=O)OC(C)(C)C)C[C@@H]34)c3ccnn3c2)cn1]

tert-Butyl (1R,5S,6s)-6-(((6-(1-methyl-1H-pyrazol-4-yl)pyrazolo[1,5-a]pyridin-4-yl)amino)methyl)-3-azabicyclo[3.1.0]hexane-3-carboxylate was obtained as a white solid, 52 mg, 36% yield from tert-butyl (1R,5S,6s)-6-(aminomethyl)-3-azabicyclo[3.1.0]hexane-3-carboxylate and 6-(1-methyl-1H-pyrazol-4-yl)pyrazolo[1,5-a]pyridin-4-yl trifluoromethanesulfonate, following the procedure described in Example 154. ESI-MS (M+H)⁺: 409.2.

### 2. Synthesis of N-(((1R,5S,6r)-3-azabicyclo[3.1.0]hexan-6-yl)methyl)-6-(1-methyl-1H-pyrazol-4-yl)pyrazolo[1,5-a]pyridin-4-amine trifluoroacetate

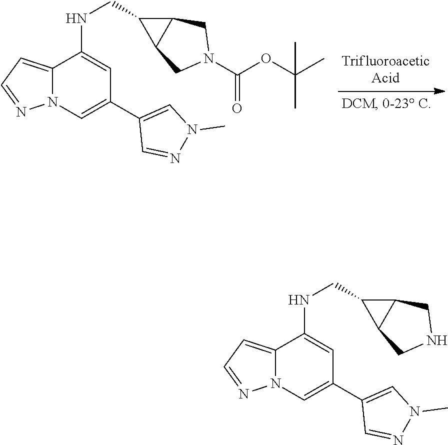 [SMILES: Cn1cc(-c2cc(NC[C@@H]3[C@H]4CN(C(=O)OC(C)(C)C)C[C@@H]34)c3ccnn3c2)cn1 ; Cn1cc(-c2cc(NC[C@H]3[C@@H]4CNC[C@@H]43)c3ccnn3c2)cn1]

N—(((1R,5S,6r)-3-azabicyclo[3.1.0]hexan-6-yl)methyl)-6-(1-methyl-1H-pyrazol-4-yl)pyrazolo[1,5-a]pyridin-4-amine trifluoroacetate was obtained as a light yellow film, 53 mg, quantitative yield, from tert-butyl (1R,5S,6s)-6-(((6-(1-methyl-1H-pyrazol-4-yl)pyrazolo[1,5-a]pyridin-4-yl)amino)methyl)-3-azabicyclo[3.1.0]hexane-3-carboxylate following the procedure described in Example 158. ESI-MS (M+H)⁺: 309.2.

### 3. Synthesis of 1-((1R,5S,6s)-6-(((6-(1-methyl-1H-pyrazol-4-yl)pyrazolo[1,5-a]pyridin-4-yl)amino)methyl)-3-azabicyclo[3.1.0]hexan-3-yl)prop-2-en-1-one

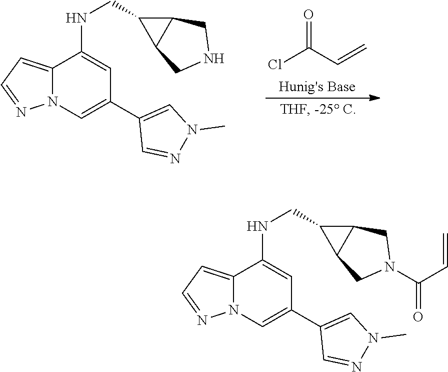

To a vial containing N—(((1R,5S,6r)-3-azabicyclo[3.1.0]hexan-6-yl)methyl)-6-(1-methyl-1H-pyrazol-4-yl)pyrazolo[1,5-a]pyridin-4-amine trifluoroacetate (53 mg, 172 μmol) in anhydrous THF (1 mL) was added Hunig's Base (0.5 mL, 2.87 mmol) dropwise at −25° C. After 5 mins, acryloyl chloride (17 mL, 209 μmol) was added dropwise and the reaction stirred for 3 mins. The reaction was quenched with 1M NaOH (aq), the mixture was stirred at 23° C. for 1 h, then the biphasic mixture was extracted with EtOAc (3×). The combined organics were washed with saturated aqueous NaHCO₃ solution, dried over anhydrous Na₂SO₄, filtered and concentrated under reduced pressure. The residue was purified by silica gel column (25-90% 3:1 EtOAc:EtOH in heptane) to afford 1-((1R,5S,6s)-6-(((6-(1-methyl-1H-pyrazol-4-yl)pyrazolo[1,5-a]pyridin-4-yl)amino)methyl)-3-azabicyclo[3.1.0]hexan-3-yl)prop-2-en-1-one as a colorless film (11 mg, yield: 19%). ESI-MS (M+H)⁺: 363.1. ¹H NMR (500 MHz, DMSO-d₆) δ=8.24 (s, 1H), 8.17 (s, 1H), 7.90 (s, 1H), 7.78 (d, J=2.4 Hz, 1H), 6.78 (d, J=1.8 Hz, 1H), 6.51 (dd, J=10.4, 16.5 Hz, 1H), 6.24 (s, 2H), 6.09 (dd, J=2.4, 17.1 Hz, 1H), 5.66-5.60 (m, 1H), 3.85 (s, 3H), 3.81-3.72 (m, 2H), 3.65-3.61 (m, 1H), 3.40-3.36 (m, 2H), 3.23-3.14 (m, 2H), 1.71-1.65 (m, 1H), 1.63-1.58 (m, 1H).

### Example 166: N-((cis)-3-((3-cyclopropyl-6-(1-methyl-1H-pyrazol-4-yl)pyrazolo[1,5-a]pyrazin-4-yl)oxy)-3-methylcyclobutyl)acrylamide

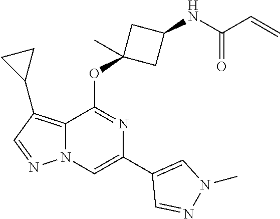 [SMILES: C=CC(=O)N[C@H]1C[C@@](C)(Oc2nc(-c3cnn(C)c3)cn3ncc(C4CC4)c23)C1]

### 1. Synthesis of 3-bromo-4,6-dichloro-pyrazolo[1,5-a]pyrazine

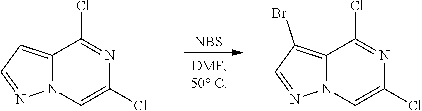 [SMILES: Clc1cn2nccc2c(Cl)n1 ; "NBS" ; "DMF,50° C." ; Clc1cn2ncc(Br)c2c(Cl)n1]

A flask containing 4,6-dichloropyrazolo[1,5-a]pyrazine (2.0 g, 10.7 mmol) in anhydrous DMF (15 mL) was cooled in an ice water bath, then NBS (3.0 g, 17.1 mmol) was added and the reaction heated to 50° C. and stirred for 1 h. The reaction was cooled to 23° C., diluted with 1 M aqueous sodium thiosulfate solution, extracted with EtOAc (3×) and the combined organic layers washed with saturated aqueous NaHCO₃ solution. The organic layer was separated, dried over anhydrous Na₂SO₄, filtered and concentrated under reduced pressure. The residue was purified by silica gel column (5-20% EtOAc in heptane) to provide a white solid. This was suspended in heptane then heated to 40° C. stirred for 20 mins, then the mixture filtered to yield 3-bromo-4,6-dichloro-pyrazolo[1,5-a]pyrazine (1.78 g, yield: 62%) as a white solid. ¹H NMR (500 MHz, DMSO-d₆) δ=9.33-9.30 (m, 1H), 8.45 (s, 1H).

### 2. Synthesis of tert-butyl ((cis)-3-((3-bromo-6-chloropyrazolo[1,5-a]pyrazin-4-yl)oxy)-3-methylcyclobutyl)carbamate

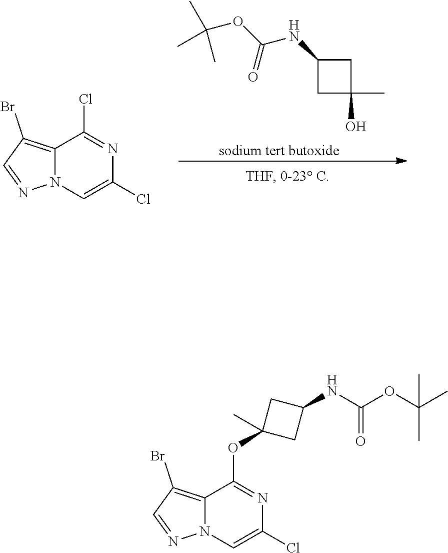 [SMILES: Clc1cn2ncc(Br)c2c(Cl)n1 ; CC(C)(C)OC(=O)N[C@H]1C[C@@](C)(O)C1 ; "sodium tert butoxide" ; "THF, 0-23° C." ; CC(C)(C)OC(=O)N[C@H]1C[C@@](C)(Oc2nc(Cl)cn3ncc(Br)c23)C1]

A flask containing cis tert-butyl N-(3-hydroxy-3-methyl-cyclobutyl)carbamate (820 mg, 4.07 mmol) in anhydrous THF (10 mL) was cooled in an ice water bath, then NaOtBu (637 mg, 6.63 mmol) was added in portions and the mixture stirred for 15 mins. 3-Bromo-4,6-dichloro-pyrazolo[1,5-a]pyrazine (1.02 g, 3.82 mmol) was added in portions, the mixture was allowed to warm to 23° C. and stirred for 30 mins. The reaction was quenched with water, then the biphasic mixture was extracted with EtOAc (3×). The combined organics were dried over anhydrous Na₂SO₄, filtered and concentrated under reduced pressure. The residue was purified by silica gel column (0-25% EtOAc in heptane) to afford tert-butyl ((cis)-3-((3-bromo-6-chloropyrazolo[1,5-a]pyrazin-4-yl)oxy)-3-methylcyclobutyl)carbamate as a white solid (1.34 g, yield: 81%). ¹H NMR (500 MHz, DMSO-d₆) δ=8.72 (s, 1H), 8.21 (s, 1H), 7.22 (br d, J=7.3 Hz, 1H), 3.84-3.74 (m, 1H), 2.66-2.62 (m, 2H), 2.38-2.33 (m, 2H), 1.66 (s, 3H), 1.36 (s, 9H).

### 3. Synthesis of tert-butyl ((cis)-3-((6-chloro-3-cyclopropylpyrazolo[1,5-a]pyrazin-4-yl)oxy)-3-methylcyclobutyl)carbamate

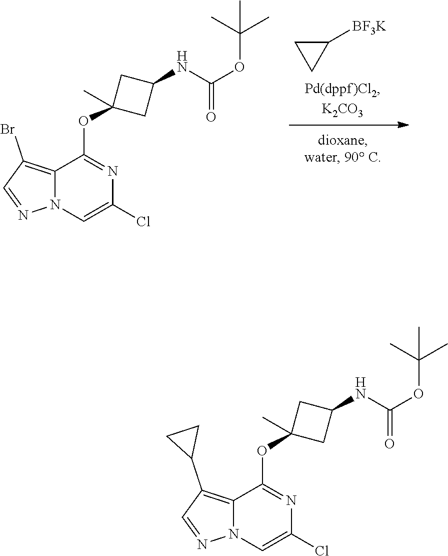

A vial containing tert-butyl ((cis)-3-((3-bromo-6-chloropyrazolo[1,5-a]pyrazin-4-yl)oxy)-3-methylcyclobutyl)carbamate (406 mg, 941 μmol), potassium cyclopropyltrifluoroborate (300 mg, 2.03 mmol), Pd(dppf)Cl₂:CH₂Cl₂ (161 mg, 197 μmol) and K₂CO₃ (389 mg, 2.81 mmol) in dioxane (4 mL) and water (0.4 mL) was degassed and backfilled with N₂ (3×) The heterogeneous reaction mixture was heated to 90° C. and stirred for 3 h, then cooled to RT. The mixture was partitioned between water and EtOAc, the layers separated and the aqueous phase extracted with EtOAc (2×). The combined organic extracts were washed with brine, then dried over anhydrous Na₂SO₄, filtered and concentrated under reduced pressure. The residue was purified by silica gel column (5-20% EtOAc in heptane) to afford tert-butyl ((cis)-3-((6-chloro-3-cyclopropylpyrazolo[1,5-a]pyrazin-4-yl)oxy)-3-methylcyclobutyl)carbamate as a colorless film (97 mg, yield: 26%). ¹H NMR (500 MHz, DMSO-d₆) δ=8.52 (s, 1H), 7.74 (s, 1H), 7.20 (br d, J=7.3 Hz, 1H), 3.79 (sxt, J=7.8 Hz, 1H), 2.64 (ddd, J=2.7, 7.5, 9.9 Hz, 2H), 2.37-2.32 (m, 2H), 2.22-2.17 (m, 1H), 1.68 (s, 3H), 1.36 (s, 9H), 1.00-0.96 (m, 2H), 0.73-0.70 (m, 2H).

### 4. Synthesis of tert-butyl ((cis)-3-((3-cyclopropyl-6-(1-methyl-1H-pyrazol-4-yl)pyrazolo[1,5-a]pyrazin-4-yl)oxy)-3-methylcyclobutyl)carbamate

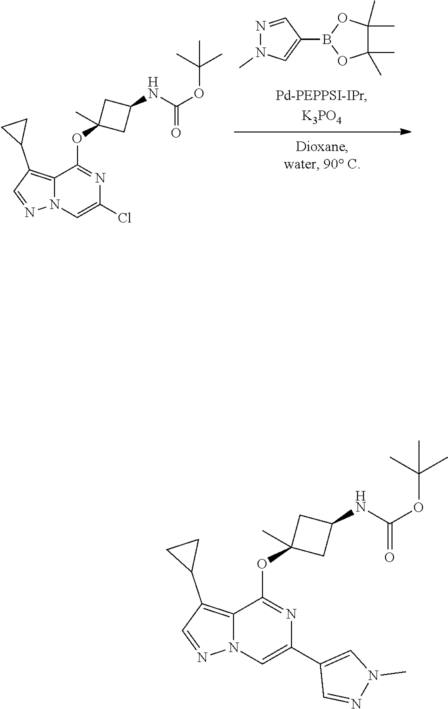

tert-Butyl ((cis)-3-((3-cyclopropyl-6-(1-methyl-1H-pyrazol-4-yl)pyrazolo[1,5-a]pyrazin-4-yl)oxy)-3-methylcyclobutyl)carbamate was obtained as a white solid, 102 mg, 94% from tert-butyl ((cis)-3-((6-chloro-3-cyclopropylpyrazolo[1,5-a]pyrazin-4-yl)oxy)-3-methylcyclobutyl)carbamate and 1-methyl-4-(4,4,5,5-tetramethyl-1,3,2-dioxaborolan-2-yl)pyrazole following the procedure described in step 4, Example 157. ¹H NMR (500 MHz, DMSO-d₆) δ=8.56 (s, 1H), 8.11 (s, 1H), 7.94 (s, 1H), 7.64 (s, 1H), 7.22 (br d, J=7.9 Hz, 1H), 3.88 (s, 3H), 3.87-3.80 (m, 1H), 2.79-2.73 (m, 2H), 2.39-2.33 (m, 2H), 2.24-2.19 (m, 1H), 1.73 (s, 3H), 1.37 (s, 9H), 0.99-0.95 (m, 2H), 0.72-0.69 (m, 2H).

### 5. Synthesis of (cis)-3-((3-cyclopropyl-6-(1-methyl-1H-pyrazol-4-yl)pyrazolo[1,5-a]pyrazin-4-yl)oxy)-3-methylcyclobutan-1-amine

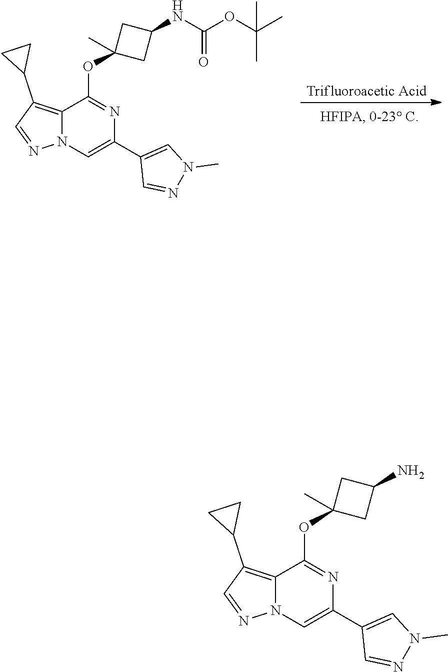 [SMILES: Cn1cc(-c2cn3ncc(C4CC4)c3c(O[C@]3(C)C[C@H](NC(=O)OC(C)(C)C)C3)n2)cn1 ; "Trifluoroacetic Acid" ; "HFIPA, 0-23° C." ; Cn1cc(-c2cn3ncc(C4CC4)c3c(O[C@]3(C)C[C@H](N)C3)n2)cn1]

(Cis)-3-((3-cyclopropyl-6-(1-methyl-1H-pyrazol-4-yl)pyrazolo[1,5-a]pyrazin-4-yl)oxy)-3-methylcyclobutan-1-amine trifluoroacetate was obtained as a colorless film in quantitative yield, from tert-butyl ((1s,3s)-3-((3-cyclopropyl-6-(1-methyl-1H-pyrazol-4-yl)pyrazolo[1,5-a]pyrazin-4-yl)oxy)-3-methylcyclobutyl)carbamate following the procedure described in step 2, Example 164. ESI-MS (M+H)⁺: 339.1.

### 5. Synthesis of N-((cis)-3-((3-cyclopropyl-6-(1-methyl-1H-pyrazol-4-yl)pyrazolo[1,5-a]pyrazin-4-yl)oxy)-3-methylcyclobutyl)acrylamide

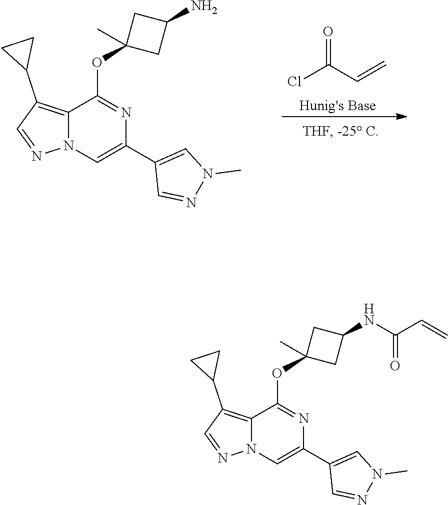

N-((cis)-3-((3-Cyclopropyl-6-(1-methyl-1H-pyrazol-4-yl)pyrazolo[1,5-a]pyrazin-4-yl)oxy)-3-methylcyclobutyl)acrylamide was obtained, 68 mg, 72% yield from (cis)-3-((3-cyclopropyl-6-(1-methyl-1H-pyrazol-4-yl)pyrazolo[1,5-a]pyrazin-4-yl)oxy)-3-methylcyclobutan-1-amine trifluoroacetate and acryloyl chloride, following the procedure described in step 3, Example 165. ESI-MS (M+H)⁺: 393.1. ¹H NMR (500 MHz, DMSO-d₆) δ=8.58 (s, 1H), 8.44 (d, J=7.9 Hz, 1H), 8.13 (s, 1H), 7.95 (s, 1H), 7.65 (s, 1H), 6.17-6.05 (m, 2H), 5.59 (dd, J=2.7, 9.5 Hz, 1H), 4.25-4.16 (m, 1H), 3.88 (s, 3H), 2.86 (ddd, J=2.7, 7.5, 9.9 Hz, 2H), 2.44-2.39 (m, 2H), 2.26-2.21 (m, 1H), 1.78 (s, 3H), 1.01-0.97 (m, 2H), 0.73-0.69 (m, 2H).

### Examples 167 & 168: 1-((1R,3R,5S)-3-((6-(1-methyl-1H-pyrazol-4-yl)pyrazolo[1,5-a]pyrazin-4-yl)oxy)-6-azabicyclo[3.2.1]octan-6-yl)prop-2-en-1-one and 1-((1S,3S,5R)-3-((6-(1-methyl-1H-pyrazol-4-yl)pyrazolo[1,5-a]pyrazin-4-yl)oxy)-6-azabicyclo[3.2.1]octan-6-yl)prop-2-en-1-one

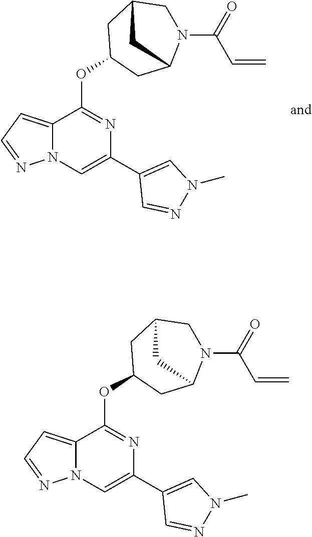 [SMILES: C=CC(=O)N1C[C@H]2C[C@@H](Oc3nc(-c4cnn(C)c4)cn4nccc34)C[C@@H]1C2 ; C=CC(=O)N1C[C@@H]2C[C@H](Oc3nc(-c4cnn(C)c4)cn4nccc34)C[C@H]1C2]

### 1. Synthesis of 1-((trans)-3-((6-(1-methyl-1H-pyrazol-4-yl)pyrazolo[1,5-a]pyrazin-4-yl)oxy)-6-azabicyclo[3.2.1]octan-6-yl)prop-2-en-1-one

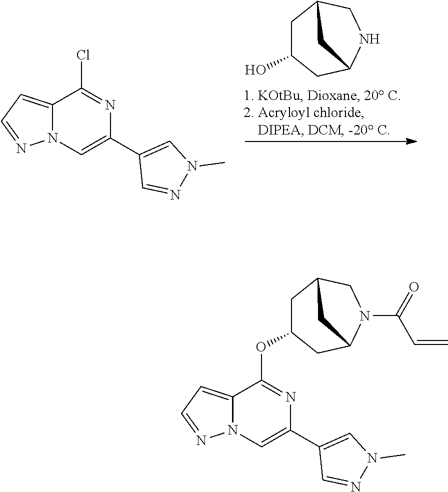

To a solution of 4-chloro-6-(1-methylpyrazol-4-yl)pyrazolo[1,5-a]pyrazine (Intermediate A, 200 mg, 856 μmol) and 6-azabicyclo[3.2.1]octan-3-ol (120 mg, 944 μmol) in dioxane (1.50 mL) was added KOtBu (1 M, 1 mL) and the reaction stirred at RT for 5 mins. The mixture was evaporated under reduced pressure and the residue treated with DCM (1.50 mL) and DIPEA (332 mg, 2.57 mmol). This mixture was stirred for 5 mins, cooled to −20° C., acryloyl chloride (77.5 mg, 856 μmol) added and the reaction stirred for 10 mins. To the cooled reaction was added NaHCO₃ (10 mL), the mixture warmed to RT while stirring vigorously. The phases were separated, the aqueous layer was extracted with DCM (2×10 mL) and the combined organic layers were concentrated to dryness. The crude reaction mixture was purified via silica gel column chromatography (heptane to 3:1 EtOAc:EtOH) to give 1-((trans)-3-((6-(1-methyl-1H-pyrazol-4-yl)pyrazolo[1,5-a]pyrazin-4-yl)oxy)-6-azabicyclo[3.2.1]octan-6-yl)prop-2-en-1-one (50 mg, 14% yield). LCMS m/z=379.3 (M+H)⁺. ¹H NMR (500 MHz, DMSO-d₆) δ ppm 1.39-1.66 (m, 1H) 1.74-1.90 (m, 3H) 2.21-2.38 (m, 1H) 2.60-3.07 (m, 2H) 3.38-3.60 (m, 1H) 3.62-3.70 (m, 1H) 3.88 (d, J=11.60 Hz, 3H) 4.34-4.54 (m, 1H) 5.31-5.44 (m, 1H) 5.76 (ddd, J=10.22, 5.95, 2.75 Hz, 1H) 6.32 (ddd, J=16.48, 8.55, 2.44 Hz, 1H) 6.61-6.82 (m, 2H) 7.86-8.12 (m, 3H) 8.70-8.78 (m, 1H).

### 2. Isolation of 1-((1R,3R,5S)-3-((6-(1-methyl-1H-pyrazol-4-yl)pyrazolo[1,5-a]pyrazin-4-yl)oxy)-6-azabicyclo[3.2.1]octan-6-yl)prop-2-en-1-one and 1-((1S,3S,5R)-3-((6-(1-methyl-1H-pyrazol-4-yl)pyrazolo[1,5-a]pyrazin-4-yl)oxy)-6-azabicyclo[3.2.1]octan-6-yl)prop-2-en-1-one

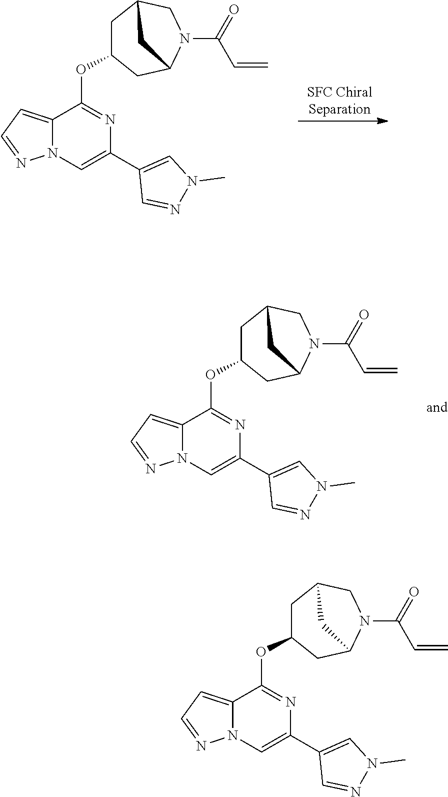 [SMILES: C=CC(=O)N1C[C@H]2C[C@@H](Oc3nc(-c4cnn(C)c4)cn4nccc34)C[C@@H]1C2 ; "SFC Chiral Separation" ; C=CC(=O)N1C[C@H]2C[C@@H](Oc3nc(-c4cnn(C)c4)cn4nccc34)C[C@@H]1C2 ; C=CC(=O)N1C[C@@H]2C[C@H](Oc3nc(-c4cnn(C)c4)cn4nccc34)C[C@H]1C2]

50 mg of the racemic material was resolved by chiral SFC purification (CHIRALPAK IB 30×250 mm, 5 um, 20% EtOH in CO₂, flow rate: 100 mL/min, ABPR 120 bar, MBPR 40 psi, column temp 40° C.) to afford two products as a first eluting peak (Example 167), Peak 1 (20.9 mg, 41%) and a second eluting peak (Example 168), Peak 2 (21.6 mg, 41.2%). ESI-MS (M+H)⁺: 379.3

### Examples 169, 170 171 & 172. 1-((1R,5S,6R)-7,7-dimethyl-6-((6-(1-methyl-1H-pyrazol-4-yl)pyrazolo[1,5-a]pyrazin-4-yl)oxy)-2-azabicyclo[3.2.0]heptan-2-yl)prop-2-en-1-one, 1-((1S,5R,6S)-7,7-dimethyl-6-((6-(1-methyl-1H-pyrazol-4-yl)pyrazolo[1,5-a]pyrazin-4-yl)oxy)-2-azabicyclo[3.2.0]heptan-2-yl)prop-2-en-1-one, 1-((1S,5R,6R)-7,7-dimethyl-6-((6-(1-methyl-1H-pyrazol-4-yl)pyrazolo[1,5-a]pyrazin-4-yl)oxy)-2-azabicyclo[3.2.0]heptan-2-yl)prop-2-en-1-one and 1-((1R,5S,6S)-7,7-dimethyl-6-((6-(1-methyl-1H-pyrazol-4-yl)pyrazolo[1,5-a]pyrazin-4-yl)oxy)-2-azabicyclo[3.2.0]heptan-2-yl)prop-2-en-1-one

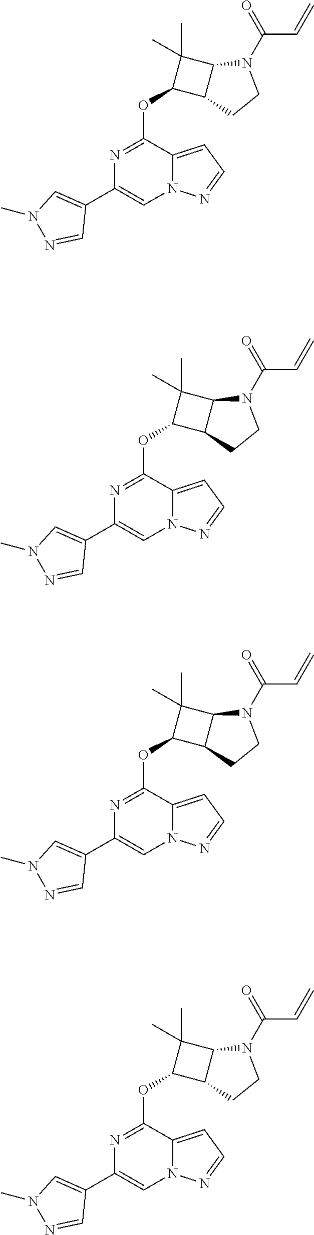

### 1. Synthesis of tert-butyl 7,7-dimethyl-6-((6-(1-methyl-1H-pyrazol-4-yl)pyrazolo[1,5-a]pyrazin-4-yl)oxy)-2-azabicyclo[3.2.0]heptane-2-carboxylate

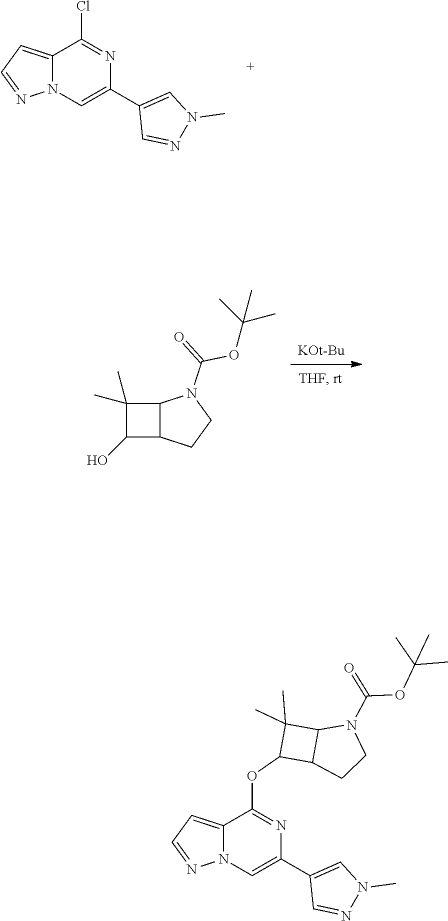 [SMILES: Cn1cc(-c2cn3nccc3c(Cl)n2)cn1 ; CC(C)(C)OC(=O)N1CCC2C(O)C(C)(C)C21 ; "THF, rt" ; Cn1cc(-c2cn3nccc3c(OC3C4CCN(C(=O)OC(C)(C)C)C4C3(C)C)n2)cn1 ; "KOt-Bu"]

To a solution of 4-chloro-6-(1-methylpyrazol-4-yl)pyrazolo[1,5-a]pyrazine (Intermediate A, 310 mg, 1.33 mmol) and tert-butyl 6-hydroxy-7,7-dimethyl-2-azabicyclo[3.2.0]heptane-2-carboxylate (320 mg, 1.33 mmol) in THF (2 mL) was added KOtBu (1 M, 2.50 mL) and the reaction stirred at RT for 1 h. The reaction was diluted with MTBE (5 mL) and washed with NaHCO₃ (5 mL). The aqueous layer was extracted with MTBE (10 mL) and the combined organic layers were concentrated to dryness. The residue was purified by column chromatography (heptane to EtOAc) to give tert-butyl 7,7-dimethyl-6-((6-(1-methyl-1H-pyrazol-4-yl)pyrazolo[1,5-a]pyrazin-4-yl)oxy)-2-azabicyclo[3.2.0]heptane-2-carboxylate in quantitative yield. LCMS m/z=439.2 (M+H)⁺.

### 2. Synthesis of 4-((7,7-dimethyl-2-azabicyclo[3.2.0]heptan-6-yl)oxy)-6-(1-methyl-1H-pyrazol-4-yl)pyrazolo[1,5-a]pyrazine

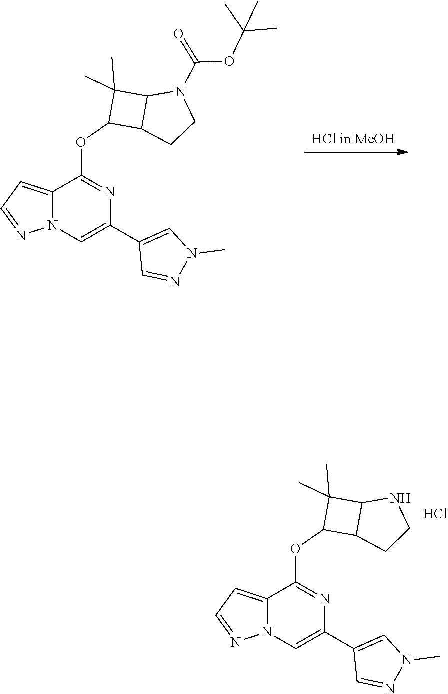 [SMILES: Cn1cc(-c2cn3nccc3c(OC3C4CCN(C(=O)OC(C)(C)C)C4C3(C)C)n2)cn1 ; "HCl in MeOH" ; Cn1cc(-c2cn3nccc3c(OC3C4CCNC4C3(C)C)n2)cn1 ; Cl]

To tert-butyl 7,7-dimethyl-6-((6-(1-methyl-1H-pyrazol-4-yl)pyrazolo[1,5-a]pyrazin-4-yl)oxy)-2-azabicyclo[3.2.0]heptane-2-carboxylate (648 mg, 1.48 mmol) was added HCl (1.25 M in MeOH, 8.9 mL) and the reaction stirred at RT overnight. The reaction mixture was stirred at 45° C. for 1 h then evaporated under reduced pressure to give 4-((7,7-dimethyl-2-azabicyclo[3.2.0]heptan-6-yl)oxy)-6-(1-methyl-1H-pyrazol-4-yl)pyrazolo[1,5-a]pyrazine, crude. ESI-MS (M+H)⁺: 339.2.

### 3. Synthesis of 1-(7,7-dimethyl-6-((6-(1-methyl-1H-pyrazol-4-yl)pyrazolo[1,5-a]pyrazin-4-yl)oxy)-2-azabicyclo[3.2.0]heptan-2-yl)prop-2-en-1-one

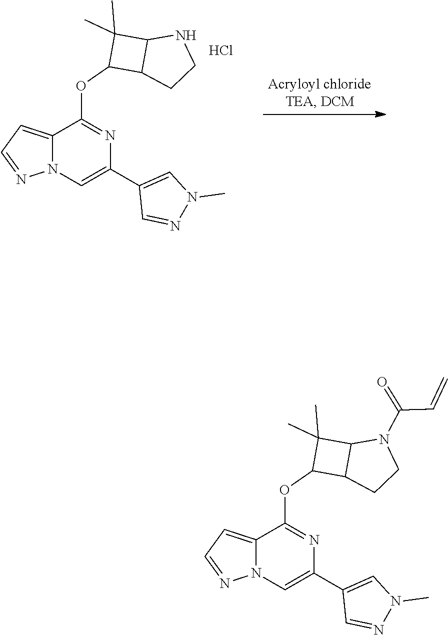 [SMILES: Cn1cc(-c2cn3nccc3c(OC3C4CCNC4C3(C)C)n2)cn1 ; Cl ; "Acryloyl chlorideTEA, DCM" ; C=CC(=O)N1CCC2C(Oc3nc(-c4cnn(C)c4)cn4nccc34)C(C)(C)C21]

4-((7,7-Dimethyl-2-azabicyclo[3.2.0]heptan-6-yl)oxy)-6-(1-methyl-1H-pyrazol-4-yl)pyrazolo[1,5-a]pyrazine was treated with DCM (10 mL) and TEA (787 mg, 7.78 mmol). This mixture was stirred for 10 mins, then cooled to −10° C. Acryloyl chloride (141 mg, 1.56 mmol) was added, the mixture stirred for 10 mins, then NaHCO₃ (10 mL) was added to quench the reaction and the mixture allowed to warm to RT. The layers were separated, the aqueous was extracted with EtOAc (10 mL) and the combined organic layers were concentrated to dryness and purified by column chromatography (heptane to EtOAc) to afford 425 mg (69% yield) of 1-(7,7-dimethyl-6-((6-(1-methyl-1H-pyrazol-4-yl)pyrazolo[1,5-a]pyrazin-4-yl)oxy)-2-azabicyclo[3.2.0]heptan-2-yl)prop-2-en-1-one LCMS m/z=393.1(M+H)⁺. ¹H NMR (500 MHz, DMSO-d₆) δ ppm 0.75 (d, J=10.38 Hz, 3H) 1.13-1.21 (m, 1H) 1.44 (d, J=10.99 Hz, 3H) 1.80-1.98 (m, 1H) 2.02-2.19 (m, 1H) 3.45-3.60 (m, 1H) 3.64-3.79 (m, 1H) 3.87-3.91 (m, 3H) 4.08-4.21 (m, 1H) 5.10-5.23 (m, 1H) 5.64-5.75 (m, 1H) 6.10-6.35 (m, 1H) 6.40-6.81 (m, 1H) 6.90-6.93 (m, 1H) 7.98-8.01 (m, 1H) 8.04 (s, 1H) 8.17 (s, 1H) 8.74-8.83 (m, 1H).

### 4. Separation of 1-(7,7-dimethyl-6-((6-(1-methyl-1H-pyrazol-4-yl)pyrazolo[1,5-a]pyrazin-4-yl)oxy)-2-azabicyclo[3.2.0]heptan-2-yl)prop-2-en-1-one

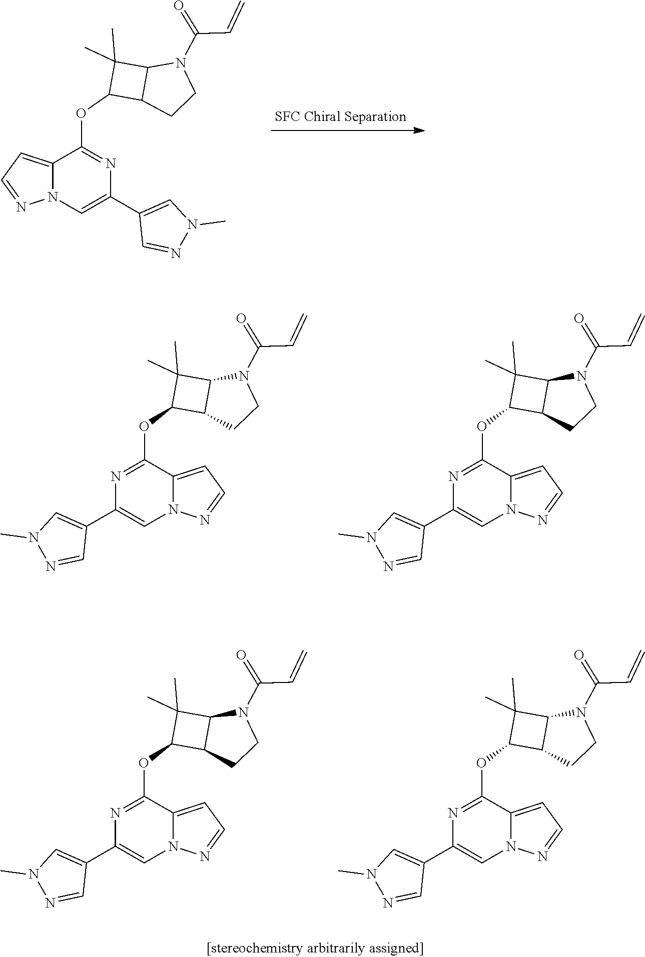 [SMILES: C=CC(=O)N1CCC2C(Oc3nc(-c4cnn(C)c4)cn4nccc34)C(C)(C)C21 ; "SFC Chiral Separation" ; C=CC(=O)N1CC[C@@H]2[C@@H](Oc3nc(-c4cnn(C)c4)cn4nccc34)C(C)(C)[C@@H]21 ; C=CC(=O)N1CC[C@@H]2[C@H]1C(C)(C)[C@H]2Oc1nc(-c2cnn(C)c2)cn2nccc12 ; C=CC(=O)N1CC[C@H]2[C@@H](Oc3nc(-c4cnn(C)c4)cn4nccc34)C(C)(C)[C@H]21 ; C=CC(=O)N1CC[C@@H]2[C@H](Oc3nc(-c4cnn(C)c4)cn4nccc34)C(C)(C)[C@@H]21]

1-(7,7-Dimethyl-6-((6-(1-methyl-1H-pyrazol-4-yl)pyrazolo[1,5-a]pyrazin-4-yl)oxy)-2-azabicyclo[3.2.0]heptan-2-yl)prop-2-en-1-one (450 mg) was resolved by chiral SFC purification (RegisPack 3×25 cm, 45% MeOH:MeCN 1:3 in CO₂, flow rate: 90 g/min, ABPR 100 bar, column temp 25° C.) to afford:

### Example 169: First eluting enantiomer (E1) Peak 1 (17 mg, 3.92%). LCMS m/z=393.1(M+H)⁺. ¹H NMR (500 MHz, DMSO-d₆) δ ppm 1.01-1.08 (m, 3H) 1.12-1.19 (m, 3H) 1.74-1.96 (m, 1H) 2.07-2.19 (m, 1H) 3.35-3.42 (m, 1H) 3.44-3.78 (m, 1H) 3.83-3.93 (m, 3H) 4.11-4.23 (m, 2H) 4.91-5.04 (m, 1H) 5.64-5.78 (m, 1H) 6.09-6.24 (m, 1H) 6.44-6.79 (m, 1H) 6.87 (d, J=1.22 Hz, 1H) 7.97-8.02 (m, 1H) 8.02-8.07 (m, 1H) 8.13-8.17 (m, 1H) 8.75-8.81 (m, 1H)

### Example 170: Second eluting enantiomer, (E2) Peak 2 (12 mg, 2.77%). LCMS m/z=393.1(M+H)⁺. ¹H NMR (500 MHz, DMSO-d6) δ ppm 0.99-1.08 (m, 3H) 1.12-1.19 (m, 3H) 1.74-1.95 (m, 1H) 2.05-2.15 (m, 1H) 3.35-3.40 (m, 1H) 3.45-3.79 (m, 1H) 3.84-3.93 (m, 3H) 4.10-4.23 (m, 2H) 4.93-5.05 (m, 1H) 5.66-5.74 (m, 1H) 6.12-6.24 (m, 1H) 6.44-6.79 (m, 1H) 6.84-6.90 (m, 1H) 7.96-8.01 (m, 1H) 8.02-8.07 (m, 1H) 8.12-8.17 (m, 1H) 8.74-8.80 (m, 1H)

And the third and fourth eluting enantiomer as a mixture of products. This was further resolved by chiral SFC purification ((RegisPack 3×25 cm, 40% i-PrOH:Hexane 1:1 in CO₂, flow rate: 80 g/min, ABPR 100 bar, column temp 25° C.) to afford:

### Example 171: The third eluting enantiomer, (E3) Peak 3 (74 mg, 17.1%). LCMS m/z=393.1(M+H)+. ¹H NMR (500 MHz, DMSO-d₆) δ ppm 0.75 (d, J=10.38 Hz, 3H) 1.44 (d, J=10.99 Hz, 3H) 1.78-1.99 (m, 1H) 2.01-2.18 (m, 1H) 3.45-3.58 (m, 1H) 3.64-3.77 (m, 1H) 3.87-3.94 (m, 4H) 4.08-4.16 (m, 1H) 5.12-5.21 (m, 1H) 5.65-5.72 (m, 1H) 6.10-6.21 (m, 1H) 6.41-6.72 (m, 1H) 6.87-6.94 (m, 1H) 7.99 (s, 1H) 8.03-8.06 (m, 1H) 8.17 (d, J=1.83 Hz, 1H) 8.77 (d, J=1.83 Hz, 1H)

### Example 172: And the fourth eluting enantiomer (78 mg, 17.9%). LCMS m/z=393.1(M+H)+. ¹H NMR (500 MHz, DMSO-d₆) δ ppm 0.75 (d, J=10.38 Hz, 3H) 1.41-1.47 (m, 3H) 1.80-1.99 (m, 1H) 2.01-2.17 (m, 1H) 3.46-3.58 (m, 1H) 3.64-3.78 (m, 1H) 3.86-3.95 (m, 4H) 4.06-4.17 (m, 1H) 5.11-5.22 (m, 1H) 5.64-5.73 (m, 1H) 6.10-6.21 (m, 1H) 6.41-6.72 (m, 1H) 6.91 (dd, J=3.66, 2.44 Hz, 1H) 7.99 (s, 1H) 8.03-8.06 (m, 1H) 8.17 (d, J=1.83 Hz, 1H) 8.74-8.80 (m, 1H)

Each sample was re-purified by silica gel column chromatography (heptane to 3:1 EtOAc:EtOH).

### Example 173: N-methyl-N-((1R,3S)-3-(6-(1-methylpyrazol-4-yl)-3-phenyl-pyrazolo[1,5-a]pyrazin-4-yl)oxycyclopentyl)prop-2-enamide

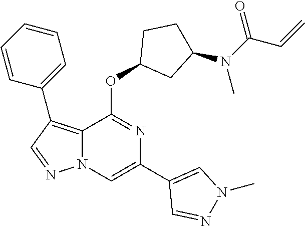 [SMILES: C=CC(=O)N(C)[C@@H]1CC[C@H](Oc2nc(-c3cnn(C)c3)cn3ncc(-c4ccccc4)c23)C1]

### 1. Synthesis of tert-butyl ((1R,3S)-3-((3-iodo-6-(1-methyl-1H-pyrazol-4-yl)pyrazolo[1,5-a]pyrazin-4-yl)oxy)cyclopentyl)(methyl)carbamate

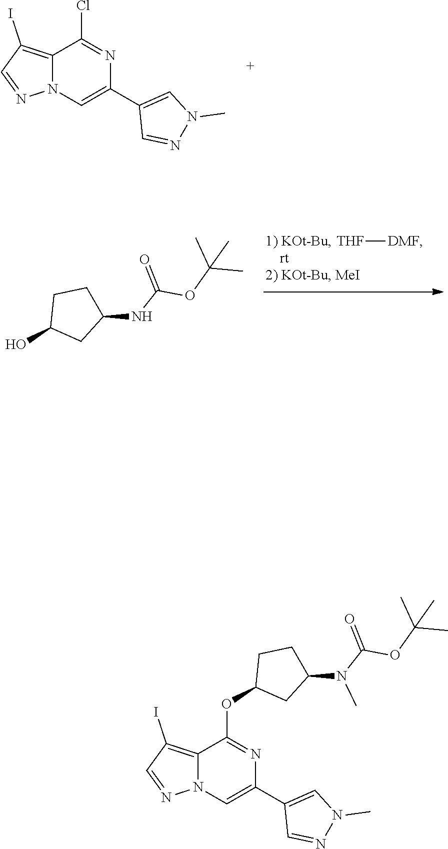

To a solution of 4-chloro-3-iodo-6-(1-methylpyrazol-4-yl)pyrazolo[1,5-a]pyrazine (Step 1, Example 59, 425 mg, 1.18 mmol) and tert-butyl N-((1R,3S)-3-hydroxycyclopentyl)carbamate (238 mg, 1.18 mmol) in THF (3 mL) and DMF (3 mL) was added KOtBu (1 M, 1.27 mL) and the reaction stirred at RT for 30 mins. Iodomethane (503 mg, 3.55 mmol) and KOtBu (1 M, 2.36 mL) were added and the reaction was stirred for 30 mins at RT. The reaction mixture was concentrated to a low volume, then diluted with water (10 mL) and extracted with MTBE (3×10 mL). The combined organic layers were concentrated to dryness, then purified by silica gel column chromatography (heptane to 50% EtOAc in heptane) to afford tert-butyl ((1R,3S)-3-((3-iodo-6-(1-methyl-1H-pyrazol-4-yl)pyrazolo[1,5-a]pyrazin-4-yl)oxy)cyclopentyl)(methyl)carbamate (430 mg, 68% yield). LCMS m/z=539.0 (M+H)⁺.

### 2. Synthesis of tert-butyl methyl((1R,3S)-3-((6-(1-methyl-1H-pyrazol-4-yl)-3-phenylpyrazolo[1,5-a]pyrazin-4-yl)oxy)cyclopentyl)carbamate

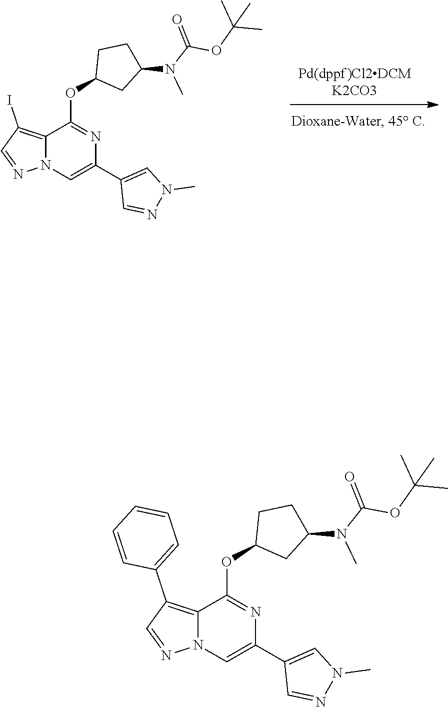 [SMILES: CN(C(=O)OC(C)(C)C)[C@@H]1CC[C@H](Oc2nc(-c3cnn(C)c3)cn3ncc(I)c23)C1 ; "Pd(dppf)Cl2·DCMK2CO3" ; CN(C(=O)OC(C)(C)C)[C@@H]1CC[C@H](Oc2nc(-c3cnn(C)c3)cn3ncc(-c4ccccc4)c23)C1]

A solution of tert-butyl ((1R,3S)-3-((3-iodo-6-(1-methyl-1H-pyrazol-4-yl)pyrazolo[1,5-a]pyrazin-4-yl)oxy)cyclopentyl)(methyl)carbamate (50 mg, 93 μmol), phenylboronic acid (15 mg, 123 μmol), K₂CO₃ (50 mg, 362 μmol) and Pd(dppf)Cl₂:DCM (5 mg, 6.12 μmol) in dioxane (1 mL) and water (1 mL) was stirred at 45° C. for 30 mins. The mixture was diluted with NaHCO₃ (10 mL) and extracted with EtOAc (3×10 mL). The combined organic layers were concentrated to dryness and purified by column chromatography (heptane to EtOAc) to afford tert-butyl methyl((1R,3S)-3-((6-(1-methyl-1H-pyrazol-4-yl)-3-phenylpyrazolo[1,5-a]pyrazin-4-yl)oxy)cyclopentyl)carbamate (30 mg, 66% yield). ESI-MS (M+H)⁺: 427.2.

### 3. Synthesis of N-methyl-N—((1R,3S)-3-((6-(1-methyl-1H-pyrazol-4-yl)-3-phenylpyrazolo[1,5-a]pyrazin-4-yl)oxy)cyclopentyl)acrylamide

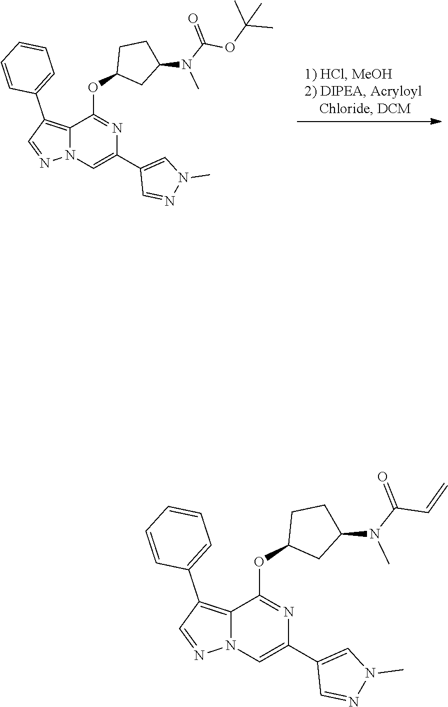

To tert-butyl methyl((1R,3S)-3-((6-(1-methyl-1H-pyrazol-4-yl)-3-phenylpyrazolo[1,5-a]pyrazin-4-yl)oxy)cyclopentyl)carbamate (30 mg, 61.4 μmol) was added HCl (1.25 M in MeOH, 3 mL) and the reaction was left to stand at 30° C. overnight. The reaction was concentrated to dryness, diluted with EtOAc (5 mL) and concentrated to give a white solid. DCM (3 mL) and DIPEA (42.4 mg, 328 μmol) were added and the solution cooled to −20° C. Acryloyl chloride (5.94 mg, 66 μmol) was added and the reaction stirred for 10 mins. The mixture was purified directly by silica gel column chromatography (heptane to 3:1 EtOAc:EtOH) and re-purified by prep-HPLC using a Waters Sunfire Prep C18, 5 μm, 19 mm×100 mm column with mobile phase H₂O (A) and MeCN (B) and a gradient of 5-60% B (0.2% NH₄HCO₃ final v/v % modifier) with flow rate at 30 mL/min to afford N-methyl-N-((1R,3S)-3-((6-(1-methyl-1H-pyrazol-4-yl)-3-phenylpyrazolo[1,5-a]pyrazin-4-yl)oxy)cyclopentyl)acrylamide (16.2 mg, 52% yield). LCMS m/z=465.2 (M+Na)⁺. ¹H NMR (500 MHz, DMSO-d₆) δ ppm 1.57-1.83 (m, 4H) 1.93-2.02 (m, 2H) 2.44-2.59 (d, 3H) 3.90 (s, 3H) 4.47-5.06 (m, 1H) 5.61-5.71 (m, 2H) 6.05 (br t, J=16.18 Hz, 1H) 6.56-6.88 (m, 1H) 7.33-7.40 (m, 1H) 7.43-7.47 (m, 2H) 7.65-7.70 (m, 2H) 8.02-8.07 (m, 1H) 8.16-8.20 (m, 1H) 8.22-8.26 (m, 1H) 8.75-8.79 (m, 1H).

### Example 174: 1-((3aR,5s,6aS)-5-((6-(1-methyl-1H-pyrazol-4-yl)pyrazolo[1,5-a]pyrazin-4-yl)oxy)hexahydrocyclopenta[c]pyrrol-2(1H)-yl)prop-2-en-1-one

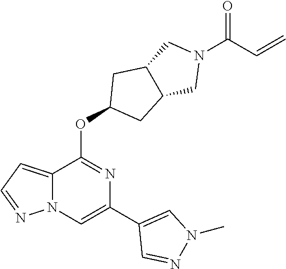 [SMILES: C=CC(=O)N1C[C@H]2C[C@H](Oc3nc(-c4cnn(C)c4)cn4nccc34)C[C@H]2C1]

### 1. Synthesis of 1-((3aR,5s,6aS)-5-((6-(1-methyl-1H-pyrazol-4-yl)pyrazolo[1,5-a]pyrazin-4-yl)oxy)hexahydrocyclopenta[c]pyrrol-2(1H)-yl)prop-2-en-1-one

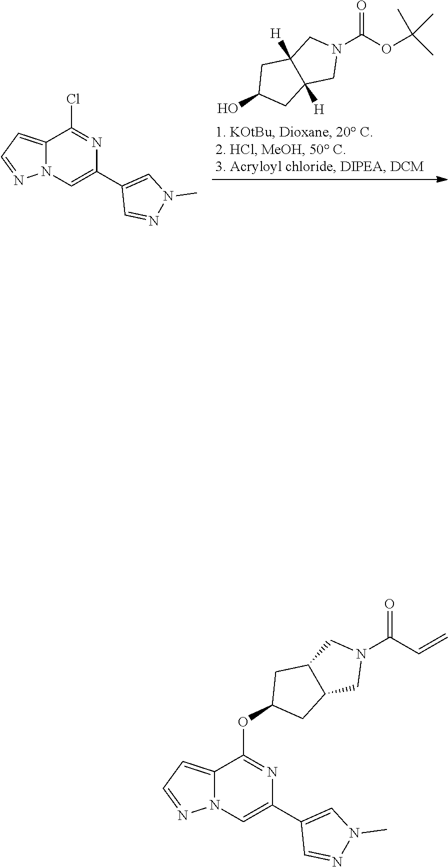 [SMILES: Cn1cc(-c2cn3nccc3c(Cl)n2)cn1 ; CC(C)(C)OC(=O)N1C[C@H]2C[C@H](O)C[C@H]2C1 ; C=CC(=O)N1C[C@H]2C[C@H](Oc3nc(-c4cnn(C)c4)cn4nccc34)C[C@H]2C1]

To a solution of 4-chloro-6-(1-methylpyrazol-4-yl)pyrazolo[1,5-a]pyrazine (Intermediate A, 100 mg, 428 μmol) and tert-butyl (3aS,6aR)-5-hydroxy-3,3a,4,5,6,6a-hexahydro-1H-cyclopenta[c]pyrrole-2-carboxylate (100 mg, 440 μmol) in dioxane (3 mL) was added KOtBu (1 M, 500 μL) and the reaction stirred at RT for 5 mins. The reaction mixture was concentrated to dryness, then HCl (1.25 M, 3.5 mL) added and the solution stirred at 50° C. for 3 h. The mixture was concentrated to dryness, diluted with EtOAc (10 mL) and concentrated to give a white solid. This was treated with DCM (5 mL) and DIPEA (166 mg, 1.28 mmol), then cooled to −20 C while stirring. Acryloyl chloride (39 mg, 428 μmol) was added and the reaction stirred for 5 mins. The crude reaction mixture was purified directly by silica gel column chromatography (heptane to 3:1 EtOAc:EtOH) to give 1-((3aR,5s,6aS)-5-((6-(1-methyl-1H-pyrazol-4-yl)pyrazolo[1,5-a]pyrazin-4-yl)oxy)hexahydrocyclopenta[c]pyrrol-2(1H)-yl)prop-2-en-1-one (155 mg, 96% yield). ESI-MS (M+H)⁺: 379.1. ¹H NMR (DMSO-d₆) δ: 8.73 (d, J=1.2 Hz, 1H), 8.18 (s, 1H), 8.00 (d, J=1.8 Hz, 2H), 6.81 (d, J=3.1 Hz, 1H), 6.62 (dd, J=16.5, 10.4 Hz, 1H), 6.14 (dd, J=17.1, 2.4 Hz, 1H), 5.77 (dt, J=6.0, 2.8 Hz, 1H), 5.64-5.71 (m, 1H), 3.88 (s, 3H), 3.71-3.81 (m, 1H), 3.56-3.65 (m, 1H), 3.46-3.54 (m, 1H), 3.34-3.41 (m, 1H), 2.91-3.03 (m, 1H), 2.86 (br d, J=6.1 Hz, 1H), 2.10-2.20 (m, 2H), 2.00-2.09 (m, 2H)

### Examples 175 & 176: N-methyl-N-((1S,3R)-3-((6-(1-methyl-1H-pyrazol-4-yl)pyrazolo[1,5-a]pyrazin-4-yl)oxy)cyclohexyl)acrylamide and N-methyl-N-((1R,3S)-3-((6-(1-methyl-1H-pyrazol-4-yl)pyrazolo[1,5-a]pyrazin-4-yl)oxy)cyclohexyl)acrylamide

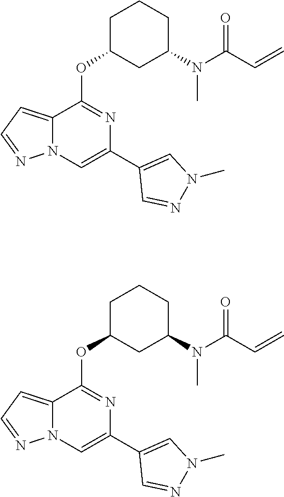 [SMILES: C=CC(=O)N(C)[C@H]1CCC[C@@H](Oc2nc(-c3cnn(C)c3)cn3nccc23)C1 ; C=CC(=O)N(C)[C@@H]1CCC[C@H](Oc2nc(-c3cnn(C)c3)cn3nccc23)C1]

### 1. Synthesis of rac-tert-butyl ((1S,3R)-3-hydroxycyclohexyl)carbamate

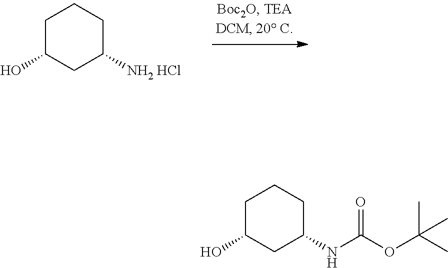

To a slurry of (rac)-(1S,3R)-aminocyclohexane-1-ol hydrochloride (1 g, 6.59 mmol) in DCM (20 mL) and TEA (1.33 g, 13.19 mmol) was added a solution of Boc anhydride (1.44 g, 6.59 mmol) in DCM (5 mL) and the reaction stirred at 35° C. overnight. The reaction was purified directly via silica gel column chromatography (EtOAc to 3:1 EtOAc:EtOH) to give (rac)-tert-butyl ((1S,3R)-3-hydroxycyclohexyl)carbamate as a white solid (1.31 g, 93% yield). ¹H NMR (DMSO-d₆) δ: 6.72 (br d, J=7.9 Hz, 1H), 4.57 (d, J=4.3 Hz, 1H), 3.35 (ddd, J=15.0, 6.4, 4.3 Hz, 1H), 3.20 (br dd, J=7.6, 3.4 Hz, 1H), 1.90 (br d, J=11.6 Hz, 1H), 1.73 (br d, J=11.6 Hz, 1H), 1.53-1.69 (m, 2H), 1.37 (s, 9H), 1.09-1.22 (m, 1H), 0.87-1.08 (m, 3H).

### 2. Synthesis of rac-tert-butyl ((1R,3S)-3-((6-(1-methyl-1H-pyrazol-4-yl)pyrazolo[1,5-a]pyrazin-4-yl)oxy)cyclohexyl)carbamate

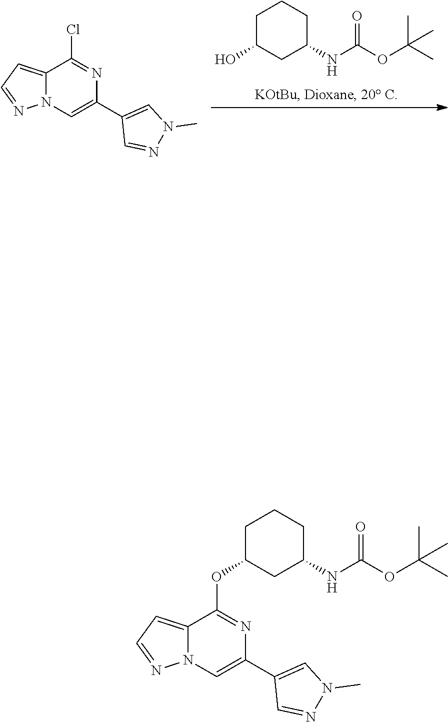 [SMILES: Cn1cc(-c2cn3nccc3c(Cl)n2)cn1 ; CC(C)(C)OC(=O)N[C@H]1CCC[C@@H](O)C1 ; Cn1cc(-c2cn3nccc3c(O[C@@H]3CCC[C@H](NC(=O)OC(C)(C)C)C3)n2)cn1]

To a solution of 4-chloro-6-(1-methylpyrazol-4-yl)pyrazolo[1,5-a]pyrazine (Intermediate A, 250 mg, 1.07 mmol) and (rac)-tert-butyl ((1R,3S)-3-hydroxycyclohexyl)carbamate (250 mg, 1.16 mmol) in THF (2 mL) was added KOtBu (1 M, 3.5 mL) and the reaction stirred at RT for 5 mins. The reaction mixture was diluted with NaHCO₃ (5 mL) and extracted with EtOAc (3×10 mL). The combined organic layers were concentrated to dryness and purified by silica gel column chromatography (heptane to 3:1 EtOAc:EtOH) to give (rac)-tert-butyl ((1R,3S)-3-((6-(1-methyl-1H-pyrazol-4-yl)pyrazolo[1,5-a]pyrazin-4-yl)oxy)cyclohexyl)carbamate (325 mg, 74% yield). ESI-MS (M+H)⁺: 413.2. ¹H NMR (DMSO-d₆) δ: 8.73 (d, J=0.8 Hz, 1H), 8.18 (s, 1H), 7.98-8.04 (m, 2H), 6.77-6.91 (m, 2H), 5.71 (br s, 1H), 3.88 (s, 3H), 3.64-3.79 (m, 1H), 2.11 (br d, J=14.3 Hz, 1H), 1.86-1.96 (m, 1H), 1.56-1.85 (m, 6H), 1.22-1.44 (m, 10H).

### 3. Synthesis of (rac)-(1S,3R)—N-methyl-3-((6-(1-methyl-1H-pyrazol-4-yl)pyrazolo[1,5-a]pyrazin-4-yl)oxy)cyclohexan-1-amine hydrochloride

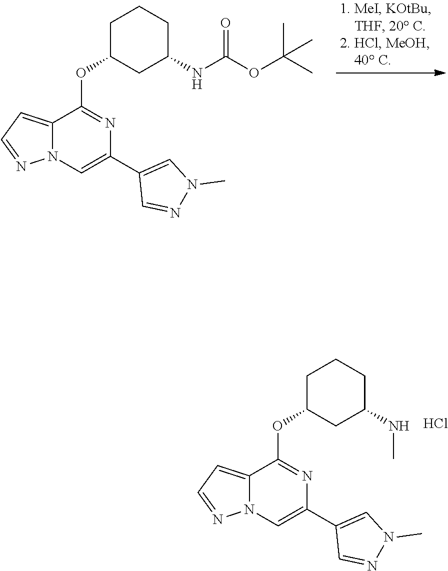 [SMILES: Cn1cc(-c2cn3nccc3c(O[C@@H]3CCC[C@H](NC(=O)OC(C)(C)C)C3)n2)cn1 ; CN[C@H]1CCC[C@@H](Oc2nc(-c3cnn(C)c3)cn3nccc23)C1 ; Cl]

To a vial was added (rac)-tert-butyl ((1S,3R)-3-((6-(1-methyl-1H-pyrazol-4-yl)pyrazolo[1,5-a]pyrazin-4-yl)oxy)cyclohexyl)carbamate (155 mg, 376 μmol), THF (3 mL), KOtBu (1 M, 1.55 mL) and iodomethane (213 mg, 1.50 mmol) which was stirred at 40° C. for 30 mins. The reaction was diluted with NaHCO₃ (10 mL) and extracted with EtOAc (3×10 mL). The combined organic layers were concentrated to dryness and purified by silica gel column chromatography (heptane to EtOAc) to give a white solid (100 mg, 63% yield). This was treated with 1.25 M HCl in MeOH (10 mL) and placed on a 40° C. hot plate for 16 h. The reaction was concentrated to dryness to give (rac)-(1S,3R)—N-methyl-3-((6-(1-methyl-1H-pyrazol-4-yl)pyrazolo[1,5-a]pyrazin-4-yl)oxy)cyclohexan-1-amine hydrochloride. ESI-MS (M+H)⁺: 327.1.

### 4. Synthesis of (rac)-N-methyl-N-((1S,3R)-3-((6-(1-methyl-1H-pyrazol-4-yl)pyrazolo[1,5-a]pyrazin-4-yl)oxy)cyclohexyl)acrylamide

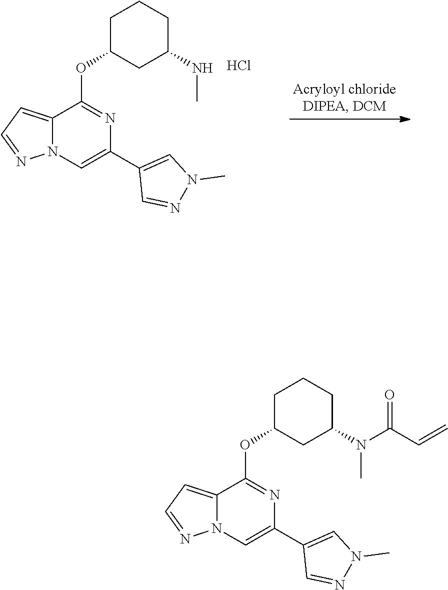 [SMILES: CN[C@H]1CCC[C@@H](Oc2nc(-c3cnn(C)c3)cn3nccc23)C1 ; Cl ; C=CC(=O)N(C)[C@H]1CCC[C@@H](Oc2nc(-c3cnn(C)c3)cn3nccc23)C1]

(rac)-(1S,3R)—N-methyl-3-((6-(1-methyl-1H-pyrazol-4-yl)pyrazolo[1,5-a]pyrazin-4-yl)oxy)cyclohexan-1-amine hydrochloride was treated with THF (3 mL) and TEA (101 mg, 1.00 mmol), then cooled to −20° C. and acryloyl chloride (18 mg, 200 μmol) added and the reaction stirred for 5 mins. NaHCO₃ (3 mL) was added, the mixture stirred and the layers separated. The aqueous phase was extracted with EtOAc (3×3 mL) and the combined organic layers concentrated to dryness. The product was purified by prep-HPLC using a Waters Sunfire Prep C18, 5 μm, 19 mm×100 mm column with mobile phase H₂O (A) and MeCN (B) and a gradient of 5-60% B (0.2% TFA final v/v % modifier) with flow rate at 30 m/min, to give (rac)-N-methyl-N-((1S,3R)-3-((6-(1-methyl-1H-pyrazol-4-yl)pyrazolo[1,5-a]pyrazin-4-yl)oxy)cyclohexyl)acrylamide (76 mg). ESI-MS (M+H)⁺: 381.2. ¹H NMR (DMSO-d₆) δ: 8.74 (s, 1H), 8.26 (s, 1H), 7.97-8.07 (m, 2H), 6.79 (s, 2H), 6.03-6.20 (m, 1H), 5.62-5.74 (m, 1H), 5.31-5.49 (m, 1H), 4.47-4.66 (m, 1H), 4.04-4.22 (m, 1H), 3.90 (d, J=3.7 Hz, 3H), 2.11-2.32 (m, 3H), 1.57 (br s, 10H).

### 5. Isolation of N-methyl-N-((1S,3R)-3-((6-(1-methyl-1H-pyrazol-4-yl)pyrazolo[1,5-a]pyrazin-4-yl)oxy)cyclohexyl)acrylamide and N-methyl-N—((1R,3S)-3-((6-(1-methyl-1H-pyrazol-4-yl)pyrazolo[1,5-a]pyrazin-4-yl)oxy)cyclohexyl)acrylamide

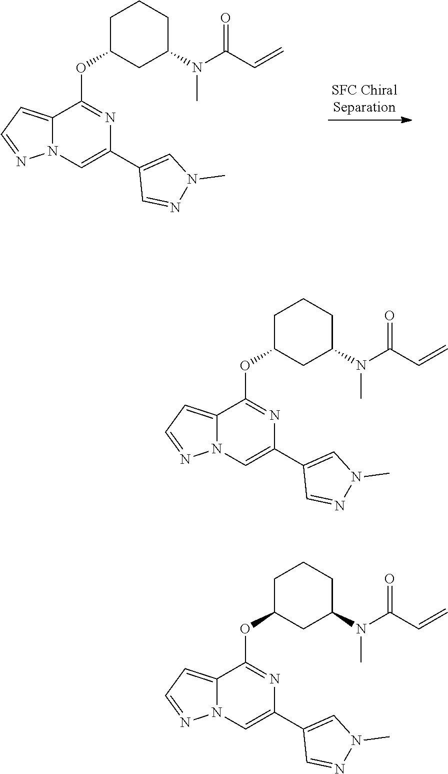 [SMILES: C=CC(=O)N(C)[C@H]1CCC[C@@H](Oc2nc(-c3cnn(C)c3)cn3nccc23)C1 ; C=CC(=O)N(C)[C@H]1CCC[C@@H](Oc2nc(-c3cnn(C)c3)cn3nccc23)C1 ; C=CC(=O)N(C)[C@@H]1CCC[C@H](Oc2nc(-c3cnn(C)c3)cn3nccc23)C1]

(rac)-N-Methyl-N-((cis)-3-((6-(1-methyl-1H-pyrazol-4-yl)pyrazolo[1,5-a]pyrazin-4-yl)oxy)cyclohexyl)acrylamide (68 mg) was resolved by chiral SFC purification (CHIRALPAK AD-H 30×250 mm, 5 μm, 40% MeOH in CO₂, flow rate: 100 mL/min, ABPR 120 bar, MBPR 40 psi, column temp 40° C.) to afford the two products as a first eluting peak (Example 175), Peak 1 (13 mg, 18.2%, Rt=2.55 min, 99.3% ee) and a second eluting peak (Example 176), Peak 2 (17 mg, 16.7%, Rt=3.52 min, 99.72% ee) as off-white solids. ESI-MS (M+H)⁺: 381.2.

Examples 177 & 178: N-methyl-N-((1S,3R)-3-((6-(1-methyl-1H-pyrazol-4-yl)pyrazolo[1,5-a]pyrazin-4-yl)oxy)cyclohexyl)but-2-ynamide and N-methyl-N-((1R,3S)-3-((6-(1-methyl-1H-pyrazol-4-yl)pyrazolo[1,5-a]pyrazin-4-yl)oxy)cyclohexyl)but-2-ynamide

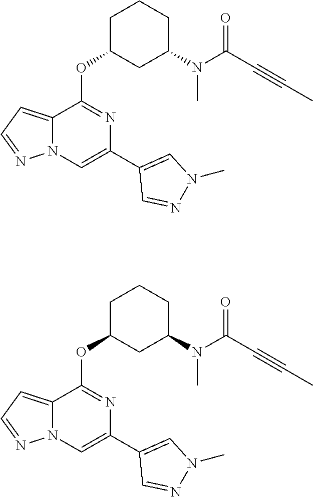 [SMILES: CC#CC(=O)N(C)[C@H]1CCC[C@@H](Oc2nc(-c3cnn(C)c3)cn3nccc23)C1 ; CC#CC(=O)N(C)[C@@H]1CCC[C@H](Oc2nc(-c3cnn(C)c3)cn3nccc23)C1]

### 1. Synthesis of (rac)-N-methyl-N—((1S,3R)-3-((6-(1-methyl-1H-pyrazol-4-yl)pyrazolo[1,5-a]pyrazin-4-yl)oxy)cyclohexyl)but-2-ynamide

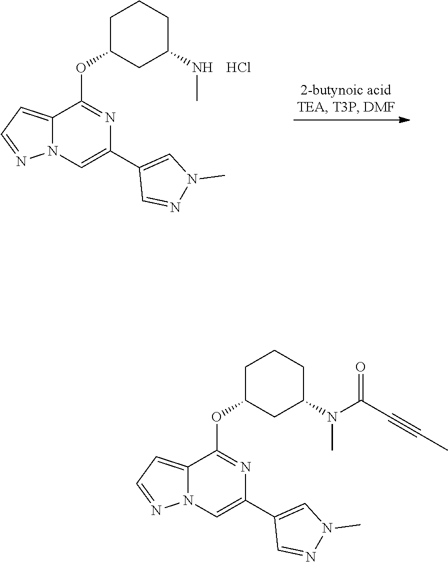 [SMILES: CN[C@H]1CCC[C@@H](Oc2nc(-c3cnn(C)c3)cn3nccc23)C1 ; Cl ; CC#CC(=O)N(C)[C@H]1CCC[C@@H](Oc2nc(-c3cnn(C)c3)cn3nccc23)C1]

(rac)-N-Methyl-N-((1S,3R)-3-((6-(1-methyl-1H-pyrazol-4-yl)pyrazolo[1,5-a]pyrazin-4-yl)oxy)cyclohexyl)but-2-ynamide was obtained from (rac)-(1S,3R)—N-methyl-3-((6-(1-methyl-1H-pyrazol-4-yl)pyrazolo[1,5-a]pyrazin-4-yl)oxy)cyclohexan-1-amine hydrochloride(step 3, Example 175) and but-2-ynoic acid, following the procedure described in step 3, Example 149 (70 mg, 89% yield). ESI-MS (M+H)⁺: 393.1. ¹H NMR (DMSO-d₆) δ: 8.74 (d, J=7.9 Hz, 1H), 8.18-8.24 (m, 1H), 8.00 (d, J=4.9 Hz, 2H), 6.79 (dd, J=7.3, 1.8 Hz, 1H), 5.29-5.43 (m, 1H), 4.33-4.50 (m, 1H), 3.89 (s, 3H), 3.06 (s, 2H), 2.76 (s, 2H), 2.14-2.31 (m, 3H), 2.11 (s, 2H), 2.01 (s, 2H), 1.37-1.95 (m, 9H).

### 2. Isolation of N-methyl-N-((1S,3R)-3-((6-(1-methyl-1H-pyrazol-4-yl)pyrazolo[1,5-a]pyrazin-4-yl)oxy)cyclohexyl)acrylamide and N-methyl-N—((1R,3S)-3-((6-(1-methyl-1H-pyrazol-4-yl)pyrazolo[1,5-a]pyrazin-4-yl)oxy)cyclohexyl)acrylamide

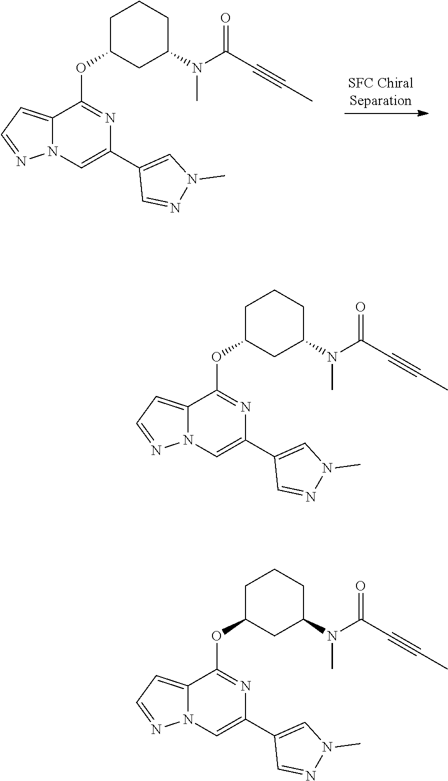 [SMILES: CC#CC(=O)N(C)[C@H]1CCC[C@@H](Oc2nc(-c3cnn(C)c3)cn3nccc23)C1 ; CC#CC(=O)N(C)[C@H]1CCC[C@@H](Oc2nc(-c3cnn(C)c3)cn3nccc23)C1 ; CC#CC(=O)N(C)[C@@H]1CCC[C@H](Oc2nc(-c3cnn(C)c3)cn3nccc23)C1]

(rac)-N-Methyl-N-((1S,3R)-3-((6-(1-methyl-1H-pyrazol-4-yl)pyrazolo[1,5-a]pyrazin-4-yl)oxy)cyclohexyl)but-2-ynamide (70 mg) was resolved by chiral SFC purification (CHIRALPAK IA-H 30×250 mm, 5 μm, 40% MeOH in CO₂, flow rate: 100 mL/min, ABPR 120 bar, MBPR 40 psi, column temp 40° C.) to afford the two products as a first eluting peak (Example 177), Peak 1 (10.2 mg, 14.6%, Rt=2.28 min, 99.93% ee) and a second eluting peak (Example 178), Peak 2 (9.5 mg, 13.6%, Rt=3.23 min, 99.98% ee) as off-white solids. ESI-MS (M+H)⁺: 393.1.

### Examples 179 & 180: N-((1S,3R)-3-((6-(1-methyl-1H-pyrazol-4-yl)pyrazolo[1,5-a]pyrazin-4-yl)oxy)cyclohexyl)but-2-ynamide and N-((1R,3S)-3-((6-(1-methyl-1H-pyrazol-4-yl)pyrazolo[1,5-a]pyrazin-4-yl)oxy)cyclohexyl)but-2-ynamide

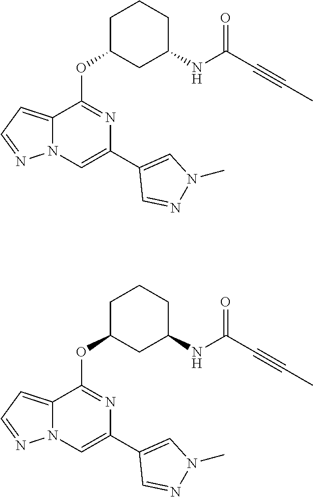 [SMILES: CC#CC(=O)N[C@H]1CCC[C@@H](Oc2nc(-c3cnn(C)c3)cn3nccc23)C1 ; CC#CC(=O)N[C@@H]1CCC[C@H](Oc2nc(-c3cnn(C)c3)cn3nccc23)C1]

### 1. Synthesis of rac-(1R,3S)-3-((6-(1-methyl-1H-pyrazol-4-yl)pyrazolo[1,5-a]pyrazin-4-yl)oxy)cyclohexan-1-amine hydrochloride

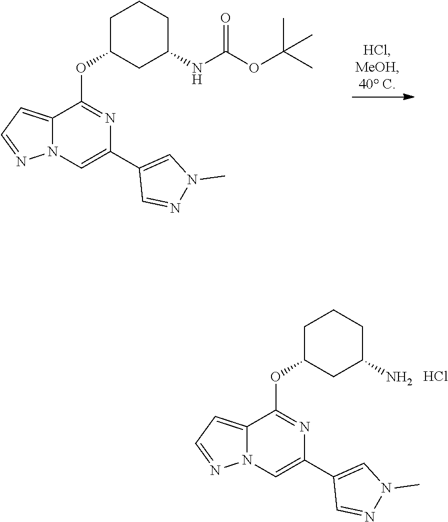 [SMILES: Cn1cc(-c2cn3nccc3c(O[C@@H]3CCC[C@H](NC(=O)OC(C)(C)C)C3)n2)cn1 ; Cn1cc(-c2cn3nccc3c(O[C@@H]3CCC[C@H](N)C3)n2)cn1 ; Cl]

To a 30 mL vial was added rac-tert-butyl ((1R,3S)-3-((6-(1-methyl-1H-pyrazol-4-yl)pyrazolo[1,5-a]pyrazin-4-yl)oxy)cyclohexyl)carbamate (155 mg, 0.376 mmol) and 5 mL 1.25 M HCl in MeOH and placed on a 40° C. hot plate for 16 hours. The reaction mixture was evaporated to dryness in vacuo and used without further purification. ESI-MS (M+H)⁺: 313.1.

### 2. Synthesis of rac-N-((1R,3S)-3-((6-(1-methyl-1H-pyrazol-4-yl)pyrazolo[1,5-a]pyrazin-4-yl)oxy)cyclohexyl)but-2-ynamide

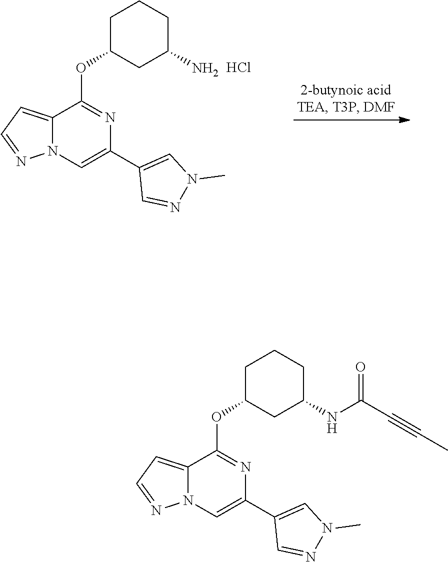 [SMILES: Cn1cc(-c2cn3nccc3c(O[C@@H]3CCC[C@H](N)C3)n2)cn1 ; Cl ; CC#CC(=O)N[C@H]1CCC[C@@H](Oc2nc(-c3cnn(C)c3)cn3nccc23)C1]

rac-(1R,3S)-3-((6-(1-methyl-1H-pyrazol-4-yl)pyrazolo[1,5-a]pyrazin-4-yl)oxy)cyclohexan-1-amine hydrochloride was treated with DMF (3 mL) and TEA (101 mg, 1.0 mmol) while stirring at RT. To this solution was added 2-butynoic acid (34 mg, 400 μmol) and T3P (191 mg, 300 μmol, 50% in DMF) which was left to stir for 16 h. The reaction mixture was diluted with NaHCO₃ (10 mL) and extracted with EtOAc (3×10 mL). The organic layers were combined and concentrated to dryness, then purified by prep-HPLC using a Waters Sunfire Prep C18, 5 μm, 19 mm×100 mm column with mobile phase H₂O (A) and MeCN (B) and a gradient of 5-60% B (0.2% NH₄HCO₃ final v/v % modifier) and flow rate at 30 mL/min to give rac-N-((1R,3S)-3-((6-(1-methyl-1H-pyrazol-4-yl)pyrazolo[1,5-a]pyrazin-4-yl)oxy)cyclohexyl)but-2-ynamide. (70 mg, 92.5% yield). ESI-MS (M+H)⁺: 379.1. ¹H NMR (DMSO-d₆) δ: 8.73 (s, 1H), 8.56 (d, J=7.9 Hz, 1H), 8.23 (s, 1H), 7.97-8.05 (m, 2H), 6.77-6.86 (m, 1H), 5.32 (tt, J=11.0, 4.3 Hz, 1H), 3.89 (s, 4H), 3.79-3.88 (m, 1H), 2.34-2.43 (m, 2H), 2.11-2.20 (m, 1H), 1.95 (s, 3H), 1.75-1.89 (m, 3H), 1.33-1.59 (m, 3H), 1.10-1.29 (m, 2H).

### 3. Isolation of N-methyl-N-((1S,3R)-3-((6-(1-methyl-1H-pyrazol-4-yl)pyrazolo[1,5-a]pyrazin-4-yl)oxy)cyclohexyl)acrylamide and N-methyl-N-((1R,3S)-3-((6-(1-methyl-1H-pyrazol-4-yl)pyrazolo[1,5-a]pyrazin-4-yl)oxy)cyclohexyl)acrylamide

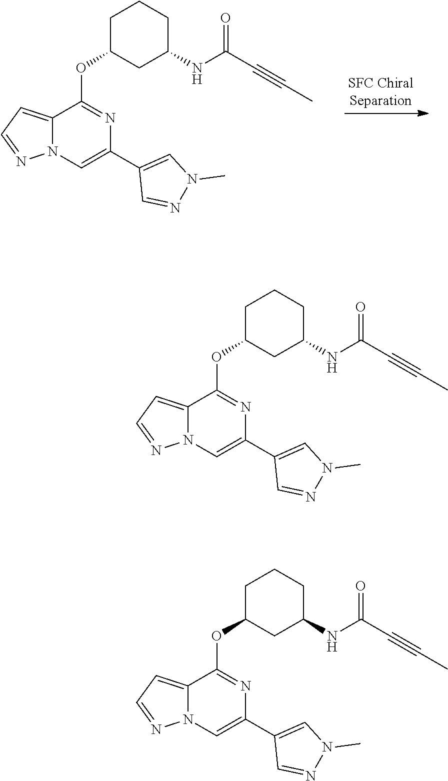 [SMILES: CC#CC(=O)N[C@H]1CCC[C@@H](Oc2nc(-c3cnn(C)c3)cn3nccc23)C1 ; CC#CC(=O)N[C@H]1CCC[C@@H](Oc2nc(-c3cnn(C)c3)cn3nccc23)C1 ; CC#CC(=O)N[C@@H]1CCC[C@H](Oc2nc(-c3cnn(C)c3)cn3nccc23)C1]

rac-N-((1R,3S)-3-((6-(1-methyl-1H-pyrazol-4-yl)pyrazolo[1,5-a]pyrazin-4-yl)oxy)cyclohexyl)but-2-ynamide (70 mg) was resolved by chiral SFC purification (CHIRALPAK IA-H 30×250 mm, 5 μm, 40% MeOH in CO₂, flow rate: 100 mL/min, ABPR 120 bar, MBPR 40 psi, column temp 40° C.) to afford the two products as a first eluting peak (Example 179), Peak 1 (23 mg, 31.2%, Rt=2.09 min, 99.88% ee) and a second eluting peak (Example 180), Peak 2 (21 mg, 28.5%, Rt=2.60 min, 96.26% ee) as off-white solids. ESI-MS (M+H)⁺: 379.1.

### Example 181: 2-chloro-N-methyl-N-((1S,3R)-3-((6-(1-methyl-1H-pyrazol-4-yl)pyrazolo[1,5-a]pyrazin-4-yl)oxy)cyclohexyl)acetamide

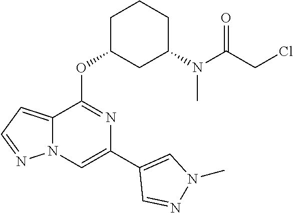 [SMILES: CN(C(=O)CCl)[C@H]1CCC[C@@H](Oc2nc(-c3cnn(C)c3)cn3nccc23)C1]

### 1. Synthesis of tert-butyl methyl((1S,3R)-3-((6-(1-methyl-1H-pyrazol-4-yl)pyrazolo[1,5-a]pyrazin-4-yl)oxy)cyclohexyl)carbamate

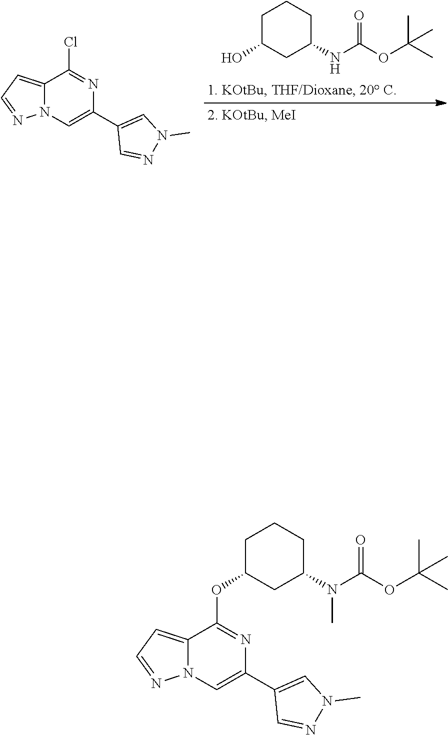 [SMILES: Cn1cc(-c2cn3nccc3c(Cl)n2)cn1 ; CC(C)(C)OC(=O)N[C@H]1CCC[C@@H](O)C1 ; CN(C(=O)OC(C)(C)C)[C@H]1CCC[C@@H](Oc2nc(-c3cnn(C)c3)cn3nccc23)C1]

KOtBu (1 M, 1.11 mL) was added to a solution of 4-chloro-6-(1-methylpyrazol-4-yl)pyrazolo[1,5-a]pyrazine (260 mg, 1.11 mmol), tert-butyl ((1S,3R)-3-hydroxycyclohexyl)carbamate (250 mg, 1.16 mmol) in THF (3 mL) and DMF (3 mL) and the mixture stirred at RT for 15 mins. To this was added Mel (474 mg, 3.34 mmol) followed by KOtBu (1 M, 2.23 mL) and stirring continued for 30 mins at RT. The reaction mixture was evaporated to dryness in vacuo and the residue partitioned between MTBE (10 mL) and NaHCO₃ (10 mL). The aqueous layer was further extracted with MTBE (3×10 mL). The combined organics were concentrated to dryness and purified by silica gel column chromatography (24 g, heptane to EtOAc) to afford tert-butyl methyl((1S,3R)-3-((6-(1-methyl-1H-pyrazol-4-yl)pyrazolo[1,5-a]pyrazin-4-yl)oxy)cyclohexyl)carbamate (393 mg, 83% yield). ESI-MS (M+H)⁺: 427.2. 1H NMR (DMSO-d6) δ: 8.74 (s, 1H), 8.16-8.31 (m, 1H), 8.01 (d, J=1.8 Hz, 2H), 6.79 (d, J=1.2 Hz, 1H), 5.20-5.40 (m, 1H), 3.89 (s, 3H), 2.71 (s, 3H), 2.11-2.31 (m, 2H), 1.86 (br s, 1H), 1.46-1.76 (m, 4H), 1.41 (s, 10H).

### 2. Synthesis of (1S,3R)—N-methyl-3-((6-(1-methyl-1H-pyrazol-4-yl)pyrazolo[1,5-a]pyrazin-4-yl)oxy)cyclohexan-1-amine hydrochloride

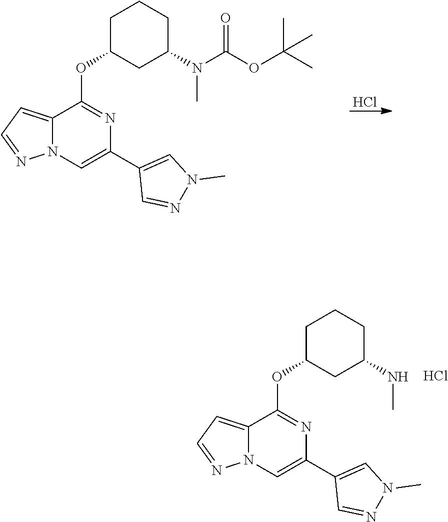 [SMILES: CN(C(=O)OC(C)(C)C)[C@H]1CCC[C@@H](Oc2nc(-c3cnn(C)c3)cn3nccc23)C1 ; Cl ; CN[C@H]1CCC[C@@H](Oc2nc(-c3cnn(C)c3)cn3nccc23)C1 ; Cl]

HCl (1.25 M in MeOH, 5 mL) was added to tert-butyl methyl((1S,3R)-3-((6-(1-methyl-1H-pyrazol-4-yl)pyrazolo[1,5-a]pyrazin-4-yl)oxy)cyclohexyl)carbamate (393 mg, 921.43 umol) and the mixture stirred at 45° C. for 3 h. The reaction mixture was concentrated to dryness, diluted with 5 mL EtOAc and re-evaporated to give (1S,3R)—N-methyl-3-((6-(1-methyl-1H-pyrazol-4-yl)pyrazolo[1,5-a]pyrazin-4-yl)oxy)cyclohexan-1-amine hydrochloride as a white solid (425 mg, 127% yield). ESI-MS (M+H)+: 327.1. 1H NMR (DMSO-d6) δ: 9.11 (br s, 2H), 8.76 (s, 1H), 8.28 (s, 1H), 8.04 (s, 1H), 8.02 (d, J=2.4 Hz, 1H), 6.84 (d, J=2.4 Hz, 1H), 5.27-5.37 (m, 1H), 3.85-3.95 (m, 3H), 3.20-3.34 (m, 1H), 2.69 (br d, J=11.6 Hz, 1H), 2.55 (t, J=5.5 Hz, 3H), 2.21 (br d, J=10.4 Hz, 1H), 2.09 (br d, J=11.6 Hz, 1H), 1.87-1.96 (m, 1H), 1.56-1.67 (m, 1H), 1.29-1.56 (m, 3H).

### 3. Synthesis of 2-chloro-N-methyl-N-((1S,3R)-3-((6-(1-methyl-1H-pyrazol-4-yl)pyrazolo[1,5-a]pyrazin-4-yl)oxy)cyclohexyl)acetamide

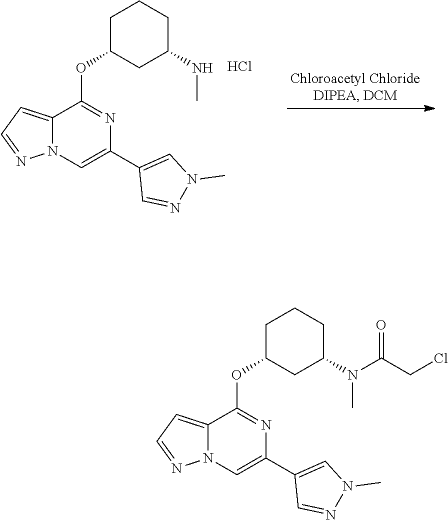 [SMILES: CN[C@H]1CCC[C@@H](Oc2nc(-c3cnn(C)c3)cn3nccc23)C1 ; Cl ; CN(C(=O)CCl)[C@H]1CCC[C@@H](Oc2nc(-c3cnn(C)c3)cn3nccc23)C1]

To a solution of (1S,3R)—N-methyl-3-((6-(1-methyl-1H-pyrazol-4-yl)pyrazolo[1,5-a]pyrazin-4-yl)oxy)cyclohexan-1-amine hydrochloride (50 mg, 0.138 mmol) in DCM (3 mL) was added DIPEA (92.6 mg, 717 μmol) and the solution cooled to −20° C. Chloroacetyl chloride (16.2 mg, 143 μmol) was added and the reaction stirred for 10 mins. The reaction was purified via chromatography (heptane to 3:1 EtOAc:EtOH) to afford 40 mg (71% yield) of 2-chloro-N-methyl-N-((1S,3R)-3-((6-(1-methyl-1H-pyrazol-4-yl)pyrazolo[1,5-a]pyrazin-4-yl)oxy)cyclohexyl)acetamide. LCMS m/z=403.1(M+H)⁺. ¹H NMR (500 MHz, DMSO-d₆) δ ppm 1.39-1.49 (m, 1H) 1.39-1.49 (m, 1H) 1.50-1.64 (m, 3H) 1.71 (q, J=11.39 Hz, 1H) 1.81-1.92 (m, 1H) 2.14-2.32 (m, 2H) 2.74-2.94 (m, 2H) 3.86-3.92 (m, 3H) 4.31-4.56 (m, 3H) 5.30-5.46 (m, 1H) 6.78 (d, J=2.44 Hz, 1H) 7.98-8.06 (m, 2H) 8.18-8.26 (m, 1H) 8.71-8.78 (m, 1H).

### Example 182: N-methyl-N-(3-((6-(1-methyl-1H-pyrazol-4-yl)pyrazolo[1,5-a]pyrazin-4-yl)oxy)propyl)acrylamide

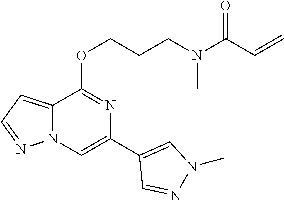 [SMILES: C=CC(=O)N(C)CCCOc1nc(-c2cnn(C)c2)cn2nccc12]

### 1. Synthesis of N-methyl-3-((6-(1-methyl-1H-pyrazol-4-yl)pyrazolo[1,5-a]pyrazin-4-yl)oxy)propan-1-amine hydrochloride

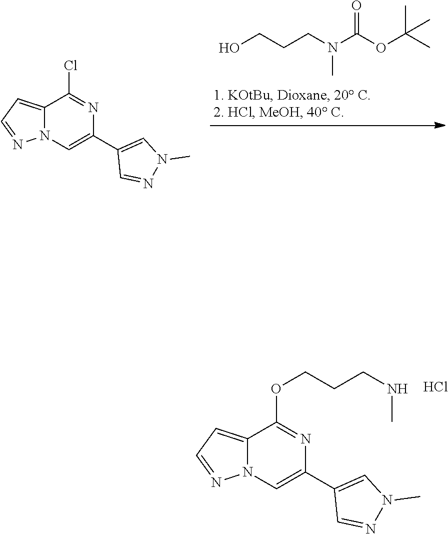 [SMILES: Cn1cc(-c2cn3nccc3c(Cl)n2)cn1 ; CN(CCCO)C(=O)OC(C)(C)C ; CNCCCOc1nc(-c2cnn(C)c2)cn2nccc12 ; Cl]

To a solution of 4-chloro-6-(1-methylpyrazol-4-yl)pyrazolo[1,5-a]pyrazine (Intermediate A, 100 mg, 428 μmol) and tert-butyl N-(3-hydroxypropyl)-N-methyl-carbamate (90 mg, 476 μmol) in THF (3 mL) was added KOtBu (1 M, 500 uL) and the reaction stirred at RT for 5 mins. The reaction mixture was concentrated to dryness and the crude intermediate was dissolved in HCl (1.25 M in MeOH, 2 mL) and stirred at 40° C. for 16 h. The mixture was concentrated to dryness, diluted with EtOAc (10 mL) and concentrated to give N-methyl-3-((6-(1-methyl-1H-pyrazol-4-yl)pyrazolo[1,5-a]pyrazin-4-yl)oxy)propan-1-amine hydrochloride as a white solid. ESI-MS (M+H)⁺: 287.0.

### 2. Synthesis of N-methyl-N-(3-((6-(1-methyl-1H-pyrazol-4-yl)pyrazolo[1,5-a]pyrazin-4-yl)oxy)propyl)acrylamide

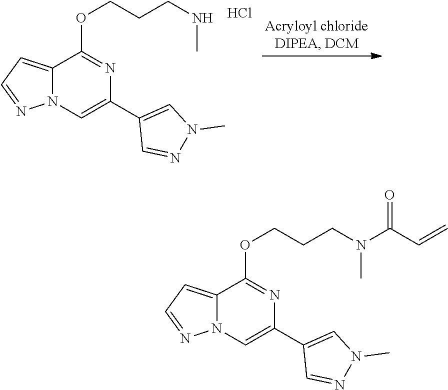 [SMILES: CNCCCOc1nc(-c2cnn(C)c2)cn2nccc12 ; Cl ; C=CC(=O)N(C)CCCOc1nc(-c2cnn(C)c2)cn2nccc12]

N-Methyl-3-((6-(1-methyl-1H-pyrazol-4-yl)pyrazolo[1,5-a]pyrazin-4-yl)oxy)propan-1-amine hydrochloride (65 mg, 200 μmol) was treated with THF (3 mL) and TEA (101 mg, 1.0 mmol) and the solution cooled to −20° C. Acryloyl chloride (18 mg, 200 μmol) was added and the reaction stirred for 5 mins. The reaction mixture was diluted with NaHCO₃ (3 mL) and extracted with EtOAc (3×3 mL). The combined organic layers were concentrated to dryness and the crude product purified by prep-HPLC using a Waters Sunfire Prep C18, 5 μm, 19 mm×100 mm column with mobile phase H₂O (A) and MeCN (B) and a gradient of 5-60% B (0.2% NH₄HCO₃ final v/v % modifier) and a flow rate at 30 mL/min, to give N-methyl-N-(3-((6-(1-methyl-1H-pyrazol-4-yl)pyrazolo[1,5-a]pyrazin-4-yl)oxy)propyl)acrylamide (22 mg, 32% yield). ESI-MS (M+H)⁺: 363.1. ¹H NMR (DMSO-d₆) δ: 8.76 (d, J=2.4 Hz, 1H), 8.19 (d, J=4.9 Hz, 1H), 8.02-8.09 (m, 1H), 8.01 (s, 1H), 6.69-6.94 (m, 2H), 6.08 (d, J=2.4 Hz, 1H), 5.48-5.71 (m, 1H), 4.50-4.63 (m, 2H), 3.89 (s, 3H), 3.52-3.70 (m, 2H), 2.91-3.12 (m, 3H), 2.03-2.15 (m, 2H).

### Example 183: N-methyl-N-(3-((6-(1-methyl-1H-pyrazol-4-yl)pyrazolo[1,5-a]pyrazin-4-yl)oxy)ethyl)acrylamide

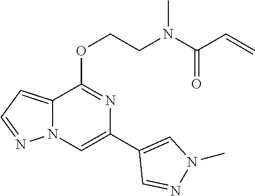 [SMILES: C=CC(=O)N(C)CCOc1nc(-c2cnn(C)c2)cn2nccc12]

### 1. Synthesis of N-methyl-2-((6-(1-methyl-1H-pyrazol-4-yl)pyrazolo[1,5-a]pyrazin-4-yl)oxy)ethan-1-amine hydrochloride

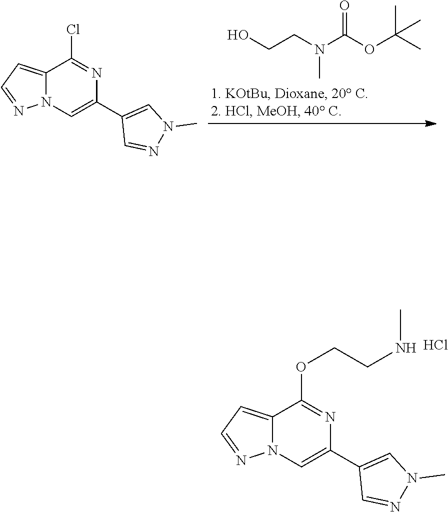 [SMILES: Cn1cc(-c2cn3nccc3c(Cl)n2)cn1 ; CN(CCO)C(=O)OC(C)(C)C ; CNCCOc1nc(-c2cnn(C)c2)cn2nccc12 ; Cl]

To a solution of 4-chloro-6-(1-methylpyrazol-4-yl)pyrazolo[1,5-a]pyrazine (Intermediate A, 100 mg, 428 μmol), tert-butyl N-(2-hydroxyethyl)-N-methyl-carbamate (80 mg, 457 μmol) and THF (3 mL) was added KOtBu (1 M, 500 uL) and the reaction stirred at RT for 5 mins. The mixture was concentrated to dryness, HCl (1.25 M, 2 mL) was added and the solution stirred at 40° C. for 16 h. The mixture was concentrated to dryness, diluted with EtOAc (10 mL) and concentrated to give N-methyl-2-((6-(1-methyl-1H-pyrazol-4-yl)pyrazolo[1,5-a]pyrazin-4-yl)oxy)ethan-1-amine hydrochloride as a white solid. ESI-MS (M+H)⁺: 273.0.

### 2. Synthesis of N-methyl-N-(2-((6-(1-methyl-1H-pyrazol-4-yl)pyrazolo[1,5-a]pyrazin-4-yl)oxy)ethyl)acrylamide

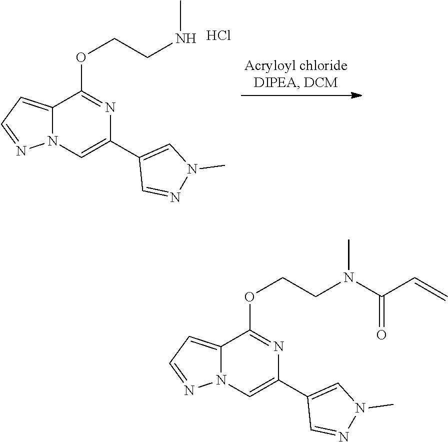 [SMILES: CNCCOc1nc(-c2cnn(C)c2)cn2nccc12 ; Cl ; C=CC(=O)N(C)CCOc1nc(-c2cnn(C)c2)cn2nccc12]

N-Methyl-N-(2-((6-(1-methyl-1H-pyrazol-4-yl)pyrazolo[1,5-a]pyrazin-4-yl)oxy)ethyl)acrylamide was obtained, 60 mg, 92% yield, from N-methyl-2-((6-(1-methyl-1H-pyrazol-4-yl)pyrazolo[1,5-a]pyrazin-4-yl)oxy)ethan-1-amine hydrochloride and acryloyl chloride following the procedure described in step 2, Example 182. ESI-MS (M+H)⁺: 349.0. ¹H NMR (DMSO-d₆) δ: 8.77 (s, 1H), 8.22 (d, J=9.8 Hz, 1H), 7.98-8.05 (m, 2H), 6.67-6.94 (m, 2H), 6.04-6.15 (m, 1H), 5.57-5.72 (m, 1H), 4.70 (q, J=5.5 Hz, 2H), 3.77-3.98 (m, 5H), 2.98 (s, 3H).

### Example 184: N-methyl-N-(2-methyl-4-((6-(1-methyl-1H-pyrazol-4-yl)pyrazolo[1,5-a]pyrazin-4-yl)oxy)butyl)acrylamide

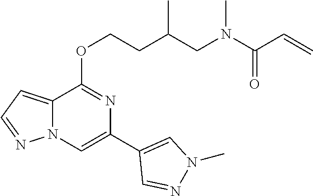 [SMILES: C=CC(=O)N(C)CC(C)CCOc1nc(-c2cnn(C)c2)cn2nccc12]

### 1. Synthesis of N,2-dimethyl-4-((6-(1-methyl-1H-pyrazol-4-yl)pyrazolo[1,5-a]pyrazin-4-yl)oxy)butan-1-amine trifluoroacetate

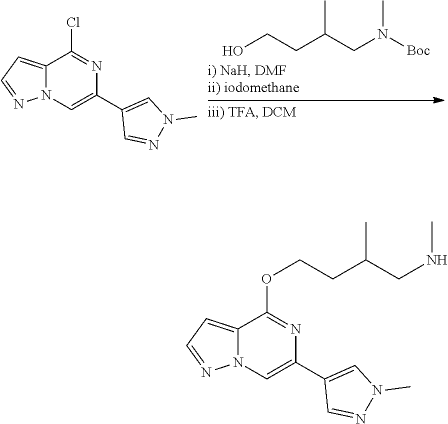 [SMILES: Cn1cc(-c2cn3nccc3c(Cl)n2)cn1 ; CC(CCO)CN(C)C(=O)OC(C)(C)C ; CNCC(C)CCOc1nc(-c2cnn(C)c2)cn2nccc12]

To a solution of 4-chloro-6-(1-methyl-1H-pyrazol-4-yl)pyrazolo[1,5-a]pyrazine (50 mg, 214 μmol) and tert-butyl (4-hydroxy-2-methylbutyl)(methyl)carbamate (48 mg, 235 μmol) in DMF (2 mL) was added sodium hydride (26 mg, 642 μmol) at RT and the mixture stirred for 30 mins. Iodomethane (20 μL, 321 μmol) was added and the reaction stirred for a further 30 mins. EtOAc (5 mL) were added to the vigorously stirred reaction mixture and the resulting organic phase was washed with sat. aq. NaHCO₃ (5 mL), water (5 mL), and brine (5 mL). The organic phase was separated, dried over Na₂SO₄, filtered, and concentrated under reduced pressure to yield a pale orange residue. This was dissolved in DCM (3 mL) and TFA (1 mL) was added and the solution stirred at RT overnight. The reaction was evaporated under reduced pressure to give N,2-dimethyl-4-((6-(1-methyl-1H-pyrazol-4-yl)pyrazolo[1,5-a]pyrazin-4-yl)oxy)butan-1-amine trifluoroacetate. The crude material was split in half and one half was used in the next step without further purification. LCMS m/z=315.0 (M+H)⁺.

### 2. Synthesis of N-methyl-N-(2-methyl-4-((6-(1-methyl-1H-pyrazol-4-yl)pyrazolo[1,5-a]pyrazin-4-yl)oxy)butyl)acrylamide

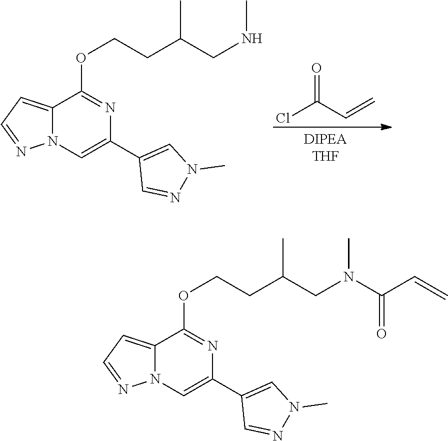

N-methyl-N-(2-methyl-4-((6-(1-methyl-1H-pyrazol-4-yl)pyrazolo[1,5-a]pyrazin-4-yl)oxy)butyl)acrylamide was synthesized in the same way as Example 27 but starting from crude N,2-dimethyl-4-((6-(1-methyl-1H-pyrazol-4-yl)pyrazolo[1,5-a]pyrazin-4-yl)oxy)butan-1-amine trifluoroacetate (33 mg, 105 μmol). The material was purified by column chromatography (SiO₂, 0-100% EtOH:EtOAc (2% NH₄OH)_{1:3} in heptane) to give the product (13 mg, 33% yield) as a white solid. LCMS m/z=369.2 (M+H)⁺. ¹H NMR (500 MHz, MeOH-d₄) δ ppm 8.35-8.42 (m, 1H), 8.06 (s, 1H), 7.89-7.97 (m, 2H), 6.68-6.81 (m, 2H), 6.14-6.25 (m, 1H), 5.63-5.75 (m, 1H), 4.57-4.72 (m, 2H), 3.93 (d, J=1.22 Hz, 3H), 3.46-3.54 (m, 1H), 3.32-3.41 (m, 1H), 2.94-3.17 (m, 3H), 2.08-2.23 (m, 1H), 1.89-2.03 (m, 1H), 1.62-1.76 (m, 1H), 1.02 (dd, J=1.83, 6.71 Hz, 3H).

### Example 185: N-methyl-N-((trans)-3-((6-(1-methyl-1H-pyrazol-4-yl)pyrazolo[1,5-a]pyrazin-4-yl)oxy)cyclobutyl)acrylamide

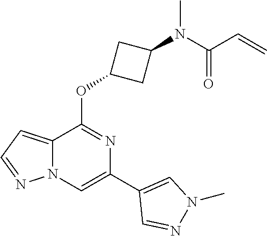 [SMILES: C=CC(=O)N(C)[C@H]1C[C@H](Oc2nc(-c3cnn(C)c3)cn3nccc23)C1]

### 1. Synthesis of (trans)-N-methyl-3-((6-(1-methyl-1H-pyrazol-4-yl)pyrazolo[1,5-a]pyrazin-4-yl)oxy)cyclobutan-1-amine

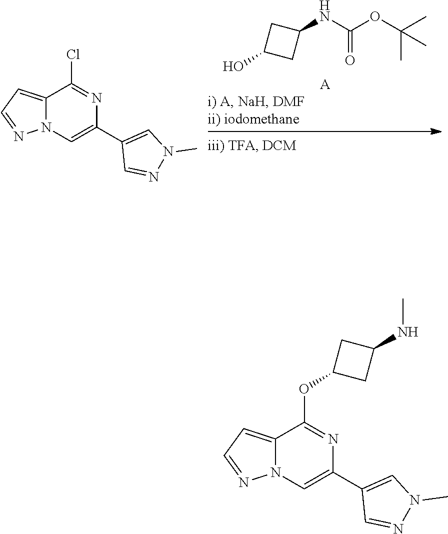

(trans)-N-methyl-3-((6-(1-methyl-1H-pyrazol-4-yl)pyrazolo[1,5-a]pyrazin-4-yl)oxy)cyclobutan-1-amine was synthesized in the same way as step 1, Example 184 but starting from tert-butyl ((trans)-3-hydroxycyclobutyl)carbamate. The crude material was split in half and used in the next step without further purification (assuming 100% yield). LCMS m/z=299.0 (M+H)⁺.

### 2. Synthesis of N-methyl-N-((trans)-3-((6-(1-methyl-1H-pyrazol-4-yl)pyrazolo[1,5-a]pyrazin-4-yl)oxy)cyclobutyl)acrylamide

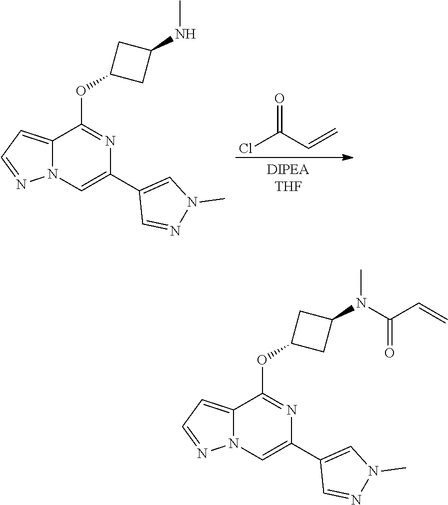

N-Methyl-N-((trans)-3-((6-(1-methyl-1H-pyrazol-4-yl)pyrazolo[1,5-a]pyrazin-4-yl)oxy)cyclobutyl)acrylamide (37 mg, 72%) was synthesized from (trans)-N-methyl-3-((6-(1-methyl-1H-pyrazol-4-yl)pyrazolo[1,5-a]pyrazin-4-yl)oxy)cyclobutan-1-amine (31 mg, 0.104 mmol) using an analogous method to that described for Example 27. Purification was by column chromatography (24 g SiO₂, 0-100% EtOH:EtOAc (2% NH₄OH)1:3 in heptane) LCMS m/z=375.1 (M+Na)⁺. ¹H NMR (500 MHz, MeOH-d₄) δ ppm 8.35 (br s, 1H), 7.99 (s, 1H), 7.85-7.94 (m, 2H), 6.80 (d, J=1.83 Hz, 1H), 6.72 (br s, 1H), 6.08-6.28 (m, 1H), 5.73 (br s, 1H), 5.43-5.54 (m, 1H), 4.93-5.29 (m, 1H), 3.91 (s, 3H), 3.04-3.21 (m, 3H), 2.74-2.95 (m, 2H), 2.60 (br s, 2H).

### Example 186 N-((1s,3s)-3-methyl-3-((6-(2-methylthiazol-5-yl)pyrazolo[1,5-a]pyrazin-4-yl)oxy)cyclobutyl)acrylamide

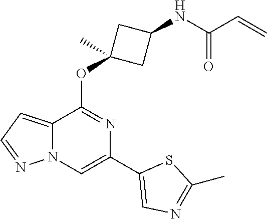 [SMILES: C=CC(=O)N[C@H]1C[C@@](C)(Oc2nc(-c3cnc(C)s3)cn3nccc23)C1]

### 1. Synthesis of tert-butyl ((1s,3s)-3-((6-chloropyrazolo[1,5-a]pyrazin-4-yl)oxy)-3-methylcyclobutyl)carbamate

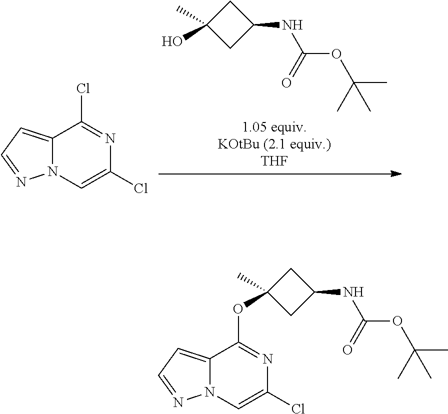 [SMILES: Clc1cn2nccc2c(Cl)n1 ; CC(C)(C)OC(=O)N[C@H]1C[C@@](C)(O)C1 ; CC(C)(C)OC(=O)N[C@H]1C[C@@](C)(Oc2nc(Cl)cn3nccc23)C1]

To a solution of tert-butyl N-(3-hydroxy-3-methyl-cyclobutyl)carbamate (5 g, 24.84 mmol) in THF (150 mL) was added KOtBu (5.58 g, 49.7 mmol) in batches at RT. Upon stirring at rt for 15 min a thick off-white suspension formed. The reaction mixture was cooled in an ice water bath and a solution of 4,6-dichloropyrazolo[1,5-a]pyrazine (4.45 g, 23.66 mmol) in THF (50 mL) was added dropwise over 30 min. After 30 min, the reaction mixture was diluted with EtOAc (200 mL) and the organic phase was extracted with water (2×100 mL) and brine (100 mL). The organic phase was dried over Na₂SO₄, filtered and concentrated. The crude residue was purified by column chromatography (0-100% EtOH:EtOAc (2% NH₄OH)_{1:3} in heptane) to give tert-butyl ((1s,3s)-3-((6-chloropyrazolo[1,5-a]pyrazin-4-yl)oxy)-3-methylcyclobutyl)carbamate (5.45 g, 65% yield) as a beige solid. LCMS m/z=353.1 (M+H)⁺. ¹H NMR (500 MHz, MeOH-d₄) δ ppm 8.28 (s, 1H), 7.95 (d, J=2.44 Hz, 1H), 6.83 (d, J=2.44 Hz, 1H), 3.86 (br t, J=7.94 Hz, 1H), 2.75-2.84 (m, 2H), 2.35-2.45 (m, 2H), 1.74 (s, 3H), 1.43 (s, 9H).

### 2. Synthesis of tert-butyl ((1s,3s)-3-methyl-3-((6-(2-methylthiazol-5-yl)pyrazolo[1,5-a]pyrazin-4-yl)oxy)cyclobutyl)carbamate

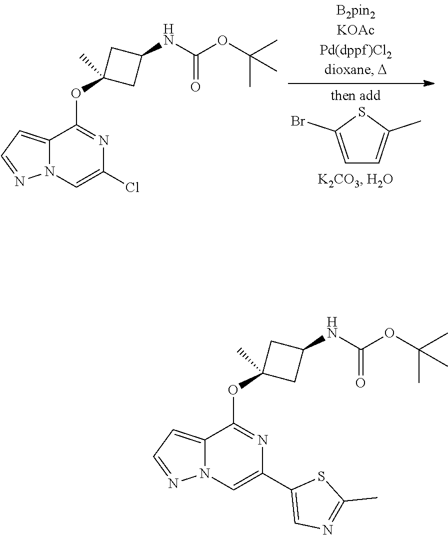

To a vial was added tert-butyl ((1s,3s)-3-((6-chloropyrazolo[1,5-a]pyrazin-4-yl)oxy)-3-methylcyclobutyl)carbamate (160 mg, 0.45 mmol), bis(pinacolato)diboron (150 mg, 0.59 mmol), potassium acetate (140 mg, 1.4 mmol), and Pd(dppf)Cl₂ (20.0 mg, 31 μmol) followed by dioxane (2.0 mL). The mixture was sparged with N₂ for 30 mins, then sealed and warmed to 90° C. The reaction was left to stir for 30 mins at this temperature and then heated to 100° C. for 6 h. K₂CO₃ (125 mg, 0.91 mmol) and 5-bromo-2-methyl-thiazole (107 mg, 0.6 mmol) followed by water (1.0 mL) were added and the vial was stirred at 100° C. for 45 mins. The reaction was removed from heating, cooled to RT, diluted with water (10 mL) and extracted with EtOAc (3×10 mL). The combined organic layers were concentrated to dryness, then purified by silica gel chromatography (24 g, heptane to EtOAc) to give tert-butyl ((1s,3s)-3-methyl-3-((6-(2-methylthiazol-5-yl)pyrazolo[1,5-a]pyrazin-4-yl)oxy)cyclobutyl)carbamate as a white solid (105 mg, 56% yield). LCMS: m/z=438.1 (M+Na)⁺.

### 3. Synthesis of (1s,3s)-3-methyl-3-((6-(2-methylthiazol-5-yl)pyrazolo[1,5-a]pyrazin-4-yl)oxy)cyclobutan-1-amine hydrochloride

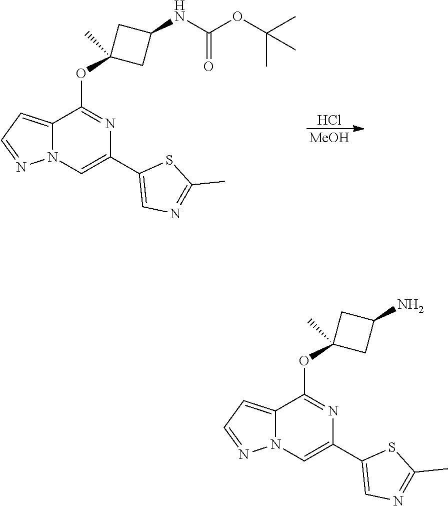 [SMILES: "MeOH" ; Cc1ncc(-c2cn3nccc3c(O[C@]3(C)C[C@H](NC(=O)OC(C)(C)C)C3)n2)s1 ; Cl ; Cc1ncc(-c2cn3nccc3c(O[C@]3(C)C[C@H](N)C3)n2)s1]

(1s,3s)-3-Methyl-3-((6-(2-methylthiazol-5-yl)pyrazolo[1,5-a]pyrazin-4-yl)oxy)cyclobutan-1-amine hydrochloride was obtained as an off-white solid, from tert-butyl ((1s,3s)-3-methyl-3-((6-(2-methylthiazol-5-yl)pyrazolo[1,5-a]pyrazin-4-yl)oxy)cyclobutyl), following the procedure described in step 2, Example 149. LCMS: m/z=316.0 (M+H)⁺.

### 4. Synthesis of N—((1s,3s)-3-methyl-3-((6-(2-methylthiazol-5-yl)pyrazolo[1,5-a]pyrazin-4-yl)oxy)cyclobutyl)acrylamide

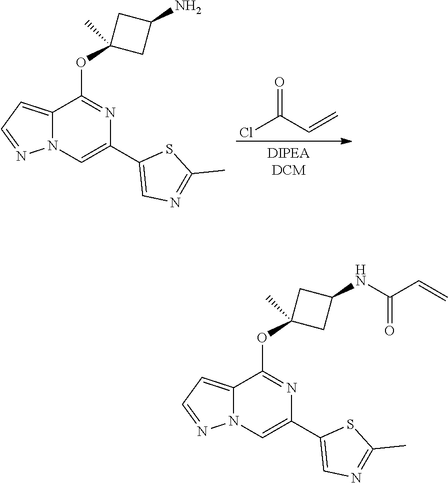

To a vial containing (1s,3s)-3-methyl-3-((6-(2-methylthiazol-5-yl)pyrazolo[1,5-a]pyrazin-4-yl)oxy)cyclobutan-1-amine hydrochloride (90 mg, 0.256 mmol) was added DCM (3.0 mL) and DIPEA (150 μL, 0.861 mmol), the solution cooled to −20° C. Acryloyl chloride (20.8 μL, 0.256 mmol) was added and the reaction was stirred for 10 mins. The mixture was purified directly by silica gel chromatography (heptane to EtOAc) to give N—((1s,3s)-3-methyl-3-((6-(2-methylthiazol-5-yl)pyrazolo[1,5-a]pyrazin-4-yl)oxy)cyclobutyl)acrylamide as a light yellow solid, (70 mg, 73% yield over 2 steps). LCMS: m/z=370.1 (M+H)⁺. ¹H NMR (500 MHz, DMSO-d₆): δ ppm=1.75 (s, 3H) 2.39-2.46 (m, 2H) 2.67 (s, 3H) 2.82 (ddd, J=9.92, 7.48, 2.75 Hz, 2H) 4.18 (sxt, J=7.81 Hz, 1H) 5.52-5.64 (m, 1H) 6.03-6.19 (m, 2H) 6.90 (d, J=3.05 Hz, 1H) 8.10 (d, J=2.44 Hz, 1H) 8.26 (s, 1H) 8.43 (br d, J=7.94 Hz, 1H) 9.08 (s, 1H).

Examples 187 & 188: (R)-1-(3-fluoro-3-(((6-(1-methyl-1H-pyrazol-4-yl)pyrazolo[1,5-a]pyrazin-4-yl)oxy)methyl)piperidin-1-yl)prop-2-en-1-one and (S)-1-(3-fluoro-3-(((6-(1-methyl-1H-pyrazol-4-yl)pyrazolo[1,5-a]pyrazin-4-yl)oxy)methyl)piperidin-1-yl)prop-2-en-1-one

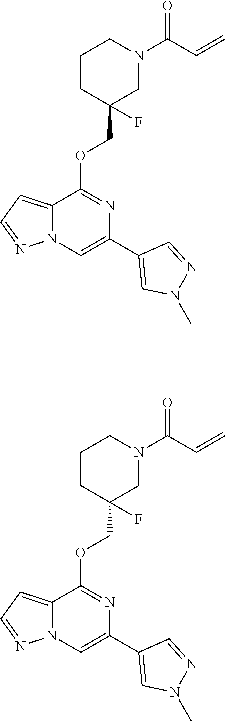 [SMILES: C=CC(=O)N1CCC[C@](F)(COc2nc(-c3cnn(C)c3)cn3nccc23)C1 ; C=CC(=O)N1CCC[C@@](F)(COc2nc(-c3cnn(C)c3)cn3nccc23)C1]

### 1. Synthesis of tert-butyl 3-fluoro-3-(((6-(1-methyl-1H-pyrazol-4-yl)pyrazolo[1,5-a]pyrazin-4-yl)oxy)methyl)piperidine-1-carboxylate

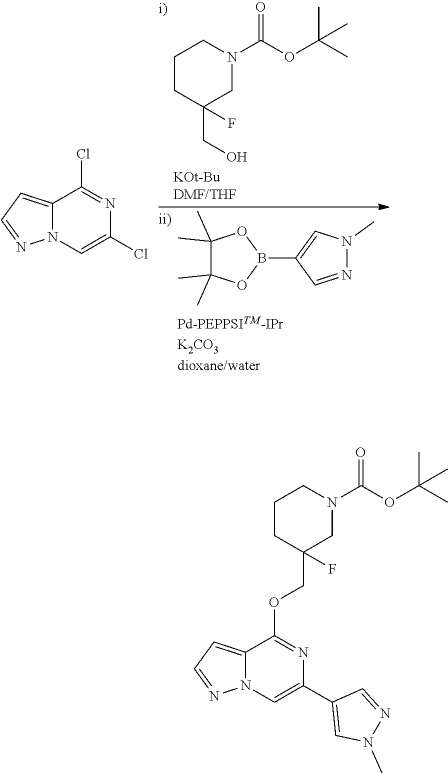

i) To a vial containing tert-butyl 3-fluoro-3-(hydroxymethyl)piperidine-1-carboxylate (140 mg, 0.60 mmol) was added 1.0 mL of a solution of 4,6-dichloropyrazolo[1,5-a]pyrazine in DMF (0.53 M, 100 mg, 0.53 mmol) then a solution of KOt-Bu (1.0 M in THF, 700 μL) and the reaction allowed to stir for 30 mins at RT.

A stock solution of 1-methyl-4-(4,4,5,5-tetramethyl-1,3,2-dioxaborolan-2-yl)pyrazole (1.6 g, 7.97 mmol) and Pd-PEPPSI™-IPr (100 mg, 0.16 mmol) in dioxane (20 mL) was prepared, followed by preparation of a stock solution of K₂CO₃ (775 mg, 10.6 mmol) in water (10 mL).

ii) To the vial containing the product from part i) was added 2.0 mL of the dioxane solution and 1.0 mL of the solution in water. The vial was capped and stirred overnight at 100° C. The reaction mixture was diluted with H₂O (15 mL) and extracted with EtOAc (3×10 mL). The combined organic layers were concentrated to dryness and purified by silica gel chromatography (heptane to EtOAc) to provide tert-butyl 3-fluoro-3-(((6-(1-methyl-1H-pyrazol-4-yl)pyrazolo[1,5-a]pyrazin-4-yl)oxy)methyl)piperidine-1-carboxylate (160 mg, 70% yield). LC-MS: m/z=431.1 (M+H)⁺.

### 2. Synthesis of 4-((3-fluoropiperidin-3-yl)methoxy)-6-(1-methyl-1H-pyrazol-4-yl)pyrazolo[1,5-a]pyrazine hydrochloride

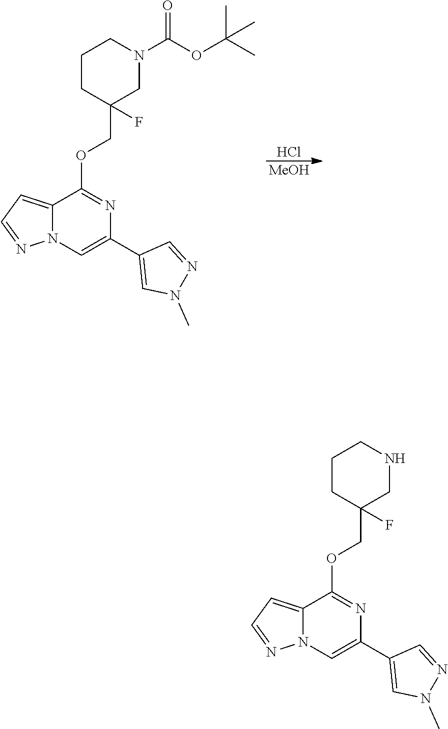 [SMILES: "MeOH" ; Cn1cc(-c2cn3nccc3c(OCC3(F)CCCN(C(=O)OC(C)(C)C)C3)n2)cn1 ; Cl ; Cn1cc(-c2cn3nccc3c(OCC3(F)CCCNC3)n2)cn1]

4-((3-Fluoropiperidin-3-yl)methoxy)-6-(1-methyl-1H-pyrazol-4-yl)pyrazolo[1,5-a]pyrazine hydrochloride was obtained, 140 mg, quantitatively, from tert-butyl 3-fluoro-3-(((6-(1-methyl-1H-pyrazol-4-yl)pyrazolo[1,5-a]pyrazin-4-yl)oxy)methyl)piperidine-1-carboxylate following the procedure described in step 2, Example 149. LCMS: m/z=331.1 (M+H)⁺.

### 3. Synthesis of (R)-1-(3-fluoro-3-(((6-(1-methyl-1H-pyrazol-4-yl)pyrazolo[1,5-a]pyrazin-4-yl)oxy)methyl)piperidin-1-yl)prop-2-en-1-one and (S)-1-(3-fluoro-3-(((6-(1-methyl-1H-pyrazol-4-yl)pyrazolo[1,5-a]pyrazin-4-yl)oxy)methyl)piperidin-1-yl)prop-2-en-1-one

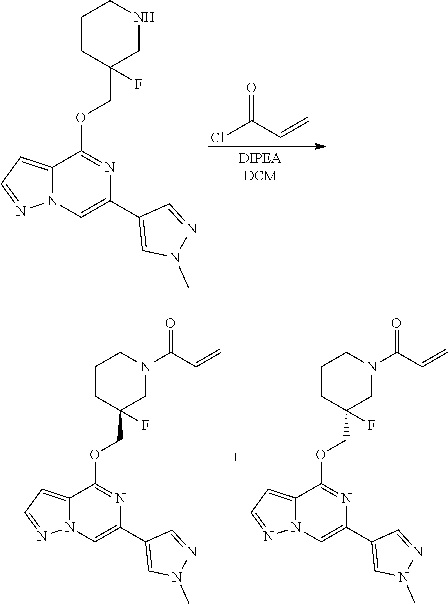

To a vial containing 4-((3-fluoropiperidin-3-yl)methoxy)-6-(1-methyl-1H-pyrazol-4-yl)pyrazolo[1,5-a]pyrazine hydrochloride (140 mg, 0.382 mmol) was added DCM (5 mL) and DIPEA (332 μL, 1.91 mmol) and the mixture was stirred for 5 mins. The reaction mixture was cooled in a dry ice/MeCN bath, acryloyl chloride (31.0 μL, 0.382 mmol) was added and the reaction stirred for 10 mins. The reaction mixture was purified directly by silica gel chromatography (heptane to 3:1 EtOAc:EtOH) to give 1-(3-fluoro-3-(((6-(1-methyl-1H-pyrazol-4-yl)pyrazolo[1,5-a]pyrazin-4-yl)oxy)methyl)piperidin-1-yl)prop-2-en-1-one (100 mg, 68% yield over 2 steps). LCMS: m/z=385.1 (M+H)⁺.

The racemic product was dissolved in i-PrOH with 0.25% NEt₃ and separated by chiral SFC (3.0×25.0 cm RegisPack column; 30% i-PrOH with 0.25% TFA in CO₂); flow rate=100 mL/min, ABPR 100 bar, MBPR 40 psi, column temp. 25° C.), to provide the first eluting enantiomer (22.0 mg, 95.7% ee, Rf=2.65 min) (Example 187) and the second eluting enantiomer (25.0 mg, 95.0% ee, Rf=2.81 min) (Example 188).

### Example 189: 1-(4-methyl-4-(((6-(1-methyl-1H-pyrazol-4-yl)pyrazolo[1,5-a]pyrazin-4-yl)oxy)methyl)piperidin-1-yl)prop-2-en-1-one

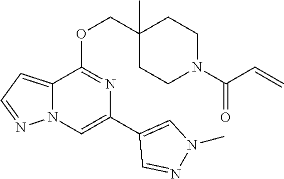 [SMILES: C=CC(=O)N1CCC(C)(COc2nc(-c3cnn(C)c3)cn3nccc23)CC1]

### 1. Synthesis of 1-(4-methyl-4-(((6-(1-methyl-1H-pyrazol-4-yl)pyrazolo[1,5-a]pyrazin-4-yl)oxy)methyl)piperidin-1-yl)prop-2-en-1-one

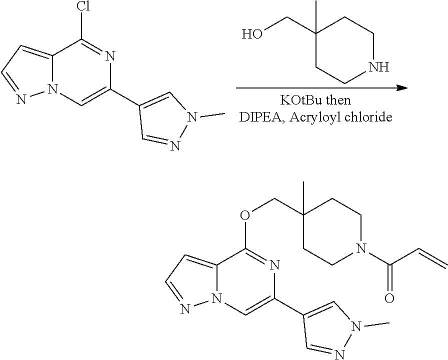

To a vial was added 4-chloro-6-(1-methylpyrazol-4-yl)pyrazolo[1,5-a]pyrazine (Intermediate A, 150 mg, 0.642 mmol), (4-methyl-4-piperidyl)methanol (90 mg, 0.697 mmol) and dioxane (3 mL) and the solution stirred for 5 mins. To this mixture was added KOtBu (1 M in THF, 1 mL) and stirring continued for 15 mins. Additional KOtBu (1 M in THF, 1 mL) was added and the mixture stirred for a further 15 mins. The crude reaction was concentrated to dryness, the crude was diluted with DCM (5 mL) and treated with DIPEA (185.50 mg, 1.44 mmol). This mixture was cooled to −20° C. and acryloyl chloride (75 mg, 0.829 mmol) was added. After 10 mins, the reaction was purified by column chromatography (heptane to 3:1 EtOAc:EtOH) to give 1-(4-methyl-4-(((6-(1-methyl-1H-pyrazol-4-yl)pyrazolo[1,5-a]pyrazin-4-yl)oxy)methyl)piperidin-1-yl)prop-2-en-1-one as a tan foam solid (100 mg, 39% yield). ESI-MS (M+H)⁺: 381.2. ¹H NMR (400 MHz, DMSO-d₆) δ ppm 8.76 (d, J=1.00 Hz, 1H), 8.23 (s, 1H), 7.99-8.05 (m, 2H), 6.87 (dd, J=2.26, 0.75 Hz, 1H), 6.83 (dd, J=16.82, 10.54 Hz, 1H), 6.10 (dd, J=16.82, 2.51 Hz, 1H), 5.66 (dd, J=10.54, 2.51 Hz, 1H), 4.41 (s, 2H), 3.95-3.87 (m, 4H), 3.73-3.83 (m, 1H), 3.42-3.53 (m, 1H), 3.27-3.32 (m, 1H), 1.54-1.69 (m, 2H), 1.42-1.53 (m, 2H), 1.16 (s, 3H).

### Examples 190 & 191: 1-((1R,5R,6S)-6-methyl-6-((6-(1-methyl-1H-pyrazol-4-yl)pyrazolo[1,5-a]pyrazin-4-yl)oxy)-2-azabicyclo[3.2.0]heptan-2-yl)prop-2-en-1-one 1-((1S,5S,6R)-6-methyl-6-((6-(1-methyl-1H-pyrazol-4-yl)pyrazolo[1,5-a]pyrazin-4-yl)oxy)-2-azabicyclo[3.2.0]heptan-2-yl)prop-2-en-1-one

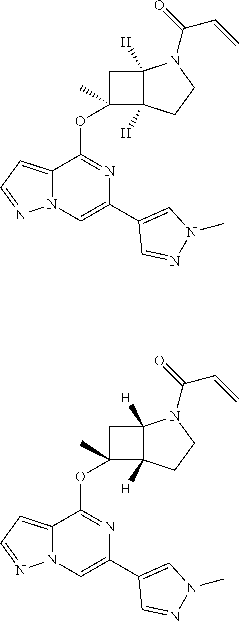 [SMILES: C=CC(=O)N1CC[C@@H]2[C@H]1C[C@]2(C)Oc1nc(-c2cnn(C)c2)cn2nccc12 ; C=CC(=O)N1CC[C@H]2[C@@H]1C[C@@]2(C)Oc1nc(-c2cnn(C)c2)cn2nccc12]

### 1. Synthesis of rac-tert-butyl (1R,5R,6S)-6-hydroxy-6-methyl-2-azabicyclo[3.2.0]heptane-2-carboxylate

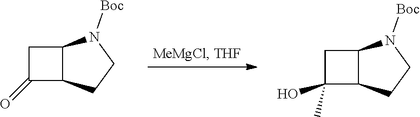 [SMILES: CC(C)(C)OC(=O)N1CC[C@H]2C(=O)C[C@H]21 ; CC(C)(C)OC(=O)N1CC[C@@H]2[C@H]1C[C@]2(C)O]

To a round bottom flask was added THF (150 mL) and methyl magnesium chloride (3 M in THF, 75 mL) which was cooled to −70° C., then a solution of rac-tert-butyl (1R,5R)-6-oxo-2-azabicyclo[3.2.0]heptane-2-carboxylate (32.5 g, 153.8 mmol) in THF (100 mL) was added dropwise over 1 h. The reaction was allowed to stir on dry ice/acetone bath for 15 mins, then allowed to warm to RT slowly. The reaction was cooled to 0° C., satd. aqueous NH₄Cl (50 mL) was added dropwise, then the mixture diluted with water (400 mL) and heptane (200 mL) and the layers separated. The aqueous layer was extracted with EtOAc (300 mL) and this organic layer was washed with brine (200 mL), then dried over Na₂SO₄, decanted and concentrated. The second organic layer was used to extract the brine wash and rinse the Na₂SO₄, then decanted and combined with the crude product. The solution was concentrated to dryness to give a clear colorless oil, rac-tert-butyl (1R,5R,6S)-6-hydroxy-6-methyl-2-azabicyclo[3.2.0]heptane-2-carboxylate (33.65 g, 96% yield). ESI-MS (M-tBu+H)⁺: 172.0. ¹H NMR (500 MHz, DMSO-d₆) δ ppm 4.68-4.85 (m, 1H), 3.74-3.95 (m, 1H), 3.35-3.51 (m, 2H), 2.63-2.77 (m, 1H), 2.20-2.32 (m, 1H), 2.05-2.16 (m, 1H), 1.60-1.79 (m, 2H), 1.32-1.43 (m, 9H), 1.21-1.28 (m, 3H).

### 2. Synthesis of rac-tert-butyl (1R,5R,6S)-6-((6-chloropyrazolo[1,5-a]pyrazin-4-yl)oxy)-6-methyl-2-azabicyclo[3.2.0]heptane-2-carboxylate

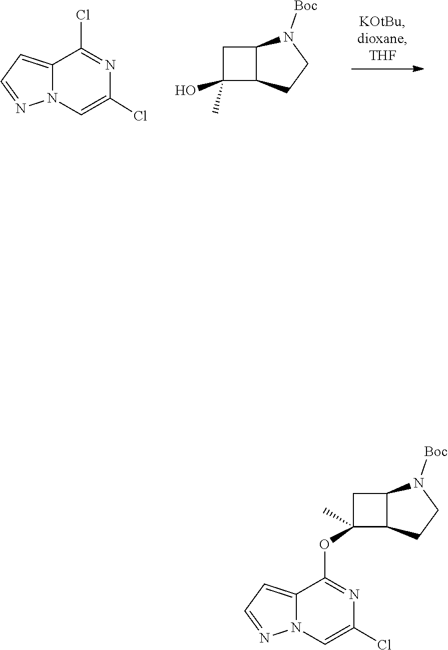 [SMILES: Clc1cn2nccc2c(Cl)n1 ; CC(C)(C)OC(=O)N1CC[C@@H]2[C@H]1C[C@]2(C)O ; CC(C)(C)OC(=O)N1CC[C@@H]2[C@H]1C[C@]2(C)Oc1nc(Cl)cn2nccc12]

To a round bottom flask containing rac-tert-butyl (1R,5R,6S)-6-hydroxy-6-methyl-2-azabicyclo[3.2.0]heptane-2-carboxylate (33.65 g, 148 mmol) was added dioxane (200 mL) followed by KOtBu (1 M in THF, 225 mL) while stirring at RT. The resulting dark orange solution was concentrated to dryness to remove solvent and tBuOH, leaving a tan solid mixture. This material was then dissolved in THF (150 mL) and placed in an ice bath, stirring for 20 mins. A solution of 4,6-dichloropyrazolo[1,5-a]pyrazine (35 g, 186 mmol) in THF (150 mL) was added dropwise over 20-30 mins and the reaction was stirred for 15 mins. 4,6-Dichloropyrazolo[1,5-a]pyrazine (5 g, 26.6 mmol) was added and the reaction stirred for 15 mins. Additional 4,6-dichloropyrazolo[1,5-a]pyrazine (3 g, 16.0 mmol) was added and the mixture stirred for 15 mins at RT, then concentrated to a low volume, diluted with water (400 mL) and extracted with EtOAc (2×300 mL). The aqueous layer was discarded and the combined organic layers were concentrated to dryness to give a thick dark brown oil. The material was purified by column chromatography (heptane to 50% EtOAc in heptane) to give a light orange solid, rac-tert-butyl (1R,5R,6S)-6-(6-chloropyrazolo[1,5-a]pyrazin-4-yl)oxy-6-methyl-2-azabicyclo[3.2.0]heptane-2-carboxylate (57.7 g, 95% yield). ESI-MS (M+H)⁺: 379.1. ¹H NMR (500 MHz, DMSO-d₆) δ ppm 8.68-8.71 (m, 1H), 8.07-8.13 (m, 1H), 6.92-6.98 (m, 1H), 3.96-4.12 (m, 1H), 3.36-3.49 (m, 2H), 3.13-3.27 (m, 1H), 2.64-2.72 (m, 1H), 2.19-2.29 (m, 1H), 2.07-2.18 (m, 1H), 1.84-1.96 (m, 1H), 1.71-1.77 (m, 3H), 1.33-1.39 (m, 9H).

### 3. Synthesis of rac-tert-butyl (1R,5R,6S)-6-methyl-6-((6-(1-methyl-1H-pyrazol-4-yl)pyrazolo[1,5-a]pyrazin-4-yl)oxy)-2-azabicyclo[3.2.0]heptane-2-carboxylate

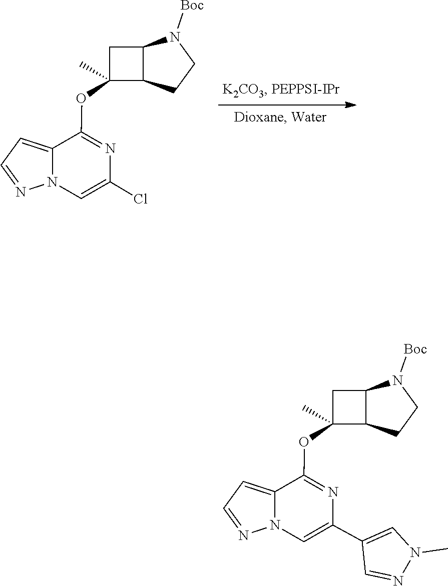 [SMILES: CC(C)(C)OC(=O)N1CC[C@@H]2[C@H]1C[C@]2(C)Oc1nc(Cl)cn2nccc12 ; Cn1cc(-c2cn3nccc3c(O[C@@]3(C)C[C@@H]4[C@H]3CCN4C(=O)OC(C)(C)C)n2)cn1]

To a round bottom flask containing rac-tert-butyl (1R,5R,6S)-6-(6-chloropyrazolo[1,5-a]pyrazin-4-yl)oxy-6-methyl-2-azabicyclo[3.2.0]heptane-2-carboxylate (55 g, 133.6 mmol) was added 1-methyl-4-(4,4,5,5-tetramethyl-1,3,2-dioxaborolan-2-yl)pyrazole (42 g, 201.9 mmol), K₂CO₃ (35 g, 253.25 mmol), dioxane (300 mL), and water (100 mL). This mixture was stirred for 10 mins at RT, then degassed with N₂ (3×). PEPPSI™-IPr catalyst (500 mg, 0.734 mmol) was added and the reaction was heated to reflux (95° C.) under N₂ for 45 mins. The reaction was then cooled to RT, water (300 mL) and EtOAc (200 mL) was added and the mixture stirred vigorously for 30 mins. The layers were separated, the aqueous layer was extracted with EtOAc (300 mL), then discarded and the combined organic layers concentrated in vacuo. The residual material was diluted with DCM (200 mL), filtered through Celite®, and concentrated to a low volume. The red solution was purified by column chromatography (25% EtOAc in heptane to EtOAc) to give rac-tert-butyl (1R,5R,6S)-6-methyl-6-((6-(1-methyl-1H-pyrazol-4-yl)pyrazolo[1,5-a]pyrazin-4-yl)oxy)-2-azabicyclo[3.2.0]heptane-2-carboxylate (55.6 g, 98% yield). ESI-MS (M+H)⁺: 425.1. ¹H NMR (500 MHz, DMSO-d₆) δ ppm 8.72-8.77 (m, 1H), 8.13-8.17 (m, 1H), 8.00-8.03 (m, 1H), 7.96-8.00 (m, 1H), 6.78-6.89 (m, 1H), 4.00-4.16 (m, 1H), 3.86-3.92 (m, 3H), 3.27-3.48 (m, 3H), 2.72-2.83 (m, 1H), 2.19-2.27 (m, 1H), 2.05-2.19 (m, 1H), 1.85-1.95 (m, 1H), 1.79 (s, 3H), 1.33-1.40 (m, 9H).

### 4. Synthesis of rac-1-((1R,5R,6S)-6-methyl-6-((6-(1-methyl-1H-pyrazol-4-yl)pyrazolo[1,5-a]pyrazin-4-yl)oxy)-2-azabicyclo[3.2.0]heptan-2-yl)prop-2-en-1-one

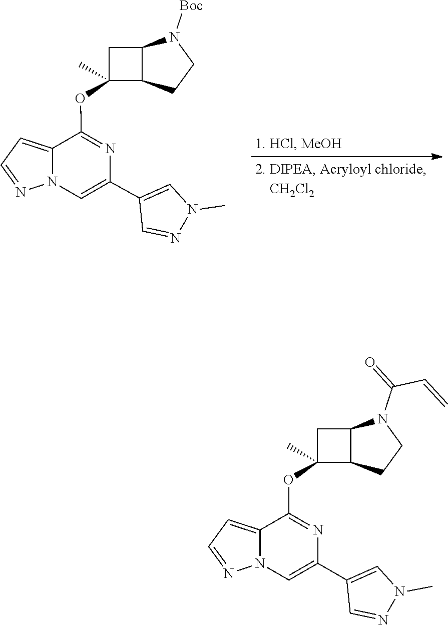 [SMILES: Cn1cc(-c2cn3nccc3c(O[C@@]3(C)C[C@@H]4[C@H]3CCN4C(=O)OC(C)(C)C)n2)cn1 ; C=CC(=O)N1CC[C@@H]2[C@H]1C[C@]2(C)Oc1nc(-c2cnn(C)c2)cn2nccc12]

To a round bottom flask containing rac-tert-butyl (1R,5R,6S)-6-methyl-6-((6-(1-methyl-1H-pyrazol-4-yl)pyrazolo[1,5-a]pyrazin-4-yl)oxy)-2-azabicyclo[3.2.0]heptane-2-carboxylate (55 g, 129.6 mmol) was added MeOH (300 mL), the solution cooled in an ice bath, then HCl (4 M in dioxane, 100 mL) added slowly over 15 mins. The reaction mixture was allowed to warm to RT and stirred for 90 mins. The reaction mixture was concentrated to a low volume to give a dark brown thick oil which was diluted with 3:1 EtOAc:EtOH (300 mL). This mixture was then heated to completely dissolve all solids while stirring vigorously, then allowed to cool to RT and concentrated to remove −100 mL solvent. The resulting slurry was filtered to collect the solids, rinsing with 100-150 mL 3:1 EtOAc:EtOH. The white solids were collected, dried via suction to give rac-4-(((1R,5R,6S)-6-methyl-2-azabicyclo[3.2.0]heptan-6-yl)oxy)-6-(1-methylpyrazol-4-yl)pyrazolo[1,5-a]pyrazine hydrochloride (42.5 g, 86% yield). ESI-MS (M+H)⁺: 325.1.

A solution of this solid (42 g, 116 mmol) in DCM (400 mL) and DIPEA (65 mL, 373.2 mmol) was cooled on a dry ice/acetonitrile bath for 20 mins, then acryloyl chloride (11.14 g, 123.1 mmol) was added dropwise over 15 mins. The reaction was allowed to stir for 10 mins, then concentrated to remove half the solvent and purified directly via chromatography (heptane to EtOAc) to give a light tan foam solid, rac-1-((1R,5R,6S)-6-methyl-6-(6-(1-methylpyrazol-4-yl)pyrazolo[1,5-a]pyrazin-4-yl)oxy-2-azabicyclo[3.2.0]heptan-2-yl)prop-2-en-1-one (34.2 g, 72% yield). ESI-MS (M+H)⁺: 379.2. ¹H NMR (500 MHz, DMSO-d₆) δ ppm 8.72-8.75 (m, 1H), 8.11-8.18 (m, 1H), 7.99-8.02 (m, 1H), 7.95-7.99 (m, 1H), 6.79-6.85 (m, 1H), 6.44-6.59 (m, 1H), 6.05-6.16 (m, 1H), 5.59-5.70 (m, 1H), 4.31-4.43 (m, 1H), 3.89 (s, 3H), 3.61-3.78 (m, 1H), 3.43-3.56 (m, 1H), 3.28-3.37 (m, 1H), 2.82-2.97 (m, 1H), 2.08-2.35 (m, 2H), 1.89-2.06 (m, 1H), 1.79-1.84 (m, 3H).

### 5. Isolation of 1-((1R,5R,6S)-6-methyl-6-((6-(1-methyl-1H-pyrazol-4-yl)pyrazolo[1,5-a]pyrazin-4-yl)oxy)-2-azabicyclo[3.2.0]heptan-2-yl)prop-2-en-1-one and 1-((1S,5S,6R)-6-methyl-6-((6-(1-methyl-1H-pyrazol-4-yl)pyrazolo[1,5-a]pyrazin-4-yl)oxy)-2-azabicyclo[3.2.0]heptan-2-yl)prop-2-en-1-one

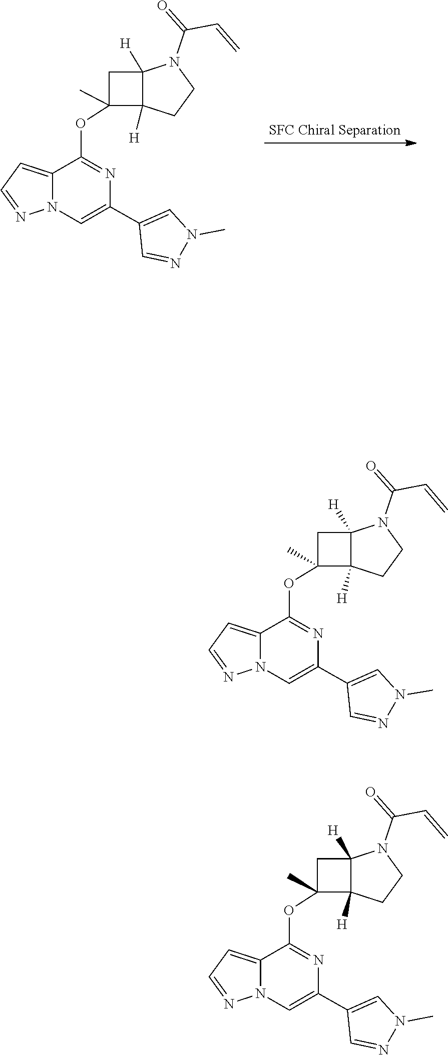 [SMILES: C=CC(=O)N1CCC2C1CC2(C)Oc1nc(-c2cnn(C)c2)cn2nccc12 ; C=CC(=O)N1CC[C@@H]2[C@H]1C[C@]2(C)Oc1nc(-c2cnn(C)c2)cn2nccc12 ; C=CC(=O)N1CC[C@H]2[C@@H]1C[C@@]2(C)Oc1nc(-c2cnn(C)c2)cn2nccc12]

34.2 g of the racemic mixture of isomers was resolved by chiral SFC purification ((S,S) Whelk 0-1 [Regis Technologies] 2.1×25 cm, 45% EtOH with 0.25% Et₃N in CO₂, flow rate: 80 g/min, System pressure 100 bar, column temp 25° C.) to afford the two products as a first eluting peak (E1) and second eluting peak (E2) as colorless oils. The peaks were analyzed by an analytic SFC method ((S,S) Whelk 0-1 [Regis Technologies] 4.6×100 mm, 5-65% EtOH with 0.1% isopropylamine in CO₂, flow rate: 4 mL/min, System pressure 125 bar, column temp 40° C.) to identify Peak 1 (Example 190) (2.6 min, 14.27 g, 98.3% ee, 99.7% purity), and a second eluting peak (Example 191) (E2) Peak 2 (2.9 min, 10.73 g, 98.8% ee, 98.0% purity).

### Examples 192, 193, 194 & 195: ((1R,5R,6R)-6-methyl-6-((6-(1-methyl-1H-pyrazol-4-yl)pyrazolo[1,5-a]pyrazin-4-yl)oxy)-2-azabicyclo[3.2.0]heptan-2-yl)((2R,3S)-3-methyloxiran-2-yl)methanone and ((1R,5R,6S)-6-methyl-6-((6-(1-methyl-1H-pyrazol-4-yl)pyrazolo[1,5-a]pyrazin-4-yl)oxy)-2-azabicyclo[3.2.0]heptan-2-yl)((2S,3R)-3-methyloxiran-2-yl)methanone and ((1S,5S,6R)-6-methyl-6-((6-(1-methyl-1H-pyrazol-4-yl)pyrazolo[1,5-a]pyrazin-4-yl)oxy)-2-azabicyclo[3.2.0]heptan-2-yl)((2R,3S)-3-methyloxiran-2-yl)methanone and ((1R,5R,6S)-6-methyl-6-((6-(1-methyl-1H-pyrazol-4-yl)pyrazolo[1,5-a]pyrazin-4-yl)oxy)-2-azabicyclo[3.2.0]heptan-2-yl)((2S,3R)-3-methyloxiran-2-yl)methanone

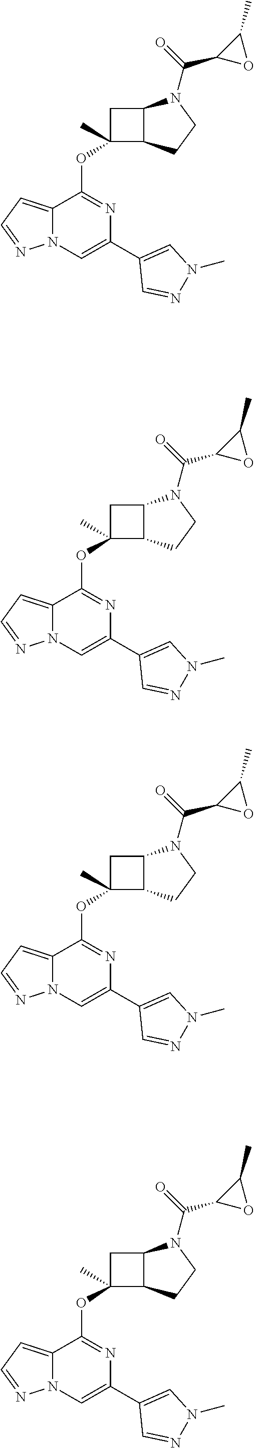 [SMILES: C[C@@H]1O[C@H]1C(=O)N1CC[C@@H]2[C@H]1C[C@@]2(C)Oc1nc(-c2cnn(C)c2)cn2nccc12 ; C[C@H]1O[C@@H]1C(=O)N1CC[C@H]2[C@@H]1C[C@]2(C)Oc1nc(-c2cnn(C)c2)cn2nccc12 ; C[C@@H]1O[C@H]1C(=O)N1CC[C@H]2[C@@H]1C[C@@]2(C)Oc1nc(-c2cnn(C)c2)cn2nccc12 ; C[C@H]1O[C@@H]1C(=O)N1CC[C@@H]2[C@H]1C[C@]2(C)Oc1nc(-c2cnn(C)c2)cn2nccc12]

### 1. Synthesis of rac-tert-butyl (1R,5R,6S)-6-((6-chloropyrazolo[1,5-a]pyrazin-4-yl)oxy)-6-methyl-2-azabicyclo[3.2.0]heptane-2-carboxylate

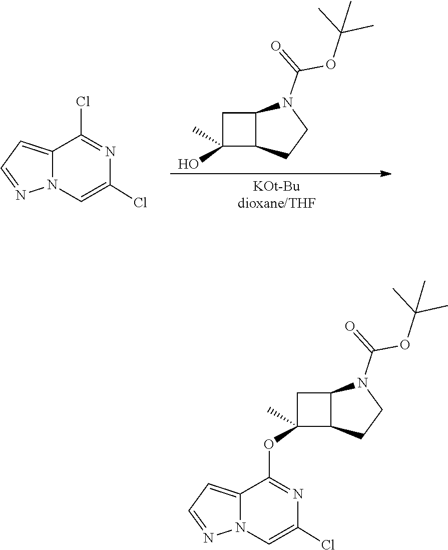 [SMILES: Clc1cn2nccc2c(Cl)n1 ; CC(C)(C)OC(=O)N1CC[C@@H]2[C@H]1C[C@]2(C)O ; CC(C)(C)OC(=O)N1CC[C@@H]2[C@H]1C[C@]2(C)Oc1nc(Cl)cn2nccc12]

To a round-bottom flask containing tert-butyl 6-hydroxy-6-methyl-2-azabicyclo[3.2.0]heptane-2-carboxylate (33.7 g, 148 mmol) was added dioxane (200 mL) followed by KOtBu (1.0 M in THF, 225 mL) while stirring at RT. The resulting solution was concentrated to dryness and the residue dissolved in THF (150 mL) and placed in an ice bath, stirring for 20 mins. A solution of 4,6-dichloropyrazolo[1,5-a]pyrazine (35.0 g, 186 mmol) in THF (150 mL) was added dropwise over 30 mins and the mixture stirred for 15 mins. Additional 4,6-dichloropyrazolo[1,5-a]pyrazine (5.0 g, 27 mmol) was added and after another 15 mins, further 4,6-dichloropyrazolo[1,5-a]pyrazine (3.0 g, 16 mmol) was added and the reaction stirred for 15 mins. The reaction mixture was concentrated to a low volume, diluted with water (400 mL) and extracted with EtOAc (2×300 mL). The combined organic layers were concentrated to dryness to give a thick, dark brown oil. This material was purified via silica gel chromatography (heptane to 50% EtOAc in heptane) to give rac-tert-butyl (1R,5R,6S)-6-((6-chloropyrazolo[1,5-a]pyrazin-4-yl)oxy)-6-methyl-2-azabicyclo[3.2.0]heptane-2-carboxylate as a light orange solid, (57.7 g, 95% yield). LC-MS: m/z=278.1 (M-CO₂t-Bu+H)⁺.

### 2. Synthesis of rac-tert-butyl (1R,5R,6S)-6-methyl-6-((6-(1-methyl-1H-pyrazol-4-yl)pyrazolo[1,5-a]pyrazin-4-yl)oxy)-2-azabicyclo[3.2.0]heptane-2-carboxylate

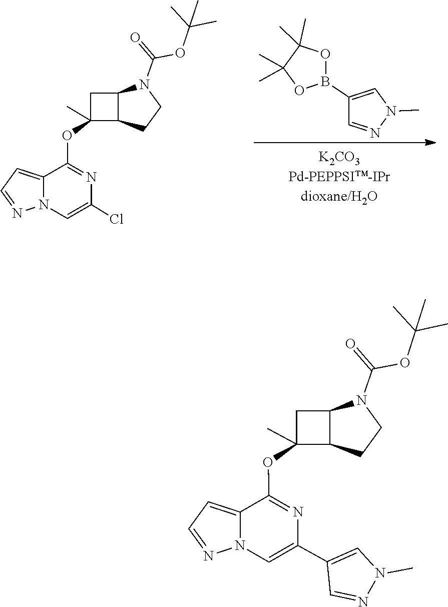

To a round-bottom flask containing rac-tert-butyl (1R,5R,6S)-6-((6-chloropyrazolo[1,5-a]pyrazin-4-yl)oxy)-6-methyl-2-azabicyclo[3.2.0]heptane-2-carboxylate (55.0 g, 134 mmol) was added 1-methyl-4-(4,4,5,5-tetramethyl-1,3,2-dioxaborolan-2-yl)pyrazole (42.0 g, 202 mmol), K₂CO₃ (35.0 g, 253 mmol), dioxane (300 mL) and water (100 mL) and the mixture was stirred for 10 mins at RT, then degassed with N₂. Pd-PEPPSI™-IPr (500 mg, 0.734 mmol) was added and the reaction was heated to 95° C. under N₂ for 45 mins. The reaction was cooled to RT, water (300 mL) and EtOAc (200 mL) were added and the mixture stirred vigorously for 30 mins. The layers were separated, the aqueous layer was extracted with EtOAc (300 mL), the combined organic layers were concentrated to give a thick dark red/brown oil. This material was diluted with DCM (200 mL), filtered through a Celite® pad and concentrated to a low volume. The red solution was purified by silica gel chromatography (25% EtOAc in heptane to EtOAc) to give rac-tert-butyl (1R,5R,6S)-6-methyl-6-((6-(1-methyl-1H-pyrazol-4-yl)pyrazolo[1,5-a]pyrazin-4-yl)oxy)-2-azabicyclo[3.2.0]heptane-2-carboxylate (55.6 g, 98% yield). LC-MS: m/z=447.1 (M+Na)+

### 3. Synthesis of rac-6-(1-methyl-1H-pyrazol-4-yl)-4-(((1R,5R,6S)-6-methyl-2-azabicyclo[3.2.0]heptan-6-yl)oxy)pyrazolo[1,5-a]pyrazine

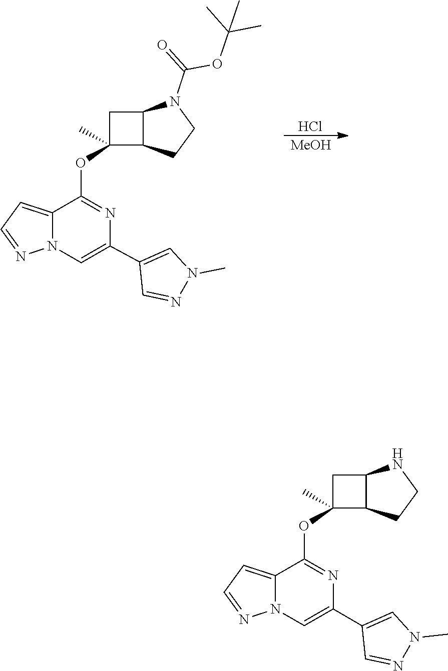 [SMILES: "MeOH" ; Cn1cc(-c2cn3nccc3c(O[C@@]3(C)C[C@@H]4[C@H]3CCN4C(=O)OC(C)(C)C)n2)cn1 ; Cl ; Cn1cc(-c2cn3nccc3c(O[C@@]3(C)C[C@H]4NCC[C@H]43)n2)cn1]

To a round-bottom flask containing rac-tert-butyl (1R,5R,6S)-6-methyl-6-((6-(1-methyl-1H-pyrazol-4-yl)pyrazolo[1,5-a]pyrazin-4-yl)oxy)-2-azabicyclo[3.2.0]heptane-2-carboxylate (55.0 g, 130 mmol) was added MeOH (300 mL), the solution cooled in an ice bath for 15 mins, and then HCl (4.0 M in dioxane, 100 mL) was added slowly over 15 mins. The reaction mixture was allowed to warm to RT and stirred for 90 mins. The reaction mixture was concentrated to a low volume to give a thick, dark brown oil. The mixture was diluted with 3:1 EtOAc:EtOH (300 mL), this mixture was then heated to completely dissolve all solids while stirring vigorously. The brown solution was allowed to cool to RT and concentrated to remove −100 mL solvent. The resulting slurry was filtered to collect the solids, rinsing with 3:1 EtOAc:EtOH (100-150 mL) to give rac-6-(1-methyl-1H-pyrazol-4-yl)-4-(((1R,5R,6S)-6-methyl-2-azabicyclo[3.2.0]heptan-6-yl)oxy)pyrazolo[1,5-a]pyrazine (42.5 g, 86% yield), which was used without further purification.

### 4. Synthesis of ((1R,5R,6S)-6-methyl-6-((6-(1-methyl-1H-pyrazol-4-yl)pyrazolo[1,5-a]pyrazin-4-yl)oxy)-2-azabicyclo[3.2.0]heptan-2-yl)((2R,3S)-3-methyloxiran-2-yl)methanone, ((1R,5R,6S)-6-methyl-6-((6-(1-methyl-1H-pyrazol-4-yl)pyrazolo[1,5-a]pyrazin-4-yl)oxy)-2-azabicyclo[3.2.0]heptan-2-yl)((2S,3R)-3-methyloxiran-2-yl)methanone, ((1S,5S,6R)-6-methyl-6-((6-(1-methyl-1H-pyrazol-4-yl)pyrazolo[1,5-a]pyrazin-4-yl)oxy)-2-azabicyclo[3.2.0]heptan-2-yl)((2R,3S)-3-methyloxiran-2-yl)methanone and ((1S,5S,6R)-6-methyl-6-((6-(1-methyl-1H-pyrazol-4-yl)pyrazolo[1,5-a]pyrazin-4-yl)oxy)-2-azabicyclo[3.2.0]heptan-2-yl)((2R,3R)-3-methyloxiran-2-yl)methanone

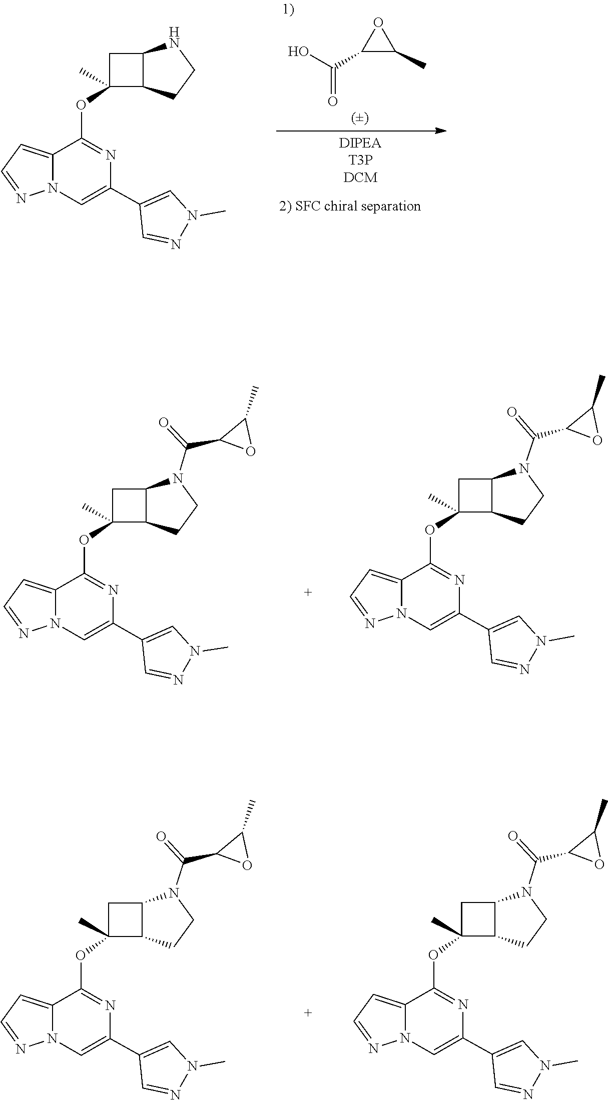

To a vial was added rac-6-(1-methyl-1H-pyrazol-4-yl)-4-(((1R,5R,6S)-6-methyl-2-azabicyclo[3.2.0]heptan-6-yl)oxy)pyrazolo[1,5-a]pyrazine (150 mg, 0.46 mmol), DCM (4.0 mL), DIPEA (200 μL, 1.15 mmol) and rac-(2R,3S)-3-methyloxirane-2-carboxylic acid (90.0 mg, 0.882 mmol) followed by T3P (600 mg, 0.943 mmol, 50% solution in DMF), the reaction was sealed and stirred at 30° C. for 1 h. The mixture was purified directly by silica gel chromatography (heptane to EtOAc to 3:1 EtOAc:EtOH) to give a clear colorless film, rac-((1R,5R,6R)-6-methyl-6-((6-(1-methyl-1H-pyrazol-4-yl)pyrazolo[1,5-a]pyrazin-4-yl)oxy)-2-azabicyclo[3.2.0]heptan-2-yl)((2S,3R)-3-methyloxiran-2-yl)methanone (180 mg, 95% yield).

This material (180 mg) was dissolved in MeOH (18 mL) and separated by chiral SFC (CHIRALPAK IA 30×250 mm, 5 μm column; 20% (1:1) MeOH:DCM w/0.1% DMEA in CO₂); flow rate=100 mL/min, ABPR 120 bar, MBPR 40 psi, column temp. 40° C.) to provide:

### Example 194, Peak 1, the first eluting isomer (E1); (32.0 mg, 92.7% ee, Rf=2.98 min). LC/MS: m/z=431.3 (M+Na)⁺

### Example 195, Peak 2, the second eluting isomer (E2); (27.0 mg, 98.9% ee, Rf=4.11 min). LC/MS: m/z=431.3 (M+Na)⁺

Together with a mixture of two compounds (Rf=2.61 min) which was resubjected to chiral SFC (LUX Cellulose-4 LC 30×250 mm, 3 μm column; 30% (1:1) MeOH:DCM w/0.1% DMEA in CO₂); flow rate=100 mL/min, ABPR 120 bar, MBPR 40 psi, column temp. 40° C.) to provide:

### Example 192, Peak 3, the third eluting isomer (E3); (42.3 mg, 98.7% ee, Rf=2.54 min). LC/MS: m/z=431.3 (M+Na)+

### Example 193, Peak 4, the fourth eluting isomer (E4); (39.7 mg, 100% ee, Rf=2.62 min). LC/MS: m/z=431.3 (M+Na)⁺. ¹H NMR (600 MHz, DMSO-d₆) δ ppm=1.23-1.33 (m, 3H) 1.82 (d, J=14.53 Hz, 3H) 1.88-2.08 (m, 1H) 1.88-2.09 (m, 1H) 2.10-2.44 (m, 3H) 2.80-3.06 (m, 1H) 3.45-3.53 (m, 1H) 3.55-3.85 (m, 2H) 3.89 (s, 3H) 4.27-4.45 (m, 1H) 6.82 (br s, 1H) 7.97 (s, 1H) 8.00 (d, J=1.45 Hz, 1H) 8.14 (s, 1H) 8.72 (s, 1H)

The absolute and relative stereochemistry of the separated products was assigned arbitrarily.

### Example 196: Cyclobut-1-en-1-yl((1R,5R,6S)-6-methyl-6-((6-(2-methylthiazol-5-yl)pyrazolo[1,5-a]pyrazin-4-yl)oxy)-2-azabicyclo[3.2.0]heptan-2-yl)methanone and cyclobut-1-en-1-yl((1S,5S,6R)-6-methyl-6-((6-(2-methylthiazol-5-yl)pyrazolo[1,5-a]pyrazin-4-yl)oxy)-2-azabicyclo [3.2.0]heptan-2-yl)methanone

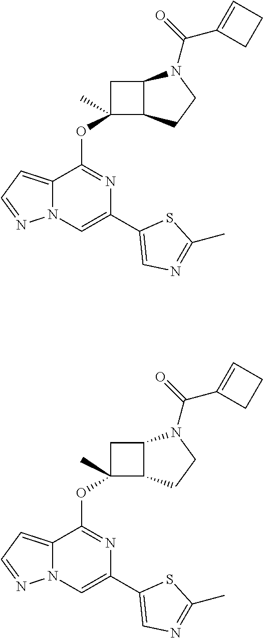 [SMILES: Cc1ncc(-c2cn3nccc3c(O[C@@]3(C)C[C@@H]4[C@H]3CCN4C(=O)C3=CCC3)n2)s1 ; Cc1ncc(-c2cn3nccc3c(O[C@]3(C)C[C@H]4[C@@H]3CCN4C(=O)C3=CCC3)n2)s1]

### 1. Synthesis of racemic tert-butyl (1R,5R,6S)-6-methyl-6-((6-(2-methylthiazol-5-yl)pyrazolo[1,5-a]pyrazin-4-yl)oxy)-2-azabicyclo[3.2.0]heptane-2-carboxylate

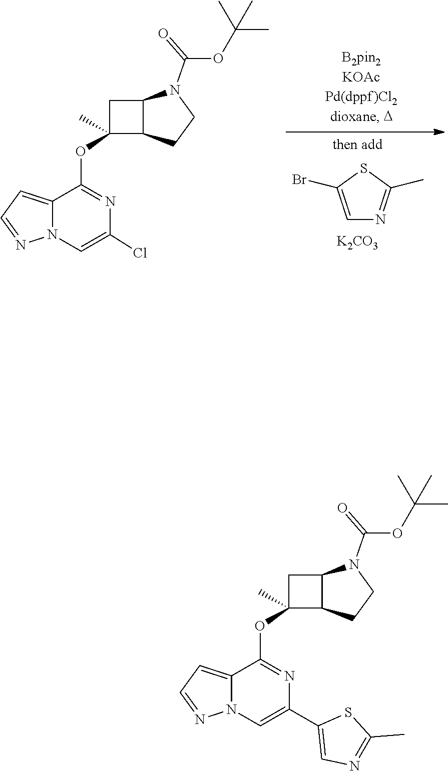

To a vial containing racemic tert-butyl (1R,5R,6S)-6-((6-chloropyrazolo[1,5-a]pyrazin-4-yl)oxy)-6-methyl-2-azabicyclo[3.2.0]heptane-2-carboxylate (Step 1, Example 192, 2.7 g, 7.1 mmol) was added dioxane (15 mL) and the solution was purged with N₂ for 10 mins. Pd(dppf)Cl₂:CH₂Cl₂ (500 mg, 0.612 mmol), KOAc (2.0 g, 20.4 mmol), and bis(pinacolato)diboron (3.5 g, 13.8 mmol) were added, the mixture purged with N₂, the vial then sealed and heated at 90° C. for 7 h. The crude reaction mixture was transferred to a vial containing K₂CO₃ (2.0 g, 14.5 mmol) and 5-bromo-2-methyl-thiazole (1.5 g, 8.4 mmol), water (5.0 mL) was added, and the vial was sealed and placed on a 90° C. stir plate. The reaction was stirred for 45 mins, then allowed to cool to RT overnight. The reaction mixture was diluted with water (20 mL) and extracted with EtOAc (3×25 mL). The combined organic layers were concentrated to dryness and purified by silica gel chromatography (heptane to EtOAc) to give a clear yellow oil, racemic tert-butyl (1R,5R,6S)-6-methyl-6-((6-(2-methylthiazol-5-yl)pyrazolo[1,5-a]pyrazin-4-yl)oxy)-2-azabicyclo[3.2.0]heptane-2-carboxylate (2.5 g, 56% yield) LCMS: m/z=442.3 (M+H)⁺.

### 2. Synthesis of racemic 2-methyl-5-(4-(((1R,5R,6S)-6-methyl-2-azabicyclo[3.2.0]heptan-6-yl)oxy)pyrazolo[1,5-a]pyrazin-6-yl)thiazole hydrochloride

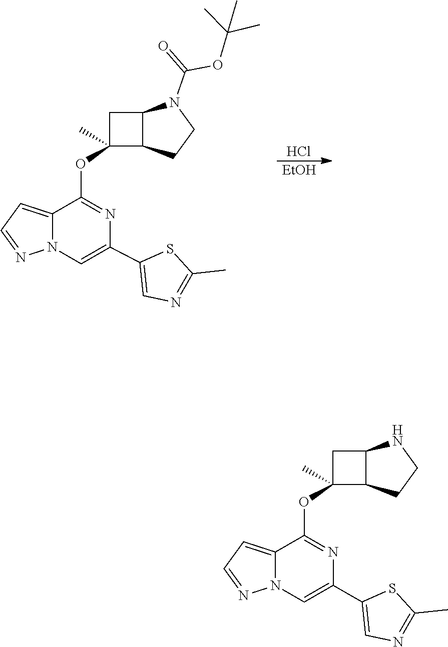 [SMILES: "EtOH" ; Cc1ncc(-c2cn3nccc3c(O[C@@]3(C)C[C@@H]4[C@H]3CCN4C(=O)OC(C)(C)C)n2)s1 ; Cl ; Cc1ncc(-c2cn3nccc3c(O[C@@]3(C)C[C@H]4NCC[C@H]43)n2)s1]

To a vial containing racemic tert-butyl (1R,5R,6S)-6-methyl-6-((6-(2-methylthiazol-5-yl)pyrazolo[1,5-a]pyrazin-4-yl)oxy)-2-azabicyclo[3.2.0]heptane-2-carboxylate (2.5 g, 4.0 mmol) was added HCl (1.25 M in EtOH, 15 mL) and the reaction stirred at 30° C., overnight. The reaction mixture was warmed to 35° C. and allowed to stir for an additional 5 h. The reaction was diluted with EtOAc (15 mL), the solids were collected by filtration through filter paper, rinsing with EtOAc (10 mL) and then dried to afford racemic 2-methyl-5-(4-(((1R,5R,6S)-6-methyl-2-azabicyclo[3.2.0]heptan-6-yl)oxy)pyrazolo[1,5-a]pyrazin-6-yl)thiazole hydrochloride (1.33 g, 89% yield). LC-MS: m/z=342.3 (M+H)⁺.

### 3. Synthesis of cyclobut-1-en-1-yl((R,5R,6S)-6-methyl-6-((6-(2-methylthiazol-5-yl)pyrazolo[1,5-a]pyrazin-4-yl)oxy)-2-azabicyclo[3.2.0]heptan-2-yl)methanone

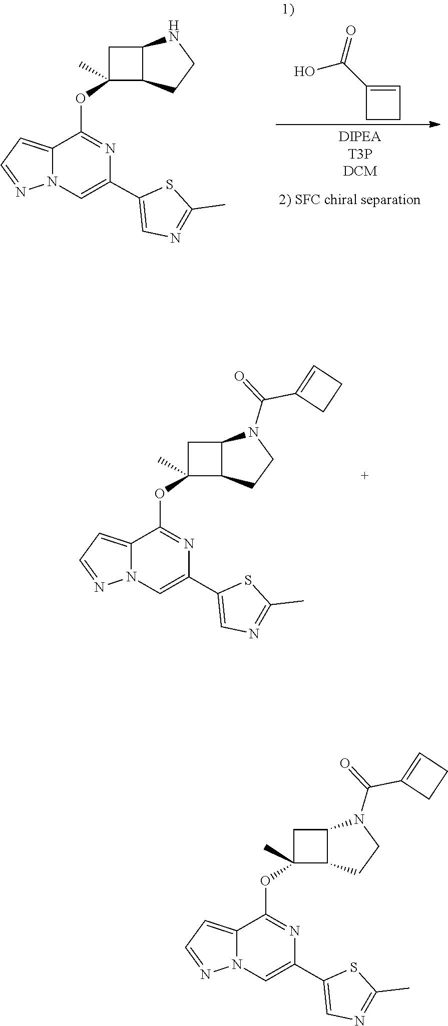 [SMILES: Cc1ncc(-c2cn3nccc3c(O[C@@]3(C)C[C@H]4NCC[C@H]43)n2)s1 ; O=C(O)C1=CCC1 ; Cc1ncc(-c2cn3nccc3c(O[C@@]3(C)C[C@@H]4[C@H]3CCN4C(=O)C3=CCC3)n2)s1 ; Cc1ncc(-c2cn3nccc3c(O[C@]3(C)C[C@H]4[C@@H]3CCN4C(=O)C3=CCC3)n2)s1]

To a vial was added cyclobutene-1-carboxylic acid (51.9 mg, 0.53 mmol) followed by a stock solution containing racemic 2-methyl-5-(4-(((1R,5R,6S)-6-methyl-2-azabicyclo[3.2.0]heptan-6-yl)oxy)pyrazolo[1,5-a]pyrazin-6-yl)thiazole hydrochloride (100 mg, 0.26 mmol), DCM (3.0 mL) and DIPEA (150 μL 0.861 mmol). To this mixture was added T3P (168 mg, 0.264 mmol, 50% solution in DMF) while stirring at RT. The vial was sealed, then stirred at 35° C. overnight. The reaction was concentrated to dryness and purified by silica gel chromatography (heptane to 3:1 EtOAc:EtOH) to give racemic cyclobut-1-en-1-yl((1R,5R,6S)-6-methyl-6-((6-(2-methylthiazol-5-yl)pyrazolo[1,5-a]pyrazin-4-yl)oxy)-2-azabicyclo[3.2.0]heptan-2-yl)methanone a clear tan oil, (70.0 mg, 63% yield). LCMS: m/z=422.2 (M+H)⁺. ¹H NMR (500 MHz, DMSO-d₆) δ ppm=1.81 (d, J=6.71 Hz, 3H) 1.85-1.94 (m, 1H) 1.98-2.18 (m, 1H) 2.23-2.31 (m, 1H) 2.32-2.42 (m, 3H) 2.58-2.64 (m, 1H) 2.67 (s, 3H) 2.78-2.91 (m, 1H) 3.22-3.46 (m, 1H) 3.52-3.66 (m, 1H) 3.69-3.82 (m, 1H) 4.31-4.45 (m, 1H) 6.49 (d, J=19.53 Hz, 1H) 6.89-6.94 (m, 1H) 7.95 (s, 1H) 8.10 (d, J=2.44 Hz, 1H) 8.25 (s, 1H) 9.08 (d, J=4.88 Hz, 1H).

This racemic material was further purified by chiral SFC (CHIRALPAK IB 30×250 mm, 5 μm column; 20% MeOH in CO₂; flow rate=100 mL/min, ABPR 120 bar, MBPR 40 psi, column temp. 40° C.), to provide the first eluting peak (Example 196), E1, (1.0 mg, 100% ee, Rf=2.81 min) and the second eluting peak, E2 (1.0 mg, 93.1% ee, Rf=2.96 min).

### Examples 197 & 198: 1-((1S,5R,6R)-6-((3-methyl-6-(1-methyl-1H-pyrazol-4-yl)pyrazolo[1,5-a]pyrazin-4-yl)oxy)-3-azabicyclo[3.2.0]heptan-3-yl)prop-2-en-1-one and 1-((1R,5S,6S)-6-((3-methyl-6-(1-methyl-1H-pyrazol-4-yl)pyrazolo[1,5-a]pyrazin-4-yl)oxy)-3-azabicyclo[3.2.0]heptan-3-yl)prop-2-en-1-one

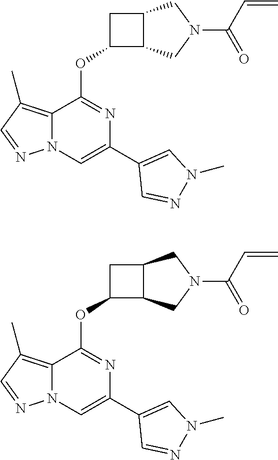 [SMILES: C=CC(=O)N1C[C@H]2C[C@@H](Oc3nc(-c4cnn(C)c4)cn4ncc(C)c34)[C@H]2C1 ; C=CC(=O)N1C[C@@H]2C[C@H](Oc3nc(-c4cnn(C)c4)cn4ncc(C)c34)[C@@H]2C1]

### 1. Synthesis of rac-tert-butyl (1R,5S,6S)-6-((3-iodo-6-(1-methyl-1H-pyrazol-4-yl)pyrazolo[1,5-a]pyrazin-4-yl)oxy)-3-azabicyclo[3.2.0]heptane-3-carboxylate

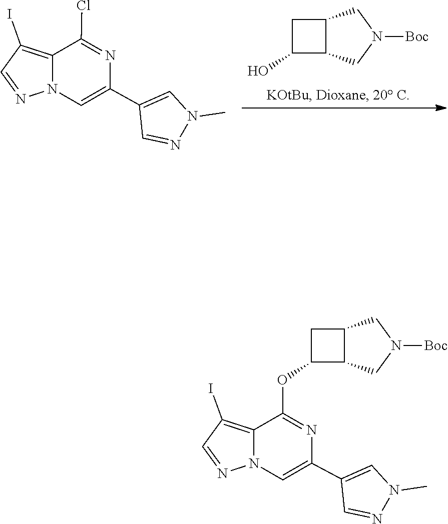 [SMILES: Cn1cc(-c2cn3ncc(I)c3c(Cl)n2)cn1 ; CC(C)(C)OC(=O)N1C[C@H]2C[C@@H](O)[C@H]2C1 ; Cn1cc(-c2cn3ncc(I)c3c(O[C@@H]3C[C@@H]4CN(C(=O)OC(C)(C)C)C[C@@H]43)n2)cn1]

To a vial was added 4-chloro-3-iodo-6-(1-methylpyrazol-4-yl)pyrazolo[1,5-a]pyrazine (350 mg, 0.973 mol), rac-tert-butyl (1S,5R,6R)-6-hydroxy-3-azabicyclo[3.2.0]heptane-3-carboxylate (250 mg, 1.17 mmol), and THF (2 mL). This mixture was stirred at RT for 5 mins, then KOtBu (1.0 M, 1 mL) was added. The reaction was allowed to stir for 10 mins at RT, then concentrated to dryness. The crude material was purified directly by column chromatography (heptane to EtOAc) to give an off-white solid, tert-butyl (1S,5R,6R)-6-(3-iodo-6-(1-methylpyrazol-4-yl)pyrazolo[1,5-a]pyrazin-4-yl)oxy-3-azabicyclo[3.2.0]heptane-3-carboxylate (490 mg, 94%). ESI-MS (M+H)⁺: 537.0. ¹H NMR (DMSO-d₆) δ: 8.81 (s, 1H), 8.19 (s, 1H), 8.12 (br s, 1H), 7.99 (s, 1H), 5.35 (q, J=7.9 Hz, 1H), 3.82-3.90 (m, 4H), 3.38-3.54 (m, 2H), 3.06-3.28 (m, 2H), 2.70-2.89 (m, 2H), 1.83-1.99 (m, 1H), 1.26-1.50 (m, 9H).

### 2. Synthesis of rac-tert-butyl (1S,5R,6R)-6-((3-methyl-6-(1-methyl-1H-pyrazol-4-yl)pyrazolo[1,5-a]pyrazin-4-yl)oxy)-3-azabicyclo[3.2.0]heptane-3-carboxylate

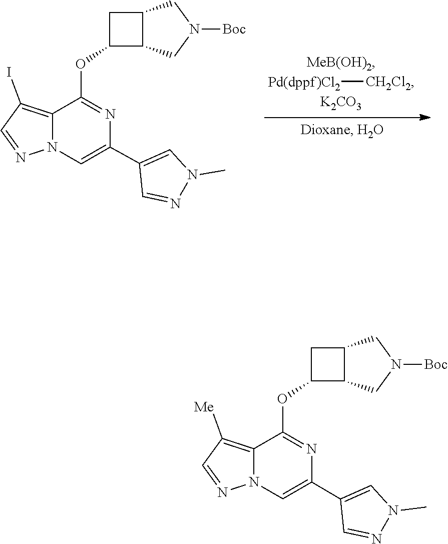

To a microwave vial was added methylboronic acid (273 mg, 4.57 mmol), potassium carbonate (250 mg, 1.81 mmol), and Pd(dppf)Cl₂:CH₂Cl₂ (25 mg, 0.031 mmol) followed by a solution of rac-tert-butyl (1S,5R,6R)-6-(3-iodo-6-(1-methylpyrazol-4-yl)pyrazolo[1,5-a]pyrazin-4-yl)oxy-3-azabicyclo[3.2.0]heptane-3-carboxylate (490 mg, 0.914 mmol) in dioxane (9 mL) and water (2 mL). The vial was sealed then heated to 120° C. for 1 h in a Biotage microwave reactor. The reaction was cooled to RT then diluted with water (10 mL) and extracted with EtOAc (4×10 mL). The combined organic layers were concentrated to dryness and purified via silica gel column chromatography (heptane to EtOAc) to give a white solid, tert-butyl (1S,5R,6R)-6-(3-methyl-6-(1-methylpyrazol-4-yl)pyrazolo[1,5-a]pyrazin-4-yl)oxy-3-azabicyclo[3.2.0]heptane-3-carboxylate (330 mg, 64%). ESI-MS (M+H)⁺: 425.1. ¹H NMR (DMSO-d₆) δ: 8.62 (s, 1H), 8.16 (s, 1H), 7.97 (s, 1H), 7.74-7.89 (m, 1H), 5.42 (br d, J=7.9 Hz, 1H), 3.84-3.91 (m, 3H), 3.74-3.83 (m, 1H), 3.36-3.52 (m, 2H), 3.06-3.23 (m, 2H), 2.67-2.90 (m, 2H), 2.35-2.48 (m, 2H), 1.73-1.86 (m, 1H), 1.27-1.49 (m, 9H).

### 3. Synthesis of rac-1-((1S,5R,6R)-6-((3-methyl-6-(1-methyl-1H-pyrazol-4-yl)pyrazolo[1,5-a]pyrazin-4-yl)oxy)-3-azabicyclo[3.2.0]heptan-3-yl)prop-2-en-1-one hydrochloride

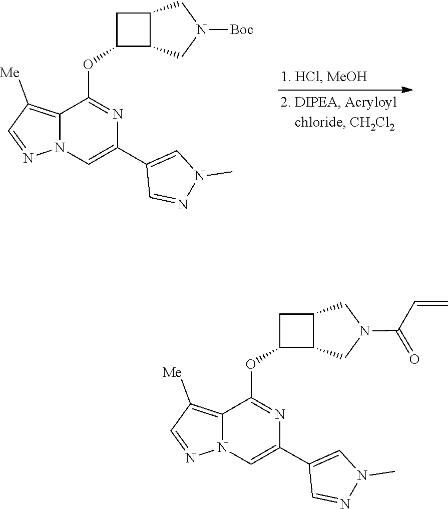 [SMILES: Cc1cnn2cc(-c3cnn(C)c3)nc(O[C@@H]3C[C@@H]4CN(C(=O)OC(C)(C)C)C[C@@H]43)c12 ; C=CC(=O)N1C[C@H]2C[C@@H](Oc3nc(-c4cnn(C)c4)cn4ncc(C)c34)[C@H]2C1]

To a vial containing rac-tert-butyl (1S,5R,6R)-6-(3-methyl-6-(1-methylpyrazol-4-yl)pyrazolo[1,5-a]pyrazin-4-yl)oxy-3-azabicyclo[3.2.0]heptane-3-carboxylate (330 mg, 0.583 mmol) was added HCl (1.25 M, 6 mL). The mixture was heated to 45° C. for 10 mins, MeOH (10 mL) was added and the reaction left to stand at 45° C. for 3 h. The reaction mixture was concentrated to dryness to give a white solid, rac-4-(((1S,5R,6R)-3-azabicyclo[3.2.0]heptan-6-yl)oxy)-3-methyl-6-(1-methyl-1H-pyrazol-4-yl)pyrazolo[1,5-a]pyrazine hydrochloride (300 mg, crude) that was used without further purification. ESI-MS (M+H)⁺: 325.1.

To a vial containing rac-4-(((1S,5R,6R)-3-azabicyclo[3.2.0]heptan-6-yl)oxy)-3-methyl-6-(1-methyl-1H-pyrazol-4-yl)pyrazolo[1,5-a]pyrazine hydrochloride (50 mg, 0.154 mmol) was added DCM (3 mL) and DIPEA (74.2 mg, 0.574 mmol) while stirring at RT. The reaction was cooled to −20° C., then acryloyl chloride (13.6 mg, 0.154 mmol) was added. After stirring for 10 mins, the reaction was purified by chromatography (heptane to 3:1 EtOAc:EtOH) to afford rac-1-((1S,5R,6R)-6-((3-methyl-6-(1-methyl-1H-pyrazol-4-yl)pyrazolo[1,5-a]pyrazin-4-yl)oxy)-3-azabicyclo[3.2.0]heptan-3-yl)prop-2-en-1-one as a tan oil (65 mg, crude), that was used without further purification. ESI-MS (M+H)⁺: 379.1.

### 4. Isolation of 1-((1S,5R,6R)-6-((3-methyl-6-(1-methyl-1H-pyrazol-4-yl)pyrazolo[1,5-a]pyrazin-4-yl)oxy)-3-azabicyclo[3.2.0]heptan-3-yl)prop-2-en-1-one and 1-((1R,5S,6S)-6-((3-methyl-6-(1-methyl-1H-pyrazol-4-yl)pyrazolo[1,5-a]pyrazin-4-yl)oxy)-3-azabicyclo[3.2.0]heptan-3-yl)prop-2-en-1-one

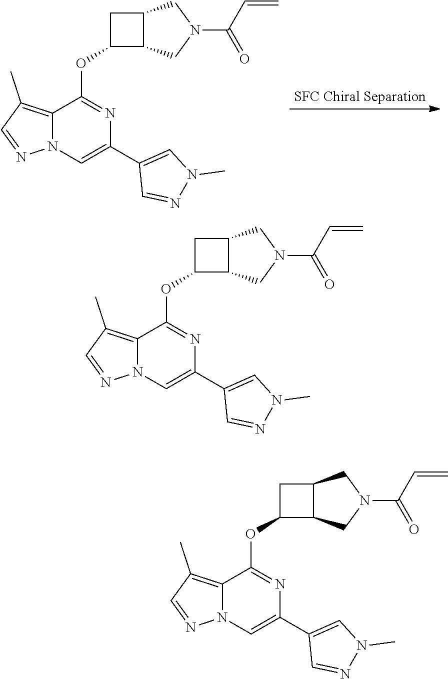

rac-1-((1S,5R,6R)-6-((3-methyl-6-(1-methyl-1H-pyrazol-4-yl)pyrazolo[1,5-a]pyrazin-4-yl)oxy)-3-azabicyclo[3.2.0]heptan-3-yl)prop-2-en-1-one (65 mg) was separated by chiral SFC purification (LUX Cellulose-2 LC 30×250 mm, 5 um, 40% MeOH in CO₂, flow rate: 100 mL/min, ABPR 120 bar, MBPR 40 psi, column temp 40° C.) then purified by chiral SFC a second time (CHIRALPAK AD-H 30×250 mm, 5 um, 40% MeOH in CO₂, flow rate: 100 mL/min, ABPR 120 bar, MBPR 40 psi, column temp 40° C.) to afford the first eluting peak (Example 197) (E1) (8 mg, 12%, Rt=2.81 min, 100% ee) and a second eluting peak (Example 198) (E2) (8 mg, 12%, Rt=3.20 min, 97.26% ee) as white solids.

### Examples 199 & 200: 1-((1R,5S,6R)-6-methyl-6-((6-(1-methyl-1H-pyrazol-4-yl)pyrazolo[1,5-a]pyrazin-4-yl)oxy)-3-azabicyclo[3.2.0]heptan-3-yl)prop-2-en-1-one and 1-((1S,5R,6S)-6-methyl-6-((6-(1-methyl-1H-pyrazol-4-yl)pyrazolo[1,5-a]pyrazin-4-yl)oxy)-3-azabicyclo[3.2.0]heptan-3-yl)prop-2-en-1-one

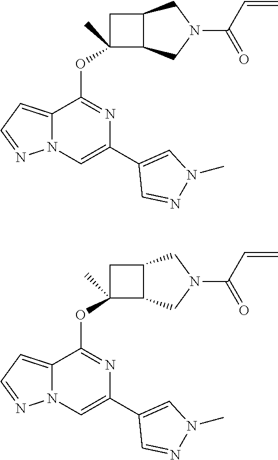 [SMILES: C=CC(=O)N1C[C@@H]2C[C@@](C)(Oc3nc(-c4cnn(C)c4)cn4nccc34)[C@@H]2C1 ; C=CC(=O)N1C[C@H]2C[C@](C)(Oc3nc(-c4cnn(C)c4)cn4nccc34)[C@H]2C1]

### 1. Synthesis of rac-(1R,5R,6R)-3-benzyl-6-methyl-2,4-dioxo-3-azabicyclo[3.2.0]heptan-6-yl acetate

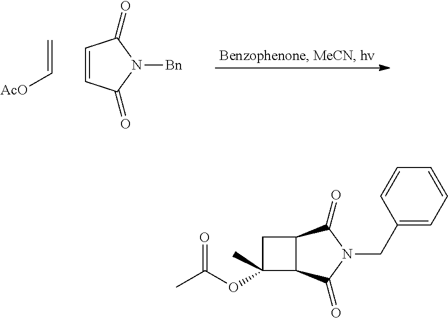

A mixture of N-benzylmaleimide (10 g, 53.4 mmol), isopropenyl acetate (6.00 g, 59.9 mmol) and benzophenone (500 mg, 2.74 mmol) in MeCN (350 mL) was stirred at RT under N₂ under a UV light for 3 days. The reaction was evaporated to dryness and purified by chromatography (heptane to EtOAc) to afford rac-(1R,5R,6R)-3-benzyl-6-methyl-2,4-dioxo-3-azabicyclo[3.2.0]heptan-6-yl acetate (2.2 g, 14%). ESI-MS (M+H)⁺: 288.1.

### 2. Synthesis of rac-(1R,5S,6R)-3-benzyl-6-methyl-3-azabicyclo[3.2.0]heptan-6-ol

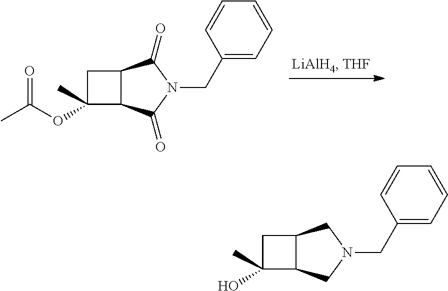 [SMILES: CC(=O)O[C@]1(C)C[C@H]2C(=O)N(Cc3ccccc3)C(=O)[C@H]21 ; C[C@@]1(O)C[C@H]2CN(Cc3ccccc3)C[C@H]21]

LiAlH₄ (2 M in THF, 1.32 mL) was added to an ice-cold solution of rac-(1R,5R,6R)-3-benzyl-6-methyl-2,4-dioxo-3-azabicyclo[3.2.0]heptan-6-yl acetate (200 mg, 0.696 mmol) in THF (3 mL) and the reaction stirred at 50° C. overnight. The reaction was cooled in an ice bath and water (2 mL) added slowly, followed by 30% NaOH (1 mL) resulting in a gel like solid to form. The mixture was allowed to settle and the organic layer collected. The remaining solids were rinsed with warm THF (3×10 mL). The combined organics were evaporated to dryness in vacuo to give a clear oil which was purified by chromatography (heptane to 3:1 EtOAc:EtOH with 2% diethylamine) to give rac-(1R,5S,6R)-3-benzyl-6-methyl-3-azabicyclo[3.2.0]heptan-6-ol as a clear oil (95 mg, 63%) which was used without further purification. ESI-MS (M+H)⁺: 218.1.

### 3. Synthesis of rac-4-(((1R,5S,6R)-3-benzyl-6-methyl-3-azabicyclo[3.2.0]heptan-6-yl)oxy)-6-(1-methyl-1H-pyrazol-4-yl)pyrazolo[1,5-a]pyrazine

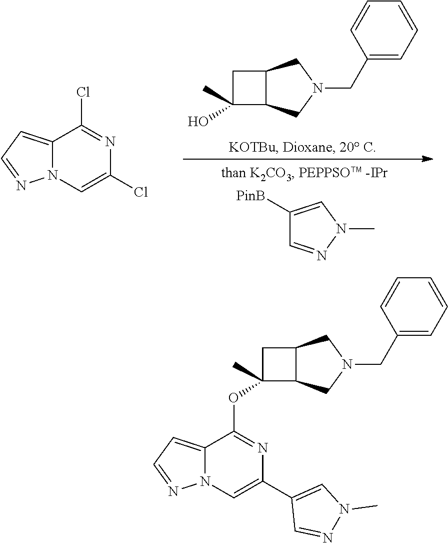

KOtBu (1 M THF, 437 μL) was added to a solution of rac-(1R,5S,6R)-3-benzyl-6-methyl-3-azabicyclo[3.2.0]heptan-6-ol (95 mg, 0.437 mmol) in THF (3 mL) and the resulting mixture was evaporated to dryness in vacuo. The resulting potassium salt was dissolved in THF (3 mL) and 4,6-dichloropyrazolo[1,5-a]pyrazine (85 mg, 0.452 mmol) added and the mixture was stirred for 15 mins at RT. The reaction was evaporated to dryness and to the residue was added 1-methyl-4-(4,4,5,5-tetramethyl-1,3,2-dioxaborolan-2-yl)pyrazole (225 mg, 1.08 mmol), potassium carbonate (200 mg, 1.45 mmol), PEPPSI™-IPr (15 mg, 0.022 mmol), dioxane (3 mL), and water (1 mL) and the mixture heated at 100° C. for 25 mins. The reaction mixture was diluted with 10 mL water and extracted with EtOAc (3×10 mL). The combined organics were evaporated to dryness and purified by chromatography (heptane to EtOAc) to give rac-4-(((1R,5S,6R)-3-benzyl-6-methyl-3-azabicyclo[3.2.0]heptan-6-yl)oxy)-6-(1-methyl-1H-pyrazol-4-yl)pyrazolo[1,5-a]pyrazine (160 mg, 88%). ESI-MS (M+H)⁺: 415.2.

### 4. Synthesis of rac-1-((1S,5R,6S)-6-methyl-6-((6-(1-methyl-1H-pyrazol-4-yl)pyrazolo[1,5-a]pyrazin-4-yl)oxy)-3-azabicyclo[3.2.0]heptan-3-yl)prop-2-en-1-one

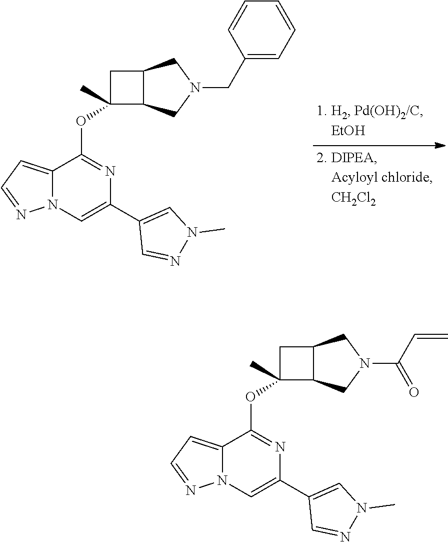 [SMILES: Cn1cc(-c2cn3nccc3c(O[C@]3(C)C[C@H]4CN(Cc5ccccc5)C[C@H]43)n2)cn1 ; C=CC(=O)N1C[C@@H]2C[C@@](C)(Oc3nc(-c4cnn(C)c4)cn4nccc34)[C@@H]2C1]

Part 1: Pd(OH)₂/C (150 mg, 0.213 mmol, 20% purity) was added to a solution of rac-4-(((1R,5S,6R)-3-benzyl-6-methyl-3-azabicyclo[3.2.0]heptan-6-yl)oxy)-6-(1-methyl-1H-pyrazol-4-yl)pyrazolo[1,5-a]pyrazine (160 mg, 0.386 mmol) in EtOH (5 mL). The mixture was purged with a balloon of H₂ for 5 mins and then stirred at RT overnight. The reaction mixture was filtered through a pad of Celite® and the pad washed with 3:1 EtOAc:EtOH (50 mL). The combined organics were evaporated to dryness in vacuo and the residue purified by chromatography (heptane to 3:1 EtOAc:EtOH with 2% diethylamine) to give rac-1-((1S,5R,6S)-6-methyl-6-((6-(1-methyl-1H-pyrazol-4-yl)pyrazolo[1,5-a]pyrazin-4-yl)oxy)-3-azabicyclo[3.2.0]heptan-3-yl)prop-2-en-1-one (40 mg, 32%) which was used without further purification. ESI-MS (M+H)⁺: 325.1.

Part 2: Acryloyl chloride (11.2 mg, 0.123 mmol) was added to a solution of rac-1-((1S,5R,6S)-6-methyl-6-((6-(1-methyl-1H-pyrazol-4-yl)pyrazolo[1,5-a]pyrazin-4-yl)oxy)-3-azabicyclo[3.2.0]heptan-3-yl)prop-2-en-1-one (40 mg, 0.123 mmol) in DCM (5 mL) and DIPEA (47.8 mg, 0.37 mmol) at −20° C. and the mixture stirred for 10 mins. The reaction was purified by chromatography (heptane to 3:1 EtOAc:EtOH) to give rac-1-((1S,5R,6S)-6-methyl-6-((6-(1-methyl-1H-pyrazol-4-yl)pyrazolo[1,5-a]pyrazin-4-yl)oxy)-3-azabicyclo[3.2.0]heptan-3-yl)prop-2-en-1-one as a colorless oil, (35 mg, 75%). ESI-MS (M+H)⁺: 379.2.

### 5. Isolation of 1-((1R,5S,6R)-6-methyl-6-((6-(I-methyl-1H-pyrazol-4-yl)pyrazolo[1,5-a]pyrazin-4-yl)oxy)-3-azabicyclo[3.2.0]heptan-3-yl)prop-2-en-1-one and 1-((1S,5R,6S)-6-methyl-6-((6-(1-methyl-1H-pyrazol-4-yl)pyrazolo[1,5-a]pyrazin-4-yl)oxy)-3-azabicyclo[3.2.0]heptan-3-yl)prop-2-en-1-one

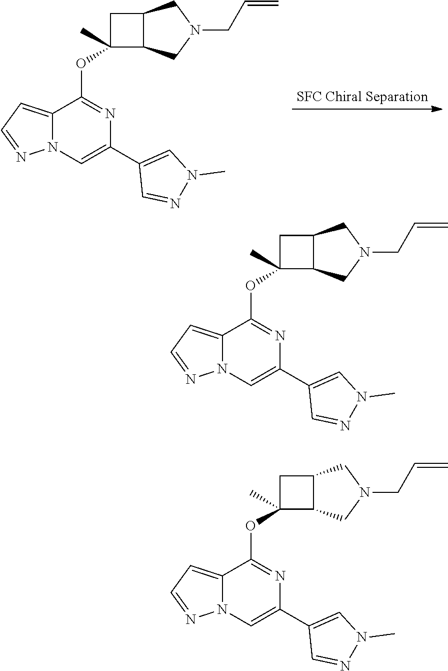

rac-1-((1S,5R,6S)-6-Methyl-6-((6-(1-methyl-1H-pyrazol-4-yl)pyrazolo[1,5-a]pyrazin-4-yl)oxy)-3-azabicyclo[3.2.0]heptan-3-yl)prop-2-en-1-one (35 mg) was separated by chiral SFC purification (LUX Cellulose-2 LC 30×250 mm, 5 μm, 40% MeOH in CO₂, flow rate: 100 mL/min, ABPR 120 bar, MBPR 40 psi, column temp 40° C.) to afford the two products as a first eluting peak (Example 199) (E1) (9 mg, 25%, 3.33 min, 95.6% ee) and a second eluting peak (Example 200) (E2) (9 mg, 25%, 3.93 min, 95.95% ee).

### Example 201: (E)-4,4,4-trifluoro-N-methyl-N-((1s,3s)-3-methyl-3-((7-(1-methyl-1H-pyrazol-4-yl)imidazo[1,2-c]pyrimidin-5-yl)oxy)cyclobutyl)but-2-enamide

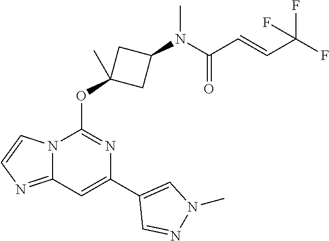 [SMILES: Cn1cc(-c2cc3nccn3c(O[C@]3(C)C[C@H](N(C)C(=O)/C=C/C(F)(F)F)C3)n2)cn1]

### 1. Synthesis of tert-butyl ((1s,3s)-3-methyl-3-((7-(1-methyl-1H-pyrazol-4-yl)imidazo[1,2-c]pyrimidin-5-yl)oxy)cyclobutyl)carbamate

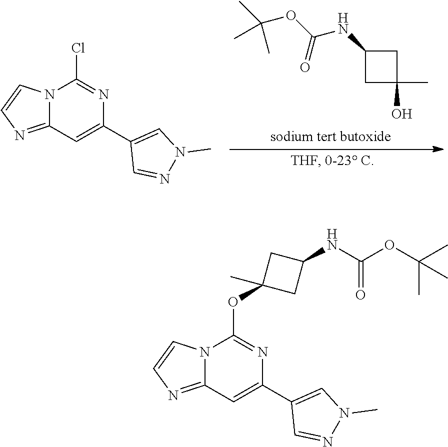 [SMILES: Cn1cc(-c2cc3nccn3c(Cl)n2)cn1 ; CC(C)(C)OC(=O)N[C@H]1C[C@@](C)(O)C1 ; Cn1cc(-c2cc3nccn3c(O[C@]3(C)C[C@H](NC(=O)OC(C)(C)C)C3)n2)cn1]

NaOtBu (348 mg, 3.62 mmol) was added in portions to an ice cold solution of tert-butyl ((1r,3r)-3-hydroxy-3-methylcyclobutyl)carbamate (501 mg, 2.49 mmol) in anhydrous THF (5 mL) and the mixture stirred for 15 mins before 5-chloro-7-(1-methylpyrazol-4-yl)imidazo[1,2-c]pyrimidine (469 mg, 2.01 mmol) was added. The reaction mixture was warmed to RT and stirred for 45 mins. The reaction was quenched with water and extracted with EtOAc (x3). The combined organics were dried (Na₂SO₄) and evaporated to dryness in vacuo. The residue was purified by silica gel chromatography (10-70% 3:1 EtOAc:EtOH in heptane) to afford tert-butyl ((1s,3s)-3-methyl-3-((7-(1-methyl-1H-pyrazol-4-yl)imidazo[1,2-c]pyrimidin-5-yl)oxy)cyclobutyl)carbamate as a white solid (796 mg, 100%). ¹H NMR (400 MHz, DMSO-d₆) δ=8.21 (s, 1H), 8.02 (s, 1H), 7.68 (dd, J=0.8, 1.5 Hz, 1H), 7.50 (d, J=1.5 Hz, 1H), 7.44 (s, 1H), 7.22 (br d, J=7.8 Hz, 1H), 3.91-3.88 (m, 3H), 3.88-3.77 (m, 1H), 2.85-2.77 (m, 2H), 2.47-2.41 (m, 2H), 1.75 (s, 3H), 1.37 (s, 9H).

### 2. Synthesis of tert-butyl methyl((1s,3s)-3-methyl-3-((7-(1-methyl-1H-pyrazol-4-yl)imidazo[1,2-c]pyrimidin-5-yl)oxy)cyclobutyl)carbamate

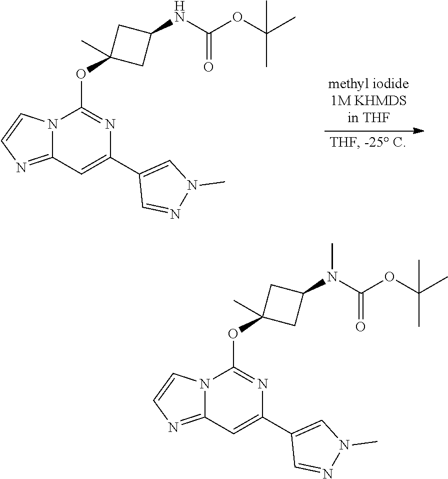 [SMILES: Cn1cc(-c2cc3nccn3c(O[C@]3(C)C[C@H](NC(=O)OC(C)(C)C)C3)n2)cn1 ; Cn1cc(-c2cc3nccn3c(O[C@]3(C)C[C@H](N(C)C(=O)OC(C)(C)C)C3)n2)cn1]

KHMDS in THF (1 M, 7 mL) was added dropwise to a solution of tert-butyl methyl((1s,3s)-3-methyl-3-((7-(1-methyl-1H-pyrazol-4-yl)imidazo[1,2-c]pyrimidin-5-yl)oxy)cyclobutyl)carbamate (796 mg, 2.00 mmol) in anhydrous THF (10 mL) at −25° C. under N₂. The reaction mixture was stirred at −25° C. for 10 mins before Mel (0.3 mL, 4.82 mmol) was added dropwise and the mixture stirred at RT for 2 h. The reaction was quenched with water and extracted with EtOAc (×3). The combined organics were dried (Na₂SO₄) and evaporated to dryness in vacuo. The residue was purified by silica gel chromatography (15-85% 3:1 EtOAc:EtOH in heptane) to afford tert-butyl methyl((1s,3s)-3-methyl-3-((7-(1-methyl-1H-pyrazol-4-yl)imidazo[1,2-c]pyrimidin-5-yl)oxy)cyclobutyl)carbamate as a white solid (168 mg, 20%). ¹H NMR (500 MHz, DMSO-d₆) δ=8.22 (s, 1H), 8.02 (s, 1H), 7.68 (s, 1H), 7.50 (d, J=1.2 Hz, 1H), 7.44 (s, 1H), 4.52-3.98 (m, 1H), 3.89 (s, 4H), 2.79-2.72 (m, 3H), 2.72-2.62 (m, 3H), 1.79 (s, 3H), 1.40 (s, 9H).

### 3. Synthesis of (1s,3s)-N,3-dimethyl-3-((7-(1-methyl-1H-pyrazol-4-yl)imidazo[1,2-c]pyrimidin-5-yl)oxy)cyclobutan-1-amine trifluoroacetate

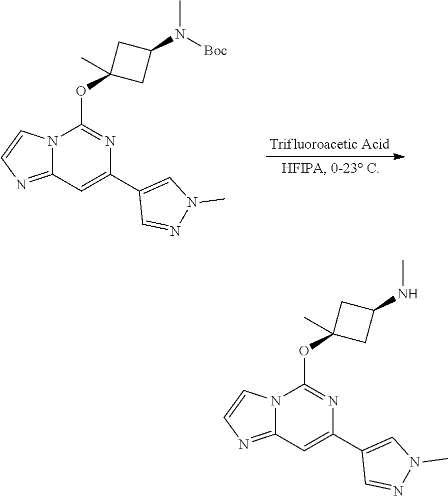 [SMILES: Cn1cc(-c2cc3nccn3c(O[C@]3(C)C[C@H](N(C)C(=O)OC(C)(C)C)C3)n2)cn1 ; CN[C@H]1C[C@@](C)(Oc2nc(-c3cnn(C)c3)cc3nccn23)C1]

TFA (0.1 mL, 1.31 mmol) was added to tert-butyl methyl((1s,3s)-3-methyl-3-((7-(1-methyl-1H-pyrazol-4-yl)imidazo[1,2-c]pyrimidin-5-yl)oxy)cyclobutyl)carbamate (168 mg, 0.407 mmol) in IPA (2.8 mL) at 0° C. and the mixture stirred at RT for 45 mins. The reaction mixture was evaporated to dryness under reduced pressure to afford (1s,3s)-N,3-dimethyl-3-((7-(1-methyl-1H-pyrazol-4-yl)imidazo[1,2-c]pyrimidin-5-yl)oxy)cyclobutan-1-amine trifluoroacetate as a colourless film (173 mg, 100%). ESI-MS (M+H)⁺: 313.1.

### 4. Synthesis of (E)-4,4,4-trifluoro-N-methyl-N-((1s,3s)-3-methyl-3-((7-(1-methyl-1H-pyrazol-4-yl)imidazo[1,2-c]pyrimidin-5-yl)oxy)cyclobutyl)but-2-enamide

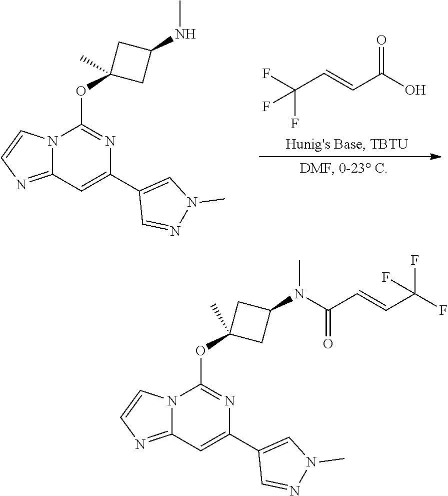 [SMILES: CN[C@H]1C[C@@](C)(Oc2nc(-c3cnn(C)c3)cc3nccn23)C1 ; O=C(O)/C=C/C(F)(F)F ; Cn1cc(-c2cc3nccn3c(O[C@]3(C)C[C@H](N(C)C(=O)/C=C/C(F)(F)F)C3)n2)cn1]

DIPEA (0.6 mL, 3.44 mmol) and TBTU (461 mg, 1.44 mmol) were added to an ice-cold mixture of (1s,3s)-N,3-dimethyl-3-((7-(1-methyl-1H-pyrazol-4-yl)imidazo[1,2-c]pyrimidin-5-yl)oxy)cyclobutan-1-amine trifluoroacetate (173 mg, 0.46 mmol) and (E)-4,4,4-trifluorobut-2-enoic acid (160 mg, 1.14 mmol) in dry DMF (2 mL) and the reaction stirred for 18 h at RT. The reaction mixture was diluted with sat aq NaHCO₃ solution and extracted with DCM (×3). The combined organics were washed with brine, dried (MgSO₄) and evaporated to dryness in vacuo. The residue was purified by prep-HPLC (Waters XSelect CSH C18, 100×50 mm, 5 mm; 5-75% MeCN/H₂O+0.2% NH₄OH) to afford (E)-4,4,4-trifluoro-N-methyl-N-((1s,3s)-3-methyl-3-((7-(1-methyl-1H-pyrazol-4-yl)imidazo[1,2-c]pyrimidin-5-yl)oxy)cyclobutyl)but-2-enamide (66.4 mg, 28%). ESI-MS (M+H)⁺: 435.1. ¹H NMR (500 MHz, DMSO-d₆) δ=8.22 (d, J=10.4 Hz, 1H), 8.02 (d, J=6.1 Hz, 1H), 7.71 (s, 1H), 7.51 (s, 1H), 7.50-7.24 (m, 2H), 6.81-6.70 (m, 1H), 4.73-4.48 (m, 1H), 3.89 (d, J=1.8 Hz, 3H), 3.03-2.90 (m, 3H), 2.85-2.80 (m, 2H), 2.80-2.72 (m, 2H), 1.84 (d, J=4.9 Hz, 3H).

### Example 202: rac-1-((3R,4S)-4-((3-chloro-6-(1-methyl-1H-pyrazol-4-yl)pyrazolo[1,5-a]pyrazin-4-yl)oxy)-3-fluoroazepan-1-yl)prop-2-en-1-one

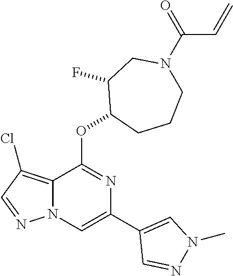

### 1. Synthesis of rac-tert-butyl (3S,4S)-3-fluoro-4-[6-(1-methylpyrazol-4-yl)pyrazolo[1,5-a]pyrazin-4-yl]oxy-azepane-1-carboxylate and rac-tert-butyl (3R,4S)-3-fluoro-4-[6-(1-methylpyrazol-4-yl)pyrazolo[1,5-a]pyrazin-4-yl]oxy-azepane-1-carboxylate

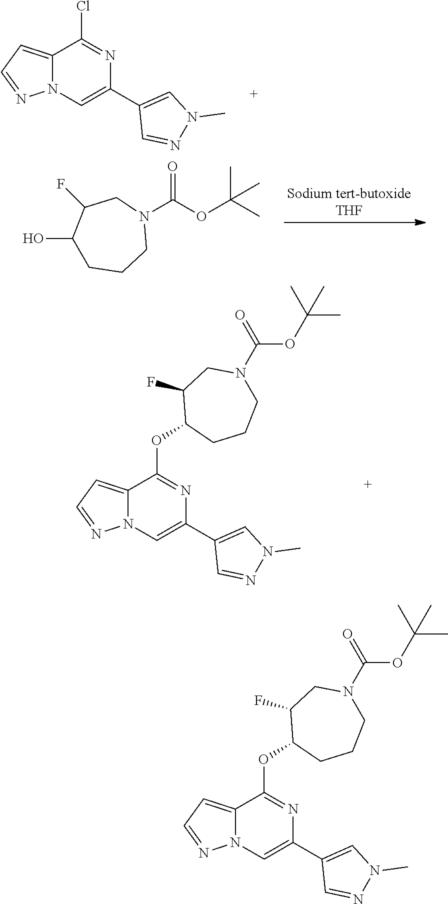

NaOtBu (2M, 1.5 mL) was added to tert-butyl 3-fluoro-4-hydroxyazepane-1-carboxylate (406 mg, 1.74 mmol) in anhydrous THF (10 mL) at 0° C. and the mixture stirred for 10 mins before 4-chloro-6-(1-methylpyrazol-4-yl)pyrazolo[1,5-a]pyrazine (390 mg, 1.67 mmol) was added and the resulting mixture stirred at RT for 18 h. The reaction mixture was evaporated to dryness and the residue dissolved in EtOAc, washed with brine, dried (Na₂SO₄) and evaporated to dryness in vacuo. The residue was purified by column chromatography (SiO₂, 20-65% EtOAc/heptane) to afford the title compounds as colourless oils.

Peak 1; rac-tert-butyl (3S,4S)-3-fluoro-4-[6-(1-methylpyrazol-4-yl)pyrazolo[1,5-a]pyrazin-4-yl]oxy-azepane-1-carboxylate (200 mg, 28%). ESI-MS (M+H)⁺: 431.2.

Peak 2; rac-tert-butyl (3R,4S)-3-fluoro-4-[6-(1-methylpyrazol-4-yl)pyrazolo[1,5-a]pyrazin-4-yl]oxy-azepane-1-carboxylate (250 mg, 35%). ESI-MS (M+H)⁺: 431.2.

### 2. Synthesis of rac-tert-butyl (3R,4S)-4-((3-chloro-6-(1-methyl-1H-pyrazol-4-yl)pyrazolo[1,5-a]pyrazin-4-yl)oxy)-3-fluoro-azepane-1-carboxylate

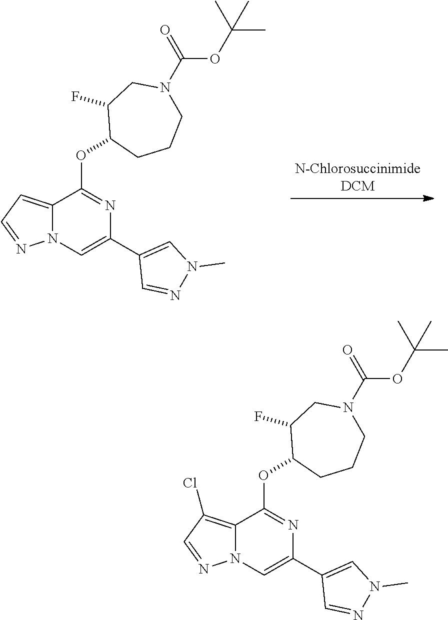

N-Chlorosuccinimide (96.2 mg, 0.72 mmol) was added to an ice-cold solution of rac-tert-butyl (3R,4S)-3-fluoro-4-[6-(1-methylpyrazol-4-yl)pyrazolo[1,5-a]pyrazin-4-yl]oxy-azepane-1-carboxylate (62 mg, 0.144 mmol) in anhydrous DCM (2 mL) and the resulting mixture stirred at RT for 24 h. The reaction was evaporated to dryness in vacuo and the residue was purified by column chromatography (10-30% (3:1 EtOAc:EtOH) in Heptane) to afford rac-tert-butyl (3R,4S)-4-((3-chloro-6-(1-methyl-1H-pyrazol-4-yl)pyrazolo[1,5-a]pyrazin-4-yl)oxy)-3-fluoro-azepane-1-carboxylate (40 mg, 60%). ESI-MS (M+H)⁺: 465.2.

### 3. Synthesis of rac-3-chloro-4-(((3R,4S)-3-fluoroazepan-4-yl)oxy)-6-(1-methyl-1H-pyrazol-4-yl)pyrazolo[1,5-a]pyrazine trifluoroacetate

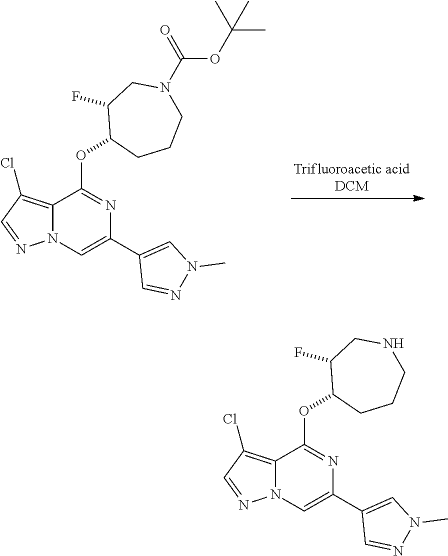

TFA (74.1 mg, 0.650 mmol) was added to an ice-cold solution of rac-tert-butyl (3R,4S)-4-((3-chloro-6-(1-methyl-1H-pyrazol-4-yl)pyrazolo[1,5-a]pyrazin-4-yl)oxy)-3-fluoro-azepane-1-carboxylate (40 mg, 0.086 mmol) in DCM (5 mL) and the mixture was stirred at RT for 2.5 h. The reaction was evaporated to dryness to afford rac-3-chloro-4-(((3R,4S)-3-fluoroazepan-4-yl)oxy)-6-(1-methyl-1H-pyrazol-4-yl)pyrazolo[1,5-a]pyrazine trifluoroacetate as a pale yellow oil (31 mg, 99%) which was used without further purification. ESI-MS (M+H)⁺: 365.2.

### 4. Synthesis of rac-1-((3R,4S)-4-((3-chloro-6-(1-methyl-1H-pyrazol-4-yl)pyrazolo[1,5-a]pyrazin-4-yl)oxy)-3-fluoroazepan-1-yl)prop-2-en-1-one

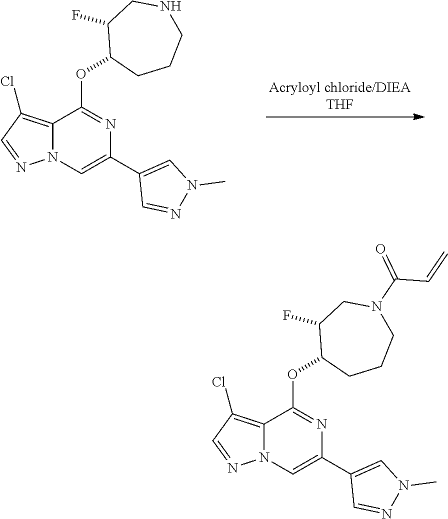

Acryloyl chloride (11.5 mg, 0.127 mmol) was added to a solution of rac-3-chloro-4-(((3R,4S)-3-fluoroazepan-4-yl)oxy)-6-(1-methyl-1H-pyrazol-4-yl)pyrazolo[1,5-a]pyrazine (31 mg, 0.085 mmol) and DIPEA (274 mg, 2.12 mmol) in anhydrous THF (5 mL) and the reaction stirred at RT for 3 mins. The reaction was quenched with sat. aq. NaHCO₃ and the biphasic mixture loaded directly onto a silica gel column eluting with 15-75% (3:1 EtOAc/EtOH) in hexanes to afford rac-1-((3R,4S)-4-((3-chloro-6-(1-methyl-1H-pyrazol-4-yl)pyrazolo[1,5-a]pyrazin-4-yl)oxy)-3-fluoroazepan-1-yl)prop-2-en-1-one as a white solid (5 mg, 14%). ESI-MS (M+H)⁺: 419.2. ¹H NMR (400 MHz, CDCl₃) δ 8.18 (d, J=6.02 Hz, 1H), 8.05 (m, 2H), 6.87 (dd, J=8.78, 2.26 Hz, 1H), 6.60-6.79 (m, 1H), 6.44 (m, 1H), 5.78 (m, 1H), 5.61-5.71 (m, 1H), 4.94-5.16 (m, 1H), 4.03 (s, 3H), 3.56-3.87 (m, 3H), 3.36 (dt, J=13.68, 6.96, 1H), 2.19-2.29 (m, 1H), 1.98-2.06 (m, 2H), 1.88 (dt, J=6.71, 3.29, 1H).

### Example 203: rac-1-((3S,4S)-4-((3-chloro-6-(1-methyl-1H-pyrazol-4-yl)pyrazolo[1,5-a]pyrazin-4-yl)oxy)-3-fluorocycloheptyl)prop-2-en-1-one

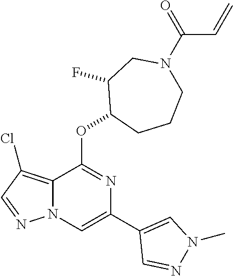

The title compound was prepared from rac-tert-butyl (3S,4S)-3-fluoro-4-[6-(1-methylpyrazol-4-yl)pyrazolo[1,5-a]pyrazin-4-yl]oxy-azepane-1-carboxylate using an analogous 3-step method as described for Example 202. ESI-MS (M+H)⁺: 419.2. 1H NMR (400 MHz, CDCl₃) δ 8.14 (s, 1H), 8.12 (s, 1H), 8.04 (m, 1H), 6.84 (m, 1H), 6.51-6.66 (m, 1H), 6.41 (m, 1H), 5.73 (m, 1H), 5.53-5.67 (m, 1H), 4.72-4.84 (m, 1H), 3.97 (s, 3H), 3.51-3.77 (m, 3H), 3.36 (m, 1H), 2.15-2.24 (m, 1H), 2.00-2.09 (m, 1H), 1.88-1.93 (m, 2H).

### Examples 204, 205, 206 & 207: Synthesis of 1-((5S,7S)-7-((6-(1-methyl-1H-pyrazol-4-yl)pyrazolo[1,5-a]pyrazin-4-yl)oxy)-2-azaspiro[4.4]nonan-2-yl)prop-2-en-1-one, 1-((5S,7R)-7-((6-(1-methyl-1H-pyrazol-4-yl)pyrazolo[1,5-a]pyrazin-4-yl)oxy)-2-azaspiro[4.4]nonan-2-yl)prop-2-en-1-one, 1-((5R,7S)-7-((6-(1-methyl-1H-pyrazol-4-yl)pyrazolo[1,5-a]pyrazin-4-yl)oxy)-2-azaspiro[4.4]nonan-2-yl)prop-2-en-1-one and 1-((5R,7R)-7-((6-(1-methyl-1H-pyrazol-4-yl)pyrazolo[1,5-a]pyrazin-4-yl)oxy)-2-azaspiro[4.4]nonan-2-yl)prop-2-en-1-one

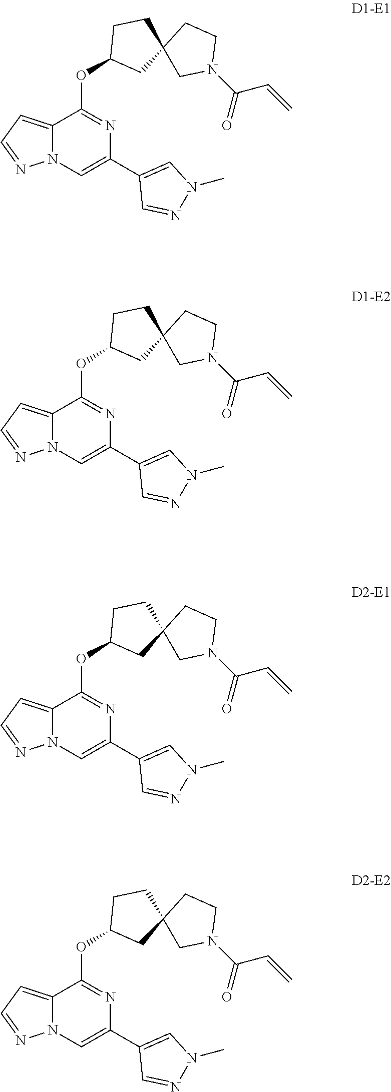 [SMILES: C=CC(=O)N1CC[C@]2(CC[C@H](Oc3nc(-c4cnn(C)c4)cn4nccc34)C2)C1 ; C=CC(=O)N1CC[C@]2(CC[C@@H](Oc3nc(-c4cnn(C)c4)cn4nccc34)C2)C1 ; C=CC(=O)N1CC[C@@]2(CC[C@H](Oc3nc(-c4cnn(C)c4)cn4nccc34)C2)C1 ; C=CC(=O)N1CC[C@@]2(CC[C@@H](Oc3nc(-c4cnn(C)c4)cn4nccc34)C2)C1]

Relative and absolute stereochemistry of isomers was arbitrarily assigned

### 1. Synthesis of rac-tert-butyl (5S)-7-((6-(1-methyl-1H-pyrazol-4-yl)pyrazolo[1,5-a]pyrazin-4-yl)oxy)-2-azaspiro[4.4]nonane-2-carboxylate

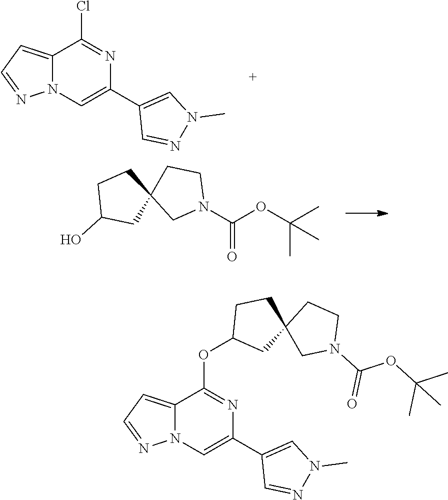

NaOtBu (0.94 mL, 2M in THF) was added to an ice-cold solution of rac-tert-butyl (5R)-7-hydroxy-2-azaspiro[4.4]nonane-2-carboxylate (250 mg, 1.04 mmol) in THF (12 mL) and the mixture stirred for 30 mins before 4-chloro-6-(1-methylpyrazol-4-yl)pyrazolo[1,5-a]pyrazine (220 mg, 0.942 mmol) was added and stirring continued for 3.5 h at RT. The reaction was quenched with water and extracted with EtOAc (×3). The combined organics were dried (Na₂SO₄) and evaporated to dryness in vacuo. The residue was purified by column chromatography (SiO₂, 0-35% (3:1 EtOAc/EtOH) in heptane) to afford rac-tert-butyl (5S)-7-((6-(1-methyl-1H-pyrazol-4-yl)pyrazolo[1,5-a]pyrazin-4-yl)oxy)-2-azaspiro[4.4]nonane-2-carboxylate. ESI-MS (M+H)⁺: 439.3.

### 2. Synthesis of rac-4-(((5S)-2-azaspiro[4.4]nonan-7-yl)oxy)-6-(1-methyl-1H-pyrazol-4-yl)pyrazolo[1,5-a]pyrazine hydrochloride

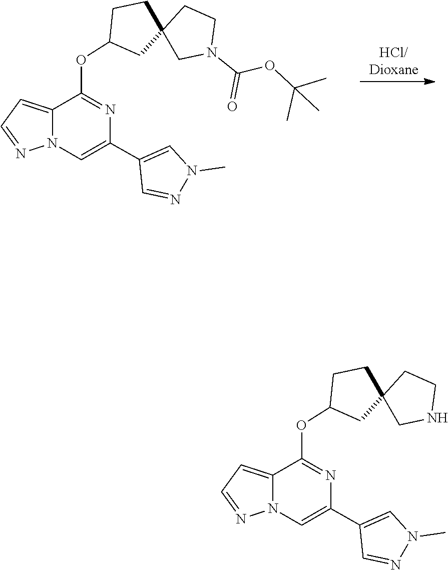

HCl (4M in dioxane, 3.1 mL) was added to rac-tert-butyl (5S)-7-((6-(1-methyl-1H-pyrazol-4-yl)pyrazolo[1,5-a]pyrazin-4-yl)oxy)-2-azaspiro[4.4]nonane-2-carboxylate (360 mg, 0.821 mmol) and the mixture stirred at RT for 1 h. The reaction was evaporated to dryness to afford rac-4-(((5S)-2-azaspiro[4.4]nonan-7-yl)oxy)-6-(1-methyl-1H-pyrazol-4-yl)pyrazolo[1,5-a]pyrazine hydrochloride as a pale yellow oil (280 mg, 100%). ESI-MS (M+H)⁺: 339.2.

### 3. Synthesis of 1-((5S,7S)-7-((6-(1-methyl-1H-pyrazol-4-yl)pyrazolo[1,5-a]pyrazin-4-yl)oxy)-2-azaspiro[4.4]nonan-2-yl)prop-2-en-1-one, 1-((5S,7R)-7-((6-(1-methyl-1H-pyrazol-4-yl)pyrazolo[1,5-a]pyrazin-4-yl)oxy)-2-azaspiro[4.4]nonan-2-yl)prop-2-en-1-one, 1-((5R,7S)-7-((6-(1-methyl-1H-pyrazol-4-yl)pyrazolo[1,5-a]pyrazin-4-yl)oxy)-2-azaspiro[4.4]nonan-2-yl)prop-2-en-1-one and 1-((5R,7R)-7-((6-(1-methyl-1H-pyrazol-4-yl)pyrazolo[1,5-a]pyrazin-4-yl)oxy)-2-azaspiro[4.4]nonan-2-yl)prop-2-en-1-one

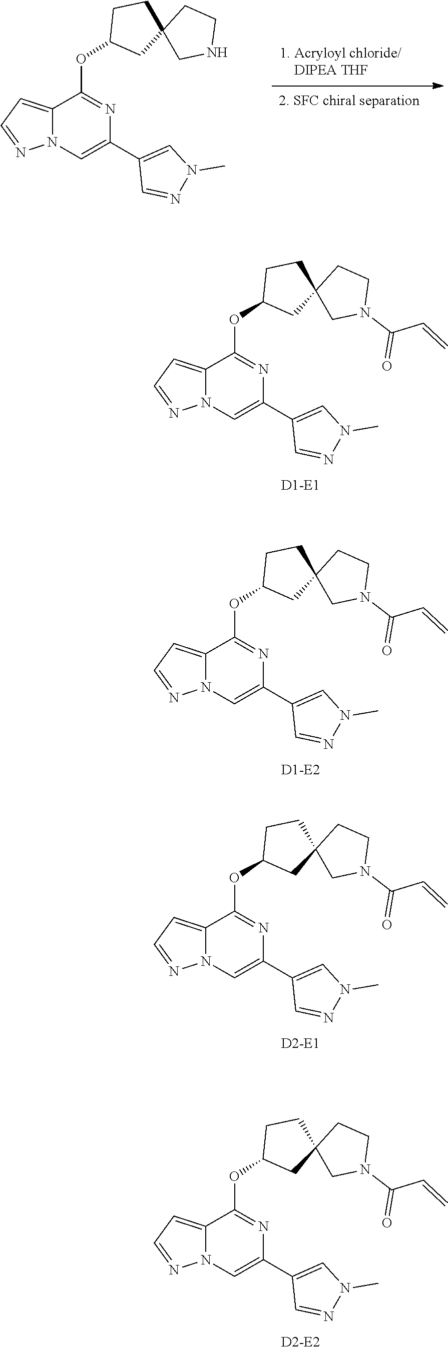 [SMILES: Cn1cc(-c2cn3nccc3c(O[C@@H]3CC[C@]4(CCNC4)C3)n2)cn1 ; C=CC(=O)N1CC[C@]2(CC[C@H](Oc3nc(-c4cnn(C)c4)cn4nccc34)C2)C1 ; C=CC(=O)N1CC[C@]2(CC[C@@H](Oc3nc(-c4cnn(C)c4)cn4nccc34)C2)C1 ; C=CC(=O)N1CC[C@@]2(CC[C@H](Oc3nc(-c4cnn(C)c4)cn4nccc34)C2)C1 ; C=CC(=O)N1CC[C@@]2(CC[C@@H](Oc3nc(-c4cnn(C)c4)cn4nccc34)C2)C1]

DIPEA (1.07 g, 8.30 mmol) was added to an ice-cold solution of rac-4-(((5S)-2-azaspiro[4.4]nonan-7-yl)oxy)-6-(1-methyl-1H-pyrazol-4-yl)pyrazolo[1,5-a]pyrazine hydrochloride (140 mg, 0.41 mmol) in dry THF (5 mL) and the mixture stirred for 5 mins before acryloyl chloride (56.2 mg, 1.24 mmol) was added and the reaction mixture stirred at RT for 3 mins. The reaction was quenched with sat. aq. NaHCO₃ and the biphasic mixture loaded directly onto a silica gel column eluting with 15-75% (3:1 EtOAc/EtOH) in hexanes to afford rac-tert-butyl (5S)-7-((6-(1-methyl-1H-pyrazol-4-yl)pyrazolo[1,5-a]pyrazin-4-yl)oxy)-2-azaspiro[4.4]nonane-2-carboxylate (225 mg, 69%). This material was separated into two diastereomers (D1 and D2) by SFC (AD-H column with 30% MeOH w/No Modifier in CO2 (flow rate: 100 mL/min, ABPR 120 bar, MBPR 40 psi, column temp 40 deg C).

Diastereomer 1 (D1) was separated by chiral SFC chromatography (Chiralpak 1A 30×250 mm, 5 um AD-H column. Method: 35% iPrOH w/No Modifier in CO2 (flow rate: 100 mL/min, ABPR 120 bar, MBPR 40 psi, column temp 40° C.)) to afford:

### Example 204, Peak 1 first eluting isomer (D1-E1); (10.6 mg, 100% ee). ESI-MS (M+H)⁺: 393.9, Rf=4.79 min

### Example 205, Peak 2, second eluting isomer (D1-E2); (9.6 mg, 100% ee). ESI-MS (M+H)⁺: 393.9, Rf=4.85 min. ¹H NMR (400 MHz, CDCl₃) δ 8.22 (s, 1H), 7.79-7.90 (m, 3H), 6.72 (m, 1H), 6.38-6.49 (m, 2H), 5.69-5.77 (m, 2H), 4.27 (s, 3H), 3.66 (m, 2H), 3.48-3.52 (m, 2H), 2.25-2.33 (m, 2H), 1.96-2.09 (m, 5H), 1.73-1.82 (m, 1H)

Diastereomer 2 was separated by chiral SFC chromatography (Chiralpak 1A 30×250 mm, 5 um AD-H column. Method: 40% EtOH w/No Modifier in CO2 (flow rate: 100 mL/min, ABPR 120 bar, MBPR 40 psi, column temp 40° C.)) to afford:

### Example 206, Peak 1, first eluting isomer (D2-E1); (43.7 mg, 99.6% ee). ESI-MS (M+H)⁺: 393.9, Rf=4.09 min

### Example 207, Peak 2, second eluting isomer (D2-E1); (43.5 mg, 100% ee). ESI-MS (M+H)⁺: 393.9, Rf=5.03 min

Relative and absolute stereochemistry of isomers was arbitrarily assigned

### Example 208: 1-(2-((6-(1-methyl-1H-pyrazol-4-yl)pyrazolo[1,5-a]pyrazin-4-yl)oxy)-7-azaspiro[3.5]nonan-7-yl)prop-2-en-1-one

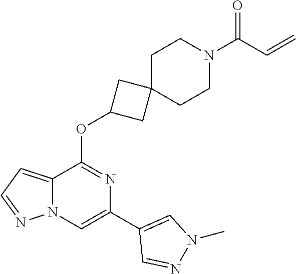 [SMILES: C=CC(=O)N1CCC2(CC1)CC(Oc1nc(-c3cnn(C)c3)cn3nccc13)C2]

### 1. Synthesis of tert-butyl 2-((6-(1-methyl-1H-pyrazol-4-yl)pyrazolo[1,5-a]pyrazin-4-yl)oxy)-7-azaspiro[3.5]nonane-7-carboxylate

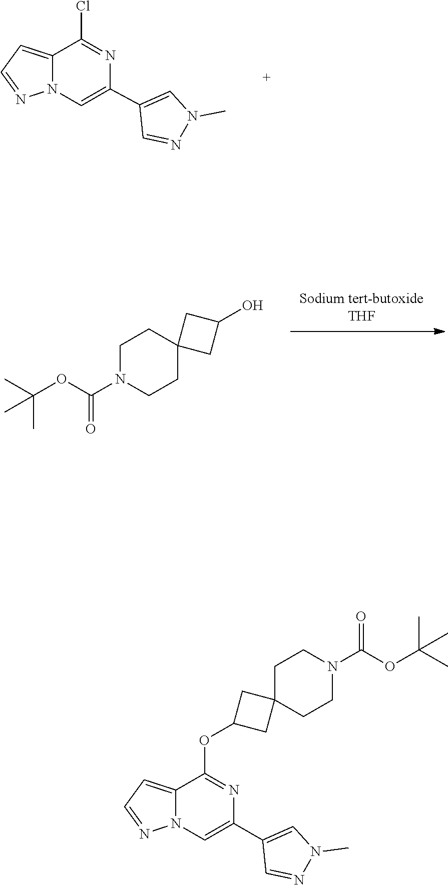 [SMILES: Cn1cc(-c2cn3nccc3c(Cl)n2)cn1 ; CC(C)(C)OC(=O)N1CCC2(CC1)CC(O)C2 ; Cn1cc(-c2cn3nccc3c(OC3CC4(CCN(C(=O)OC(C)(C)C)CC4)C3)n2)cn1]

NaO^{t}Bu (2M in THF, 0.565 mL) was added to an ice-cold solution of tert-butyl 2-hydroxy-7-azaspiro[3.5]nonane-7-carboxylate (150 mg, 0.622 mmol) in dry THF (10 mL) and the mixture stirred for 15 mins before 4-chloro-6-(1-methylpyrazol-4-yl)pyrazolo[1,5-a]pyrazine (132 mg, 0.565 mmol) and the resulting mixture stirred at RT for 3.5 h. The reaction was diluted with water and extracted with EtOAc (×3). The combined organics were dried (Na₂SO₄) and evaporated to dryness in vacuo. The residue was purified by column chromatography (SiO2, 0-35% (3:1 EtOAc:EtOH) in heptane) to afford tert-butyl 2-((6-(1-methyl-1H-pyrazol-4-yl)pyrazolo[1,5-a]pyrazin-4-yl)oxy)-7-azaspiro[3.5]nonane-7-carboxylate as a white solid (170 mg, 69%). ESI-MS (M+H)⁺: 439.3.

### 2. Synthesis of 4-((7-azaspiro[3.5]nonan-2-yl)oxy)-6-(1-methyl-1H-pyrazol-4-yl)pyrazolo[1,5-a]pyrazine

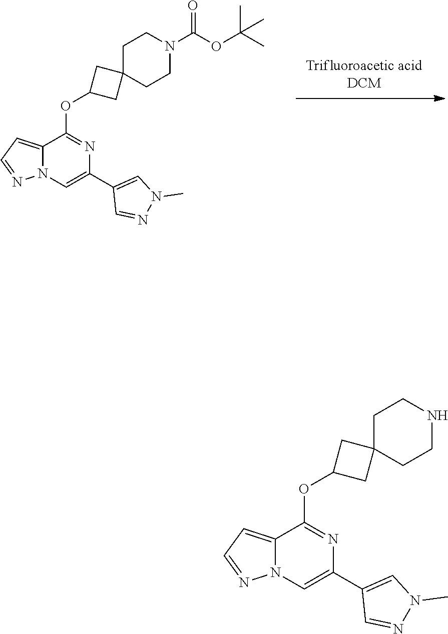 [SMILES: Cn1cc(-c2cn3nccc3c(OC3CC4(CCN(C(=O)OC(C)(C)C)CC4)C3)n2)cn1 ; Cn1cc(-c2cn3nccc3c(OC3CC4(CCNCC4)C3)n2)cn1]

TFA (0.224 mL, 2.93 mmol) was added to an ice-cold solution of tert-butyl 2-((6-(1-methyl-1H-pyrazol-4-yl)pyrazolo[1,5-a]pyrazin-4-yl)oxy)-7-azaspiro[3.5]nonane-7-carboxylate (170 mg, 0.387 mmol) in dry DCM (1 mL). The reaction mixture was allowed to warm to RT and stirred for 2.5 h. The reaction mixture was evaporated to dryness in vacuo to afford 4-(7-azaspiro[3.5]nonan-2-yloxy)-6-(1-methylpyrazol-4-yl)pyrazolo[1,5-a]pyrazine as a yellow film (131 mg, 100%) which was used without additional purification. ESI-MS (M+H)⁺: 339.2.

### 3. Synthesis of 1-(2-((6-(1-methyl-1H-pyrazol-4-yl)pyrazolo[1,5-a]pyrazin-4-yl)oxy)-7-azaspiro[3.5]nonan-7-yl)prop-2-en-1-one

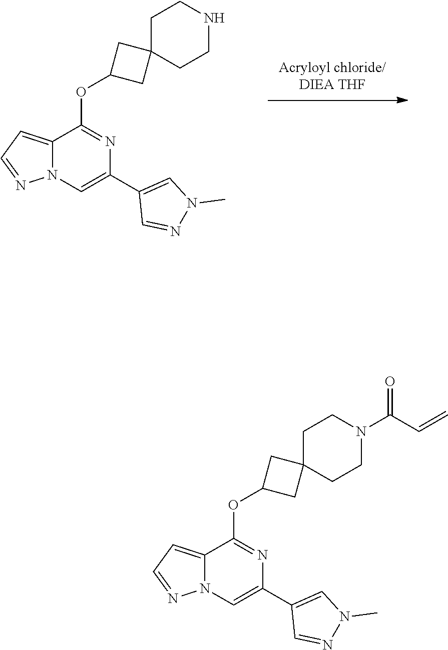 [SMILES: Cn1cc(-c2cn3nccc3c(OC3CC4(CCNCC4)C3)n2)cn1 ; C=CC(=O)N1CCC2(CC1)CC(Oc1nc(-c3cnn(C)c3)cn3nccc13)C2]

An ice cold solution of 4-((7-azaspiro[3.5]nonan-2-yl)oxy)-6-(1-methyl-1H-pyrazol-4-yl)pyrazolo[1,5-a]pyrazine (145 mg, 0.428 mmol) and DIPEA (1.11 g, 8.57 mmol) in THF was stirred at RT for 5 mins before acryloyl chloride (58.2 mg, 0.643 mmol) was added. The resulting mixture was stirred at RT for 3 mins and quenched with saturated aqueous NaHCO₃ solution. The resulting biphasic mixture was by column chromatography (SiO₂, (15-75% (3:1 EtOAc:EtOH) in hexanes) to afford 1-(2-((6-(1-methyl-1H-pyrazol-4-yl)pyrazolo[1,5-a]pyrazin-4-yl)oxy)-7-azaspiro[3.5]nonan-7-yl)prop-2-en-1-one as a white solid (110 mg, 65%). ESI-MS (M+H)⁺: 393.2. ¹H NMR (400 MHz, CDCl₃) δ 8.19 (m, 1H), 7.85-7.88 9M, 2H), 7.75 (M, 1H), 6.74 (br s, 1H), 6.58-6.60 (m, 1H), 6.26-6.28 (m, 1H), 5.66-5.68 (br d, J=9.79, 1H), 5.44-5.57 (m, 1H), 3.96 (m, 3H), 3.69 (m, 1H), 3.59 (m, 1H), 3.54 (m, 1H), 3.46 (br s, 1H), 2.57 (br s, 2H), 2.08 (m, 2H), 1.68-1.74 (m, 4H).

### Examples 209 & 210: rac-((1S,3R,5S)-3-((6-(1-methyl-1H-pyrazol-4-yl)pyrazolo[1,5-a]pyrazin-4-yl)oxy)-6-azabicyclo[3.2.0]heptan-6-yl)prop-2-en-1-one and rac-1-((1S,3S,5S)-3-((6-(1-methyl-1H-pyrazol-4-yl)pyrazolo[1,5-a]pyrazin-4-yl)oxy)-6-azabicyclo[3.2.0]heptan-6-yl)prop-2-en-1-one

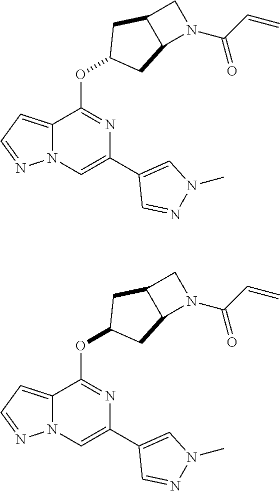

### 1. Synthesis of tert-butyl 3-((6-(1-methyl-1H-pyrazol-4-yl)pyrazolo[1,5-a]pyrazin-4-yl)oxy)-6-azabicyclo[3.2.0]heptane-6-carboxylate

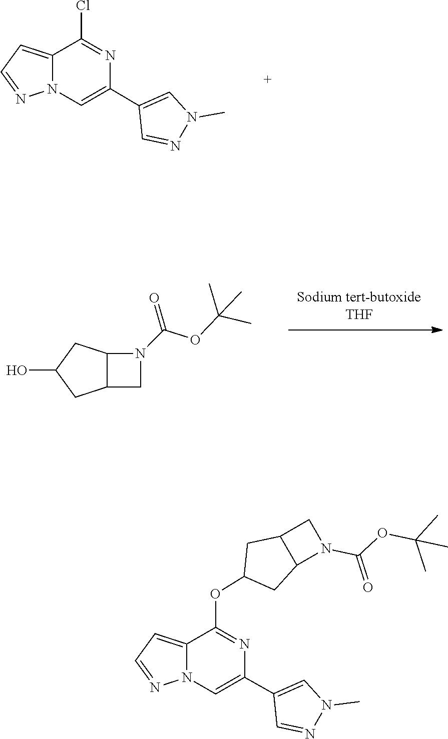 [SMILES: Cn1cc(-c2cn3nccc3c(Cl)n2)cn1 ; CC(C)(C)OC(=O)N1CC2CC(O)CC21 ; Cn1cc(-c2cn3nccc3c(OC3CC4CN(C(=O)OC(C)(C)C)C4C3)n2)cn1]

NaOtBu (1M in THF, 2.13 mL) was added to an ice-cold solution tert-butyl 3-hydroxy-6-azabicyclo[3.2.0]heptane-6-carboxylate (250 mg, 1.07 mmol) in dry THF (5 mL) and the mixture stirred for 45 mins before 4-chloro-6-(1-methylpyrazol-4-yl)pyrazolo[1,5-a]pyrazine was added and the resulting mixture was stirred at RT for 3.5 h. The reaction was diluted with water and extracted with EtOAc (x3). The combined organics were dried (Na₂SO₄) and evaporated to dryness in vacuo. The residue was purified by column chromatography (SiO₂, 0-35% (3:1 EtOAc:EtOH) in heptane) to afford rac-tert-butyl 3-((6-(1-methyl-1H-pyrazol-4-yl)pyrazolo[1,5-a]pyrazin-4-yl)oxy)-6-azabicyclo[3.2.0]heptane-6-carboxylate as a yellow foam (150 mg, 35%). ESI-MS (M+H)⁺: 411.2.

### 2. Synthesis of 4-((6-azabicyclo[3.2.0]heptan-3-yl)oxy)-6-(1-methyl-1H-pyrazol-4-yl)pyrazolo[1,5-a]pyrazine trifluoroacetate

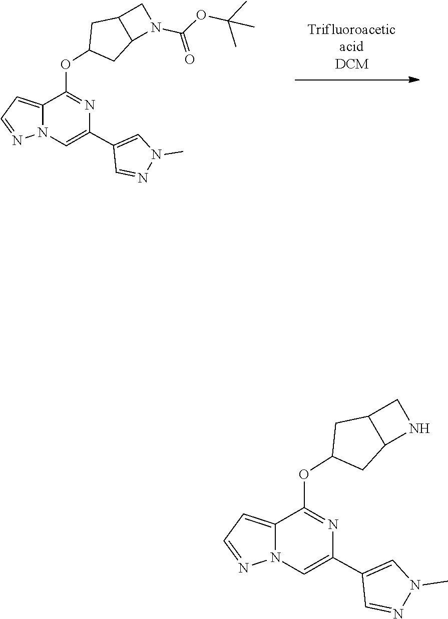 [SMILES: Cn1cc(-c2cn3nccc3c(OC3CC4CN(C(=O)OC(C)(C)C)C4C3)n2)cn1 ; Cn1cc(-c2cn3nccc3c(OC3CC4CNC4C3)n2)cn1]

TFA (315 mg, 2.76 mmol) was added to an ice-cold solution of 4-((6-azabicyclo[3.2.0]heptan-3-yl)oxy)-6-(1-methyl-1H-pyrazol-4-yl)pyrazolo[1,5-a]pyrazine (150 mg, 0.365 mmol) in dry DCM (5 mL) and the resulting mixture allowed to warm to RT and stirred for 2.5 h. The reaction mixture was evaporated to dryness to afford 4-((6-azabicyclo[3.2.0]heptan-3-yl)oxy)-6-(1-methyl-1H-pyrazol-4-yl)pyrazolo[1,5-a]pyrazine trifluoroacetate as a yellow oil (113 mg, 100%) which was used without additional purification. ESI-MS (M+H)⁺: 311.1.

### 3. Synthesis of rac-((1S,3R,5S)-3-((6-(1-methyl-1H-pyrazol-4-yl)pyrazolo[1,5-a]pyrazin-4-yl)oxy)-6-azabicyclo[3.2.0]heptan-6-yl)prop-2-en-1-one and rac-1-((1S,3S,5S)-3-((6-(1-methyl-1H-pyrazol-4-yl)pyrazolo[1,5-a]pyrazin-4-yl)oxy)-6-azabicyclo[3.2.0]heptan-6-yl)prop-2-en-1-one

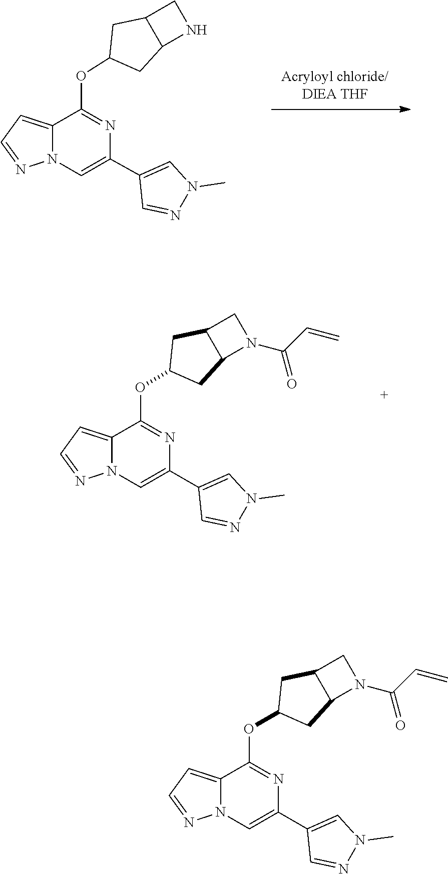 [SMILES: Cn1cc(-c2cn3nccc3c(OC3CC4CNC4C3)n2)cn1 ; C=CC(=O)N1C[C@@H]2C[C@@H](Oc3nc(-c4cnn(C)c4)cn4nccc34)C[C@@H]21 ; C=CC(=O)N1C[C@@H]2C[C@H](Oc3nc(-c4cnn(C)c4)cn4nccc34)C[C@@H]21]

An ice cold solution of 4-((6-azabicyclo[3.2.0]heptan-3-yl]oxy]-6-(1-methylpyrazol-4-yl)pyrazolo[1,5-a]pyrazine (67 mg, 0.216 mmol) and DIPEA (557 mg, 4.32 mmol) in THF (5 mL) was stirred at RT for 5 mins before acryloyl chloride (29.3 mg, 0.324 mmol) was added. The resulting mixture was stirred at RT for 3 mins and quenched with saturated aqueous NaHCO₃ solution. The resulting biphasic mixture was purified by column chromatography (SiO₂, (15-75% (3:1 EtOAc:EtOH) in hexanes) to afford the 2 diastereomers as white solids.

### Example 209 Peak 1, (10 mg, 13%); ESI-MS (M+H)⁺: 365.2; ¹H NMR (400 MHz, CDCl₃) δ 8.15 (d, J=6.27 Hz, 1H), 7.81 (d, J=2.26 Hz, 1H), 7.65-7.77 (m, 2H), 6.59-6.70 (m, 1H), 6.28-6.43 (m, 1H), 6.08-6.27 (m, 1H), 5.81-5.98 (m, 1H), 5.58-5.71 (m, 1H), 4.89 (q, J=6.27 Hz, 1H), 4.15-4.34 (m, 1H), 3.88 (d, J=9.79 Hz, 3H), 3.59-3.82 (m, 1H), 2.73-3.17 (m, 2H), 2.44 (td, J=13.05, 6.27 Hz, 1H), 1.64-2.01 (m, 2H)

### Example 210 Peak 2, (22 mg, 28%); ESI-MS (M+H)⁺: 365.2; ¹H NMR (400 MHz, CDCl₃) δ 8.12 (t, J=0.88 Hz, 1H), 7.73-7.81 (m, 2H), 7.71 (s, 1H), 6.43-6.78 (m, 1H) 6.08-6.28 (m, 2H), 5.95-6.08 (m, 1H), 5.50-5.63 (m, 1H), 4.89-5.00 (m, 1H), 4.06-4.33 (m, 1H), 3.83-4.02 (m, 4H), 3.02-3.15 (m, 1H), 2.50-2.93 (m, 1H), 2.24-2.36 (m, 1H), 1.88-2.11 (m, 2H)

### Example 211: N-((1s,3s)-3-((3-fluoro-6-(1-methyl-1H-pyrazol-4-yl)pyrazolo[1,5-a]pyrazin-4-yl)oxy)-3-methylcyclobutyl)-N-methylacrylamide

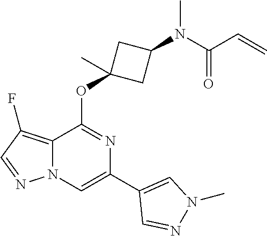 [SMILES: C=CC(=O)N(C)[C@H]1C[C@@](C)(Oc2nc(-c3cnn(C)c3)cn3ncc(F)c23)C1]

### 1. Synthesis of tert-butyl ((1s,3s)-3-((3-fluoro-6-(1-methyl-1H-pyrazol-4-yl)pyrazolo[1,5-a]pyrazin-4-yl)oxy)-3-methylcyclobutyl)(methyl)carbamate

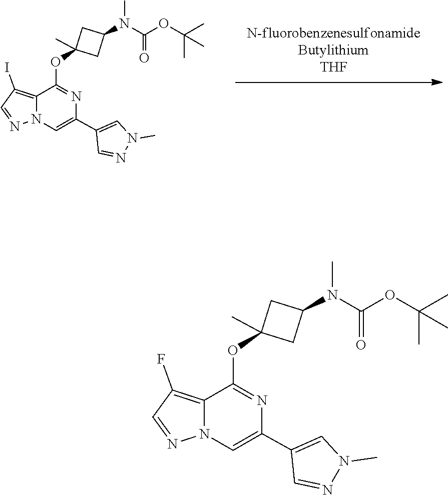 [SMILES: Cn1cc(-c2cn3ncc(I)c3c(O[C@]3(C)C[C@H](N(C)C(=O)OC(C)(C)C)C3)n2)cn1 ; Cn1cc(-c2cn3ncc(F)c3c(O[C@]3(C)C[C@H](N(C)C(=O)OC(C)(C)C)C3)n2)cn1]

BuLi (2.5 M, 0.156 mL) was added to a solution of tert-butyl ((1s,3s)-3-((3-iodo-6-(1-methyl-1H-pyrazol-4-yl)pyrazolo[1,5-a]pyrazin-4-yl)oxy)-3-methylcyclobutyl)(methyl)carbamate (150 mg, 0.278 mmol) in THF (3 mL) at −78° C. and the resulting mixture stirred for 20 mins before N-fluorobenzenesulfonimide (123 mg, 0.39 mmol) was added and stirring continued for a further 1 h. The reaction was quenched with aq. NH₄Cl and extracted with EtOAc. The combined organics were dried (Na₂SO₄) and evaporated to dryness in vacuo. The residue was purified by column chromatography (SiO₂, 0-25% (3:1 EtOAc:EtOH) in hexanes) to afford tert-butyl ((1s,3s)-3-((3-fluoro-6-(1-methyl-1H-pyrazol-4-yl)pyrazolo[1,5-a]pyrazin-4-yl)oxy)-3-methylcyclobutyl)(methyl)carbamate as an amorphous solid (50 mg, 42%). ESI-MS (M+H)⁺: 431.2.

### 2. Synthesis of (1s,3s)-3-((3-fluoro-6-(1-methyl-1H-pyrazol-4-yl)pyrazolo[1,5-a]pyrazin-4-yl)oxy)-N,3-dimethylcyclobutan-1-amine trifluoroacetate

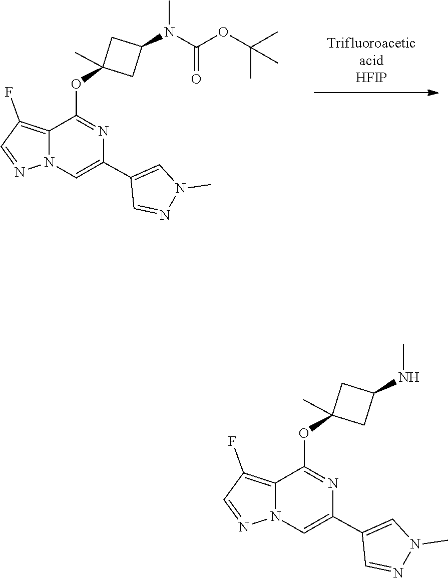 [SMILES: Cn1cc(-c2cn3ncc(F)c3c(O[C@]3(C)C[C@H](N(C)C(=O)OC(C)(C)C)C3)n2)cn1 ; CN[C@H]1C[C@@](C)(Oc2nc(-c3cnn(C)c3)cn3ncc(F)c23)C1]

TFA (90 mg, 0.79 mmol) was added to an ice-cold solution of tert-butyl ((1s,3s)-3-((3-fluoro-6-(1-methyl-1H-pyrazol-4-yl)pyrazolo[1,5-a]pyrazin-4-yl)oxy)-3-methylcyclobutyl)(methyl)carbamate (170 mg, 0.395 mmol) in HFIP (5 mL). The reaction mixture was stirred at RT for 90 mins and evaporated to dryness in vacuo to afford (1s,3s)-3-((3-fluoro-6-(1-methyl-1H-pyrazol-4-yl)pyrazolo[1,5-a]pyrazin-4-yl)oxy)-N,3-dimethylcyclobutan-1-amine trifluoroacetate which was used without further purification. ESI-MS (M+H)⁺: 331.1.

### 2. Synthesis of N-((1s,3s)-3-((3-fluoro-6-(1-methyl-1H-pyrazol-4-yl)pyrazolo[1,5-a]pyrazin-4-yl)oxy)-3-methylcyclobutyl)-N-methylacrylamide

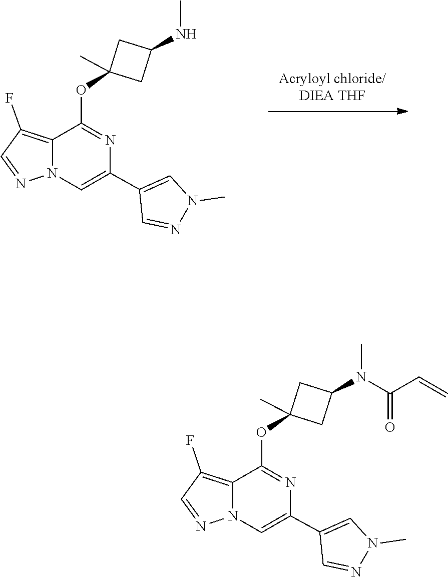 [SMILES: CN[C@H]1C[C@@](C)(Oc2nc(-c3cnn(C)c3)cn3ncc(F)c23)C1 ; C=CC(=O)N(C)[C@H]1C[C@@](C)(Oc2nc(-c3cnn(C)c3)cn3ncc(F)c23)C1]

An ice cold solution (1s,3s)-3-((3-fluoro-6-(1-methyl-1H-pyrazol-4-yl)pyrazolo[1,5-a]pyrazin-4-yl)oxy)-N,3-dimethylcyclobutan-1-amine trifluoroacetate (42.9 mg, 0.130 mmol) and DIPEA (419 mg, 3.24 mmol) in THF (5 mL) was stirred at RT for 5 mins before acryloyl chloride (17.6 mg, 0.195 mmol) was added. The resulting mixture was stirred at RT for 8 mins and quenched with saturated aqueous NaHCO₃ solution. The resulting biphasic mixture was by column chromatography (SiO₂, (15-75% (3:1 EtOAc:EtOH) in hexanes) to afford N—((1s,3s)-3-((3-fluoro-6-(1-methyl-1H-pyrazol-4-yl)pyrazolo[1,5-a]pyrazin-4-yl)oxy)-3-methylcyclobutyl)-N-methylacrylamide (10 mg, 20%). ESI-MS (M+H)⁺: 385.1. ¹H NMR (400 MHz, CDCl₃) δ 8.01 (s, 1H), 7.83-7.86 (m, 1H), 7.73-7.75 (m, 1H), 7.70-7.73 (m, 1H), 6.53-6.58 (m, 1H), 6.30-6.35 (m, 1H), 5.70-5.74 (m, 1H), 4.83-4.88 (m, 1H), 4.01 (s, 3H), 3.04 (s, 3H), 2.80-2.84 (m, 2H), 2.62-2.68 (m, 2H), 1.86 (s, 3H).

### Examples 212 & 213: rac-1-((2s,4r)-2-methyl-2-((6-(1-methyl-1H-pyrazol-4-yl)pyrazolo[1,5-a]pyrazin-4-yl)oxy)-6-azaspiro[3.4]octan-6-yl)prop-2-en-1-one and rac-1-((2r,4s)-2-methyl-2-((6-(1-methyl-1H-pyrazol-4-yl)pyrazolo[1,5-a]pyrazin-4-yl)oxy)-6-azaspiro[3.4]octan-6-yl)prop-2-en-1-one

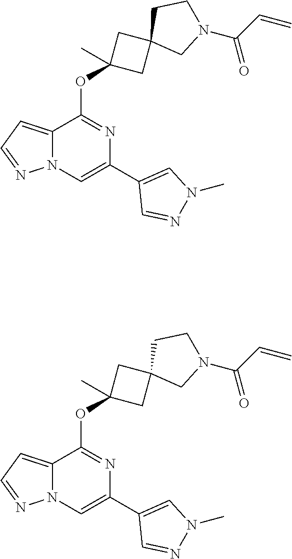 [SMILES: C=CC(=O)N1CC[C@]2(C1)C[C@](C)(Oc1nc(-c3cnn(C)c3)cn3nccc13)C2 ; C=CC(=O)N1CC[C@]2(C1)C[C@@](C)(Oc1nc(-c3cnn(C)c3)cn3nccc13)C2]

### 1. Synthesis of tert-butyl 2-methyl-2-[6-(1-methylpyrazol-4-yl)pyrazolo[1,5-a]pyrazin-4-yl]oxy-6-azaspiro[3.4]octane-6-carboxylate

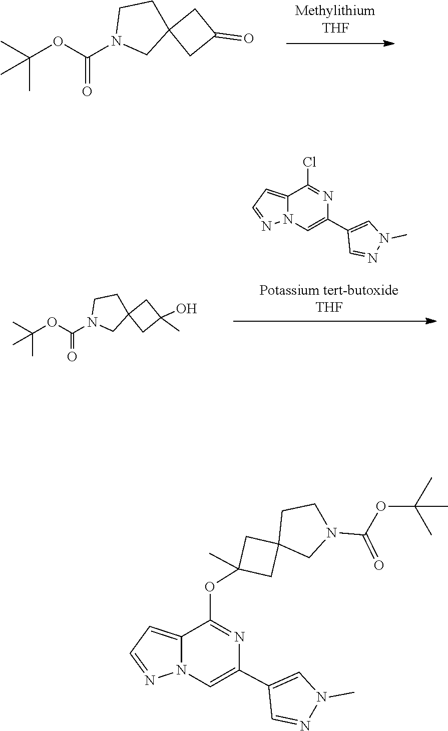 [SMILES: CC(C)(C)OC(=O)N1CCC2(CC(=O)C2)C1 ; Cn1cc(-c2cn3nccc3c(Cl)n2)cn1 ; CC1(O)CC2(CCN(C(=O)OC(C)(C)C)C2)C1 ; Cn1cc(-c2cn3nccc3c(OC3(C)CC4(CCN(C(=O)OC(C)(C)C)C4)C3)n2)cn1]

Methyl lithium (1.6 M, 2.77 mL) was added slowly to a solution of tert-butyl 2-oxo-6-azaspiro[3.4]octane-6-carboxylate (500 mg, 2.22 mmol) in THF (10 mL) at −78° C. and the reaction stirred at RT for 2 hrs. The reaction mixture was quenched with aq ammonium chloride, diluted with EtOAc (25 mL) and washed with sat. aq. NH₄Cl, water and brine. The combined organics were dried (Na₂SO₄) and evaporated to dryness in vacuo to afford tert-butyl 2-hydroxy-2-methyl-6-azaspiro[3.4]octane-6-carboxylate which was dissolved in dry THF (10 mL). To this ice-cold solution was added KOtBu (2M, 0.44 mL) and stirred for 10 mins before 4-chloro-3-iodo-6-(1-methylpyrazol-4-yl)pyrazolo[1,5-a]pyrazine (186 mg, 0.795 mmol) was added and stirring continued for 18 h at RT. The reaction mixture was evaporated to dryness and the residue taken up in EtOAc and washed with brine, dried (Na₂SO₄) and evaporated to dryness in vacuo. The residue was purified by column chromatography (SiO2, (0-35% (3:1 EtOAc:EtOH) in heptane) to afford tert-butyl 2-methyl-2-[6-(1-methylpyrazol-4-yl)pyrazolo[1,5-a]pyrazin-4-yl]oxy-6-azaspiro[3.4]octane-6-carboxylate. ESI-MS (M+H)⁺: 439.2.

### 2. Synthesis of 6-(1-methyl-1H-pyrazol-4-yl)-4-((2-methyl-6-azaspiro[3.4]octan-2-yl)oxy)pyrazolo[1,5-a]pyrazine trifluoroacetate

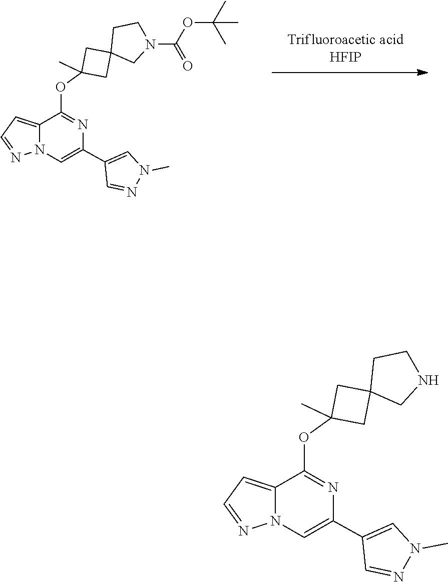 [SMILES: Cn1cc(-c2cn3nccc3c(OC3(C)CC4(CCN(C(=O)OC(C)(C)C)C4)C3)n2)cn1 ; Cn1cc(-c2cn3nccc3c(OC3(C)CC4(CCNC4)C3)n2)cn1]

TFA (865 mg, 7.59 mmol) was added to an ice-cold solution of tert-butyl 2-methyl-2-[6-(1-methylpyrazol-4-yl)pyrazolo[1,5-a]pyrazin-4-yl]oxy-6-azaspiro[3.4]octane-6-carboxylate (349 mg, 0.795 mmol) in HFIP (3 mL). The reaction mixture was stirred at RT for 2.5 h and evaporated to dryness in vacuo to afford 6-(1-methyl-1H-pyrazol-4-yl)-4-((2-methyl-6-azaspiro[3.4]octan-2-yl)oxy)pyrazolo[1,5-a]pyrazine trifluoroacetate as a pale yellow oil (245 mg, 91%) which was used without further purification. ESI-MS (M+H)⁺: 339.1.

### 3. Synthesis of 1-(2-methyl-2-((6-(1-methyl-1H-pyrazol-4-yl)pyrazolo[1,5-a]pyrazin-4-yl)oxy)-6-azaspiro[3.4]octan-6-yl)prop-2-en-1-one

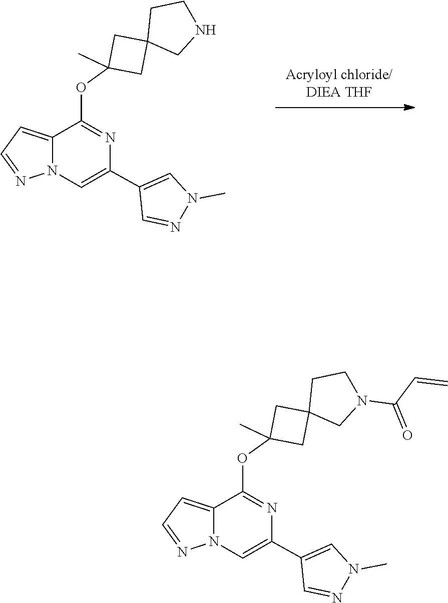 [SMILES: Cn1cc(-c2cn3nccc3c(OC3(C)CC4(CCNC4)C3)n2)cn1 ; C=CC(=O)N1CCC2(C1)CC(C)(Oc1nc(-c3cnn(C)c3)cn3nccc13)C2]

An ice cold solution 6-(1-methyl-1H-pyrazol-4-yl)-4-((2-methyl-6-azaspiro[3.4]octan-2-yl)oxy)pyrazolo[1,5-a]pyrazine trifluoroacetate (250 mg, 0.739 mmol) and DIPEA (2.29 g, 18.5 mmol) in THF (5 mL) was stirred at RT for 5 mins before acryloyl chloride (100 mg, 1.11 mmol) was added. The resulting mixture was stirred at RT for 24 h and quenched with saturated aqueous NaHCO₃ solution. The resulting biphasic mixture was purified by column chromatography (SiO₂, (15-75% (3:1 EtOAc:EtOH) in hexanes) to afford 1-(2-methyl-2-((6-(1-methyl-1H-pyrazol-4-yl)pyrazolo[1,5-a]pyrazin-4-yl)oxy)-6-azaspiro[3.4]octan-6-yl)prop-2-en-1-one. ESI-MS (M+H)⁺: 393.2. ¹H NMR (400 MHz, CDCl₃) δ 8.12-8.26 (m, 1H), 7.89 (t, J=2.38 Hz, 1H), 7.80-7.86 (m, 1H), 7.70-7.77 (m, 1H), 6.69-6.75 (m, 1H), 6.32-6.49 (m, 2H), 5.62-5.76 (m, 1H), 3.97-40.3 (m, 3H), 3.50-3.60 (m, 4H), 2.61-2.78 (m, 2H), 2.39-2.58 (m, 2H), 2.11-2.19 (m, 2H), 1.86-1.91 (m, 3H).

### 4. Preparation of rac-1-((2s,4r)-2-methyl-2-((6-(1-methyl-1H-pyrazol-4-yl)pyrazolo[1,5-a]pyrazin-4-yl)oxy)-6-azaspiro[3.4]octan-6-yl)prop-2-en-1-one and rac-1-((2r,4s)-2-methyl-2-((6-(1-methyl-1H-pyrazol-4-yl)pyrazolo[1,5-a]pyrazin-4-yl)oxy)-6-azaspiro[3.4]octan-6-yl)prop-2-en-1-one

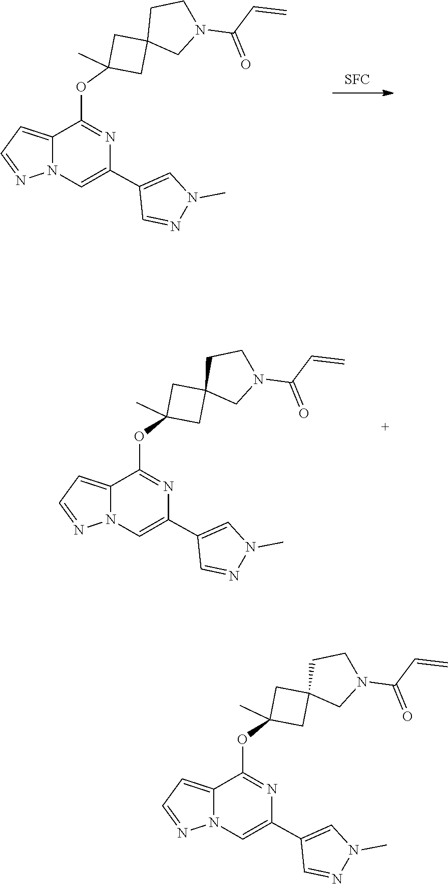 [SMILES: C=CC(=O)N1CCC2(C1)CC(C)(Oc1nc(-c3cnn(C)c3)cn3nccc13)C2 ; "SFC" ; C=CC(=O)N1CC[C@]2(C1)C[C@](C)(Oc1nc(-c3cnn(C)c3)cn3nccc13)C2 ; C=CC(=O)N1CC[C@]2(C1)C[C@@](C)(Oc1nc(-c3cnn(C)c3)cn3nccc13)C2]

1-(2-methyl-2-((6-(1-methyl-1H-pyrazol-4-yl)pyrazolo[1,5-a]pyrazin-4-yl)oxy)-6-azaspiro[3.4]octan-6-yl)prop-2-en-1-one (28.9 mg, 0.074 mmol) was purified by SFC (LUX Cellulose-4 LC 30x250 mm, 5 mm column. Method: 40% MeOH in CO₂ (flow rate: 100 mL/min, ABPR 120 bar, MBPR 40 psi, column temp 40° C.) to afford 2 diastereomers.

### Example 212 Peak 1, (Rf: 2.88 min, 98.88% ee). ESI-MS (M+H)⁺: 393.2

### Example 213 Peak 2, (Rf: 3.36 min, 96.12% ee.). ESI-MS (M+H)⁺: 393.2

### Example 214: 1-((1R,5S,6r)-6-(((6-(1-methyl-1H-pyrazol-4-yl)pyrazolo[1,5-a]pyrazin-4-yl)oxy)methyl)-3-azabicyclo[3.1.0]hexan-3-yl)prop-2-en-1-one

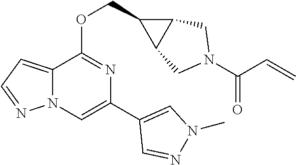 [SMILES: C=CC(=O)N1C[C@@H]2[C@@H](COc3nc(-c4cnn(C)c4)cn4nccc34)[C@@H]2C1]

### 1. Synthesis of 4-(((1R,5S,6r)-3-azabicyclo[3.1.0]hexan-6-yl)methoxy)-6-(1-methyl-1H-pyrazol-4-yl)pyrazolo[1,5-a]pyrazine

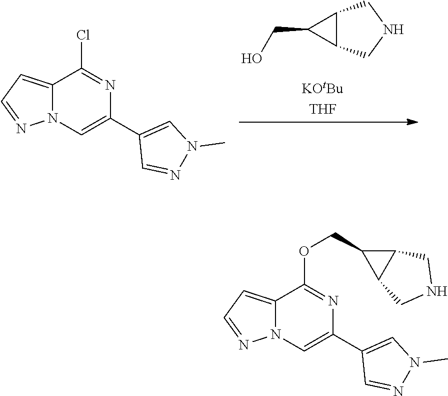 [SMILES: Cn1cc(-c2cn3nccc3c(Cl)n2)cn1 ; OC[C@@H]1[C@H]2CNC[C@@H]12 ; Cn1cc(-c2cn3nccc3c(OC[C@H]3[C@@H]4CNC[C@@H]43)n2)cn1]

To a solution of 4-chloro-6-(1-methyl-1H-pyrazol-4-yl)pyrazolo[1,5-a]pyrazine (100 mg, 428 μmol) and ((1R,5S,6r)-3-azabicyclo[3.1.0]hexan-6-yl)MeOH (61 mg, 535 μmol) in THF (4 mL) was added KOtBu(120 mg, 1.1 mmol) at RT. After 30 min the reaction mixture was diluted with EtOAc (25 mL) and washed with sat. aq. NH₄Cl (10 mL), water (10 mL), and brine (10 mL). The combined organics were dried (Na₂SO₄) and evaporated to dryness in vacuo to afford 4-(((1R,5S,6r)-3-azabicyclo[3.1.0]hexan-6-yl)methoxy)-6-(1-methyl-1H-pyrazol-4-yl)pyrazolo[1,5-a]pyrazine as a pale orange residue which was used in the next step without further purification (assuming 100% yield). LCMS m/z=311.0 (M+H)⁺.

### 2. Synthesis of 1-((1R,5S,6r)-6-(((6-(1-methyl-1H-pyrazol-4-yl)pyrazolo[1,5-a]pyrazin-4-yl)oxy)methyl)-3-azabicyclo[3.1.0]hexan-3-yl)prop-2-en-1-one

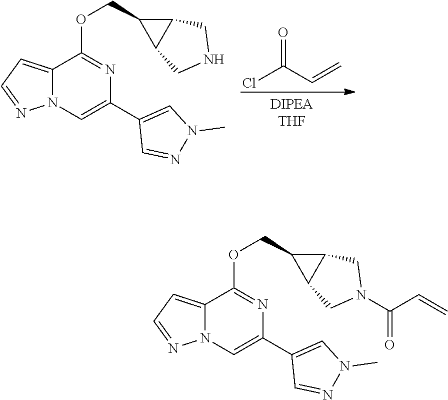

1-((1R,5S,6r)-6-(((6-(1-methyl-1H-pyrazol-4-yl)pyrazolo[1,5-a]pyrazin-4-yl)oxy)methyl)-3-azabicyclo[3.1.0]hexan-3-yl)prop-2-en-1-one was synthesized in the same way as Example 27 but starting from crude 4-(((1R,5S,6r)-3-azabicyclo[3.1.0]hexan-6-yl)methoxy)-6-(1-methyl-1H-pyrazol-4-yl)pyrazolo[1,5-a]pyrazine (132 mg, 425 μmol). The material was purified by column chromatography (24 g SiO₂, 0-100% EtOH:EtOAc (2% NH₄OH)1:3 in heptane) yielding the title compound (112 mg, 72% yield) as an off-white solid. LCMS m/z=387.1 (M+Na)⁺. ¹H NMR (500 MHz, acetonitrile-d₃) δ ppm 8.38 (s, 1H), 7.99 (s, 1H), 7.86-7.95 (m, 2H), 6.79 (d, J=3.05 Hz, 1H), 6.43-6.55 (m, 1H), 6.11-6.23 (m, 1H), 5.63 (dd, J=2.44, 10.38 Hz, 1H), 4.46-4.57 (m, 2H), 3.92 (s, 3H), 3.77-3.85 (m, 2H), 3.68 (dd, J=4.27, 10.38 Hz, 1H), 3.45 (dd, J=4.88, 12.21 Hz, 1H), 1.75-1.86 (m, 2H), 1.25 (tt, J=3.66, 7.02 Hz, 1H).

### Example 215: N-methyl-N-((cis)-3-((6-(1-methyl-1H-pyrazol-4-yl)pyrazolo[1,5-a]pyrazin-4-yl)oxy)cyclobutyl)acrylamide

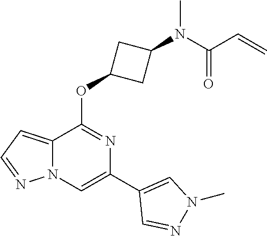 [SMILES: C=CC(=O)N(C)[C@H]1C[C@@H](Oc2nc(-c3cnn(C)c3)cn3nccc23)C1]

### 1. Synthesis of tert-butyl methyl((cis)-3-((6-(1-methyl-1H-pyrazol-4-yl)pyrazolo[1,5-a]pyrazin-4-yl)oxy)cyclobutyl)carbamate

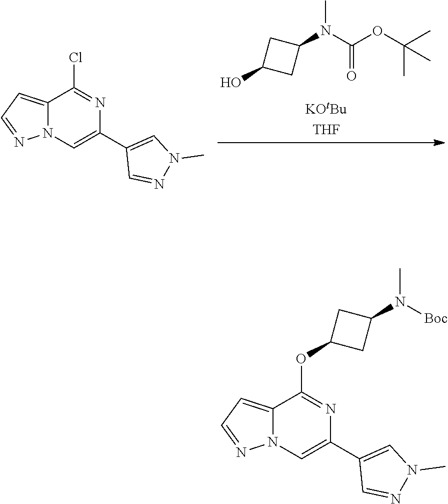 [SMILES: Cn1cc(-c2cn3nccc3c(Cl)n2)cn1 ; CN(C(=O)OC(C)(C)C)[C@H]1C[C@@H](O)C1 ; Cn1cc(-c2cn3nccc3c(O[C@H]3C[C@@H](N(C)C(=O)OC(C)(C)C)C3)n2)cn1]

tert-Butyl methyl((cis)-3-((6-(1-methyl-1H-pyrazol-4-yl)pyrazolo[1,5-a]pyrazin-4-yl)oxy)cyclobutyl)carbamate was synthesized in the same way as Example 214 but starting from tert-butyl ((cis)-3-hydroxycyclobutyl)(methyl)carbamate (750 mg, 3.7 mmol). The crude title compound was obtained as a pale orange residue (assuming 100% yield) which was used in the next without further purification. LCMS m/z=399.1 (M+H)⁺.

### 2. Synthesis of (cis)-N-methyl-3-((6-(1-methyl-1H-pyrazol-4-yl)pyrazolo[1,5-a]pyrazin-4-yl)oxy)cyclobutan-1-amine

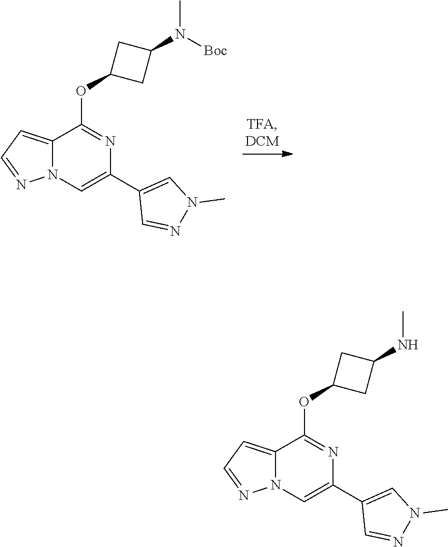 [SMILES: Cn1cc(-c2cn3nccc3c(O[C@H]3C[C@@H](N(C)C(=O)OC(C)(C)C)C3)n2)cn1 ; CN[C@H]1C[C@@H](Oc2nc(-c3cnn(C)c3)cn3nccc23)C1]

To a solution of crude tert-butyl methyl((cis)-3-((6-(1-methyl-1H-pyrazol-4-yl)pyrazolo[1,5-a]pyrazin-4-yl)oxy)cyclobutyl)carbamate (1.35 g, 3.4 mmol) in DCM (9 mL) was added TFA (3 mL) at RT and the reaction mixture stirred for 1 h. The reaction mixture was diluted with EtOAc (50 mL) followed by careful addition of sat. aq. NaHCO₃ (50 mL) with vigorous stirring. The resulting organic phase was separated and washed with water (10 mL), and brine (10 mL), separated, dried over Na₂SO₄, filtered, and concentrated under reduced pressure to afford (1s,3s)-N-methyl-3-((6-(1-methyl-1H-pyrazol-4-yl)pyrazolo[1,5-a]pyrazin-4-yl)oxy)cyclobutan-1-amine as an orange oil (assume 100%). LCMS m/z=299.0 (M+H)⁺.

### 3. Synthesis of N-methyl-N-((cis)-3-((6-(1-methyl-1H-pyrazol-4-yl)pyrazolo[1,5-a]pyrazin-4-yl)oxy)cyclobutyl)acrylamide

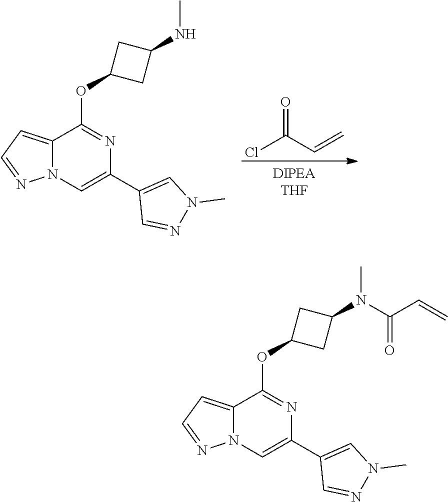

N-methyl-N-((cis)-3-((6-(1-methyl-1H-pyrazol-4-yl)pyrazolo[1,5-a]pyrazin-4-yl)oxy)cyclobutyl)acrylamide was synthesized in the same way as Example 27 but starting from crude (cis)-N-methyl-3-((6-(1-methyl-1H-pyrazol-4-yl)pyrazolo[1,5-a]pyrazin-4-yl)oxy)cyclobutan-1-amine (250 mg, 0.838 mmol). The material was purified by column chromatography (24 g SiO₂, 10-100% EtOH:EtOAc (2% NH₄OH)_{1:3} in heptane) yielding N-methyl-N-((cis)-3-((6-(1-methyl-1H-pyrazol-4-yl)pyrazolo[1,5-a]pyrazin-4-yl)oxy)cyclobutyl)acrylamide (209 mg, 71%) as an off-white solid. LCMS m/z=375.1 (M+Na)*. ¹H NMR (500 MHz, MeCN-d₃) δ ppm 8.32 (d, J=1.22 Hz, 1H), 7.94 (s, 1H), 7.83-7.89 (m, 2H), 6.60-6.76 (m, 2H), 6.15 (br d, J=14.65 Hz, 1H), 5.65 (dd, J=2.44, 10.38 Hz, 1H), 5.13 (br s, 1H), 4.25-4.84 (m, 1H), 3.88 (s, 3H), 2.80-3.09 (m, 5H), 2.30-2.55 (m, 2H)

### Example 216: N-methyl-N-((1-(((6-(1-methyl-1H-pyrazol-4-yl)pyrazolo[1,5-a]pyrazin-4-yl)oxy)methyl)cyclopropyl)methyl)acrylamide

### 1. Synthesis of N-methyl-1-(1-(((6-(1-methyl-1H-pyrazol-4-yl)pyrazolo[1,5-a]pyrazin-4-yl)oxy)methyl)cyclopropyl)methanamine

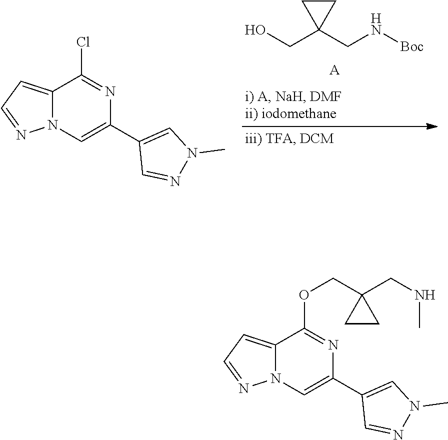

(1r,3r)-N-methyl-3-((6-(1-methyl-1H-pyrazol-4-yl)pyrazolo[1,5-a]pyrazin-4-yl)oxy)cyclobutan-1-amine was synthesized in the same way as Example 185 but starting from tert-butyl ((1-(hydroxymethyl)cyclopropyl)methyl)carbamate (47 mg, 0.235 mmol). The crude material was split in half and used in the next step without further purification (assuming 100% yield). LCMS m/z=313.1 (M+H)⁺.

### 2. Synthesis of N-methyl-N-((1-(((6-(1-methyl-1H-pyrazol-4-yl)pyrazolo[1,5-a]pyrazin-4-yl)oxy)methyl)cyclopropyl)methyl)acrylamide

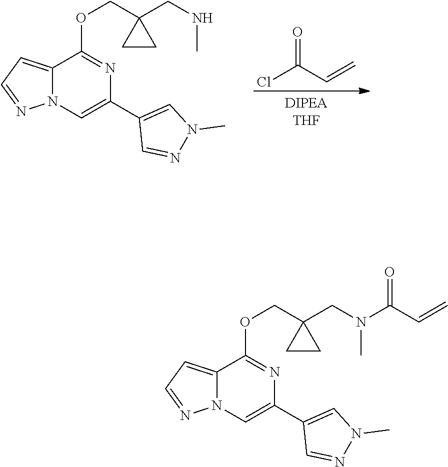

N-methyl-N-((1-(((6-(1-methyl-1H-pyrazol-4-yl)pyrazolo[1,5-a]pyrazin-4-yl)oxy)methyl)cyclopropyl)methyl)acrylamide was synthesized in the same way as Example 27 but starting from crude N-methyl-1-(1-(((6-(1-methyl-1H-pyrazol-4-yl)pyrazolo[1,5-a]pyrazin-4-yl)oxy)methyl)cyclopropyl)methanamine (33 mg, 0.107 mmol). The material was purified by column chromatography (24 g SiO2, 10-100% EtOH:EtOAc (2% NH₄OH)1:3 in heptane) yielding N-methyl-N-((1-(((6-(1-methyl-1H-pyrazol-4-yl)pyrazolo[1,5-a]pyrazin-4-yl)oxy)methyl)cyclopropyl)methyl)acrylamide (209 mg, 71% yield) as an off-white solid. LCMS m/z=389.1 (M+Na)⁺. ¹H NMR (500 MHz, acetonitrile-d₃) δ ppm 8.29-8.40 (m, 1H), 7.96 (s, 1H), 7.92 (dd, J=2.14, 12.51 Hz, 1H), 7.87 (s, 1H), 6.61-6.90 (m, 2H), 5.93-6.06 (m, 1H), 5.24-5.61 (m, 1H), 4.29-4.49 (m, 2H), 3.90 (s, 3H), 3.61 (s, 2H), 3.00-3.21 (m, 3H), 0.66-0.84 (m, 4H).

### Example 217: N-methyl-N-((1r,3r)-3-methyl-3-((6-(1-methyl-1H-pyrazol-4-yl)pyrazolo[1,5-a]pyrazin-4-yl)oxy)cyclobutyl)acrylamide

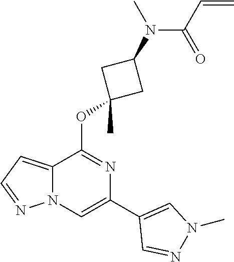 [SMILES: C=CC(=O)N(C)[C@H]1C[C@](C)(Oc2nc(-c3cnn(C)c3)cn3nccc23)C1]

### 1. Synthesis of (1r,3r)-N,3-dimethyl-3-((6-(1-methyl-1H-pyrazol-4-yl)pyrazolo[1,5-a]pyrazin-4-yl)oxy)cyclobutan-1-amine

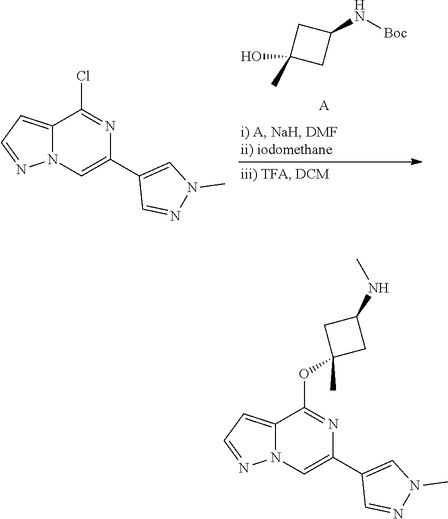

(1r,3r)-N,3-dimethyl-3-((6-(1-methyl-1H-pyrazol-4-yl)pyrazolo[1,5-a]pyrazin-4-yl)oxy)cyclobutan-1-amine was synthesized in the same way as Example 185 but starting from tert-butyl ((1r,3r)-3-hydroxy-3-methylcyclobutyl)carbamate (47 mg, 0.235 mmol). The crude material used in the next step without further purification (assuming 100% yield). LCMS m/z=313.1 (M+H)⁺.

### 2. Synthesis of N-methyl-N-((1r,3r)-3-methyl-3-((6-(1-methyl-1H-pyrazol-4-yl)pyrazolo[1,5-a]pyrazin-4-yl)oxy)cyclobutyl)acrylamide

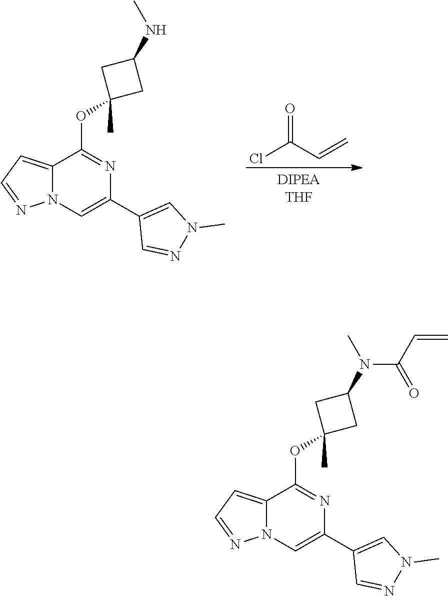

N-methyl-N-((1r,3r)-3-methyl-3-((6-(1-methyl-1H-pyrazol-4-yl)pyrazolo[1,5-a]pyrazin-4-yl)oxy)cyclobutyl)acrylamide was synthesized in the same way as Example 27 but starting from crude (1r,3r)-N,3-dimethyl-3-((6-(1-methyl-1H-pyrazol-4-yl)pyrazolo[1,5-a]pyrazin-4-yl)oxy)cyclobutan-1-amine (22 mg, 0.071 mmol). The material was purified by column chromatography (24 g SiO₂, 10-100% EtOH:EtOAc (2% NH₄OH)1:3 in heptane) yielding N-methyl-N—((1r,3r)-3-methyl-3-((6-(1-methyl-1H-pyrazol-4-yl)pyrazolo[1,5-a]pyrazin-4-yl)oxy)cyclobutyl)acrylamide (7 mg, 28% yield) as an off-white solid. LCMS m/z=389.1 (M+Na)⁺. ¹H NMR (500 MHz, acetonitrile-d₃) δ 8.34 ppm (s, 1H), 7.93 (s, 1H), 7.88 (d, J=2.44 Hz, 1H), 7.85 (s, 1H), 6.73-6.80 (m, 1H), 6.62 (br s, 1H), 5.92-6.29 (m, 1H), 5.34-5.75 (m, 1H), 4.61-5.12 (m, 1H), 3.89 (s, 3H), 2.98 (br s, 5H), 2.42-2.66 (m, 2H), 1.85 (s, 3H).

### Example 218: rac-N-((trans)-2,2-dimethyl-3-((6-(1-methyl-1H-pyrazol-4-yl)pyrazolo[1,5-a]pyrazin-4-yl)oxy)cyclobutyl)-N-methylacrylamide

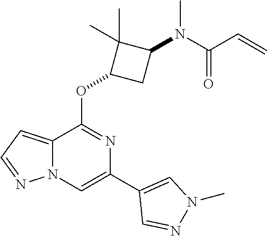

### 1. Synthesis of rac-(trans)-N,2,2-trimethyl-3-((6-(1-methyl-1H-pyrazol-4-yl)pyrazolo[1,5-a]pyrazin-4-yl)oxy)cyclobutan-1-amine

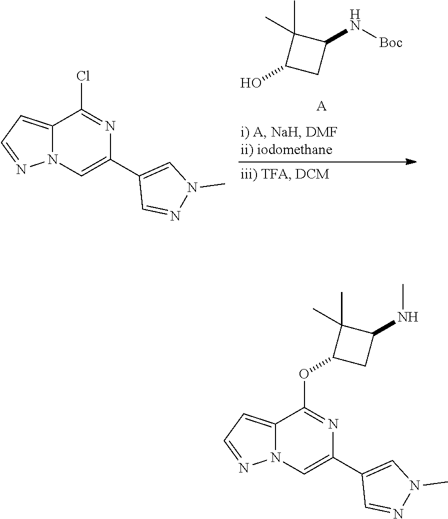

rac-(trans)-N,2,2-trimethyl-3-((6-(1-methyl-1H-pyrazol-4-yl)pyrazolo[1,5-a]pyrazin-4-yl)oxy)cyclobutan-1-amine was synthesized in the same way as Example 185 but starting from rac-tert-butyl ((trans)-3-hydroxy-2,2-dimethylcyclobutyl)carbamate (51 mg, 0.235 mmol). The crude material used in the next step without further purification (assuming 100% yield). LCMS m/z=327.1 (M+H)⁺.

### 2. Synthesis of rac-N-((trans)-2,2-dimethyl-3-((6-(1-methyl-1H-pyrazol-4-yl)pyrazolo[1,5-a]pyrazin-4-yl)oxy)cyclobutyl)-N-methylacrylamide

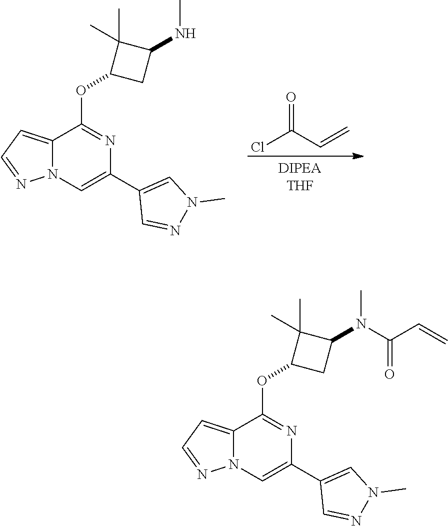

rac-N-((1s,3s)-2,2-Dimethyl-3-((6-(1-methyl-1H-pyrazol-4-yl)pyrazolo[1,5-a]pyrazin-4-yl)oxy)cyclobutyl)-N-methylacrylamide was synthesized in the same way as Example 27 but starting from crude rac-(1S,3S)—N,2,2-trimethyl-3-((6-(1-methyl-H-pyrazol-4-yl)pyrazolo[1,5-a]pyrazin-4-yl)oxy)cyclobutan-1-amine (30 mg, 0.092 mmol). The material was purified by column chromatography (24 g SiO2, 10-100% EtOH:EtOAc (2% NH₄OH)1:3 in heptane) to afford rac-N-((trans)-2,2-dimethyl-3-((6-(1-methyl-1H-pyrazol-4-yl)pyrazolo[1,5-a]pyrazin-4-yl)oxy)cyclobutyl)-N-methylacrylamide (29 mg, 82% yield) as an off-white solid. LCMS m/z=403.1 (M+Na)⁺. ¹H NMR (500 MHz, acetonitrile-d₃) δ ppm 8.26 (s, 1H), 7.84 (s, 1H), 7.81 (d, J=1.83 Hz, 1H), 7.78 (s, 1H), 6.71 (d, J=1.22 Hz, 1H), 6.45-6.69 (m, 1H), 6.08 (dd, J=2.14, 16.79 Hz, 1H), 5.57 (br d, J=10.38 Hz, 1H), 5.25 (br s, 1H), 4.19-4.58 (m, 1H), 3.80 (s, 3H), 3.06 (br s, 3H), 2.81 (br s, 1H), 2.51-2.68 (m, 1H), 1.05-1.14 (m, 6H).

### Examples 219 & 220: N-((1S,3S)-2,2-dimethyl-3-((6-(1-methyl-1H-pyrazol-4-yl)pyrazolo[1,5-a]pyrazin-4-yl)oxy)cyclobutyl)-N-methylacrylamide and N-((1R,3R)-2,2-dimethyl-3-((6-(1-methyl-1H-pyrazol-4-yl)pyrazolo[1,5-a]pyrazin-4-yl)oxy)cyclobutyl)-N-methylacrylamide

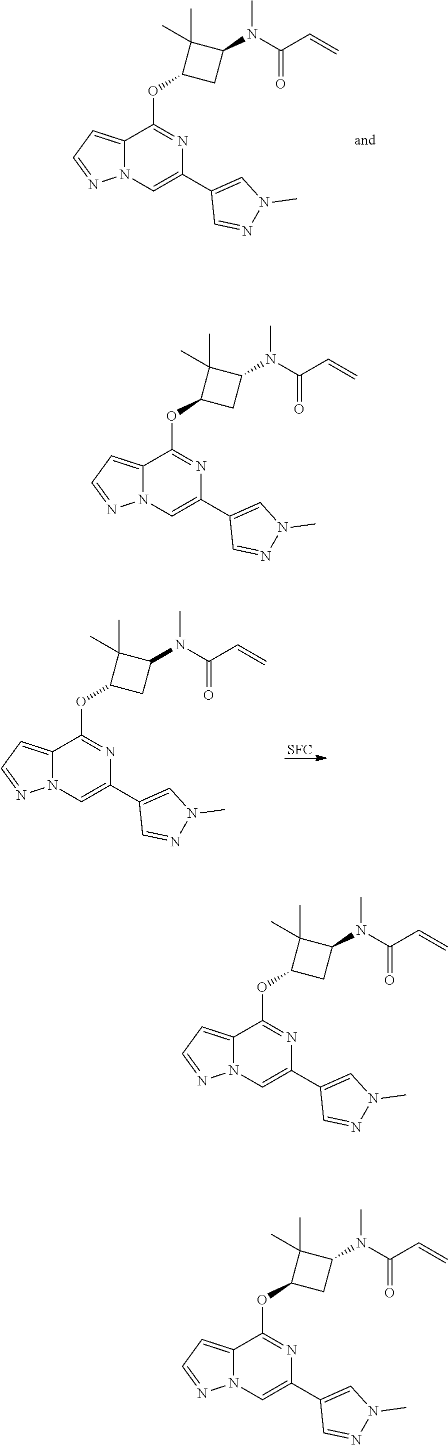

The title compounds were prepared from rac-N-((trans)-2,2-dimethyl-3-((6-(1-methyl-1H-pyrazol-4-yl)pyrazolo[1,5-a]pyrazin-4-yl)oxy)cyclobutyl)-N-methylacrylamide (Example 218) by prep-SFC (CHIRALPAK OX—H 30×250 mm, 5 um, Method: 40% MeOH w/no modifier in CO₂ (flow rate: 100 mL/min, ABPR 120 bar, MBPR 40 psi, column temp 40 deg C)) to afford:

*Peak 1, Example 21. LCMS m/z=403.1 (M+Na)⁺. ¹H NMR (500 MHz, MeCN-d₃) δ ppm 8.26 (s, 1H), 7.84 (s, 1H), 7.81 (d, J=1.83 Hz, 1H), 7.78 (s, 1H), 6.71 (d, J=1.22 Hz, 1H), 6.45-6.69 (m, 1H), 6.08 (dd, J=2.14, 16.79 Hz, 1H), 5.57 (br d, J=10.38 Hz, 1H), 5.25 (br s, 1H), 4.19-4.58 (m, 1H), 3.80 (s, 3H), 3.06 (br s, 3H), 2.81 (br s, 1H), 2.51-2.68 (m, 1H), 1.05-1.14 (m, 6H).

*Peak 2, Example 220. LCMS m/z=403.1 (M+Na)⁺. ¹H NMR (500 MHz, MeCN-d₃) δ ppm 8.26 (s, 1H), 7.84 (s, 1H), 7.81 (d, J=1.83 Hz, 1H), 7.78 (s, 1H), 6.71 (d, J=1.22 Hz, 1H), 6.45-6.69 (m, 1H), 6.08 (dd, J=2.14, 16.79 Hz, 1H), 5.57 (br d, J=10.38 Hz, 1H), 5.25 (br s, 1H), 4.19-4.58 (m, 1H), 3.80 (s, 3H), 3.06 (br s, 3H), 2.81 (br s, 1H), 2.51-2.68 (m, 1H), 1.05-1.14 (m, 6H).

### Example 221: 1-((1R,5S,6s)-6-(((6-(1-methyl-1H-pyrazol-4-yl)pyrazolo[1,5-a]pyrazin-4-yl)oxy)methyl)-3-azabicyclo[3.1.0]hexan-3-yl)prop-2-en-1-one

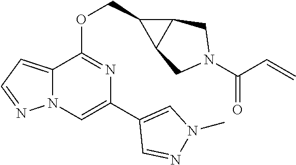 [SMILES: C=CC(=O)N1C[C@@H]2[C@H](COc3nc(-c4cnn(C)c4)cn4nccc34)[C@@H]2C1]

### 1. Synthesis of 4-(((1R,5S,6s)-3-azabicyclo[3.1.0]hexan-6-yl)methoxy)-6-(1-methyl-1H-pyrazol-4-yl)pyrazolo[1,5-a]pyrazine

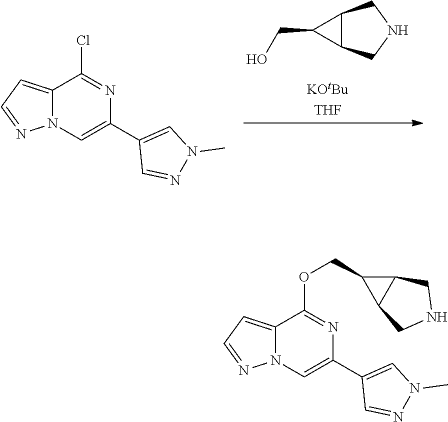 [SMILES: Cn1cc(-c2cn3nccc3c(Cl)n2)cn1 ; OC[C@H]1[C@H]2CNC[C@@H]12 ; Cn1cc(-c2cn3nccc3c(OC[C@@H]3[C@@H]4CNC[C@@H]43)n2)cn1]

4-(((1R,5S,6s)-3-azabicyclo[3.1.0]hexan-6-yl)methoxy)-6-(1-methyl-1H-pyrazol-4-yl)pyrazolo[1,5-a]pyrazine was synthesized in the same way as Example 214 but starting from ((1R,5S,6s)-3-azabicyclo[3.1.0]hexan-6-yl)methanol (27 mg, 0.235 mmol). The crude title compound was obtained as a pale orange residue which was used in the next step without further purification (assuming 100% yield). LCMS m/z=311.0 (M+H)⁺.

### 2. Synthesis of 1-((1R,5S,6s)-6-(((6-(1-methyl-1H-pyrazol-4-yl)pyrazolo[1,5-a]pyrazin-4-yl)oxy)methyl)-3-azabicyclo[3.1.0]hexan-3-yl)prop-2-en-1-one

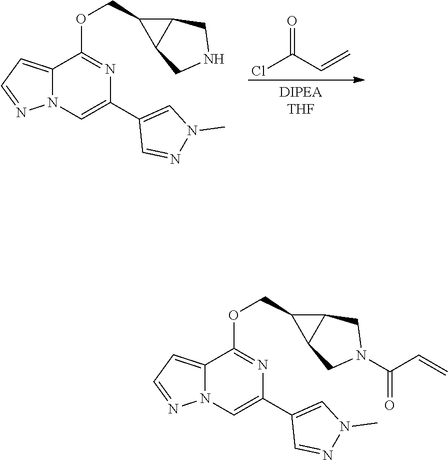

1-((1R,5S,6s)-6-(((6-(1-methyl-1H-pyrazol-4-yl)pyrazolo[1,5-a]pyrazin-4-yl)oxy)methyl)-3-azabicyclo[3.1.0]hexan-3-yl)prop-2-en-1-one was synthesized in the same way as Example 27 but starting from crude 4-(((1R,5S,6s)-3-azabicyclo[3.1.0]hexan-6-yl)methoxy)-6-(1-methyl-1H-pyrazol-4-yl)pyrazolo[1,5-a]pyrazine (66 mg, 0.213 mmol). The material was purified by column chromatography (24 g SiO2, 0-100% EtOH:EtOAc (2% NH₄OH)1:3 in heptane) yielding the title compound (37 mg, 48% yield) as an off-white solid. LCMS m/z=387.1 (M+Na)⁺. ¹H NMR (500 MHz, acetonitrile-d₃) δ ppm 8.24 (d, J=1.22 Hz, 1H), 7.85 (s, 1H), 7.80 (d, J=2.44 Hz, 1H), 7.75 (s, 1H), 6.63-6.70 (m, 1H), 6.32-6.40 (m, 1H), 6.02-6.08 (m, 1H), 5.48 (dd, J=2.44, 10.38 Hz, 1H), 4.39-4.59 (m, 2H), 3.79 (s, 3H), 3.50-3.73 (m, 4H), 1.75-1.90 (m, 3H).

### Example 222: N-((cis)-3-((7-fluoro-6-(1-methyl-1H-pyrazol-4-yl)pyrazolo[1,5-a]pyrazin-4-yl)oxy)cyclobutyl)-N-methylbut-2-ynamide

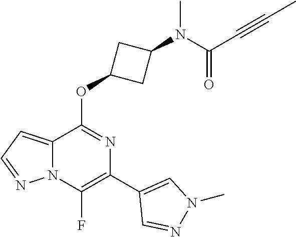 [SMILES: CC#CC(=O)N(C)[C@H]1C[C@@H](Oc2nc(-c3cnn(C)c3)c(F)n3nccc23)C1]

### 1. Synthesis of tert-butyl ((cis)-3-((7-fluoro-6-(1-methyl-1H-pyrazol-4-yl)pyrazolo[1,5-a]pyrazin-4-yl)oxy)cyclobutyl)(methyl)carbamate

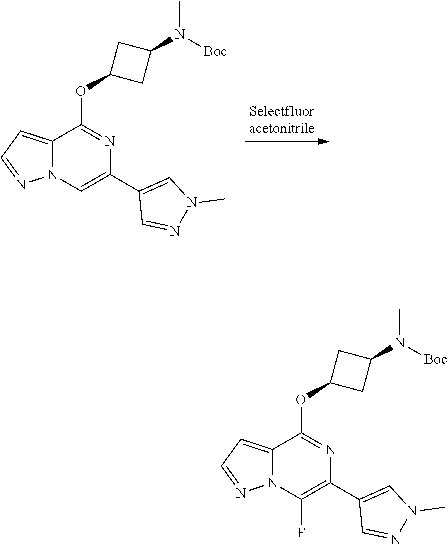 [SMILES: Cn1cc(-c2cn3nccc3c(O[C@H]3C[C@@H](N(C)C(=O)OC(C)(C)C)C3)n2)cn1 ; Cn1cc(-c2nc(O[C@H]3C[C@@H](N(C)C(=O)OC(C)(C)C)C3)c3ccnn3c2F)cn1]

To a solution of tert-butyl methyl((cis)-3-((6-(1-methyl-1H-pyrazol-4-yl)pyrazolo[1,5-a]pyrazin-4-yl)oxy)cyclobutyl)carbamate (84 mg, 0.211 mmol; step 1 Example 215) in acetonitrile (2.5 mL) was added Selectfluor (93 mg, 0.264 mmol) at RT. After 15 min at that temperature, EtOAc (2 mL) was added, the resulting precipitate was removed by filtration and the volatiles were removed. The crude residue was purified by column chromatography (24 g SiO2, 0-100% EtOH:EtOAc (2% NH₄OH)1:3 in heptane) yielding tert-butyl ((cis)-3-((7-fluoro-6-(1-methyl-1H-pyrazol-4-yl)pyrazolo[1,5-a]pyrazin-4-yl)oxy)cyclobutyl)(methyl)carbamate (88 mg, 70% yield) as an off-white solid. LCMS m/z=417.1 (M+H)⁺.

### 2. Synthesis of (cis)-3-((7-fluoro-6-(1-methyl-1H-pyrazol-4-yl)pyrazolo[1,5-a]pyrazin-4-yl)oxy)-N-methylcyclobutan-1-amine

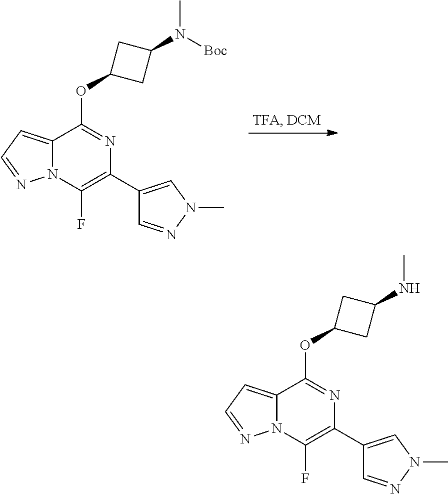 [SMILES: Cn1cc(-c2nc(O[C@H]3C[C@@H](N(C)C(=O)OC(C)(C)C)C3)c3ccnn3c2F)cn1 ; CN[C@H]1C[C@@H](Oc2nc(-c3cnn(C)c3)c(F)n3nccc23)C1]

(cis)-3-((7-fluoro-6-(1-methyl-1H-pyrazol-4-yl)pyrazolo[1,5-a]pyrazin-4-yl)oxy)-N-methylcyclobutan-1-amine was synthesized in the same way as Example 215 but starting from tert-butyl ((1s,3s)-3-((7-fluoro-6-(1-methyl-1H-pyrazol-4-yl)pyrazolo[1,5-a]pyrazin-4-yl)oxy)cyclobutyl)(methyl)carbamate (61 mg, 0.146 mmol). (cis)-3-((7-fluoro-6-(1-methyl-1H-pyrazol-4-yl)pyrazolo[1,5-a]pyrazin-4-yl)oxy)-N-methylcyclobutan-1-amine was obtained as a pale yellow residue (assuming 100% yield) which was used in the next without further purification. LCMS m/z=317.0 (M+H)⁺. 3. Synthesis of N-((cis)-3-((7-fluoro-6-(1-methyl-1H-pyrazol-4-yl)pyrazolo[1,5-a]pyrazin-4-yl)oxy)cyclobutyl)-N-methylbut-2-ynamide

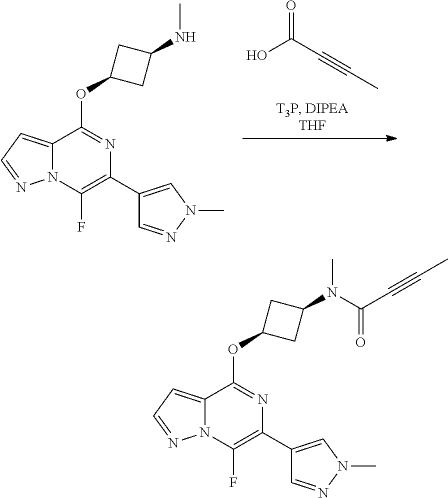

N-((cis)-3-((7-fluoro-6-(1-methyl-1H-pyrazol-4-yl)pyrazolo[1,5-a]pyrazin-4-yl)oxy)cyclobutyl)-N-methylbut-2-ynamide was synthesized in the same way as Example 1 (Step 3) but starting from (cis)-3-((7-fluoro-6-(1-methyl-1H-pyrazol-4-yl)pyrazolo[1,5-a]pyrazin-4-yl)oxy)-N-methylcyclobutan-1-amine (31 mg, 0.098 mmol) and using 2-butanoic acid (17 mg, 0.196 mmol). The crude material was purified by column chromatography (24 g SiO2, 0-100% EtOH:EtOAc (2% NH₄OH)1:3 in heptane) yielding N-((cis)-3-((7-fluoro-6-(1-methyl-1H-pyrazol-4-yl)pyrazolo[1,5-a]pyrazin-4-yl)oxy)cyclobutyl)-N-methylbut-2-ynamide (38 mg, 93% yield) as an off-white solid. LCMS m/z=383.1 (M+H)⁺. ¹H NMR (500 MHz, MeOH-d₄) δ ppm 8.13 (d, J=4.88 Hz, 1H), 8.10 (dd, J=2.14, 3.97 Hz, 1H), 7.98 (d, J=8.55 Hz, 1H), 6.92-6.99 (m, 1H), 5.23 (quind, J=7.17, 14.65 Hz, 1H), 4.91-4.97 (m, 1H), 4.61-4.70 (m, 1H), 3.99 (d, J=3.66 Hz, 3H), 2.92-3.06 (m, 4H), 2.42-2.64 (m, 2H), 2.05-2.14 (m, 3H).

### Example 223: N-(2-fluoroethyl)-N-((trans)-3-((6-(1-methyl-1H-pyrazol-4-yl)pyrazolo[1,5-a]pyrazin-4-yl)oxy)cyclobutyl)but-2-ynamide

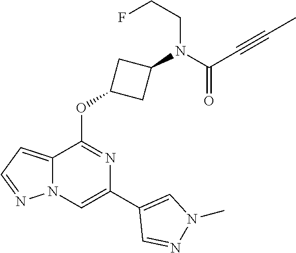 [SMILES: CC#CC(=O)N(CCF)[C@H]1C[C@H](Oc2nc(-c3cnn(C)c3)cn3nccc23)C1]

### 1. Synthesis of tert-butyl ((trans)-3-((6-(1-methyl-1H-pyrazol-4-yl)pyrazolo[1,5-a]pyrazin-4-yl)oxy)cyclobutyl)carbamate

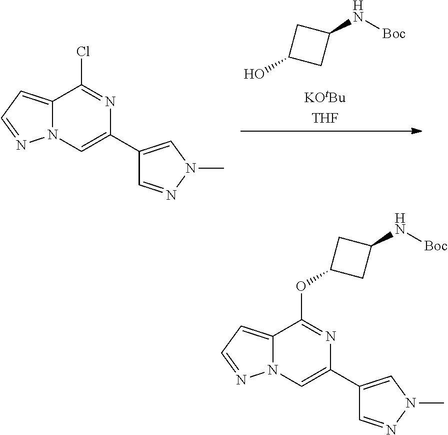 [SMILES: Cn1cc(-c2cn3nccc3c(Cl)n2)cn1 ; CC(C)(C)OC(=O)N[C@H]1C[C@H](O)C1 ; Cn1cc(-c2cn3nccc3c(O[C@H]3C[C@H](NC(=O)OC(C)(C)C)C3)n2)cn1]

tert-butyl ((trans)-3-((6-(1-methyl-1H-pyrazol-4-yl)pyrazolo[1,5-a]pyrazin-4-yl)oxy)cyclobutyl)carbamate was obtained as a pale orange residue (assuming 100% yield) which was used in the next without further purification in the same way as Example 214 but starting from tert-butyl ((trans)-3-hydroxycyclobutyl)carbamate (237 mg, 3.7 mmol). LCMS m/z=399.1 (M+H)⁺.

### 2. Synthesis of tert-butyl (2-fluoroethyl)((trans)-3-((6-(1-methyl-1H-pyrazol-4-yl)pyrazolo[1,5-a]pyrazin-4-yl)oxy)cyclobutyl)carbamate

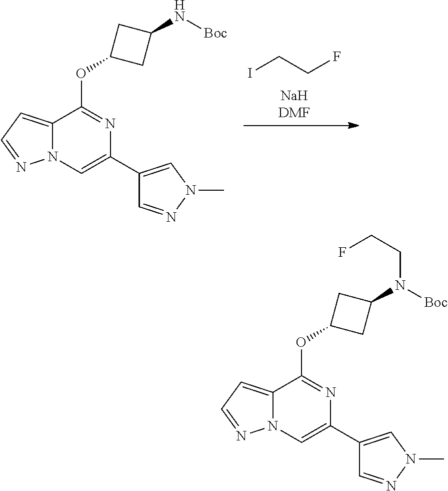

To a solution of tert-butyl (2-fluoroethyl)((trans)-3-((6-(1-methyl-1H-pyrazol-4-yl)pyrazolo[1,5-a]pyrazin-4-yl)oxy)cyclobutyl)carbamate (87 mg, 0.226 mmol). and 1-fluoro-2-iodoethane (49 mg, 0.283 mmol) in DMF (2 mL) was added sodium hydride (18 mg, 0.453 mmol) in one portion at RT. After stirring for overnight EtOAc (5 mL) was added to the vigorously stirred reaction mixture and the resulting organic phase was washed with sat. aq. NaHCO₃ (5 mL), water (5 mL), and brine (5 mL). The organic phase was separated, dried over Na₂SO₄, filtered, and concentrated under reduced pressure. The crude orange residue (assuming 100% yield) was used without further purification in the next step. LCMS m/z=431.2 (M+H)⁺.

### 3. Synthesis of (trans)-N-(2-fluoroethyl)-3-((6-(1-methyl-1H-pyrazol-4-yl)pyrazolo[1,5-a]pyrazin-4-yl)oxy)cyclobutan-1-amine

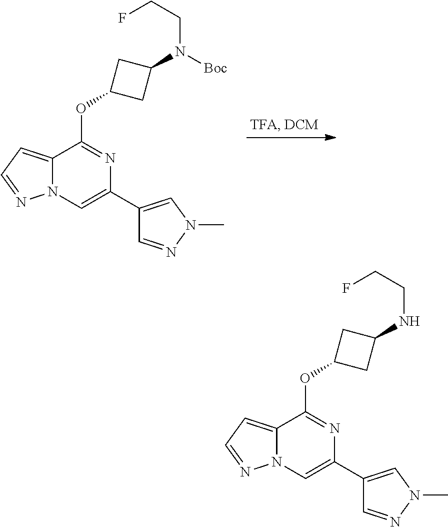 [SMILES: Cn1cc(-c2cn3nccc3c(O[C@H]3C[C@H](N(CCF)C(=O)OC(C)(C)C)C3)n2)cn1 ; Cn1cc(-c2cn3nccc3c(O[C@H]3C[C@H](NCCF)C3)n2)cn1]

(trans)-N-(2-fluoroethyl)-3-((6-(1-methyl-1H-pyrazol-4-yl)pyrazolo[1,5-a]pyrazin-4-yl)oxy)cyclobutan-1-amine was synthesized as a pale yellow solid (assume 100%) in the same way as Example 215 but starting from tert-butyl (2-fluoroethyl)((trans)-3-((6-(1-methyl-1H-pyrazol-4-yl)pyrazolo[1,5-a]pyrazin-4-yl)oxy)cyclobutyl)carbamate (97 mg, 0.226 mmol) and was used without further purification. LCMS m/z=331.1 (M+H)⁺.

### 4. Synthesis of N-(2-fluoroethyl)-N-((trans)-3-((6-(1-methyl-1H-pyrazol-4-yl)pyrazolo[1,5-a]pyrazin-4-yl)oxy)cyclobutyl)but-2-ynamide

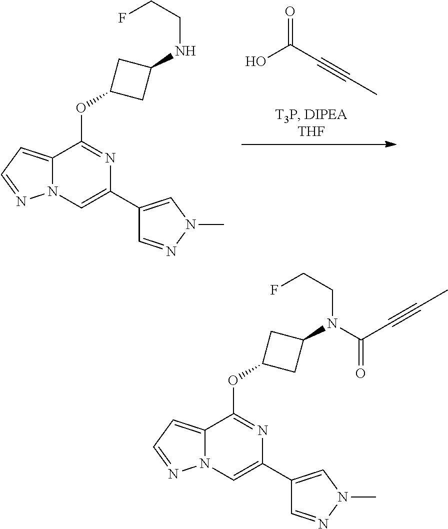

N-(2-fluoroethyl)-N-((trans)-3-((6-(1-methyl-1H-pyrazol-4-yl)pyrazolo[1,5-a]pyrazin-4-yl)oxy)cyclobutyl)but-2-ynamide was synthesized in the same way as Example 1 (Part 3) but starting from (trans)-N-(2-fluoroethyl)-3-((6-(1-methyl-1H-pyrazol-4-yl)pyrazolo[1,5-a]pyrazin-4-yl)oxy)cyclobutan-1-amine (37 mg, 0.113 mmol) and using 2-butanoic acid (14 mg, 0.170 mmol). The crude material was purified by prep HPLC (Column Waters XSelect CSH Prep C18 5 μm OBD 19×100 mm; Condition:5-60% Acetonitrile in 0.1% v/v Ammonium carbonate/water) to yield N-(2-fluoroethyl)-N-((1r,3r)-3-((6-(1-methyl-1H-pyrazol-4-yl)pyrazolo[1,5-a]pyrazin-4-yl)oxy)cyclobutyl)but-2-ynamide (5 mg, 12% yield) as an off-white solid. LCMS m/z=419.1 (M+Na)⁺. ¹H NMR (500 MHz, DMSO-d₆) δ ppm 8.68-8.82 (m, 1H), 8.16 (d, J=8.55 Hz, 1H), 7.93-8.09 (m, 2H), 6.82-7.01 (m, 1H), 5.42-5.62 (m, 2H), 5.14-5.30 (m, 1H), 4.46-4.74 (m, 2H), 3.90 (m, 3H), 3.71-3.81 (m, 1H), 2.78-2.96 (m, 2H), 2.57-2.69 (m, 2H), 2.04 (d, J=10.99 Hz, 3H).

### Example 224: N-(5-((6-(1-methyl-1H-pyrazol-4-yl)pyrazolo[1,5-a]pyrazin-4-yl)oxy)bicyclo[3.1.1]heptan-1-yl)acrylamide

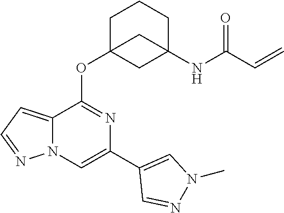 [SMILES: C=CC(=O)NC12CCCC(Oc3nc(-c4cnn(C)c4)cn4nccc34)(C1)C2]

### 1. Synthesis of 5-((6-(1-methyl-1H-pyrazol-4-yl)pyrazolo[1,5-a]pyrazin-4-yl)oxy)bicyclo[3.1.1]heptan-1-amine

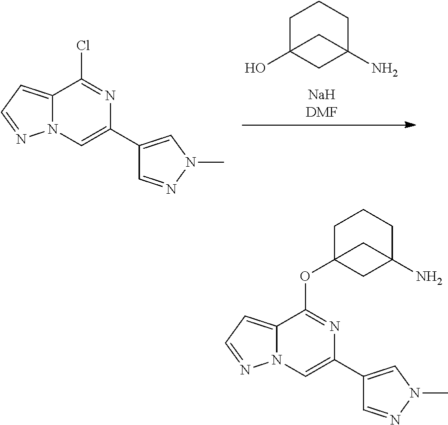 [SMILES: Cn1cc(-c2cn3nccc3c(Cl)n2)cn1 ; NC12CCCC(O)(C1)C2 ; Cn1cc(-c2cn3nccc3c(OC34CCCC(N)(C3)C4)n2)cn1]

To a solution of 4-chloro-6-(1-methyl-1H-pyrazol-4-yl)pyrazolo[1,5-a]pyrazine (50 mg, 214 μmol) and 5-aminobicyclo[3.1.1]heptan-1-ol (30 mg, 0.236 mmol) in DMF (2.5 mL) was added sodium hydride (26 mg, 0.642 mmol) in one portion at RT. The resulting reaction mixture was heated at 50 C for 30 min, brought back to RT and EtOH:EtOAc (1:3, 3 mL) was added. The reaction mixture was loaded onto a plug of silica and purified by column chromatography (12 g SiO₂, 0-100% EtOH:EtOAc (2% NH₄OH)1:3 in heptane) to afford 5-((6-(1-methyl-1H-pyrazol-4-yl)pyrazolo[1,5-a]pyrazin-4-yl)oxy)bicyclo[3.1.1]heptan-1-amine (36 mg, 52% yield) as an off-white solid. LCMS m/z=325.1 (M+H)⁺.

### 2. Synthesis of N-(5-((6-(1-methyl-1H-pyrazol-4-yl)pyrazolo[1,5-a]pyrazin-4-yl)oxy)bicyclo[3.1.1]heptan-1-yl)acrylamide

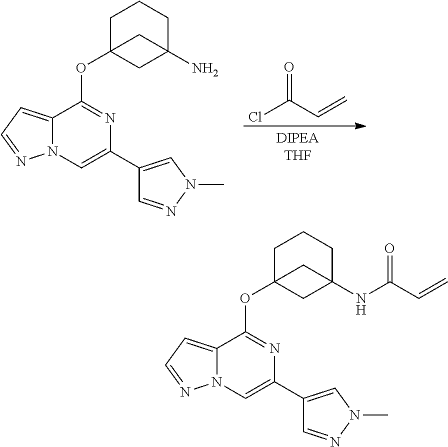

N-methyl-N-(5-((6-(1-methyl-1H-pyrazol-4-yl)pyrazolo[1,5-a]pyrazin-4-yl)oxy)bicyclo[3.1.1]heptan-1-yl)acrylamide was synthesized in the same way as Example 27 but starting from crude 5-((6-(1-methyl-1H-pyrazol-4-yl)pyrazolo[1,5-a]pyrazin-4-yl)oxy)bicyclo[3.1.1]heptan-1-amine (35 mg, 0.108 mmol). The material was purified by column chromatography (12 g SiO₂, 10-100% EtOH:EtOAc (2% NH₄OH)1:3 in heptane) yielding N-(5-((6-(1-methyl-1H-pyrazol-4-yl)pyrazolo[1,5-a]pyrazin-4-yl)oxy)bicyclo[3.1.1]heptan-1-yl)acrylamide (31 mg, 76% yield) as an off-white solid. LCMS m/z=401.1 (M+Na)⁺. ¹H NMR (500 MHz, MeOH-d₄) δ ppm 8.41 (d, J=1.22 Hz, 1H), 8.05 (s, 1H), 7.93 (s, 1H), 7.90 (d, J=2.44 Hz, 1H), 6.75 (dd, J=1.22, 2.44 Hz, 1H), 6.18-6.23 (m, 2H), 5.62 (dd, J=4.27, 7.32 Hz, 1H), 3.96 (s, 3H), 2.71-2.78 (m, 2H), 2.43-2.50 (m, 2H), 2.33 (t, J=6.41 Hz, 2H), 1.96-2.08 (m, 4H).

### Example 225: N-methyl-N-(5-((6-(1-methyl-1H-pyrazol-4-yl)pyrazolo[1,5-a]pyrazin-4-yl)oxy)bicyclo[3.1.1]heptan-1-yl)acrylamide

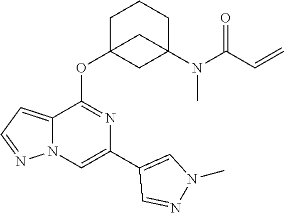 [SMILES: C=CC(=O)N(C)C12CCCC(Oc3nc(-c4cnn(C)c4)cn4nccc34)(C1)C2]

### Synthesis of N-methyl-N-(5-((6-(1-methyl-1H-pyrazol-4-yl)pyrazolo[1,5-a]pyrazin-4-yl)oxy)bicyclo[3.1.1]heptan-1-yl)acrylamide

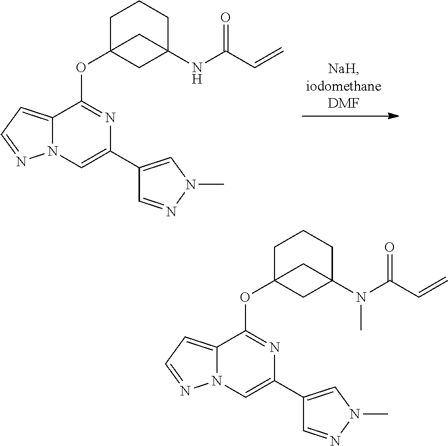 [SMILES: C=CC(=O)NC12CCCC(Oc3nc(-c4cnn(C)c4)cn4nccc34)(C1)C2 ; C=CC(=O)N(C)C12CCCC(Oc3nc(-c4cnn(C)c4)cn4nccc34)(C1)C2]

To a solution of N-(5-((6-(1-methyl-1H-pyrazol-4-yl)pyrazolo[1,5-a]pyrazin-4-yl)oxy)bicyclo[3.1.1]heptan-1-yl)acrylamide (24 mg, 0.063 mmol) and iodomethane (14 mg, 0.095 mmol) in DMF (2 mL) was added sodium hydride (8 mg, 0.190 mmol) at RT. After additional 30 min at RT it was diluted with MeOH (100 μL) and the reaction mixture was loaded onto a plug of silica and purified by column chromatography (12 g SiO₂, 10-100% EtOH:EtOAc (2% NH₄OH)1:3 in heptane) to give N-methyl-N-(5-((6-(1-methyl-1H-pyrazol-4-yl)pyrazolo[1,5-a]pyrazin-4-yl)oxy)bicyclo[3.1.1]heptan-1-yl)acrylamide (8.6 mg, 35% yield) as an off-white solid. LCMS m/z=415.1 (M+Na)⁺. ¹H NMR (500 MHz, MeOH-d₄) δ ppm 8.37-8.45 (m, 1H), 8.06 (s, 1H), 7.94 (s, 1H), 7.90 (d, J=2.44 Hz, 1H), 6.75 (d, J=1.83 Hz, 1H), 6.56-6.73 (m, 1H), 6.19 (dd, J=1.83, 16.48 Hz, 1H), 5.67-5.76 (m, 1H), 3.96 (s, 3H), 2.92-3.05 (m, 3H), 2.87 (br dd, J=2.14, 7.02 Hz, 2H), 2.27-2.61 (m, 4H), 1.95-2.14 (m, 4H).

### Example 226: N-(2,2-difluoroethyl)-N-((1s,3s)-3-methyl-3-((6-(1-methyl-1H-pyrazol-4-yl)pyrazolo[1,5-a]pyrazin-4-yl)oxy)cyclobutyl)acrylamide

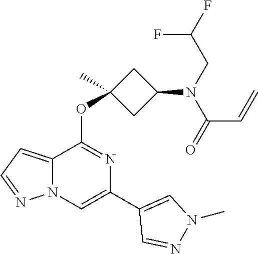 [SMILES: C=CC(=O)N(CC(F)F)[C@H]1C[C@@](C)(Oc2nc(-c3cnn(C)c3)cn3nccc23)C1]

### 1. Synthesis of tert-butyl ((1s,3s)-3-methyl-3-((6-(1-methyl-1H-pyrazol-4-yl)pyrazolo[1,5-a]pyrazin-4-yl)oxy)cyclobutyl)carbamate

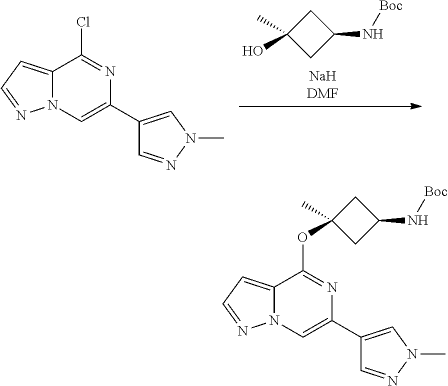 [SMILES: Cn1cc(-c2cn3nccc3c(Cl)n2)cn1 ; CC(C)(C)OC(=O)N[C@H]1C[C@@](C)(O)C1 ; Cn1cc(-c2cn3nccc3c(O[C@]3(C)C[C@H](NC(=O)OC(C)(C)C)C3)n2)cn1]

tert-butyl ((1s,3s)-3-methyl-3-((6-(1-methyl-1H-pyrazol-4-yl)pyrazolo[1,5-a]pyrazin-4-yl)oxy)cyclobutyl)carbamate was synthesized in the same way as Example 224 but starting from tert-butyl ((1s,3s)-3-hydroxy-3-methylcyclobutyl)carbamate (258 mg, 1.28 mmol). The reaction mixture was diluted with EtOAc (25 mL) washed with sat. aq. NH4Cl (10 mL), water (5 mL) and brine. The combined organics were dried (Na₂SO₄) and evaporated to dryness in vacuo to afford tert-butyl ((1s,3s)-3-methyl-3-((6-(1-methyl-1H-pyrazol-4-yl)pyrazolo[1,5-a]pyrazin-4-yl)oxy)cyclobutyl)carbamate as a pale orange residue (assuming 100% yield) which was used without further purification in the next step. LCMS m/z=399.2 (M+H)⁺.

### 2. Synthesis of (1s,3s)-3-methyl-3-((6-(1-methyl-1H-pyrazol-4-yl)pyrazolo[1,5-a]pyrazin-4-yl)oxy)cyclobutan-1-amine

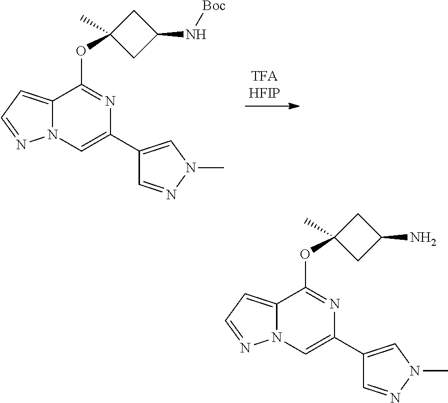 [SMILES: Cn1cc(-c2cn3nccc3c(O[C@]3(C)C[C@H](NC(=O)OC(C)(C)C)C3)n2)cn1 ; Cn1cc(-c2cn3nccc3c(O[C@]3(C)C[C@H](N)C3)n2)cn1]

To a solution of tert-butyl ((1s,3s)-3-methyl-3-((6-(1-methyl-1H-pyrazol-4-yl)pyrazolo[1,5-a]pyrazin-4-yl)oxy)cyclobutyl)carbamate (478 mg, 1.2 mmol) in HFIP (10 mL) was added TFA (275 μL, 2.4 mmol) at 0 C. The reaction mixture was allowed to warm to RT and stirred for overnight. EtOAc (50 mL) was added to the above mixture followed by the careful addition of sat. aq. NaHCO₃ (25 mL). The organic phase was separated and washed with water (10 mL) and brine (10 mL), dried (Na₂SO₄) and evaporated to dryness in vacuo. The residue was purified by column chromatography (24 g SiO₂, 80-100% EtOH:EtOAc (2% NH₄OH)1:3 in heptane) yielding (1s,3s)-3-methyl-3-((6-(1-methyl-1H-pyrazol-4-yl)pyrazolo[1,5-a]pyrazin-4-yl)oxy)cyclobutan-1-amine (263 mg, 74% yield) as an off-white solid. LCMS m/z=299.0 (M+H)⁺.

### 3. Synthesis of N-(2,2-difluoroethyl)-N—((1s,3s)-3-methyl-3-((6-(1-methyl-1H-pyrazol-4-yl)pyrazolo[1,5-a]pyrazin-4-yl)oxy)cyclobutyl)acrylamide

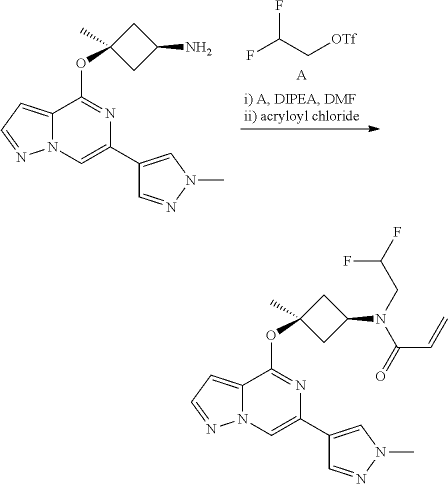

To a solution of (1s,3s)-3-methyl-3-((6-(1-methyl-1H-pyrazol-4-yl)pyrazolo[1,5-a]pyrazin-4-yl)oxy)cyclobutan-1-amine (50 mg, 0.168 mmol) and 2,2-difluoroethyl trifluoromethanesulfonate (40 μL, 0.190 mmol) in DMF (2 mL) was added DIPEA (90 μL, 0.505 mmol) at RT. After 1 h acryloyl chloride (30 μL, 0.340 mmol) was added and stirring was continued for an additional hour at RT. The reaction mixture was diluted with EtOAc (5 mL) and the organic phase was separated, washed with sat. aq. NH4Cl (5 mL), water (5 mL) and brine (5 mL). The combined organics were dried (Na₂SO₄) and concentrated. The residue was purified by column chromatography (24 g SiO₂, 10-100% EtOH:EtOAc (2% NH₄OH)_{1:3} in heptane) and prep HPLC (Column Waters XSelect CSH Prep C18 5 μm OBD 19×100 mm; Condition:5-75% Acetonitrile in 0.1% v/v Ammonium carbonate/water) to yield N-(2,2-difluoroethyl)-N-((1s,3s)-3-methyl-3-((6-(1-methyl-1H-pyrazol-4-yl)pyrazolo[1,5-a]pyrazin-4-yl)oxy)cyclobutyl)acrylamide (22 mg, 32% yield) as an off-white solid. LCMS m/z=439.1 (M+Na)⁺. ¹H NMR (500 MHz, MeOH-d₄) δ ppm 8.39 (s, 1H), 8.03 (s, 1H), 7.87-7.94 (m, 2H), 6.77 (s, 2H), 6.19-6.31 (m, 1H), 5.86-6.15 (m, 1H), 5.80 (br s, 1H), 4.20-4.54 (m, 1H), 3.69-4.04 (m, 5H), 2.89-2.98 (m, 2H), 2.71 (br s, 2H), 1.84 (s, 3H).

### Example 227: (E)-4-chloro-N-methyl-N-((trans)-3-((6-(1-methyl-1H-pyrazol-4-yl)pyrazolo[1,5-a]pyrazin-4-yl)oxy)cyclobutyl)but-2-enamide

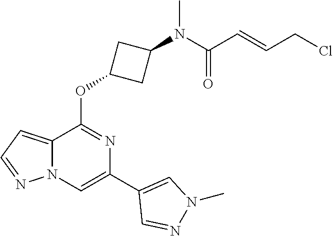 [SMILES: Cn1cc(-c2cn3nccc3c(O[C@H]3C[C@H](N(C)C(=O)/C=C/CCl)C3)n2)cn1]

### 1. Synthesis of (E)-4-chloro-N-methyl-N-((trans)-3-((6-(1-methyl-1H-pyrazol-4-yl)pyrazolo[1,5-a]pyrazin-4-yl)oxy)cyclobutyl)but-2-enamide

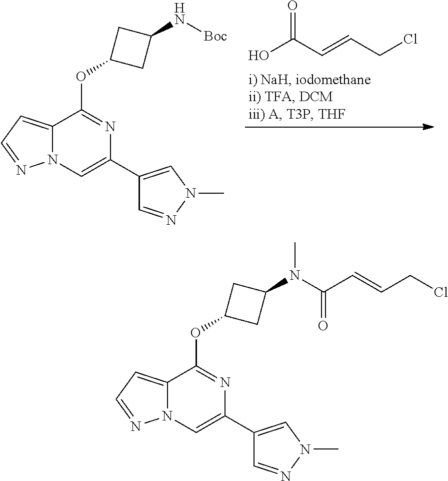 [SMILES: Cn1cc(-c2cn3nccc3c(O[C@H]3C[C@H](NC(=O)OC(C)(C)C)C3)n2)cn1 ; O=C(O)/C=C/CCl ; Cn1cc(-c2cn3nccc3c(O[C@H]3C[C@H](N(C)C(=O)/C=C/CCl)C3)n2)cn1]

To a solution of tert-butyl ((trans)-3-((6-(1-methyl-1H-pyrazol-4-yl)pyrazolo[1,5-a]pyrazin-4-yl)oxy)cyclobutyl)carbamate (101 mg, 0.264 mmol) and iodomethane (25 μL, 0.528 mmol) in DMF (5 mL) was added sodium hydride (21 mg, 0.528 mmol) at RT. After 30 min the reaction mixture was diluted with EtOAc (5 mL) and sat. aq. NaHCO₃ (5 mL) was added. The organic phase was separated and washed with (H₂O, 5 mL), brine (5 mL) and dried (Na₂SO₄) and concentrated evaporated to dryness. The residue was redissolved in DCM (2 mL) and TFA (750 μL) added at RT. After 1 h at RT the volatiles were removed under reduced pressure and resulting pale yellow residue and (E)-4-chlorobut-2-enoic acid (64 mg, 528 μmol) were dissolved in THF (2 mL). DIPEA (230 μL, 1.32 mmol) followed by T3P (336 mg, 528 μmol, 50% purity) was added to the mixture at RT and the reaction mixture stirred overnight at RT. The reaction mixture was diluted with EtOAc (5 mL) and sat. aq. NaHCO₃ (5 mL) was added. The organic phase was separated and washed water (5 mL) and brine (5 mL), dried (Na₂SO₄) and concentrated. The residue was purified by column chromatography (24 g SiO₂, 10-100% EtOH:EtOAc (2% NH₄OH)1:3 in heptane) and prep HPLC (Column Waters XSelect CSH Prep C18 5 μm OBD 19×100 mm; Condition:5-60% Acetonitrile in 0.1% v/v Ammonium carbonate/water) to yield (E)-4-chloro-N-methyl-N-((trans)-3-((6-(1-methyl-1H-pyrazol-4-yl)pyrazolo[1,5-a]pyrazin-4-yl)oxy)cyclobutyl)but-2-enamide (5.4 mg, 5% yield) as an off-white solid. LCMS m/z=423.1 (M+Na)⁺. ¹H NMR (500 MHz, DMSO-d₆) δ ppm 8.76 (s, 1H), 8.17 (s, 1H), 8.04 (d, J=2.44 Hz, 1H), 8.00 (s, 1H), 6.94-7.22 (m, 1H), 6.89 (br s, 1H), 6.71-6.79 (m, 1H), 6.63 (br s, 1H), 5.50 (br s, 1H), 4.83-5.32 (m, 1H), 4.38 (br s, 2H), 3.88 (s, 3H), 3.35 (s, 3H), 2.94-3.16 (m, 3H), 2.74-2.94 (m, 2H), 2.51-2.66 (m, 2H).

### Example 228: N-((1s,3s)-3-methyl-3-((6-(1-methyl-1H-pyrazol-4-yl)pyrazolo[1,5-a]pyrazin-4-yl)oxy)cyclobutyl)acrylamide

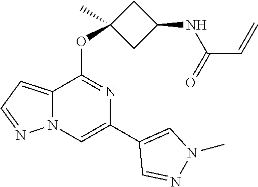 [SMILES: C=CC(=O)N[C@H]1C[C@@](C)(Oc2nc(-c3cnn(C)c3)cn3nccc23)C1]

### Synthesis of N—((1s,3s)-3-methyl-3-((6-(1-methyl-1H-pyrazol-4-yl)pyrazolo[1,5-a]pyrazin-4-yl)oxy)cyclobutyl)acrylamide

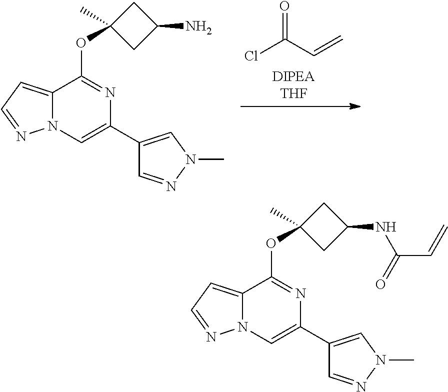

N-((1s,3s)-3-methyl-3-((6-(1-methyl-1H-pyrazol-4-yl)pyrazolo[1,5-a]pyrazin-4-yl)oxy)cyclobutyl)acrylamide was synthesized in the same way as Example 27 but starting from (1s,3s)-3-methyl-3-((6-(1-methyl-1H-pyrazol-4-yl)pyrazolo[1,5-a]pyrazin-4-yl)oxy)cyclobutan-1-amine (51 mg, 0.171 mmol) and using DIPEA (150 μL, 0.855 mmol) and acryloyl chloride (56 μL, 0.684 mmol). The material was purified by column chromatography (24 g SiO₂, 0-100% EtOH:EtOAc (2% NH₄OH)1:3 in heptane) yielding N-((1s,3s)-3-methyl-3-((6-(1-methyl-1H-pyrazol-4-yl)pyrazolo[1,5-a]pyrazin-4-yl)oxy)cyclobutyl)acrylamide (25 mg, 42% yield) as an off-white solid. LCMS m/z=375.1 (M+Na)⁺. ¹H NMR (500 MHz, MeOH-d₄) δ ppm 8.38 (d, J=1.22 Hz, 1H), 8.02 (s, 1H), 7.91 (s, 1H), 7.89 (d, J=1.83 Hz, 1H), 6.72-6.76 (m, 1H), 6.16-6.25 (m, 2H), 5.64 (dd, J=3.66, 8.55 Hz, 1H), 4.22-4.30 (m, 1H), 3.93 (s, 3H), 2.94 (ddd, J=3.05, 7.63, 10.07 Hz, 2H), 2.46-2.53 (m, 2H), 1.82 (s, 3H).

### Example 229: N-((1s,3s)-3-((3-cyclopropyl-6-(1-methyl-1H-pyrazol-4-yl)pyrazolo[1,5-a]pyrazin-4-yl)oxy)-3-methylcyclobutyl)-N-methylacrylamide

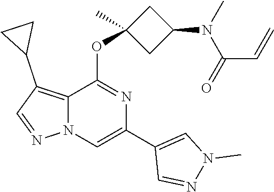 [SMILES: C=CC(=O)N(C)[C@H]1C[C@@](C)(Oc2nc(-c3cnn(C)c3)cn3ncc(C4CC4)c23)C1]

### 1. Synthesis of tert-butyl ((1s,3s)-3-((3-iodo-6-(1-methyl-1H-pyrazol-4-yl)pyrazolo[1,5-a]pyrazin-4-yl)oxy)-3-methylcyclobutyl)(methyl)carbamate

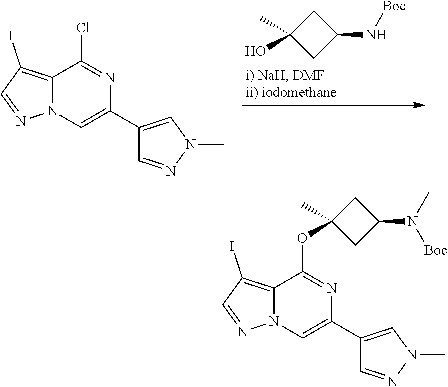 [SMILES: Cn1cc(-c2cn3ncc(I)c3c(Cl)n2)cn1 ; CC(C)(C)OC(=O)N[C@H]1C[C@@](C)(O)C1 ; Cn1cc(-c2cn3ncc(I)c3c(O[C@]3(C)C[C@H](N(C)C(=O)OC(C)(C)C)C3)n2)cn1]

To a solution of 4-chloro-3-iodo-6-(1-methyl-1H-pyrazol-4-yl)pyrazolo[1,5-a]pyrazine (893 mg, 2.48 mmol) and tert-butyl ((1s,3s)-3-hydroxy-3-methylcyclobutyl)carbamate (500 mg, 2.48 mmol) in DMF (12.5 mL) was added sodium hydride (298 mg, 7.45 mmol). After 3 h iodomethane (230 μL, 3.73 mmol) was added and stirring continued until all starting material was consumed by LCMS. To the reaction mixture was added EtOAc (25 mL) and the resulting organic phase was extracted with water (10 mL) and brine (10 mL). The combined organics were dried (Na₂SO₄) and concentrated. The residue was purified by column chromatography (24 g SiO2, 0-100% EtOH:EtOAc (2% NH₄OH)1:3 in heptane) to afford tert-butyl ((1s,3s)-3-((3-iodo-6-(1-methyl-1H-pyrazol-4-yl)pyrazolo[1,5-a]pyrazin-4-yl)oxy)-3-methylcyclobutyl)(methyl)carbamate (780 mg, 58% yield) as an off-white solid. LCMS m/z=539.0 (M+Na)⁺.

### 2. Synthesis of tert-butyl ((1s,3s)-3-((3-cyclopropyl-6-(1-methyl-1H-pyrazol-4-yl)pyrazolo[1,5-a]pyrazin-4-yl)oxy)-3-methylcyclobutyl)(methyl)carbamate

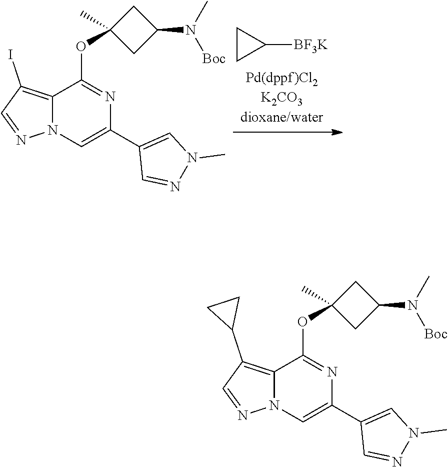

A reaction vial was charged with tert-butyl ((1s,3s)-3-((3-iodo-6-(1-methyl-1H-pyrazol-4-yl)pyrazolo[1,5-a]pyrazin-4-yl)oxy)-3-methylcyclobutyl)(methyl)carbamate (103 mg, 0.191 mmol), potassium carbonate (132 mg, 0.572 mmol), Pd(dppf)Cl2 (28 mg, 0.038 mmol) and potassium cyclopropyltrifluoroborate (56 mg, 0.381 mmol). The atmosphere in the reaction vial was exchanged by evaporating and backfilling with nitrogen three times. Dioxane (1.8 mL) and water (0.2 mL) were added to the vial and the resulting reaction mixture was purged with nitrogen for 15 min. The reaction mixture was heated at 80° C. for 8 h, brought back to RT and diluted with EtOAc (5 mL). The dark brown reaction mixture was loaded onto a plug of silica and subjected to column chromatography (24 g SiO₂, 0-100% EtOH:EtOAc (2% NH₄OH)1:3 in heptane) to afford tert-butyl ((1s,3s)-3-((3-cyclopropyl-6-(1-methyl-1H-pyrazol-4-yl)pyrazolo[1,5-a]pyrazin-4-yl)oxy)-3-methylcyclobutyl)(methyl)carbamate (20 mg, 23% yield) as an off-white solid. LCMS m/z=453.0 (M+H)⁺.

### 3. Synthesis of (1s,3s)-3-((3-cyclopropyl-6-(1-methyl-1H-pyrazol-4-yl)pyrazolo[1,5-a]pyrazin-4-yl)oxy)-N,3-dimethylcyclobutan-1-amine

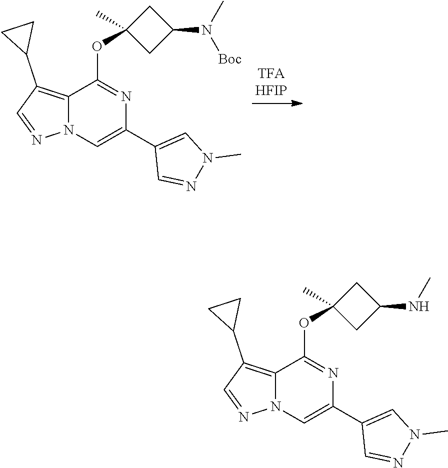 [SMILES: Cn1cc(-c2cn3ncc(C4CC4)c3c(O[C@]3(C)C[C@H](N(C)C(=O)OC(C)(C)C)C3)n2)cn1 ; CN[C@H]1C[C@@](C)(Oc2nc(-c3cnn(C)c3)cn3ncc(C4CC4)c23)C1]

The title compound was synthesized tert-butyl ((1s,3s)-3-((3-cyclopropyl-6-(1-methyl-1H-pyrazol-4-yl)pyrazolo[1,5-a]pyrazin-4-yl)oxy)-3-methylcyclobutyl)(methyl)carbamate (20 mg, 0.046 mmol) using an analogous method to Example 226. The crude material (assuming 100% yield) was used without purification in the next step. LCMS m/z=453.1 (M+H)⁺.

### 4. Synthesis of N-((1s,3s)-3-((3-cyclopropyl-6-(1-methyl-1H-pyrazol-4-yl)pyrazolo[1,5-a]pyrazin-4-yl)oxy)-3-methylcyclobutyl)-N-methylacrylamide

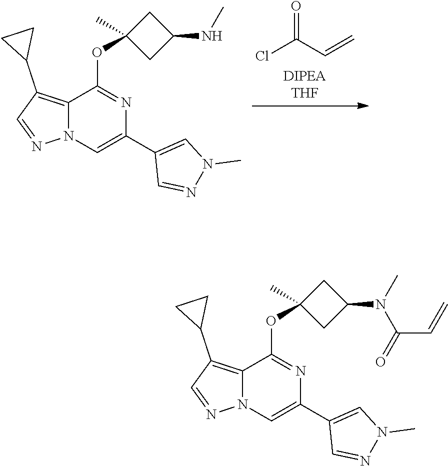

The title compound was synthesized in the same way as Example 27 but starting from crude (1s,3s)-3-((3-cyclopropyl-6-(1-methyl-1H-pyrazol-4-yl)pyrazolo[1,5-a]pyrazin-4-yl)oxy)-N,3-dimethylcyclobutan-1-amine (16 mg, 0.046 mmol). The material was purified by column chromatography (12 g SiO₂, 0-100% EtOH:EtOAc (2% NH₄OH)1:3 in heptane) yielding the title compound (2.8 mg, 15% yield) as an off-white solid. LCMS m/z=389.1 (M+Na)⁺. ¹H NMR (500 MHz, MeOH-d4) δ ppm 8.28 (s, 1H), 8.03 (s, 1H), 7.91 (s, 1H), 7.55 (s, 1H), 6.63-6.87 (m, 1H), 6.11-6.28 (m, 1H), 5.74 (br d, J=8.55 Hz, 1H), 4.50-4.79 (m, 1H), 3.94 (s, 3H), 2.97-3.11 (m, 3H), 2.65-2.91 (m, 4H), 2.23-2.33 (m, 1H), 1.89 (s, 3H), 0.97-1.07 (m, 2H), 0.64-0.77 (m, 2H).

### Example 230: N-methyl-N-((1s,3s)-3-methyl-3-((6-(1-methyl-1H-pyrazol-4-yl)pyrazolo[1,5-a]pyrazin-4-yl)oxy)cyclobutyl)cyclobut-1-ene-1-carboxamide

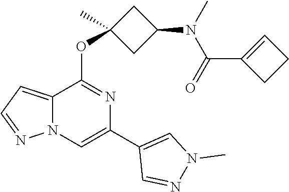 [SMILES: Cn1cc(-c2cn3nccc3c(O[C@]3(C)C[C@H](N(C)C(=O)C4=CCC4)C3)n2)cn1]

### Synthesis of N-methyl-N-((1s,3s)-3-methyl-3-((6-(1-methyl-1H-pyrazol-4-yl)pyrazolo[1,5-a]pyrazin-4-yl)oxy)cyclobutyl)cyclobut-1-ene-1-carboxamide

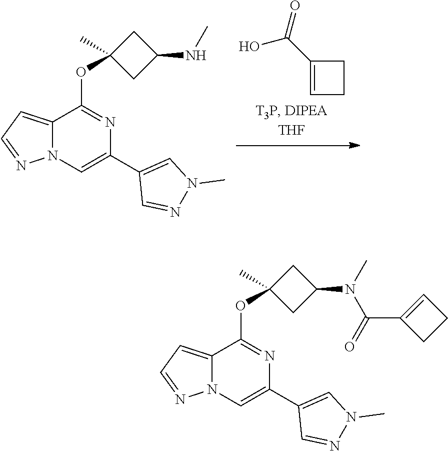 [SMILES: CN[C@H]1C[C@@](C)(Oc2nc(-c3cnn(C)c3)cn3nccc23)C1 ; O=C(O)C1=CCC1 ; Cn1cc(-c2cn3nccc3c(O[C@]3(C)C[C@H](N(C)C(=O)C4=CCC4)C3)n2)cn1]

The title compound was synthesized from (1s,3s)-N,3-dimethyl-3-((6-(1-methyl-1H-pyrazol-4-yl)pyrazolo[1,5-a]pyrazin-4-yl)oxy)cyclobutan-1-amine (25 mg, 0.080 mmol) and using cyclobut-1-ene-1-carboxylic acid (16 mg, 0.160 mmol) using an analogous method to that described for Example 1. The crude material was purified by prep HPLC (Column Waters XSelect CSH Prep C18 5 μm OBD 19×100 mm; Condition:5-75% Acetonitrile in 0.1% v/v Ammonium carbonate/water) to afford N-methyl-N-((1s,3s)-3-methyl-3-((6-(1-methyl-1H-pyrazol-4-yl)pyrazolo[1,5-a]pyrazin-4-yl)oxy)cyclobutyl)cyclobut-1-ene-1-carboxamide (5 mg, 12% yield) as an off-white solid. LCMS m/z=415.1 (M+Na)⁺. ¹H NMR (500 MHz, MeOH-d₄) δ ppm 8.40 (s, 1H), 8.04 (s, 1H), 7.87-7.97 (m, 2H), 6.77 (br s, 1H), 6.50-6.61 (m, 1H), 4.60-4.81 (m, 1H), 3.94 (s, 3H), 2.92-3.18 (m, 3H), 2.76-2.92 (m, 5H), 2.68 (br s, 1H), 2.41-2.61 (m, 2H), 1.84 (s, 3H).

### Example 231: (E)-4,4,4-trifluoro-N-((1s,3s)-3-methyl-3-((6-(1-methyl-1H-pyrazol-4-yl)pyrazolo[1,5-a]pyrazin-4-yl)oxy)cyclobutyl)but-2-enamide

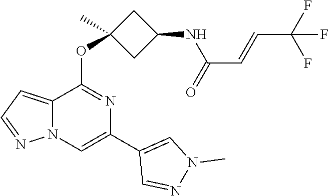 [SMILES: Cn1cc(-c2cn3nccc3c(O[C@]3(C)C[C@H](NC(=O)/C=C/C(F)(F)F)C3)n2)cn1]

### Synthesis of (E)-4,4,4-trifluoro-N—((1s,3s)-3-methyl-3-((6-(1-methyl-1H-pyrazol-4-yl)pyrazolo[1,5-a]pyrazin-4-yl)oxy)cyclobutyl)but-2-enamide

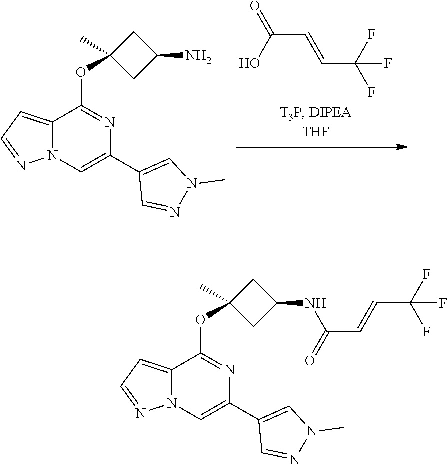 [SMILES: Cn1cc(-c2cn3nccc3c(O[C@]3(C)C[C@H](N)C3)n2)cn1 ; O=C(O)/C=C/C(F)(F)F ; Cn1cc(-c2cn3nccc3c(O[C@]3(C)C[C@H](NC(=O)/C=C/C(F)(F)F)C3)n2)cn1]

The title compound was synthesized in the same way as Example 1 but starting from (1s,3s)-3-methyl-3-((6-(1-methyl-1H-pyrazol-4-yl)pyrazolo[1,5-a]pyrazin-4-yl)oxy)cyclobutan-1-amine (30 mg, 0.100 mmol) and using (E)-4,4,4-trifluorobut-2-enoic acid (28 mg, 0.201 mmol). The crude material was purified by prep HPLC (Column Waters XSelect CSH Prep C18 5 μm OBD 19×100 mm; Condition:5-75% Acetonitrile in 0.1% v/v Ammonium carbonate/water) to afford (E)-4,4,4-trifluoro-N—((1s,3s)-3-methyl-3-((6-(1-methyl-1H-pyrazol-4-yl)pyrazolo[1,5-a]pyrazin-4-yl)oxy)cyclobutyl)but-2-enamide (25 mg, 60% yield) as an off-white solid. LCMS m/z=443.1 (M+Na)⁺. ¹H NMR (500 MHz, MeOH-d₄) δ ppm 8.39 (s, 1H), 8.03 (s, 1H), 7.87-7.94 (m, 2H), 6.68-6.79 (m, 2H), 6.60-6.68 (m, 1H), 4.27 (quin, J=8.09 Hz, 1H), 3.87-3.99 (m, 3H), 2.96 (ddd, J=2.75, 7.33, 10.07 Hz, 2H), 2.51 (dt, J=2.75, 9.31 Hz, 2H), 1.83 (s, 3H).

### Example 232: (E)-4,4,4-trifluoro-N-methyl-N-((1s,3s)-3-methyl-3-((6-(1-methyl-1H-pyrazol-4-yl)pyrazolo[1,5-a]pyrazin-4-yl)oxy)cyclobutyl)but-2-enamide

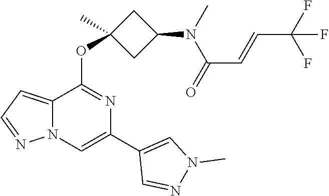 [SMILES: Cn1cc(-c2cn3nccc3c(O[C@]3(C)C[C@H](N(C)C(=O)/C=C/C(F)(F)F)C3)n2)cn1]

### Synthesis of (E)-4,4,4-trifluoro-N-methyl-N-((1s,3s)-3-methyl-3-((6-(1-methyl-1H-pyrazol-4-yl)pyrazolo[1,5-a]pyrazin-4-yl)oxy)cyclobutyl)but-2-enamide

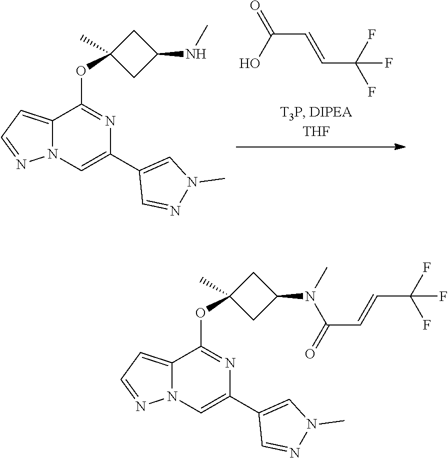 [SMILES: CN[C@H]1C[C@@](C)(Oc2nc(-c3cnn(C)c3)cn3nccc23)C1 ; O=C(O)/C=C/C(F)(F)F ; Cn1cc(-c2cn3nccc3c(O[C@]3(C)C[C@H](N(C)C(=O)/C=C/C(F)(F)F)C3)n2)cn1]

The title compound was synthesized in the same way as Example 1 but starting from (1s,3s)-N,3-dimethyl-3-((6-(1-methyl-1H-pyrazol-4-yl)pyrazolo[1,5-a]pyrazin-4-yl)oxy)cyclobutan-1-amine (25 mg, 0.080 mmol) and using (E)-4,4,4-trifluorobut-2-enoic acid (22 mg, 0160 mmol). The crude material was purified by prep HPLC (Column Waters XSelect CSH Prep C18 5 μm OBD 19×100 mm; Condition:5-75% Acetonitrile in 0.1% v/v Ammonium carbonate/water) to afford (E)-4,4,4-trifluoro-N-methyl-N-((1s,3s)-3-methyl-3-((6-(1-methyl-1H-pyrazol-4-yl)pyrazolo[1,5-a]pyrazin-4-yl)oxy)cyclobutyl)but-2-enamide (25 mg, 73% yield) as an off-white solid. LCMS m/z=457.1 (M+Na)⁺. ¹H NMR (500 MHz, MeOH-d₄) δ ppm 8.40 (d, J=4.88 Hz, 1H), 8.03 (d, J=3.05 Hz, 1H), 7.88-7.93 (m, 2H), 7.29 (dd, J=2.14, 15.57 Hz, 1H), 7.18 (dd, J=1.83, 15.26 Hz, 1H), 6.77 (dd, J=1.83, 6.71 Hz, 1H), 6.63-6.74 (m, 1H), 4.60-4.74 (m, 1H), 4.45 (quin, J=8.24 Hz, 1H), 3.08 (s, 2H), 3.00 (s, 1H), 2.78-2.92 (m, 3H), 2.66-2.76 (m, 1H), 1.85 (d, J=2.44 Hz, 3H).

### Example 233: N-(2,2-difluoroethyl)-N-((1s,3s)-3-methyl-3-((6-(1-methyl-1H-pyrazol-4-yl)pyrazolo[1,5-a]pyrazin-4-yl)oxy)cyclobutyl)but-2-ynamide

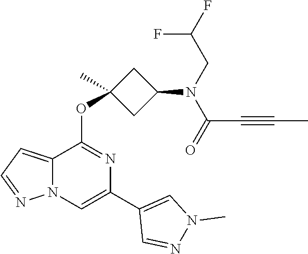 [SMILES: CC#CC(=O)N(CC(F)F)[C@H]1C[C@@](C)(Oc2nc(-c3cnn(C)c3)cn3nccc23)C1]

### Synthesis of N-(2,2-difluoroethyl)-N-((1s,3s)-3-methyl-3-((6-(1-methyl-1H-pyrazol-4-yl)pyrazolo[1,5-a]pyrazin-4-yl)oxy)cyclobutyl)but-2-ynamide

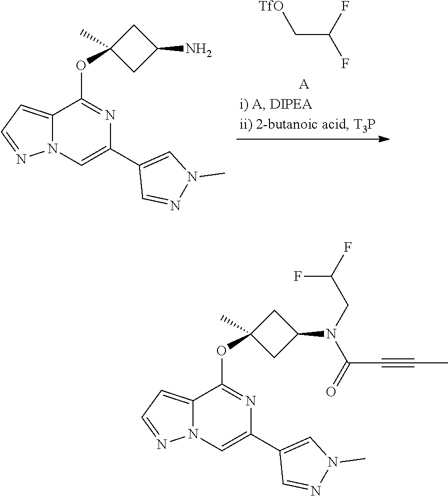

To a solution of (1s,3s)-3-methyl-3-((6-(1-methyl-1H-pyrazol-4-yl)pyrazolo[1,5-a]pyrazin-4-yl)oxy)cyclobutan-1-amine (106 μmol, 0.355 mmol) and 2,2-difluoroethyl trifluoromethanesulfonate (105 mg, 0.533 mmol) in DMF (2 mL) was added DIPEA (310 μL, 1.78 mmol) at RT and stirred for 1 h. To this were added 2-butynoic acid (60 mg, 0.711 mmol) and T3P (452 mg, 0.710 mmol, 50% purity) sequentially to the above reaction mixture at RT. The reaction mixture was stirred at RT for 1 h. The reaction mixture was diluted with EtOAc (10 mL) and sat. aq. NH4Cl (10 mL) and the organic phase washed with water (10 mL) and brine (10 mL). The combined organics were dried (Na₂SO₄) and evaporated to dryness in vacuo. The residue was purified by column chromatography (24 g SiO₂, 0-100% EtOH:EtOAc (2% NH₄OH)1:3 in heptane) to afford of N-(2,2-difluoroethyl)-N—((1s,3s)-3-methyl-3-((6-(1-methyl-1H-pyrazol-4-yl)pyrazolo[1,5-a]pyrazin-4-yl)oxy)cyclobutyl)but-2-ynamide (23 mg, 15% yield) as an off-white solid. LCMS m/z=451.2 (M+Na)⁺. ¹H NMR (500 MHz, MeOH-d₄) δ ppm 8.37 (dd, J=1.22, 10.38 Hz, 1H), 8.01 (d, J=9.16 Hz, 1H), 7.86-7.93 (m, 2H), 6.67-6.82 (m, 1H), 5.83-6.18 (m, 1H), 4.16-4.81 (m, 1H), 3.97-4.12 (m, 1H), 3.93 (d, J=1.83 Hz, 3H), 3.78 (dt, J=4.27, 14.04 Hz, 1H), 2.82-2.97 (m, 2H), 2.61-2.82 (m, 2H), 1.98-2.16 (m, 3H), 1.82 (d, J=19.53 Hz, 3H).

### Examples 234 & 235: 1-((1R,2R,4S)-2-((6-(1-methyl-1H-pyrazol-4-yl)pyrazolo[1,5-a]pyrazin-4-yl)oxy)-7-azabicyclo[2.2.1]heptan-7-yl)prop-2-en-1-one and 1-((1S,2S,4R)-2-((6-(1-methyl-1H-pyrazol-4-yl)pyrazolo[1,5-a]pyrazin-4-yl)oxy)-7-azabicyclo[2.2.1]heptan-7-yl)prop-2-en-1-one

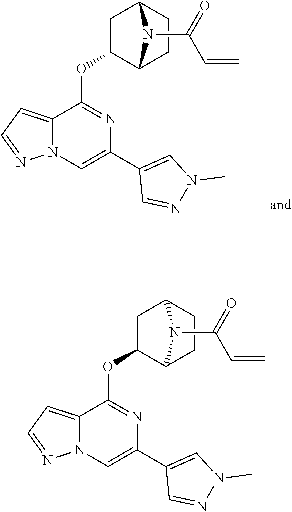

### 1. Synthesis of rac-tert-butyl (1R,2R,4S)-2-((6-(1-methyl-1H-pyrazol-4-yl)pyrazolo[1,5-a]pyrazin-4-yl)oxy)-7-azabicyclo[2.2.1]heptane-7-carboxylate

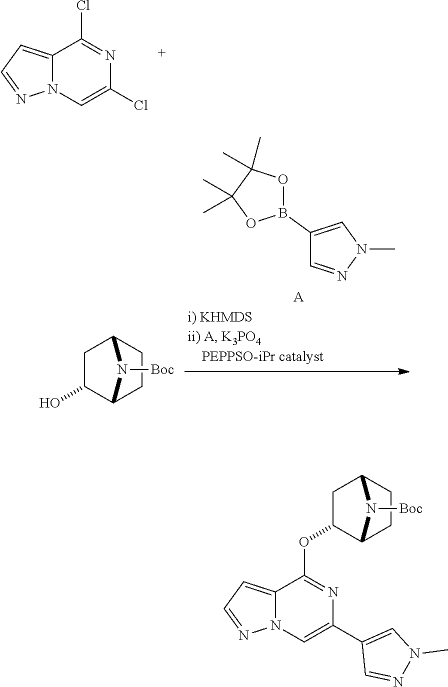

To solution of rac-tert-butyl (1R,2R,4S)-2-hydroxy-7-azabicyclo[2.2.1]heptane-7-carboxylate (1 g, 4.69 mmol) in dioxane (40 mL) was added KHMDS (8.53 mL, 1M in THF) at RT. The reaction mixture was stirred for 15 mins before a solution of 4,6-dichloropyrazolo[1,5-a]pyrazine (801 mg, 4.26 mmol) in dioxane (20 mL) was added dropwise and the resulting mixture stirred for 30 mins. The reaction mixture was degassed by purging with nitrogen for 15 min. To this was added PEPPSI-iPr catalyst (290 mg, 0.426 mmol) followed by previously degassed solutions of K₃PO₄ (1.81 g, 8.53 mmol) in water (10 mL) and 1-methyl-4-(4,4,5,5-tetramethyl-1,3,2-dioxaborolan-2-yl)-1H-pyrazole (1.77 g, 8.53 mmol) in dioxane (10 mL). Nitrogen was bubbled through the resulting mixture for an additional 15 min before the reaction mixture was heated at reflux for 2 h. The reaction was partitioned between and brine (50 mL) and EtOAc (50 mL) and the resulting biphasic mixture passed through a plug of Celite. The organic phase was separated, dried (Na₂SO₄) and evaporated to dryness in vacuo. The residue was purified by column chromatography (40 g SiO₂, 0-100% EtOH:EtOAc (2% NH₄OH)1:3 in heptane) afforded rac-tert-butyl (1R,2R,4S)-2-((6-(1-methyl-1H-pyrazol-4-yl)pyrazolo[1,5-a]pyrazin-4-yl)oxy)-7-azabicyclo[2.2.1]heptane-7-carboxylate as a dark gum (assuming 100% yield) which was used in the next step without further purification. LCMS m/z=411.1 (M+H)⁺.

### 2. Synthesis of rac-4-(((1R,2R,4S)-7-azabicyclo[2.2.1]heptan-2-yl)oxy)-6-(1-methyl-1H-pyrazol-4-yl)pyrazolo[1,5-a]pyrazine

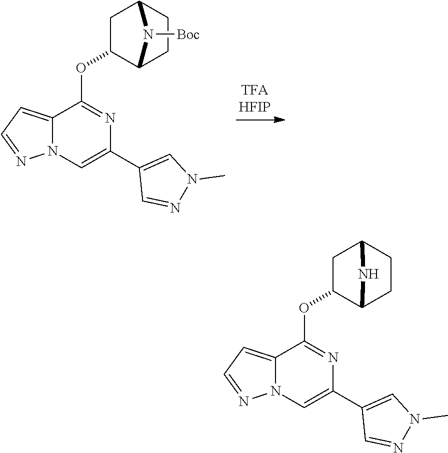

The title compound was synthesized from (1R,2R,4S)-2-((6-(1-methyl-1H-pyrazol-4-yl)pyrazolo[1,5-a]pyrazin-4-yl)oxy)-7-azabicyclo[2.2.1]heptane-7-carboxylate (1.75 g, 4.26 mmol) using an analogous method to that described for Example 226. Column chromatography (24 g SiO₂, 60-100% EtOH:EtOAc (2% NH₄OH)1:3 in heptane) afforded rac-4-(((1R,2R,4S)-7-azabicyclo[2.2.1]heptan-2-yl)oxy)-6-(1-methyl-1H-pyrazol-4-yl)pyrazolo[1,5-a]pyrazine as an orange oil (676 mg, 51% yield). LCMS m/z=311.1 (M+H)t.

### 3. Synthesis of rac-1-((1R,2R,4S)-2-((6-(1-methyl-1H-pyrazol-4-yl)pyrazolo[1,5-a]pyrazin-4-yl)oxy)-7-azabicyclo[2.2.1]heptan-7-yl)prop-2-en-1-one

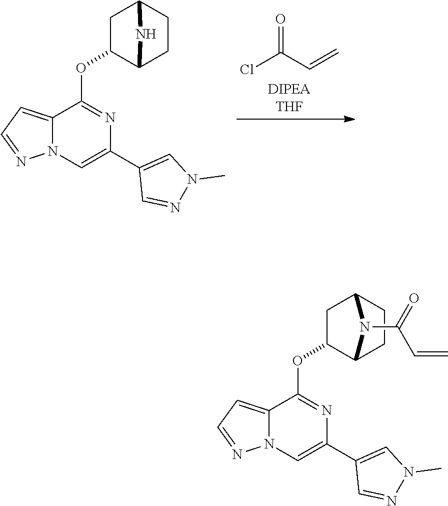

The title compound was synthesized in the same way as Example 27 but starting from 4-(((1R,2R,4S)-7-azabicyclo[2.2.1]heptan-2-yl)oxy)-6-(1-methyl-1H-pyrazol-4-yl)pyrazolo[1,5-a]pyrazine (676 mg, 2.18 mmol). The material was purified by column chromatography (40 g SiO2, 0-100% EtOH:EtOAc (2% NH₄OH)1:3 in heptane) to afford rac-1-((1R,2R,4S)-2-((6-(1-methyl-1H-pyrazol-4-yl)pyrazolo[1,5-a]pyrazin-4-yl)oxy)-7-azabicyclo[2.2.1]heptan-7-yl)prop-2-en-1-one (786 mg, 99% yield) as an off-white solid. LCMS m/z=365.1 (M+H)⁺.

### Preparation of 1-((1R,2R,4S)-2-((6-(1-methyl-1H-pyrazol-4-yl)pyrazolo[1,5-a]pyrazin-4-yl)oxy)-7-azabicyclo[2.2.1]heptan-7-yl)prop-2-en-1-one and 1-((1S,2S,4R)-2-((6-(1-methyl-1H-pyrazol-4-yl)pyrazolo[1,5-a]pyrazin-4-yl)oxy)-7-azabicyclo[2.2.1]heptan-7-yl)prop-2-en-1-one

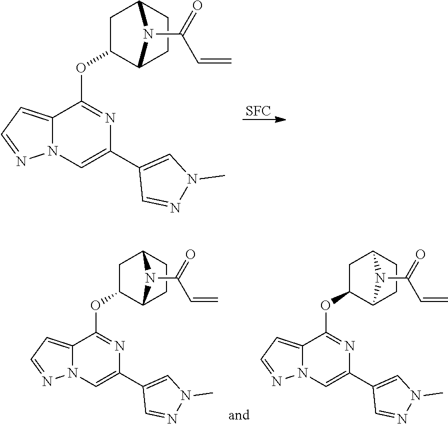

rac-1-((1R,2R,4S)-2-((6-(1-methyl-1H-pyrazol-4-yl)pyrazolo[1,5-a]pyrazin-4-yl)oxy)-7-azabicyclo[2.2.1]heptan-7-yl)prop-2-en-1-one (786 mg, 2.16 mmol) was separated by prep-SFC (CHIRALPAK IG 30×250 mm, 5 um, Method: 50% MeOH w/no modifier in CO₂ (flow rate: 100 mL/min, ABPR 120 bar, MBPR 40 psi, column temp 40 deg C)) to afford:

*Peak 1 (or E1), Example 234; (246 mg); LCMS m/z=365.1 (M+H)⁺. ¹H NMR (400 MHz, MeOH-d₄) δ 8.25 (s, 1H), 7.85-7.99 (m, 1H), 7.73-7.85 (m, 2H), 6.68 (d, J=2.01 Hz, 1H), 6.50-6.64 (m, 1H), 6.24 (br d, J=16.82 Hz, 1H), 5.66-5.77 (m, 1H), 5.18-5.34 (m, 1H), 4.80-5.09 (m, 1H), 4.45-4.63 (m, 1H), 3.81 (s, 3H), 2.34-2.52 (m, 1H), 1.95-2.20 (m, 1H), 1.37-1.87 (m, 4H).

*Peak 2 (or E2), Example 235; (252 mg); LCMS m/z=365.1 (M+H)⁺. ¹H NMR (400 MHz, MeOH-d₄) δ 8.25 (s, 1H), 7.85-7.99 (m, 1H), 7.73-7.85 (m, 2H), 6.68 (d, J=2.01 Hz, 1H), 6.50-6.64 (m, 1H), 6.24 (br d, J=16.82 Hz, 1H), 5.66-5.77 (m, 1H), 5.18-5.34 (m, 1H), 4.80-5.09 (m, 1H), 4.45-4.63 (m, 1H), 3.81 (s, 3H), 2.34-2.52 (m, 1H), 1.95-2.20 (m, 1H), 1.37-1.87 (m, 4H).

### Example 236: N-methyl-N-((1s,3s)-3-methyl-3-((7-(1-methyl-1H-pyrazol-4-yl)imidazo[1,2-c]pyrimidin-5-yl)oxy)cyclobutyl)acrylamide

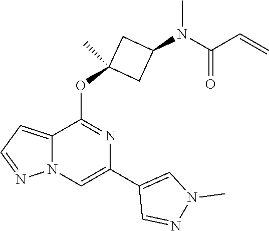 [SMILES: C=CC(=O)N(C)[C@H]1C[C@@](C)(Oc2nc(-c3cnn(C)c3)cn3nccc23)C1]

### 1. Synthesis of tert-butyl methyl((1s,3s)-3-methyl-3-((7-(1-methyl-1H-pyrazol-4-yl)imidazo[1,2-c]pyrimidin-5-yl)oxy)cyclobutyl)carbamate

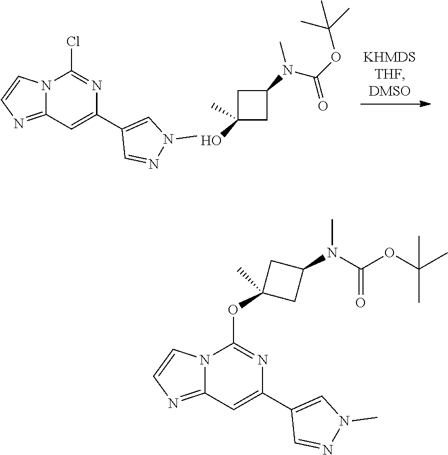

To a solution of tert-butyl ((1s,3s)-3-hydroxy-3-methylcyclobutyl)(methyl)carbamate (800 mg, 3.72 mmol) in dry THF (8 mL) under a N₂ atmosphere in an ice-water cooling bath was added a KHMDS solution (1M in THF, 8.4 mL). The reaction was stirred in the ice-water bath for 15 mins before a solution of 5-chloro-7-(1-methyl-1H-pyrazol-4-yl)imidazo[1,2-c]pyrimidine (650 mg, 2.78 mmol) in DMSO (8 mL) was added. The combined reaction mixture was allowed to warm to ambient temperature and was stirred for 30 mins. Water was added (20 mL), followed by 1 N HCl solution until pH=7, and EtOAc (50 mL). The layers were separated, and the aqueous phase was extracted with additional EtOAc (50 mL×3). The combined organic phase was washed sequentially with water and brine, then dried (Na₂SO₄), filtered, and concentrated. The crude material was purified by silica gel column chromatography (grading from 0-100% [3:1 EtOAc/EtOH] in heptanes) to give tert-butyl methyl((1s,3s)-3-methyl-3-((7-(1-methyl-1H-pyrazol-4-yl)imidazo[1,2-c]pyrimidin-5-yl)oxy)cyclobutyl)carbamate as an off-white solid (282 mg, 25% yield). ESI-MS (M+H)⁺: 413.2. ¹H NMR (500 MHz, MEOH-d₄) δ: 8.15 (s, 1H), 8.00 (s, 1H), 7.68 (d, J=1.8 Hz, 1H), 7.48 (d, J=1.2 Hz, 1H), 7.30 (s, 1H), 4.45-4.21 (br s, 1H), 3.95 (s, 3H), 2.84 (s, 3H), 2.83-2.73 (m, 4H), 1.87 (s, 3H), 1.47 (s, 9H).

### 2. Synthesis of (1s,3s)-N,3-dimethyl-3-((7-(1-methyl-1H-pyrazol-4-yl)imidazo[1,2-c]pyrimidin-5-yl)oxy)cyclobutan-1-amine

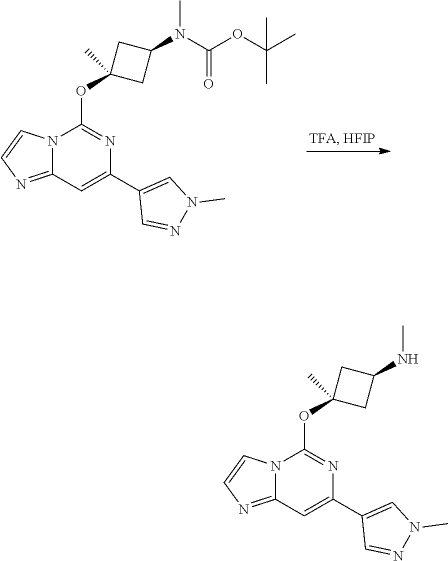 [SMILES: Cn1cc(-c2cc3nccn3c(O[C@]3(C)C[C@H](N(C)C(=O)OC(C)(C)C)C3)n2)cn1 ; CN[C@H]1C[C@@](C)(Oc2nc(-c3cnn(C)c3)cc3nccn23)C1]

A solution of tert-butyl methyl((1s,3s)-3-methyl-3-((7-(1-methyl-1H-pyrazol-4-yl)imidazo[1,2-c]pyrimidin-5-yl)oxy)cyclobutyl)carbamate (282 mg, 684 μmol) in hexafluoroisopropanol (4.7 mL) was cooled in an ice-water bath before TFA (168 μL, 2.20 mmol,) was added dropwise. The reaction mixture was warmed to ambient temperature and stirred for 4 h, then concentrated and redissolved in EtOAc (10 mL). Saturated aqueous bicarbonate solution was added until neutral pH and the layers were separated. The aqueous phase was extracted with EtOAc (10 mL×2) and the combined organic extracts were washed with brine, dried (Na₂SO₄), filtered, and concentrated to give (1s,3s)-N,3-dimethyl-3-((7-(1-methyl-1H-pyrazol-4-yl)imidazo[1,2-c]pyrimidin-5-yl)oxy)cyclobutan-1-amine as an orange film (crude, assumed quantitative yield), which was carried forward without further purification. ESI-MS (M+H)+: 313.1.

### 3. Synthesis of N-methyl-N-((1s,3s)-3-methyl-3-((7-(1-methyl-1H-pyrazol-4-yl)imidazo[1,2-c]pyrimidin-5-yl)oxy)cyclobutyl)acrylamide

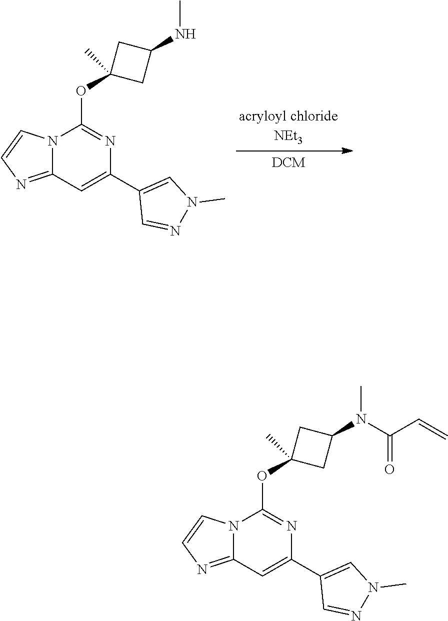 [SMILES: CN[C@H]1C[C@@](C)(Oc2nc(-c3cnn(C)c3)cc3nccn23)C1 ; C=CC(=O)N(C)[C@H]1C[C@@](C)(Oc2nc(-c3cnn(C)c3)cc3nccn23)C1]

To a solution of (1s,3s)-N,3-dimethyl-3-((7-(1-methyl-1H-pyrazol-4-yl)imidazo[1,2-c]pyrimidin-5-yl)oxy)cyclobutan-1-amine (213 mg, 682 μmol) in DCM (3.4 mL) was added triethylamine (285 μL, 2.05 mmol). The reaction mixture was cooled in an ice-water bath then acryloyl chloride (83 μL, 1.02 mmol) was added. The reaction mixture was stirred for 15 mins in the ice-water bath before warming to ambient temperature. The reaction mixture was loaded directly onto a silica gel column and purified (grading from 0-100% [3:1 EtOAc/EtOH] in heptanes) to give crude product. The recovered material was further purified by reverse phase HPLC (CHIRALPAK AD-H 30×250 mm, 5 um, gradient=30% MeOH CO₂ (flow rate: 100 mL/min, ABPR 120 bar, MBPR 40 psi, column temp 40° C.) to give N-methyl-N-((1s,3s)-3-methyl-3-((7-(1-methyl-1H-pyrazol-4-yl)imidazo[1,2-c]pyrimidin-5-yl)oxy)cyclobutyl)acrylamide as a film (6.3 mg, 3% yield over 2 steps). ESI-MS (M+H)⁺: 367.1. ¹H NMR (500 MHz, CHLOROFORM-d) δ: 7.94 (s, 1H), 7.83 (s, 1H), 7.59-7.49 (m, 2H), 7.44-7.34 (m, 1H), 6.56 (br dd, J=16.8 Hz, 10.7 Hz, 1H), 6.34 (br d, J=17.1 Hz, 1H), 5.73 (dd, J=10.4 Hz, 1.8 Hz, 1H), 4.81 (br s, 1H), 4.02-3.97 (m, 3H), 3.03 (br s, 3H), 2.93-2.81 (m, 2H), 2.70 (br s, 2H), 1.90 (s, 3H).

Basic PSR Method: To the reaction mixture was added 3 mL satd. NaHCO₃ and extracted by 3×3 mL EtOAc. The organic layers were combined and concentrated to dryness, then diluted with 2 mL DMSO, and passed through a 0.2 um syringe filter. The product was isolated via prep-HPLC using a Waters Sunfire Prep C18, 5 μm, 19 mm×100 mm column with mobile phase H₂O (A) and MeCN (B) and a gradient of 5-60% B (0.2% NH₄HCO₃ final v/v % modifier) with flow rate at 30 mL/min.

Acidic PSR Method: To the reaction mixture was added 3 mL satd. NaHCO₃ and extracted by 3×3 mL EtOAc. The organic layers were combined and concentrated to dryness, then diluted with 2 mL DMSO, and passed through a 0.2 um syringe filter. The product was isolated via prep-HPLC using a Waters Sunfire Prep C18, 5 μm, 19 mm×100 mm column with mobile phase H₂O (A) and MeCN (B) and a gradient of 5-60% B (0.2% TFA final v/v % modifier) with flow rate at 30 mL/min.

### D. Experimental Testing

### In Vitro BTK Kinase Assay: Btk-PolyGAT-LS Assay

The purpose of the BTK in vitro assay is to determine compound potency against BTK through the measurement of IC50. Compound inhibition is measured after monitoring the amount of phosphorylation of a fluorescein-labeled polyGAT peptide (Invitrogen PV3611) in the presence of active BTK enzyme (Upstate 14-552), ATP, and inhibitor. The BTK kinase reaction was done in a black 96 well plate (costar 3694). For a typical assay, a 24 μL aliquot of a ATP/peptide master mix (final concentration; ATP 10 μM, polyGAT 100 nM) in kinase buffer (10 mM Tris-HCl pH 7.5, 10 mM MgCl2, 200 μM Na3PO4, 5 mM DTT, 0.01% Triton X-100, and 0.2 mg/ml casein) is added to each well. Next, I pL of a 4-fold, 40X compound titration in 100% DMSO solvent is added, followed by adding 15 uL of BTK enzyme mix in 1X kinase buffer (with a final concentration of 0.25 nM). The assay is incubated for 30 minutes before being stopped with 28 μL of a 50 mM EDTA solution. Aliquots (5 uL) of the kinase reaction are transferred to a low volume white 384 well plate (Coming 3674), and 5 μL of a 2X detection buffer (Invitrogen PV3574, with 4 nM Tb-PY20 antibody, Invitrogen PV3552) is added. The plate is covered and incubated for 45 minutes at room temperature. Time resolved fluorescence (TRF) on Molecular Devices M5 (332 nm excitation; 488 nm emission; 518 nm fluorescein emission) is measured. IC50 values are calculated using a four parameter fit with 100% enzyme activity determined from the DMSO control and 0% activity from the EDTA control.

Table 1 shows the activity of the selected exemplary compounds of this invention in the in vitro Btk kinase assay, wherein each compound number corresponds to the compound numbering set forth in the Examples 1-236 herein.

“†” represents an IC₅₀ of greater than 1 μM and equal to or less than 10 μM. “††” represents an IC₅₀ of greater than 10 nM and equal to or less than 1 μM (10 nM<IC₅₀≤1 μM). “†††” represents an IC₅₀ of greater than 1 nM and equal to or less than 10 nM (1 nM<IC₅₀≤10 nM). “††††” represents an IC50 of less than 1 nM.

TABLE 1

| Example No. | IC50 (nM) |
|---|---|
| 195 | † |
| 2, 6, 9, 10, 17, 20, 23, 24, 26 E1, 27, 32, 38, 39, 40, 41, 42, 44, 45, 47, 49, 51, | †† |
| 55, 58, 69 E2, 71 E2, 74, 75, 87, 89, 90, 91, 94, 97, 104, 105, 106, 112, 114, 119, | |
| 122, 129, 131, 132, 135, 137, 144, 153, 154, 161, 168, 170, 171, 176, 178, 180, | |
| 182, 184, 191, 192, 194, 196, 198, 202, 203, 204, 208, 210, 216, 219, 222, 223 | |
| 1, 4, 11, 12, 14, 15, 18, 21, 22, 25, 33, 34, 35, 36, 37, 43, 46, 48, 52, 53, 54, 56, | ††† |
| 57, 62, 64, 65, 69 E1, 70, 71 E1, 73, 76, 78, 81, 82, 83, 86, 88, 93, 102, 103, 107, | |
| 109, 113, 115, 117, 121, 123, 124, 125, 130, 134, 136, 138, 141, 142, 143, 147, | |
| 148, 149, 152, 157, 158, 159, 164, 165, 169, 173, 183, 188, 189, 193, 199, 201, | |
| 205, 207, 212, 213, 215, 217, 218, 220, 230, 236 | |
| 3, 5, 7, 8, 13, 16, 19, 26 E2, 28, 29, 30, 31, 50, 59, 60, 61, 63, 66, 67, 68, 72 E1, | †††† |
| 77, 79, 80, 84, 85, 92, 95, 96, 98, 99. 100, 101, 108, 110, 111, 116, 118, 120, | |
| 126, 127, 128, 133, 139, 140, 145, 146, 150, 155, 156, 160, 162, 163, 166, 167, | |
| 172, 174, 175, 177, 179, 181, 185, 186, 187, 190, 195, 197, 200, 209, 211, 214, | |
| 221, 224, 225, 226, 227, 228, 229, 231, 232, 233, 234, 235 | |

In Vitro whole blood CD69 Assay

Human heparinized venous blood from health donors was aliquoted into 96-well plate and “spiked” with serial dilutions of formula I compounds in DMSO or with DMSO without drug. The final concentration of DMSO in all wells was 0.1%. The plate was incubated at 37° C. for 30 min. Drug-containing samples were stimulated with 0.1 μg/mL mouse anti-human IgD-dextran (1A62) or 20 μg/mL polyclonal rabbit F(ab′)2 anti-human IgD. Phosphate-buffered saline (PBS) was added to the negative control unstimulated sample and the plates were incubated overnight (18 to 22 hours) at 37° C. Cells were stained with fluorochrome-conjugated anti-CD19 and anti-CD69 antibodies. Lyse/fix solution was used to remove red blood cells by hypotonic lysis and to fix the remaining cells, which were then analyzed by flow cytometry. CD19+ B cells were gated and analyzed for CD69 expression. The percentage of B cells expressing CD69 was plotted versus the log 10 of the concentration of the drug and the best-fit curves (variable Hill slope) were generated to obtain the IC50 value.

Table 2 shows the activity of the selected exemplary compounds of this invention in the in vitro whole blood CD69 assay, wherein each compound number corresponds to the compound numbering set forth in the Examples 1-80 herein.

“†” represents an IC₅₀ of greater than 10 μM. “††” represents an IC₅₀ of greater than 1 μM and equal to or less than 10 μM (1 μM<IC₅₀≤10 μM). “†††” represents an IC₅₀ of less than 1 μM.

TABLE 2

| Example No. | IC50 (nM) |
|---|---|
| 1, 2, 3, 4, 5, 6, 9, 10, 11, 12, 14, 20, 23, | † |
| 24, 25, 26 E1, 54, 55, 56, 58, 60, 63 | |
| 7, 8, 15, 21, 22, 27, 31, 33, 39, 57, 62, | †† |
| 70, 71 E2, 72 E1, 81, 92, 98, 101, 103, | |
| 104, 113, 115, 123, 124, 125, 127, 129, | |
| 130, 141, 160, 164, 168, 169, 170, 171, | |
| 172, 173, 176, 178, 183, 185, 189, 191, | |
| 192, 193, 199, 207, 212, 213, 215, 217, | |
| 220, 221, 224, 234 | |
| 13, 16, 19, 26 E2, 28, 29, 30, 34, 43, 59, | ††† |
| 61, 64, 66, 67, 71 E1, 79, 85, 86, 93, 95, | |
| 96, 99, 100, 107, 108, 109, 110, 111, | |
| 116, 117, 118, 120, 121, 126, 128, 139, | |
| 140, 143, 145, 146, 148, 150, 158, 166, | |
| 167, 174, 175, 177, 179, 186, 187, 188, | |
| 190, 197, 200, 211, 214, 225, 226, 228, | |
| 229, 231, 232, 233, 235, 236 | |

The following compounds have yet to be submitted for analysis in the CD69 assay: 17, 18, 32, 35, 36, 37, 38, 40, 41, 42, 44, 45, 46, 47, 48, 49, 50, 51, 52, 53, 65, 68, 69 E1, 69 E2, 73, 74, 75, 76, 77, 78, 80, 82, 83, 84, 87, 88, 89, 90, 91, 94, 97, 102, 105, 106, 119, 112, 114, 119, 122, 131, 132, 133, 134, 135, 136, 137, 138, 142, 144, 147, 149, 151, 152, 153, 154, 155, 156, 157, 159, 161, 162, 163, 165, 180, 181, 182, 184, 194, 195, 196, 198, 201, 202, 203, 204, 205, 206, 208, 209, 210, 216, 218, 219, 222, 223, 227.

What is claimed is:

1. A compound represented by Formula (I′): 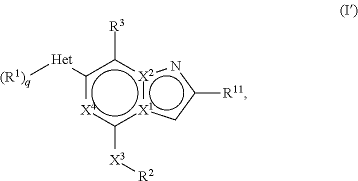
or a pharmaceutically acceptable salt thereof, wherein:
Het is phenyl, a 5-6 membered heteroaryl or a N—(C₁-C₃ alkyl)pyridonyl;
X⁰ is N, X¹ is C, X² is N and X⁴ is N; X⁰ is CR⁰, X¹ is C, X² is N and X⁴ is N; X⁰ is CR⁰, X¹ is N, X² is C and X⁴ is N; X⁰ is CR⁰, X¹ is N, X² is C and X⁴ is CH; or X⁰ is CR⁰, X¹ is C, X² is N and X⁴ is CH;
R⁰ is H, halo, methyl, halomethyl, cyclopropyl, CN or phenyl;
R¹ is H or C₁-C₃ alkyl, C₁-C₃-alkoxy, C₁-C₃ haloalkyl or a 4-7 membered monocyclic oxygen containing heterocycle;
R³ is H or halo;
X³ is absent, CH₂, CH₂CH₂, O, O—CH₂*, O—CH₂CH₂*, NH, N(CH₃)-*, CH₂N(CH₃)-* or NH—CH₂*, wherein “*” indicates the point of attachment to R²;
when X³ is absent, CH₂ or CH₂CH₂, R² is a 4-12 membered mono or bicyclic nitrogen-containing heterocycle bonded to the bicyclic core or X³ through a ring nitrogen atom (“N-attached”); when X³ is CH₂, CH₂CH₂, O, O—CH₂*, NH, N(CH₃)-*, CH₂N(CH₃)-* or NH—CH₂*, R² is a 4-12 membered mono or bicyclic nitrogen containing heterocycle bonded to X³ through a ring carbon atom (“C-attached”), a 4-7 membered mono or bicyclic oxygen containing heterocycle, a 3-12 membered mono or bicyclic carbocyclyl, or a 5-6 membered heteroaryl; and when X³ is O—CH₂—CH₂*, R² is absent, a 4-12 membered mono or bicyclic nitrogen containing heterocycle bonded to X³ through a ring carbon atom (“C-attached”) or a C₁-C₃ alkyl group, provided when R² is absent, X³ is directly connected to R⁴;
the N-attached 4-12 membered mono or bicyclic nitrogen-containing heterocycle, the 4-7 membered oxygen containing heterocycle, the 3-12 membered mono or bicyclic carbocycle, the 5-6 membered heteroaryl, and the C₁-C₃ alkyl group represented by R² are substituted with a group represented by R⁴ and optionally further substituted with one to three groups represented by R¹⁰, provided when the N-attached 4-12 membered mono or bicyclic nitrogen-containing heterocycle contains two ring nitrogen atoms, the N-attached 4-12 membered mono or bicyclic nitrogen-containing heterocycle represented by R² is optionally N-substituted with a group represented by R⁵ and optionally further substituted with one or two groups represented by R¹⁰;
the C-attached 4-12 membered mono or bicyclic nitrogen-containing heterocycle is N-substituted with a group represented by R⁵ and optionally further substituted with one to three groups represented by R¹⁰;
R⁴ is 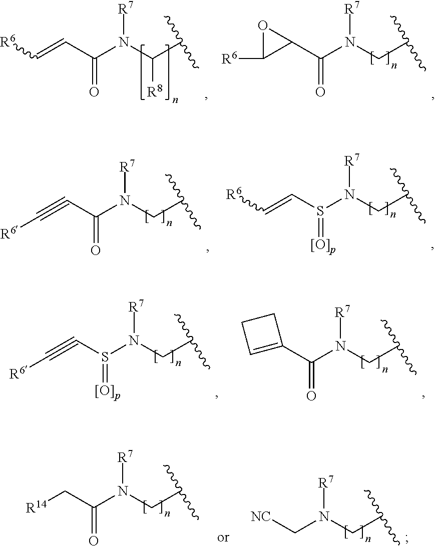
R⁵ is 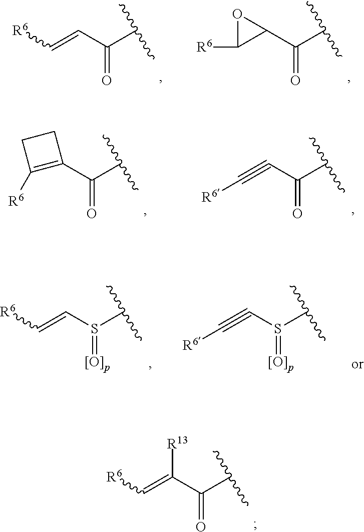
each R⁶ is independently H, CN, C₁-C₃ alkyl, C₁-C₃ haloalkyl, N(R^{a})₂ or CH₂N(R^{a})₂, wherein each R^{a} is independently H, C₁-C₃ alkyl or C₃-C₆ cycloalkyl;
each R⁶′ is independently H, C₁-C₃ alkyl, C₁-C₃ haloalkyl or C₃-C₆ cycloalkyl;
each R⁷ is independently H, C₁-C₂ alkyl, C₁-C₂ fluoroalkyl or C₃-C₆ cycloalkyl;
R⁸ is H or C₁-C₃ alkyl;
each R¹⁰ is halo, C₁-C₃ alkyl or C₃-C₆ cycloalkyl;
R¹¹ is H or N(R¹²)₂;
each R¹² is independently H or C₁-C₃ alkyl;
R¹³ is CN or F;
R¹⁴ is halo;
each n is independently 0 or 1;
each p is independently 1 or 2; and
q is 1 or 2.

2. The compound of claim 1, wherein the compound is represented by Formula (I): 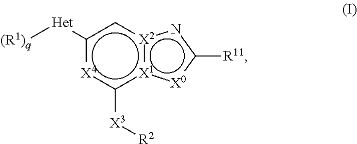
or a pharmaceutically acceptable salt thereof, wherein:
R⁰ is H, halo, methyl, halomethyl, cyclpropyl or CN;
X³ is absent, CH₂, CH₂CH₂, O, O—CH₂*, NH or NH—CH₂*, wherein “*” indicates the point of attachment to R²;
when X³ is absent, CH₂ or CH₂CH₂, R² is a 4-12 membered mono or bicyclic nitrogen-containing heterocycle bonded to the bicyclic core or X³ through a ring nitrogen atom (“N-attached”); and when X³ is CH₂, CH₂CH₂, O, O—CH₂*, NH or NH—CH₂*, R² is a 4-12 membered mono or bicyclic nitrogen containing heterocycle bonded to X³ through a ring carbon atom (“C-attached”), a 4-7 membered monocyclic oxygen containing heterocycle or a 3-12 membered mono or bicyclic carbocyclyl;
the N-attached 4-12 membered mono or bicyclic nitrogen-containing heterocycle, the 4-7 membered oxygen containing heterocycle and the 3-12 membered mono or bicyclic carbocycle represented by R² are substituted with a group represented by R⁴ and optionally further substituted with one or two groups represented by R¹⁰;
the C-attached 4-12 membered mono or bicyclic nitrogen-containing heterocycle is N-substituted with a group represented by R⁵ and optionally further substituted with one or two groups represented by R¹⁰;
R⁴ is 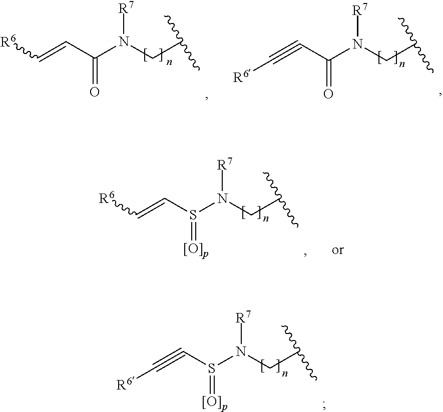
R⁵ is 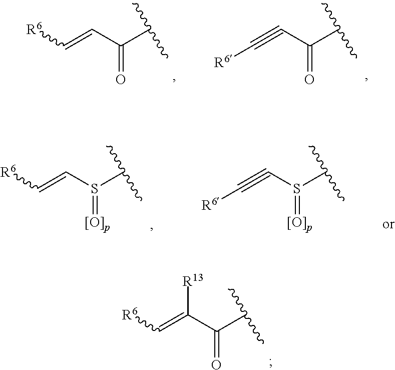
each R⁶ is independently H, C₁-C₃ alkyl, C₁-C₃ haloalkyl, N(R^{a})₂ or CH₂N(R^{a})₂, wherein each R^{a} is independently H or methyl;
each R⁶′ is independently H, C₁-C₃ alkyl or C₁-C₃ haloalkyl;
each R⁷ is independently H, C₁-C₂ alkyl or C₁-C₂ fluoroalkyl; and
each R¹⁰ is F or methyl.

3. The compound of claim 1 or 2, or a pharmaceutically acceptable salt thereof, wherein the R¹¹ is H or NH₂.

4. The compound of any one of claims 1-3, wherein the compound is represented by Formula (II): 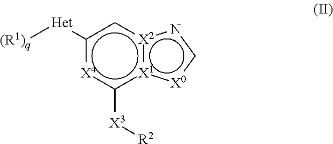
or a pharmaceutically acceptable salt thereof.

5. The compound of any one of claims 1-4 or a pharmaceutically acceptable salt thereof, wherein (R¹)_{q}-Het- is selected from:

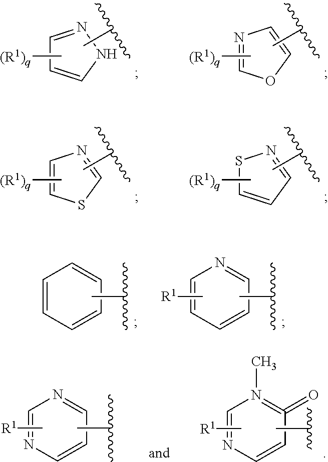

6. The compound of claim 1 or 2, wherein the compound is represented by Formula (III): 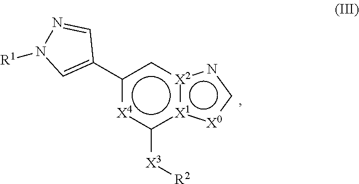
or a pharmaceutically acceptable salt thereof.

7. The compound of any one of claims 1-6 or a pharmaceutically acceptable salt thereof, wherein:
X⁰ is N, X¹ is C, X² is N and X⁴ is N; X⁰ is CH, X¹ is C, X² is N and X⁴ is N; X⁰ is CH, X¹ is N, X² is C and X⁴ is N; X⁰ is CR⁰, X¹ is N, X² is C and X⁴ is CH; or X⁰ is CH, X¹ is C, X² is N and X⁴ is CH;
X³ is absent, O, O—CH₂*, NH or NH—CH₂*, wherein “*” indicates the point of attachment to R²;
when X³ is absent, R² is a 4-12 membered monocyclic or bicyclic nitrogen-containing heterocycle bonded to the bicyclic core or X³ through a ring nitrogen atom (“N-attached”); and when X³ is O, O—CH₂* or NH—CH₂*, R² is a 4-12 membered monocyclic or bicyclic nitrogen containing heterocycle bonded to X³ through a ring carbon atom (“C-attached”), a 4-7 membered monocyclic oxygen containing heterocycle or a 3-12 membered monocyclic or bicyclic carbocyclyl;
the N-attached 4-12 membered monocyclic or bicyclic nitrogen-containing heterocycle, the 4-7 membered monocyclic oxygen-containing and the 3-12 membered monocyclic or bicyclic carbocycle represented by R² are substituted with a group represented by R⁴ and optionally further substituted with one or two groups represented by R¹⁰;
the C-attached 4-12 membered monocyclic or bicyclic nitrogen-containing heterocycle is N-substituted with a group represented by R⁵ and optionally further substituted with one or two groups represented by R¹⁰.

8. The compound of any one of claims 1-7, wherein the compound is represented by Formula (IV): 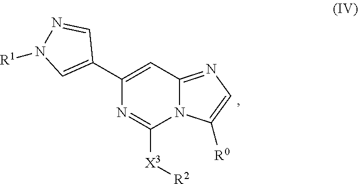
or a pharmaceutically acceptable salt thereof.

9. The compound of any of claims 1-7, wherein the compound is represented by Formula (V): 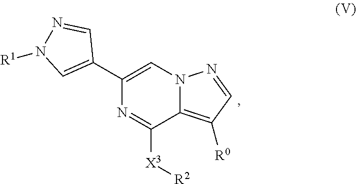
or a pharmaceutically acceptable salt thereof.

10. The compound of any one of claims 1-7, wherein the compound is represented by Formula (VI): 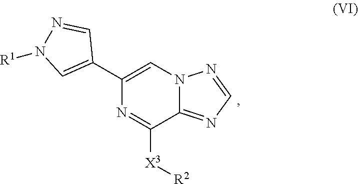
or a pharmaceutically acceptable salt thereof.

11. The compound of any one of claims 1-7, wherein the compound is represented by Formula (VII): 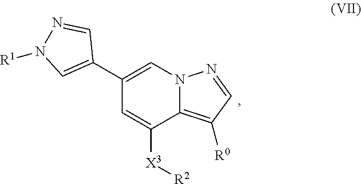
or a pharmaceutically acceptable salt thereof.

12. The compound of any one of claims 1-7, wherein the compound is represented by Formula (VIII): 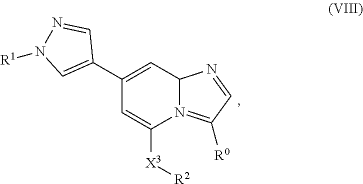
or a pharmaceutically acceptable salt thereof.

13. The compound or pharmaceutically acceptable salt thereof of any one of claims 1-12 wherein X³ is a bond and R² is a 4-12 membered monocyclic or bicyclic nitrogen-containing heterocycle bonded to the bicyclic core through its ring nitrogen atom and the 4-12 membered monocyclic or bicyclic nitrogen-containing heterocycle represented by R² is substituted with a group represented by R⁴ and optionally further substituted with one or two groups represented by R¹⁰.

14. The compound or pharmaceutically acceptable salt thereof of claim 13, wherein X³ is a bond and R² is a 7-10 membered bicyclic nitrogen-containing heterocycle bonded to the bicyclic core through its ring nitrogen atom and the 7-10 membered bicyclic nitrogen-containing heterocycle represented by R² is substituted with a group represented by R⁴ and optionally further substituted with one or two groups represented by R¹⁰.

15. The compound or pharmaceutically acceptable salt thereof of claim 14, wherein the 7-10 membered bicyclic nitrogen-containing heterocycle represented by R² is azaspiro[2.4]heptanylene substituted with a group represented by R⁴ and optionally further substituted with a group represented by R¹⁰.

16. The compound or pharmaceutically acceptable salt thereof of any one of claims 1-12, wherein X³ is a bond and R² is a 4-7 membered monocyclic nitrogen-containing heterocycle bonded to the bicyclic core through its ring nitrogen atom and the 4-7 membered monocyclic nitrogen-containing heterocycle represented by R² is substituted with a group represented by R⁴ and optionally further substituted with a group represented by R¹⁰.

17. The compound or pharmaceutically acceptable salt thereof of claim 16, wherein the 4-7 membered monocyclic nitrogen-containing heterocycle represented by R² is azetidinylene, pyrrolindinylene, piperidinylene, azapanylene or oxazapanylene, each substituted with a group represented by R⁴ and optionally further substituted with one or two groups represented by R¹⁰.

18. The compound of any one of claim 17, wherein the compound is represented by a structural formula selected from: 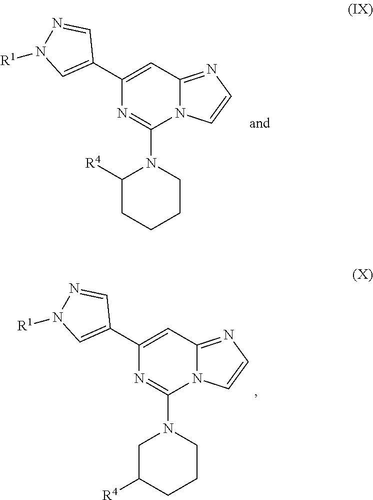
or pharmaceutically acceptable salt thereof.

19. The compound of claim 17, wherein the compound is represented by a structural formula selected from: 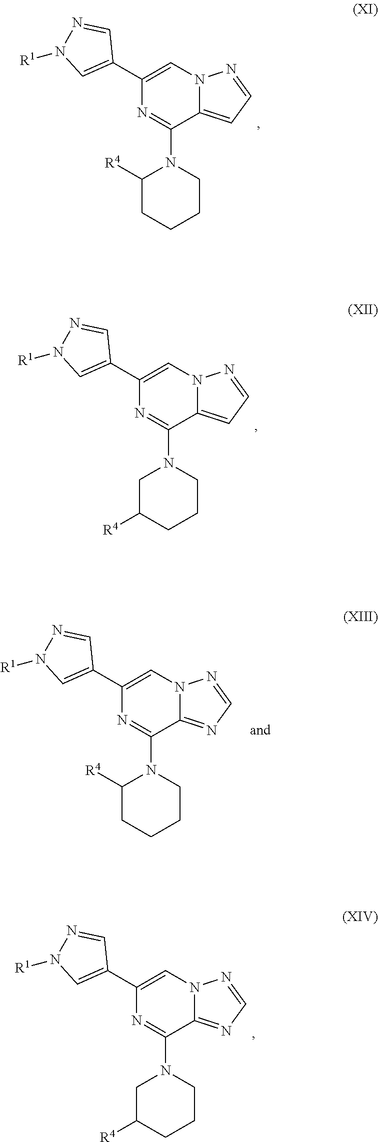
or pharmaceutically acceptable salt thereof.

20. The compound or pharmaceutically acceptable salt thereof of any one of claims 1-19, wherein R⁶ is H, CH₃ or CH₂Cl and p is 2.

21. The compound or pharmaceutically acceptable salt thereof of any one of claims 1-20, wherein R⁴ is CH₂NHC(O)C≡CH, CH₂NHC(O)CH═CH₂, N(CH₃)C(O)C≡CH, NHC(O)CH═CH₂, NHC(O)C≡CH or NHC(O)CH═CHCH₂Cl.

22. The compound or pharmaceutically acceptable salt thereof of any one of claims 1-20, wherein R⁴ is CH₂NHC(O)C≡CH, CH₂NHC(O)CH═CH₂, N(CH₃)C(O)C≡CH or CH₂N(R⁷)C(O)CH═CHCH₂Cl.

23. The compound or pharmaceutically acceptable salt thereof of any one of claims 1-12, wherein X³ is O, O—CH₂*, O—CH₂CH₂*, NH, NH—CH₂*, N(CH₃), or CH₂N(CH₃)-*, R² is a 4-12 membered mono or bicyclic nitrogen containing heterocycle bonded to X³ through a ring carbon atom (“C-attached”) and the C-attached 4-12 membered nitrogen-containing heterocycle is N-substituted with a group represented by R⁵ and optionally further substituted with one to three groups represented by R¹⁰.

24. The compound or pharmaceutically acceptable salt thereof of any one of claims 1-12, wherein X³ is O, O—CH₂*, NH or NH—CH₂*, R² is a 4-12 membered nitrogen containing heterocycle bonded to X³ through a ring carbon atom (“C-attached”) and the C-attached 4-12 membered nitrogen-containing heterocycle is N-substituted with a group represented by R⁵ and optionally further substituted with one or two groups represented by R¹⁰.

25. The compound or pharmaceutically acceptable salt thereof of any one of claims 1-12, 23 and 24, wherein X³ is O or O—CH₂*.

26. The compound or pharmaceutically acceptable salt thereof of any one of claims 1-12 or 23-25, wherein the C-attached 4-12 membered nitrogen containing heterocycle represented by R² is a 4-7 membered monocyclic optionally containing one ring oxygen or one ring sulfur atom, a 6-10 membered fused bicyclic, an 8-12 membered spirocycle or 7-10 bridged bicyclic, and the C-attached 4-12 membered nitrogen containing heterocycle represented by R² is N-substituted with a group represented by R⁵ and is optionally further substituted with one or two groups represented by R¹⁰.

27. The compound or pharmaceutically acceptable salt thereof of any one of claims 1-12 or 23-26, wherein the C-attached 4-12 membered nitrogen containing heterocycle represented by R² is azaspiro[3.3]heptanylene, azaspiro[3.5]nonanylene, azaspiro[4.4]nonanylene, azaspiro[3.4]octanylene, azetidinylene, pyrrolindinylene, piperidinylene, azapanylene, diazepanylene, morpholinylene, octahydrocyclopenta[c]pyrrolylene, oxazapanylene, azabicyclo[3.2.0]heptanylene, azabicyclo[2.2.1]heptanylene, azabicyclo[3.1.1]heptanylene, azabicyclo[3.2.1]octanylene, azabicyclo[4.2.0]octanylene, azatricyclo[4.1.1.03,7]octylene, azabicyclo[3.2.0]heptanylene, azabicyclo[2.1.1]heptanylene, azabicyclo[2.1.1]hexanylene, azabicyclo[3.1.0]hexanylene, 2λ2-azaspiro[3.4]octylene or octahydrocyclopenta[c]pyrollene and the C-attached 4-12 membered nitrogen containing heterocycle represented by R² is N-substituted with a group represented by R⁵ and is optionally further substituted with one or two groups represented by R¹⁰.

28. The compound or pharmaceutically acceptable salt thereof of any one of claims 1-12 or 23-26, wherein the C-attached 4-12 membered nitrogen containing heterocycle represented by R² is azetidinylene, pyrrolindinylene, piperidinylene, azapanylene, oxazapanylene, azabicyclo[3.2.1]octanylene, azatricyclo[4.1.1.03,7]octylene, azabicyclo[3.2.0]heptanylene, azabicyclo[3.1.0]hexanylene, 2λ2-azaspiro[3.4]octylene or octahydrocyclopenta[c]pyrollene and the C-attached 4-12 membered nitrogen containing heterocycle represented by R² is N-substituted with a group represented by R⁵ and is optionally further substituted with one or two groups represented by R¹⁰.

29. The compound or pharmaceutically acceptable salt thereof of claim 27, wherein the C-attached 4-12 membered mono or bicyclic nitrogen containing heterocycle represented by R² is selected from: 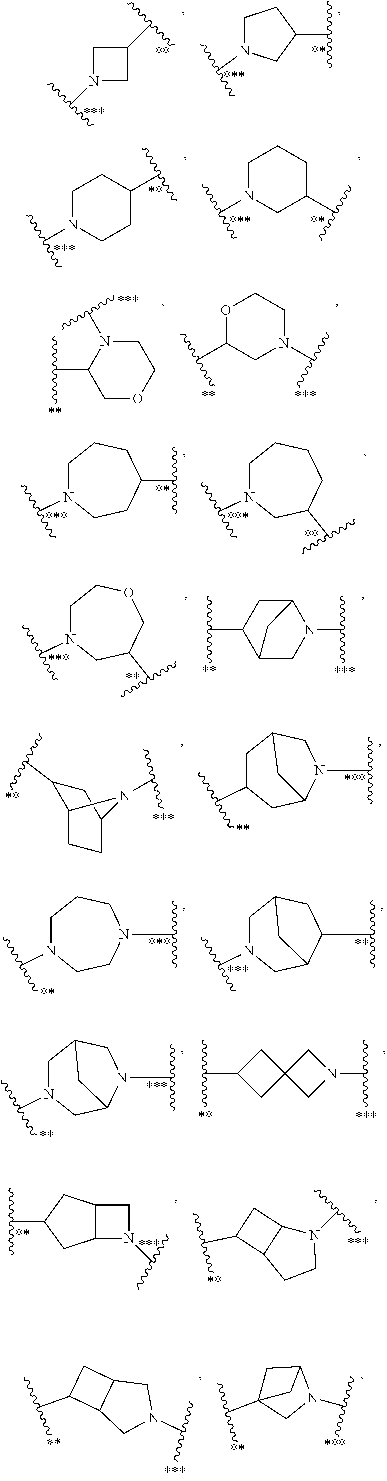 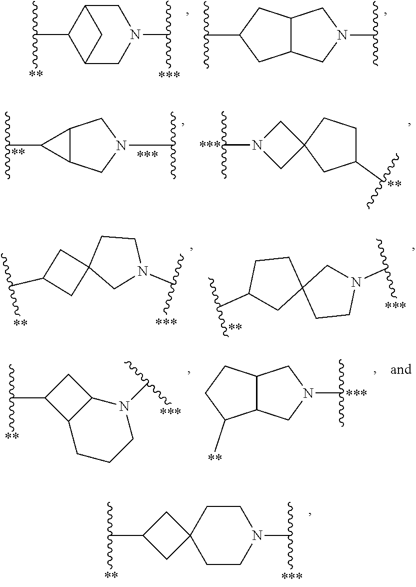
wherein “**” indicates the point of attachment to X³; and “***” indicates the point of attachment to R⁵, wherein each group represented by R² is optionally further substituted with one to three groups represented by R¹⁰.

30. The compound or pharmaceutically acceptable salt thereof of claim 28, wherein the C-attached 4-12 membered mono or bicyclic nitrogen containing heterocycle represented by R² is selected from: 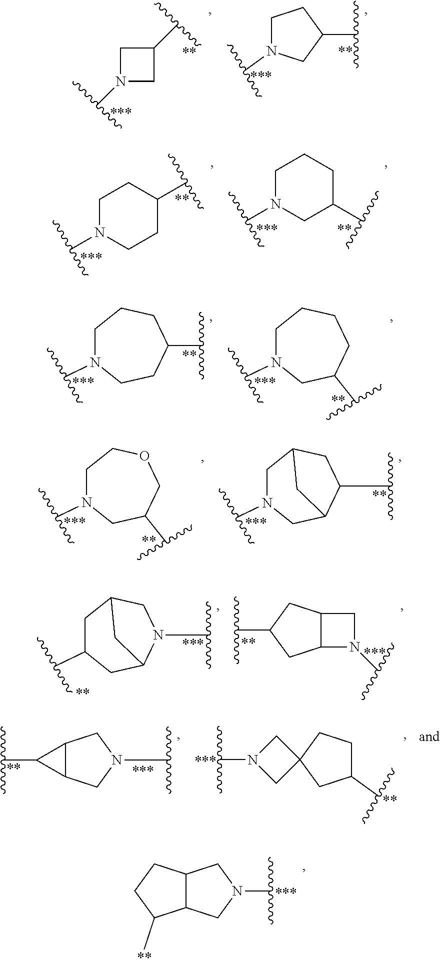
wherein “**” indicates the point of attachment to X³; and “***” indicates the point of attachment to R⁵, wherein each group represented by R² is optionally further substituted with one or two groups represented by R¹⁰.

31. The compound or pharmaceutically acceptable salt thereof of any one of claims 1-12 and 23-30, wherein the stereochemical configuration of the ring carbon atom in the C-attached 4-12 membered nitrogen containing heterocycle represented by R² that is bonded to X³ is R.

32. The compound or pharmaceutically acceptable salt thereof of any one of claims 1-12 or 23-30, wherein the stereochemical configuration at the ring carbon atom in the C-attached 4-12 membered nitrogen containing heterocycle represented by R² that is bonded to X³ is S.

33. The compound or pharmaceutically acceptable salt thereof of any one of claims 1-12 or 23-32, wherein R⁶ and R⁶′ are independently H, CH₃ or CH₂Cl and p is 2.

34. The compound or pharmaceutically acceptable salt thereof of any one of claims 1-12 or 23-33, wherein R⁵ is SO₂CH═CH₂, SO₂CH═CHCH₃, SO₂CH≡CHCH₂Cl, SO₂C≡CH, SO₂C≡CCH₃, SO₂C≡CCH₂Cl, COCH═CH₂, COCH═CHCH₃, COCH═CHCH₂Cl, CO—C≡CH, CO—C≡CCH₃, CO—C≡CCH₂Cl, COCF═CH₂, COCF═CHCH₃, COCF═CHCH₂Cl,

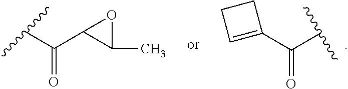

35. The compound or pharmaceutically acceptable salt thereof of any one of claims 1-12 or 23-33, wherein R⁵ is SO₂CH═CH₂, SO₂CH═CHCH₃, SO₂CH═CHCH₂Cl, SO₂C≡CH, SO₂C≡CCH₃, SO₂C≡CCH₂Cl, COCH═CH₂, COCH═CHCH₃, COCH═CHCH₂Cl, CO—C≡CH, CO—C≡CCH₃, CO—C≡CCH₂Cl, COCF═CH₂, COCF═CHCH₃ or COCF═CHCH₂Cl.

36. The compound or pharmaceutically acceptable salt thereof of claim 35, wherein R⁵ is SO₂CH═CH₂, SO₂CH═CHCH₃, COCH═CH₂, COCF═CH₂, COCH═CHCH₂Cl, CO—C≡CH or CO—C≡CCH₃.

37. The compound or pharmaceutically acceptable salt thereof of any one of claims 1-12, wherein X³ is O, O—CH₂*, NH or NH—CH₂*, R² is a 3-12 membered mono or bicyclic carbocyclyl, a 4-7 membered mono or bicyclic oxygen containing heterocycle or a 5-6 membered heteroaryl and the 3-12 membered mono or bicyclic carbocycle, the 4-7 membered mono or bicyclic oxygen containing heterocycle and the 5-6 membered heteroaryl represented by R² is substituted with a group represented by R⁴ and optionally further substituted with one to three groups represented by R¹⁰.

38. The compound or pharmaceutically acceptable salt thereof of any one of claims 1-12, wherein X³ is O, O—CH₂*, NH or NH—CH₂*, R² a 4-7 membered mono or bicyclic oxygen containing heterocycle or a 5-6 membered heteroaryl and the 4-7 membered mono or bicyclic oxygen containing heterocycle and the 5-6 membered heteroaryl represented by R² are substituted with a group represented by R⁴ and optionally further substituted with one to three groups represented by R¹⁰.

39. The compound or pharmaceutically acceptable salt thereof of claim 38, wherein the 4-7 membered mono or bicyclic oxygen containing heterocycle is oxabicyclo [3.1.1]heptanylene or tetrahydro-2H-pyranylene, each substituted with a group represented by R⁴ and optionally further substituted with one or two groups represented by R¹⁰; and the 5-6 membered heteroaryl is pyridinylene substituted with a group represented by R⁴ and optionally further substituted with one to three groups represented by R¹⁰.

40. The compound or pharmaceutically acceptable salt thereof of claim 38, wherein R² is selected from: 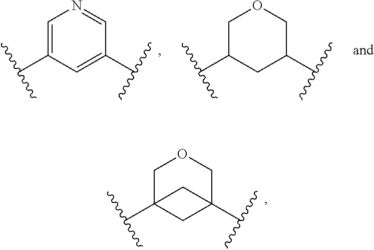 [SMILES: *c1cncc(*)c1 ; *C1COCC(*)C1 ; *C12COCC(*)(C1)C2]
each substituted with a group represented by R⁴ and optionally further substituted with one or two groups represented by R¹⁰.

41. The compound or pharmaceutically acceptable salt thereof of any one of claims 1-12, wherein X³ is O, O—CH₂*, NH or NH—CH₂*, R² is a 3-12 membered mono or bicyclic carbocyclyl and the 3-12 membered mono or bicyclic carbocycle represented by R² is substituted with a group represented by R⁴ and optionally further substituted with one or two groups represented by R¹⁰.

42. The compound or pharmaceutically acceptable salt thereof of any one of claims 1-12 and 37-41, wherein X³ is O or O—CH₂*.

43. The compound or pharmaceutically acceptable salt thereof of any one of claims 1-12 and 37-42, wherein R² is phenylene, C₃-C₇ cycloalkylene or C₆-C₉ bicyclic saturated carbocycle and the phenylene, C₃-C₇ cycloalkylene and C₆-C₉ bicyclic saturated carbocycle represented by R² is substituted with a group represented by R⁴ and optionally further substituted with one or two groups represented by R¹⁰.

44. The compound or pharmaceutically acceptable salt thereof of any one of claims 1-12 or 41-43, wherein R² is phenylene or C₄-C₇ cycloalkylene substituted with a group represented by R⁴ and optionally further substituted with one or two groups represented by R¹⁰.

45. The compound or pharmaceutically acceptable salt thereof of any one of claims 1-12 or 37-44, wherein X³ is O.

46. The compound or pharmaceutically acceptable salt thereof of any one of claims 1-12 or 41-45, wherein R² is phenylene, cyclobutylene, cyclohexylene, cyclopentylene, cyclopropylene, bicyclo[3.3.1]heptylene, bicyclo[2.2.1]heptanylene, bicyclo[4.1.0]heptanylene or bicyclo[2.1.1]hexanylene, each of which is substituted with a group represented by R⁴ and optionally further substituted with one or two groups represented by R¹⁰.

47. The compound or pharmaceutically acceptable salt thereof of any one of claims 1-12 or 41-45, wherein R² is phenylene, cyclobutylene, cyclohexylene or bicyclo[3.3.1]heptylene substituted with a group represented by R⁴ and optionally further substituted with one or two groups represented by R¹⁰.

48. The compound or pharmaceutically acceptable salt thereof of any one of claims 1-12 or 41-45, wherein R² is, 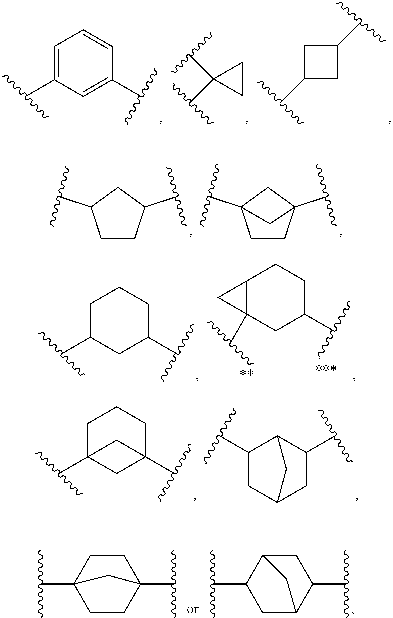
wherein “**” indicates the point of attachment to X³; and “***” indicates the point of attachment to R⁴, wherein the group represented by R² is optionally substituted with one or two groups represented by R¹⁰.

49. The compound or pharmaceutically acceptable salt thereof of any one of claims 1-12 or 41-45, wherein R² is 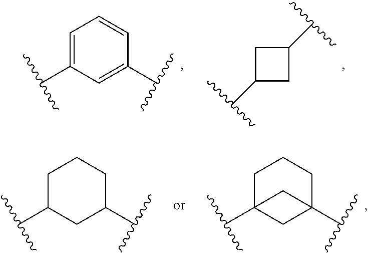 [SMILES: *c1cccc(*)c1 ; *C1CC(*)C1 ; *C1CCCC(*)C1 ; *C12CCCC(*)(C1)C2]
wherein the group represented by R² is optionally substituted with one or two groups represented by R¹⁰.

50. The compound or pharmaceutically acceptable salt thereof of any one of claims 1-12 and 41-49, wherein R⁶ and R⁶′ are independently H, CN, CH₃, CH₂Cl, CF₃, cyclopropyl or CH₂N(R^{a})₂.

51. The compound or pharmaceutically acceptable salt thereof of claim 50, wherein R^{a} are each independently selected from —CH₃ and cyclopropyl.

52. The compound or pharmaceutically acceptable salt thereof of any one of claims 1-12 or 41-49, wherein R⁶ and R⁶′ are independently H, CH₃ or CH₂Cl.

53. The compound or pharmaceutically acceptable salt thereof of any one of claims 1-12 and 41-51, wherein R⁴ is NHC(O)CH═CH₂, N(CH₃)C(O)CH═CH₂, NHC(O)CH═CHCH₃, N(CH₃)C(O)CH═CHCH₃, N(CH₃)C(O)CH═CHCN, NHC(O)C≡CH, N(CH₃)C(O)C≡CH, N(H)C(O)C≡CCH₃, N(CH₃)C(O)C≡CCH₃, N(CH₂CH₂F)C(O)CH═CH₂, N(CH₂CH₂F)C(O)CH═CHCH₃, N(CH₂CH₂F)C(O)C≡CH, N(CH₂CH₂F)C(O)C≡CCH₃, CH₂N(CH₃)C(O)CH═CH₂, N(CH₂CHF₂)C(O)CH═CH₂, N(CH₃)C(O)CH═CHCH₂Cl, NHC(O)CH═CHCF₃, N(CH₃)C(O)CH═CHCF₃, NHC(O)C≡C-cyclopropyl, NHC(O)CH═CHCH₂N(CH₃)-cyclobutyl, N(CH₂CHF₂)C(O)CH═CHCH₂N(CH₃)₂, N(cyclopropyl)C(O)CH═CH₂, N(CH₃)C(O)CH₂Cl, N(CH₃)CH₂CN, 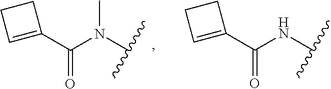
CH₂NHC(O)CH═CH₂, or CH(CH₃)NHC(O)CH═CH₂.

54. The compound of pharmaceutically acceptable salt thereof of any one of claims 1-12 or 41-52, wherein R⁴ is NHCOCH═CH₂, N(CH₃)COCH═CH₂, NHCOCH═CHCH₃, N(CH₃)COCH═CHCH₃, N(H)COC≡CH, N(CH₃)COC≡CH, N(H)COC≡CCH₃, N(CH₃)COC≡CCH₃, N(CH₂CH₂F)COCH═CH₂, N(CH₂CH₂F)COCH═CHCH₃, N(CH₂CH₂F)COC≡CH or N(CH₂CH₂F)COC≡CCH₃.

55. The compound or pharmaceutically acceptable salt thereof of any one of claims 1-12 or 41-52, wherein R⁴ is NHC(O)C≡CH, NHC(O)C≡CCH₃, NHC(O)CH═CH₂, N(CH₃)COCH═CH₂, N(CH₃)COC≡CCH₃ or N(CH₂CH₂F)COCH═CH₂.

56. The compound or pharmaceutically acceptable salt thereof of any one of claims 1-12 or 41-55, wherein the stereochemical configuration of the ring carbon atom in the C-attached 3-12 membered carbocycle represented by R² that is bonded to X³ is R.

57. The compound or pharmaceutically acceptable salt thereof of any one of claims 1-12 or 41-55, wherein the stereochemical configuration of the ring carbon atom in the C-attached 3-12 membered carbocycle represented by R² that is bonded to X³ is S.

58. The compound or pharmaceutically acceptable salt thereof of any one of claims 1-12 or 41-55, wherein X³ and R⁴ are orientated trans.

59. The compound or pharmaceutically acceptable salt thereof of any one of claims 1-12 or 41-55, wherein X³ and R⁴ are orientated cis.

60. The compound or pharmaceutically acceptable salt thereof of any one of claims 1-12, wherein X³ is O—CH₂CH₂*, and R² is C₁-C₃ alkyl group substituted with a group represented by R⁴ and optionally further substituted with one or two groups represented by R^{10′} or R² is absent and X³ is directly connected to R⁴.

61. The compound or pharmaceutically acceptable salt thereof of claim 60, R² is selected from **-CH₂-***, **-CH₂CH(CH₃)-***, wherein “**” represents a point of attachment to X³, and “***” represents a point of attachment to R⁴.

62. The compound or pharmaceutically acceptable salt thereof of claim 60 or 61, wherein R⁴ is N(CH₃)C(O)CH═CH₂.

63. The compound or pharmaceutically acceptable salt thereof of any one of claims 1-62, wherein R¹ is H or C₁-C₃ alkyl, C₁-C₃ fluoroalkyl or a 4-7 membered monocyclic oxygen containing heterocycle.

64. The compound or pharmaceutically acceptable salt thereof of any one of claims 1-63, wherein R¹ is H, CH₃, CH(CH₃)₂, CHF₂, CF₃, oxetanyl or tetrahydrofuranyl.

65. The compound or pharmaceutically acceptable salt thereof of any one of claims 1-63, wherein R¹ is H, CH₃, CH(CH₃)₂, CHF₂, oxetanyl or tetrahydrofuranyl.

66. The compound or pharmaceutically acceptable salt thereof of any one of claims 1-65, wherein R⁰ is H, F, CN, CH₃, CF₃, cyclopropyl or phenyl.

67. The compound or pharmaceutically acceptable salt thereof of any one of claims 1-65, wherein R⁰ is H, F, CN, CH₃ or CF₃.

68. The compound or pharmaceutically acceptable salt thereof of any one of claims 1-67, wherein R⁷ is selected from H, CH₃, CH₂CH₃, CH₂CHF₂ and cyclopropyl.

69. The compound or pharmaceutically acceptable salt thereof of any one of claims 1-68, wherein R⁸ is H or CH₃.

70. The compound or pharmaceutically acceptable salt thereof of any one of claims 1-69, wherein R¹⁰ is F, Cl, CH₃ or cyclopropyl.

71. The compound or pharmaceutically acceptable salt thereof of any one of claims 1-70, wherein R¹⁴ is Cl.

72. The compound of claim 1, wherein the compound is represented by Formula (XV): 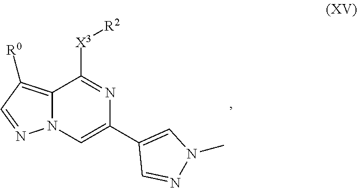
or a pharmaceutically acceptable salt thereof, wherein:
R⁰ is H, halo or cyclopropyl;
X³ is O or O—CH₂*;
R² is a 4-7 membered monocyclic or bicyclic saturated carbocyclyl and the 4-7 membered monocyclic or bicyclic saturated carbocyclyl represented by R² is substituted with a group represented by R⁴ and optionally further substituted with one or two R¹⁰, or
R² is a 7-9 membered bicyclic nitrogen containing heterocycle bonded to X³ through a ring carbon atom (“C-attached”) and the C-attached 7-9 membered bicyclic nitrogen containing heterocycle is substituted with a group represented by R⁵ and optionally further substituted with one or two R¹⁰;
R⁴ is N(R⁷)C(O)C≡CCH₃, N(R⁷)C(O)CH═CH₂,
R⁵ is C(O)CH═CH₂,
R⁷ is H, C₁-C₂alkyl, or C₁-C₂haloalkyl; and
R¹⁰ is C₁-C₃alkyl.

73. The compound or pharmaceutically acceptable salt thereof of claim 72, wherein X³ is O.

74. The compound or pharmaceutically acceptable salt thereof of claim 72 or 73, wherein R² is cyclobutylene, cyclohexylene, cyclopentylene or bicyclo [2.1.1]hexanylene, each of which is substituted with a group represented by R⁴ and optionally further substituted with one or two R¹⁰.

75. The compound or pharmaceutically acceptable salt thereof of claim 72 or 73, wherein R² 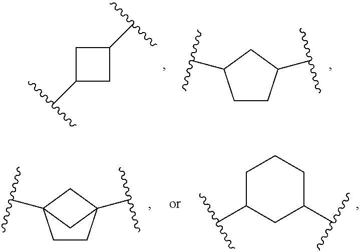 [SMILES: *C1CC(*)C1 ; *C1CCC(*)C1 ; *C12CCC(*)(C1)C2 ; *C1CCCC(*)C1]
wherein the group represented by R² is optionally further substituted with one or two groups represented by R¹⁰.

76. The compound or pharmaceutically acceptable salt thereof of claim 72 or 73, wherein R² is azabicyclo[3.2.1]octanylene, azabicyclo[3.1.1]heptanylene or azabicyclo[3.2.0]heptanylene, each of which is substituted with a group represented by R⁵ and optionally further substituted with one or two R¹⁰.

77. The compound or pharmaceutically acceptable salt thereof of claim 76, wherein R² is 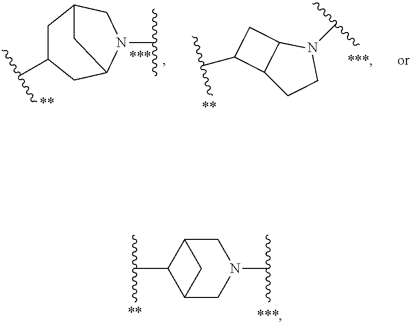 [SMILES: *C1CC2CC(C1)N(*)C2 ; *C1CC2C1CCN2* ; *C1C2CC1CN(*)C2]
wherein “**” indicates the point of attachment to X³; and “***” indicates the point of attachment to R⁵, wherein each group represented by R² is optionally further substituted with one or two groups represented by R¹⁰.

78. The compound or pharmaceutically acceptable salt thereof of any one of claims 72-77, wherein R⁷ is H, CH₃ or CH₂CHF₂.

79. The compound or pharmaceutically acceptable salt thereof of any one of claims 72-78, wherein R¹⁰ is CH₃.

80. A pharmaceutical composition comprising a compound of any one of claims 1-79 or a pharmaceutically acceptable salt thereof, and a pharmaceutically acceptable excipient.

81. A method of treating a disorder responsive to inhibition of Bruton's tyrosine kinase in a subject comprising administering to the subject an effective amount of the compound according to any of claims 1-79, or a pharmaceutically acceptable salt thereof, or a pharmaceutical composition according to claim 80.

82. The method of claim 81, wherein the disorder is an autoimmune disorder.

83. The method of claim 82, wherein the autoimmune disorder is rheumatoid arthritis.

84. The method of claim 82, wherein the autoimmune disorder is systemic lupus erythematosus.

85. The method of claim 81, wherein the disorder is atopic dermatitis.

86. The method of claim 81, wherein the disorder is leukemia or lymphoma.
